(19) United States
(12) Patent Application Publication
(10) Pub. No.: US 2024/0239774 A1
(43) Pub. Date: Jul. 18, 2024

(54) SOLUBLE ADENYLYL CYCLASE (SAC) INHIBITORS AND USES THEREOF

(71) Applicants: Cornell University, Ithaca, NY (US); Tri-Institutional Therapeutics Discovery Institue, Inc., New York, NY (US)

(72) Inventors: Melanie Balbach, New York, NY (US); Jochen Buck, Old Greenwich, CT (US); Lonny R. Levin, New York, NY (US); Jonathan Hale Zippin, Scarsdale, NY (US); Clemens Steegborn, Bayreuth (DE); Makoto Fushimi, Harrison, NY (US); David John Huggins, New York, NY (US); Nigel Liverton, Harleysville, PA (US); Peter T. Meinke, Scotch Plains, NJ (US); Mayako Michino, New York, NY (US); Michael Miller, Scotch Plains, NJ (US)

(73) Assignees: Cornell University, Ithaca, NY (US); Tri-Institutional Therapeutics Discovery Institue, Inc., New York, NY (US)

(21) Appl. No.: 18/288,368

(22) PCT Filed: Apr. 27, 2022
(86) PCT No.: PCT/US2022/026520

§ 371 Date: Oct. 25, 2023

Related U.S. Application Data
(60) Provisional application No. 63/180,876, filed on Apr. 28, 2021.

(51) Int. Cl. C07D 409/14; A61K 31/506; A61K 31/5377; A61K 31/5386; A61P 15/16; A61P 29/00; C07D 401/14; C07D 403/04; C07D 403/14; C07D 405/14; C07D 413/14; C07D 417/14; C07D 491/107; C07D 498/18

(52) U.S. Cl. CPC C07D 409/14; A61K 31/506; A61K 31/5377; A61K 31/5386; A61P 15/16; A61P 29/00; C07D 401/14; C07D 403/04; C07D 403/14; C07D 405/14; C07D 413/14; C07D 417/14; C07D 491/107; C07D 498/18

(57) ABSTRACT

Provided herein are soluble adenylyl cyclase (sAC) inhibitors and uses thereof. In one aspect, provided herein are compounds of Formula (I), and pharmaceutically acceptable salts, hydrates, solvates, polymorphs, co-crystals, tautomers, stereoisomers, isotopically labeled derivatives, and prodrugs thereof, and pharmaceutical compositions thereof. The compounds provided herein are soluble adenylyl cyclase (sAC) inhibitors and are therefore useful for the treatment and/or prevention of various diseases and conditions (e.g., ocular conditions (e.g., ocular hypotony), liver diseases (e.g., non-alcoholic steatohepatitis (NASH)), inflammatory diseases, autoimmune diseases (e.g., psoriasis)). Compounds provided herein are also useful as contraceptive agents (e.g., for male and female contraception).

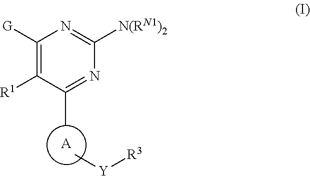

### RELATED APPLICATIONS

This application claims priority under 35 U.S.C. § 119(e) to United States Provisional Patent Application, U.S. Ser. No. 63/180,876, filed Apr. 28, 2021, the entire contents of which is incorporated herein by reference.

### GOVERNMENT SUPPORT

This invention was made with government support under HD088571, HD100549, and EY025810, awarded by the National Institutes of Health. The government has certain rights in the invention.

### BACKGROUND OF THE INVENTION

Cyclic AMP (cAMP) (known as “second messenger”) is implicated in a variety of physiological processes, including different aspects of cell proliferation and apoptosis, differentiation, migration, development, ion transport, pH regulation, and gene expression. Cyclic AMP is produced from ATP by adenylyl cyclases (ACs) and degraded by catabolizing phosphodiesterases (PDEs). Currently, there are two known, distinct types of AC in mammals: bicarbonate-regulated soluble adenylyl cyclase (sAC, ADCY10) and G protein regulated transmembrane adenylyl cyclases (tmACs; ADCY1-9). Soluble adenylyl cyclase (sAC) is a source of cAMP in intracellular microdomains and is found distributed through the cytoplasm and in cellular organelles, including inside the nucleus and the mitochondrial matrix. Inside the matrix, the sAC-defined intramitochondrial cAMP signaling cascade regulates ATP production, while in the cytoplasm, sAC has been identified as the AC responsible for cAMP regulating lysosomal acidification, apoptosis, and more.

In contrast to the G protein regulated tmACs, sAC is directly regulated by bicarbonate anions (HCO₃). Due to the ubiquitous presence of carbonic anhydrases (CA), which catalyze the almost instantaneous equilibration of carbon dioxide (CO₂), bicarbonate (HCO₃), and protons, mammalian sAC and its HCO₃-regulated orthologs serve as Nature's physiological CO₂/HCO₃⁻/pHᵢ sensors. By way of HCO₃ regulation of sAC in mammalian cells, CO₂/HCO₃⁻/pHᵢ act as signals regulating a variety of biological functions and physiologies, including sperm activation and motility, intraocular pressure in the eye, ciliary beat frequency in airway, luminal pH in the epididymis and most likely in the kidney, the mitochondrial electron transport chain, activity dependent feeding of neurons in the brain, and glucose stimulated insulin release from 3 cells of the pancreas. In addition to bicarbonate regulation, sAC activity is directly stimulated by Ca²⁺, and it is sensitive to physiological relevant fluctuations in substrate ATP.

Thus, while tmACs respond to signals originating in other cells (i.e., hormones and neuro-transmitters acting via GPCRs), sAC functions as an environmental sensor and an integrator of intracellular signals (HCO₃⁻, ATP, or Ca²⁺).

Due to the important role of sAC in regulating various biological processes, soluble sAC inhibition is an important target for therapy. For a review of sAC biology and uses for sAC inhibitors, see Wiggins et al. “Pharmacological modulation of the CO₂/HCO₃⁻/pH-, calcium, and ATP-sensing soluble adenylyl cyclase”, Pharmacology and Therapeutics, 2018, 190, 173-186, and references cited therein; the entire contents of which is incorporated herein by reference.

### SUMMARY OF THE INVENTION

Due to the varied roles of soluble adenylyl cyclases (sAC) in the body, sAC inhibitors are useful as therapeutic agents, including as contraceptive agents. Soluble adenylyl cyclase (sAC) inhibitors and some uses thereof have been described in, e.g., International PCT Publication WO 2017/190050, published Nov. 2, 2017; the entire contents of which is incorporated herein by reference. Provided herein are new sAC inhibitors which can be used in various methods of treatment (e.g., treatment of ocular conditions (e.g., ocular hypotony, liver diseases (e.g., non-alcoholic steatohepatitis (NASH)), inflammatory diseases, autoimmune diseases (e.g., psoriasis), etc.), and additionally as contraceptive agents (e.g., for male or female contraception). In certain embodiments, the disease or condition that can be treated is a disease or condition typically associated with the activity of a sAC enzyme.

Other sAC inhibitors, and some uses of sAC inhibitors, are described in, e.g., International Application Publication No. WO 2005/070419; International Application Publication No. WO 2006/032541; International Application Publication No. WO 2006/131398; International Application Publication No. WO 2007/107384; International Application Publication No. WO 2009/030725; International Application Publication No. WO 2017/190050; International Application Publication No. WO 2007/010285; and Saalau-Bethell et al., “Crystal structure of human soluble adenylate cyclase reveals a distinct, highly flexible allosteric bicarbonate binding pocket”, ChemMedChem 2014, 9(4), 823-32; “Discovery of LRE1 as a specific and allosteric inhibitor of soluble adenylate cyclase” Nature Chemical Biology 2016 12, 838-844; the entire contents of each of which are incorporated herein by reference.

In one aspect, provided herein are compounds of Formula (I), and pharmaceutically acceptable salts, hydrates, solvates, polymorphs, co-crystals, tautomers, stereoisomers, isotopically labeled derivatives, and prodrugs thereof. Compounds provided herein are soluble adenylyl cyclase (sAC) inhibitors and are therefore useful for the treatment and/or prevention of various diseases and conditions (e.g., ocular conditions (e.g., ocular hypotony), liver diseases (e.g., non-alcoholic steatohepatitis (NASH)), inflammatory diseases, autoimmune diseases (e.g., psoriasis)). In certain embodiments, the disease or condition is associated with the activity of a sAC enzyme. Compounds provided herein are also useful as contraceptive agents (e.g., for male and/or female contraception).

In one aspect, provided herein are compounds of Formula (I):

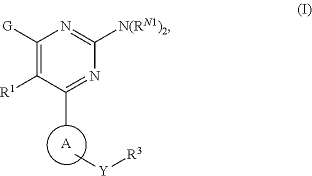

and pharmaceutically acceptable salts, hydrates, solvates, polymorphs, co-crystals, tautomers, stereoisomers, isotopically labeled derivatives, and prodrugs thereof, wherein A, G, R¹, Y, R³, and R^{N1} are as defined herein.

In certain embodiments, a compound of Formula (I) is of Formula (II):

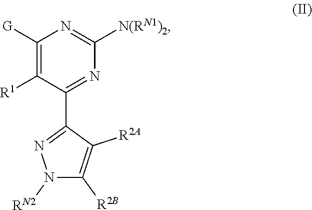

or a pharmaceutically acceptable salt, hydrate, solvate, polymorph, co-crystal, tautomer, stereoisomer, isotopically labeled derivative, or prodrug thereof, wherein G, R¹, R^{2A}, R^{2B}, R^{N1}, and R^{N2} are as defined herein.

In certain embodiments, a compound of Formula (II) is of the following formula:

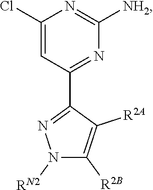

or a pharmaceutically acceptable salt, hydrate, solvate, polymorph, co-crystal, tautomer, stereoisomer, isotopically labeled derivative, or prodrug thereof.

In certain embodiments, for example, a compound provided herein is selected from the group of compounds listed in Table A, vide infra, and pharmaceutically acceptable salts, hydrates, solvates, polymorphs, co-crystals, tautomers, stereoisomers, isotopically labeled derivatives, and prodrugs thereof.

In another aspect, provided herein are pharmaceutical compositions comprising a compound provided herein (e.g., a compound of Formula (I)), or a pharmaceutically acceptable salt, hydrate, solvate, polymorph, co-crystal, tautomer, stereoisomer, isotopically labeled derivative, or prodrug thereof, and a pharmaceutically acceptable carrier or excipient. In certain embodiments, the pharmaceutical composition described herein includes a therapeutically and/or prophylactically effective amount of a compound provided herein (e.g., a compound of Formula (I)), or a pharmaceutically acceptable salt, solvate, hydrate, polymorph, co-crystal, tautomer, stereoisomer, isotopically labeled derivative, or prodrug thereof. The pharmaceutical compositions described herein are useful for treating and/or preventing diseases or conditions in a subject (e.g., ocular conditions (e.g., ocular hypotony), liver diseases (e.g., non-alcoholic steatohepatitis (NASH)), inflammatory diseases, autoimmune diseases (e.g., psoriasis)) in a subject. The pharmaceutical compositions described herein may be useful as contraceptive agents (e.g., for male and/or female contraception).

In another aspect, provided herein are methods for treating and/or preventing a disease or condition in a subject. In certain embodiments, the disease or condition is typically associated with the activity of a sAC enzyme. The methods comprise administering to a subject a compound provided herein (e.g., a compound of Formula (I)), or a pharmaceutically acceptable salt, hydrate, solvate, polymorph, co-crystal, tautomer, stereoisomer, isotopically labeled derivative, or prodrug thereof, or a pharmaceutical composition thereof. In certain embodiments, the disease or condition to be treated or prevented is a disease or condition associated with sAC enzymatic activity. In certain embodiments, the disease or condition is associated with the overexpression, increased activity, and/or aberrant activity of a sAC. In certain embodiments, the disease or condition is associated with normal or baseline level activity of a sAC enzyme. In certain embodiments, the disease or condition is an ocular condition (e.g., ocular hypotony), a liver disease (e.g., non-alcoholic steatohepatitis (NASH)), or an inflammatory or autoimmune disease (e.g., psoriasis).

In another aspect, provided herein are methods for contraception (e.g., male and/or female contraception). The methods comprise administering to a subject (e.g., a male subject in the case of male contraception, or a female subject in the case of female contraception) a compound provided herein (e.g., a compound of Formula (I)), or a pharmaceutically acceptable salt, hydrate, solvate, polymorph, co-crystal, tautomer, stereoisomer, isotopically labeled derivative, or prodrug thereof, or a pharmaceutical composition thereof.

Also provided herein are methods of inhibiting the activity of a soluble adenylyl cyclase (sAC) in a subject or biological sample. The methods comprise administering to a subject, or contacting a biological sample, with a compound provided herein (e.g., a compound of Formula (I)), or a pharmaceutically acceptable salt, hydrate, solvate, polymorph, co-crystal, tautomer, stereoisomer, isotopically labeled derivative, or prodrug thereof, or a pharmaceutical composition thereof.

Also provided here are compounds (e.g., compounds of Formula (I)), and pharmaceutically acceptable salts, hydrates, solvates, polymorphs, co-crystals, tautomers, stereoisomers, isotopically labeled derivatives, and prodrugs thereof, and pharmaceutical compositions thereof, for use in any of the methods described herein. Additionally, provided herein are uses of compounds provided herein (e.g., compounds of Formula (I)), and pharmaceutically acceptable salts, hydrates, solvates, polymorphs, co-crystals, tautomers, stereoisomers, isotopically labeled derivatives, and prodrugs thereof, and pharmaceutical compositions thereof, for the manufacture of medicaments (including for contraception).

In another aspect, provided herein are methods of preparing the compounds provided herein (e.g., compounds of Formula (I)), and pharmaceutically acceptable salts, hydrates, solvates, polymorphs, co-crystals, tautomers, stereoisomers, isotopically labeled derivatives, and prodrugs thereof. Also provided herein are intermediates useful in the preparation of the compounds described herein.

Another aspect of the present disclosure relates to kits comprising a compound (e.g., a compound of Formula (I)), or a pharmaceutically acceptable salt, hydrate, solvate, polymorph, co-crystal, tautomer, stereoisomer, isotopically labeled derivative, or prodrug thereof, or pharmaceutical composition thereof, described herein. The kits described herein may include a single dose or multiple doses of the compound or composition. The provided kits may be useful in a method of the invention (e.g., a method of treating and/or preventing a disease in a subject, a method of contraception). A kit provided herein may further include instructions for using the kit.

The details of certain embodiments of the invention are set forth in the Detailed Description of Certain Embodiments, as described below. Other features, objects, and advantages of the invention will be apparent from the Definitions, Examples, Figures, and Claims.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (i-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋ₛ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂C₁, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified

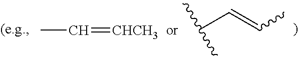

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₄ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”).

Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄).

Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, aziridinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracenyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

The term “unsaturated bond” refers to a double or triple bond. The term “unsaturated” or “partially unsaturated” refers to a moiety that includes at least one double or triple bond. The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, exemplary substituents include, but are not limited to, halogen, —CN, —NO₂, —N₃, —SO₂H, —SO₃H, —OH, —OR^{aa}, —N(R^{bb})₂, —N(R^{bb})₃⁺X⁻, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring; and each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (—CHO), esters (e.g., —CO₂Ra, —C(═O)SR^{aa}, —C(═S)SR^{aa}), amides (e.g., —C(═O)N(R^{bb})₂, —C(═O)NR^{bb}SO₂R^{aa}, —C(═S)N(R^{bb})₂), and imines (e.g., —C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb}) 2)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR″, —C(═O)R″, —CO₂R″, —C(═O)N(R^{bb})₂, —C(═NR^{bb})R^{aa}, —C(═NR^{bb})OR^{aa}, —C(═NR^{bb})N(R^{bb})₂, —S(═O)R″, —SO₂R, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb}) 2)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

### Other Definitions

The following definitions are more general terms used throughout the present application.

As used herein, the term “salt” refers to any and all salts and encompasses pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0.

A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible.

An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₅ alkenyl, C₂-C₅ alkynyl, aryl, C₇-C₁₂ substituted aryl, and C₇-C₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” may refer to a human subject in need of treatment of a disease. In certain embodiments, the subject or patient is a human. In certain embodiments, the subject or patient is a non-human mammal. In certain embodiments, the subject or patient is a dog.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. The disease may also involve an exaggerated response by other immune cells, such as neutrophils. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

An “autoimmune disease” refers to a disease arising from an inappropriate immune response of the body against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), non-alcoholic steatohepatitis (NASH), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

The term “lung disease” or “pulmonary disease” refers to a disease of the lung. Examples of lung diseases include, but are not limited to, primary ciliary dyskinesia, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

A “diabetic condition” refers to diabetes and pre-diabetes. Diabetes refers to a group of metabolic diseases in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger). There are several types of diabetes. Type I diabetes results from the body's failure to produce insulin, and presently requires the person to inject insulin or wear an insulin pump. Type II diabetes results from insulin resistance a condition in which cells fail to use insulin properly, sometimes combined with an absolute insulin deficiency. Gestational diabetes occurs when pregnant women without a previous diagnosis of diabetes develop a high blood glucose level. Other forms of diabetes include congenital diabetes, which is due to genetic defects of insulin secretion, cystic fibrosis-related diabetes, steroid diabetes induced by high doses of glucocorticoids, and several forms of monogenic diabetes, e.g., mature onset diabetes of the young (e.g., MODY 1, 2, 3, 4, 5, 6, 7, 8, 9, or 10). Pre-diabetes indicates a condition that occurs when a person's blood glucose levels are higher than normal but not high enough for a diagnosis of diabetes. All forms of diabetes increase the risk of long-term complications. These typically develop after many years, but may be the first symptom in those who have otherwise not received a diagnosis before that time. The major long-term complications relate to damage to blood vessels. Diabetes doubles the risk of cardiovascular disease and macrovascular diseases such as ischemic heart disease (angina, myocardial infarction), stroke, and peripheral vascular disease. Diabetes also causes microvascular complications, e.g., damage to the small blood vessels. Diabetic retinopathy, which affects blood vessel formation in the retina of the eye, can lead to visual symptoms, reduced vision, and potentially blindness. Diabetic nephropathy, the impact of diabetes on the kidneys, can lead to scarring changes in the kidney tissue, loss of small or progressively larger amounts of protein in the urine, and eventually chronic kidney disease requiring dialysis. Diabetic neuropathy is the impact of diabetes on the nervous system, most commonly causing numbness, tingling and pain in the feet and also increasing the risk of skin damage due to altered sensation. Together with vascular disease in the legs, neuropathy contributes to the risk of diabetes-related foot problems, e.g., diabetic foot ulcers, that can be difficult to treat and occasionally require amputation.

The term “musculoskeletal disease” or “MSD” refers to an injury and/or pain in a subject's joints, ligaments, muscles, nerves, tendons, and structures that support limbs, neck, and back. In certain embodiments, an MSD is a degenerative disease. In certain embodiments, an MSD includes an inflammatory condition. Body parts of a subject that may be associated with MSDs include upper and lower back, neck, shoulders, and extremities (arms, legs, feet, and hands). In certain embodiments, an MSD is a bone disease, such as achondroplasia, acromegaly, bone callus, bone demineralization, bone fracture, bone marrow disease, bone marrow neoplasm, dyskeratosis congenita, leukemia (e.g., hairy cell leukemia, lymphocytic leukemia, myeloid leukemia, Philadelphia chromosome-positive leukemia, plasma cell leukemia, stem cell leukemia), systemic mastocytosis, myelodysplastic syndromes, paroxysmal nocturnal hemoglobinuria, myeloid sarcoma, myeloproliferative disorders, multiple myeloma, polycythemia vera, pearson marrow-pancreas syndrome, bone neoplasm, bone marrow neoplasm, Ewing sarcoma, osteochondroma, osteoclastoma, osteosarcoma, brachydactyly, Camurati-Engelmann syndrome, Craniosynostosis, Crouzon craniofacial dysostosis, dwarfism, achondroplasia, bloom syndrome, Cockayne syndrome, Ellis-van Creveld syndrome, Seckel syndrome, spondyloepiphyseal dysplasia, spondyloepiphyseal dysplasia congenita, Werner syndrome, hyperostosis, osteophyte, Klippel-Trenaunay-Weber syndrome, Marfan syndrome, McCune-Albright syndrome, osteitis, osteoarthritis, osteochondritis, osteochondrodysplasia, Kashin-Beck disease, Leri-Weill dyschondrosteosis, osteochondrosis, osteodystrophy, osteogenesis imperfecta, osteolysis, Gorham-Stout syndrome, osteomalacia, osteomyelitis, osteonecrosis, osteopenia, osteopetrosis, osteoporosis, osteosclerosis, otospondylomegaepiphyseal dysplasia, pachydermoperiostosis, Paget disease of bone, Polydactyly, Meckel syndrome, rickets, Rothmund-Thomson syndrome, Sotos syndrome, spondyloepiphyseal dysplasia, spondyloepiphyseal dysplasia congenita, syndactyly, Apert syndrome, syndactyly type II, or Werner syndrome. In certain embodiments, an MSD is a cartilage disease, such as cartilage neoplasm, osteochondritis, osteochondrodysplasia, Kashin-Beck disease, or Leri-Weill dyschondrosteosis. In certain embodiments, an MSD is hernia, such as intervertebral disk hernia. In certain embodiments, an MSD is a joint disease, such as arthralgia, arthritis (e.g., gout (e.g., Kelley-Seegmiller syndrome, Lesch-Nyhan syndrome), Lyme disease, osteoarthritis, psoriatic arthritis, reactive arthritis, rheumatic fever, rheumatoid arthritis, Felty syndrome, synovitis, Blau syndrome, nail-patella syndrome, spondyloarthropathy, reactive arthritis, Stickler syndrome, synovial membrane disease, synovitis, or Blau syndrome. In certain embodiments, an MSD is Langer-Giedion syndrome. In certain embodiments, an MSD is a muscle disease, such as Barth syndrome, mitochondrial encephalomyopathy, MELAS syndrome, MERRF syndrome, MNGIE syndrome, mitochondrial myopathy, Kearns-Sayre syndrome, myalgia, fibromyalgia, polymyalgia rheumatica, myoma, myositis, dermatomyositis, neuromuscular disease, Kearns-Sayre syndrome, muscular dystrophy, myasthenia, congenital myasthenic syndrome, Lambert-Eaton myasthenic syndrome, myasthenia gravis, myotonia, myotonia congenita, spinal muscular atrophy, tetany, ophthalmoplegia, or rhabdomyolysis. In certain embodiments, an MSD is Proteus syndrome. In certain embodiments, an MSD is a rheumatic diseases, such as arthritis (e.g., gout (e.g., Kelley-Seegmiller syndrome, Lesch-Nyhan lyme disease)), osteoarthritis, psoriatic arthritis, reactive arthritis, rheumatic fever, rheumatoid arthritis, Felty syndrome, synovitis, Blau syndrome, gout (e.g., Kelley-Seegmiller syndrome, Lesch-Nyhan syndrome), polymyalgia rheumatica, rheumatic fever, rheumatic heart disease, or Sjogren syndrome. In certain embodiments, an MSD is Schwartz-Jampel syndrome. In certain embodiments, an MSD is a skeleton disease, such as Leri-Weill dyschondrosteosis, skeleton malformations, Melnick-Needles syndrome, pachydermoperiostosis, Rieger syndrome, spinal column disease, intervertebral disk hernia, scoliosis, spina bifida, spondylitis, ankylosing spondylitis, spondyloarthropathy, reactive arthritis, spondyloepiphyseal dysplasia, spondyloepiphyseal dysplasia congenita, or spondylosis.

An “infectious disease” refers to any disease caused by a pathogen (i.e., pathogenic microorganisms). An infectious disease may be caused by bacteria, viruses, parasites, or fungi. An infectious disease can be a microbial infection. A “microbial infection” refers to an infection with a microorganism, such as a fungus, bacteria or virus. In certain embodiments, the microbial infection is an infection with a fungus, i.e., a fungal infection. In certain embodiments, the microbial infection is an infection with a virus, i.e., a viral infection. In certain embodiments, the microbial infection is an infection with a bacteria, i.e., a bacterial infection. Various microbial infections include, but are not limited to, skin infections, GI infections, urinary tract infections, genito-urinary infections, sepsis, blood infections, and systemic infections. In certain embodiments, the infectious disease is a bacterial infection. In certain embodiments, the infectious disease is a viral infection. In certain embodiments, the infectious disease is a microbial infection.

The term “ocular condition” refers to any disease or condition involving the eye. Examples of ocular conditions include, accommodative dysfunction, amblyopia, astigmatism, blepharitis, cataract, chalazion, color vision deficiency, computer vision syndrome, conjunctivitis, convergence insufficiency, corneal abrasion, crossed eyes, diabetic retinopathy, dry eye, farsightedness, floaters and spots, glaucoma, hordeolum, hyperopia, keratitis, keratoconus, lazy eye, macular degeneration (e.g., age-related macular degeneration (AMD)), migraine with aura, myopia, nearsightedness, nystagmus, ocular allergies, ocular hypertension, ocular migraine visual disturbance, pinquecula, presbyopia, pterygium, ptosis, retinal detachment, retinitis pigmentosa, ocular cancers (e.g., retinoblastoma), strabismus, sty, subconjunctival hemorrhage, and uveitis. In certain embodiments, the ocular condition is associated with low intraocular pressure (IOP).

“Contraception,” also referred to as “birth control,” refers to the prevention of a pregnancy in a subject, e.g., by preventing the fertilization of a female's egg by a male's sperm. “Female contraception” refers to methods wherein the female uses or is administered the contraceptive agent. “Male contraception” refers to methods wherein a male uses or is administered the contraceptive agent.

As used herein, “soluble adenylyl cyclase” (or “sAC”) refers to a specific adenylyl cyclase (AC) enzyme found inside cells in the body. Currently, there are two known, distinct types of adenylyl cyclase enzymes in mammals: bicarbonate-regulated soluble adenylyl cyclase (sAC, ADCY10) and G protein regulated transmembrane adenylyl cyclases (tmACs; ADCY1-9). Cyclic AMP (cAMP) is a messenger molecule that is produced from ATP by adenylyl cyclases (ACs), and degraded by catabolizing phosphodiesterases (PDEs). Soluble adenylyl cyclase (sAC) is an independent source of cAMP in intracellular microdomains and is found distributed through the cytoplasm and in cellular organelles, including inside the nucleus and the mitochondrial matrix. Cyclic AMP (cAMP), and by extension sAC, is implicated in a variety of physiological processes. The sequence of human sAC can be found, e.g., under GenBank Accession Number AF176813.

As used herein the term “inhibit” or “inhibition” in the context of enzymes, for example, in the context of sAC, refers to a reduction in the activity of the enzyme. In some embodiments, the term refers to a reduction of the level of enzyme activity (e.g., sAC) to a level that is statistically significantly lower than an initial level, which may, for example, be a baseline level of enzyme activity. In some embodiments, the term refers to a reduction of the level of enzyme activity (e.g., sAC activity) to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

These and other exemplary substituents are described in more detail in the Detailed Description, Examples, and Claims. The invention is not intended to be limited in any manner by the above exemplary listing of substituents.

### BRIEF DESCRIPTION OF THE DRAWINGS

The accompanying drawings, which constitute a part of this specification, illustrate several embodiments of the invention and together with the description, serve to explain the principles of the invention.

FIG. 1. Chemical structure of exemplary sAC inhibitor Example 1. FIG. 2. Chemical structure of exemplary sAC inhibitor Example 133.

FIG. 3. Intraocular pressure (IOP) study. The sAC inhibitor, Example 1, dose dependently elevates IOP in wild type (WT) C57B1/6 mouse eyes one hour post ip injection.

FIG. 4 shows the type 17 inflammatory response, measured by ear thickness from the left ear daily, in wild type C57B1/6 male mice treated with vehicle (blue circle) or Imiquimod (purple triangle) and Adcy10^{-/-} C57B1/6 male mice with vehicle (red square) or Imiquimod (yellow triangle). Repeated measures ANOVA (legend *'s), post-hoc Sidak (*'s comparing purple to yellow symbols). * p<0.05, ** p<0.01, *** p<0.001. FIG. 5 shows clinical (left panel) and histologic (right panel) images from mice in (A) on day seven. #, parakeratosis. *, granular cell layer.

FIGS. 6A-6B. FIG. 6A shows the gating strategy used for in vivo (upper panels) and in vitro (lower panels) analysis of CD45+, CD4+, IL17+ T cells. FIG. 6B shows a comparison of the percentage of CD45+, CD4+(left panel) and CD45+, CD8+(right panel) between C57B1/6 wild type (WT) and Adcy10−/− (KO) mice.

FIGS. 7A-7B. FIG. 7A shows a representative flow cytometry analysis of CD45+, CD4+, IL17+ T cells in C57B1/6 wild type (WT) and Adcy10−/− (KO) mice following vehicle and Imiquimod treatment for six days to the back. FIG. 7B shows a compendium (n=11 mice) of flow cytometry analysis of CD45+, CD4+, IL17+ T cells in C57B1/6 wild type and Adcy10−/− mice following vehicle (−) or Imiquimod (IMQ, +) treatment for six days to the back. Experiment performed three times. Experimental days with matched data point shape. Data points average of triplicate determinations. Data presented as fold over vehicle. ANOVA, sidak post-hoc. **, p<0.01.

FIGS. 8A-8B. Quantitative RT-PCR analysis of type 17 inflammatory (FIG. 8A) cytokine gene and (FIG. 8B) keratinocyte gene expression in the skin of wild type C57B1/6 male mice treated with vehicle (blue symbols) or Imiquimod (purple symbols) and Adcy10−/− C57B1/6 male mice treated with vehicle (red symbols) or Imiquimod (yellow symbols). Each symbol represents data obtained from one mouse. Triplicate determinations. Matched symbol shapes (circles, squares, and triangles) represents data obtained on the same day. Representative of an experiment performed three times. ANOVA, sidak post-hoc. *, p<0.05; **, p<0.01; ***, p<0.001. (FIG. 8A) n≥6. (FIG. 8B) n≥3.

FIGS. 9A-9B. FIG. 9A shows IL-17 secretion, measured by ELISA, from CD4+ T cells derived from C57B1/6 wild type and Adcy10−/− mice following four days in the presence of anti-CD3/CD28 antibodies with (+) and without (−) Th17 polarizing conditions (Th17 Cyt) IL1b/IL-6/IL-23. Data presented as fold over baseline. n=8. FIG. 9B, the left panel, is a representative flow cytometry analysis; and the right panel is a compendium of the percentage of CD45+, CD4+, IL17+ T cells from C57B1/6 wild type (WT) and Adcy10−/− (KO) mice following culture with anti-CD3/CD28 antibodies with (+) and without (Negative, −) IL1b/IL-6/IL-23 (Th17 Cyt) cytokines. Each symbol represents data obtained from one mouse. Triplicate determinations. Matched symbol shapes (circles, squares, and triangles) represents data obtained on the same day. Data points average of triplicate determinations. ANOVA, sidak post-hoc. **, p<0.01; ***, p<0.001; ****, p<0.0001

FIGS. 10A-10B. FIG. 10A shows a type 17 inflammatory response, measured by ear thickness of both ears, in wild type C57B1/6 male mice treated with Imiquimod daily for 6 days followed by continued daily Imiquimod treatment and either twice a day treatment with vehicle (black circles), sAC inhibitor (LRE1, 3%, red squares), or Clobetasol (0.05%, green triangles) for 5 days. Repeated measures ANOVA, post-hoc Sidak (#'s vehicle to drug treatment, p<0.0001 for all points). **** p<0.0001. FIG. JOB shows a quantitative RT-PCR analysis of Il17a and Il17f expression in skin of the experiment as described in FIG. bOA. Each symbol represents data obtained from each mouse. Triplicate determinations. Representative of an experiment performed three times. ANOVA, sidak post-hoc. *, p<0.05; **, p<0.01.

FIG. 11 shows a type 17 inflammatory response, measured by ear thickness of both ears, in wild type C57B1/6 male mice treated with Imiquimod daily for 6 days followed by continued daily Imiquimod treatment and either twice a day treatment with vehicle (blue circles), sAC inhibitor (LRE1, 3%, red squares), Example 1 (1.5%, green triangles), or Clobetasol (0.05%, purple triangles) for 5 days. Repeated measures ANOVA, post-hoc Sidak (#'s vehicle to drug treatment, p<0.0001 for all points). **** p<00.0001.

FIG. 12 shows sAC inhibition by Example 1 prevents bicarbonate-induced changes in flagellar beating pattern of mouse sperm. Representative images of flagellar waveform of mouse sperm in the absence or presence of 5 μM Example 1 after stimulation with 25 mM NaHCO₃. Superimposed color-coded frames taken every 5 ms, illustrating one flagellar beat cycle; scale bar: 15 μm.

FIG. 13 shows sAC inhibition by Example 1 prevents bicarbonate-induced changes in flagellar beating pattern of human sperm. Representative images of flagellar waveform of human sperm in the absence or presence of 0.2 μM Example 1 after stimulation with 25 mM NaHCO₃. Superimposed color-coded frames taken every 5 ms, illustrating one flagellar beat cycle; scale bar: 15 μm.

FIG. 14 shows sAC inhibition by Example 1 blocks in vitro fertilization. Rate of two-cell stage oocytes after incubation of mouse oocytes with capacitated wild-type sperm in the absence or presence of 5 or 50 μM Example 1; mean+SEM (n=5), numbers indicate total number of oocytes from three independent experiments. Differences between conditions were analyzed using one-way ANOVA compared to respective DMSO-treated control, *P<0.05, **P<0.01, ***P<0.001, ****P<0.0001.

FIG. 15. Imiquimod was applied to induce Th17 inflammation. At the same time, vehicle, LRE-1 and Example 1 were applied. Example 1 and LRE-1 both reduce the inflammation in the ears as measured by ear calipers. LRE-1 has less of an effect than Example 1. N=5.

FIG. 16. Imiquimod was applied for one week to induce inflammation in all mice. Mice were then randomized (N=5) into groups to continue receiving imiquimod but also receiving either vehicle, Example 1, Example 69, or clobetasol. Application of Example 1 or Example 69 led to a 50% reduction in inflammation over 4 days.

FIG. 17. Imiquimod was applied for one week to induce inflammation in all mice. Mice were then randomized (N=5) into groups to continue receiving imiquimod but also receiving either vehicle, Example 1, or Example 133. Application of Example 1 or Example 133 led to a dramatic reduction in inflammation relative to vehicle which continued to increase in inflammation over 6 days.

FIGS. 18-20B show potent sAC inhibitors with long retention times. FIG. 18 shows the concentration-response curves of Example 1 (IC₅₀=159 nM) and Example 133 (IC₅₀=3 nM) on in vitro adenylyl cyclase activity of purified recombinant human sAC protein in the presence of 1 mM ATP, 2 mM Ca²⁺, 4 mM Mg²⁺, and 40 mM HCO₃, normalized to the respective DMSO-treated control; mean±SEM (n≥6). FIG. 19 shows the concentration-response curves of Example 1 (IC₅₀=102 nM) and Example 133 (IC₅₀=7 nM) on sAC-dependent cAMP accumulation in sAC-overexpressing 4/4 cells grown in media containing 10% FBS treated with 500 μM IBMX for 5 minutes, normalized to the respective DMSO-treated control; mean±SEM (n≥6). FIGS. 20A and 20B show the parallel kinetics of Example 1 (FIG. 20A) or Example 133 (FIG. 20B) binding to immobilized sAC protein measured using surface plasmon resonance. Representative traces of experiments repeated at least 3 times showing binding kinetics of different concentrations of inhibitor along with best fits using a 1:1 binding model (black lines). Example 1: kₒₙ=2.3×10⁵/ns, k_{off}=55.8×10³/s; Example 133: kₒₙ=2.4×10⁵/ms, k_{off}=0.3×10⁻³/s.

FIGS. 21A-21H show sAC inhibitors inhibit essential functions in sperm, and a sAC inhibitor with long retention time inhibits sperm functions even after dilution. FIGS. 21A and 21C show the intracellular cAMP levels in mouse (FIG. 21A) and human (FIG. 21C) sperm incubated in non-capacitating (striped bars) or capacitating media in the absence or presence of 5 μM Example 1 or 10 nM Example 133. Shown are cAMP levels measured after 12 minute incubations; mean+SEM (n≥8). FIGS. 21B and 21D show intracellular cAMP levels in mouse (FIG. 21C) and human (FIG. 21D) sperm following dilution into inhibitor-free media. After preincubation (5 minutes) in 5 μM Example 1 or 10 nM Example 133, sperm were diluted (1:10) in inhibitor-free non-capacitating (striped bars) or capacitating media (solid bars). Shown are cAMP levels measured 12 minutes after dilution; mean+SEM (n≥5). Only the inhibitor with long retention time, Example 133, inhibits capacitation induced cAMP rise in diluted sperm. FIGS. 21E and 21F show the mean flagellar beat frequency along the length of the tail (arc length, μm) of mouse (FIG. 21E) and human (FIG. 21F) sperm in the absence or presence of 5 μM Example 1 or 10 nM Example 133 before and after stimulation with 25 mM NaHCO₃. Solid lines indicate the time-averaged values, dotted lines the SEM, n=3, ≥60 individual sperm from 3 different mice or 3 different human donors. FIGS. 21G and 21H show the acrosome reaction in mouse (FIG. 21G) sperm evoked by 50 heat-solubilized zona pellucida (striped bars) and human (FIG. 21H) sperm evoked by 10 μM progesterone (striped bars) after incubation for 90 minutes (mouse) or 180 minutes (human) in capacitating media in the absence or presence of 5 μM Example 1 or 10 nM Example 133 in the absence or presence of 5 mM db-cAMP/500 μM IBMX; mean+SEM (n≥5). Differences between conditions were analyzed using one-way ANOVA compared to DMSO-treated capacitated control *P<0.05, **P<0.01, ***P<0.001, ****P<0.0001.

FIGS. 22A-22B show a single dose of systemically delivered sAC inhibitor with long retention time blocks essential functions in epididymal sperm after dilution ex vivo. FIG. 22A shows the relative cAMP increase due to incubation in capacitating conditions of epididymal mouse sperm isolated at the indicated times following injection (i.p.) with vehicle (DMSO:PEG 400:PBS 1:4:5), 50 mg/kg Example 1 or 50 mg/kg Example 133. Isolated sperm were minimally diluted (solid bars) or 1:200 diluted (striped bars) into inhibitor-free capacitating or non-capacitating media, and cAMP was measured 12 minutes after dilution into capacitating or non-capacitating media. Values shown are cAMP levels in capacitating sperm relative to cAMP levels in non-capacitated sperm from the same mouse; mean+SEM (n≥8). FIG. 22B shows the progressive motility of epididymal mouse sperm isolated at the indicated time points post injection (i.p.) with vehicle (Gray bar), 50 mg/kg Example 1 (light blue bar) or 50 mg/kg Example 133 (purple bars). Isolated sperm were diluted 1:200 in inhibitor-free non-capacitating media, and percent motility assessed by CASA. For sperm isolated from Example 133-injected males one hour post-injection, motility was also assessed in the presence of 5 mM db-cAMP/500 μM IBMX (striped bar). Differences between conditions were analyzed using two-tailed, unpaired t-test comparing sperm isolated from inhibitor-injected mice to sperm isolated from vehicle-injected mice at the respective time point, *P<0.05, **P<0.01, ***P<0.001, ****P<0.0001.

FIG. 22C shows mouse sperm motility is blocked after systemic exposure with sAC inhibitors. Representative motility tracks of sperm isolated from male mice at the indicated time points post injection (i.p.) with vehicle, 50 mg/kg Example 1 or 50 mg/kg Example 133 diluted 1:20 in inhibitor-free non-capacitating media. Motility of sperm isolated after 1 h from Example 133-injected males in the presence of 5 mM db-cAMP/500 μM IBMX.

FIGS. 23A-23D show long residence time sAC inhibitors delay human sperm hyperactivation after dilution into inhibitor free media. We first determined the dose-response relationship for each sAC inhibitor in FIGS. 23A and 23B, which show the percentage of human sperm displaying hyperactivated motility in non-capacitating (light grey bars) or capacitating media in the absence (dark grey bar) or presence (colored bars) of indicated concentrations of Example 1, FIG. 23A (light blue bars), or Example 133, FIG. 23B (dark blue bars). For the highest concentration of inhibitor, motility was also assessed in the presence of 5 mM db-cAMP/500 μM IBMX (striped bars); mean+SEM (n≥5). FIGS. 23C and 23D show the percentage of human sperm displaying hyperactivated motility at the indicated time points after substantial dilution into inhibitor-free capacitating media following preincubation in non-capacitating media in the presence of 10 μM Example 1, FIG. 23C, or 100 nM Example 3, FIG. 23D. Fully inhibited controls show percent hyperactivation of human sperm diluted into capacitating media containing the same concentration of inhibitor used for preincubation (light blue or light purple). Fully capacitated controls show percent hyperactivation of human sperm in capacitating media in the presence of vehicle alone (dark grey), and non-capacitated controls show percent hyperactivation of human sperm in non-capacitating media in the presence of vehicle alone (light grey); mean+SEM (n≥5). Differences between conditions were analyzed using one-way ANOVA compared to the DMSO-treated capacitated control (FIGS. 23A and 23B), *P<0.05, **P<0.01, ***P<0.001, ****P<0.0001.

FIGS. 24A-24F show mouse sperm tyrosine phosphorylation is blocked after systemic exposure with sAC inhibitors. FIGS. 24A, 24C, and 24E show phosphorylation of tyrosine residues of mouse sperm isolated from mice one hour post injection (i.p.) with vehicle, FIG. 24A, 50 mg/kg Example 1, FIG. 24C, or 50 mg/kg Example 133, FIG. 24E, after the indicated dilutions between 1:20 through 1:1000 in inhibitor-free capacitating media. Shown are representative Western Blots. FIGS. 24B, 24D, and 24F show quantitation of tyrosine residues of mouse sperm isolated from mice one hour post injection (i.p.) with vehicle, FIG. 24B, 50 mg/kg Example 1, FIG. 24D, or 50 mg/kg Example 133, FIG. 24F, after the indicated dilutions between 1:20 through 1:1000 in inhibitor-free capacitating media. Tyrosine phosphorylation patterns were normalized to non-capacitated sperm (striped bars) from vehicle-injected controls; mean+SEM (n≥6).

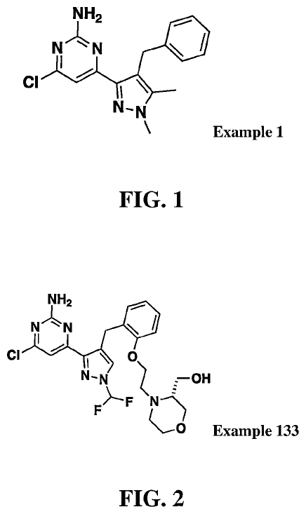

### DETAILED DESCRIPTION OF CERTAIN EMBODIMENTS

Provided herein are soluble adenylyl cyclase (sAC) inhibitors and uses thereof. In one aspect, provided herein are compounds of Formula (I), and pharmaceutically acceptable salts, hydrates, solvates, polymorphs, co-crystals, tautomers, stereoisomers, isotopically labeled derivatives, and prodrugs thereof, and pharmaceutical compositions thereof. The compounds provided herein are soluble adenylyl cyclase (sAC) inhibitors and are therefore useful for the treatment and/or prevention of various diseases and conditions, such as ones associated with the activity of a sAC enzyme (e.g., ocular conditions (e.g., ocular hypotony), liver diseases (e.g., non-alcoholic steatohepatitis (NASH)), inflammatory diseases, autoimmune diseases (e.g., psoriasis)). Compounds provided herein are also useful as contraceptive agents (e.g., for male and/or female contraception). Therefore, in another aspect, provided herein are methods of using the compounds and pharmaceutical compositions provided herein. In other aspects, provided herein are kits comprising compounds and pharmaceutical compositions described herein, methods of synthesizing compounds provided herein, and intermediates useful in the synthesis of compounds provided herein.

### Compounds

Provided herein are compounds of Formula (I):

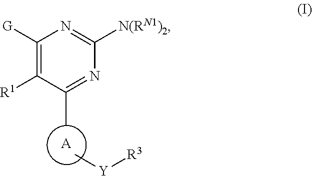

and pharmaceutically acceptable salts, hydrates, solvates, polymorphs, co-crystals, tautomers, stereoisomers, isotopically labeled derivatives, and prodrugs thereof, wherein:
G is halogen, —CN, optionally substituted alkyl, or optionally substituted acyl;
R¹ is hydrogen, halogen, optionally substituted alkyl, or optionally substituted acyl;
A is an optionally substituted monocyclic heteroaryl ring comprising at least 1 nitrogen atom;
Y is a bond, optionally substituted alkylene, optionally substituted heteroalkylene, —O—, —NR^{N}—, —S—, —S(═O)—, or —SO₂—;
R³ is optionally substituted carbocyclyl, optionally substituted heterocyclyl, optionally substituted aryl, or optionally substituted heteroaryl;
each instance of R^{N1} is independently hydrogen, optionally substituted alkyl, optionally substituted acyl, or a nitrogen protecting group, or optionally two R^{N1} are taken together with the intervening atoms to form optionally substituted heterocyclyl or optionally substituted heteroaryl;
provided that when G is not halogen, -(A)-Y—R³ is of the formula:

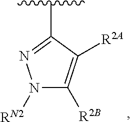

wherein:
R^{2A} and R^{2B} are independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, —SR^{S}, or —Y—R³;
provided that one of R^{2A} and R^{2B} is —Y—R³;
R^{N2} is hydrogen, optionally substituted alkyl, optionally substituted acyl, or a nitrogen protecting group;
each instance of R^{N} is independently hydrogen, optionally substituted alkyl, optionally substituted acyl, or a nitrogen protecting group, or optionally two R^{N} are taken together with the intervening atoms to form optionally substituted heterocyclyl or optionally substituted heteroaryl;
each instance of R^{O} is independently hydrogen, optionally substituted alkyl, optionally substituted acyl, or an oxygen protecting group; and
each instance of R^{S} is independently hydrogen, optionally substituted alkyl, optionally substituted acyl, or a sulfur protecting group.

In certain embodiments, ring A is an optionally substituted pyrazole ring. In certain embodiments, a compound of Formula (I) is of Formula (II):

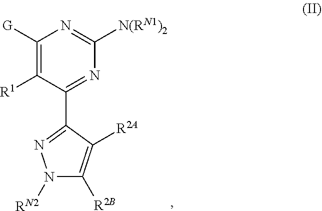

or a pharmaceutically acceptable salt, hydrate, solvate, polymorph, co-crystal, tautomer, stereoisomer, isotopically labeled derivative, or prodrug thereof, wherein one of R^{2A} and R^{2B} is —Y—R³.

In certain embodiments, G is halogen. In certain embodiments, G is —Cl. In certain embodiments, a compound of Formula (II) is of the formula:

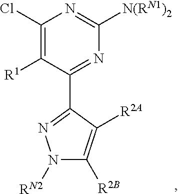

or a pharmaceutically acceptable salt, hydrate, solvate, polymorph, co-crystal, tautomer, stereoisomer, isotopically labeled derivative, or prodrug thereof.

In certain embodiments, R¹ is hydrogen. In certain embodiments, a compound of Formula (II) is of Formula (III):

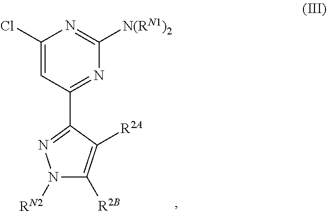

or a pharmaceutically acceptable salt, hydrate, solvate, polymorph, co-crystal, tautomer, stereoisomer, isotopically labeled derivative, or prodrug thereof.

In certain embodiments, a compound of Formula (III) is of the formula:

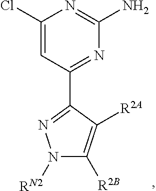

or a pharmaceutically acceptable salt, hydrate, solvate, polymorph, co-crystal, tautomer, stereoisomer, isotopically labeled derivative, or prodrug thereof.

In certain embodiments, R^{2A} is —Y—R³. In certain embodiments, a compound of Formula (III) is of Formula (IV):

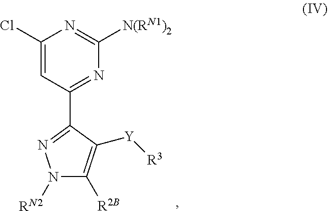

or a pharmaceutically acceptable salt, hydrate, solvate, polymorph, co-crystal, tautomer, stereoisomer, isotopically labeled derivative, or prodrug thereof.

In certain embodiments, a compound of Formula (IV) is of the formula:

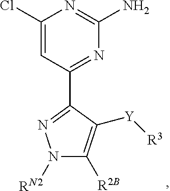

or a pharmaceutically acceptable salt, hydrate, solvate, polymorph, co-crystal, tautomer, stereoisomer, isotopically labeled derivative, or prodrug thereof.

In certain embodiments, R³ is optionally substituted phenyl. In certain embodiments, a compound of Formula (IV) is of Formula (V):

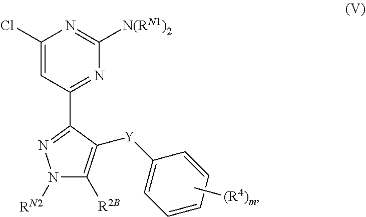

or a pharmaceutically acceptable salt, hydrate, solvate, polymorph, co-crystal, tautomer, stereoisomer, isotopically labeled derivative, or prodrug thereof, wherein:
each instance of R⁴ is independently halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, or —SR^{S}; and
m is 0, 1, 2, 3, 4, or 5.

In certain embodiments, a compound of Formula (V) is of the formula:

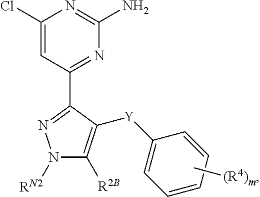

or a pharmaceutically acceptable salt, hydrate, solvate, polymorph, co-crystal, tautomer, stereoisomer, isotopically labeled derivative, or prodrug thereof.

In certain embodiments, Y is optionally substituted C₁₋₃ alkylene. In certain embodiments, Y is optionally substituted methylene. In certain embodiments, a compound of Formula (V) is of Formula (VI):

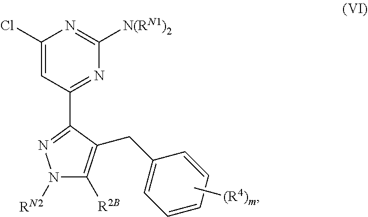

or a pharmaceutically acceptable salt, hydrate, solvate, polymorph, co-crystal, tautomer, stereoisomer, isotopically labeled derivative, or prodrug thereof.

In certain embodiments, both R^{N1} are hydrogen. In certain embodiments, a compound of Formula (VI) is of the formula:

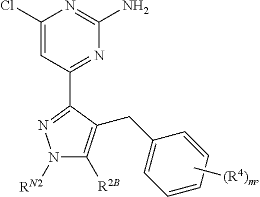

or a pharmaceutically acceptable salt, hydrate, solvate, polymorph, co-crystal, tautomer, stereoisomer, isotopically labeled derivative, or prodrug thereof.

In certain embodiments, m is 1. In certain embodiments, a compound of Formula (VI) is of the formula:

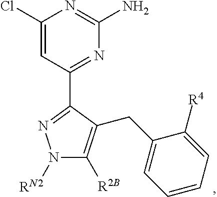

or a pharmaceutically acceptable salt, hydrate, solvate, polymorph, co-crystal, tautomer, stereoisomer, isotopically labeled derivative, or prodrug thereof.

In certain embodiments, at least one instance of R⁴ is —Z—R⁵. In certain embodiments, a compound of Formula (VI) is of Formula (VII):

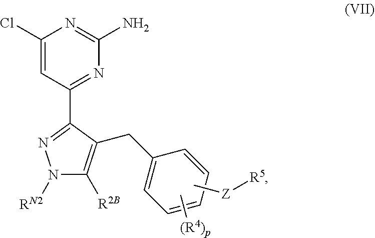

or a pharmaceutically acceptable salt, hydrate, solvate, polymorph, co-crystal, tautomer, stereoisomer, isotopically labeled derivative, or prodrug thereof, wherein:
Z is a bond, optionally substituted alkylene, optionally substituted heteroalkylene, or optionally substituted acylene;
R⁵ is optionally substituted heterocyclyl, optionally substituted heteroaryl, —N(R^{N})₂, or —OR^{O}; and
p is 0, 1, 2, 3, or 4.

In certain embodiments, a compound of Formula (VII) is of the formula:

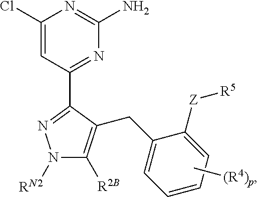

or a pharmaceutically acceptable salt, hydrate, solvate, polymorph, co-crystal, tautomer, stereoisomer, isotopically labeled derivative, or prodrug thereof.

In certain embodiments, R^{2B} is hydrogen. In certain embodiments, a compound of Formula (VII) is of the formula:

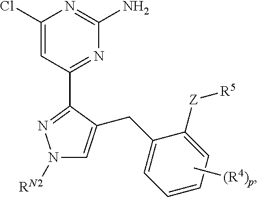

or a pharmaceutically acceptable sa t, hydrate, solvate, polymorph, co-crystal, tautomer, stereoisomer, isotopically labeled derivative, or prodrug thereof.

In certain embodiments, a compound of Formula (VII) is of the formula:

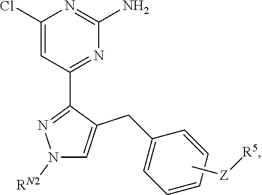

or a pharmaceutically acceptable salt, hydrate, solvate, polymorph, co-crystal, tautomer, stereoisomer, isotopically labeled derivative, or prodrug thereof.

In certain embodiments, a compound of Formula (VII) is of the formula:

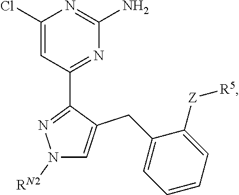

or a pharmaceutically acceptable salt, hydrate, solvate, polymorph, co-crystal, tautomer, stereoisomer, isotopically labeled derivative, or prodrug thereof.

In certain embodiments, a compound of Formula (I) is selected from the compounds listed in Table A, vide infra, and pharmaceutically acceptable salts, hydrates, solvates, polymorphs, co-crystals, tautomers, stereoisomers, isotopically labeled derivatives, and prodrugs thereof.

In certain embodiments, a compound of Formula (I) is selected from the group consisting of:

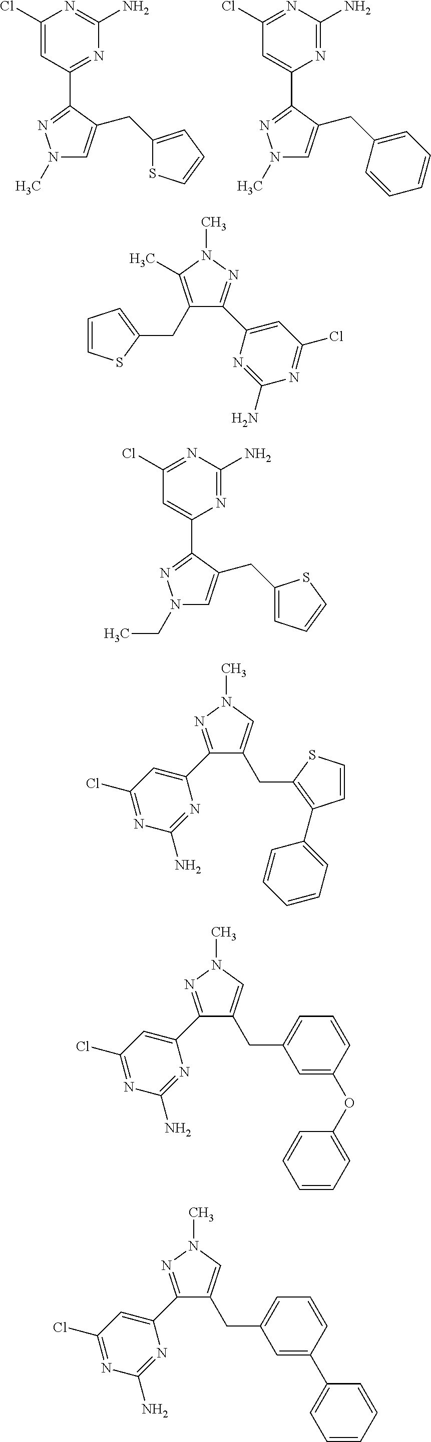

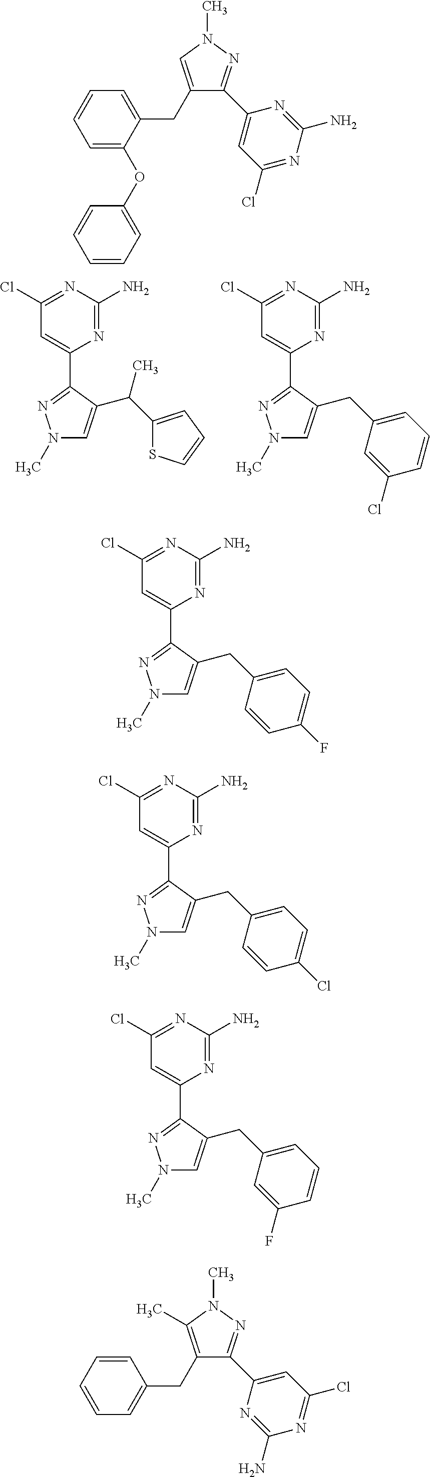

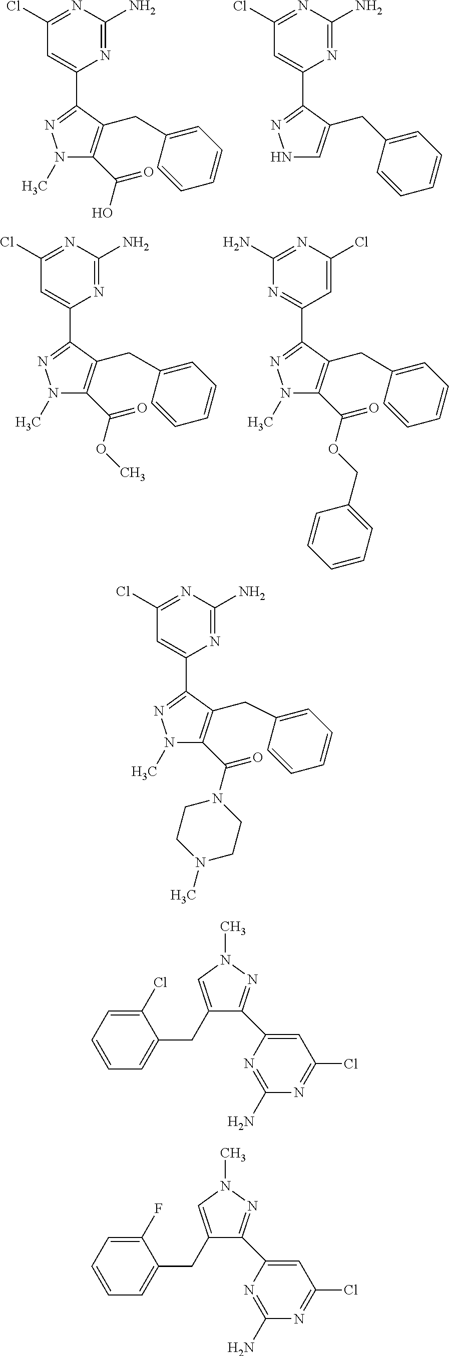

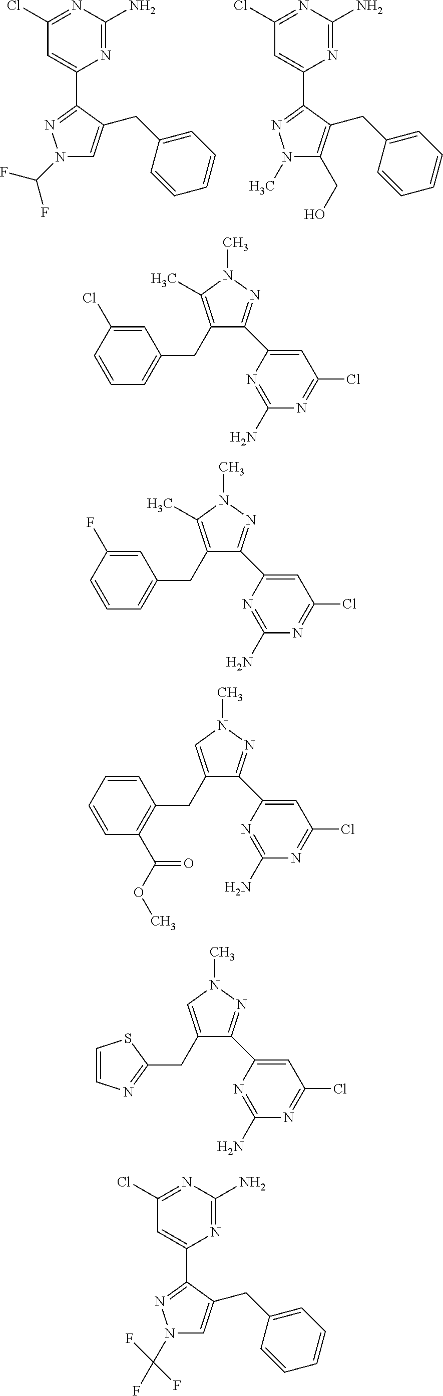

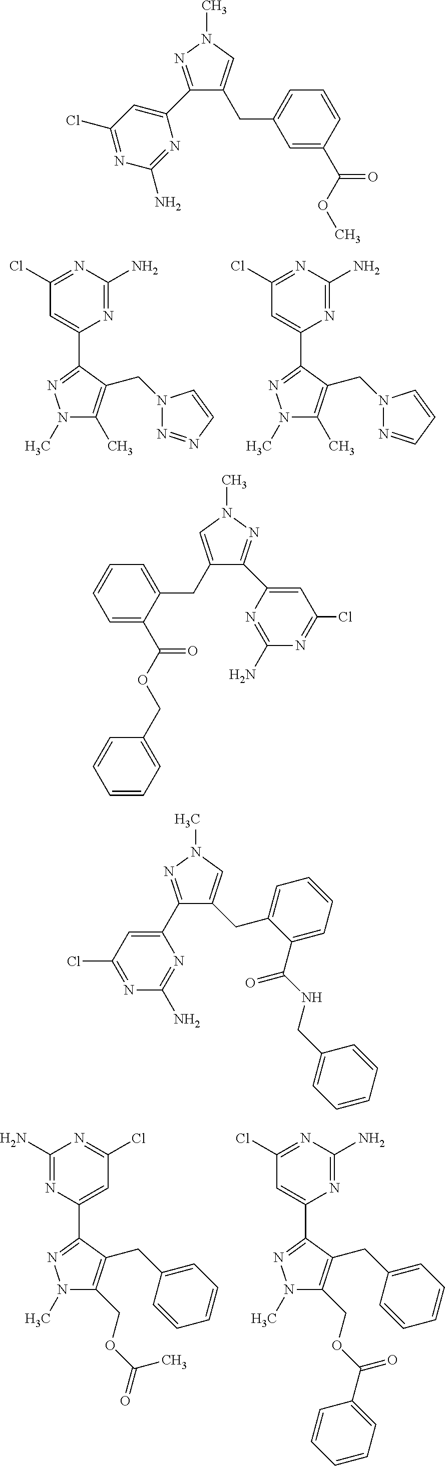

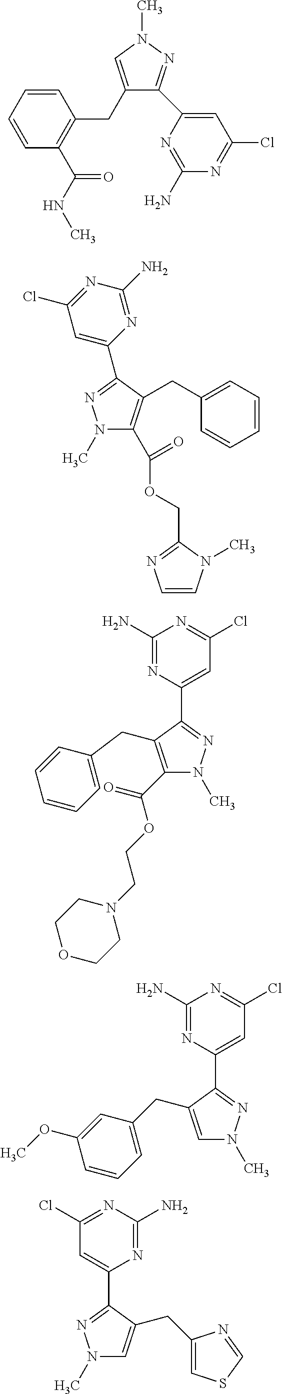

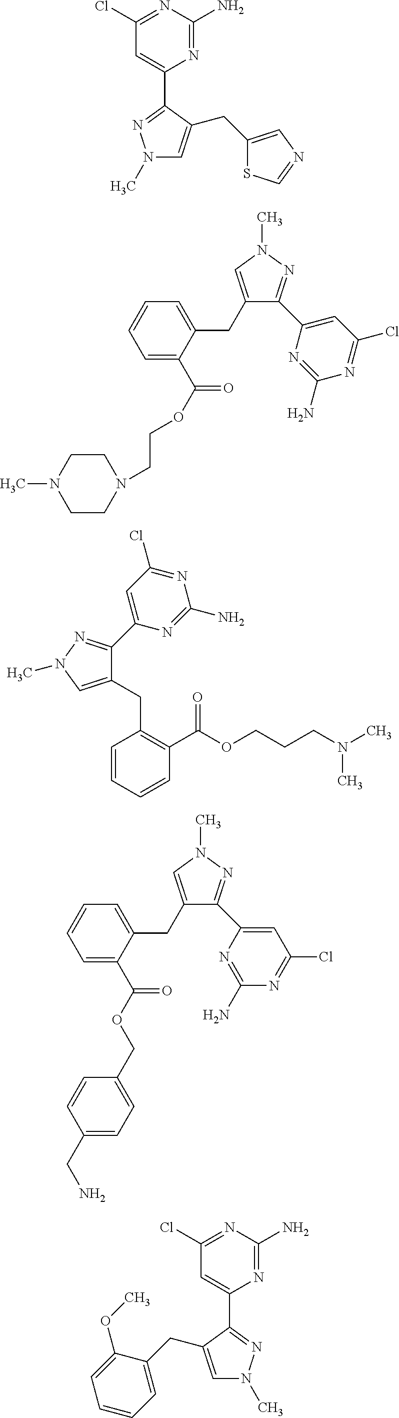

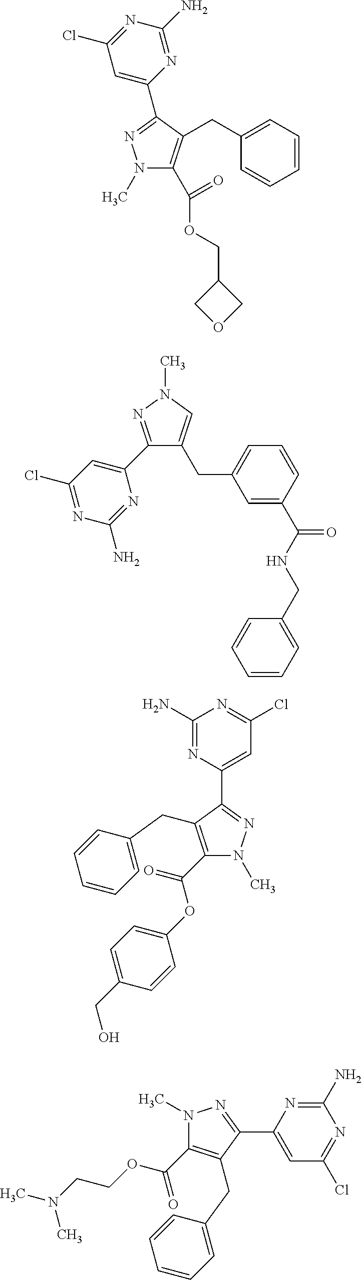

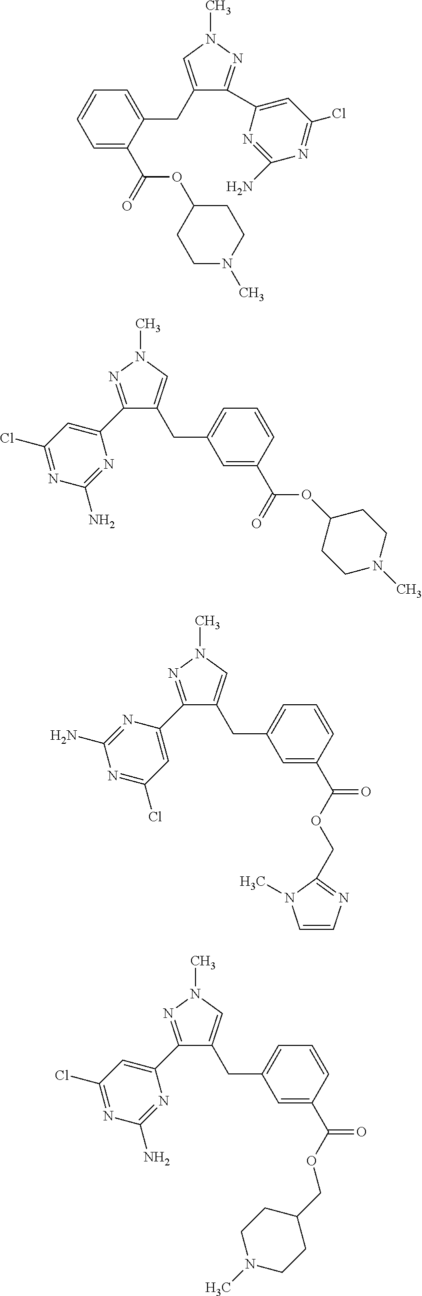 [SMILES: CN1CCC(OC(=O)c2ccccc2Cc2cn(C)nc2-c2cc(Cl)nc(N)n2)CC1 ; CN1CCC(OC(=O)c2cccc(Cc3cn(C)nc3-c3cc(Cl)nc(N)n3)c2)CC1 ; Cn1cc(Cc2cccc(C(=O)OCc3nccn3C)c2)c(-c2cc(Cl)nc(N)n2)n1 ; CN1CCC(COC(=O)c2cccc(Cc3cn(C)nc3-c3cc(Cl)nc(N)n3)c2)CC1]

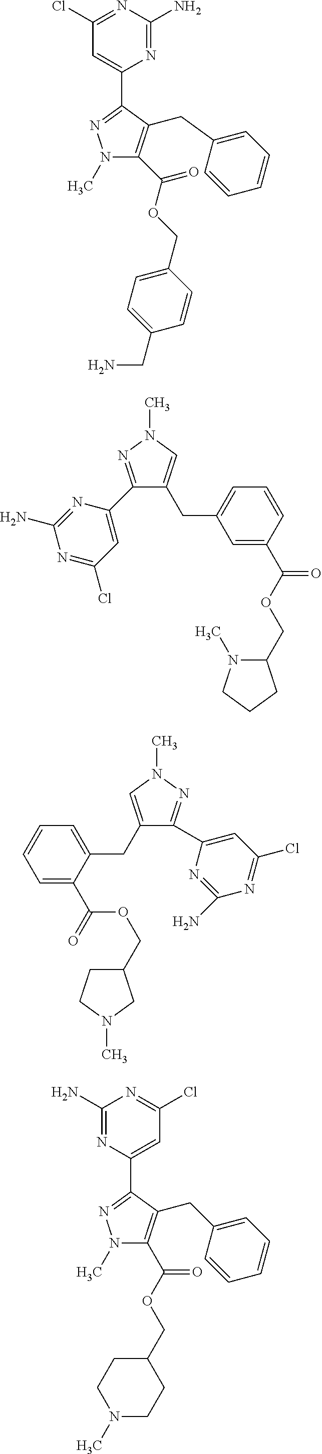

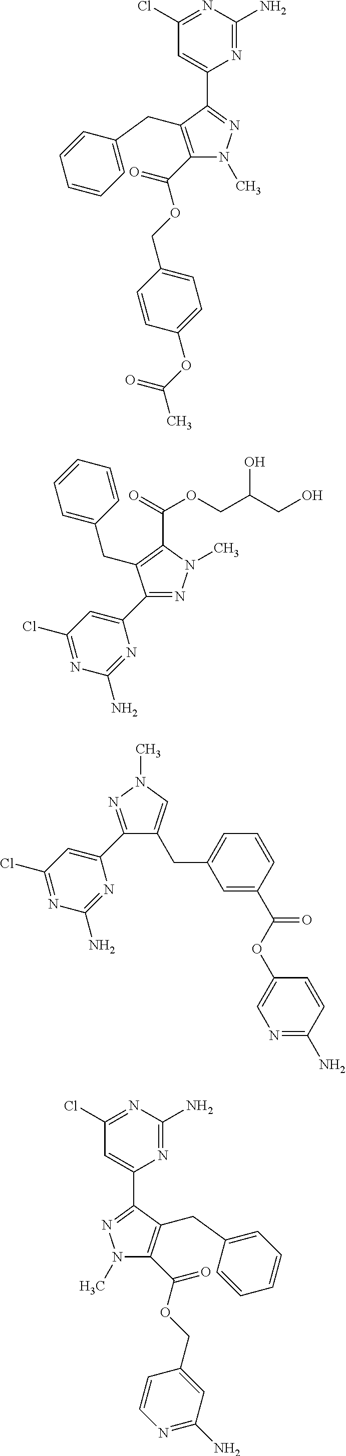 [SMILES: CC(=O)Oc1ccc(COC(=O)c2c(Cc3ccccc3)c(-c3cc(Cl)nc(N)n3)nn2C)cc1 ; Cn1nc(-c2cc(Cl)nc(N)n2)c(Cc2ccccc2)c1C(=O)OCC(O)CO ; Cn1cc(Cc2cccc(C(=O)Oc3ccc(N)nc3)c2)c(-c2cc(Cl)nc(N)n2)n1 ; Cn1nc(-c2cc(Cl)nc(N)n2)c(Cc2ccccc2)c1C(=O)OCc1ccnc(N)c1]

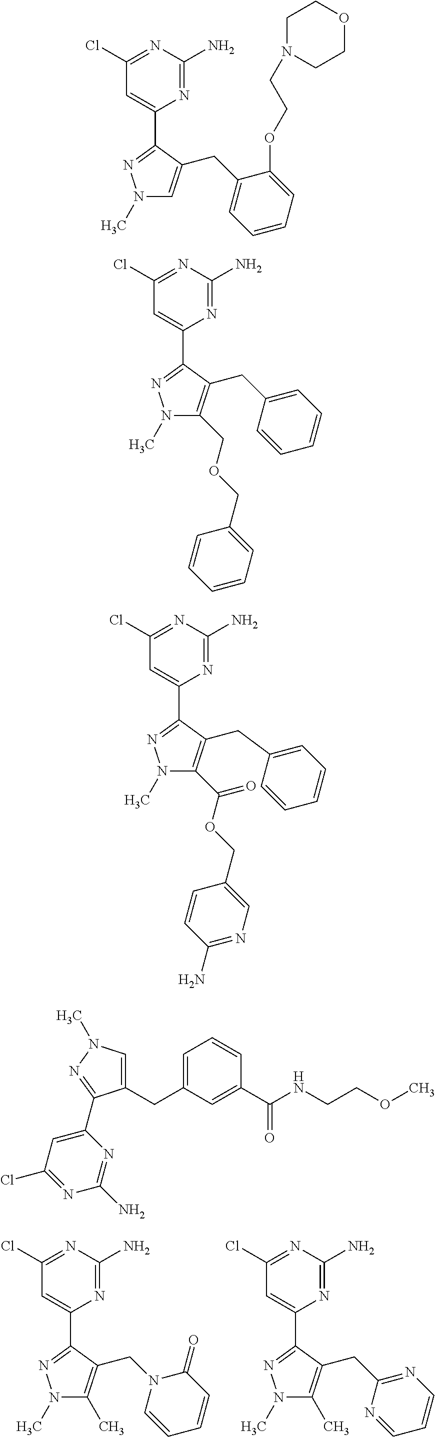

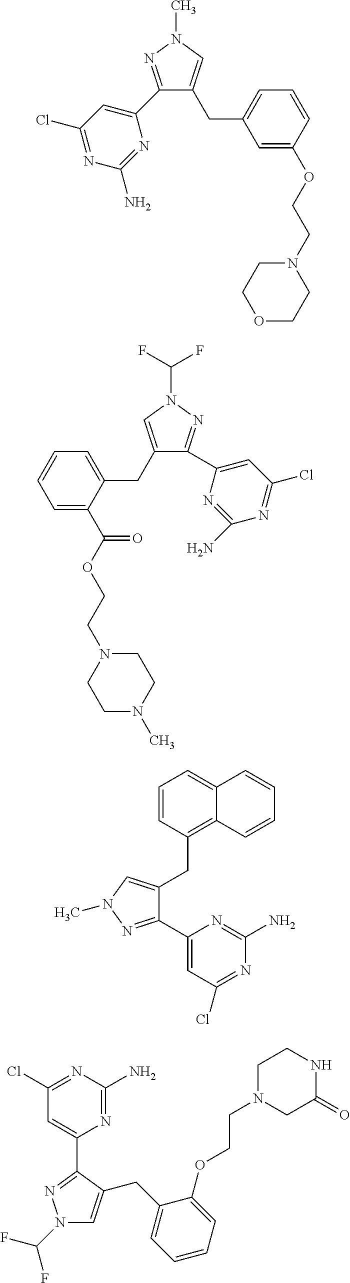

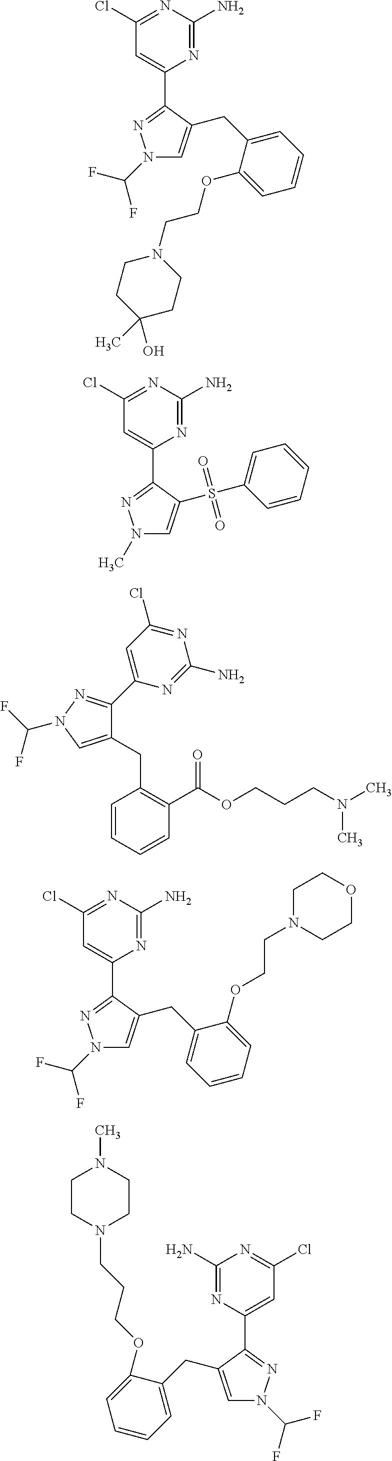

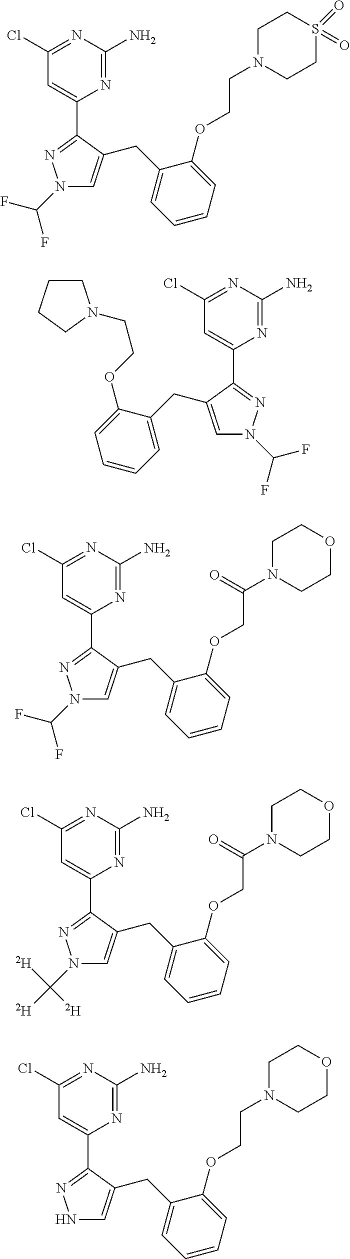

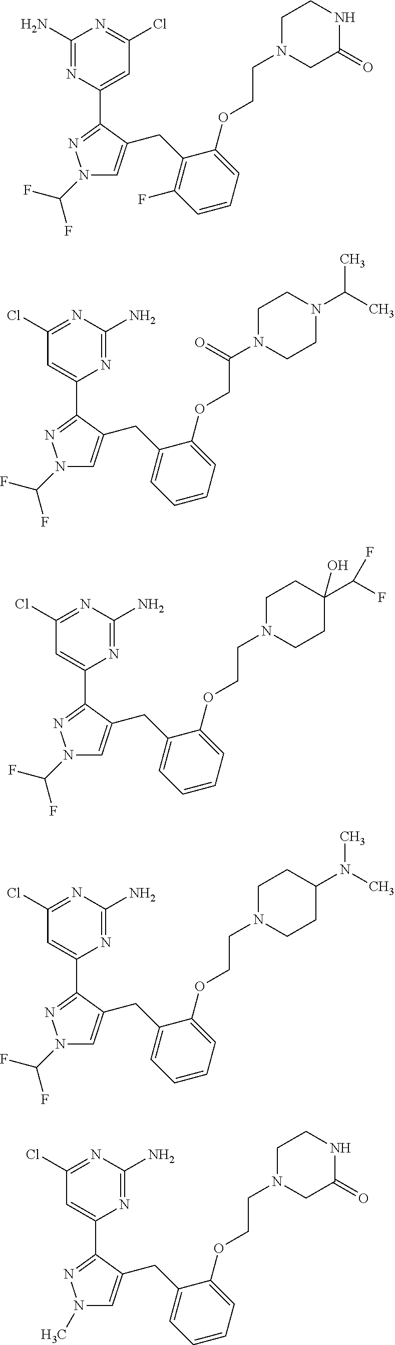 [SMILES: Nc1nc(Cl)cc(-c2nn(C(F)F)cc2Cc2c(F)cccc2OCCN2CCNC(=O)C2)n1 ; CC(C)N1CCN(C(=O)COc2ccccc2Cc2cn(C(F)F)nc2-c2cc(Cl)nc(N)n2)CC1 ; Nc1nc(Cl)cc(-c2nn(C(F)F)cc2Cc2ccccc2OCCN2CCC(O)(C(F)F)CC2)n1 ; CN(C)C1CCN(CCOc2ccccc2Cc2cn(C(F)F)nc2-c2cc(Cl)nc(N)n2)CC1 ; Cn1cc(Cc2ccccc2OCCN2CCNC(=O)C2)c(-c2cc(Cl)nc(N)n2)n1]

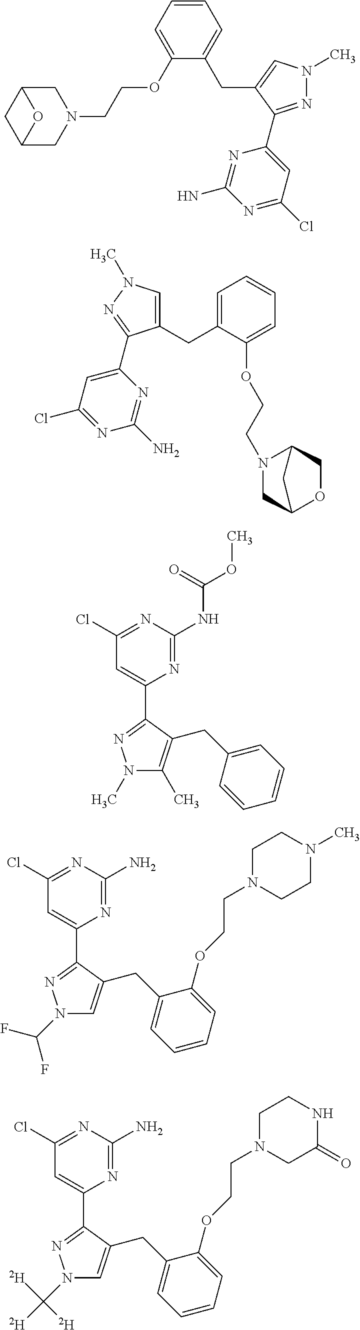

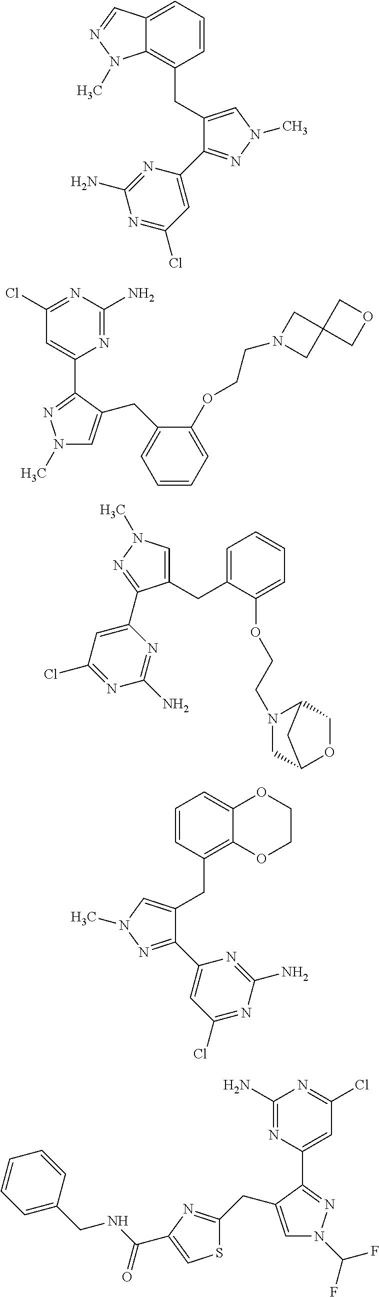

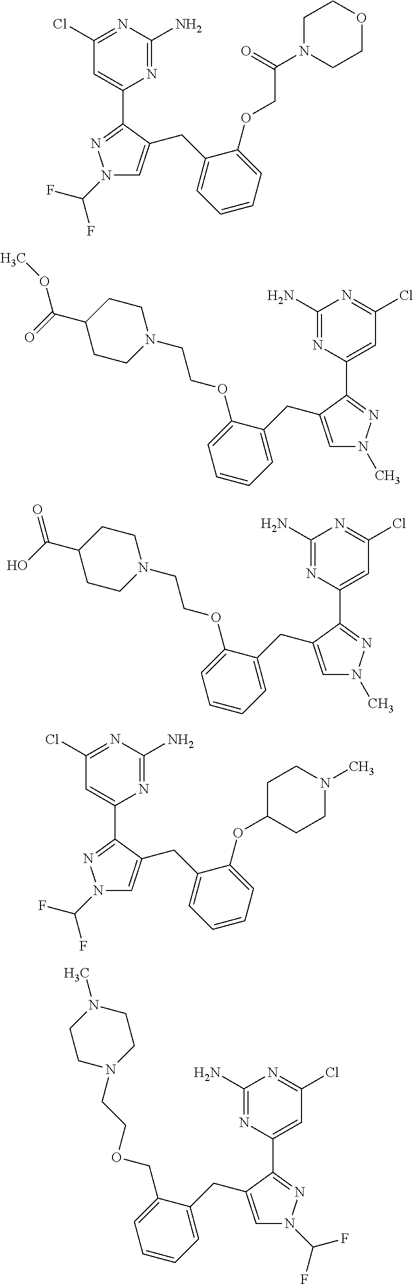

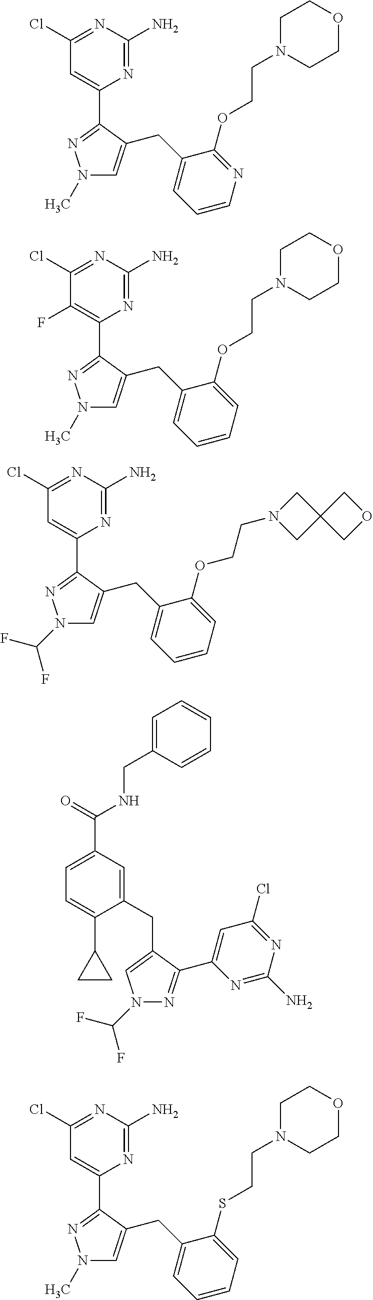

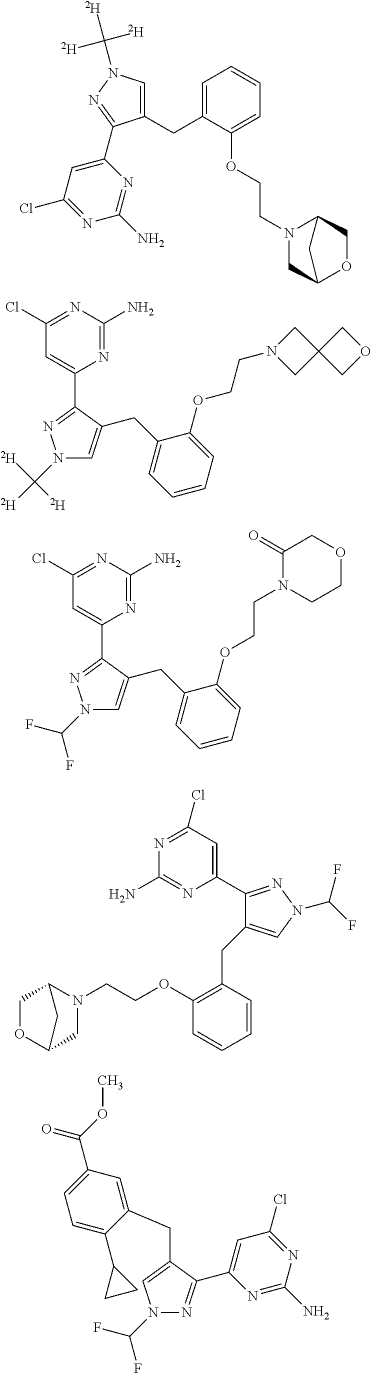

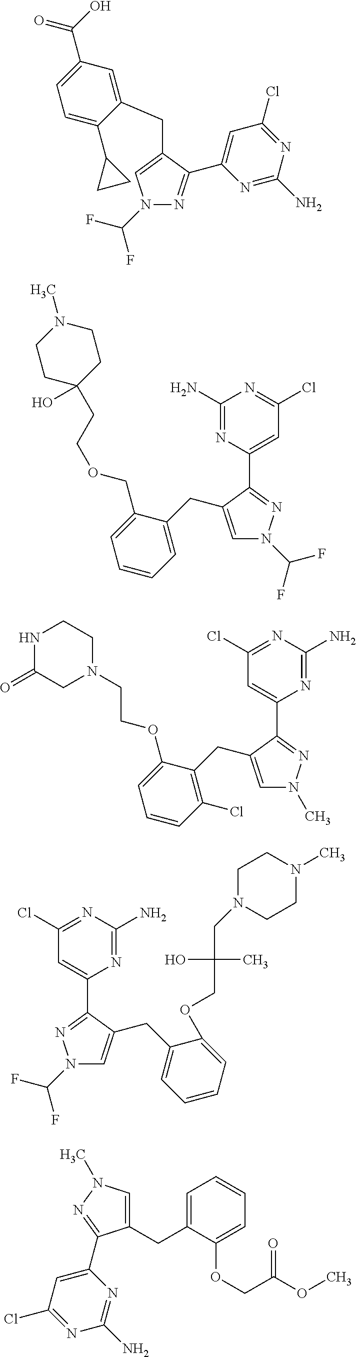

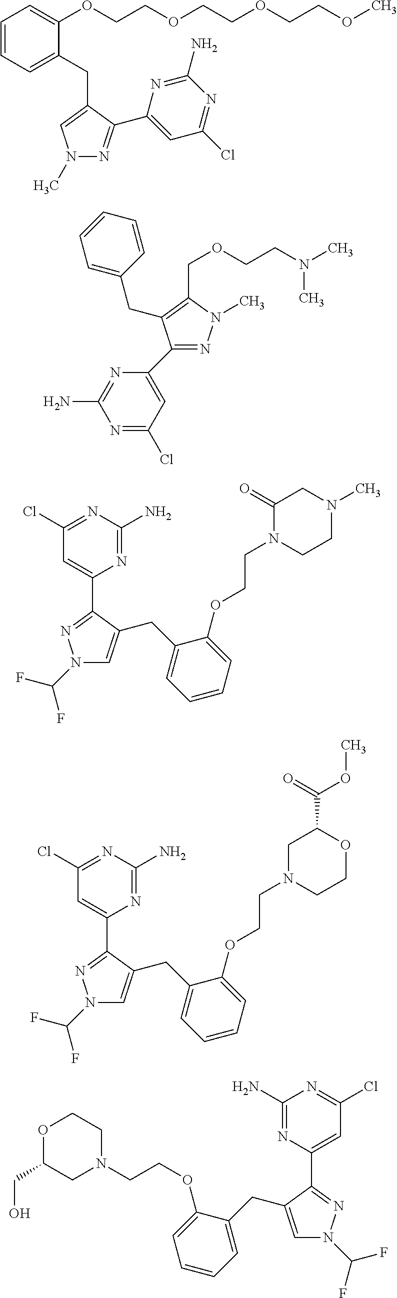 [SMILES: COCCOCCOCCOc1ccccc1Cc1cn(C)nc1-c1cc(Cl)nc(N)n1 ; CN(C)CCOCc1c(Cc2ccccc2)c(-c2cc(Cl)nc(N)n2)nn1C ; CN1CCN(CCOc2ccccc2Cc2cn(C(F)F)nc2-c2cc(Cl)nc(N)n2)C(=O)C1 ; COC(=O)[C@H]1CN(CCOc2ccccc2Cc2cn(C(F)F)nc2-c2cc(Cl)nc(N)n2)CCO1 ; Nc1nc(Cl)cc(-c2nn(C(F)F)cc2Cc2ccccc2OCCN2CCO[C@@H](CO)C2)n1]

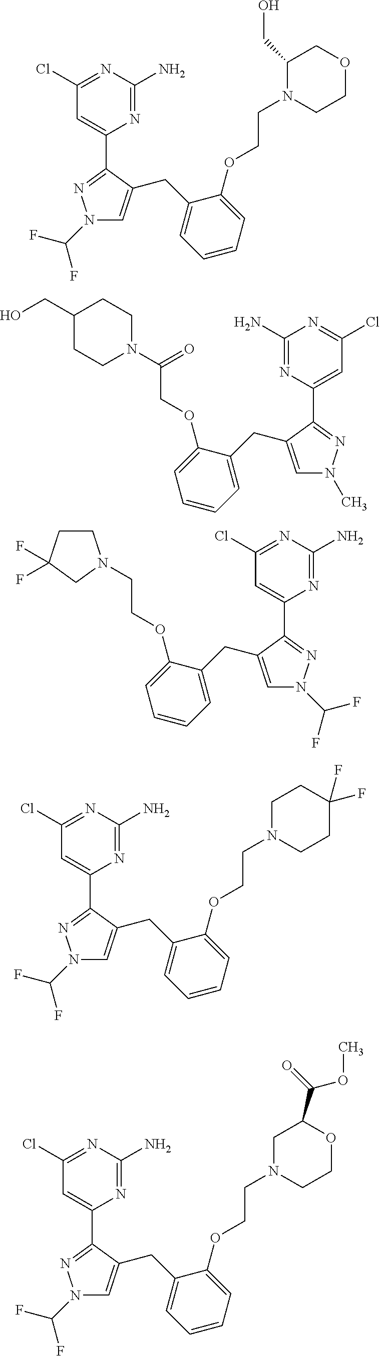

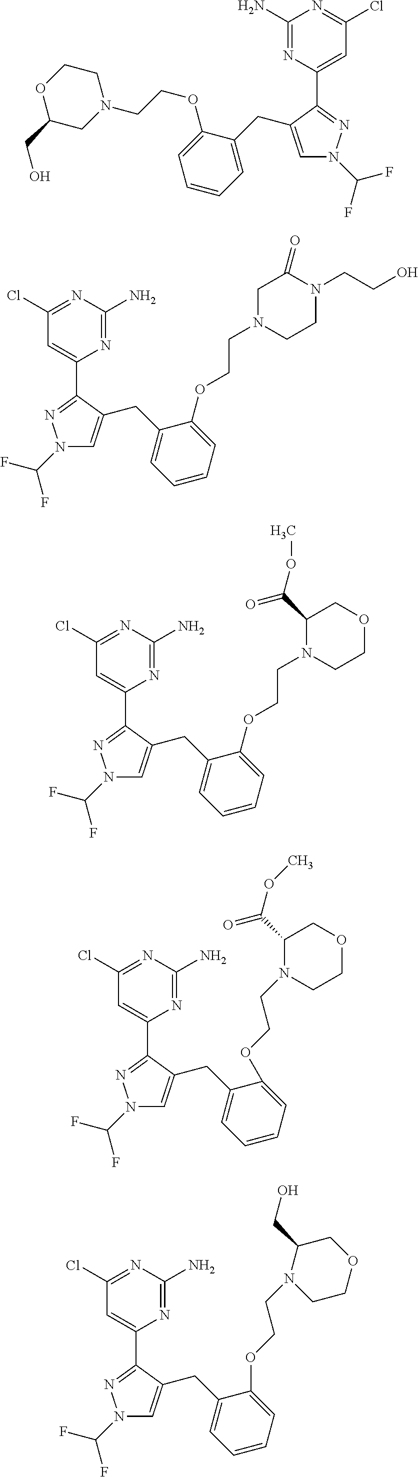 [SMILES: Nc1nc(Cl)cc(-c2nn(C(F)F)cc2Cc2ccccc2OCCN2CCO[C@H](CO)C2)n1 ; Nc1nc(Cl)cc(-c2nn(C(F)F)cc2Cc2ccccc2OCCN2CCN(CCO)C(=O)C2)n1 ; COC(=O)[C@H]1COCCN1CCOc1ccccc1Cc1cn(C(F)F)nc1-c1cc(Cl)nc(N)n1 ; COC(=O)[C@@H]1COCCN1CCOc1ccccc1Cc1cn(C(F)F)nc1-c1cc(Cl)nc(N)n1 ; Nc1nc(Cl)cc(-c2nn(C(F)F)cc2Cc2ccccc2OCCN2CCOC[C@@H]2CO)n1]

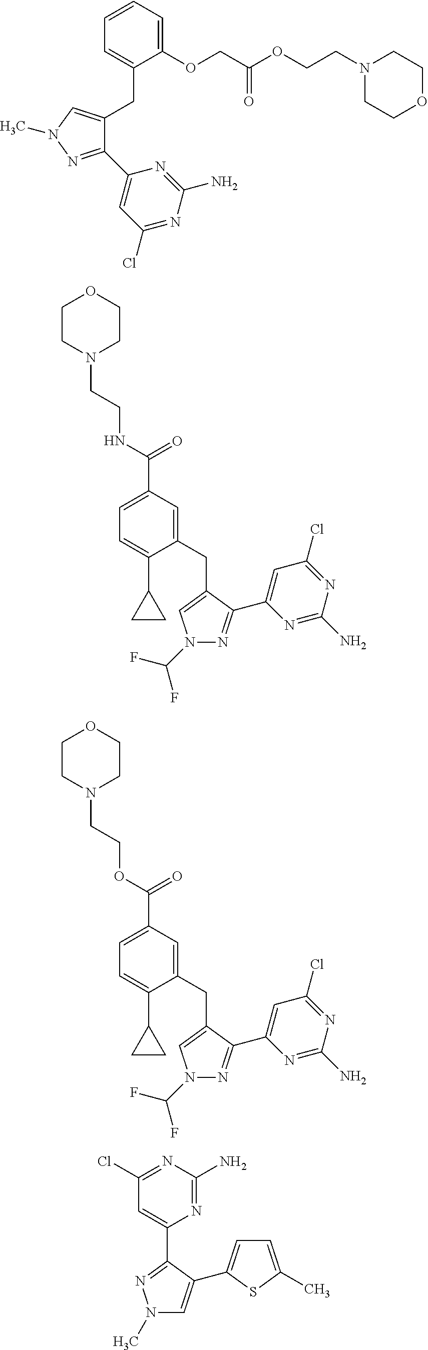

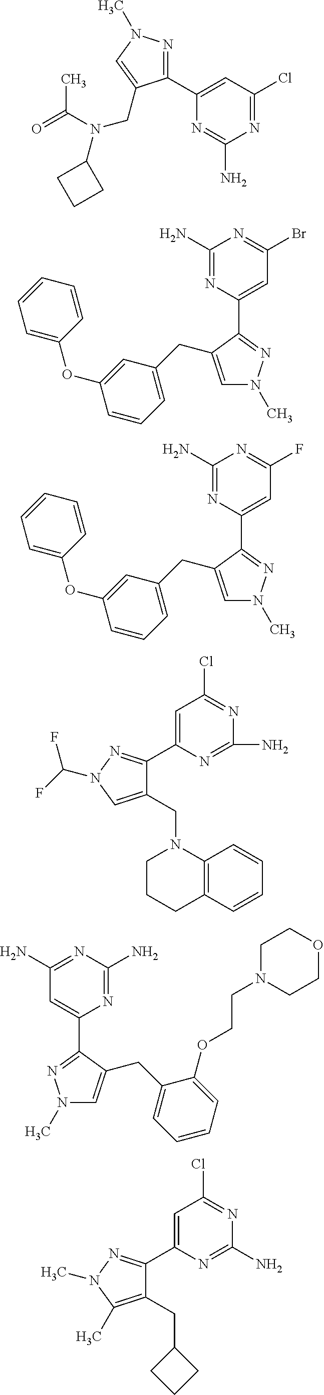

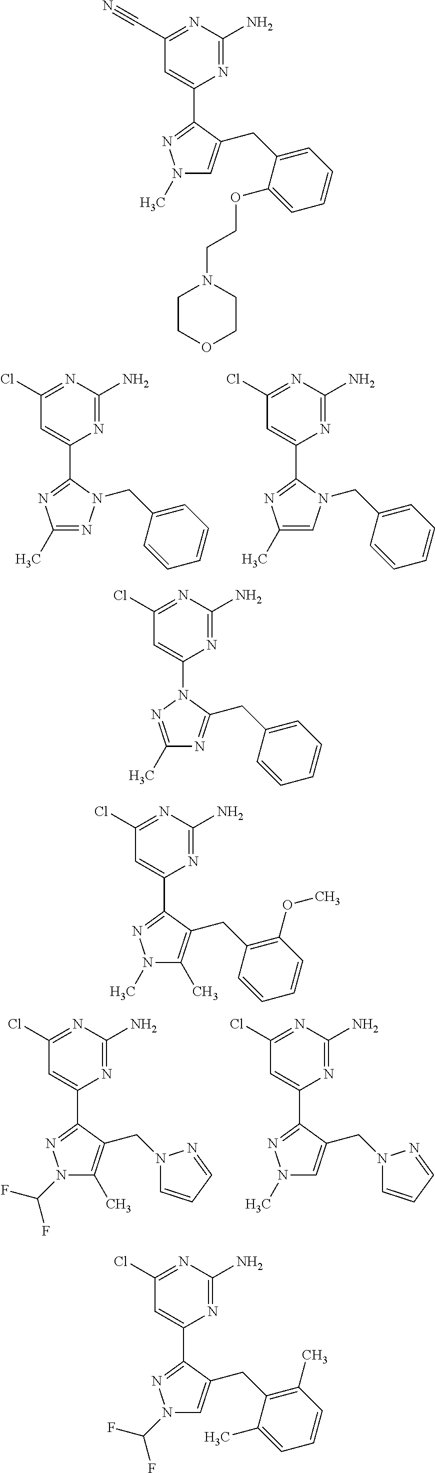

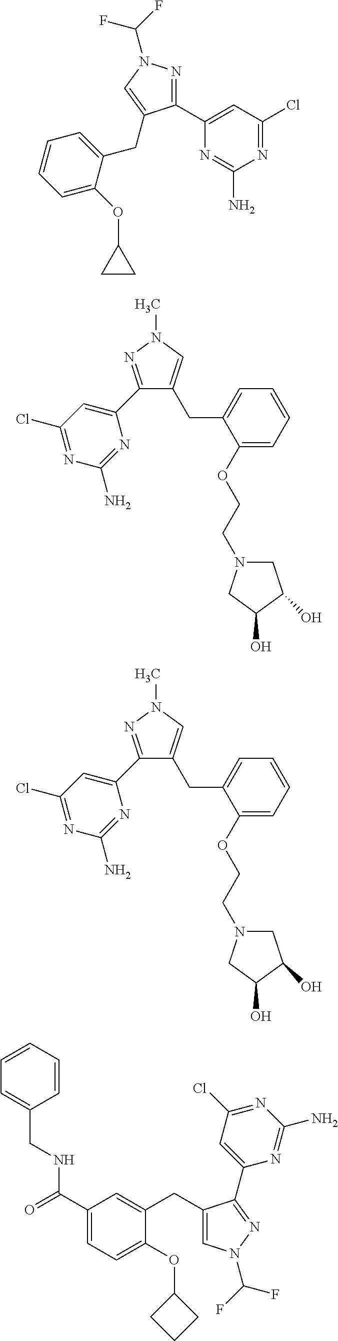

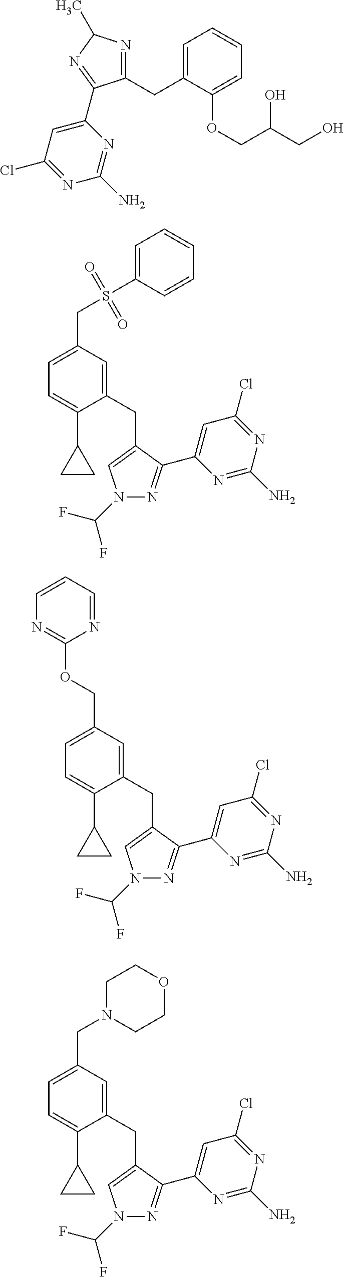

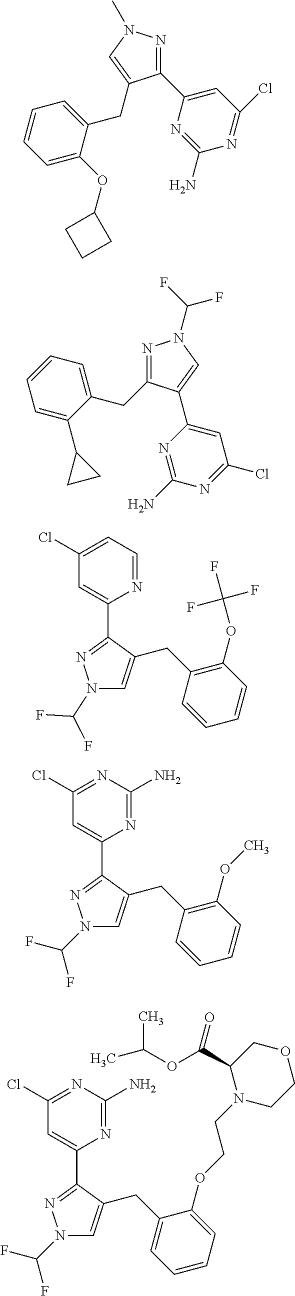

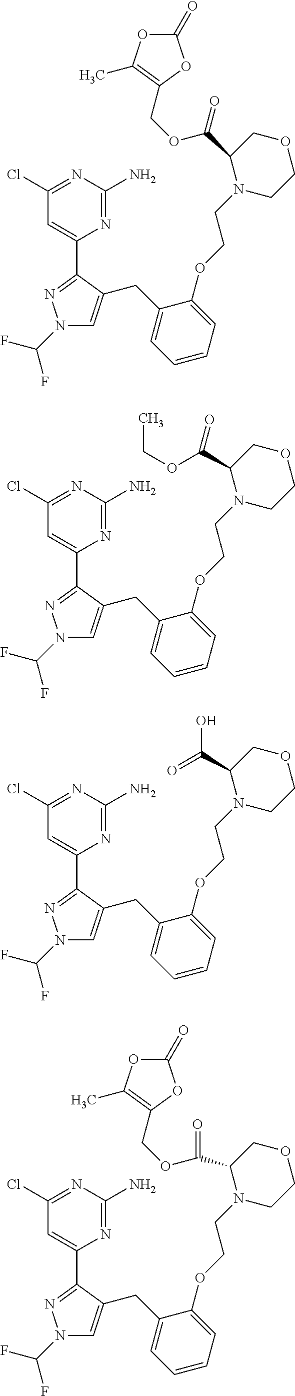 [SMILES: Cc1oc(=O)oc1COC(=O)[C@H]1COCCN1CCOc1ccccc1Cc1cn(C(F)F)nc1-c1cc(Cl)nc(N)n1 ; CCOC(=O)[C@H]1COCCN1CCOc1ccccc1Cc1cn(C(F)F)nc1-c1cc(Cl)nc(N)n1 ; Nc1nc(Cl)cc(-c2nn(C(F)F)cc2Cc2ccccc2OCCN2CCOC[C@@H]2C(=O)O)n1 ; Cc1oc(=O)oc1COC(=O)[C@@H]1COCCN1CCOc1ccccc1Cc1cn(C(F)F)nc1-c1cc(Cl)nc(N)n1]

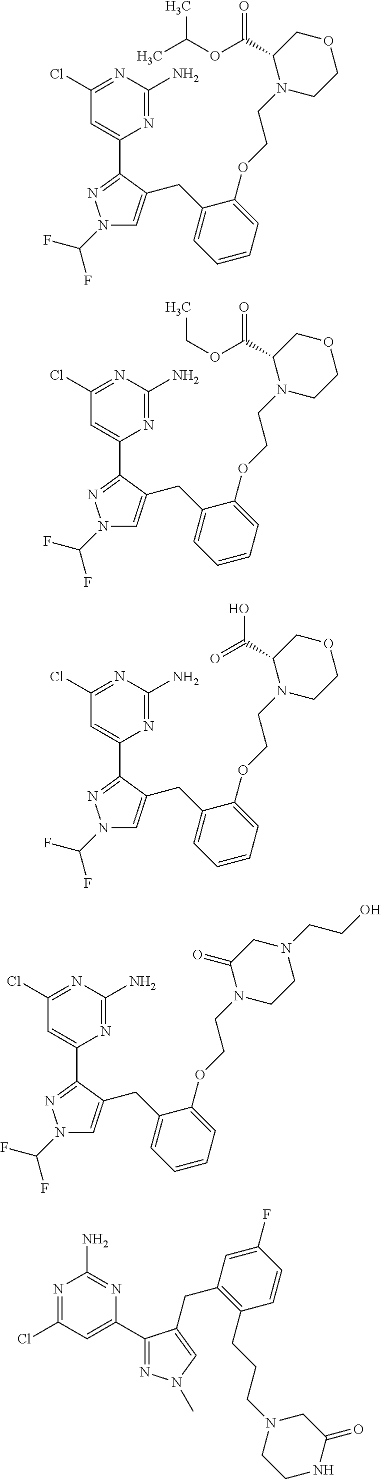 [SMILES: CC(C)OC(=O)[C@@H]1COCCN1CCOc1ccccc1Cc1cn(C(F)F)nc1-c1cc(Cl)nc(N)n1 ; CCOC(=O)[C@@H]1COCCN1CCOc1ccccc1Cc1cn(C(F)F)nc1-c1cc(Cl)nc(N)n1 ; Nc1nc(Cl)cc(-c2nn(C(F)F)cc2Cc2ccccc2OCCN2CCOC[C@H]2C(=O)O)n1 ; Nc1nc(Cl)cc(-c2nn(C(F)F)cc2Cc2ccccc2OCCN2CCN(CCO)CC2=O)n1 ; Cn1cc(Cc2cc(F)ccc2CCCN2CCNC(=O)C2)c(-c2cc(Cl)nc(N)n2)n1]

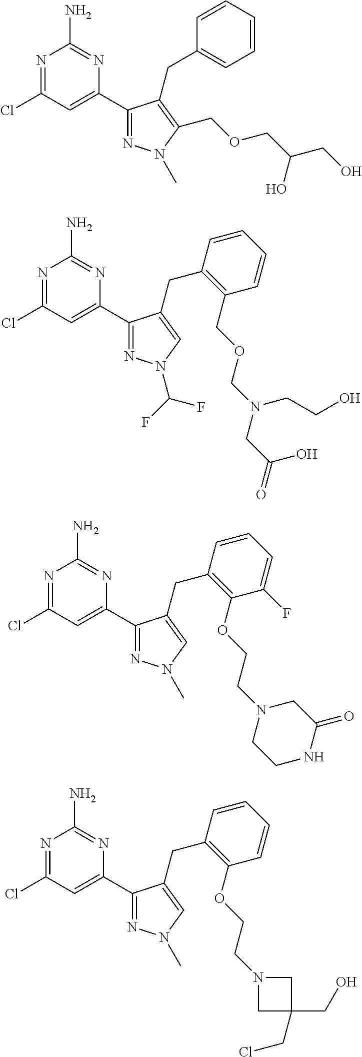

and pharmaceutically acceptable salts, hydrates, solvates, polymorphs, co-crystals, tautomers, stereoisomers, isotopically labeled derivatives, and prodrugs thereof.

References to compounds provided herein, including references to compounds of Formula (I), are intended to include compounds of all generic and subgeneric formulae recited herein (e.g., Formulae (I), (II), (III), (IV), (V), (VI), (VII), and subgeneric formulae thereof), as well as all specific compounds recited herein.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

The following chemical group definitions and embodiments apply to all generic and subgeneric formulae recited herein (e.g., Formulae (I), (II), (III), (IV), (V), (VI), (VII), and subgeneric formulae thereof).

G, R¹, and R^{N1}

As defined herein, G is halogen, —CN, optionally substituted alkyl, or optionally substituted acyl. In certain embodiments, G is halogen. In certain embodiments, G is optionally substituted alkyl. In certain embodiments, G is —CN. In certain embodiments, G is optionally substituted acyl.

In certain embodiments, G is —Br. In certain embodiments, G is —I. In certain embodiments, G is —F. In certain embodiments, G is —Cl.

In certain embodiments, G is C₁₋₆ haloalkyl. In certain embodiments, G is C₁₋₃₆ haloalkyl. In certain embodiments, G is halomethyl. In certain embodiments, G is trihalomethyl. In certain embodiments, G is —CF₃.

As defined herein, R¹ is hydrogen, halogen, optionally substituted alkyl, or optionally substituted acyl. In certain embodiments, R¹ is hydrogen. In certain embodiments, R¹ is halogen. In certain embodiments, R¹ is optionally substituted alkyl. In certain embodiments, R¹ is optionally substituted acyl.

In certain embodiments, R¹ is optionally substituted C₁₋₆ alkyl. In certain embodiments, R¹ is unsubstituted C₁₋₆ alkyl. In certain embodiments, R¹ is optionally substituted C₁₋₃ alkyl. In certain embodiments, R¹ is unsubstituted C₁₋₃ alkyl. In certain embodiments, R¹ is selected from the group consisting of methyl, ethyl, n-propyl, iso-propyl, n-butyl, iso-butyl, sec-butyl, and tert-butyl. In certain embodiments, R¹ is methyl.

As defined herein, each instance of R^{N1} is independently hydrogen, optionally substituted alkyl, optionally substituted acyl, or a nitrogen protecting group, or optionally two R^{N1} are taken together with the intervening atoms to form optionally substituted heterocyclyl or optionally substituted heteroaryl. In certain embodiments, at least one instance of R^{N1} is hydrogen. In certain embodiments, at least one instance of R^{N1} is optionally substituted alkyl. In certain embodiments, at least one instance of R^{N1} is optionally substituted acyl. In certain embodiments, at least one instance of R^{N1} is a nitrogen protecting group. In certain embodiments, two R^{N1} are taken together with the intervening atoms to form optionally substituted heterocyclyl. In certain embodiments, two R^{N1} are taken together with the intervening atoms to form optionally substituted heteroaryl.

In certain embodiments, at least one instance of R^{N1} is optionally substituted C₁₋₆ alkyl. In certain embodiments, at least one instance of R^{N1} is unsubstituted C₁₋₆ alkyl. In certain embodiments, at least one instance of R^{N1} is optionally substituted C₁₋₃ alkyl. In certain embodiments, at least one instance of R^{N1} is unsubstituted C₁₋₃ alkyl. In certain embodiments, at least one instance of R^{N1} is selected from the group consisting of methyl, ethyl, n-propyl, iso-propyl, n-butyl, iso-butyl, sec-butyl, and tert-butyl.

In certain embodiments, both instances of R^{N1} are hydrogen.

In certain embodiments, G is —Cl; and R¹ is hydrogen. In certain embodiments, G is —Cl; and both instances of R^{N1} are hydrogen. In certain embodiments, R¹ is hydrogen; and both instances of R^{N1} are hydrogen. In certain embodiments, G is —Cl; R¹ is hydrogen; and both instances of R^{N1} are hydrogen.

### Ring A, Y, and R³

As defined herein, A (also “Ring A”) is an optionally substituted monocyclic heteroaryl ring comprising at least 1 nitrogen atom. In certain embodiments, A is an optionally substituted 5-membered heteroaryl ring comprising 1, 2, or 3 nitrogen atoms. In certain embodiments, A is an optionally substituted 5-membered heteroaryl ring comprising 2 or 3 nitrogen atoms.

In certain embodiments, A is an optionally substituted 5-membered heteroaryl ring comprising 2 nitrogen atoms. In certain embodiments, Ring A is an optionally substituted pyrazole ring. In certain embodiments, Ring A is an optionally substituted imidazole ring.

In certain embodiments, A is an optionally substituted 5-membered heteroaryl ring comprising 3 nitrogen atoms. In certain embodiments, Ring A is an optionally substituted triazole ring. In certain embodiments, Ring A is an optionally substituted 1,2,3-triazole ring. In certain embodiments, Ring A is an optionally substituted 1,2,4-triazole ring.

In certain embodiments, the group -(A)-Y—R³ is of the formula:

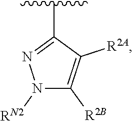

wherein one of R^{2A} and R^{2B} is —Y—R³. In certain embodiments, -(A)-Y—R³ is of the formula:

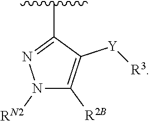

As defined herein, Y is a bond, optionally substituted alkylene, optionally substituted heteroalkylene, —O—, —NR^{N}—, —S—, —S(═O)—, or —SO₂—. In certain embodiments, Y is a bond. In certain embodiments, Y is optionally substituted alkylene. In certain embodiments, Y is optionally substituted heteroalkylene. In certain embodiments, Y is —O—. In certain embodiments, Y is —NR^{N}—. In certain embodiments, Y is —S—. In certain embodiments, Y is —S(═O)—. In certain embodiments, Y is —SO₂—.

In certain embodiments, Y is optionally substituted C₁₋₆ alkylene. In certain embodiments, Y is unsubstituted C₁₋₆ alkylene. In certain embodiments, Y is optionally substituted C₁₋₃ alkylene. In certain embodiments, Y is unsubstituted C₁₋₃ alkylene. In certain embodiments, Y is optionally substituted methylene. In certain embodiments, Y is unsubstituted methylene.

As defined herein, R³ is optionally substituted carbocyclyl, optionally substituted heterocyclyl, optionally substituted aryl, or optionally substituted heteroaryl. In certain embodiments, R³ is optionally substituted carbocyclyl. In certain embodiments, R³ is optionally substituted heterocyclyl. In certain embodiments, R³ is optionally substituted aryl. In certain embodiments, R³ is or optionally substituted heteroaryl.

In certain embodiments, R³ is optionally substituted thiophenyl. In certain embodiments, R³ is unsubstituted thiophenyl.

In certain embodiments, R³ is optionally substituted C₃₋₆ carbocyclyl. In certain embodiments, R³ is unsubstituted C₃₋₆ carbocyclyl. In certain embodiments, R³ is optionally substituted cyclobutyl. In certain embodiments, R³ is unsubstituted cyclobutyl.

In certain embodiments, R³ is optionally substituted C₆₋₁₄ aryl. In certain embodiments, R³ is optionally substituted phenyl. In certain embodiments, R³ is unsubstituted phenyl. In certain embodiments, R³ is of the formula:

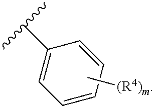

In certain embodiments, R³ is of the formula:

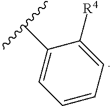

In certain embodiments, R³ is of the formula:

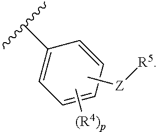

In certain embodiments, R³ is of the formula:

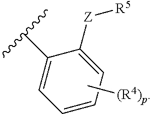

In certain embodiments, R³ is of the formula:

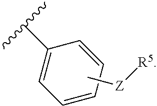

In certain embodiments, R³ is of the formula:

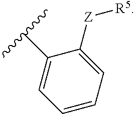

R^{2A}, R^{2B}, and R^{N2}

As defined herein, R^{2A} independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, —SR^{S}, or —Y—R³. In certain embodiments, R^{2A} is hydrogen. In certain embodiments, R^{2A} is halogen. In certain embodiments, R^{2A} is —CN. In certain embodiments, R^{2A} is —N₃. In certain embodiments, R^{2A} is —NO₂. In certain embodiments, R^{2A} is optionally substituted alkyl. In certain embodiments, R^{2A} is optionally substituted alkenyl. In certain embodiments, R^{2A} is optionally substituted alkynyl. In certain embodiments, R^{2A} is optionally substituted aryl. In certain embodiments, R^{2A} is optionally substituted heteroaryl. In certain embodiments, R^{2A} is optionally substituted carbocyclyl. In certain embodiments, R^{2A} is optionally substituted heterocyclyl. In certain embodiments, R^{2A} is optionally substituted acyl. In certain embodiments, R^{2A} is —OR^{O}. In certain embodiments, R^{2A} is —N(R^{N})₂. In certain embodiments, R^{2A} is —SR^{S}. In certain embodiments, R^{2A} is —Y—R³.

As described herein, one of R^{2A} and R^{2B} is —Y—R³. In certain embodiments, one and only one of R^{2A} and R^{2B} is —Y—R³. In certain embodiments, R^{2A} is —Y—R³; and R^{2B} is hydrogen. In certain embodiments, R^{2A} is —Y—R³; and R^{2B} is methyl.

In certain embodiments, R^{2A} is of the formula:

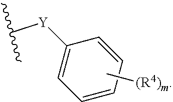

In certain embodiments, R^{2A} is of the formula:

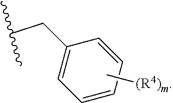

In certain embodiments, R^{2A} is of the formula:

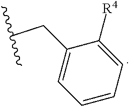

In certain embodiments, R^{2A} is of the formula:

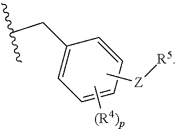

In certain embodiments, R^{2A} is of the formula:

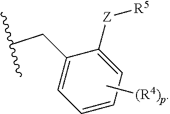

In certain embodiments, R^{2A} is of the formula:

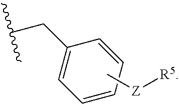

In certain embodiments, R^{2A} is of the formula:

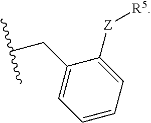

As defined herein, R^{2B} independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, —SR^{S}, or —Y—R³. In certain embodiments, R^{2B} is hydrogen. In certain embodiments, R^{2B} is halogen. In certain embodiments, R^{2B} is —CN. In certain embodiments, R^{2B} is —N₃. In certain embodiments, R^{2B} is —NO₂. In certain embodiments, R^{2B} is optionally substituted alkyl. In certain embodiments, R^{2B} is optionally substituted alkenyl. In certain embodiments, R^{2B} is optionally substituted alkynyl. In certain embodiments, R^{2B} is optionally substituted aryl. In certain embodiments, R^{2B} is optionally substituted heteroaryl. In certain embodiments, R^{2B} is optionally substituted carbocyclyl. In certain embodiments, R^{2B} is optionally substituted heterocyclyl. In certain embodiments, R^{2B} is optionally substituted acyl. In certain embodiments, R^{2B} is —OR^{O}. In certain embodiments, R^{2B} is —N(R^{N})₂. In certain embodiments, R^{2B} is —SR^{S}. In certain embodiments, R^{2B} is —Y—R³.

In certain embodiments, R^{2B} is optionally substituted C₁₋₆ alkyl. In certain embodiments, R^{2B} is unsubstituted C₁₋₆ alkyl. In certain embodiments, R^{2B} is optionally substituted C₁₋₃ alkyl. In certain embodiments, R^{2B} is unsubstituted C₁₋₃ alkyl. In certain embodiments, R^{2B} is selected from the group consisting of methyl, ethyl, n-propyl, iso-propyl, n-butyl, iso-butyl, sec-butyl, and tert-butyl. In certain embodiments, R^{2B} is methyl.

In certain embodiments, R^{2B} is optionally substituted C₁₋₆ acyl. In certain embodiments, R^{2B} is unsubstituted C₁₋₆ acyl. In certain embodiments, R^{2B} is optionally substituted C₁₋₃ acyl. In certain embodiments, R^{2B} is unsubstituted C₁₋₃ acyl.

In certain embodiments, R^{2B} is —CH₂OH, —CH₂OCH₂Ph, —CH₂O(C═O)Ph, or —CH₂CO₂Me.

In certain embodiments, R^{2B} is —CO₂H, —CO₂Me, or —CO₂CH₂Ph.

In certain embodiments, R^{2B} is of one of the following formulae:

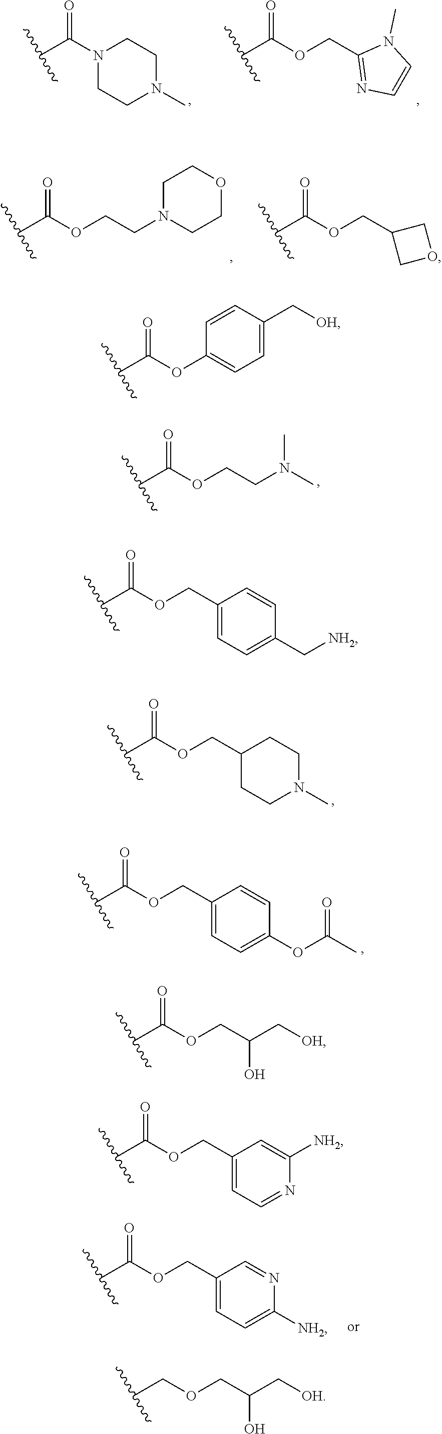

As defined herein, R^{N2} is hydrogen, optionally substituted alkyl, optionally substituted acyl, or a nitrogen protecting group. In certain embodiments, R^{N2} is hydrogen. In certain embodiments, R^{N2} is optionally substituted alkyl. In certain embodiments, R^{N2} is optionally substituted acyl. In certain embodiments, R^{N2} is a nitrogen protecting group.

In certain embodiments, R^{N2} is optionally substituted C₁₋₆ alkyl. In certain embodiments, R^{N2} is unsubstituted C₁₋₆ alkyl. In certain embodiments, R^{N2} is optionally substituted C₁₋₃ alkyl. In certain embodiments, R^{N2} is unsubstituted C₁₋₁₃ alkyl. In certain embodiments, R^{N2} is selected from the group consisting of methyl, ethyl, n-propyl, iso-propyl, n-butyl, iso-butyl, sec-butyl, and tert-butyl. In certain embodiments, R^{N2} is methyl. In certain embodiments, R^{N2} is ethyl. In certain embodiments, R^{N2} is —C(²H)₃.

In certain embodiments, R^{N2} is haloalkyl. In certain embodiments, R^{N2} is C₁₋₆ haloalkyl. In certain embodiments, R^{N2} is C₁₋₃ haloalkyl. In certain embodiments, R^{N2} is dihalomethyl. In certain embodiments, R^{N2} is trihalomethyl. In certain embodiments, R^{N2} is —CHF₂. In certain embodiments, R^{N2} is —CH₂F. In certain embodiments, R^{N2} is —CF₃.

In certain embodiments, R^{2A} is —Y—R³; R^{2B} is hydrogen; and R^{2N} is hydrogen, methyl, or —CHF₂. In certain embodiments, R^{2A} is —Y—R³; R^{2B} is methyl; and R^{2N} is hydrogen, methyl, or —CHF₂.

R⁴, Z, R⁵, m, and p

As defined herein, each instance of R⁴ is independently halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, or —SR^{S}. In certain embodiments, at least one instance of R⁴ is halogen. In certain embodiments, at least one instance of R⁴ is —CN. In certain embodiments, at least one instance of R⁴ is —N₃. In certain embodiments, at least one instance of R⁴ is —NO₂. In certain embodiments, at least one instance of R⁴ is optionally substituted alkyl. In certain embodiments, at least one instance of R⁴ is optionally substituted alkenyl. In certain embodiments, at least one instance of R⁴ is optionally substituted alkynyl. In certain embodiments, at least one instance of R⁴ is optionally substituted aryl. In certain embodiments, at least one instance of R⁴ is optionally substituted heteroaryl. In certain embodiments, at least one instance of R⁴ is optionally substituted carbocyclyl. In certain embodiments, at least one instance of R⁴ is optionally substituted heterocyclyl. In certain embodiments, at least one instance of R⁴ is optionally substituted acyl. In certain embodiments, at least one instance of R⁴ is —OR^{O}. In certain embodiments, at least one instance of R⁴ is —N(R^{N})₂. In certain embodiments, at least one instance of R⁴ is —SR^{S}.

In certain embodiments, at least one instance of R⁴ is halogen. In certain embodiments, at least one instance of R⁴ is —Cl. In certain embodiments, at least one instance of R⁴ is —F. In certain embodiments, at least one instance of R⁴ is —I. In certain embodiments, at least one instance of R⁴ is —Br.

In certain embodiments, at least one instance of R⁴ is optionally substituted C₁₋₆ alkyl. In certain embodiments, at least one instance of R⁴ is unsubstituted C₁₋₆ alkyl. In certain embodiments, at least one instance of R⁴ is optionally substituted C₁₋₃ alkyl. In certain embodiments, at least one instance of R⁴ is unsubstituted C₁₋₃ alkyl. In certain embodiments, at least one instance of R⁴ is selected from the group consisting of methyl, ethyl, n-propyl, iso-propyl, n-butyl, iso-butyl, sec-butyl, and tert-butyl.

In certain embodiments, at least one instance of R⁴ is optionally substituted C₁₋₆ acyl. In certain embodiments, at least one instance of R⁴ is unsubstituted C₁₋₆ acyl. In certain embodiments, at least one instance of R⁴ is optionally substituted C₁₋₃ acyl. In certain embodiments, at least one instance of R⁴ is unsubstituted C₁₋₃ acyl.

In certain embodiments, at least one instance of R⁴ is —CO₂H, —CO₂Me, —CO₂CH₂Ph, —CH₂OCH₂CH₂NMe₂, —C(═O)NHCH₂Ph, —C(═O)NHMe, —C(═O)NHCH₂CH₂OMe, or —CO₂CH₂CH₂CH₂NMe₂. In certain embodiments, at least one instance of R⁴ is of the formula:

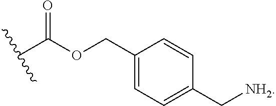 [SMILES: *C(=O)OCc1ccc(CN)cc1]

In certain embodiments, at least one instance of R⁴ is optionally substituted C₃₋₆ carbocyclyl. In certain embodiments, at least one instance of R⁴ is unsubstituted C₃₋₆ carbocyclyl. In certain embodiments, at least one instance of R⁴ is optionally substituted cyclopropyl. In certain embodiments, at least one instance of R⁴ is of the formula:

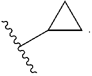 [SMILES: *C1CC1]

In certain embodiments, at least one instance of R is optionally substituted C₆₋₁₄ aryl. In certain embodiments, at least one instance of R⁴ is unsubstituted C₆₋₁₄ aryl. In certain embodiments, at least one instance of R⁴ is optionally substituted phenyl. In certain embodiments, at least one instance of R⁴ is unsubstituted phenyl.

In certain embodiments, at least one instance of R⁴ is —OR^{O}. In certain embodiments, at least one instance of R⁴ is —OMe, —OCF₃, —OCH₂CO₂Me, or —O(CH₂CH₂O)₃Me.

In certain embodiments, at least one instance of R⁴ is of one of the following formulae:

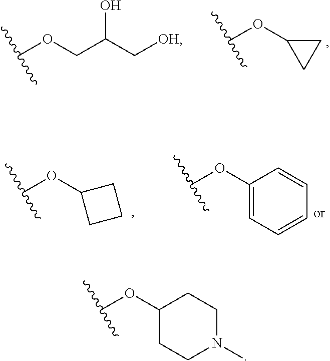

In certain embodiments, at least one instance of R⁴ is —Z—R⁵. In certain embodiments, only one instance of R⁴ is —Z—R⁵.

As defined herein, Z is a bond, optionally substituted alkylene, optionally substituted heteroalkylene, or optionally substituted acylene. In certain embodiments, Z is a bond. In certain embodiments, Z is optionally substituted alkylene. In certain embodiments, Z is optionally substituted heteroalkylene. In certain embodiments, Z is optionally substituted acylene.

In certain embodiments, Z is optionally substituted C₁₋₆ alkylene. In certain embodiments, Z is unsubstituted C₁₋₆ alkylene. In certain embodiments, Z is optionally substituted C₁₋₃ alkylene. In certain embodiments, Z is unsubstituted C₁₋₃ alkylene.

In certain embodiments, Z is optionally substituted C₁₋₆ acylene. In certain embodiments, Z is unsubstituted C₁₋₆ acylene. In certain embodiments, Z is optionally substituted C₁₋₃ acylene. In certain embodiments, Z is unsubstituted C₁₋₃ acylene.

In certain embodiments, Z is optionally substituted C₁₋₆heteroalkylene. In certain embodiments, Z is unsubstituted C₁₋₆ heteroalkylene. In certain embodiments, Z is optionally substituted C₁₋₃ heteroalkylene. In certain embodiments, Z is unsubstituted C₁₋₃ heteroalkylene.

In certain embodiments, Z is optionally substituted C₁₋₆ heteroalkylene comprising 1-3 heteroatoms independently selected from O, N, and S. In certain embodiments, Z is unsubstituted C₁₋₆ heteroalkylene comprising 1-3 heteroatoms independently selected from O, N, and S. In certain embodiments, Z is optionally substituted C₁₋₃ heteroalkylene comprising 1-3 heteroatoms independently selected from O, N, and S. In certain embodiments, Z is unsubstituted C₁₋₃ heteroalkylene comprising 1-3 heteroatoms independently selected from O, N, and S.

In certain embodiments, Z is optionally substituted C₁₋₆ heteroalkylene comprising 1 or 2 heteroatoms independently selected from O and N. In certain embodiments, Z is unsubstituted C₁₋₆ heteroalkylene comprising 1 or 2 heteroatoms independently selected from O and N. In certain embodiments, Z is optionally substituted C₁₋₃ heteroalkylene comprising 1 or 2 heteroatoms independently selected from O and N. In certain embodiments, Z is unsubstituted C₁₋₃ heteroalkylene comprising 1 or 2 heteroatoms independently selected from O and N.

In certain embodiments, Z is of one of the following formulae:

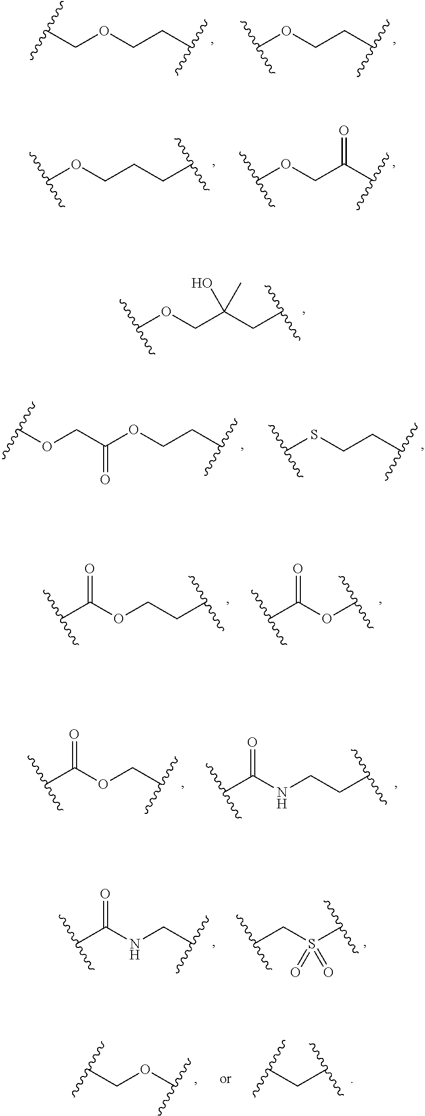

In certain embodiments, Z is of the formula:

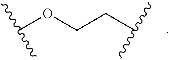 [SMILES: *CCO* ; "."]

As defined herein, R⁵ is optionally substituted heterocyclyl, optionally substituted heteroaryl, —N(R^{N})₂, or —OR^{O}. In certain embodiments, R⁵ is optionally substituted heterocyclyl. In certain embodiments, R⁵ is optionally substituted heteroaryl. In certain embodiments, R⁵ is —N(R^{N})₂. In certain embodiments, R⁵ is —OR^{O}.

In certain embodiments, R⁵ is optionally substituted 4- to 7-membered heterocyclyl. In certain embodiments, R⁵ is optionally substituted 4- to 7-membered heterocyclyl comprising 1, 2, or 3 heteroatoms independently selected from N and O. In certain embodiments, R⁵ is unsubstituted 4- to 7-membered heterocyclyl comprising 1, 2, or 3 heteroatoms independently selected from N and O. In certain embodiments, R⁵ is optionally substituted 5- or 6-membered heterocyclyl comprising 1 or 2 heteroatoms independently selected from N and O. In certain embodiments, R⁵ is unsubstituted 5- or 6-membered heterocyclyl comprising 1 or 2 heteroatoms independently selected from N and O. In certain embodiments, R⁵ is optionally substituted 5-membered heterocyclyl comprising 1 or 2 heteroatoms independently selected from N and O. In certain embodiments, R⁵ is unsubstituted 5-membered heterocyclyl comprising 1 or 2 heteroatoms independently selected from N and O. In certain embodiments, R⁵ is optionally substituted 6-membered heterocyclyl comprising 1 or 2 heteroatoms independently selected from N and O. In certain embodiments, R⁵ is unsubstituted 6-membered heterocyclyl comprising 1 or 2 heteroatoms independently selected from N and O.

In certain embodiments, R⁵ is optionally substituted morpholinyl. In certain embodiments, R⁵ is unsubstituted morpholinyl. In certain embodiments, R⁵ is optionally substituted piperidinyl. In certain embodiments, R⁵ is unsubstituted piperidinyl. In certain embodiments, R⁵ is optionally substituted piperazinyl. In certain embodiments, R⁵ is unsubstituted piperazinyl. In certain embodiments, R⁵ is optionally substituted pyrrolidinyl. In certain embodiments, R⁵ is unsubstituted pyrrolidinyl.

In certain embodiments, R⁵ is of one of the following formulae:

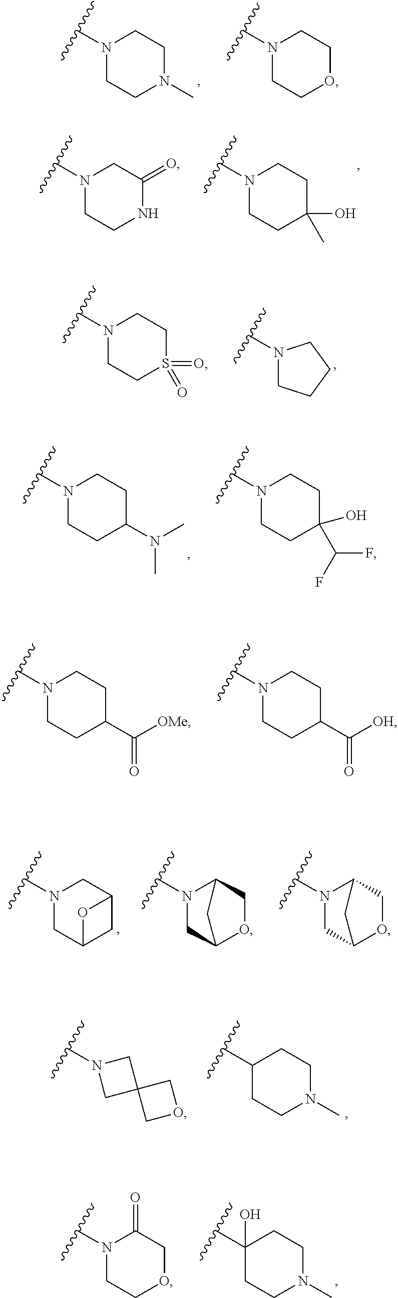

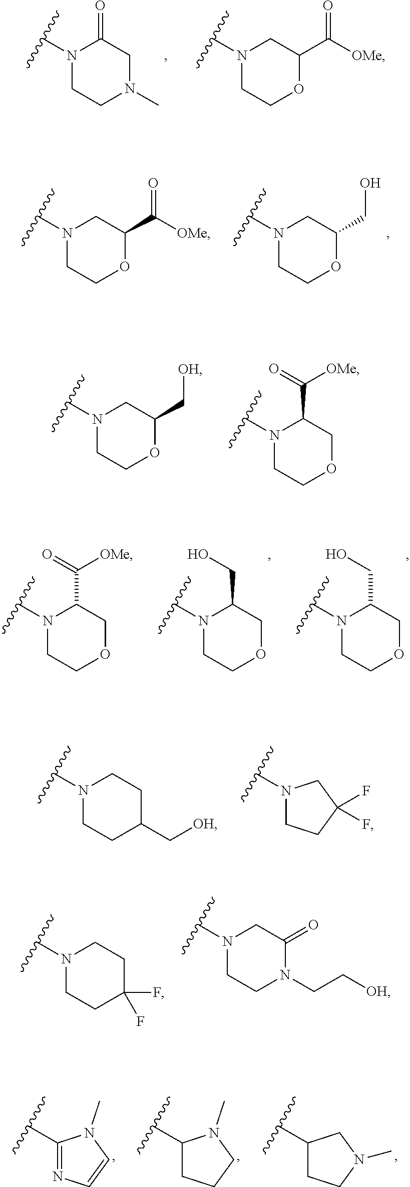

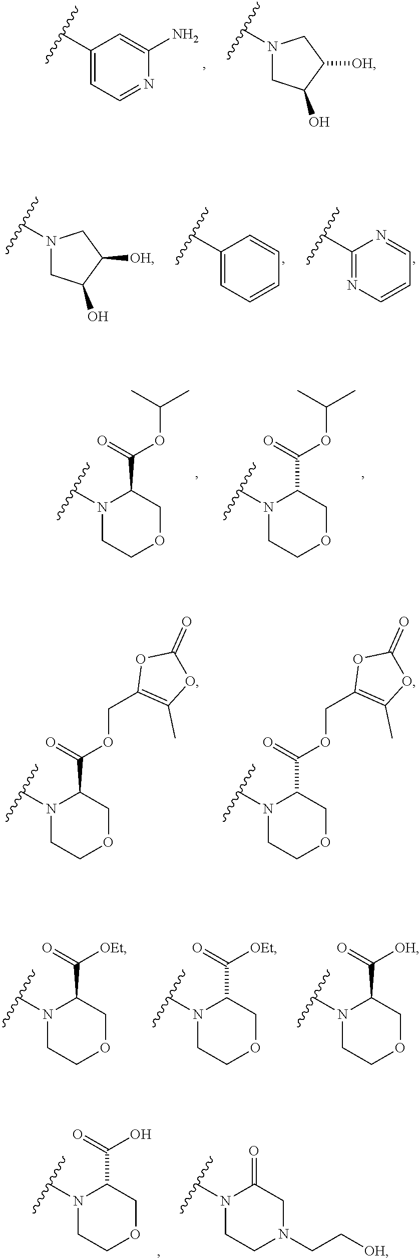

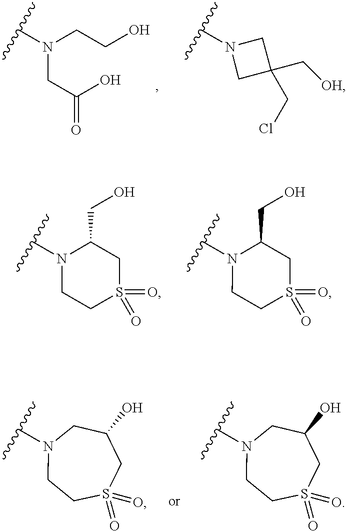

In certain embodiments, at least one instance of R⁴ is of one of the following formulae:

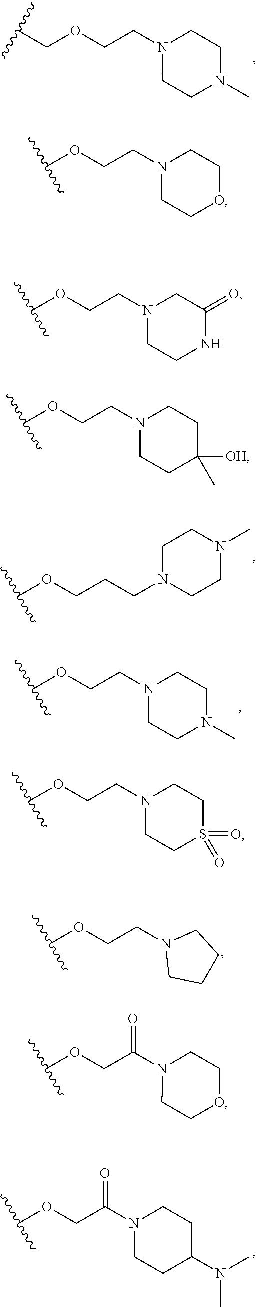

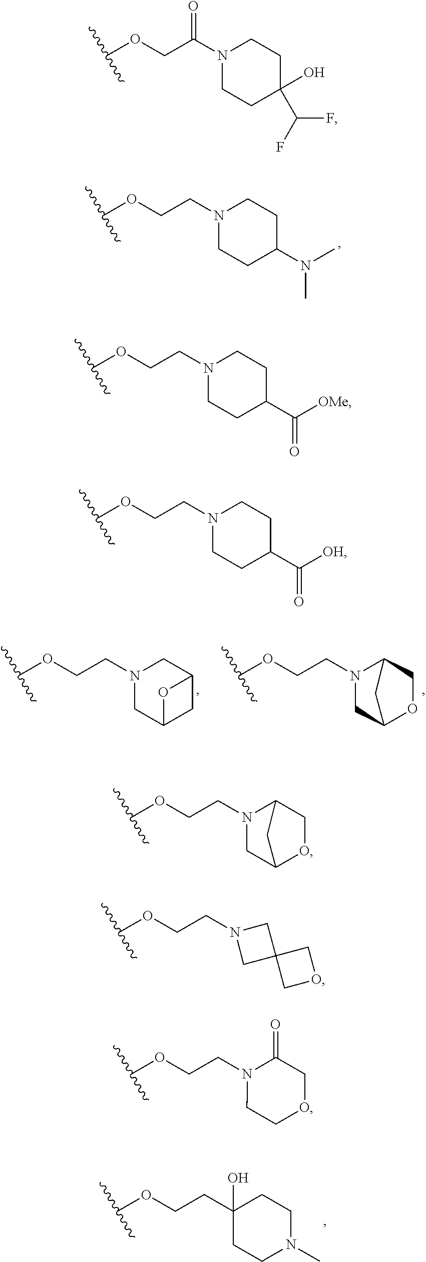

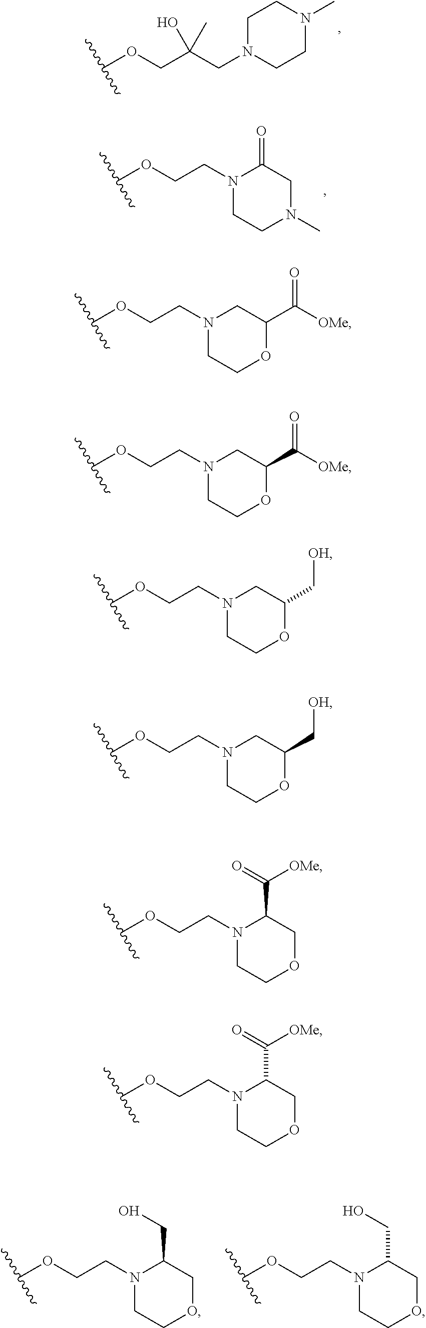

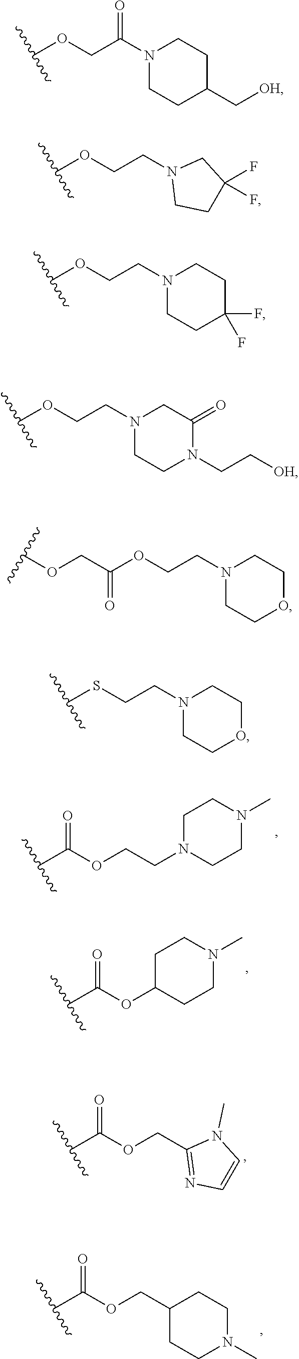

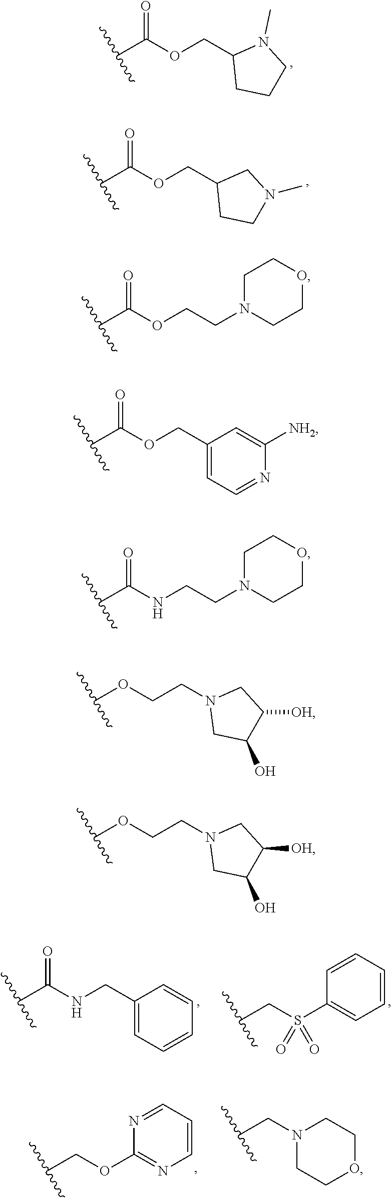

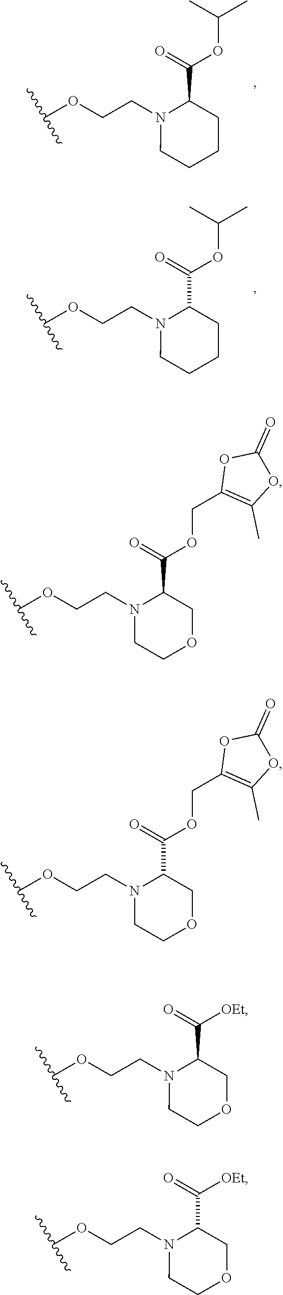

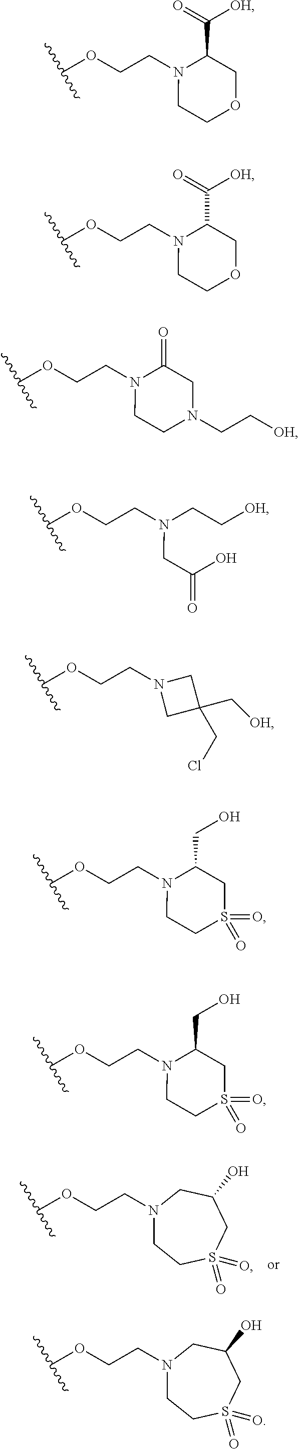

In certain embodiments, at least one instance of —Z—R⁵ is of one of the foregoing formulae. In certain embodiments, one instance of —Z—R⁵ is of one of the foregoing formulae.

As defined herein, m is 0, 1, 2, 3, 4, or 5. In certain embodiments, m is 0. In certain embodiments, m is 1. In certain embodiments, m is 2. In certain embodiments, m is 3. In certain embodiments, m is 4. In certain embodiments, m is 5.

As defined herein, p is 0, 1, 2, 3, or 4. In certain embodiments, pm is 0. In certain embodiments, p is 1. In certain embodiments, p is 2. In certain embodiments, p is 3. In certain embodiments, p is 4.

R^{N}, R^{O}, and R^{S}

As defined herein, each instance of R^{N} is independently hydrogen, optionally substituted alkyl, optionally substituted acyl, or a nitrogen protecting group, or optionally two R^{N} are taken together with the intervening atoms to form optionally substituted heterocyclyl or optionally substituted heteroaryl. In certain embodiments, at least one instance of R^{N} is hydrogen. In certain embodiments, at least one instance of R^{N} is optionally substituted alkyl. In certain embodiments, at least one instance of R^{N} is optionally substituted acyl. In certain embodiments, at least one instance of R^{N} is a nitrogen protecting group. In certain embodiments, two R^{N} are taken together with the intervening atoms to form optionally substituted heterocyclyl. In certain embodiments, two R^{N} are taken together with the intervening atoms to form optionally substituted heteroaryl.

In certain embodiments, at least one instance of R^{N} is optionally substituted C₁₋₆ alkyl. In certain embodiments, at least one instance of R^{N} is unsubstituted C₁₋₆ alkyl. In certain embodiments, at least one instance of R^{N} is optionally substituted C₁₋₃ alkyl. In certain embodiments, at least one instance of R^{N} is unsubstituted C₁₋₃ alkyl. In certain embodiments, at least one instance of R^{N} is selected from the group consisting of methyl, ethyl, n-propyl, iso-propyl, n-butyl, iso-butyl, sec-butyl, and tert-butyl.

As defined herein, each instance of R^{O} is independently hydrogen, optionally substituted alkyl, optionally substituted acyl, or an oxygen protecting group. In certain embodiments, at least one instance of R^{O} is hydrogen. In certain embodiments, at least one instance of R^{O} is optionally substituted alkyl. In certain embodiments, at least one instance of R^{O} is optionally substituted acyl. In certain embodiments, at least one instance of R^{O} is an oxygen protecting group.

In certain embodiments, at least one instance of R^{O} is optionally substituted C₁₋₆ alkyl. In certain embodiments, at least one instance of R^{O} is unsubstituted C₁₋₆ alkyl. In certain embodiments, at least one instance of R^{O} is optionally substituted C₁₋₃ alkyl. In certain embodiments, at least one instance of R^{O} is unsubstituted C₁₋₃ alkyl. In certain embodiments, at least one instance of R^{O} is selected from the group consisting of methyl, ethyl, n-propyl, iso-propyl, n-butyl, iso-butyl, sec-butyl, and tert-butyl.

As defined herein, each instance of R^{S} is independently hydrogen, optionally substituted alkyl, optionally substituted acyl, or a sulfur protecting group. As defined herein, each instance of R^{S} is independently hydrogen, optionally substituted alkyl, optionally substituted acyl, or an oxygen protecting group. In certain embodiments, at least one instance of R^{S} is hydrogen. In certain embodiments, at least one instance of R^{S} is optionally substituted alkyl. In certain embodiments, at least one instance of R^{S} is optionally substituted acyl. In certain embodiments, at least one instance of R^{S} is a sulfur protecting group.

In certain embodiments, at least one instance of R^{S} is optionally substituted C₁₋₆ alkyl. In certain embodiments, at least one instance of R^{S} is unsubstituted C₁₋₆ alkyl. In certain embodiments, at least one instance of R^{S} is optionally substituted C₁₋₃ alkyl. In certain embodiments, at least one instance of R^{S} is unsubstituted C₁₋₃ alkyl. In certain embodiments, at least one instance of R^{S} is selected from the group consisting of methyl, ethyl, n-propyl, iso-propyl, n-butyl, iso-butyl, sec-butyl, and tert-butyl.

### Pharmaceutical Compositions, Kits, and Administration

The present disclosure provides pharmaceutical compositions comprising a compound described herein (e.g., a compound of Formula (I), or a pharmaceutically acceptable salt, solvate, hydrate, polymorph, co-crystal, tautomer, stereoisomer, isotopically labeled derivative, or prodrug thereof), and a pharmaceutically acceptable carrier or excipient. In certain embodiments, the compound described herein is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents, such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, foams, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. A formulation suitable for topical administration may be in the form of a gel or foam. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient. In certain embodiments, the compound or composition is administered via intravaginal ring or film (e.g., to provide slow (i.e., extended) release of a compound or composition described herein). In certain embodiments, the intravaginal ring or film delivers a compound or composition provided herein over the course of hours, days, weeks, or months to the subject. In certain embodiments, the compound or composition is administered intravaginally in the form of a gel or foam. In certain embodiments, the compound or composition is administered intravaginally in the form of a lubricant (e.g., a personal lubricant suitable for use in intercourse).

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds and compositions provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), ocular, mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound or composition required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compounds and the additional pharmaceutical agent, but not both.

The compound or pharmaceutical composition thereof can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

In certain embodiments, the additional pharmaceutical agent is a pharmaceutical agent useful for treating and/or preventing a disease or condition. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents.

Also encompassed by the disclosure are kits (e.g., pharmaceutical packs). The kits provided may comprise a compound or pharmaceutical composition described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease or condition in a subject in need thereof. In certain embodiments, the kits are useful for preventing a disease or condition in a subject. In certain embodiments, the kits are useful for contraception.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. A kit described herein may include one or more additional pharmaceutical agents described herein as a separate composition. In certain embodiments, the kits useful for contraception further comprise a means for reminding the subject to take the compound or composition at regular intervals.

In certain embodiments, a kit described herein is kit for use in contraception (e.g., male or female contraception). In certain embodiments, the kit comprises a compound or composition described herein in oral dosage form. In certain embodiments, the kit comprises a compound or composition described herein in an intravaginal ring or film (e.g., to provide slow (i.e., extended) release of a compound or composition described herein). In certain embodiments, the kit comprises a compound or composition described herein in the form of a gel or foam for topical and/or intravaginal administration. In certain embodiments, the kit comprises a compound or composition described herein in the form of a lubricant (e.g., a personal lubricant suitable for use in intercourse). In certain embodiments, the kit comprises instructions for use, e.g., instructions to use the compound or composition prior to and/or during intercourse. In certain embodiments, the kit comprises a means for reminding the subject to take the compound or composition at regular intervals.

### Methods of Treatment and Uses

Provided herein are methods of treating and/or preventing a disease or condition in a subject, the methods comprising administering to the subject a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use in treating and/or preventing a disease or condition in a subject. Also provided herein are uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of medicaments for treating and/or preventing diseases or conditions in a subject. In certain embodiments, the disease or condition is typically associated with the activity of a sAC enzyme.

In certain embodiments, the disease or condition to be treated or prevented is a proliferative disease (e.g., cancer, a disease associated with angiogenesis, a neoplasm), inflammatory disease, autoimmune disease, painful condition, infectious disease, liver disease, pulmonary disease, neurological disease, musculoskeletal disease, metabolic disorder (e.g., a diabetic condition), or an ocular condition.

In certain embodiments, the disease or condition is associated with the activity of a sAC enzyme in a subject. In certain embodiments, the disease or condition is associated with aberrant activity (e.g., increased activity) of a sAC enzyme in a subject. In certain embodiments, the disease or condition is associated with increased activity of a sAC enzyme in a subject. In certain embodiments, the disease or condition is associated with normal or baseline level activity of a sAC enzyme in a subject.

In certain embodiments, a sAC inhibitor described herein is used to treat cancer, to inhibit insulin secretion, elevate intraocular pressure, or as a contraceptive agent, e.g., as described in International Application Publication No. WO 2001/085753; the entire contents of which is incorporated herein by reference. In certain embodiments, a sAC inhibitor described herein is used to treat cancer. In certain embodiments, a sAC inhibitor described herein is used for inhibiting insulin secretion. In certain embodiments, a sAC inhibitor described herein is used to elevate intraocular pressure (IOP). In certain embodiments, a sAC inhibitor described herein is used as a contraceptive agent.

In certain embodiments, a sAC inhibitor described herein is used as anti-inflammatory agent, e.g., as described in International Application Publication No. WO 2006/113236; the entire contents of which is incorporated herein by reference.

In certain embodiments, a sAC inhibitor described herein is used to treat an infectious disease (e.g., a bacterial infection), e.g., as described in International Application Publication No. WO 2008/121171; and International Application Publication No. WO 2008/088771; the entire contents of each of which is incorporated herein by reference.

In certain embodiments, a sAC inhibitor described herein is used to treat proliferative diseases (e.g., cancer, e.g., prostate cancer), e.g., as described in International Application Publication No. WO 2014/093460; the entire contents of which is incorporated herein by reference.

In certain embodiments, a sAC inhibitor described herein is used to increase melanin production for disease treatment or as a tanning/hair darkening agent, e.g., as described in International Application Publication No. WO 2018/006039; the entire contents of which is incorporated by reference. In certain embodiments, a sAC inhibitor described herein is used to increase melanin production. In certain embodiments, a sAC inhibitor described herein is used as a tanning/hair darkening agent. In certain embodiments, a sAC inhibitor described herein can be used to prevent cancer in the skin. In certain embodiments, a sAC inhibitor described herein can be used to prevent sun-induced diseases, such as porphyria. In certain embodiments, a sAC inhibitor described herein can be used as an anti-aging treatment. Without wishing to be bound by a particular theory, a sAC inhibitor described herein can be used to increase melanin levels in the skin and can therefore be used to treat and/or prevent a variety of skin disorders.

For a review of sAC biology and uses for sAC inhibitors, see Wiggins et al. “Pharmacological modulation of the CO₂/HCO₃⁻/pH-, calcium-, and ATP-sensing soluble adenylyl cyclase”, Pharmacology and Therapeutics, 2018, 190, 173-186, and references cited therein; the entire contents of which is incorporated herein by reference.

In certain embodiments, the methods and uses described herein comprise administering to a subject a therapeutically effective amount of a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, a therapeutically effective amount is an amount sufficient for treating a disease or condition (e.g., ocular conditions (e.g., ocular hypotony), liver diseases (e.g., non-alcoholic steatohepatitis (NASH)), inflammatory diseases, autoimmune diseases (e.g., psoriasis)) in a subject. In certain embodiments, a therapeutically effective amount is an amount sufficient for contraception (e.g., male or female contraception). In certain embodiments, a therapeutically effective amount is an amount effective for inhibiting the activity of a sAC enzyme in a subject.

In certain embodiments, the methods and uses described herein comprise administering to a subject a prophylactically effective amount of a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, a prophylactically effective amount is an amount sufficient for preventing a disease or condition (e.g., ocular conditions (e.g., ocular hypotony), liver diseases (e.g., non-alcoholic steatohepatitis (NASH)), inflammatory diseases, autoimmune diseases (e.g., psoriasis)) in a subject. In certain embodiments, a prophylactically effective amount is an amount sufficient for preventing fertilization or pregnancy in a subject (i.e., contraception). In certain embodiments, a prophylactically effective amount is an amount sufficient for preventing the development, worsening, or progression of NASH in a subject. In certain embodiments, a prophylactically effective amount is an amount sufficient for inhibiting the activity of a sAC enzyme in a subject.

In certain embodiments, the subject or patient to be treated is a human. In certain embodiments, the subject or patient is a non-human mammal. In certain embodiments, the subject or patient is a dog.

### Contraception

As described herein, compounds and pharmaceutical compositions described herein are useful as male and/or female contraceptive agents. It is understood that in sperm, sAC is a major cAMP-generating enzyme crucial for sperm motility and capacitation. Capacitation is the essential maturation process required for sperm to acquire fertilization competence, commencing upon ejaculation and continues as sperm transit through the female reproductive tract. Without wishing to be bound by a particular theory, compounds described herein act as contraceptive agents by inhibiting sAC activity, thereby preventing capacitation of sperm and fertilization.

Provided herein are methods for male contraception, the methods comprise administering to a male subject a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use in male contraception. Also provided herein are uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of medicaments for male contraception.

In certain embodiments, the methods, compounds, and uses for male contraception comprise administering the compound or pharmaceutical composition orally to the male subject. In certain embodiments, the methods, compounds, and uses for male contraception comprise administering the compound or pharmaceutical composition orally to the male subject prior to intercourse. In certain embodiments, the administering is within less than 1 hour prior to intercourse. In certain embodiments, the administering is within about 1-24 hours prior to intercourse. In certain embodiments, the administering is within about 1, 2, 3, 4, 5, 6, 7, 8, 9, 10, 11, 12, 13, 14, 15, 16, 17, 18, 19, 20, 21, 22, 23, or 24 hours prior to intercourse. In certain embodiments the administering is within about 1-48 hours prior to intercourse. In certain embodiments the administering is within about 1 hour to 1 week prior to intercourse.

In certain embodiments, the administration is carried out regularly. In certain embodiments, the administration is carried out as needed prior to intercourse.

In another aspect, provided herein are methods for female contraception, the methods comprising administering to a female subject a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use in female contraception. Also provided herein are uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of medicaments for female contraception.

In certain embodiments, the methods and uses for female contraception comprise administering the compound or pharmaceutical composition intravaginally to a female subject (e.g., via intravaginal ring or film). In certain embodiments, the methods and uses for female contraception comprise administering the compound or pharmaceutical composition intravaginally to the female subject (e.g., via intravaginal ring or film) prior to intercourse. In certain embodiments, the methods comprise administering the contraceptive agent in the form of an intravaginal ring, film, cream, gel, foam, or lubricant to the female subject.

In certain embodiments, the methods, compounds, and uses for female contraception comprise administering the compound or pharmaceutical composition orally to the female subject. In certain embodiments, the methods, compounds, and uses for female contraception comprise administering the compound or pharmaceutical composition orally to the female subject prior to intercourse. In certain embodiments, the administering is within less than 1 hour prior to intercourse. In certain embodiments, the administering is within about 1-24 hours prior to intercourse. In certain embodiments, the administering is within about 1, 2, 3, 4, 5, 6, 7, 8, 9, 10, 11, 12, 13, 14, 15, 16, 17, 18, 19, 20, 21, 22, 23, or 24 hours prior to intercourse. In certain embodiments the administering is within about 1-48 hours prior to intercourse. In certain embodiments the administering is within about 1 hour to 1 week prior to intercourse.

In certain embodiments, the methods, compounds, and uses for female contraception comprise administering the compound or pharmaceutical composition orally to the female subject after intercourse (i.e., post-intercourse). In certain embodiments, the administering is within less than 1 hour post-intercourse, i.e., within less than 1-60 minutes post-intercourse. In certain embodiments, the administering is within about 1-24 hours post-intercourse.

For example, the compound can be administered orally to a female either before intercourse or after intercourse to prevent fertilization of an egg. If taken by a female before intercourse or within a period of time after intercourse (e.g., within minutes or hours), an orally delivered sAC inhibitor can be effective in blocking ejaculated sperm from reaching and fertilizing an egg in the reproductive tract of the female.

In certain embodiments, the administration is carried out regularly. In certain embodiments, the administration is carried out as needed prior to intercourse. In certain embodiments, the administration is carried out as needed post-intercourse.

In certain embodiments, compounds provided herein are administered to both a male and a female subject prior to intercourse. The compounds may be the same compound or different compounds provided herein. For example, a compound with a relatively longer off-rate may be administered to the male, while a compound with better female reproductive tissue penetration may be administered to the female. In this regard, in certain embodiments, provided herein are kits comprising “couples pills.” In certain embodiments, provided herein are kits comprising: (i) an oral contraceptive pill for administration to a male comprising a compound provided herein, or a pharmaceutically acceptable salt, solvate, hydrate, polymorph, co-crystal, tautomer, stereoisomer, isotopically labeled derivative, or prodrug thereof, or a pharmaceutical composition thereof; and (ii) an oral contraceptive pill for administration to a female comprising a compound provided herein, or a pharmaceutically acceptable salt, solvate, hydrate, polymorph, co-crystal, tautomer, stereoisomer, isotopically labeled derivative, or prodrug thereof, or a pharmaceutical composition thereof. Optionally, the kit comprises instructions for use.

### Ocular Conditions and Increasing Intraocular Pressure (IOP)

As described herein, compounds and pharmaceutical compositions described herein are useful for treating ocular conditions (e.g., ocular hypotony). Inhibition of sAC has been found to be a target for increasing intraocular pressure (IOP), which can affect the development and progression of various ocular conditions. Without wishing to be bound by a particular theory, compounds described herein inhibit sAC activity, leading to an increase in IOP. In turn, diseases or conditions that benefit from increasing intraocular pressure (IOP) (e.g., ocular hypotony) can be treated.

Provided herein are methods for treating an ocular condition (e.g., ocular hypotony) in a subject, the methods comprising administering to the subject a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use in treating an ocular condition (e.g., ocular hypotony). Also provided herein are uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of medicaments for treating ocular conditions (e.g., ocular hypotony). In certain embodiments, the ocular condition is ocular hypotony.

Provided herein are methods for increasing intraocular pressure (IOP) in the eye of a subject, the methods comprising administering to the subject a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use increasing intraocular pressure (IOP) in the eye of a subject.

Also provided herein are uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of medicaments for increasing intraocular pressure (IOP) in the eye of a subject.

In certain embodiments, the methods, compounds, and uses for treating ocular conditions (e.g., ocular hypotony) and/or increasing intraocular pressure (IOP) in the eye of a subject comprise administering the compound or pharmaceutical compostions to the eye of a subject (i.e., via ocular administration). In certain embodiments, the compound or pharmaceutical composition is administered topically to the eye (e.g., via eye drops). In certain embodiments, the compound or pharmaceutical composition is administered to the eye via intraocular injection. The compounds and pharmaceutical compositions provided herein can also be used to keep IOP elevated during or after procedures involving the eye (e.g., ocular surgery).

For example, in certain embodiments, a compound or pharmaceutical composition can be administered after glaucoma surgery (e.g., to prevent ocular pressure from falling too low until healing is complete).

### Liver Diseases

As described herein, compounds and pharmaceutical compositions described herein are useful for treating and/or preventing liver diseases (e.g., non-alcoholic steatohepatitis (NASH)). Soluble adenylyl cyclase (sAC) plays a role in the conversion of non-alcoholic fatty liver disease (NAFLD) into non-alcoholic steatohepatitis (NASH). NAFLD is becoming the most prevalent liver disease, and there are currently no approved pharmacotherapies. Without wishing to be bound by a particular theory, compounds provided herein can be used to treat and/or prevent NASH by inhibiting sAC activity, thereby preventing the conversion of NAFLD into NASH. In certain embodiments, the compounds and compositions can be used to prevent a liver disease (e.g., NASH) in a subject. In certain embodiments, the compounds and compositions can be used to prevent the development of NASH in subjects with NAFLD. In certain embodiments, the compounds and compositions can be used to prevent the worsening or progression of NASH in subjects.

Provided herein are methods for treating and/or preventing a liver disease (e.g., non-alcoholic steatohepatitis (NASH)) in a subject, the methods comprising administering to the subject a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use in treating and/or preventing a liver disease (e.g., non-alcoholic steatohepatitis (NASH)). Also provided herein are uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of medicaments for treating and/or preventing liver diseases (e.g., non-alcoholic steatohepatitis (NASH)).

In certain embodiments, the liver disease is NASH. In certain embodiments, the method, compound, or use is for preventing a liver disease (e.g., NASH) in a subject. In certain embodiments, the method, compound, or use is for preventing NASH in a subject. In certain embodiments, the method, compound, or use is for preventing the development of NASH in a subject with NAFLD. In certain embodiments, the method, compound, or use is for preventing the worsening or progression of NASH in a subject.

### Inflammatory Diseases and Autoimmune Diseases

As described herein, compounds and pharmaceutical compositions described herein are useful for treating inflammatory diseases and autoimmune diseases. Without wishing to be bound by any particular theory, it is believed that sAC plays a role in inflammation. For instance, inhibitors of sAC have been used to explore the role of cAMP in the regulation of the NLRP3-containing inflammasome, a key component leading to the maturation of the pro-inflammatory cytokine interleukin 10 (IL-1). As also described herein, sAC appears to be critical for Th17 cell activation and type 17 inflammation, and therefore sAC inhibitors can be used to treat Th17-mediated diseases, including inflammatory diseases and autoimmune diseases.

Provided herein are methods for treating an inflammatory disease in a subject, the methods comprising administering to the subject a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use in treating an inflammatory disease. Also provided herein are uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of medicaments for treating inflammatory diseases.

In certain embodiments, the inflammatory disease is a Th17-mediated inflammatory disease. In certain embodiments, the inflammatory disease involves type 17 inflammation.

As described herein, compounds and pharmaceutical compositions described herein are useful for treating autoimmune diseases. Provided herein are methods for treating an autoimmune disease in a subject, the methods comprising administering to the subject a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use in treating an autoimmune disease. Also provided herein are uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of medicaments for treating autoimmune diseases.

In certain embodiments, the autoimmune disease is a Th17-mediated autoimmune disease. In certain embodiments, the autoimmune disease involves a type 17 immune response.

Inhibitors of sAC described herein can be used to treat hyperproliferative diseases of the skin, including psoriasis, e.g., as described in U.S. Pat. No. 9,388,250; the entire contents of which is incorporated herein by reference. In certain embodiment, compounds and pharmaceutical compositions described herein are useful for treating psoriasis.

Provided herein are methods for treating psoriasis in a subject, the methods comprising administering to the subject a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. Also provided herein are compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use in treating psoriasis. Also provided herein are uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of medicaments for treating psoriasis.

In certain embodiments, the methods, compounds, and uses for treating psoriasis provided herein comprise administering to the subject a compound, or pharmaceutically acceptable salt thereof, topically (e.g., to the skin of the subject).

The compounds and compostions described herein are useful for treating other Th17-mediated diseases, including but not limited to, inflammatory bowel disease (IBD), multiple sclerosis (MS), and coronavirus disease (COVID). In certain embodiments, the disease is IBD. In certain embodiments, the disease is MS.

In certain embodiments, the disease is a disease associated with a cytokine storm, such as coronavirus disease (COVID). Without wishing to be bound by any particular theory, a sAC inhibitor described herein can prevent the expression of one or more cytokine storms typically associated with a COVID, and can therefore be used to treat and/or prevent COVID in a subject. In certain embodiments, a sAC inhibitor described herein can prevent the expression of one or more cytokine storms associated with the SARS-CoV-2 virus, and can therefore be used to treat and/or prevent COVID-19 in a subject.

### Inhibiting Soluble Adenylyl Cyclase

As described herein, compounds and pharmaceutical compositions described herein are useful for inhibiting the activity of soluble adenylyl cyclase (sAC) in a subject or biological sample.

Provided herein are methods for inhibiting the activity of soluble adenylyl cyclase (sAC) in a subject or biological sample, the methods comprising administering to the subject, or contacting the biological sample, with a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the inhibiting occurs in vivo in a subject. In certain embodiments, the inhibiting occurs in vitro in a biological sample.

Also provided herein are compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use in inhibiting the activity of soluble adenylyl cyclase (sAC) in a subject or biological sample. In certain embodiments, the inhibiting occurs in vivo in a subject. In certain embodiments, the inhibiting occurs in vitro in a biological sample.

Also provided herein are uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for the manufacture of a medicament for inhibiting the activity of soluble adenylyl cyclase (sAC) in a subject.

In certain embodiments, a compound provided herein has an off-rate (T_{1/2}) of greater than 20 seconds from a soluble adenylyl cyclase (sAC) protein. In certain embodiments, the compound has an off-rate of greater than about 20 seconds, 100 seconds, 500 seconds, 1,000 seconds, 2,000 seconds, 3,000 seconds, 4,000 seconds, 5,000 seconds, 6,000 seconds, 7,000 seconds, 8,000 seconds, 9,000 seconds, or 10,000 seconds. In certain embodiments, the compound has an off-rate of greater than about 10,000 seconds (e.g., from 10,000 seconds to 20,000 seconds). In certain embodiments, the compound has an off-rate of from 25-20,000 seconds, inclusive. In certain embodiments, the compound has an off-rate of from 1,000-20,000 seconds, inclusive. In certain embodiments, the compound has an off-rate of from 4,000-20,000 seconds, inclusive. In certain embodiments, the compound has an off-rate of from 25-10,000 seconds, inclusive. In certain embodiments, the compound has an off-rate of from 1,000-10,000 seconds, inclusive.

### EXAMPLES

### Synthesis of Compounds

### General Schemes

Examples can be prepared by routes known by those skilled in the art. For example, intermediate esters such as GS1.1 can be reacted with either EtOAc/NaH or LiHMDS/EtOAc to furnish keto-esters such as GS1.2. Keto-esters such as GS1.2 can be converted into pyrimidinones such as GS1.3 using guanidine carbonate in an appropriate solvent. Pyrimidnones such as GS1.3 can be converted into representative examples by treatment with dehydration reagents such as POCl₃.

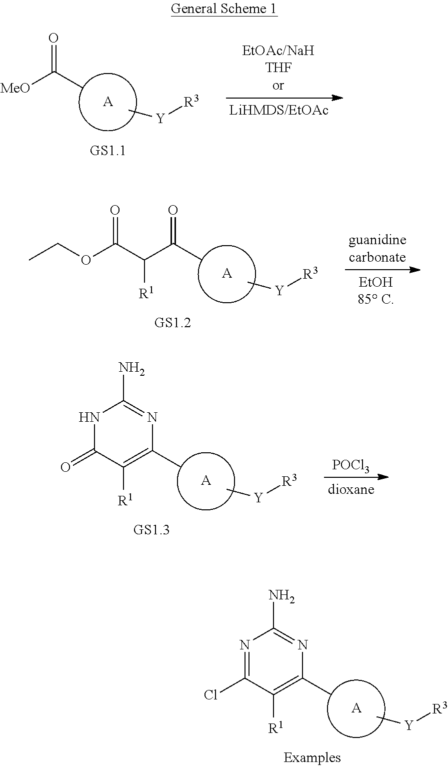

Intermediated esters such as GS1.1 can be prepared from appropriate halo-esters such as GS.2.1 via methods such as palladium catalyzed coupling with appropriate organometallic reagents as depicted in General Scheme 2. Esters such as GS1.1 can be converted into examples as depicted in General Scheme 1.

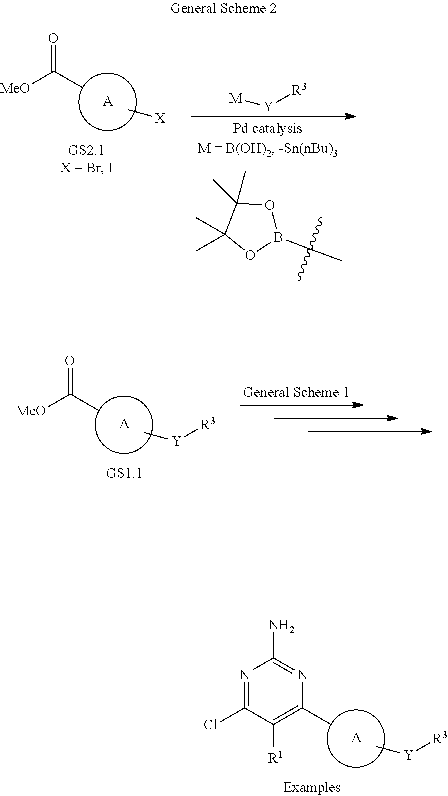

Halides such as GS2.1 can be metallated and reacted with aldedydes (R³CHO) to furnish alcohols such as GS3.1. The alcohol in GS3.1 can be reduced using standard conditions such as TMSCl/NaI or Et₃SiH/TFA to furnish intermediates such as GS3.2. Intermediates such as GS3.2 can be converted into examples where Y is —CH₂—. See General Scheme 3.

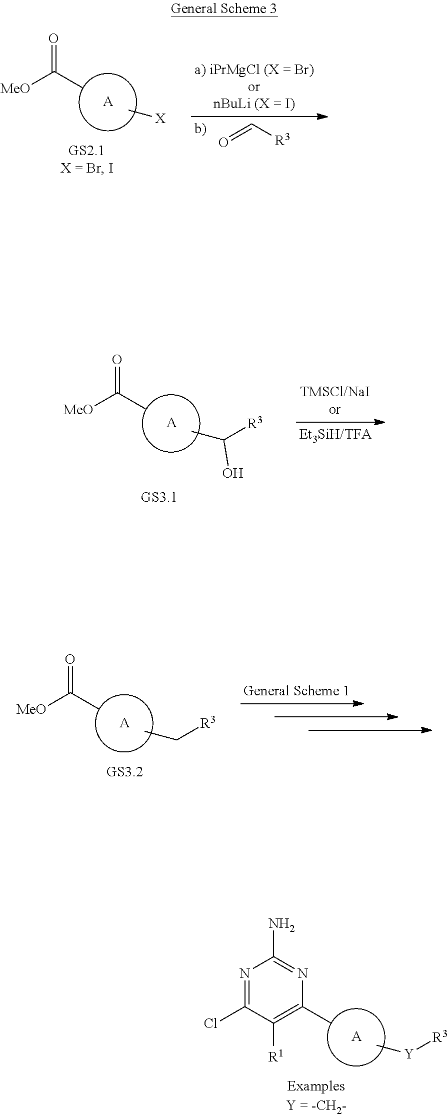

The following abbreviations are used in the synthetic routes: DCE (1,2-dichloroethane), THF (tetrahydrofuran), MeOH (methanol), DCM (dicholoromethane), Dess Martin periodinane (3-oxo-1,3-dihydro-1Δ⁵,2-benziodoxole-1,1,1-triyl triacetate), DMF (N,N-dimethylformamide), BINAP ((2,2′-bis(diphenylphosphino)-1,1′-binaphthyl)), ACN (acetonitrile), TEA (triethylamine), AcOH (acetic acid), EtOH (ethanol), EtOAc (ethyl acetate), DMAP (N,N-dimethylpyridin-4-amine), TFA (trifluoroacetic acid), HATU (1-[bis(dimethylamino)methylene]-1H-1,2,3-triazolo[4,5-b]pyridinium 3-oxide hexafluorophosphate), dba ((1E, 4E)-1,5-diphenylpenta-1,4-dien-3-one), NMO (4-methylmorpholine 4-oxide), FA (formic acid), DABCO (1,4-diazabicyclo[2.2.2]octane), CAN (ceric ammonium nitrate), dppf (1,1′-bis(diphenylphosphanyl) ferrocene), DME (1,2-dimethoxyethane), DCC (dicyclohexylmethanediimine), EDCI (3-(ethyliminomethyleneamino)-N,N-dimethylpropan-1-amine), HOBt (benzotriazol-1-ol), TFA (trifluoroacetic acid), TMSCl (chloro(trimethyl)silane), BPD [4,4,4′,4′, 5,5,5′,5′-Octamethyl-2,2′-bi(1,3,2-dioxaborolane)], LiHMDS (lithium 1,1,1-trimethyl-N-(trimethylsilyl)silanaminide), DIPEA (N-ethyl-N-(propan-2-yl)propan-2-amine), CDI (1,1′-carbonyldiimidazole), mCPBA (3-chlorobenzene-1-carboperoxoic acid), Xantphos (4,5-bis(diphenylphosphino)-9,9-dimethylxanthene), Phenofluor™ Mix (N,N′-1,3-bis(2,6-diisopropylphenyl)chloroimidazolium chloride/CsF), and PPh₃ (triphenylphosphine).

Preparative HPLC purification refers to the use of a water/acetonitrile gradient with or without the use of additives such as HCl, formic acid, TFA, or NH₄HCO₃ using an appropriate hydrophobic stationary phase.

In the table below, the CAS registry numbers are shown for the intermediates that are known in the literature and/or commercial. The preparation of Int I is depicted below.

Table of Intermediates
Int A

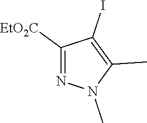

1373247-81-4
Int B

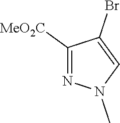

211738-66-8
Int C

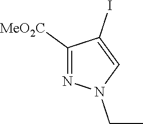

1354704-85-0
Int D

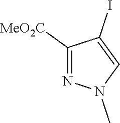

75092-25-0
Int E

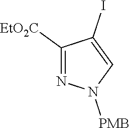

1355249-29-4
Int F

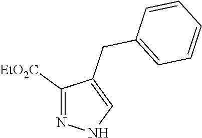 [SMILES: CCOC(=O)c1n[nH]cc1Cc1ccccc1]

1583555-36-5
Int G

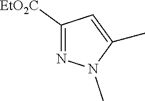

5744-51-4
Int H

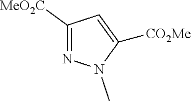

33146-99-5
Int I

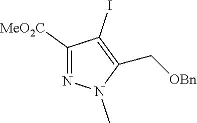 [SMILES: COC(=O)c1nn(C)c(COCc2ccccc2)c1I]

(synthesis described)
Int J

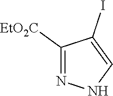 [SMILES: CCOC(=O)c1n[nH]cc1I]

179692-08-1
Int K

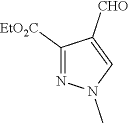

1594890-30-8
Int L

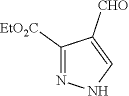 [SMILES: CCOC(=O)c1n[nH]cc1C=O]

179692-09-2
Int M

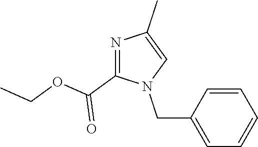 [SMILES: CCOC(=O)c1nc(C)cn1Cc1ccccc1]

1627504-34-0
Int N

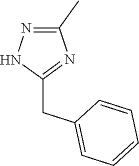

16194-97-1

### Aldehyde A

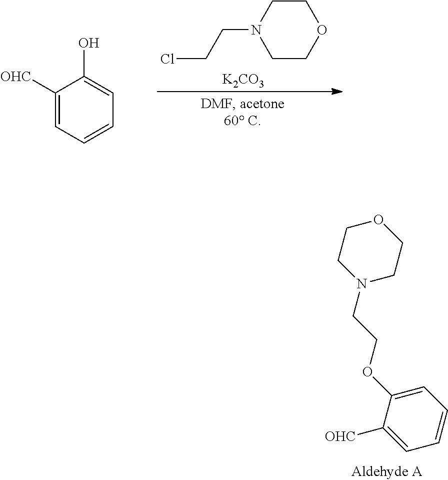

4-(2-chloroethyl)morpholine hydrochloride salt (7.62 g, 40.9 mmol) was added to a solution of 2-hydroxybenzaldehyde (5.00 g, 40.9 mmol, 4.35 mL, 1 eq) and K₂CO₃ (11.3 g, 81.9 mmol, 2 eq) in DMF (70 mL) and acetone (70 mL). The reaction mixture was heated at 60° C. for 12 h. The mixture was filtered and poured into 500 mL of water. The mixture was extracted with ethyl acetate (100 mL×4).

The organic layer was washed with 40 mL of an aqueous sodium hydroxide (0.1N) and then with 10 mL of brine. The reaction mixture was concentrated under the reduced pressure. The residue was purified by gradient flash chromatography (SiO₂, petroleum ether/ethyl acetate=1/1 to 1/0,) which furnished Aldehyde A.

### Halide A

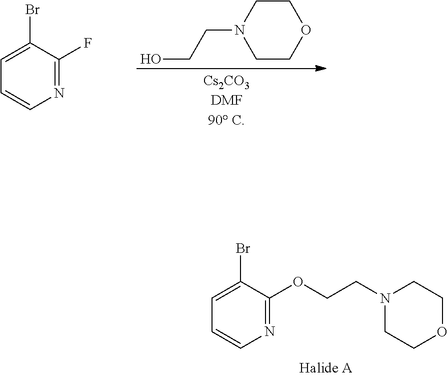 [SMILES: Fc1ncccc1Br ; OCCN1CCOCC1 ; Brc1cccnc1OCCN1CCOCC1]

To a mixture of 3-bromo-2-fluoro-pyridine (5.00 g, 28.4 mmol, 1 eq) and Cs₂CO₃ (18.5 g, 56.8 mmol, 2 eq) in DMF (50 mL) was added 2-morpholinoethanol (4.47 g, 34.1 mmol, 4.18 mL, 1.2 eq). The mixture was stirred at 90° C. for 12 h under N₂. The reaction mixture was diluted with water (200 mL). The mixture was extracted with EtOAc (100 mL×3). The organic layer was washed with brine (200 mL), dried over Na₂SO₄, and filtered. The filtrate was concentrated under reduced pressure. The residue was purified by flash chromatography (ISCO®; 40 g SepaFlash@ Silica Flash Column, gradient elution of 0 to 50% Ethyl acetate/petroleum ether @ 100 mL/min) which furnished 4-[2-[(3-bromo-2-pyridyl)oxy]ethyl]morpholine.

### Int I

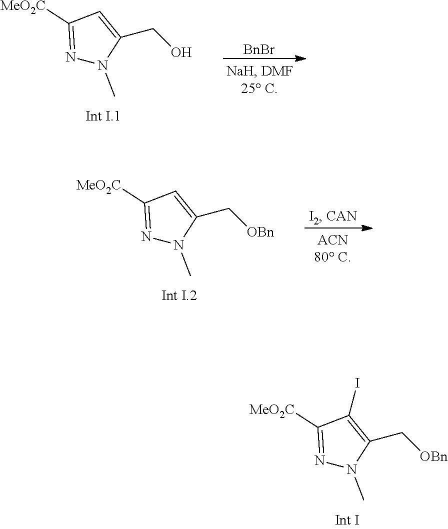

### Step 1

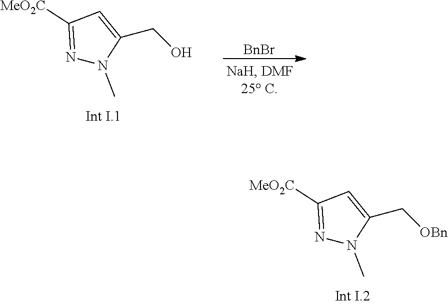 [SMILES: COC(=O)c1cc(CO)n(C)n1 ; "BnBr" ; COC(=O)c1cc(COCc2ccccc2)n(C)n1]

To a mixture of methyl 5-(hydroxymethyl)-1-methyl-pyrazole-3-carboxylate (2.30 g, 13.5 mmol, 1 eq) in DMF (30 mL) was added NaH (703 mg, 17.6 mmol, 60% wt % dispersion in oil, 1.3 eq) at 0° C. in portions. Benzyl bromide (3.47 g, 20.3 mmol, 2.41 mL, 1.5 eq) was added to the mixture. The mixture was stirred at 25° C. for 1 h under N₂. The reaction mixture was diluted with sat. aqueous NH₄Cl solution (100 mL). The solution was extracted with EtOAc (50 mL×3). The organic layer was washed with brine (100 mL), dried over Na₂SO₄, and filtered. The filtrate was concentrated under reduced pressure. The residue was purified by gradient flash chromatography (SiO₂, petroleum ether/ethyl acetate=4/1 to 3/2) which furnished Int 1.2.

### Step 2

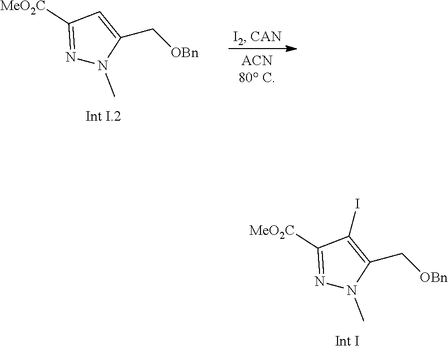

To a mixture of methyl 5-(benzyloxymethyl)-1-methyl-pyrazole-3-carboxylate (2.30 g, 8.84 mmol, 1 eq) in MeCN (25 mL) was added I₂ (1.35 g, 5.30 mmol, 0.6 eq). The mixture was stirred at 25° C. for 10 min. CAN (2.91 g, 5.30 mmol, 0.6 eq) was added to the mixture in portions, and the resulting mixture was stirred at 80° C. for 1 h. The reaction mixture was diluted with sat. aq. Na₂SO₃ solution (100 mL). The solution was extracted with EtOAc (50 mL×3). The organic layer was washed with brine (100 mL), dried over Na₂SO₄, and filtered. The filtrate was concentrated under reduced pressure. The residue was purified by gradient flash chromatography (SiO₂, petroleum ether/ethyl acetate=9/1 to 4/1) which furnished methyl 5-(benzyloxymethyl)-4-iodo-1-methyl-pyrazole-3-carboxylate.

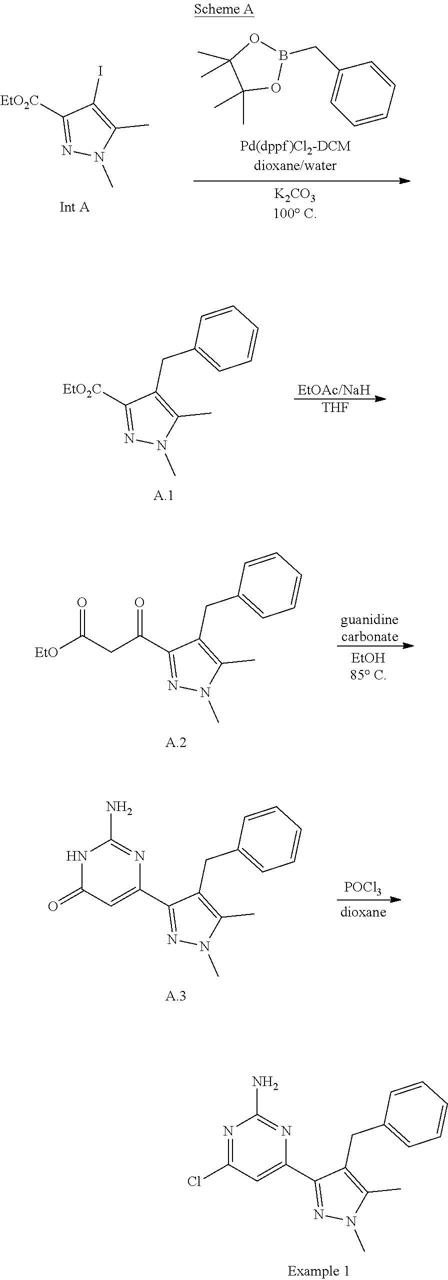

### Step 1

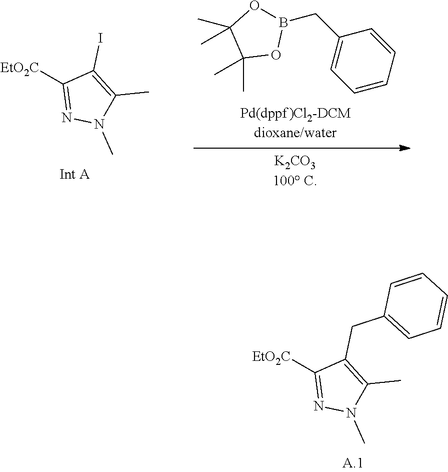 [SMILES: CCOC(=O)c1nn(C)c(C)c1I ; CC1(C)OB(Cc2ccccc2)OC1(C)C ; CCOC(=O)c1nn(C)c(C)c1Cc1ccccc1]

Ethyl 4-iodo-1,5-dimethyl-pyrazole-3-carboxylate (1.0 g, 3.4 mmol, 1 eq), 2-benzyl-4,4,5,5-tetramethyl-1,3,2-dioxaborolane (1.1 g, 5.0 mmol, 1.5 eq), Pd(dppf)Cl₂·CH₂Cl₂ (278 mg, 0.340 mmol, 0.1 eq) and K₂CO₃ (705 mg, 5.10 mmol, 1.5 eq) in dioxane (10 mL) and H₂O (2 mL) was de-gassed. The resulting mixture was heated at 100° C. for 12 hours under N₂. The reaction mixture was filtered through a pad of Celite. The filter cake was washed with EtOAc (20 mL×5). The filtrate was dried over Na₂SO₄, filtered, and concentrated under reduced pressure. The residue was purified by flash chromatography (ISCO®; 12 g SepaFlash® Silica Flash Column, gradient of 0-50% ethyl acetate in petroleum ether @ 75 mL/min) which furnished ethyl 4-benzyl-1,5-dimethyl-pyrazole-3-carboxylate (0.8 g).

### Step 2

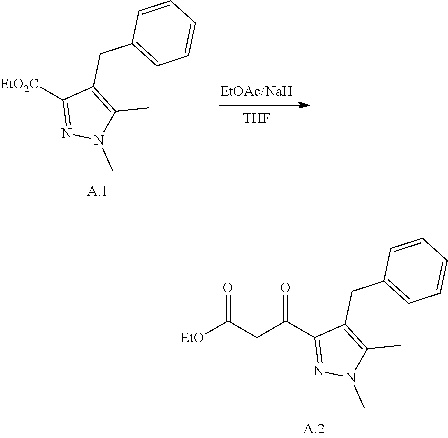 [SMILES: CCOC(=O)c1nn(C)c(C)c1Cc1ccccc1 ; CCOC(=O)CC(=O)c1nn(C)c(C)c1Cc1ccccc1 ; "THF"]

A mixture of ethyl 4-benzyl-1,5-dimethyl-pyrazole-3-carboxylate (800 mg, 3.10 mmol, 1 eq) in THF (10 mL) was cooled to 0° C. Sodium hydride (248 mg, 6.19 mmol, 60 wt % dispersion in oil, 2 eq) was added to the solution. After 20 min of stirring, EtOAc (1.91 g, 21.7 mmol, 2.1 mL, 7 eq) was added dropwise at 0° C. The mixture was stirred at 70° C. for 2 h under a N₂ atmosphere. The reaction mixture was poured into saturated NH₄Cl (aq.) (150 mL). The mixture was extracted with EtOAc (40 mL×3). The organic layers were washed with brine (100 mL), dried over Na₂SO₄ and filtered. The filtrate was concentrated under reduced pressure. The residue was purified by flash chromatography (ISCO®; 20 g SepaFlash® Silica Flash Column, gradient of 0-20% ethyl acetate in petroleum ether @ 75 mL/min) which furnished ethyl 3-(4-benzyl-1,5-dimethyl-pyrazol-3-yl)-3-oxo-propanoate (500 mg).

### Step 3

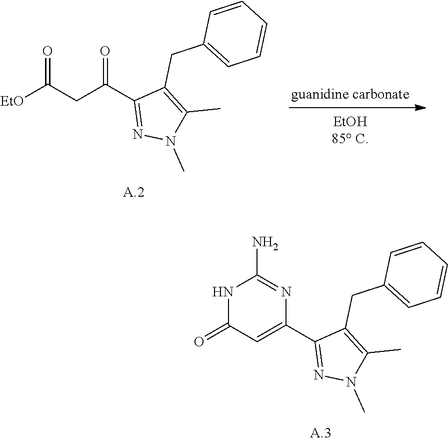 [SMILES: CCOC(=O)CC(=O)c1nn(C)c(C)c1Cc1ccccc1 ; "EtOH85° C." ; Cc1c(Cc2ccccc2)c(-c2cc(=O)[nH]c(N)n2)nn1C]

Ethyl 3-(4-benzyl-1,5-dimethyl-pyrazol-3-yl)-3-oxo-propanoate (500 mg, 1.66 mmol, 1 eq) and guanidine carbonate salt (900 mg, 4.99 mmol, 3 eq) were mixed in anhydrous EtOH (8 mL). The resulting mixture was stirred for 24 h at 85° C. under N₂. The reaction mixture was concentrated under reduced pressure to remove the EtOH. The residue was suspended in water (50 mL), and the solution was adjusted to pH=5 by addition of aq. HCL (1 N). The mixture was filtered, and the filter cake was washed with water (2 mL) and EtOH (2 mL). The collected solid was dried under reduced pressure which furnished amino-6-(4-benzyl-1,5-dimethyl-pyrazol-3-yl)-5H-pyrimidin-4-one.

### Step 4

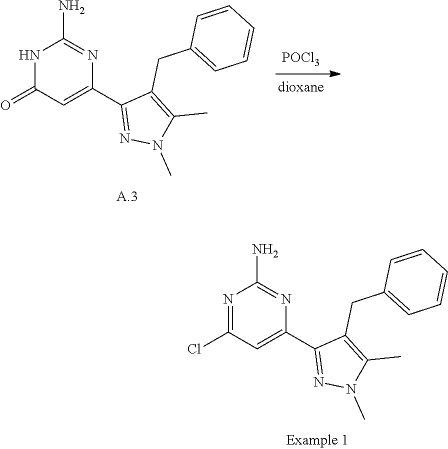

To a stirred solution of 2-amino-6-(4-benzyl-1,5-dimethyl-pyrazol-3-yl)-5H-pyrimidin-4-one (270 mg, 0.914 mmol, 1 eq) in dioxane (8 mL) was added POCl₃ (2.10 g, 13.7 mmol, 1.27 mL, 15 eq) dropwise at 20° C. The resulting mixture was heated at 75° C. for 12 h. Additional POCl₃ (2.10 g, 13.7 mmol, 1.27 mL, 15 eq) was added to the mixture. The resulting mixture was stirred for 6 h at 75° C. The reaction mixture was cooled and added slowly to aq. NaHCO₃ (saturated, 200 mL) to quench the excess POCl₃. The resulting solution was extracted with EtOAc (70 mL×3). The organic layer was washed with brine (100 mL), dried over Na₂SO₄ and filtered. The filtrate was concentrated under reduced pressure. The residue was purified by flash chromatography (ISCO®; 8 g SepaFlash® Silica Flash Column, gradient of 0-40% ethyl acetate in petroleum ether at 36 mL/min). The residue was further purified by neutral preparative-HPLC (Column: Waters Xbridge 150×25 mm, 5 m; mobile phase: [water (10 mM NH₄HCO₃)-ACN]; B %: 25%-55%, 10 min) which furnished 4-(4-benzyl-1,5-dimethyl-pyrazol-3-yl)-6-chloro-pyrimidin-2-amine Example 1. ¹H NMR: (400 MHz, DMSO-d₆): δ 7.18 (br s, 4H), 7.13-6.94 (m, 4H), 4.31 (br s, 2H), 3.78 (br s, 3H), 2.20 (br s, 3H) LCMS: (MH+) 314.1

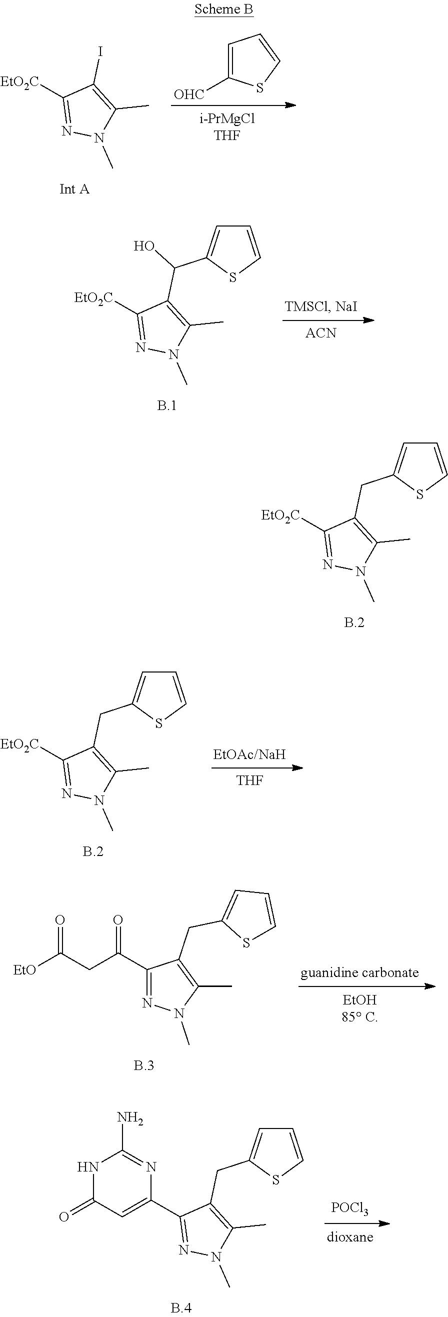

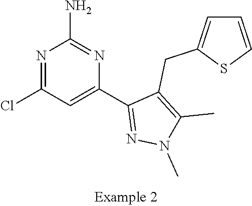 [SMILES: Cc1c(Cc2cccs2)c(-c2cc(Cl)nc(N)n2)nn1C]

### Step 1

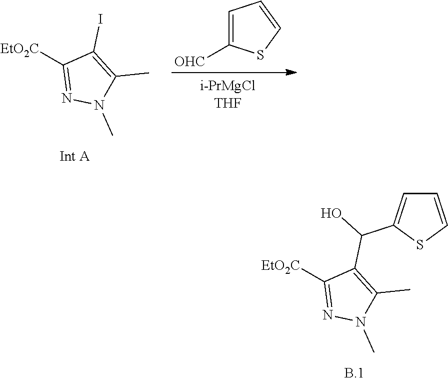 [SMILES: CCOC(=O)c1nn(C)c(C)c1I ; O=Cc1cccs1 ; CCOC(=O)c1nn(C)c(C)c1C(O)c1cccs1]

To a stirred solution of ethyl 4-iodo-1,5-dimethyl-pyrazole-3-carboxylate (0.600 g, 2.04 mmol, 1 eq) in THF (10 mL) was added isopropylmagnesium chloride-lithium chloride complex (1.3 M, 1.65 mL, 1.05 eq) at −10° C. under a N₂ atmosphere. After stirring for 0.5 h at −10° C., a solution of thiophene-2-carbaldehyde (252 mg, 2.24 mmol, 1.1 eq) in THF (1 mL) was added to the mixture dropwise. After the addition, the mixture was allowed to warm slowly to 15° C. and stirred at that temperature for 12 h. The reaction was diluted with sat. aq. NH₄Cl solution (100 mL), and the resulting mixture was extracted with EtOAc (50 mL*3). The combined organic layers were washed with brine (70 mL), dried over Na₂SO₄ and filtered. The filtrate was concentrated under reduced pressure. The residue was purified by column chromatography (SiO₂, petroleum ether/ethyl acetate=1/1) which furnished ethyl 4-[hydroxy(2-thienyl)methyl]-1,5-dimethyl-pyrazole-3-carboxylate.

### Step 2

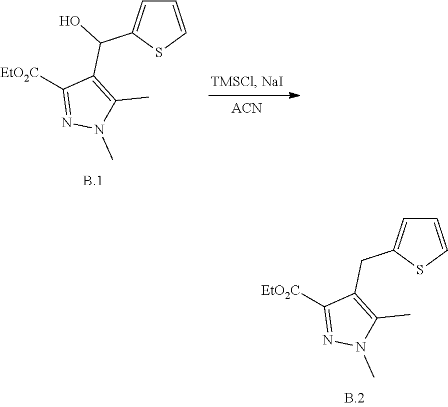 [SMILES: CCOC(=O)c1nn(C)c(C)c1C(O)c1cccs1 ; "ACN" ; CCOC(=O)c1nn(C)c(C)c1Cc1cccs1]

To a solution of NaI (1.28 g, 8.56 mmol, 6 eq) in ACN (6 mL) was added TMSCl (930 mg, 8.56 mmol, 1.09 mL, 6 eq) under N₂. After stirring at 15° C. for 10 minutes, a solution of ethyl 4-[hydroxy(2-thienyl)methyl]-1,5-dimethyl-pyrazole-3-carboxylate (400 mg, 1.43 mmol, 1 eq) in ACN (2 mL) was added. The mixture was stirred at 15° C. under N₂ for 2 h. The reaction mixture was diluted with sat. aq Na₂SO₃ (70 mL). The solution was extracted with EtOAc (50 mL×3). The combined organic layers were washed with brine (100 mL), dried over Na₂SO₄, and filtered. The filtrate was concentrated under the reduced pressure. The residue was purified by column chromatography (SiO₂, petroleum ether/ethyl acetate=1/1) which furnished ethyl 1,5-dimethyl-4-(2-thienylmethyl)pyrazole-3-carboxylate.

### Example 2

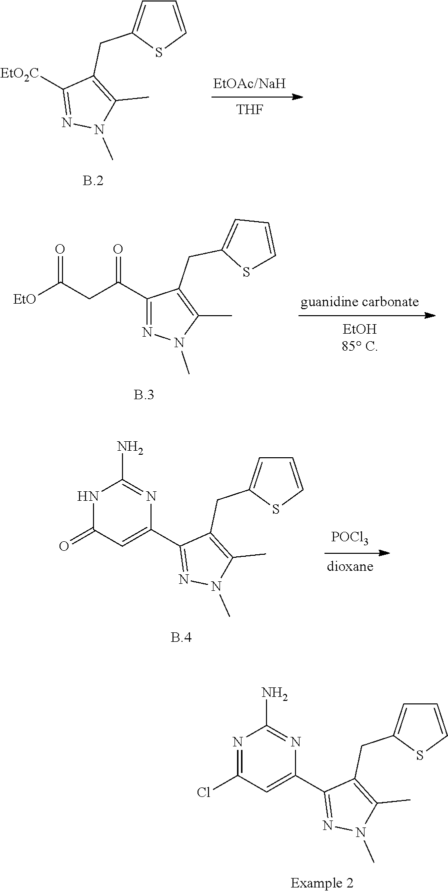

Ethyl 1,5-dimethyl-4-(2-thienylmethyl)pyrazole-3-carboxylate B.2 was converted into 4-chloro-6-[1,5-dimethyl-4-(2-thienylmethyl)pyrazol-3-yl] pyrimidin-2-amine Example 2 using conditions similar to that outlined for the transformation of A.1 into Example 1 (Scheme A). Example 2: ¹H NMR: (400 MHz, DMSO-d₆): δ 7.18 (dd, J=1.2, 5.0 Hz, 1H), 7.04 (br s, 2H), 6.98 (s, 1H), 6.89-6.86 (m, 1H), 6.83 (dd, J=3.4, 5.0 Hz, 1H), 4.49 (s, 2H), 3.78 (s, 3H), 2.24 (s, 3H); LCMS: (MH+) 320.0.

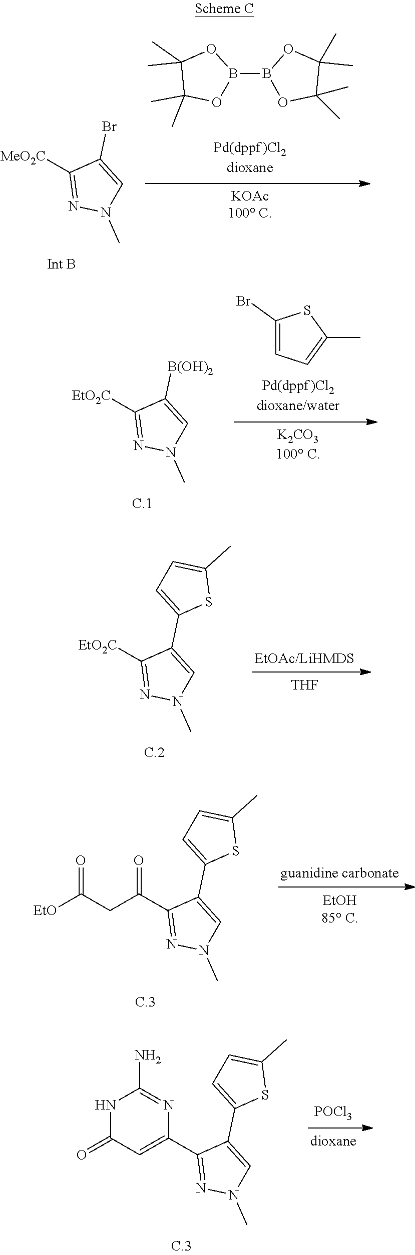

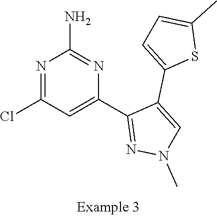 [SMILES: Cc1ccc(-c2cn(C)nc2-c2cc(Cl)nc(N)n2)s1]

### Step 1

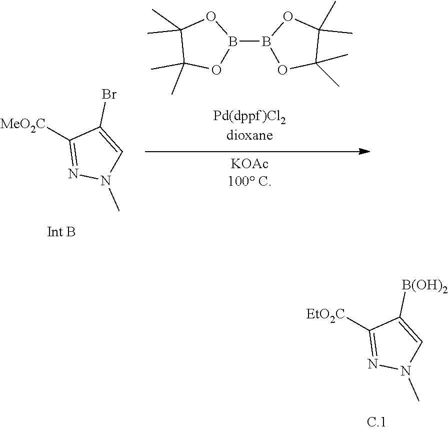

Methyl 4-bromo-1-methyl-pyrazole-3-carboxylate (5.0 g, 23 mmol, 1 eq), BPD (6.4 g, 25 mmol, 1.1 eq), Pd(dppf)Cl₂ (835 mg, 1.14 mmol, 0.05 eq) and KOAc (4.48 g, 45.7 mmol, 2 eq) in dioxane (80 mL) was de-gassed. The resulting mixture was heated to 100° C. for 12 h under N₂. The reaction mixture was filtered, and the filtrate was concentrated under reduced pressure. The residue was purified by column chromatography (SiO₂, petroleum ether/ethyl acetate gradient=4/1 to 1/1) to give (3-methoxycarbonyl-1-methyl-pyrazol-4-yl)boronic acid.

### Step 2

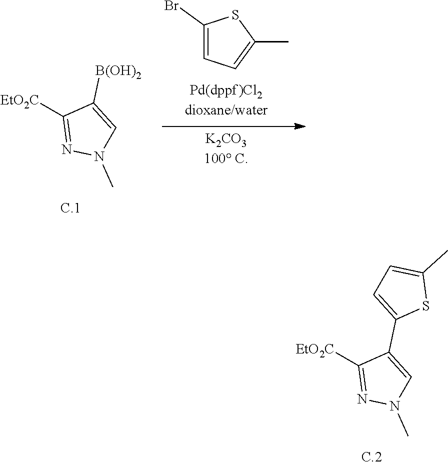

A mixture of (3-methoxycarbonyl-1-methyl-pyrazol-4-yl)boronic acid (4.7 g, 26 mmol, 1 eq), 2-bromo-5-methyl-thiophene (6.8 g, 38 mmol, 4.4 mL, 1.5 eq), Pd(dppf)Cl₂ (1.87 g, 2.56 mmol, 0.1 eq) and K₂CO₃ (7.06 g, 51.1 mmol, 2 eq) in dioxane (50 mL)/H₂O (10 mL) was de-gassed. The resulting mixture was heated to 80° C. for 12 h under N₂. The reaction mixture was diluted with water (200 mL). The solution was extracted with EtOAc (50 mL×4). The organic layer was washed with brine (100 mL), dried over Na₂SO₄, and filtered. The filtrate was concentrated under reduced pressure. The residue was purified by column chromatography (SiO₂, petroleum ether/ethyl acetate gradient=5/1 to 2/1) to furnish methyl 1-methyl-4-(5-methyl-2-thienyl)pyrazole-3-carboxylate.

### Step 3

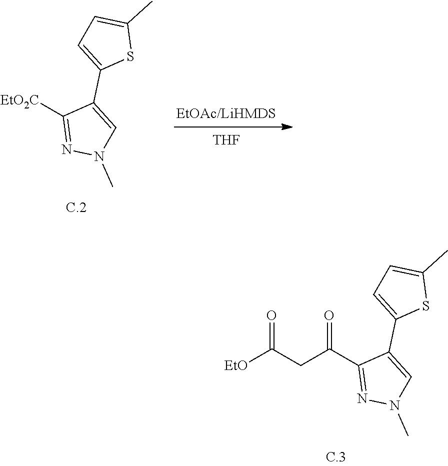 [SMILES: CCOC(=O)c1nn(C)cc1-c1ccc(C)s1 ; "THF" ; CCOC(=O)CC(=O)c1nn(C)cc1-c1ccc(C)s1]

Methyl 1-methyl-4-(5-methyl-2-thienyl)pyrazole-3-carboxylate (800 mg, 3.39 mmol, 1 eq) and EtOAc (2.09 g, 23.7 mmol, 2.32 mL, 7 eq) were mixed in THF (15 mL). After the solution was cooled to −40° C., LiHMDS (1 M, 10.16 mL, 3 eq) was added in one portion. The mixture was stirred at −40° C. for 2 h. The reaction mixture was added slowly to an aq. sat. NH₄Cl solution (150 mL). The solution was extracted with EtOAc (30 mL×4). The organic layer was washed with brine (40 mL), dried over Na₂SO₄, and filtered. The filtrate was concentrated under reduced pressure. The residue was purified by column chromatography (SiO₂, petroleum ether/ethyl acetate gradient=10/1 to 6/1) to furnish ethyl 3-[1-methyl-4-(5-methyl-2-thienyl)pyrazol-3-yl]-3-oxo-propanoate.

### Example 3

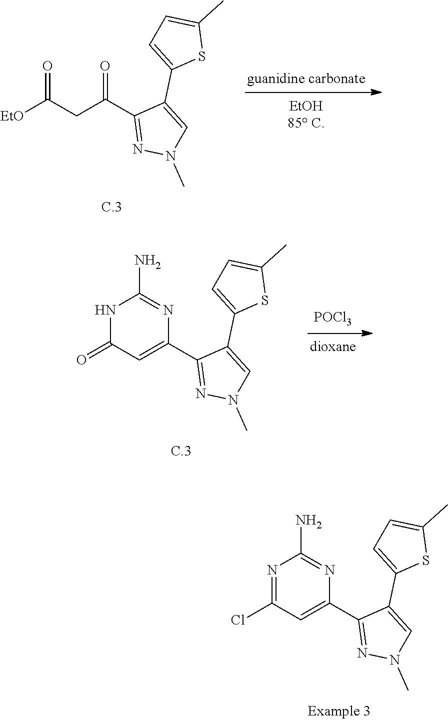

The intermediate C.3 was converted into Example 3 using conditions similar to that outlined in Scheme A (Steps 3 and 4). Example 3: ¹H NMR: (400 MHz, CD3OD) δ 7.49 (s, 1H), 6.97 (s, 1H), 6.92 (d, J=3.4 Hz, 1H), 6.69 (dd, J=1.0, 3.4 Hz, 1H), 5.31 (br d, J=3.3 Hz, 2H), 3.99 (s, 3H), 2.49 (s, 3H); LCMS: (MH+) 306.0.

The following examples in Table 1 were prepared in a similar fashion to Example 3 using the appropriate reagent/conditions in Step 2 of Scheme C.

TABLE 1

| | | Reagent/ | 1H NMR (400 | LCMS |
|---|---|---|---|---|
| Ex. | Structure | Conditions | MHz) | (MH+) |
| 4 | 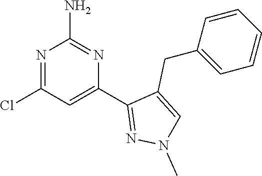 [SMILES: Cn1cc(Cc2ccccc2)c(-c2cc(Cl)nc(N)n2)n1] | 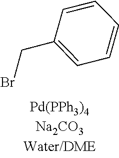 | (DMSO-d6) δ 7.50 (s, 1H), 7.29-7.21 (m, 4H), 7.17-7.11 (m, 1H), 7.08 (s, 2H), 6.99 (s, 1H), 4.27 (s, 2H), 3.84 (s, 3H) | 300.0 |
| 30 | 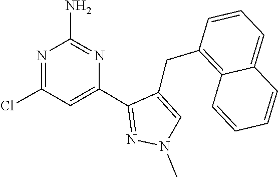 [SMILES: Cn1cc(Cc2cccc3ccccc23)c(-c2cc(Cl)nc(N)n2)n1] | 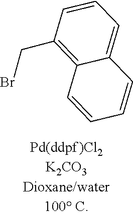 [SMILES: BrCc1cccc2ccccc12] | (DMSO-d₆) δ 8.04 (br d, J = 7.1 Hz, 1H), 7.94- 7.88 (m, 1H), 7.83-7.77 (m, 1H), 7.52-7.41 (m, 4H), 7.09 (br s, 3H), 7.05 (s, 1H), 4.74 (s, 2H), 3.74 (s, 3H) | 350.0 |
| 31 | 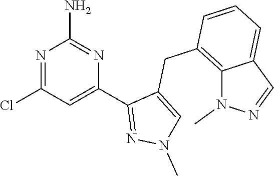 [SMILES: Cn1cc(Cc2cccc3cnn(C)c23)c(-c2cc(Cl)nc(N)n2)n1] | 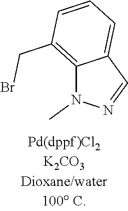 | (DMSO-d6) δ 8.00 (s, 1H), 7.64 (d, J = 8.0 Hz, 1H), 7.16 (br d, J = 6.9 Hz, 1H), 7.11 (br s, 2H), 7.09-7.03 (m, 2H), 7.00 (s, 1H), 4.77 (s, 2H), 4.05 (s, 3H), 3.77 (s, 3H) | 354.1 |
| 32 | 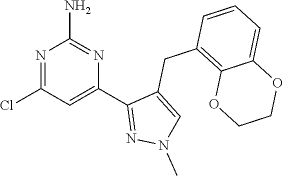 [SMILES: Cn1cc(Cc2cccc3c2OCCO3)c(-c2cc(Cl)nc(N)n2)n1] | 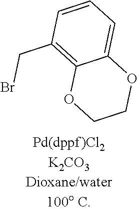 [SMILES: BrCc1cccc2c1OCCO2] | (DMSO-d₆) δ 7.29-7.24 (m, 1H), 7.10-7.03 (m, 2H), 6.99 (s, 1H), 6.73-6.67 (m, 3H), 4.26- 4.20 (m, 4H), 4.16 (s, 2H), 3.83-3.79 (m, 3H) | 358.1 |

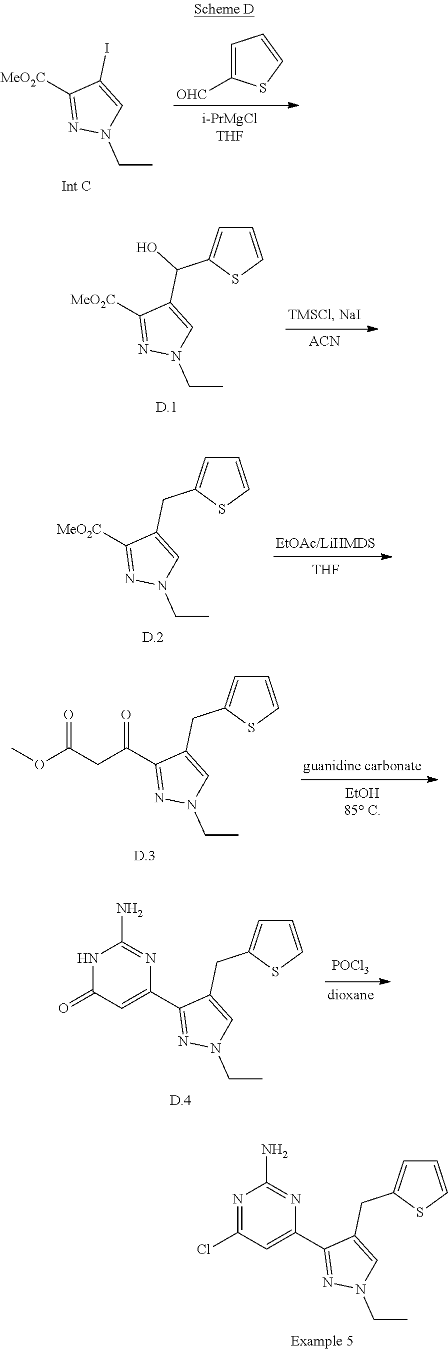

Example 5 was prepared from Int C in a similar fashion to that described. Int C was converted into D.2 using conditions outlined in Scheme B (Step 1 and 2). D.2 was converted into Example 5 using conditions outlined in Scheme C (C.2 to Example 3).

Example 5: ¹H NMR: (400 MHz, DMSO-d₆) δ 7.65 (s, 1H), 7.25 (dd, J=1.3, 5.1 Hz, 1H), 7.07 (br s, 2H), 7.01 (s, 1H), 6.93-6.90 (m, 1H), 6.90-6.87 (m, 1H), 4.49 (s, 2H), 4.14 (q, J=7.2 Hz, 2H), 1.37 (t, J=7.3 Hz, 3H); LCMS: (MH+) 320.0

The following examples in Table 2 were prepared in a similar fashion to Example 5 in Scheme D using the appropriate Intermediate and aldehyde/ketone (Step 1).

TABLE 2

| | | Intermediate | 1H NMR | LCMS |
|---|---|---|---|---|
| Ex. | Structure | aldehyde/ketone | (400 MHz) | (MH+) |
| 6 | 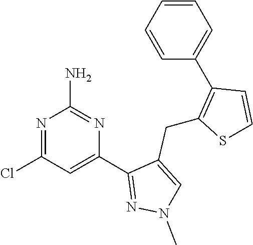 [SMILES: Cn1cc(Cc2sccc2-c2ccccc2)c(-c2cc(Cl)nc(N)n2)n1] | 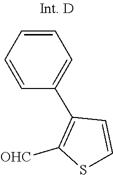 [SMILES: O=Cc1sccc1-c1ccccc1] | (DMSO-d6) δ 7.44 (s, 1H), 7.42-7.40 (m, 5H), 7.35- 7.28 (m, 1H), 7.11 (d, J = 5.14 Hz, 1H), 7.01 (s, 1H), 6.94 (br s, 2H), 4.49 (s, 2H), 3.84 (s, 3H) | 382.1 |
| 7 | 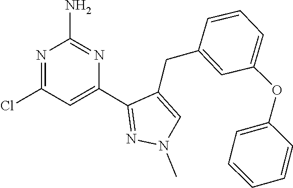 [SMILES: Cn1cc(Cc2cccc(Oc3ccccc3)c2)c(-c2cc(Cl)nc(N)n2)n1] | 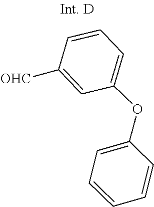 [SMILES: O=Cc1cccc(Oc2ccccc2)c1] | (CD₃CN, HCl salt) δ 7.40 (s, 1H), 7.38-7.32 (m, 2H), 7.28 (t, J = 7.76 Hz, 1H), 7.12 (t, J = 7.40 Hz, 1H), 7.01- 6.91 (m, 4H), 6.87-6.79 (m, 2H), 4.17 (s, 2H), 3.89 (s, 3H) | 392.1 |
| 8 | 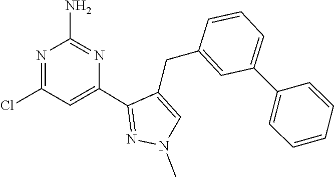 [SMILES: Cn1cc(Cc2cccc(-c3ccccc3)c2)c(-c2cc(Cl)nc(N)n2)n1] | 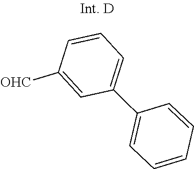 [SMILES: O=Cc1cccc(-c2ccccc2)c1] | (DMSO-d6, HCl salt) δ 7.65-7.58 (m, 4H), 7.47- 7.40 (m, 3H), 7.37-7.29 (m, 2H), 7.28- 7.23 (m, 1H), 6.99 (s, 1H), 4.34 (s, 2H), 3.84 (s, 3H) | 376.1 |
| 9 | 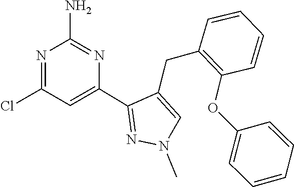 [SMILES: Cn1cc(Cc2ccccc2Oc2ccccc2)c(-c2cc(Cl)nc(N)n2)n1] | 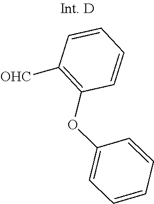 [SMILES: O=Cc1ccccc1Oc1ccccc1] | (DMSO-d6) δ 7.41-7.28 (m, 4H), 7.25- 7.17 (m, 1H), 7.12-7.00 (m, 4H), 6.94 (s, 1H), 6.87 (dd, J = 2.6, 7.9 Hz, 3H), 4.23 (s, 2H), 3.79 (s, 3H) | 392.1 |
| 10 | 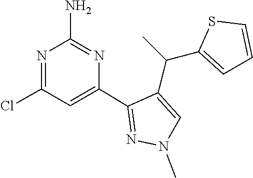 [SMILES: CC(c1cccs1)c1cn(C)nc1-c1cc(Cl)nc(N)n1] | 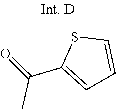 | (CD3OD) δ 7.49 (s, 1H), 7.12 (dd, J = 1.65, 4.58 Hz, 1H), 7.09 (s, 1H), 6.88- 6.83 (m, 2H), 5.38 (q, J = 7.13 Hz, 1H), 3.89 (s, 3H), 1.64 (d, J = 7.09 Hz, 3H) | 320.0 |
| | racemic | | | |
| 11 | 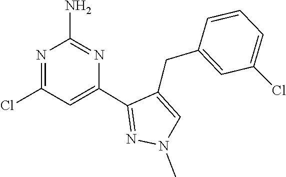 [SMILES: Cn1cc(Cc2cccc(Cl)c2)c(-c2cc(Cl)nc(N)n2)n1] | 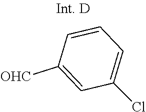 | (DMSO-d6) δ 7.61 (s, 1H), 7.34 (s, 1H), 7.22-7.29 (m, 2H), 7.17- 7.21 (m, 1H), 7.11 (s, 2H), 6.98 (s, 1H), 4.28 (s, 2H), 3.85 (s, 3H) | 334.0 |
| 12 | 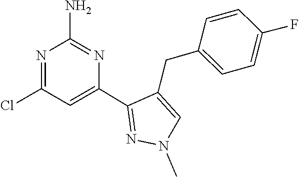 [SMILES: Cn1cc(Cc2ccc(F)cc2)c(-c2cc(Cl)nc(N)n2)n1] | 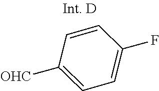 | (DMSO-d6) δ 7.53 (s, 1H), 7.30 (dd, J = 5.7, 8.4 Hz, 2H), 7.08 (br s, 2H), 7.07- 7.02 (m, 2H), 6.98 (s, 1H), 4.24 (s, 2H), 3.84 (s, 3H) | 318.1 |
| 13 | 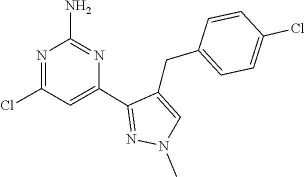 [SMILES: Cn1cc(Cc2ccc(Cl)cc2)c(-c2cc(Cl)nc(N)n2)n1] | 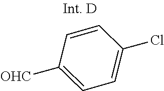 | (CD₃OD) δ 7.33 (s, 1H), 7.22 (s, 4H), 7.10 (s, 1H), 4.27 (s, 2H), 3.88 (s, 3H) | 334.0 |
| 14 | 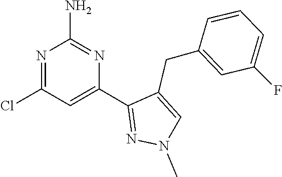 [SMILES: Cn1cc(Cc2cccc(F)c2)c(-c2cc(Cl)nc(N)n2)n1] | 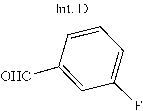 | (DMSO-d6) δ 7.59 (s, 1H), 7.32-7.23 (m, 1H), 7.10 (br s, 4H), 7.01- 6.91 (m, 2H), 4.28 (s, 2H), 3.85 (s, 3H) | 318.2 |
| 15 | 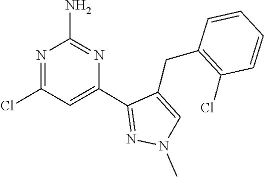 [SMILES: Cn1cc(Cc2ccccc2Cl)c(-c2cc(Cl)nc(N)n2)n1] | 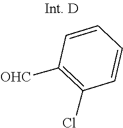 | (DMSO-d₆) δ 7.46-7.40 (m, 1H), 7.38- 7.34 (m, 1H), 7.29-7.22 (m, 3H), 7.07 (br s, 2H), 7.01 (s, 1H), 4.37 (s, 2H), 3.82 (s, 3H) | 334.0 |
| 16 | 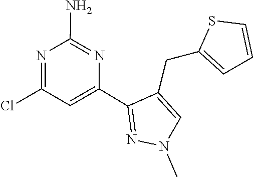 [SMILES: Cn1cc(Cc2cccs2)c(-c2cc(Cl)nc(N)n2)n1] | 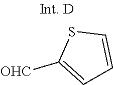 | (CD3OD) δ 7.42 (s, 1H), 7.18-7.10 (m, 2H), 6.91- 6.83 (m, 2H), 4.49 (s, 2H), 3.89 (s, 3H) | 306.0 |
| 17 | 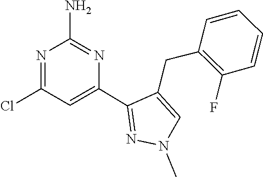 [SMILES: Cn1cc(Cc2ccccc2F)c(-c2cc(Cl)nc(N)n2)n1] | 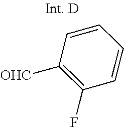 | (CD3OD) δ 7.28 (s, 1H), 7.26-7.16 (m, 2H), 7.12 (s, 1H), 7.05 (d, J = 8.2 Hz, 2H), 4.31 (s, 2H), 3.87 (s, 3H) | 318.1 |
| 19 | 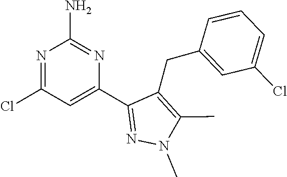 [SMILES: Cc1c(Cc2cccc(Cl)c2)c(-c2cc(Cl)nc(N)n2)nn1C] | 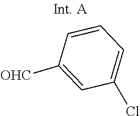 | (DMSO-d₆) δ 7.27-7.24 (m, 1H), 7.22 (d, J = 7.8 Hz, 1H), 7.17- 7.13 (m, 2H), 7.05 (s, 2H), 6.99 (s, 1H), 4.32 (s, 2H), 3.80 (s, 3H), 2.23 (s, 3H) | 348.0 |
| 20 | 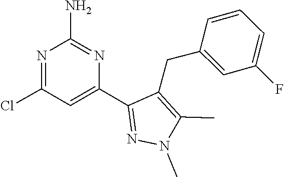 [SMILES: Cc1c(Cc2cccc(F)c2)c(-c2cc(Cl)nc(N)n2)nn1C] | 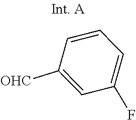 | (DMSO-d₆) δ 7.26-7.19 (m, 1H), 7.09- 7.00 (m, 4H), 6.98 (s, 1H), 6.91 (dt, J = 2.1, 8.5 Hz, 1H), 4.32 (s, 2H), 3.79 (s, 3H), 2.22 (s, 3H) | 332.1 |
| 21 | 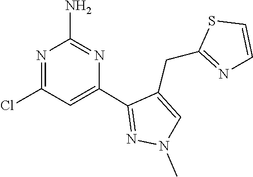 [SMILES: Cn1cc(Cc2nccs2)c(-c2cc(Cl)nc(N)n2)n1] | 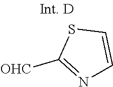 | (DMSO-d₆) δ 7.72 (s, 1H), 7.65 (d, J = 3.4 Hz, 1H), 7.49 (d, J = 3.4 Hz, 1H), 7.05 (br s, 2H), 7.01 (s, 1H), 4.69 (s, 2H), 3.87 (s, 3H) | 307.0 |
| 25 | 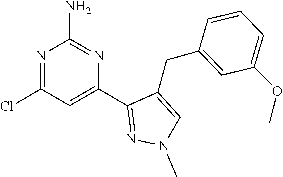 [SMILES: COc1cccc(Cc2cn(C)nc2-c2cc(Cl)nc(N)n2)c1] | 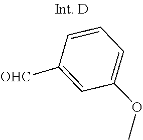 | (CD3OD) δ 7.30 (s, 1H), 7.14 (t, J = 7.8 Hz, 1H), 7.10 (s, 1H), 6.85-6.76 (m, 2H), 6.74- 6.67 (m, 1H), 4.25 (s, 2H), 3.88 (s, 3H), 3.73 (s, 3H) | 330.1 |
| 26 | 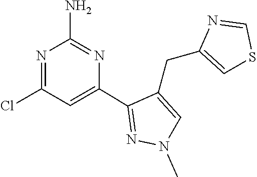 [SMILES: Cn1cc(Cc2cscn2)c(-c2cc(Cl)nc(N)n2)n1] | 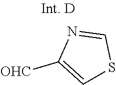 | (DMSO-d6) δ 8.99 (d, J = 1.9 Hz, 1H), 7.53 (s, 1H), 7.39 (d, J = 1.8 Hz, 1H), 7.10 (s, 2H), 7.00 (s, 1H), 4.42 (s, 2H), 3.85 (s, 3H) | 307.0 |
| 27 | 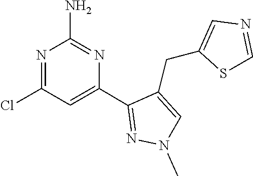 [SMILES: Cn1cc(Cc2cncs2)c(-c2cc(Cl)nc(N)n2)n1] | 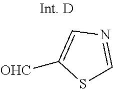 | (DMSO-d6) δ 8.84 (s, 1H), 7.76 (s, 1H), 7.67 (s, 1H), 7.12 (br s, 2H), 6.99 (s, 1H), 4.52 (s, 2H), 3.85 (s, 3H) | 307.1 |
| 28 | 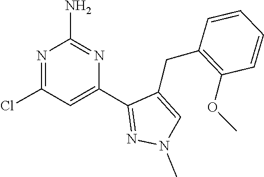 [SMILES: COc1ccccc1Cc1cn(C)nc1-c1cc(Cl)nc(N)n1] | 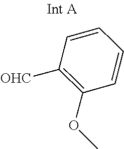 | (DMSO-d6) δ 7.23 (s, 1H), 7.20-7.14 (m, 2H), 7.07 (br s, 2H), 6.98 (s, 1H), 6.96 (d, J = 7.9 Hz, 1H), 6.83 (t, J = 7.4 Hz, 1H), 4.19 (s, 2H), 3.80 (s, 3H), 3.76 (s, 3H) | 330.1 |
| 43 | 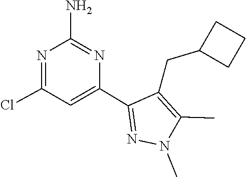 [SMILES: Cc1c(CC2CCC2)c(-c2cc(Cl)nc(N)n2)nn1C] | 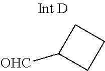 | (DMSO-d₆, 400 MHz) δ 6.97 (s, 1H), 6.96-6.92 (m, 2H), 3.77 (s, 3H), 2.94 (d, J = 7.25 Hz, 2H), 2.46- 2.41 (m, 1H), 2.21 (s, 3H), 1.87-1.78(m, 2H), 1.75- 1.53 (m, 4H) | 292.2 |
| 69 | 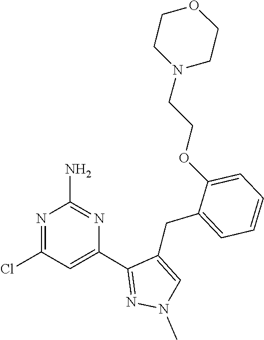 [SMILES: Cn1cc(Cc2ccccc2OCCN2CCOCC2)c(-c2cc(Cl)nc(N)n2)n1] | 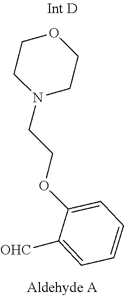 [SMILES: O=Cc1ccccc1OCCN1CCOCC1] | (DMSO-d6) δ 8.13 (s, 1H), 7.26 (s, 1H), 7.21 (dd, J = 1.6, 7.4 Hz, 1H), 7.19- 7.12 (m, 1H), 7.06 (br s, 2H), 6.98 (s, 1H), 6.95 (d, J = 8.0 Hz, 1H), 6.84 (t, J = 7.3 Hz, 1H), 4.20 (s, 2H), 4.05 (t, J = 5.6 Hz, 2H), 3.80 (s, 3H), 3.53-3.48 (m, 4H), 2.63 (br t, J = 5.3 Hz, 2H), 2.40 (br s, 4H) | 429.2 |

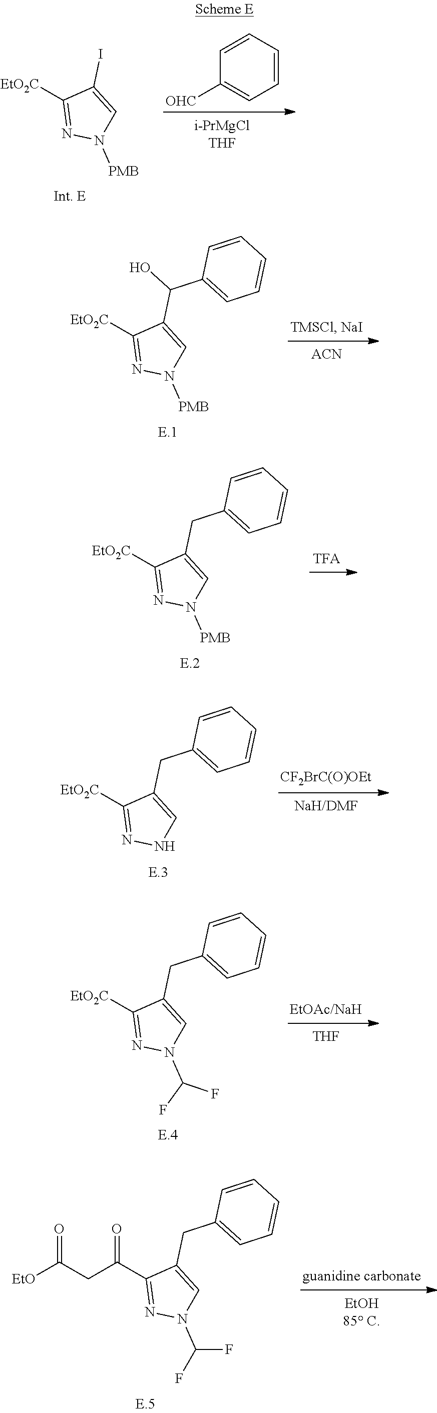

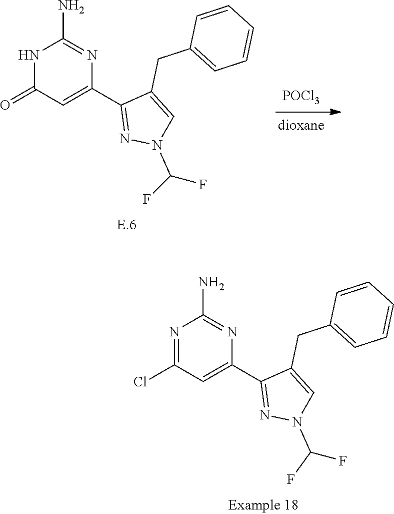

### Step 1

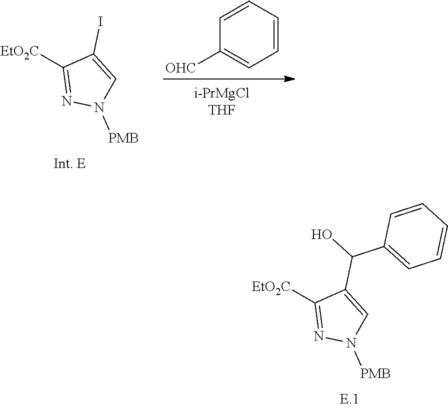

To a mixture of ethyl 4-iodo-1-[(4-methoxyphenyl)methyl]pyrazole-3-carboxylate (3.0 g, 7.8 mmol, 1 eq) in THF (30 mL) was added i-PrMgCl·LiCl (1.3 M, 6.3 mL, 1.05 eq) dropwise at −15° C. under N₂. After stirring at −15° C. for 30 minutes, benzaldehyde (907 mg, 8.55 mmol, 864 uL, 1.1 eq) was added to the mixture dropwise at −15° C. The resulting reaction mixture was stirred at 15° C. for 12 h under N₂. The reaction mixture was quenched with saturated aqueous NH₄Cl solution (100 mL). The mixture was extracted with EtOAc (80 mL×3). The combined organic layers were washed with brine (100 mL), dried over Na₂SO₄, and filtered. The filtrate was concentrated under reduced pressure. The residue was purified by flash chromatography (ISCO®; 80 g SepaFlash@ Silica Flash Column, gradient elution of 0-25% ethyl acetate/petroleum ether @ 100 mL/min) which furnished ethyl 4-[hydroxyl (phenyl)methyl]-1-[(4-methoxyphenyl)methyl]pyrazole-3-carboxylate.

### Step 2

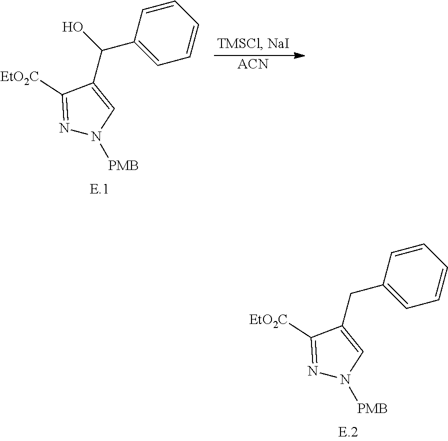

To a solution of NaI (4.66 g, 31.1 mmol, 6 eq) in MeCN (20 mL) was added TMSCl (3.38 g, 31.1 mmol, 3.95 mL, 6 eq) under N₂. After stirring at 15° C. for 10 minutes, a solution of ethyl 4-[hydroxyl (phenyl) methyl]-1-[(4-methoxyphenyl) methyl] pyrazole-3-carboxylate (1.9 g, 5.2 mmol, 1 eq) in MeCN (10 mL) was added. The mixture was stirred at 15° C. for 2 hours under N₂. The reaction mixture was quenched with saturated, aqueous Na₂SO₃ solution (150 mL). The mixture was extracted with EtOAc (80 mL×3). The combined organic layers were washed with brine (80 mL), dried over Na₂SO₄, and filtered. The filtrate was concentrated under reduced pressure. The residue was purified by gradient flash chromatography (ISCO®; 20 g SepaFlash@ Silica Flash Column, gradient elution of 0-20% ethyl acetate/petroleum ether @ 75 mL/min) which furnished ethyl 4-benzyl-1-[(4-methoxyphenyl)methyl]pyrazole-3-carboxylate.

### Step 3

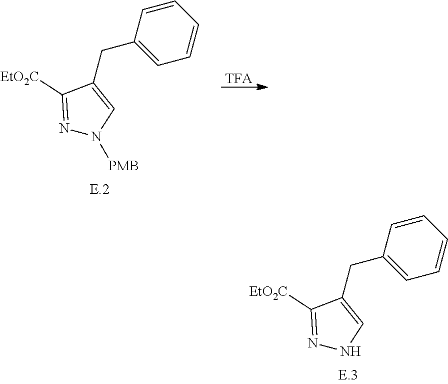

Ethyl 4-benzyl-1-[(4-methoxyphenyl)methyl]pyrazole-3-carboxylate (1.53 g, 4.37 mmol, 1 eq) was dissolved in TFA (20 mL). The mixture was stirred at 85° C. for 12 hr. The reaction mixture was concentrated under reduced pressure to remove TFA. The reaction mixture was diluted with H₂O (80 mL) and extracted with EtOAc (80 mL×3). The combined organic layers were washed with brine (60 mL), dried over Na₂SO₄, and filtered. The filtrate was concentrated under reduced pressure. The residue was purified by flash chromatography (ISCO®; 20 g SepaFlash® Silica Flash Column, gradient elution of 0 to 20% ethyl acetate/petroleum ether @ 100 mL/min) which furnished ethyl 4-benzyl-1H-pyrazole-3-carboxylate.

### Step 4

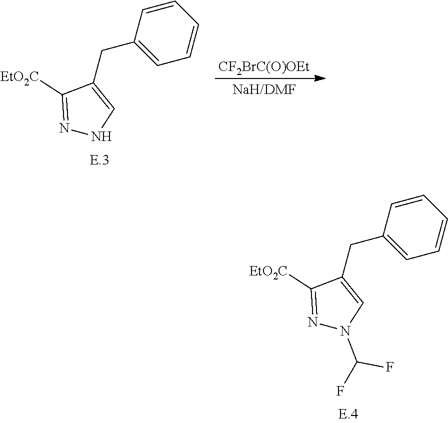 [SMILES: CCOC(=O)c1n[nH]cc1Cc1ccccc1 ; CCOC(=O)c1nn(C(F)F)cc1Cc1ccccc1 ; "CF2BrC(O)OEt"]

A mixture of ethyl 4-benzyl-1H-pyrazole-3-carboxylate (830 mg, 3.60 mmol, 1 eq) in DMF (10 mL) was cooled to 0° C. Sodium hydride (433 mg, 10.8 mmol, 60 wt % dispersion in oil, 3 eq) was added. After stirring for 20 min, ethyl 2-bromo-2,2-difluoro-acetate (878 mg, 4.33 mmol, 0.556 mL, 1.2 eq) was added dropwise at 0° C. The mixture was stirred at 15° C. for 2 h under N₂. The reaction mixture was quenched with saturated, aqueous NH₄C₁ (80 mL). The mixture was extracted with EtOAc (60 mL×3). The combined organic layers were washed with brine (50 mL), dried over Na₂SO₄, and filtered. The filtrate was concentrated under reduced pressure. The residue was purified by flash chromatography (ISCO®; 12 g SepaFlash@ Silica Flash Column, gradient elution of 0 to 3% ethyl acetate/petroleum ether @ 36 mL/min) which furnished 4-benzyl-1-(difluoromethyl) pyrazole-3-carboxylate.

### Example 18

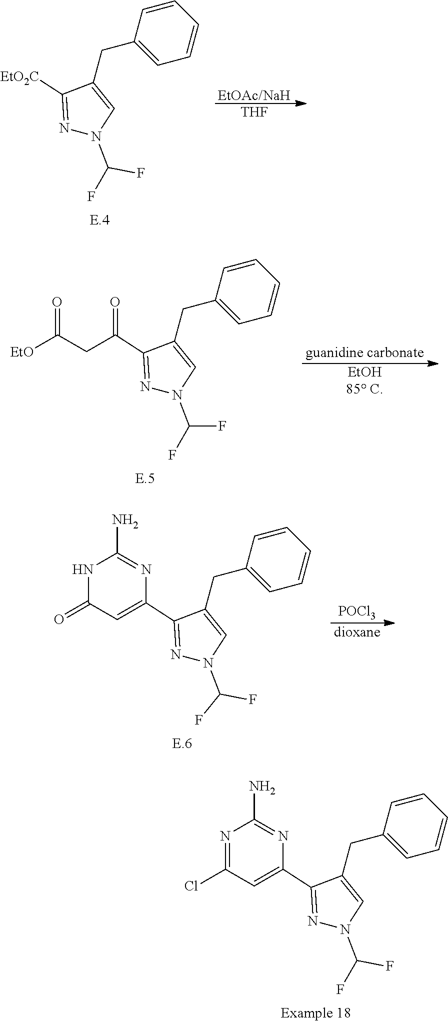

4-Benzyl-1-(difluoromethyl) pyrazole-3-carboxylate (E.4) was converted into Example 18 using conditions similar to that depicted in Steps 2-4 of Scheme A.

Example 18: ¹H NMR: (400 MHz, DMSO-d₆) δ 8.05 (s, 1H), 7.97-7.65 (m, 1H), 7.32-7.22 (m, 6H), 7.20-7.13 (m, 1H), 7.01 (s, 1H), 4.29 (s, 2H); LCMS: (MH+) 336.1.

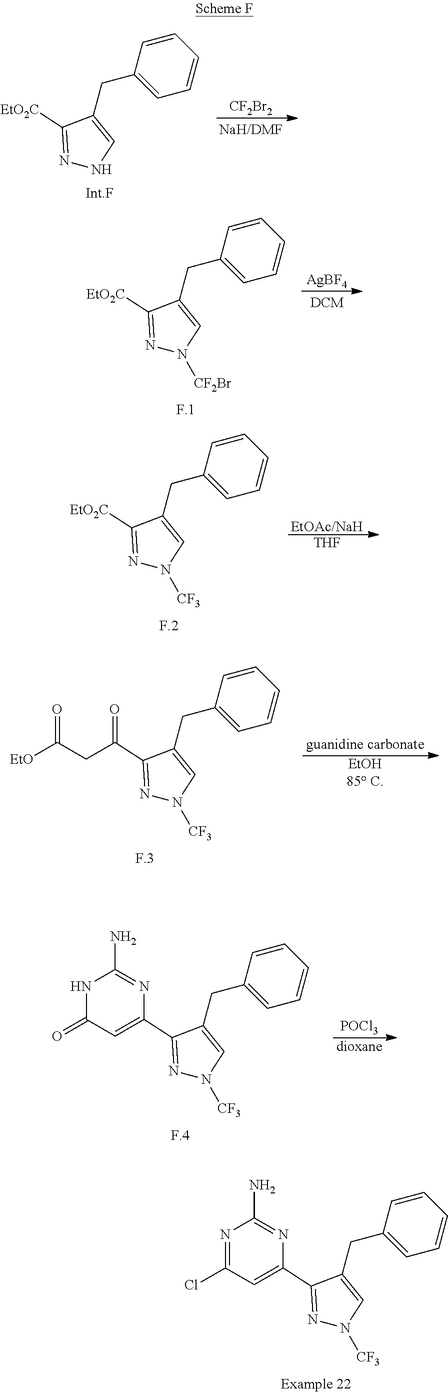

### Step 1

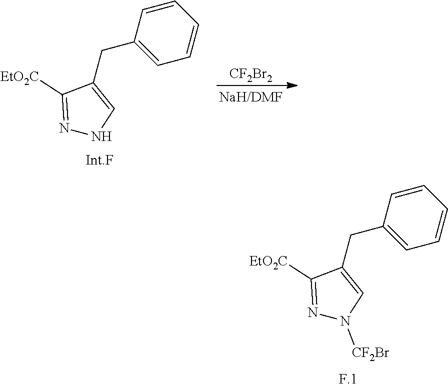

To a solution of ethyl 4-benzyl-1H-pyrazole-3-carboxylate (450 mg, 1.95 mmol, 1 eq) in DMF (5 mL) was added NaH (93.8 mg, 2.35 mmol, 60 wt % dispersion in oil, 1.2 eq) in portions at 0° C. The mixture was stirred at 0° C. for 30 min under N₂. A solution of dibromo(difluoro)methane (943 mg, 4.49 mmol, 0.415 mL, 2.3 eq) in DMF (5 mL) was added. The resulting mixture was stirred at 15° C. for 12 hr. The reaction mixture was quenched with saturated, aqueous NH₄Cl (60 mL). The mixture was extracted with EtOAc (50 mL×3). The combined organic layers were washed with brine (70 mL), dried over Na₂SO₄, and filtered. The filtrate was concentrated under reduced pressure. The residue was purified by flash chromatography (ISCO®; 20 g SepaFlash@ Silica Flash Column, gradient elution of 0-6% ethyl acetate/petroleum ether @ 70 mL/min) which furnished ethyl 4-benzyl-1-[bromo(difluoro)methyl]pyrazole-3-carboxylate.

### Step 2

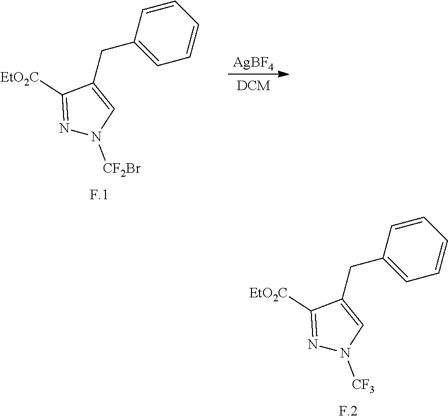

To a stirred solution of ethyl 4-benzyl-1-[bromo(difluoro)methyl]pyrazole-3-carboxylate (400 mg, 1.11 mmol, 1 eq) in DCM (6 mL) was added silver tetrafluoroborate (434 mg, 2.23 mmol, 2 eq) at −78° C. The reaction mixture was stirred at 15° C. for 12 h under N₂. The reaction mixture was diluted with DCM (20 mL) and filtered through a pad of Celite. The filter cake was washed with DCM (80 ml). The filtrate was concentrated under reduced pressure. The residue was purified by flash chromatography (ISCO®; 4 g SepaFlash@ Silica Flash Column, gradient elution of 0 to 6% ethyl acetate/petroleum ether @ 45 mL/min) which furnished ethyl 4-benzyl-1-(trifluoromethyl) pyrazole-3-carboxylate.

### Example 22

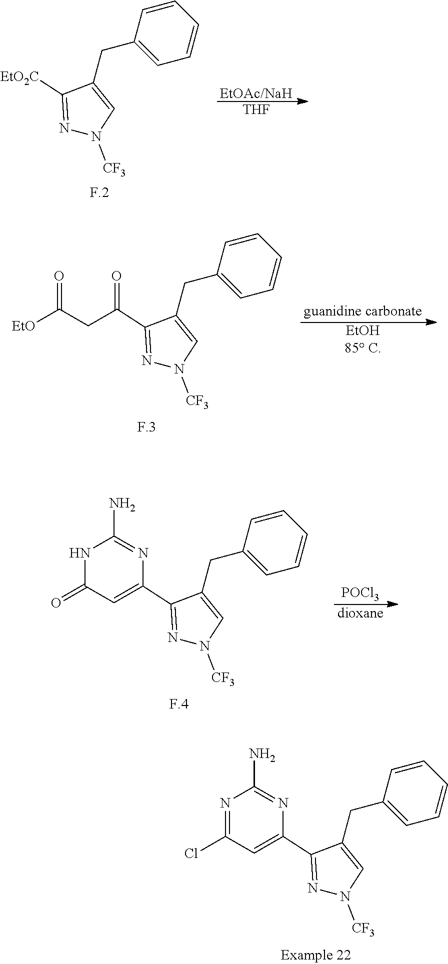

Ethyl 4-benzyl-1-(trifluoromethyl) pyrazole-3-carboxylate (F.2) was converted into Example 22 using conditions similar to that depicted in Scheme A (Steps 2-4).

Example 22: ¹H NMR: (400 MHz, DMSO-d₆) 8.41 (s, 1H), 7.35 (s, 2H), 7.30-7.21 (m, 4H), 7.18-7.12 (m, 1H), 7.00 (s, 1H), 4.30 (s, 2H); LCMS: (MH+) 354.0.

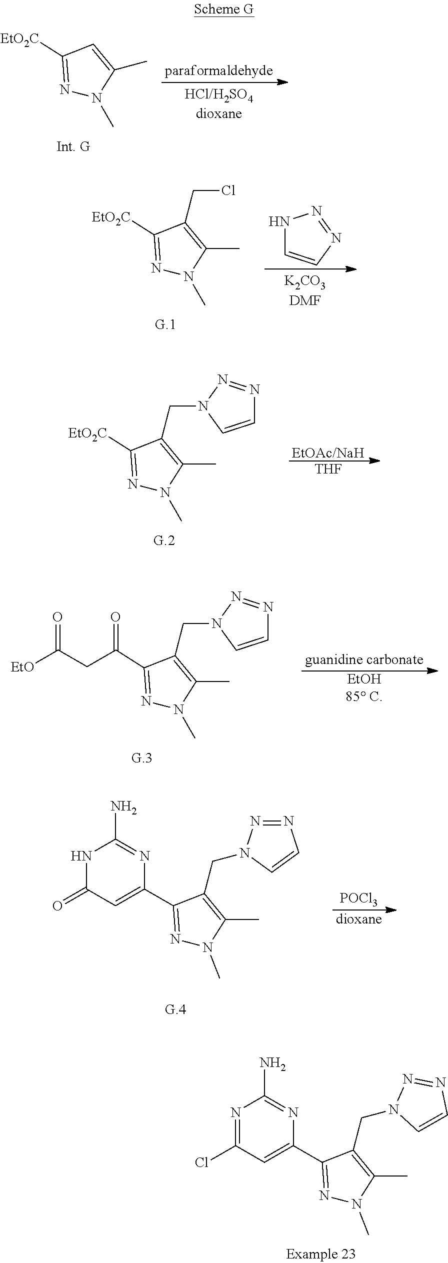

### Step 1

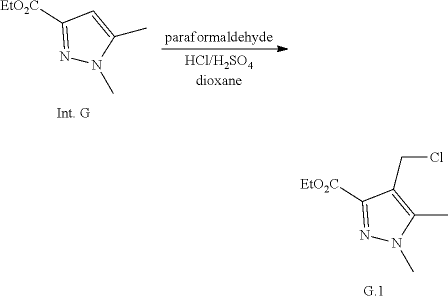

To a solution of ethyl 1,5-dimethylpyrazole-3-carboxylate (3.5 g, 20.8 mmol, 1 eq) and paraformaldehyde (1.25 g, 41.6 mmol, 2 eq) in dioxane (50 mL) was added aqueous HCl (12 M, 3.5 mL) and H₂SO₄ (208 mg, 2.08 mmol, 113. uL, 98% purity). The mixture was stirred at 100° C. for 2 hr. The reaction mixture was concentrated under reduced pressure which furnished ethyl 4-(chloromethyl)-1,5-dimethyl-pyrazole-3-carboxylate.

### Step 2

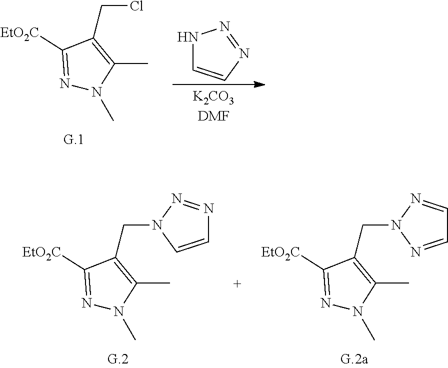

To a solution of ethyl 4-(chloromethyl)-1,5-dimethyl-pyrazole-3-carboxylate (2.00 g, 9.23 mmol, 1 eq) in DMF (20 mL) was added 1H-triazole (701 mg, 10.2 mmol, 0.589 mL, 1.1 eq) and K₂CO₃ (3.83 g, 27.7 mmol, 3 eq). The mixture was stirred at 50° C. for 3 hr under N₂. The reaction mixture was filtered through Celite. The filter cake was washed with EtOH (100 mL). The filtrate was concentrated under reduced pressure. The residue was purified by reverse-phase HPLC (neutral condition, MeCN/H₂O) which furnished two fractions:

Fraction 1 (G.2): ethyl 1,5-dimethyl-4-(triazol-1-ylmethyl)pyrazole-3-carboxylate. ¹H NMR: (400 MHz, DMSO-d₆) δ 7.93 (d, J=0.6 Hz, 1H), 7.66 (d, J=0.6 Hz, 1H), 5.63 (s, 2H), 4.25 (q, J=7.1 Hz, 2H), 3.81 (s, 3H), 2.31 (s, 3H), 1.25 (t, J=7.1 Hz, 3H).

Fraction 2 (G.2a): ethyl 1,5-dimethyl-4-(triazol-2-ylmethyl)pyrazole-3-carboxylate. ¹H NMR: (400 MHz, DMSO-d₆) δ 7.70 (s, 2H), 5.70 (s, 2H), 4.21 (q, J=7.1 Hz, 2H), 3.81 (s, 3H), 2.27 (s, 3H), 1.24 (t, J=7.1 Hz, 3H).

### Example 23

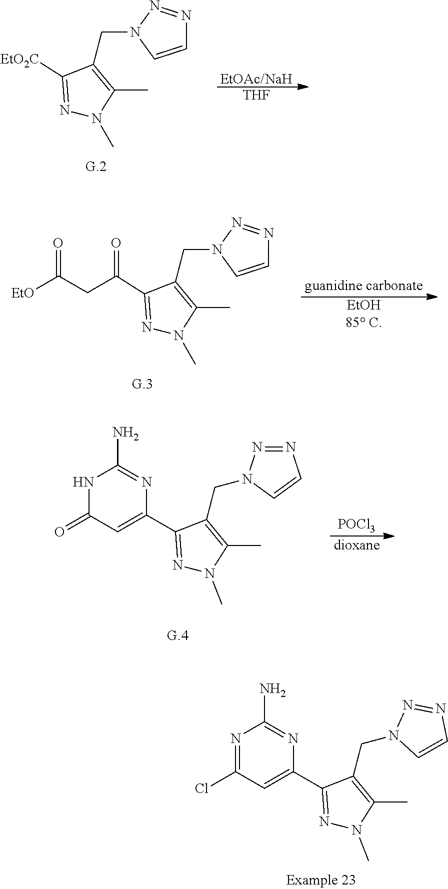

Example 23 was prepared from intermediate G.2 using conditions similar to that depicted in Scheme A (Steps 2-4).

Example 23: ¹H NMR: (400 MHz, DMSO-d₆) δ 8.11 (s, 1H), 7.62 (s, 1H), 7.25 (s, 2H), 7.01 (s, 1H), 5.91 (s, 2H), 3.81 (s, 3H), 2.36 (s, 3H); LCMS: (MH+) 305.1.

The following Examples in Table 3 were prepared in a similar fashion that that shown in Scheme G using the appropriate reagent/conditions for Step 2.

TABLE 3

| | | Reagent/ | | |
|---|---|---|---|---|
| Ex. | Structure | Conditions | 1H NMR (400 MHz) | LCMS |
| 24 | 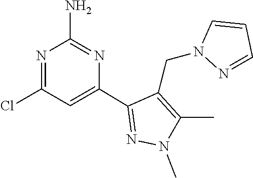 [SMILES: Cc1c(Cn2cccn2)c(-c2cc(Cl)nc(N)n2)nn1C] | 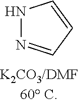 | (DMSO-d₆) δ 7.81 (s, 1H), 7.34 (s, 1H), 7.00 (d, J = 1.6 Hz, 1H), 6.12 (br d, J = 1.8 Hz, 1H), 5.65 (s, 2H), 3.79 (br d, J = 1.3 Hz, 3H), 2.33 (s, 3H) | 304.1 |
| 29 | 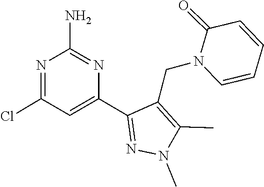 [SMILES: Cc1c(Cn2ccccc2=O)c(-c2cc(Cl)nc(N)n2)nn1C] | 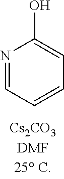 | (DMSO-d6) δ 7.70 (dd, J = 1.9, 6.9 Hz, 1H), 7.32 (ddd, J = 2.1, 6.7, 9.0 Hz, 1H), 7.13 (br s, 2H), 7.01 (s, 1H), 6.39-6.33 (m, 1H), 6.11 (dt, J = 1.4, 6.7 Hz, 1H), 5.45 (s, 2H), 3.79 (s, 3H), 2.29 (s, 3H) | 331.1 |

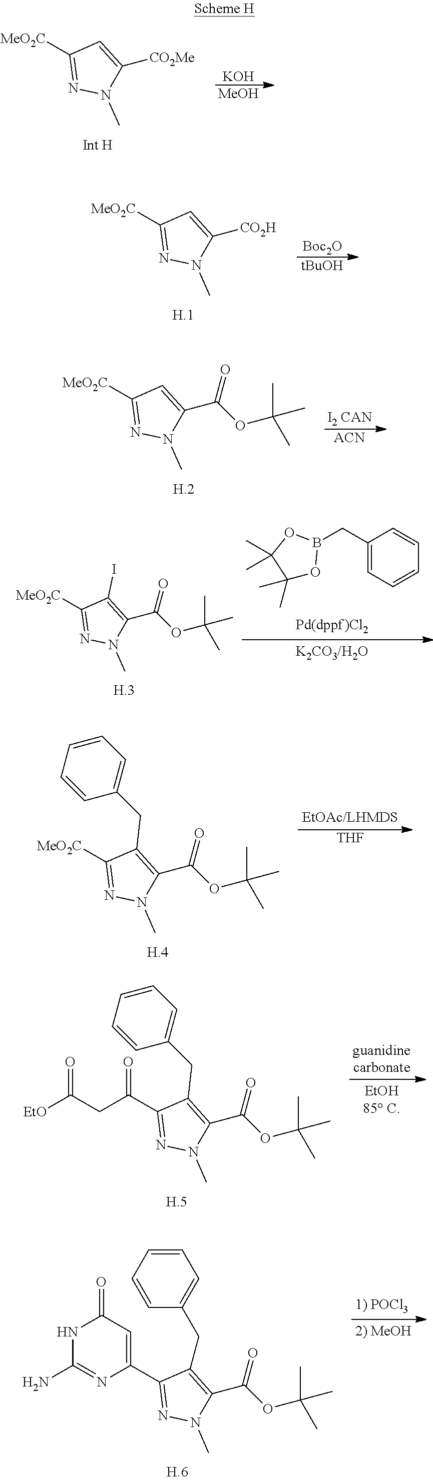

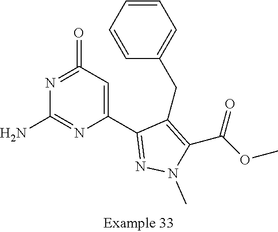

### Step 1

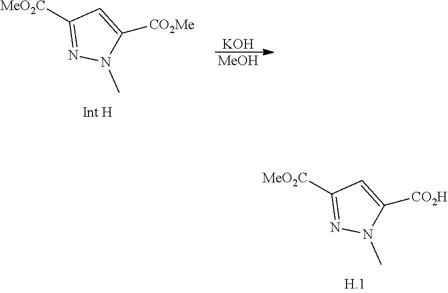

Dimethyl 1-methylpyrazole-3,5-dicarboxylate (5.60 g, 28.3 mmol, 1 eq) was dissolved in MeOH (56 mL). An aqueous solution of KOH (2.2 M, 13 mL) was added. The mixture was stirred at 15° C. for 12 h. The reaction mixture was diluted with water (150 mL) and extracted with DCM (50 mL×3). The water layer was adjusted to pH=5 by addition of aqueous 2 N HCl. The mixture was extracted with EtOAc (70 mL×3). The combined organic layers were washed with brine (150 mL), dried over Na₂SO₄, and filtered. The filtrate was concentrated under reduced pressure which furnished 5-methoxycarbonyl-2-methyl-pyrazole-3-carboxylic acid.

### Step 2

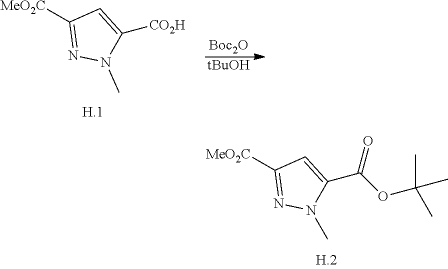 [SMILES: "Boc2O" ; "tBuOH" ; COC(=O)c1cc(C(=O)O)n(C)n1 ; COC(=O)c1cc(C(=O)OC(C)(C)C)n(C)n1]

To a suspension of 5-methoxycarbonyl-2-methyl-pyrazole-3-carboxylic acid (5.25 g, 28.5 mmol, 1 eq) and DMAP (697 mg, 5.70 mmol, 0.2 eq) in t-BuOH (100 mL) and THF (100 mL), Boc₂O (12.4 g, 57.0 mmol, 13.1 mL, 2 eq) was added at 15° C. The mixture was stirred at 15° C. for 12 h. The mixture was concentrated under reduced pressure. The residue was purified by column chromatography (SiO₂, petroleum ether/ethyl acetate=91/9) which furnished 5-(tert-butyl) 3-methyl 1-methyl-1H-pyrazole-3,5-dicarboxylate.

### Step 3

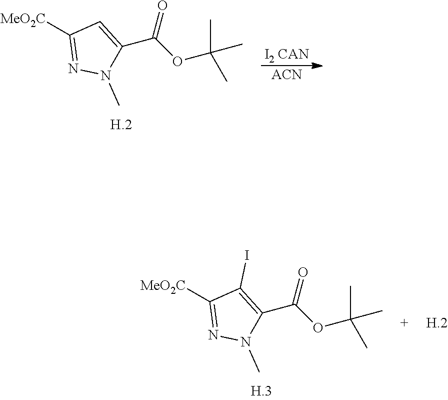

To a stirred mixture of 5-(tert-butyl) 3-methyl 1-methyl-1H-pyrazole-3,5-dicarboxylate (5.00 g, 20.8 mmol, 1 eq) in MeCN (100 mL) was added I₂ (3.17 g, 12.5 mmol, 2.52 mL, 0.6 eq) at 15° C. After stirring at 15° C. for 10 min, CAN (6.85 g, 12.5 mmol, 6.22 mL, 0.6 eq) was added in one portion. After the addition, the mixture was heated for 12 h at 80° C. The reaction mixture was diluted with water (70 mL) and extracted with EtOAc (40 mL×3). The combined organic layers were washed with brine (80 mL), dried over Na₂SO₄, and filtered. The filtrate was concentrated under the reduced pressure to afford the crude t-Bu ester H.3. The water layer was adjusted to pH=4 by addition of aqueous 1N HCl. The mixture extracted with EtOAc (45 mL×3). The combined organic layers were washed with brine (90 mL), dried over Na₂SO₄, and filtered. The filtrate was concentrated under the reduced pressure which furnished the crude acid H.2. The crude product H.3 was purified by column chromatography (SiO₂, petroleum ether/ethyl acetate=9/1). The crude product H.3 was used directly in next step without purification.

### Step 4

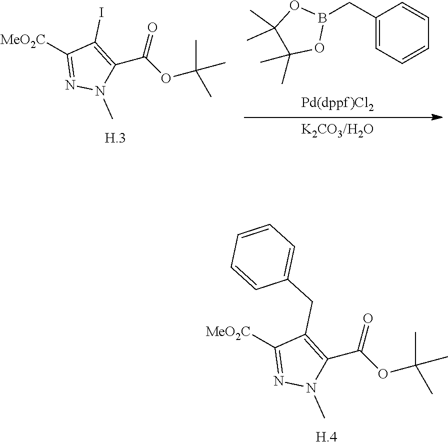

To a solution of H.3 (4.40 g, 13.3 mmol, 1 eq) and EtOAc (8.21 g, 93.2 mmol, 9.13 mL, 7 eq) in THF (80 mL) was added LiHMDS (1 M, 40.0 mL, 3 eq) at −40° C. in one portion. The mixture was stirred at −40° C. for 1 h under N₂. The reaction mixture was diluted with sat. aqueous NH₄Cl solution (150 mL) and extracted with EtOAc (100 mL×3). The organic layer was washed with brine (150 mL), dried over Na₂SO₄ and filtered. The filtrate was concentrated under reduced pressure. The residue was purified by column chromatography (SiO₂, petroleum ether/ethyl acetate=19/1) which furnished tert-butyl 4-benzyl-5-(3-ethoxy-3-oxo-propanoyl)-2-methyl-pyrazole-3-carboxylate.

### Example 33

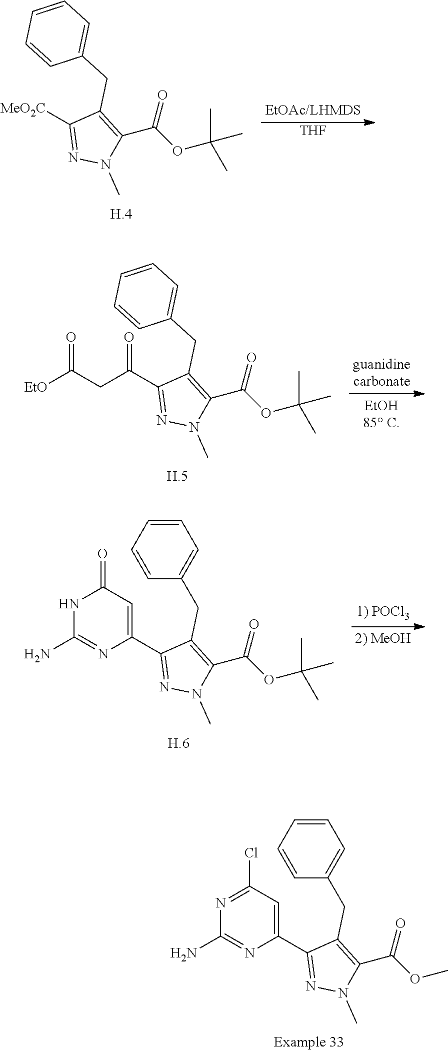 [SMILES: COC(=O)c1nn(C)c(C(=O)OC(C)(C)C)c1Cc1ccccc1 ; CCOC(=O)CC(=O)c1nn(C)c(C(=O)OC(C)(C)C)c1Cc1ccccc1 ; Cn1nc(-c2cc(=O)[nH]c(N)n2)c(Cc2ccccc2)c1C(=O)OC(C)(C)C ; "THF" ; COC(=O)c1c(Cc2ccccc2)c(-c2cc(Cl)nc(N)n2)nn1C]

H.4 was converted into Example 33 using conditions similar to those outlined in Steps 3-5 of Scheme C. The residue after treatment with POCl₃ was treated with MeOH and concentrated three times.

The residue was purified by column chromatography (SiO₂, petroleum ether/ethyl acetate=22/3) which afforded crude Example 33. The residue was further purified by neutral pre-HPLC (column: Welch Xtimate C18 150×25 mm, 5 m; mobile phase: [water (10 mM NH₄HCO₃)-ACN]; B %:50%-70%, 10 min) which afforded Example 33.

Example 33: ¹H NMR: (CDCl3, 400 MHz) δ 7.21-7.12 (m, 6H), 7.11-7.05 (m, 1H), 7.02 (s, 1H), 4.65 (s, 2H), 4.12 (s, 3H), 3.84 (s, 3H); LCMS: (MH+) 358.1.

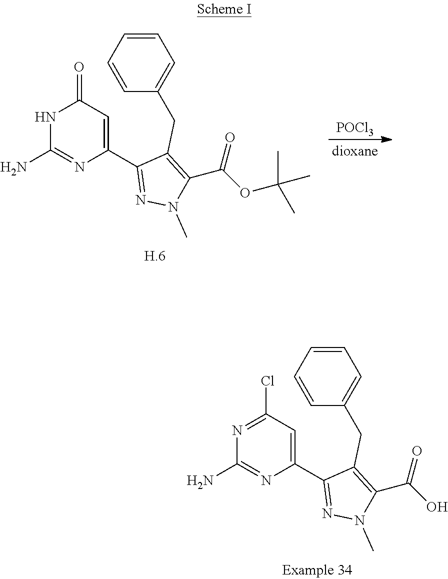 [SMILES: Cn1nc(-c2cc(=O)[nH]c(N)n2)c(Cc2ccccc2)c1C(=O)OC(C)(C)C ; "POCl3" ; Cn1nc(-c2cc(Cl)nc(N)n2)c(Cc2ccccc2)c1C(=O)O]

POCl₃ (398 mg, 2.60 mmol, 0.241 mL, 15 eq) was added to a solution of tert-butyl 5-(2-amino-6-oxo-1H-pyrimidin-4-yl)-4-benzyl-2-methyl-pyrazole-3-carboxylate (66.0 mg, 0.173 mmol, 1 eq) in dioxane (2 mL) at 15° C. Then the mixture was stirred for 12 h at 75° C. The reaction mixture was added slowly to aq. NaHCO₃ (saturated, 80 mL) to quench the excess POCl₃. The solution was extracted with EtOAc (30 mL×3). The water layer was adjusted to pH=4 by addition of aqueous HCl (1N). The water layer was extracted with EtOAc (40 mL×3). The combined organic layers were washed with brine (100 mL), dried over Na₂SO₄ and filtered. The filtrate was concentrated under reduced pressure. The residue was purified by neutral preparative-HPLC (column: Xtimate C18 150×25 mm, 5 m; mobile phase: [water (10 mM NH₄HCO₃)-ACN]; B %: 10%-40%, 8 min) which furnished Example 34.

Example 34: ¹H NMR: (DMSO-d6, 400 MHz) δ 7.23-7.20 (m, 2H), 7.14 (t, J=7.6 Hz, 2H), 7.09 (br s, 2H), 7.07-7.02 (m, 1H), 6.97 (s, 1H), 4.66 (s, 2H), 4.10 (s, 3H); LCMS: (MH+) 344.1.

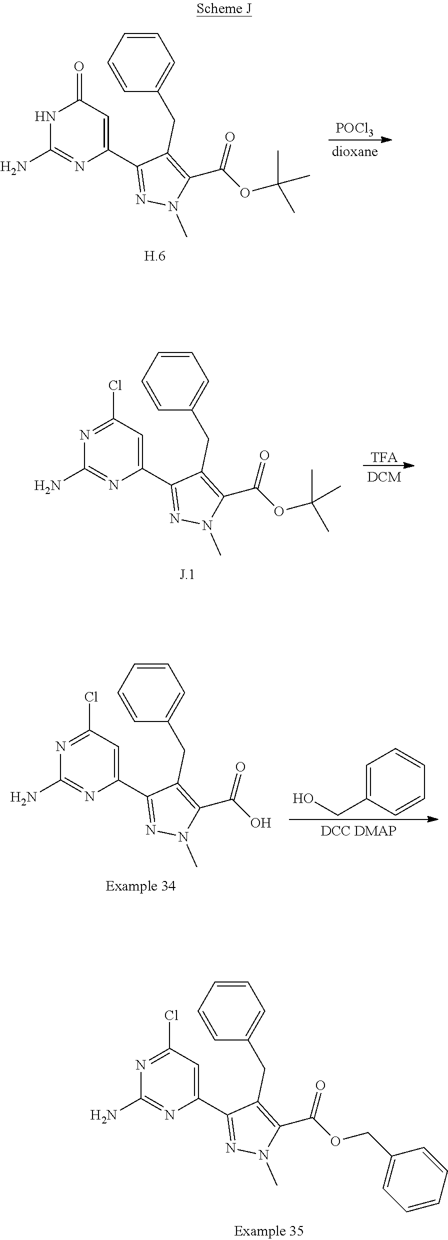

### Step 1

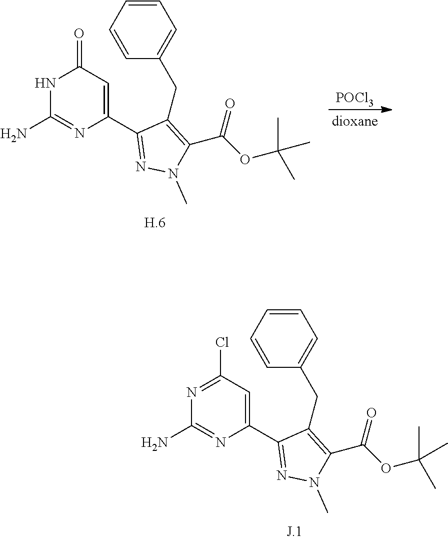 [SMILES: Cn1nc(-c2cc(=O)[nH]c(N)n2)c(Cc2ccccc2)c1C(=O)OC(C)(C)C ; "POCl3" ; Cn1nc(-c2cc(Cl)nc(N)n2)c(Cc2ccccc2)c1C(=O)OC(C)(C)C]

POCl₃ (13.9 g, 90.5 mmol, 8.41 mL, 15 eq) was added to a solution of tert-butyl 5-(2-amino-6-oxo-1H-pyrimidin-4-yl)-4-benzyl-2-methyl-pyrazole-3-carboxylate (2.3 g, 6.0 mmol, 1 eq) in dioxane (40 mL) at 15° C. The mixture was heated for 1.5 hr at 75° C. The reaction mixture was added slowly to aq. NaOH (1 N) to quench excess POC₃ (pH=8). The solution was extracted with EtOAc (100 mL×3). The organic layer was washed with brine (200 mL), dried over Na₂SO₄ and filtered. The filtrate was concentrated under reduced pressure. The residue was purified by column chromatography (SiO₂, petroleum ether/ethyl acetate=9/1) which furnished J.1.

### Step 2

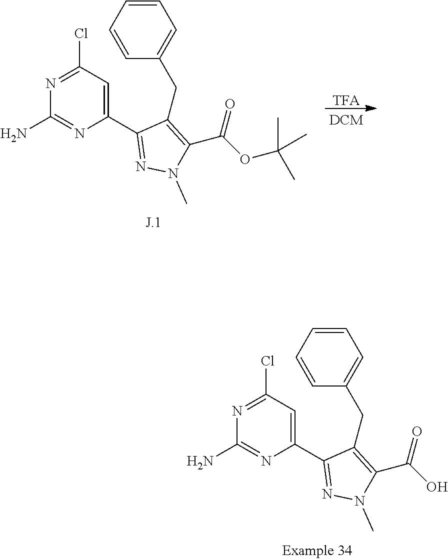 [SMILES: Cn1nc(-c2cc(Cl)nc(N)n2)c(Cc2ccccc2)c1C(=O)OC(C)(C)C ; "TFA" ; Cn1nc(-c2cc(Cl)nc(N)n2)c(Cc2ccccc2)c1C(=O)O]

A mixture of tert-butyl 5-(2-amino-6-chloro-pyrimidin-4-yl)-4-benzyl-2-methyl-pyrazole-3-carboxylate (200 mg, 0.500 mmol, 1 eq) in TFA (1 mL) and DCM (1 mL) was stirred at 15° C. for 2 h. The reaction mixture was diluted with water (40 mL). The solution was extracted with EtOAc (30 mL×3). The combined organic layer was washed with brine (50 mL), dried over Na₂SO₄, and filtered. The filtrate was concentrated under the reduced pressure. The residue was purified by column chromatography (SiO₂, petroleum ether/2-dimethyltetrahydrofuran, 3/2) which furnished 5-(2-amino-6-chloro-pyrimidin-4-yl)-4-benzyl-2-methyl-pyrazole-3-carboxylic acid Example 34.

### Step 3

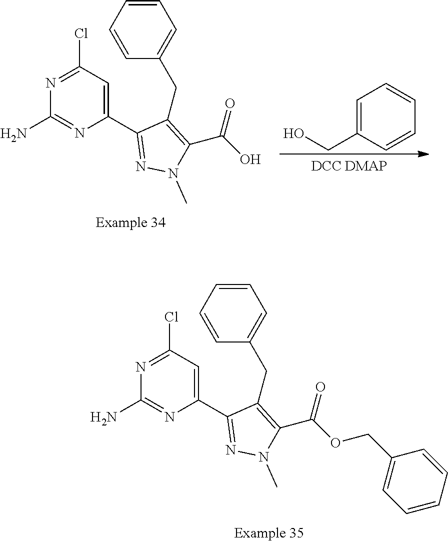 [SMILES: Cn1nc(-c2cc(Cl)nc(N)n2)c(Cc2ccccc2)c1C(=O)O ; OCc1ccccc1 ; Cn1nc(-c2cc(Cl)nc(N)n2)c(Cc2ccccc2)c1C(=O)OCc1ccccc1]

To a DCM (2 mL) solution of 5-(2-amino-6-chloro-pyrimidin-4-yl)-4-benzyl-2-methyl-pyrazole-3-carboxylic acid (70 mg, 0.20 mmol, 1 eq) were added phenylmethanol (44.0 mg, 0.407 mmol, 2 eq) and DCC (50.4 mg, 0.244 mmol, 1.2 eq), DMAP (6.2 mg, 0.051 mmol, 0.25 eq) at 0° C. The mixture was stirred at 15° C. for 12 h. The reaction mixture was diluted with water (40 mL). The solution was extracted with EtOAc (30 mL×3). The combined organic layer was washed with brine (50 mL), dried over Na₂SO₄, and filtered. The filtrate was concentrated under the reduced pressure. The residue was purified by neutral preparative-HPLC (column: Welch Xtimate C18 150×30 mm, 5 m; mobile phase: [water (10 mM NH₄HCO₃)-ACN]; B %: 55%-80%, 8 min) which furnished benzyl 5-(2-amino-6-chloro-pyrimidin-4-yl)-4-benzyl-2-methyl-pyrazole-3-carboxylate Example 35.

Example 35: ¹H NMR: (DMSO-d6, 400 MHz) 7.39-7.33 (m, 5H), 7.19-7.13 (m, 2H), 7.13-7.09 (m, 2H), 7.09-7.05 (m, 1H), 7.05-7.00 (i, 3H), 5.34 (s, 2H), 4.64 (s, 2H), 4.13 (s, 3H); LCMS: (MH+) 434.1

The following examples in Table 4 were prepared in a similar fashion to that depicted for Example 35 using the appropriate reagents for Step 3 of Scheme J.

TABLE 4

| | | Reagent/ | 1H NMR (400 | |
|---|---|---|---|---|
| Ex. | Structure | Conditions | MHz) | LCMS |
| 36 | 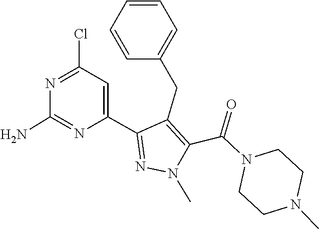 [SMILES: CN1CCN(C(=O)c2c(Cc3ccccc3)c(-c3cc(Cl)nc(N)n3)nn2C)CC1] | 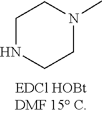 | (CD₃OD) δ 7.24-7.09 (m, 6H), 4.58 (br d, J = 15.3 Hz, 1H), 4.19 (br d, J = 15.3 Hz, 1H), 3.84 (s, 3H), 3.72-3.54 (m, 2H), 3.26- 3.17 (m, 1H), 2.96-2.87 (m, 1H), 2.48-2.32 (m, 2H), 2.22 (s, 3H), 2.20- 2.12 (m, 1H), 1.87 (br s, 1H) | 426.2 |
| 37 | 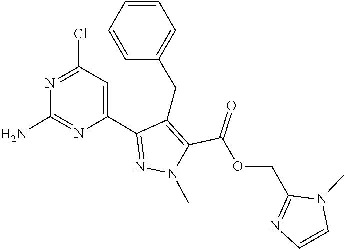 [SMILES: Cn1ccnc1COC(=O)c1c(Cc2ccccc2)c(-c2cc(Cl)nc(N)n2)nn1C] | 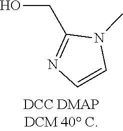 | (DMSO-d6) δ 7.21 (s, 1H), 7.16 (br s, 2H), 7.12-7.04 (m, 3H), 7.02 (s, 1H), 6.93 (t, J = 3.4 Hz, 3H), 5.36 (s, 2H), 4.58 (s, 2H), 4.12 (s, 3H), 3.52 (s, 3H) | 438.2 |
| 38 | 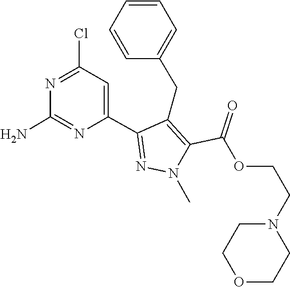 [SMILES: Cn1nc(-c2cc(Cl)nc(N)n2)c(Cc2ccccc2)c1C(=O)OCCN1CCOCC1] | 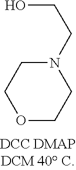 | (DMSO-d6) δ 7.20-7.12 (m, 6H), 7.10-7.04 (m, 1H), 7.00 (s, 1H), 4.69 (s, 2H), 4.38 (t, J = 5.4 Hz, 2H), 4.14 (s, 3H), 3.50-3.43 (m, 4H), 2.58 (br t, J = 5.4 Hz, 2H), 2.35 (br s, 4H) | 457.2 |
| 39 | 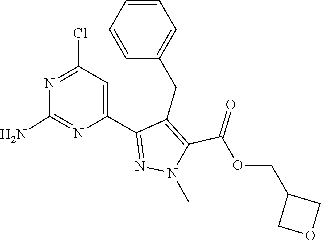 [SMILES: Cn1nc(-c2cc(Cl)nc(N)n2)c(Cc2ccccc2)c1C(=O)OCC1COC1] | 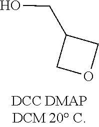 | (DMSO-d6) δ 7.22-7.05 (m, 7H), 7.00 (s, 1H), 4.66 (s, 2H), 4.57 (t, J = 7.0 Hz, 2H), 4.47 (d, J = 6.5 Hz, 2H), 4.31 (t, J = 6.1 Hz, 2H), 4.15 (s, 3H), 3.30-3.22 (m, 1H) | 414.1 |
| 40 | 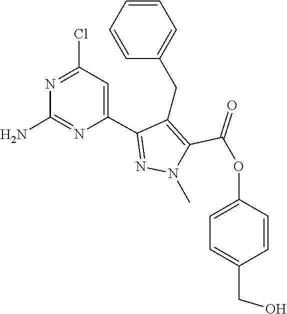 [SMILES: Cn1nc(-c2cc(Cl)nc(N)n2)c(Cc2ccccc2)c1C(=O)Oc1ccc(CO)cc1] | 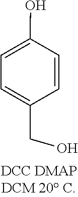 | (DMSO-d6) δ 7.37 (d, J = 8.4 Hz, 2H), 7.23-7.15 (m, 6H), 7.14-7.05 (m, 4H), 5.25 (t, J = 5.7 Hz, 1H), 4.78 (s, 2H), 4.51 (d, J = 5.6 Hz, 2H), 4.20 (s, 3H) | 450.1 |
| 41 | 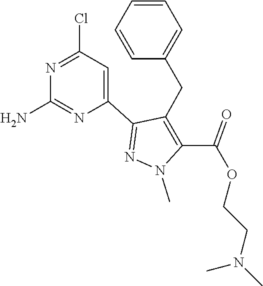 [SMILES: CN(C)CCOC(=O)c1c(Cc2ccccc2)c(-c2cc(Cl)nc(N)n2)nn1C] | 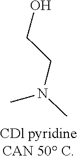 | (CDCl₃) δ 7.27-7.26 (m, 1H), 7.25-7.20 (m, 2H), 7.17- 7.11 (m, 3H), 5.04 (br s, 2H), 4.63 (s, 2H), 4.37 (t, J = 5.7 Hz, 2H), 4.23 (s, 3H), 2.60 (t, J = 6.1 Hz, 2H), 2.26 (s, 6H) | 415.1 |
| 42 | 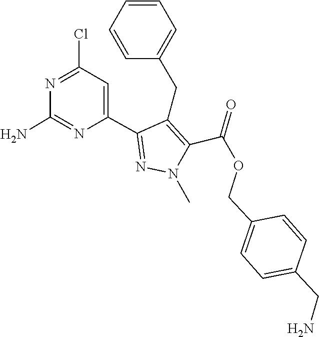 [SMILES: Cn1nc(-c2cc(Cl)nc(N)n2)c(Cc2ccccc2)c1C(=O)OCc1ccc(CN)cc1] | 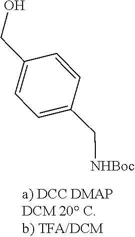 [SMILES: CC(C)(C)OC(=O)NCc1ccc(CO)cc1] | (DMSO-d₆) δ 7.31 (s, 4H), 7.18-7.10 (m, 4H), 7.09-7.02 (m, 3H), 7.01 (s, 1H), 5.31 (s, 2H), 4.64 (s, 2H), 4.12 (s, 3H), 3.71 (s, 2H) | 463.2 |
| 65 | 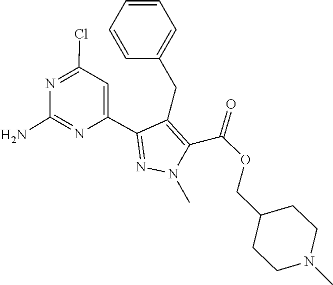 [SMILES: CN1CCC(COC(=O)c2c(Cc3ccccc3)c(-c3cc(Cl)nc(N)n3)nn2C)CC1] | 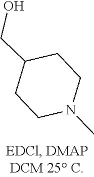 | (DMSO-d6) δ 7.21-7.05 (m, 7H), 7.01 (s, 1H), 4.68 (s, 2H), 4.14 (s, 3H), 4.12 (s, 2H), 2.69 (br d, J = 1.8 Hz, 2H), 2.12 (s, 3H), 1.76 (br t, J = 10.8 Hz, 2H), 1.60-1.48 (m, 3H), 1.18 (dq, J = 6, 11.9 Hz, 2H) | 455.1 |
| 66 | 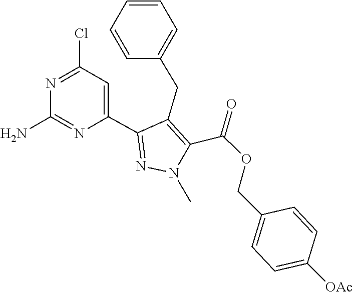 [SMILES: CC(=O)Oc1ccc(COC(=O)c2c(Cc3ccccc3)c(-c3cc(Cl)nc(N)n3)nn2C)cc1] | 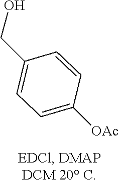 [SMILES: CC(=O)Oc1ccc(CO)cc1] | (DMSO-d6) δ 7.40 (d, J = 8.5 Hz, 2H), 7.18-7.09 (m, 6H), 7.08-7.04 (m, 1H), 7.04- 6.99 (m, 3H), 5.34 (s, 2H), 4.65 (s, 2H), 4.13 (s, 3H), 2.27 (s, 3H) | 492.1 |
| 67 | 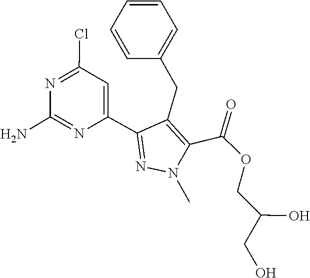 [SMILES: Cn1nc(-c2cc(Cl)nc(N)n2)c(Cc2ccccc2)c1C(=O)OCC(O)CO] | 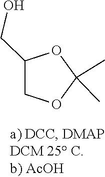 | (DMSO-d6) δ 7.22-7.12 (m, 6H), 7.10-7.04 (m, 1H), 7.01 (s, 1H), 5.05 (d, J = 5.1 Hz, 1H), 4.75-4.70 (m, 1H), 4.68 (s, 2H), 4.34 (dd, J = 3.8, 11.1 Hz, 1H), 4.19 (dd, J = 6.5, 11.1 Hz, 1H), 4.14 (s, 3H), 3.81-3.73 (m, 1H), 3.44- 3.34 (m, 2H) | 418.1 |
| 68 | 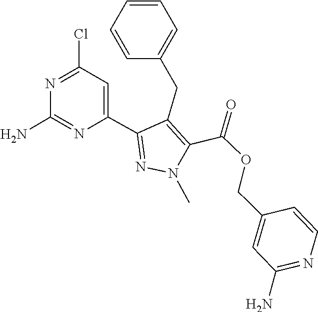 [SMILES: Cn1nc(-c2cc(Cl)nc(N)n2)c(Cc2ccccc2)c1C(=O)OCc1ccnc(N)c1] | 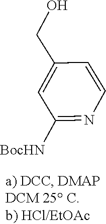 [SMILES: CC(C)(C)OC(=O)Nc1cc(CO)ccn1] | (DMSO-d₆) δ 7.84 (d, J = 5.1 Hz, 1H), 7.21-7.15 (m, 2H), 7.15-7.12 (m, 2H), 7.11- 7.04 (m, 3H), 7.03 (s, 1H), 6.42-6.38 (m, 2H), 5.96 (s, 2H), 5.20 (s, 2H), 4.68 (s, 2H), 4.15 (s, 3H) | 450.1 |
| 73 | 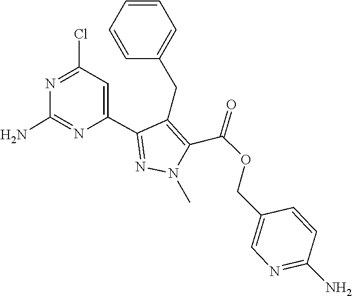 [SMILES: Cn1nc(-c2cc(Cl)nc(N)n2)c(Cc2ccccc2)c1C(=O)OCc1ccc(N)nc1] | 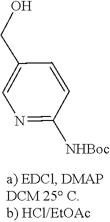 [SMILES: CC(C)(C)OC(=O)Nc1ccc(CO)cn1] | (DMSO-d₆, formic acid salt) δ 8.18 (s, 1H), 7.97 (d, J = 1.9 Hz, 1H), 7.37 (dd, J = 2.3, 8.5 Hz, 1H), 7.16 (br s, 2H), 7.13-7.02 (m, 5H), 7.01 (s, 1H), 6.40 (d, J = 8.5 Hz, 1H), 6.09 (s, 2H), 5.15 (s, 2H), 4.60 (s, 2H), 4.10 (s, 3H) | 450.0 |

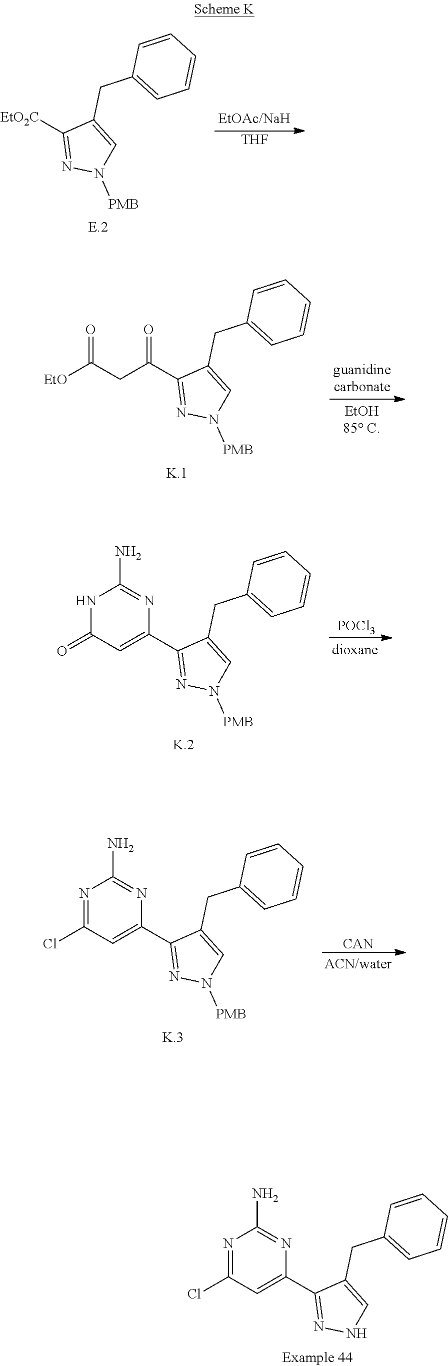

The intermediate E.2 was converted into K.3 using conditions similar to those outlined in Scheme E (E.4 to Example 18).

### Example 44

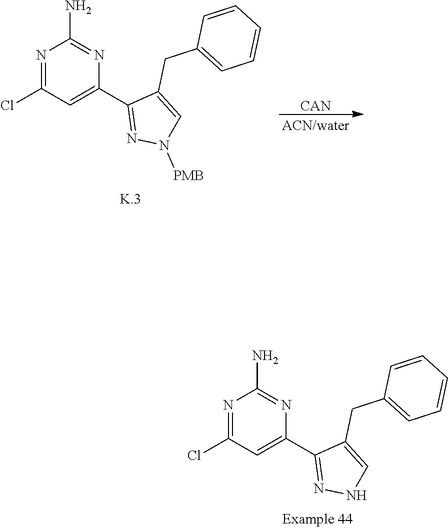

To a solution of 4-[4-benzyl-1-[(4-methoxyphenyl)methyl] pyrazol-3-yl]-6-chloro-pyrimidin-2-amine (30 mg, 0.074 mmol, 1 eq) in MeCN (1 mL) and H₂O (1 mL) was added CAN (122 mg, 0.222 mmol, 3 eq) at 0° C. The reaction mixture was stirred at 0° C. for 0.5 hr and then warmed to 20° C. The reaction mixture was stirred at 20° C. for 12 hr. The reaction mixture was quenched with saturated aqueous NaHCO₃ solution (5 mL), diluted with H₂O (20 mL) and extracted with EtOAc (30 mL×3). The combined organic layers were washed with brine (60 mL), dried over Na₂SO₄, and filtered. The filtrate was concentrated under reduced pressure. The residue was purified by preparative-HPLC (column: Waters Xbridge 150×25 mm, 5 m; mobile phase: [water (10 mM NH₄HCO₃)-ACN]; B %: 35%-55%, 10 min) which furnished 4-(4-benzyl-1H-pyrazol-3-yl)-6-chloro-pyrimidin-2-amine Example 44.

Example 44: ¹H NMR: (400 MHz, DMSO-d₆) δ 7.55 (s, 1H), 7.31-7.18 (m, 4H), 7.16-7.06 (m, 3H), 7.04 (s, 1H), 4.28 (s, 2H); LCMS: (MH+) 286.1.

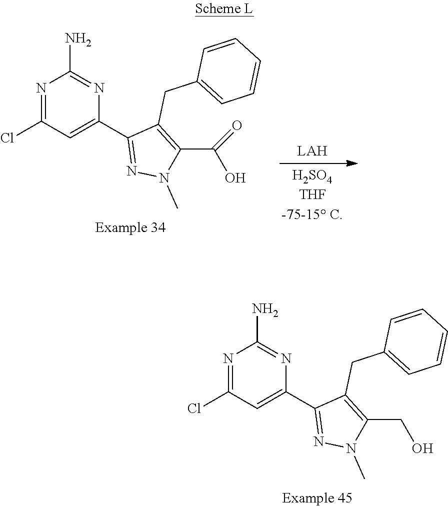 [SMILES: Cn1nc(-c2cc(Cl)nc(N)n2)c(Cc2ccccc2)c1C(=O)O ; Cn1nc(-c2cc(Cl)nc(N)n2)c(Cc2ccccc2)c1CO]

To a mixture of LiAlH₄ (760 mg, 20 mmol, 34 eq) in THF (20 mL) was added H₂SO₄ (0.6 mL) at −78° C. dropwise. The mixture was stirred at −78° C. for 2 h and then at 15° C. for 2 hours (white solid appeared). The freshly prepared alane solution (7 mL) was cooled to 0° C., and 5-(2-amino-6-chloro-pyrimidin-4-yl)-4-benzyl-2-methyl-pyrazole-3-carboxylic acid (200 mg, 0.582 mmol, 1 eq) in THF (5 mL) was added at 0° C. The mixture was stirred at 15° C. for 0.5 h. The reaction mixture was diluted with water (40 mL). The solution was extracted with EtOAc (30 mL×6). The combined organic layer was washed with brine (50 mL), dried over Na₂SO₄, and filtered. The filtrate was concentrated under the reduced pressure. The residue was purified by column chromatography (SiO₂, petroleum ether/ethyl acetate=3/1) which provided the crude product. The crude product was further purified by neutral preparative-HPLC (column: Welch Xtimate C18 150*25 mm, 5 m; mobile phase: [water (10 mM NH₄HCO₃)-ACN]; B %: 30%-50%, 9 min) which furnished [5-(2-amino-6-chloro-pyrimidin-4-yl)-4-benzyl-2-methyl-pyrazol-3-yl]methanol Example 45.

Example 45: ¹H NMR: (CD3OD, 400 MHz) δ 7.20-7.15 (m, 4H), 7.11-7.05 (m, 2H), 4.60 (s, 2H), 4.37 (s, 2H), 3.97 (s, 3H); LCMS: (MH+) 330.1.

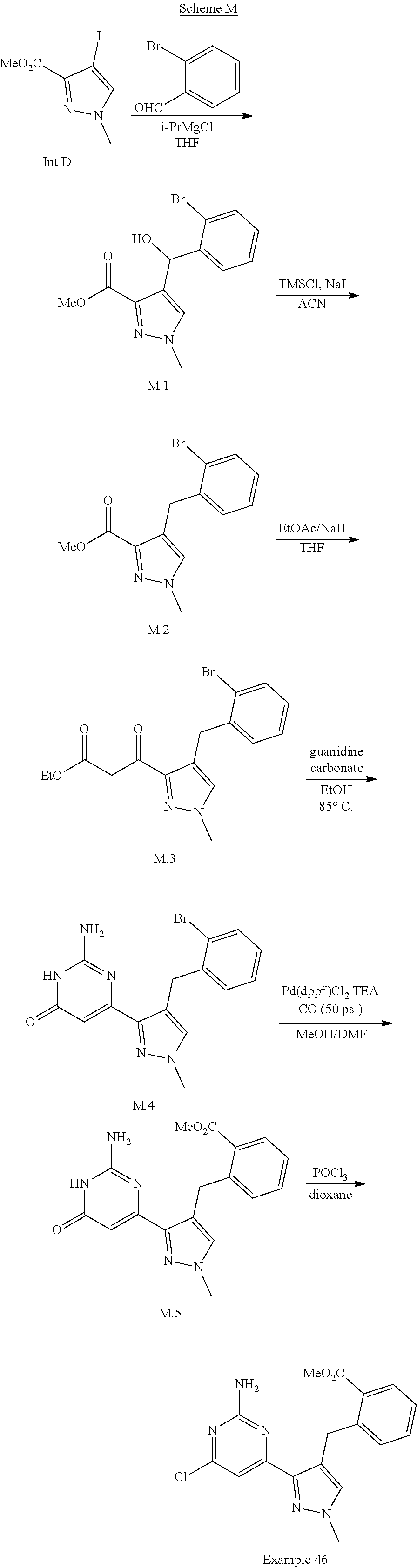

Intermediate M.4 was prepared from Int D using conditions similar to those outlined in Scheme B for B.4 from Int A.

### Step 5

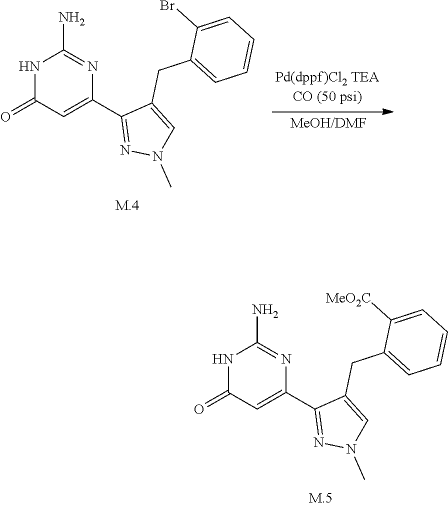 [SMILES: Cn1cc(Cc2ccccc2Br)c(-c2cc(=O)[nH]c(N)n2)n1 ; COC(=O)c1ccccc1Cc1cn(C)nc1-c1cc(=O)[nH]c(N)n1]

To a solution of 2-amino-4-[4-[(2-bromophenyl)methyl]-1-methyl-pyrazol-3-yl]-1H-pyrimidin-6-one (550 mg, 1.53 mmol, 1 eq) in MeOH (8 mL) and DMF (4 mL) was added TEA (618 mg, 6.11 mmol, 4 eq) and Pd(dppf)Cl₂ (112 mg, 0.152 mmol, 0.1 eq). The reaction mixture was degassed and purged with CO three times. The reaction was stirred at 80° C. for 48 hr under CO (50 psi). Additional Pd(dppf)Cl₂ (111.72 mg, 0.153 mmol, 0.1 eq) was added, and the mixture was degassed and purged with CO three times. Then the reaction was at 80° C. for 15 hr under CO (50 psi). The reaction was filtered through Celite. The filtrate was concentrated under reduced pressure. The residue was purified by reversed-phase HPLC (neutral condition, MeCN and H₂O) which provided methyl 2-[[3-(2-amino-6-oxo-1H-pyrimidin-4-yl)-1-methyl-pyrazol-4-yl]methyl]benzoate.

### Example 46

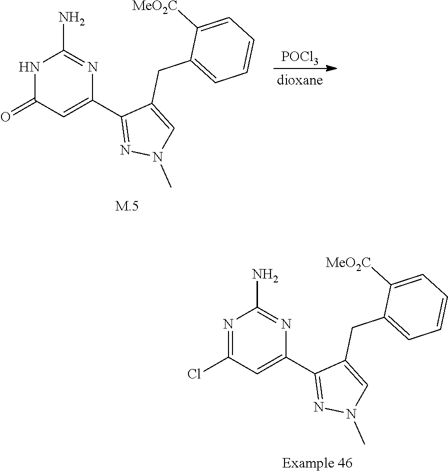

Example 46 was prepared from M.5 similar to that previously described in Scheme B for Example 2.

Example 46: ¹H NMR: (400 MHz, DMSO-d₆) δ 7.76 (dd, J=1.1, 7.8 Hz, 1H), 7.50-7.43 (m, 1H), 7.38-7.23 (m, 3H), 7.20 (s, 1H), 7.01 (s, 2H), 4.55 (s, 2H), 3.80 (s, 3H), 3.76 (s, 3H); LCMS: (MH+) 358.1.

The examples in Table 5 were prepare in a similar fashion to Example 46 in Scheme M using the appropriate aldehyde in Step 1.

TABLE 5

| | | | 1H NMR (400 | |
|---|---|---|---|---|
| Ex. | Structure | Aldehyde | MHz) | LCMS |
| 47 | 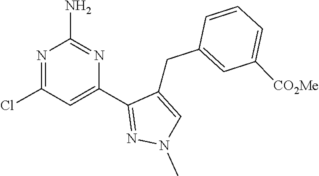 [SMILES: COC(=O)c1cccc(Cc2cn(C)nc2-c2cc(Cl)nc(N)n2)c1] | 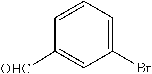 | (DMSO-d₆) δ7.84 (s, 1H), 7.74 (d, J = 7.6 Hz, 1H), 7.61-7.56 (m, 2H), 7.43-7.36 (m, 1H), 7.09 (br s, 2H), 6.98 (s, 1H), 4.34 (s, 2H), 3.85 (s, 3H), 3.81 (s, 3H) | 358.1 |

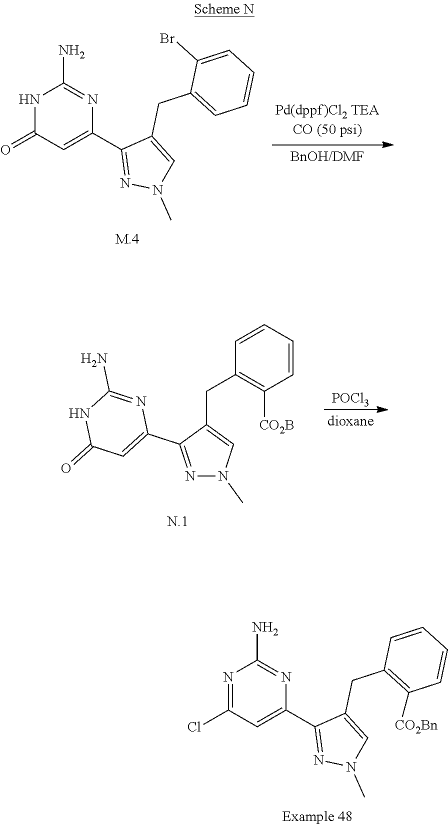

Example 48 was prepared from M.4 using benzyl alcohol in a similar fashion to that described in Scheme M.

Example 48: ¹H NMR: (400 MHz, DMSO-d₆) δ 7.81 (d, J=7.7 Hz, 1H), 7.5-7.44 (m, 1H), 7.40-7.30 (m, 7H), 7.10 (s, 1H), 6.99 (s, 1H), 5.25 (s, 2H), 4.56 (s, 2H), 3.76 (s, 3H); LCMS: (MH+) 434.1.

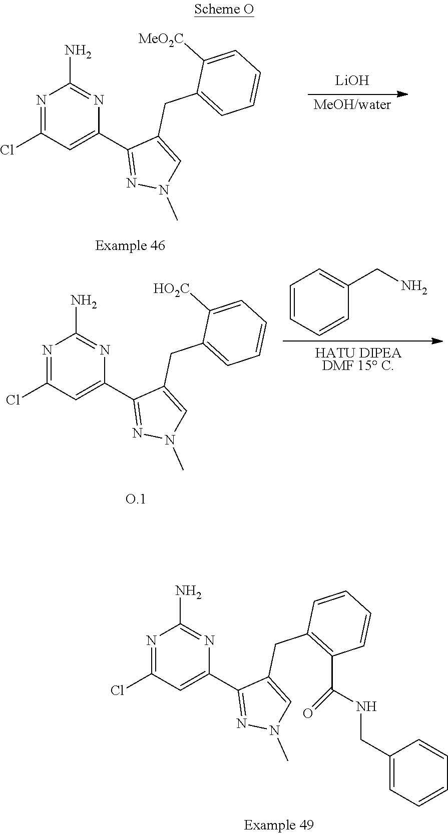

### Step 1

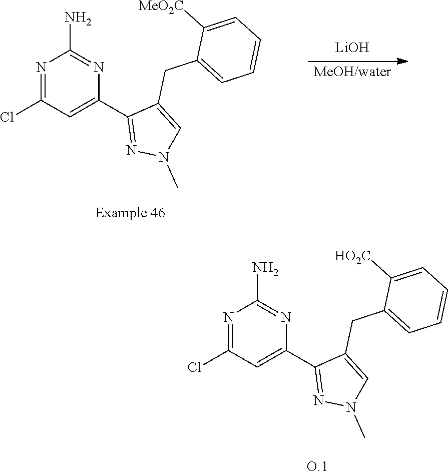 [SMILES: COC(=O)c1ccccc1Cc1cn(C)nc1-c1cc(Cl)nc(N)n1 ; "LiOH" ; "MeOH/water" ; Cn1cc(Cc2ccccc2C(=O)O)c(-c2cc(Cl)nc(N)n2)n1]

To a solution of methyl 2-[[3-(2-amino-6-chloro-pyrimidin-4-yl)-1-methyl-pyrazol-4-yl]methyl]benzoate (137 mg, 0.383 mmol, 1.0 eq) in H₂O (2 mL) and dioxane (5 mL) was added LiOH·H₂O (80 mg, 1.9 mmol, 5.0 eq). The mixture was stirred at 60° C. for 2 hr. The reaction was diluted with H₂O (10 mL) and extracted with DCM (10 mL×2). The aqueous layer was acidified by the addition of aqueous HCl (1M) to pH-6. The resulting mixture was extracted with DCM (30 mL×5). The organic layer was washed with brine (20 mL), dried over Na₂SO₄, and filtered. The filtrate was concentrated which furnished 2-[[3-(2-amino-6-chloro-pyrimidin-4-yl)-1-methyl-pyrazol-4-yl]methyl]benzoic acid.

### Step 2

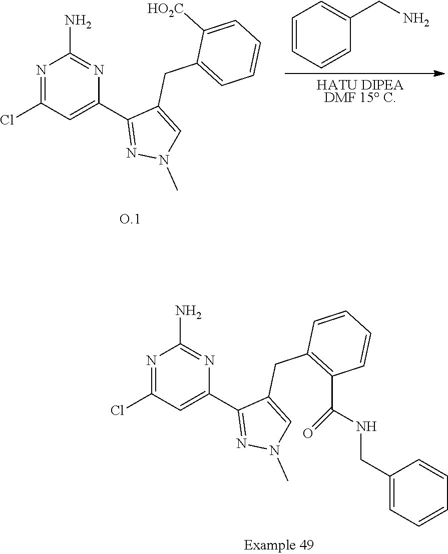

A mixture of 2-[[3-(2-amino-6-chloro-pyrimidin-4-yl)-1-methyl-pyrazol-4-yl]methyl] benzoic acid (200 mg, 0.582 mmol, 1 eq), phenylmethanamine (62 mg, 0.58 mmol, 1 eq), HATU (332 mg, 0.873 mmol, 1.5 eq), DIPEA (226 mg, 1.75 mmol, 3 eq) in DMF (3 mL) was degassed and purged with N₂ (3×). The mixture was stirred at 15° C. for 4 hr under N₂ atmosphere. The reaction was diluted with MeOH (1 mL). The solution was purified by preparative-HPLC (column: Phenomenex Luna C18 100×30 mm, 5 m; mobile phase: [water (0.2% FA)-ACN]; B %: 30%-60%, 10 min) which furnished 2-[[3-(2-amino-6-chloro-pyrimidin-4-yl)-1-methyl-pyrazol-4-yl]methyl]-N-benzyl-benzamide Example 49.

Example 49: ¹H NMR: (400 MHz, DMSO-d₆) δ 8.85 (br t, J=6.0 Hz, 1H), 7.38-7.34 (m, 2H), 7.32-7.13 (m, 8H), 7.08 (s, 2H), 6.98 (s, 1H), 4.43 (d, J=6.0 Hz, 2H), 4.34 (s, 2H), 3.79 (s, 3H); LCMS: (MH+) 433.2.

The examples in Table 6 were prepared in a similar fashion to that depicted in Scheme 0 using the appropriate reagents for Step 2.

TABLE 6

| | | | 1H NMR | |
|---|---|---|---|---|
| Ex. | Structure | Conditions | (400 MHz) | LCMS |
| 52 | 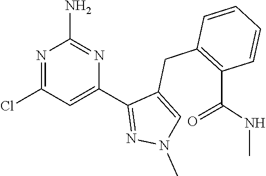 [SMILES: CNC(=O)c1ccccc1Cc1cn(C)nc1-c1cc(Cl)nc(N)n1] | MeNH₂-HCl HOBT, EDCI DIPEA, DMF 15° C. | (DMSO-d₆) δ 8.22 (br d, J = 4.6 Hz, 1H), 7.50 (s, 1H), 7.30-7.21 (m, 2H), 7.21-7.15 (m, 1H), 7.13-7.06 (m, 3H), 6.96 (s, 1H), 4.30 (s, 2H), 3.85 (s, 3H), 2.74 (d, J = 4.6 Hz, 3H) | 357.2 |
| 53 | 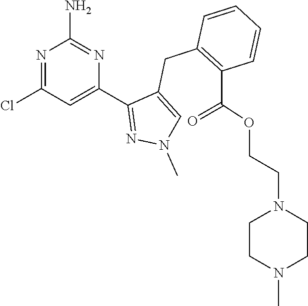 [SMILES: CN1CCN(CCOC(=O)c2ccccc2Cc2cn(C)nc2-c2cc(Cl)nc(N)n2)CC1] | 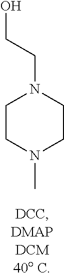 | (400 MHz) δ 7.79 (d, J = 7.5 Hz, 1H), 7.45-7.38 (m, 1H), 7.31-7.24 (m, 2H), 7.15 (s, 1H), 7.13 (s, 1H), 4.58 (s, 2H), 4.36 (t, J = 5.7 Hz, 2H), 3.86 (s, 3H), 2.65 (t, J = 5.7 Hz, 2H), 2.61- 2.30 (m, 8H), 2.22 (s, 3H) | 470.1 |
| 54 | 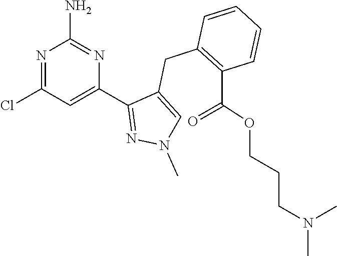 [SMILES: CN(C)CCCOC(=O)c1ccccc1Cc1cn(C)nc1-c1cc(Cl)nc(N)n1] | 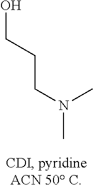 | (CD3OD) δ 7.81 (dd, J = 1.3, 8.1 Hz, 1H), 7.47-7.41 (m, 1H), 7.34-7.27 (m, 2H), 7.13 (s, 1H), 7.10 (s, 1H), 4.57 (s, 2H), 4.24 (t, J = 6.4 Hz, 2H), 3.85 (s, 3H), 2.41- 2.33 (m, 2H), 2.20 (s, 6H), 1.87-1.78 (m, 2H) | 429.2 |
| 55 | 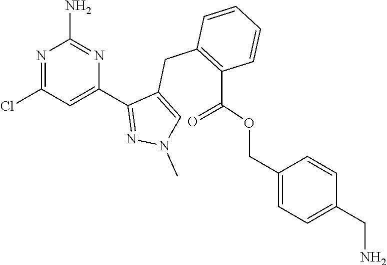 [SMILES: Cn1cc(Cc2ccccc2C(=O)OCc2ccc(CN)cc2)c(-c2cc(Cl)nc(N)n2)n1] | 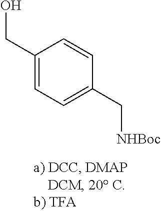 [SMILES: CC(C)(C)OC(=O)NCc1ccc(CO)cc1] | (CD3OD) δ 7.84 (dd, J = 1.3, 8.1 Hz, 1H), 7.48-7.42 (m, 1H), 7.32 (dd, J = 2.6, 7.4 Hz, 2H), 7.29 (s, 4H), 7.07 (s, 1H), 6.98 (s, 1H), 5.21 (s, 2H), 4.52 (s, 2H), 3.88 (s, 2H), 3.80 (s, 3H | 463.1 |
| 57 | 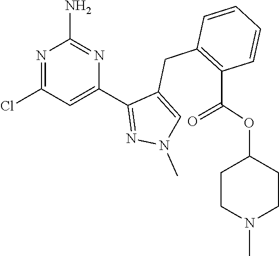 [SMILES: CN1CCC(OC(=O)c2ccccc2Cc2cn(C)nc2-c2cc(Cl)nc(N)n2)CC1] | 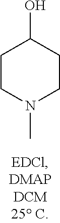 | (DMSO-d₆) δ 7.76 (d, J = 7.4 Hz, 1H), 7.51-7.45 (m, 1H), 7.38-7.30 (m, 2H), 7.11 (s, 1H), 7.00 (s, 3H), 4.90-4.78 (m, 1H), 4.52 (s, 2H), 3.79 (s, 3H), 2.44 (br s, 2H), 2.16 (br s, 2H), 2.12 (s, 3H), 1.81 (br dd, J = 3.2, 12.2 Hz, 2H), 1.63- 1.52 (m, 2H) | 441.1 |
| 62 | 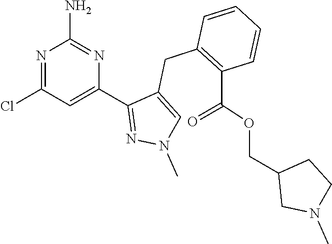 [SMILES: CN1CCC(COC(=O)c2ccccc2Cc2cn(C)nc2-c2cc(Cl)nc(N)n2)C1] | 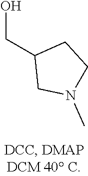 | (D₂O, HCl salt) δ 7.80-7.73 (m, 1H), 7.53-7.44 (m, 1H), 7.36-7.31 (m, 1H), 7.26 (d, J = 7.8 Hz, 1H), 7.12 (d, J = 8.9 Hz, 1H), 6.92 (d, J = 4.1 Hz, 1H), 4.27 (s, 2H), 4.18- 4.02 (m, 2H), 3.78 (s, 3H), 3.69-3.32 (m, 2H), 3.20-2.97 (m, 2H), 2.89-2.63 (m, 4H), 2.26-2.02 (m, 1H), 1.89-1.61 | 441.0 |
| | | | (m, 1H) | |

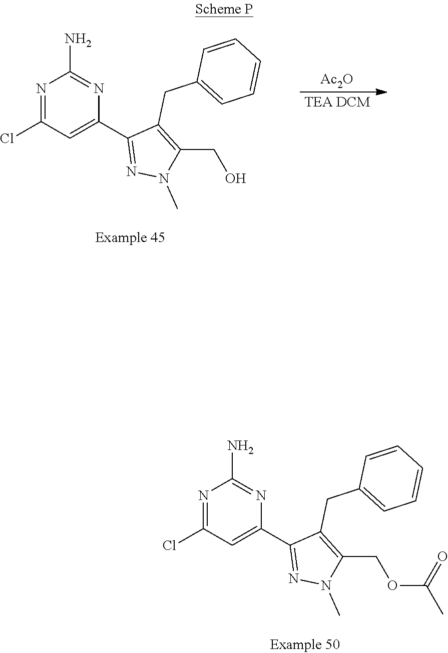 [SMILES: "Ac2O" ; Cn1nc(-c2cc(Cl)nc(N)n2)c(Cc2ccccc2)c1CO ; "TEA DCM" ; CC(=O)OCc1c(Cc2ccccc2)c(-c2cc(Cl)nc(N)n2)nn1C]

A mixture of [5-(2-amino-6-chloro-pyrimidin-4-yl)-4-benzyl-2-methyl-pyrazol-3-yl]methanol (80 mg, 0.24 mmol, 1 eq) in DCM (5 mL) was cooled to 0° C. Triethylamine (29 mg, 0.29 mmol, 1.2 eq) and Ac₂O (27 mg, 0.27 mmol, 1.1 eq) was added dropwise in that order. The mixture was allowed to warm to 15° C. and stir at that temperature for 3 h. The mixture was cooled to 0° C., and additional TEA (60 mg) and Ac₂O (55 mg) was added to the reaction. The mixture was allowed to warm to 15° C. and stir at that temperature for 12 h. The reaction mixture was concentrated under the reduced pressure. The residue was purified by neutral preparative-HPLC (column: Waters Xbridge BEH C18 100×25 mm, 5 m; mobile phase: [water (10 mM NH₄HCO₃)-ACN]; B %: 30%-60%, 8 min) which furnished [5-(2-amino-6-chloro-pyrimidin-4-yl)-4-benzyl-2-methyl-pyrazol-3-yl]methyl acetate Example 50.

Example 50: ¹H NMR: (CD3OD, 400 MHz) δ 7.21-7.12 (m, 4H), 7.11 (s, 1H), 7.10-7.06 (m, 1H), 5.12 (s, 2H), 4.42 (s, 2H), 3.96 (s, 3H), 1.92 (s, 3H); LCMS: (MH+) 372.1.

The following Examples in Table 7 were prepared in a similar fashion to that depicted in Scheme P using the appropriate conditions.

TABLE 7

| Ex. | Structure | Conditions | 1H NMR (400 MHz) | LCMS |
|---|---|---|---|---|
| 51 | 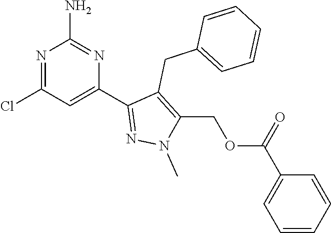 [SMILES: Cn1nc(-c2cc(Cl)nc(N)n2)c(Cc2ccccc2)c1COC(=O)c1ccccc1] | 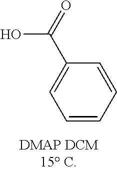 | (CD3OD δ 7.86 (d, J = 8.0 Hz, 2H), 7.64-7.56 (m, 1H), 7.48-7.40 (m, 2H), 7.20-7.08 (m, 5H), 7.06-6.98 (m, 1H), 5.39 (s, 2H), 4.49 (s, 2H), 4.05 (s, 3H) | 434.1 |

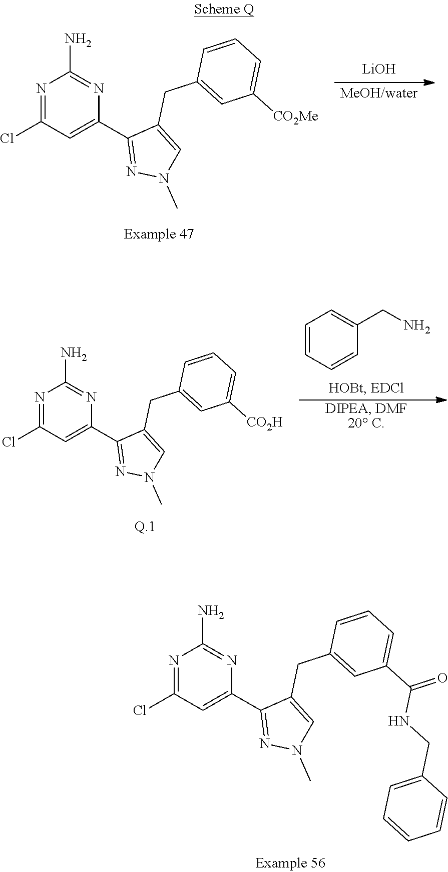

The intermediate acid Q.1 was prepared from Example 47 similar to that depicted in Scheme O for 0.1.

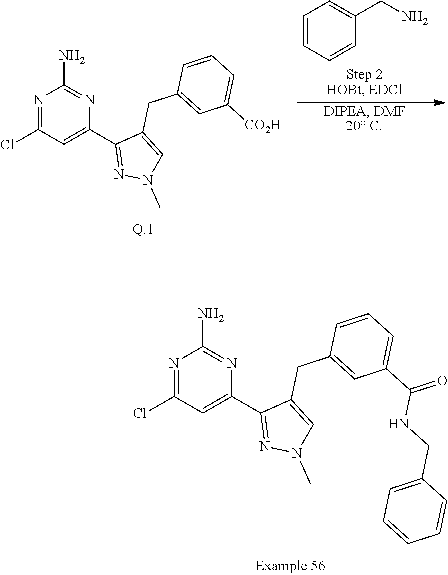

To a solution of 3-[[3-(2-amino-6-chloro-pyrimidin-4-yl)-1-methyl-pyrazol-4-yl] methyl]benzoic acid (100 mg, 0.291 mmol, 1 eq) in DMF (2 mL) was added HOBt (59 mg, 0.44 mmol, 1.5 eq), EDCI (84 mg, 0.44 mmol, 1.5 eq) and DIPEA (113 mg, 0.873 mmol, 3 eq). After stirring at 20° C. for 30 min, benzyl amine (47 mg, 0.44 mmol, 1.5 eq) was added. The reaction mixture was stirred at 20° C. for 12 hr under N₂. The reaction mixture was concentrated under reduced pressure. The residue was purified by preparative-HPLC (column: Waters Xbridge BEH C18 100×30 mm, 10 m; mobile phase: [water (10 mM NH₄HCO₃)-ACN]; B %: 35%-55%, 10 min, neutral condition) to afford 3-[[3-(2-amino-6-chloro-pyrimidin-4-yl)-1-methyl-pyrazol-4-yl] methyl]-N-methyl-benzamide Example 56.

Example 56: ¹H NMR: (400 MHz, DMSO-d₆) δ 8.95 (t, J=5.8 Hz, 1H), 7.80 (s, 1H), 7.67 (d, J=7.7 Hz, 1H), 7.52 (s, 1H), 7.42 (d, J=7.9 Hz, 1H), 7.36-7.26 (m, 5H), 7.18 (s, 1H), 7.11-7.04 (m, 2H), 6.96 (s, 1H), 4.44 (d, J=6.0 Hz, 2H), 4.31-4.26 (m, 2H), 3.83 (s, 3H); LCMS: (MH+) 433.2.

The examples in Table 8 were prepared in a similar fashion to that described in Scheme Q using the appropriate conditions for Step 2.

TABLE 8

| Ex. | Structure | Conditions | 1H NMR (400 MHz) | LCMS |
|---|---|---|---|---|
| 58 | 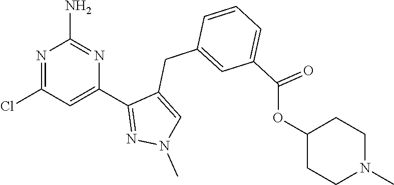 [SMILES: CN1CCC(OC(=O)c2cccc(Cc3cn(C)nc3-c3cc(Cl)nc(N)n3)c2)CC1] | 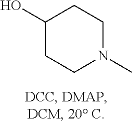 | (DMSO-d₆) δ 7.84 (s, 1H), 7.74 (br d, J = 7.7 Hz, 1H), 7.62-7.54 (m, 2H), 7.44-7.36 (m, 1H), 7.12-7.02 (m, 2H), 6.99 (s, 1H), 4.92-4.84 (m, 1H), 4.35 (s, 2H), 3.85 (s, 3H), 2.26-2.19 (m, 2H), 2.17 (s, 3H), 1.93- 1.81 (m, 3H), 1.74-1.62 (m, 3H) | 441.1 |
| 59 | 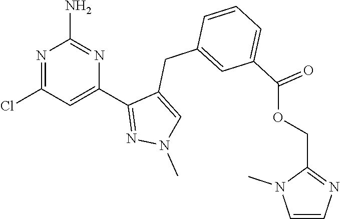 [SMILES: Cn1cc(Cc2cccc(C(=O)OCc3nccn3C)c2)c(-c2cc(Cl)nc(N)n2)n1] | 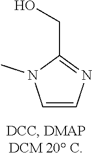 | (DMSO-d₆, formic acid salt) δ 8.14 (s, 0.5H), 7.82 (s, 1H), 7.74 (br d, J = 7.8 Hz, 1H), 7.59 (br d, J = 7.6 Hz, 1H), 7.56 (s, 1H), 7.40 (t, J = 7.6 Hz, 1H), 7.20 (s, 1H), 7.05 (br s, 2H), 6.98 (s, 1H), 6.87 (s, 1H), 5.33 (s, 2H), 4.35 (s, 2H), 3.84 (s, 3H), 3.69 (s, 3H) | 438.1 |
| 60 | 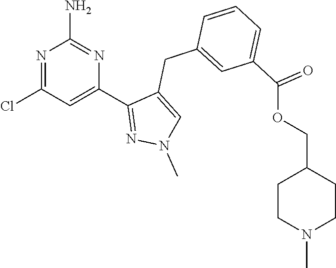 [SMILES: CN1CCC(COC(=O)c2cccc(Cc3cn(C)nc3-c3cc(Cl)nc(N)n3)c2)CC1] | 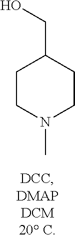 | (DMSO-d₆) δ 7.85 (s, 1H), 7.75 (d, J = 7.7 Hz, 1H), 7.62-7.56 (m, 2H), 7.45-7.38 (m, 1H), 7.07 (br s, 2H), 7.00 (s, 1H), 4.36 (s, 2H), 4.09 (d, J = 6.0 Hz, 2H), 3.85 (s, 3H), 2.77 (br d, J = 11.4 Hz, 2H), 2.15 (s, 3H), 1.85 (br t, J = 11.0 Hz, 2H), 1.69-1.59 (m, 3H), 1.27 (dq, J = 3.5, 12.4 Hz, 2H) | 455.1 |
| 61 | 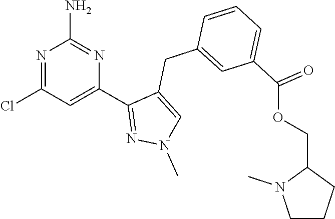 [SMILES: CN1CCCC1COC(=O)c1cccc(Cc2cn(C)nc2-c2cc(Cl)nc(N)n2)c1] | 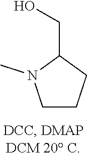 | (DMSO-d₆, formic acid salt) δ 8.18 (s, 1H), 7.81 (br s, 1H), 7.75 (br d, J = 7.7 Hz, 1H), 7.64- 7.58 (m, 2H), 7.45-7.38 (m, 1H), 7.08 (br s, 2H), 7.00 (s, 1H), 4.36 (s, 2H), 4.25-4.12 (m, 2H), 3.86 (s, 3H), 2.98-2.92 (m, 1H), 2.32 (s, 3H), 2.21 (br d, J = 8.9 Hz, 1H), 1.96-1.86 (m, 1H), 1.72-1.48 (m, 4H) | 441.1 |
| 63 | 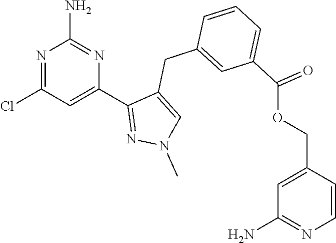 [SMILES: Cn1cc(Cc2cccc(C(=O)OCc3ccnc(N)c3)c2)c(-c2cc(Cl)nc(N)n2)n1] | 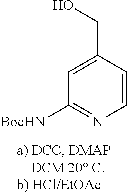 [SMILES: CC(C)(C)OC(=O)Nc1cc(CO)ccn1] | (DMSO-d₆) δ 7.91-7.86 (m, 2H), 7.82 (d, J = 7.8 Hz, 1H), 7.62 (br d, J = 7.8 Hz, 1H), 7.59 (s, 1H), 7.45-7.40 (m, 1H), 7.08 (br s, 2H), 6.99 (s, 1H), 6.47 (d, J = 5.3 Hz, 1H), 6.43-6.41 (m, 1H), 5.96 (s, 2H), 5.19 (s, 2H), 4.36 (s, 2H), 3.85 (s, 3H) | 450.1 |
| 64 | 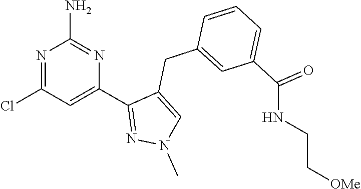 [SMILES: COCCNC(=O)c1cccc(Cc2cn(C)nc2-c2cc(Cl)nc(N)n2)c1] | 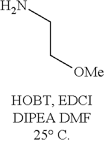 | (DMSO-d₆) δ 8.41 (br t, J = 5.3 Hz, 1H), 7.78 (s, 1H), 7.63 (d, J = 7.7 Hz, 1H), 7.53 (s, 1H), 7.42 (d, J = 7.9 Hz, 1H), 7.35-7.29 (m, 1H), 7.08 (br s, 2H), 6.99 (s, 1H), 4.29 (s, 2H), 3.85 (s, 3H), 3.46-3.38 (m, 4H), 3.25 (s, 3H) | 401.0 |

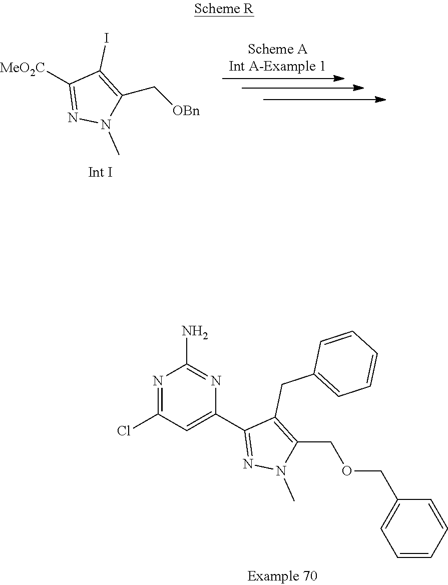 [SMILES: COC(=O)c1nn(C)c(COCc2ccccc2)c1I ; Cn1nc(-c2cc(Cl)nc(N)n2)c(Cc2ccccc2)c1COCc1ccccc1]

Intermediate I was converted into Example 70 using conditions similar to that depicted in Scheme A for Example 1 from Int A.

Example 70: ¹H NMR: (400 MHz, CD3OD) δ 7.36-7.24 (m, 5H), 7.19-7.13 (m, 2H), 7.12-7.05 (m, 4H), 4.52 (s, 2H), 4.43 (s, 2H), 4.32 (s, 2H), 3.91 (s, 3H); LCMS: (MH+) 420.2.

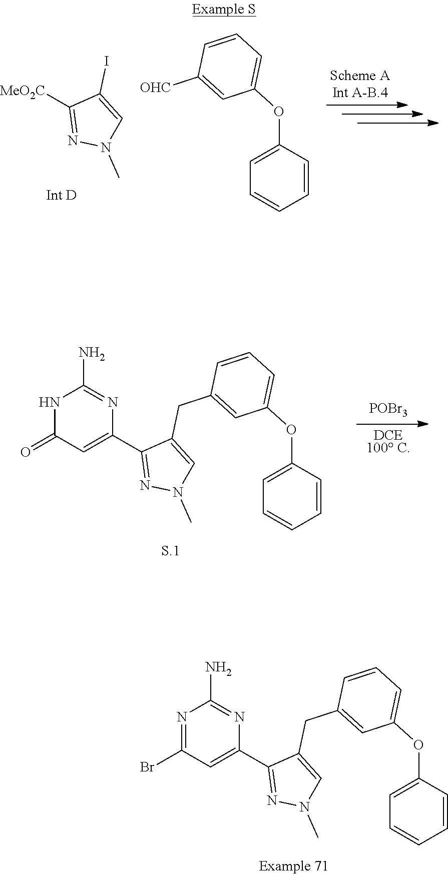 [SMILES: COC(=O)c1nn(C)cc1I ; O=Cc1cccc(Oc2ccccc2)c1 ; Cn1cc(Cc2cccc(Oc3ccccc3)c2)c(-c2cc(=O)[nH]c(N)n2)n1 ; "POBr3" ; "DCE100° C." ; Cn1cc(Cc2cccc(Oc3ccccc3)c2)c(-c2cc(Br)nc(N)n2)n1]

The intermediate S.1 was prepared from Int D and the aldehyde in a fashion similar to that depicted in Scheme B (Int A to B.4).

### Example 71

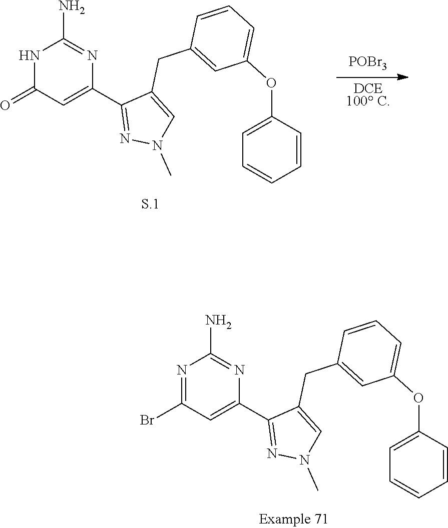 [SMILES: Cn1cc(Cc2cccc(Oc3ccccc3)c2)c(-c2cc(=O)[nH]c(N)n2)n1 ; "POBr3" ; "DCE100° C." ; Cn1cc(Cc2cccc(Oc3ccccc3)c2)c(-c2cc(Br)nc(N)n2)n1]

To a mixture of 2-amino-4-[1-methyl-4-[(3-phenoxyphenyl)methyl]pyrazol-3-yl]-1H-pyrimidin-6-one (100 mg, 0.268 mmol, 1 eq) in DCE (1 mL) was added POBr₃ (77 mg, 0.27 mmol, 1 eq). The mixture was stirred at 100° C. for 2 h under N₂. The reaction mixture was diluted with sat. aq. NaHCO₃ solution (30 mL). The solution was extracted with EtOAc (20 mL×3). The organic layer was washed with brine (40 mL), dried over Na₂SO₄, and filtered. The filtrate was concentrated under reduced pressure. The residue was purified by preparative-HPLC (column: Waters Xbridge BEH C18 100×30 mm, 10 m; mobile phase: [water (10 mM NH₄HCO₃)-ACN]; B %: 43%-63%, 10 min) which furnished 4-bromo-6-[1-methyl-4-[(3-phenoxyphenyl)methyl]pyrazol-3-yl]pyrimidin-2-amine Example 71.

Example 71: ¹H NMR: (400 MHz, CD3OD) δ 7.36 (s, 1H), 7.32-7.26 (m, 2H), 7.25-7.19 (m, 2H), 7.09-7.04 (m, 1H), 6.99 (d, J=7.5 Hz, 1H), 6.88 (d, J=7.8 Hz, 2H), 6.82 (s, 1H), 6.76 (dd, J=2.0, 8.1 Hz, 1H), 4.26 (s, 2H), 3.87 (s, 3H); LCMS: (MH+) 436.1.

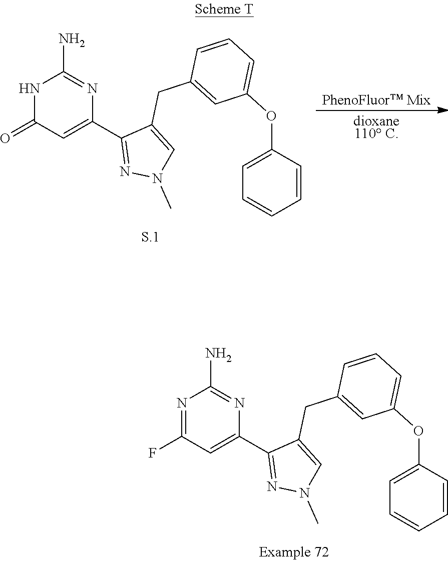

To a mixture of 2-amino-4-[1-methyl-4-[(3-phenoxyphenyl)methyl]pyrazol-3-yl]-1H-pyrimidin-6-one (100 mg, 0.268 mmol, 1 eq) in dioxane (1 mL) was added PhenoFluor™ Mix (550 mg). The mixture was stirred at 25° C. for 0.5 h and then at 110° C. for 36 h under N₂. Additional PhenoFluor™ Mix (300 mg) was added, and the mixture was stirred at 110° C. for another 12 h under N₂. The reaction mixture was diluted with water (100 mL). The mixture was extracted with EtOAc (50 mL×3). The organic layer was washed with brine (100 mL), dried over Na₂SO₄, and filtered. The filtrate was concentrated under reduced pressure. The residue was purified by gradient flash chromatography (SiO₂, petroleum ether/ethyl acetate=4/1 to 3/2). The residue was further purified by preparative-HPLC (column: Waters Xbridge BEH C18 100×25 mm, 5 m; mobile phase: [water (10 mM NH4HCO3)-ACN]; B %: 35%-70%, 8 min) which furnished fluoro-6-[1-methyl-4-[(3-phenoxyphenyl)methyl]pyrazol-3-yl]pyrimidin-2-amine (13.9 mg, 98.25% purity) Example 72.

Example 72: ¹H NMR: (400 MHz, CD3OD) δ 7.35 (s, 1H), 7.31-7.26 (m, 2H), 7.22 (t, J=7.9 Hz, 1H), 7.08-7.03 (m, 1H), 6.99 (d, J=7.6 Hz, 1H), 6.91-6.86 (m, 2H), 6.83 (t, J=1.7 Hz, 1H), 6.76 (dd, J=1.9, 8.1 Hz, 1H), 6.64 (s, 1H), 4.27 (s, 2H), 3.88 (s, 3H); LCMS: (MH+) 376.0

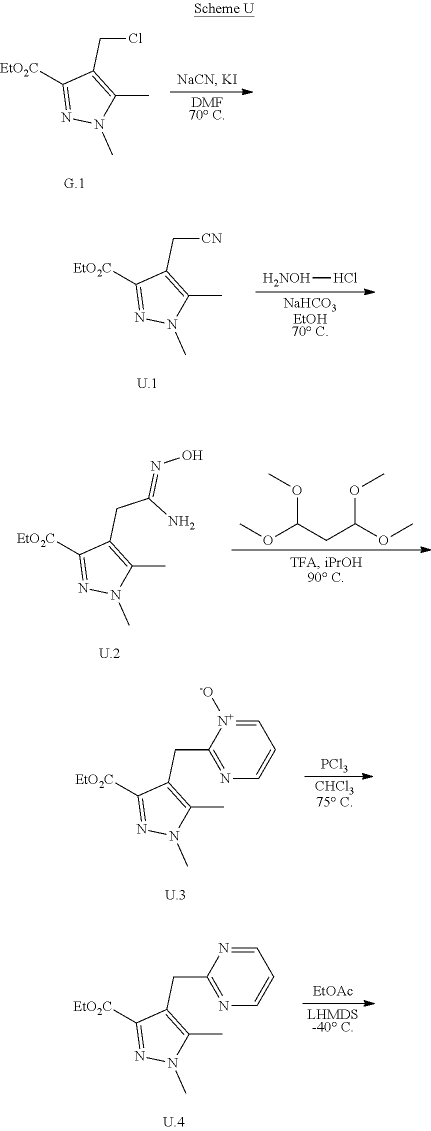

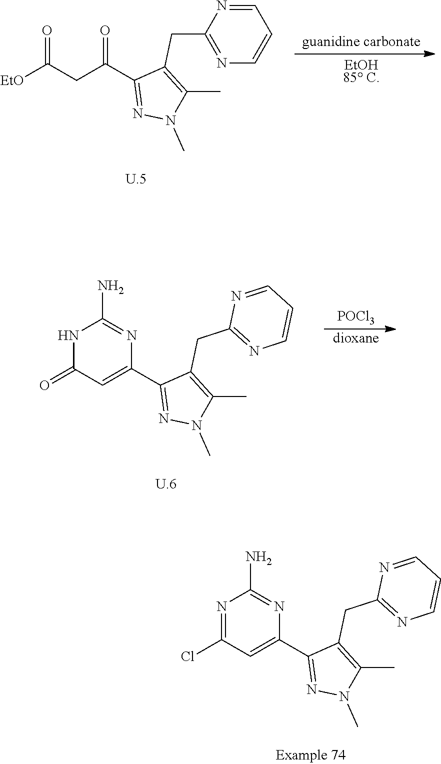

### Step 1

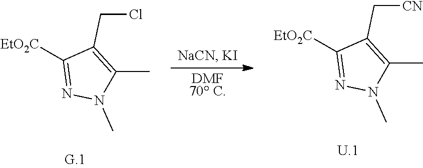

To a stirred mixture of ethyl 4-(chloromethyl)-1,5-dimethyl-pyrazole-3-carboxylate (6.35 g, 29.3 mmol, 1 eq) in DMF (60 mL) was cooled to 0° C. Sodium cyanide (1.72 g, 35.2 mmol, 1.2 eq) and KI (5.84 g, 35.2 mmol, 1.2 eq) was added to the reaction. The mixture was stirred at 70° C. for 12 h. The reaction mixture was diluted with water (150 mL). The mixture was adjusted to pH=11 by addition of aqueous NaOH (4M). The solution was extracted with DCM (60 mL×6). The organic layer was washed with brine (100 mL), dried over Na₂SO₄, and filtered. The filtrate was concentrated under reduced pressure. The residue was purified by column chromatography (SiO₂, petroleum ether/ethyl acetate=1/1). The residue was partitioned between water (200 mL) and ethyl acetate (70 mL). The mixture was extracted with ethyl acetate (70 mL×2). The combined organic layers were washed with brine (50 mL), dried over Na₂SO₄, and filtered. The filtrate was concentrated under reduced pressure to afford ethyl 4-(cyanomethyl)-1,5-dimethyl-pyrazole-3-carboxylate.

### Step 2

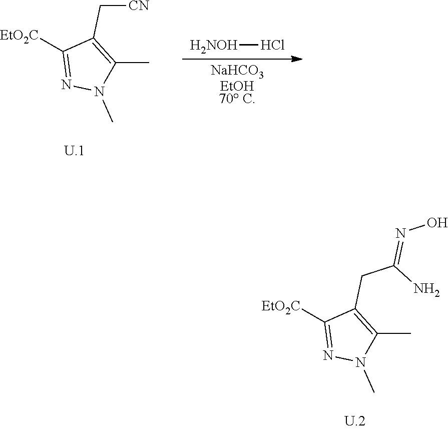

To a solution of ethyl 4-(cyanomethyl)-1,5-dimethyl-pyrazole-3-carboxylate (800 mg, 3.86 mmol, 1 eq) in EtOH (8 mL) was added NaHCO₃ (341 mg, 4.05 mmol, 1.05 eq) and NH₂OH·HCl (282 mg, 4.05 mmol, 1.05 eq) at 25° C. The mixture was stirred at 70° C. for 12 h. Additional NH₂OH·HCl (140 mg) and 1 NaHCO₃ (150 mg) were added to the mixture, and the reaction was stirred an additional 12 hr at 70° C. The reaction mixture was concentrated under the reduced pressure. The reaction mixture was filtered, and the residue was washed with ethyl acetate (10 mL×5) and EtOH (10 mL×5). Then the filtrate was concentrated under reduced pressure which furnished ethyl 4-[(2Z)-2-amino-2-hydroxyimino-ethyl]-1,5-dimethyl-pyrazole-3-carboxylate.

### Step 3

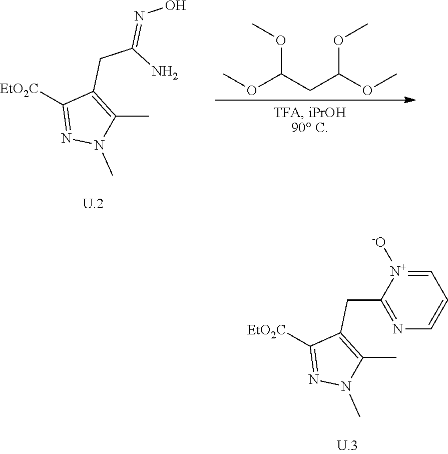

To a stirred mixture of ethyl 4-[(2Z)-2-amino-2-hydroxyimino-ethyl]-1,5-dimethyl-pyrazole-3-carboxylate (900 mg, 3.75 mmol, 1 eq), 1,1,3,3-tetramethoxypropane (1.23 g, 7.49 mmol, 1.24 mL, 2 eq) and TFA (513 mg, 4.50 mmol, 0.333 mL, 1.2 eq) in 2-propanol (18 mL) was heated at 90° C. for 12 h. The reaction mixture was adjusted to pH=7 by addition of sat. aqueous NaHCO₃. The solution was concentrated under reduced pressure to furnish around 10 ml total volume. The solution was purified by preparative-HPLC (column: Welch Xtimate C18 250×50 mm, 10 m; mobile phase: [water (0.04% NH₃H₂O+10 mM NH₄HCO₃)-ACN]; B %: 5%-30%, 10 min) which furnished ethyl 1,5-dimethyl-4-[(1-oxi)dopyrimidin-1-ium-2-yl)methyl]pyrazole-3-carboxylate.

### Step 4

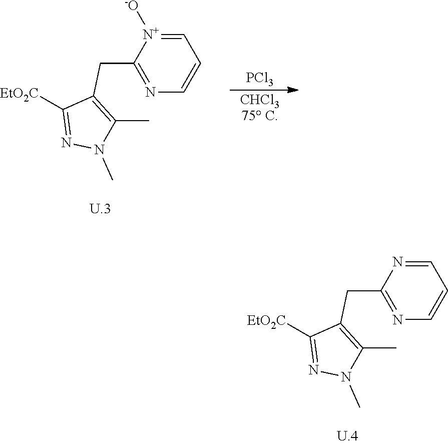

PCl₃ (418 mg, 3.04 mmol, 2.1 eq) was added into a solution of ethyl 1,5-dimethyl-4-[(1-oxidopyrimidin-1-ium-2-yl)methyl]pyrazole-3-carboxylate (400 mg, 1.45 mmol, 1 eq) in chloroform (8 mL) at 25° C. The mixture was stirred at 75° C. for 35 min. The reaction mixture was diluted with sat. aqueous NaHCO₃ (100 mL). The solution was extracted with ethyl acetate (40 mL×6). The combined organic layers were washed with brine (50 mL), dried over Na₂SO₄, and filtered. The filtrate was concentrated under the reduced pressure. The residue was purified by column chromatography (SiO₂, petroleum ether/ethyl acetate/MeOH=2/1/0 to 0/1/0 to 0/9/1 gradient) to afford ethyl 1,5-dimethyl-4-(pyrimidin-2-ylmethyl)pyrazole-3-carboxylate.

### Example 74

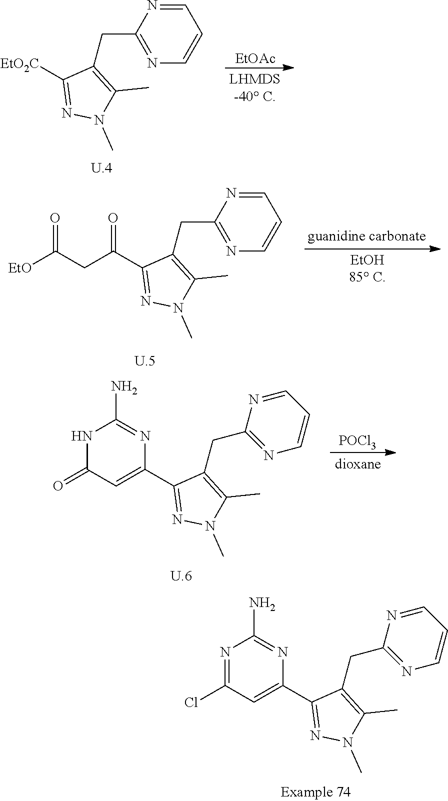

Example 74 was prepared from U.4 using conditions similar to that depicted in Scheme C for Example 3 from C.3.

Example 74: ¹H NMR: (DMSO-d6, 400 MHz) δ 8.64 (d, J=4.9 Hz, 2H), 7.26 (t, J=4.8 Hz, 1H), 6.99 (s, 1H), 6.80 (br s, 2H), 4.63 (s, 2H), 3.79 (s, 3H), 2.09 (s, 3H); LCMS: (MH+) 316.1.

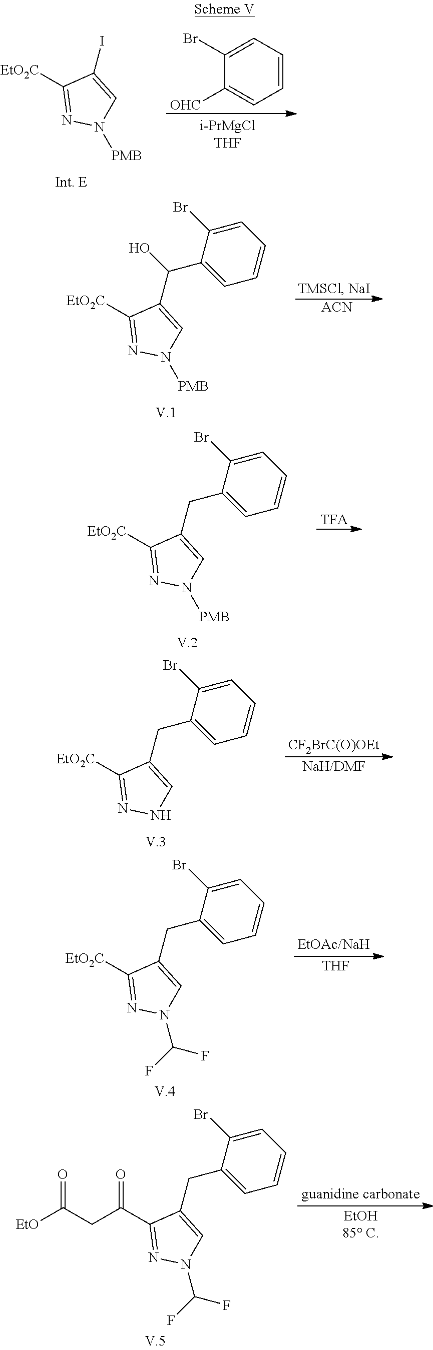

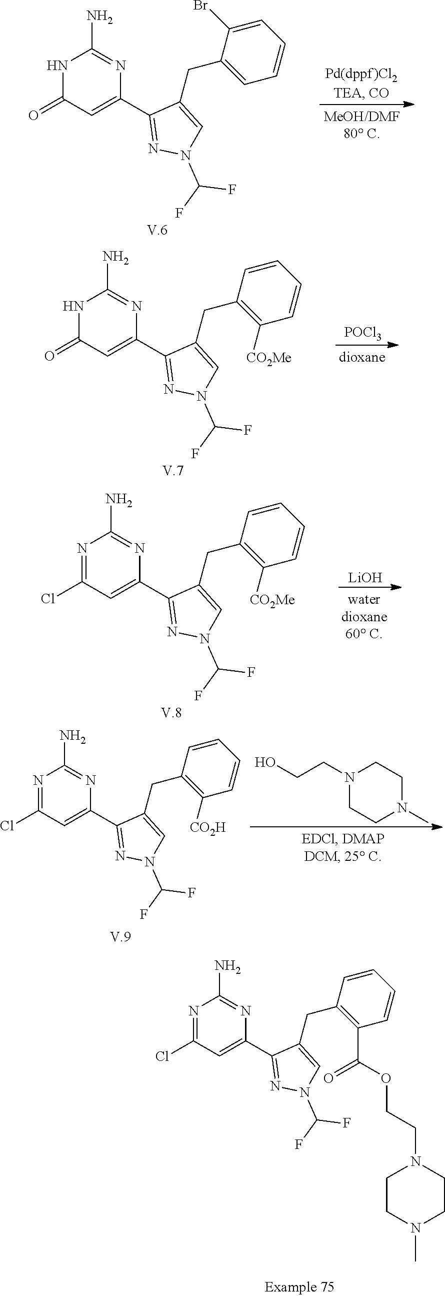

Intermediate V.6 was prepared from Int. E using conditions similar to those outlined in Scheme E for E.6.

### Step 7

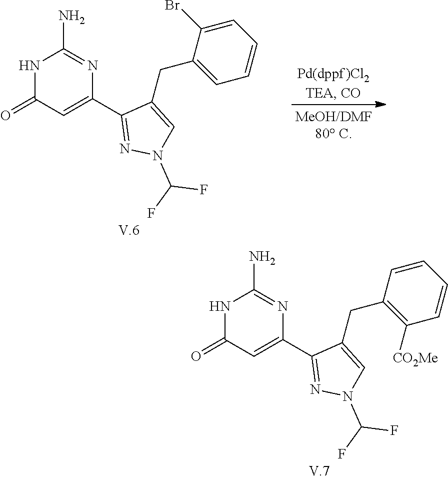 [SMILES: Nc1nc(-c2nn(C(F)F)cc2Cc2ccccc2Br)cc(=O)[nH]1 ; "Pd(dppf)Cl2TEA, CO" ; COC(=O)c1ccccc1Cc1cn(C(F)F)nc1-c1cc(=O)[nH]c(N)n1]

To a solution of 2-amino-4-[4-[(2-bromophenyl)methyl]-1-(difluoromethyl)pyrazol-3-yl]-1H-pyrimidin-6-one (190 mg, 0.480 mmol, 1 eq) in MeOH (9 mL) and DMF (6 mL) was added Pd(dppf)Cl₂ (70 mg, 0.096 mmol, 0.2 eq) and TEA (194 mg, 1.92 mmol, 0.267 mL, 4 eq) under N₂. The suspension was degassed and purged with CO (3 cycles). The mixture was stirred under CO (50 psi) at 80° C. for 48 h. The reaction mixture was filtered, and the filtrate was concentrated. The residue was dissolved in ethyl acetate (20 mL). The organic phase was washed with brine (80 mL), dried over anhydrous Na₂SO₄, and filtered. The filtrate was concentrated which furnished methyl 2-[[3-(2-amino-6-oxo-1H-pyrimidin-4-yl)-1-(difluoro methyl)pyrazol-4-yl]methyl]benzoate.

### Step 8

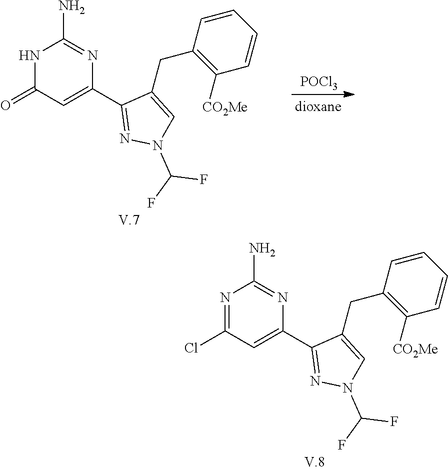

To a solution of methyl 2-[[3-(2-amino-6-oxo-1H-pyrimidin-4-yl)-1-(difluoromethyl) pyrazol-4-yl]methyl]benzoate (300 mg, 0.799 mmol, 1 eq) in dioxane (3 mL) was added POCl₃ (1.84 g, 12.0 mmol, 1.11 mL, 15 eq). The mixture was stirred at 75° C. for 12 h under N₂. The reaction mixture was poured into a saturated, aqueous sodium bicarbonate solution (150 mL). The mixture was extracted with ethyl acetate (50 mL×3). The organic phase was washed with brine (80 mL), dried over anhydrous Na₂SO₄, and filtered. The filtrate was concentrated. The crude product was purified by flash chromatography (ISCO®; 4 g SepaFlash@ Silica Flash Column, gradient of 0-15% ethyl acetate/petroleum @ 75 mL/min) to give methyl 2-[[3-(2-amino-6-chloro-pyrimidin-4-yl)-1-(difluoromethyl)pyrazol-4-yl]methyl]benzoate.

### Step 9

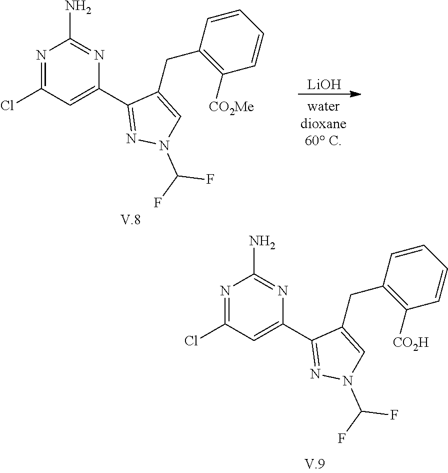 [SMILES: COC(=O)c1ccccc1Cc1cn(C(F)F)nc1-c1cc(Cl)nc(N)n1 ; "LiOH" ; Nc1nc(Cl)cc(-c2nn(C(F)F)cc2Cc2ccccc2C(=O)O)n1]

To a solution of methyl 2-[[3-(2-amino-6-chloro-pyrimidin-4-yl)-1-(difluoro methyl)pyrazol-4-yl]methyl]benzoate (50 mg, 0.123 mmol, 1 eq) in dioxane (1.5 mL) and H₂O (0.3 mL) was added LiOH·H₂O (80 mg, 1.9 mmol, 15 eq). The mixture was stirred at 60° C. for 12 h under N₂. The reaction mixture was poured into H₂O (20 mL). The pH of the mixture was adjusted to 3 by addition of aqueous 1N HCl. The mixture was filtered, and the filter cake was collected and dried which furnished 2-[[3-(2-amino-6-chloro-pyrimidin-4-yl)-1-(difluoromethyl) pyrazol-4-yl]methyl]benzoic acid (50 mg).

### Step 10

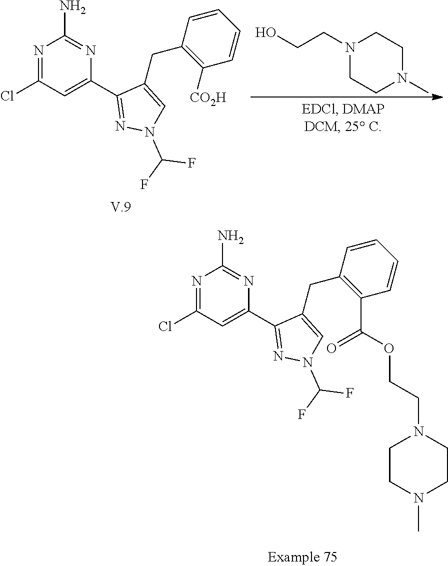

To a solution of 2-[[3-(2-amino-6-chloro-pyrimidin-4-yl)-1-(difluoromethyl)pyrazol-4-yl]methyl]benzoic acid (50 mg, 0.13 mmol, 1 eq) in DCM (2 mL) was added EDCI (30 mg, 0.16 mmol, 1.2 eq) and DMAP (19 mg, 0.16 mmol, 1.2 eq) at 0° C. After stirring the mixture for 5 minutes, 2-(4-methylpiperazin-1-yl)ethanol (38 mg, 0.26 mmol, 2 eq) was added at 0° C. The mixture was stirred at 25° C. for 12 h under N₂. The reaction mixture was poured into H₂O (100 mL). The mixture was extracted with ethyl acetate (30 mL×3). The organic phase was washed with brine (30 mL), dried over anhydrous Na₂SO₄, and filtered. The filtrate was concentrated. The residue was purified by preparative-HPLC (column: Waters Xbridge BEH C18 100×25 mm, 5 m; mobile phase: [water (10 mM NH₄HCO₃)-ACN]; B %:30%-60%, 8 min) to give 2-(4-methylpiperazin-1-yl)ethyl 2-[[3-(2-amino-6-chloro-pyrimidin-4-yl)-1-(difluoromethyl)pyrazol-4-yl]methyl]benzoate Example 75.

Example 75: ¹H NMR: (400 MHz, CD3OD) δ 7.84 (d, J=7.8 Hz, 1H), 7.63-7.42 (m, 3H), 7.34-7.31 (m, 1H), 7.31-7.28 (m, 1H), 7.22 (s, 1H), 4.64 (s, 2H), 4.36 (t, J=5.7 Hz, 2H), 2.73-2.25 (m, 10H), 2.21 (s, 3H); LCMS: (MH+) 506.2.

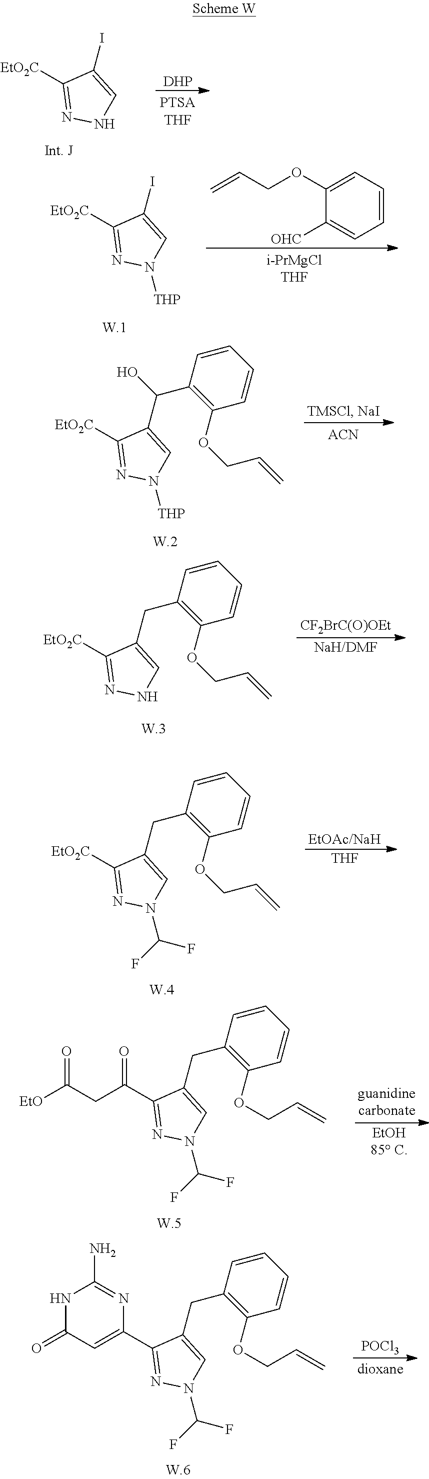

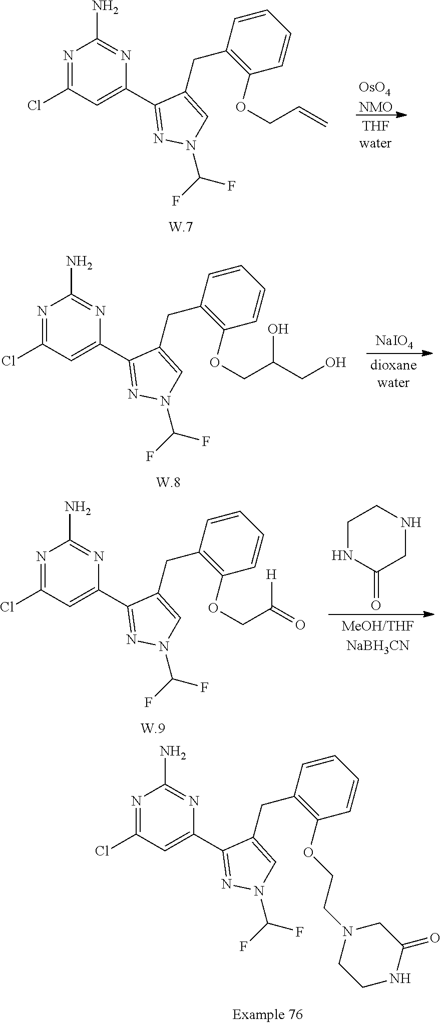

### Step 1

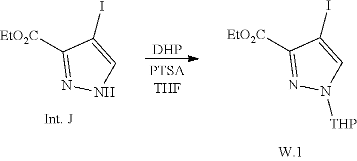

A solution of ethyl 4-iodo-1H-pyrazole-3-carboxylate (20 g, 75 mmol, 1 eq) in THF (75 mL) was added DHP (19.0 g, 226 mmol, 20.6 mL, 3 eq) and PTSA (1.29 g, 7.52 mmol, 0.1 eq). The reaction mixture was stirred at 80° C. for 8 hr under N₂. The reaction mixture was concentrated under reduced pressure. Then mixture was diluted with H₂O (300 mL) and extracted with EtOAc (100 mL×3). The combined organic layers were washed with brine (100 mL), dried over Na₂SO₄, and filtered. The filtrate was concentrated under reduced pressure. The residue was purified by flash silica gel chromatography (ISCO®; 20 g SepaFlash@ Silica Flash Column, gradient eluent of 0 to 20% EtOAc/petroleum ether @100 mL/min) which furnished 4-iodo-1-tetrahydropyran-2-yl-pyrazole-3-carboxylate.

The intermediate W.1 was converted into W.7 using conditions similar those outlined in for Example 18 in Scheme E.

### Step 8

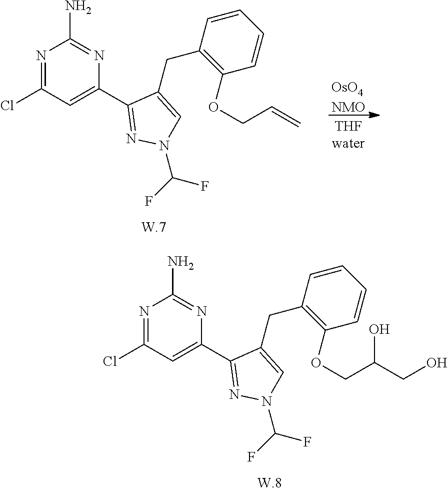

To a solution of 4-[4-[(2-allyloxyphenyl)methyl]-1-(difluoromethyl)pyrazol-3-yl]-6-chloro-pyrimidin-2-amine (100 mg, 0.255 mmol, 1 eq) in THF (1 mL) and H₂O (1 mL) was added NMO (84 mg, 0.71 mmol, 0.075 mL, 2.8 eq) and OSO₄ (13 mg, 0.051 mmol, 0.2 eq) at 0° C. The reaction mixture was stirred at 25° C. for 2 hr under N₂. The reaction mixture was quenched with saturated aqueous Na₂SO₃ (40 mL). The mixture was extracted with EtOAc (20 mL×3). The organic layer was washed with brine (20 mL), dried over Na₂SO₄, and filtered. The filtrate was concentrated under reduced pressure. The residue was purified by flash chromatography (ISCO®; 4 g SepaFlash@ Silica Flash Column, gradient of 0 to 60% EtOAc/petroleum ether @ 50 mL/min) which furnished 3-[2-[[3-(2-amino-6-chloro-pyrimidin-4-yl)-1-(difluoromethyl)pyrazol-4-yl]methyl]phenoxy]propane-1,2-diol.

### Step 9

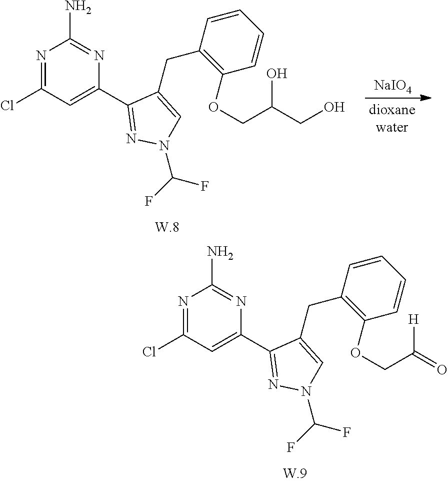 [SMILES: Nc1nc(Cl)cc(-c2nn(C(F)F)cc2Cc2ccccc2OCC(O)CO)n1 ; "NaIO4" ; Nc1nc(Cl)cc(-c2nn(C(F)F)cc2Cc2ccccc2OCC=O)n1]

A mixture of 3-[2-[[3-(2-amino-6-chloro-pyrimidin-4-yl)-1-(difluoromethyl) pyrazol-4-yl]methyl]phenoxy]propane-1,2-diol (70 mg, 0.16 mmol, 1 eq) in dioxane (1 mL) and H₂O (0.3 mL) was added NaIO₄ (88 mg, 0.41 mmol, 2.5 eq) at 0° C. The reaction mixture was stirred at 25° C. for 2 hr under N₂. The reaction mixture was quenched with saturated aqueous Na₂SO₃ (15 mL). The mixture was extracted with EtOAc (8 mL×3). The combined organic layer was washed with brine (10 mL), dried over Na₂SO₄, and filtered. The filtrate was concentrated under reduced pressure which furnished 2-[2-[[3-(2-amino-6-chloro-pyrimidin-4-yl)-1-(difluoromethyl)pyrazol-4-yl]methyl]phenoxy]acetaldehyde.

### Step 10

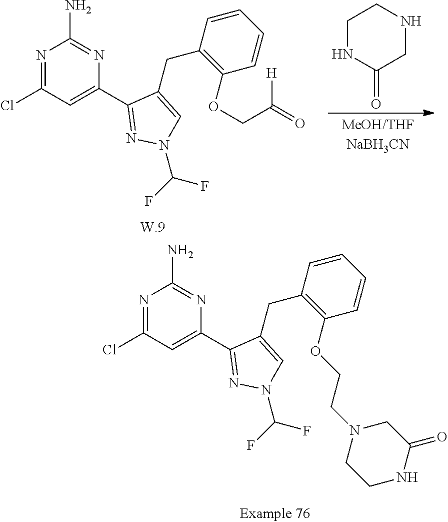 [SMILES: Nc1nc(Cl)cc(-c2nn(C(F)F)cc2Cc2ccccc2OCC=O)n1 ; O=C1CNCCN1 ; Nc1nc(Cl)cc(-c2nn(C(F)F)cc2Cc2ccccc2OCCN2CCNC(=O)C2)n1]

To a mixture of 2-[2-[[3-(2-amino-6-chloro-pyrimidin-4-yl)-1-(difluoromethyl) pyrazol-4-yl]methyl]phenoxy]acetaldehyde (70 mg, 0.18 mmol, 1 eq) and piperazin-2-one (71 mg, 0.71 mmol, 4 eq) in MeOH (2 mL) and THF (1 mL) was added AcOH (11 mg, 0.18 mmol, 1 eq). After stirring at 25° C. for 2 h, NaBH₃CN (45 mg, 0.71 mmol, 4 eq) was added to the mixture, and the resulting mixture was stirred at 25° C. for 2 h under N₂. The reaction mixture was quenched with saturated aqueous NaHCO₃ (20 mL). The mixture was extracted with EtOAc (20 mL×3). The organic layer was washed with brine (30 mL), dried over Na₂SO₄, and filtered. The filtrate was concentrated under reduced pressure. The residue was purified by preparative-HPLC (column: Welch Xtimate C18 150×30 mm, 5 m; mobile phase: [water (10 mM NH₄HCO₃)-ACN]; B %: 30%-60%, 3 min; neutral condition) which furnished 4-[2-[2-[[3-(2-amino-6-chloro-pyrimidin-4-yl)-1-(difluoromethyl)pyrazol-4-yl]methyl]phenoxy]ethyl]piperazin-2-one Example 76.

Example 76: ¹H NMR: (400 MHz, DMSO-d₆) δ 7.92-7.60 (m, 3H), 7.28 (s, 2H), 7.24-7.15 (m, 2H), 7.02 (s, 1H), 6.98 (d, J=8.3 Hz, 1H), 6.85 (t, J=7.4 Hz, 1H), 4.23 (s, 2H), 4.07 (br t, J=5.4 Hz, 2H), 3.06 (br s, 2H), 3.01 (s, 2H), 2.71-2.67 (m, 2H), 2.60-2.56 (m, 2H); LCMS: (MH+) 478.0.

The following examples in Table 9 were prepared in a similar to that depicted in Scheme W using the appropriate reagents for Step 10.

TABLE 9

| Ex. | Structure | Conditions | 1H NMR (400 MHz) | LCMS |
|---|---|---|---|---|
| 77 | 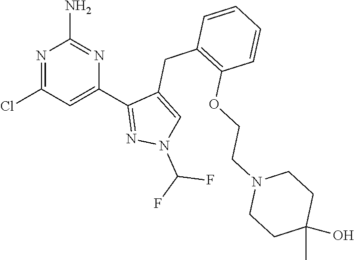 [SMILES: CC1(O)CCN(CCOc2ccccc2Cc2cn(C(F)F)nc2-c2cc(Cl)nc(N)n2)CC1] | 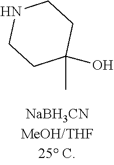 | (DMSO-d₆) δ 7.93-7.61 (m, 2H), 7.28 (s, 2H), 7.24 (dd, J = 1.4, 7.4 Hz, 1H), 7.20-7.14 (m, 1H), 7.02 (s, 1H), 6.96 (d, J = 8.0 Hz, 1H), 6.84 (t, J = 7.2 Hz, 1H), 4.22 (s, 2H), 4.06 (s, 1H), 4.01 (t, J = 5.8 Hz, 2H), 2.60-2.55 (m, 2H), 2.39 (br d, J = 4.8 Hz, 4H), 1.39 (br t, J = 5.4 Hz, 4H), 1.05 (s, 3H) | 493.1 |
| 80 | 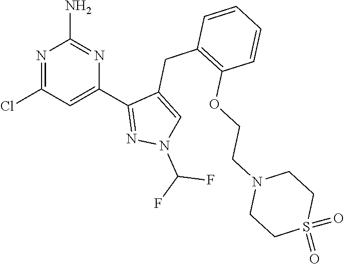 [SMILES: Nc1nc(Cl)cc(-c2nn(C(F)F)cc2Cc2ccccc2OCCN2CCS(=O)(=O)CC2)n1] | 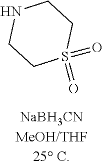 | (DMSO-d₆) δ 7.94-7.61 (m, 2H), 7.30-7.16 (m, 4H), 7.03 (s, 1H), 6.98 (d, J = 8.3 Hz, 1H), 6.86 (t, J = 7.4 Hz, 1H), 4.22 (s, 2H), 4.05 (t, J = 5.3 Hz, 2H), 2.97 (br d, J = 7.0 Hz, 8H), 2.84 (t, J = 5.3 Hz, 2H) | 513.1 |
| 86 | 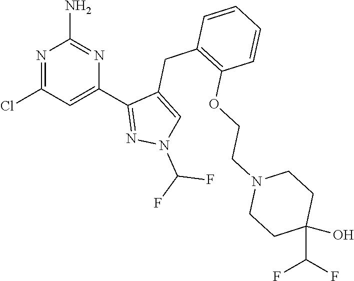 [SMILES: Nc1nc(Cl)cc(-c2nn(C(F)F)cc2Cc2ccccc2OCCN2CCC(O)(C(F)F)CC2)n1] | 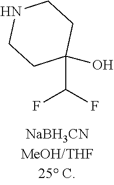 | (DMSO-d₆) δ 7.93-7.61 (m, 2H), 7.31-7.25 (m, 2H), 7.25-7.14 (m, 2H), 7.02 (s, 1H), 6.97 (d, J = 8.0 Hz, 1H), 6.84 (t, J = 7.3 Hz, 1H), 5.78-5.47 (m, 1H), 4.97 (s, 1H), 4.22 (s, 2H), 4.03 (t, J = 5.8 Hz, 2H), 2.67 (br d, J = 1.9 Hz, 2H), 2.61 (t, J = 5.8 Hz, 2H), 2.28 (dt, J = 2.4, 11.7 Hz, 2H), 1.51 (dt, J = 4.2, 12.7 Hz, 2H), 1.44-1.37 (m, 2H) | 529.1 |
| 91 | 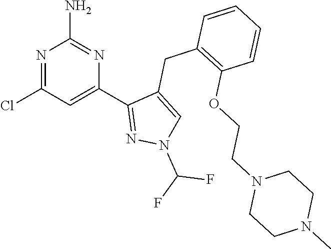 [SMILES: CN1CCN(CCOc2ccccc2Cc2cn(C(F)F)nc2-c2cc(Cl)nc(N)n2)CC1] | 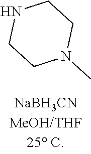 | (CD3OD, HCl salt) δ 7.68-7.35 (m, 2H), 7.33- 7.27 (m, 1H), 7.24 (s, 1H), 7.21-7.15 (m, 1H), 7.09 (d, J = 8.3 Hz, 1H), 7.03-6.96 (m, 1H), 4.49- 4.43 (m, 2H), 4.32 (s, 2H), 3.71-3.50 (m, 10H), 2.97 (s, 3H) | 478.2 |
| 122 | 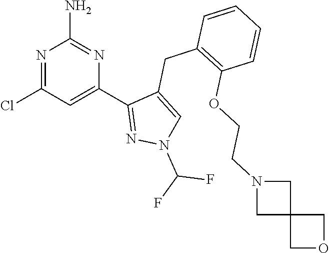 [SMILES: Nc1nc(Cl)cc(-c2nn(C(F)F)cc2Cc2ccccc2OCCN2CC3(COC3)C2)n1] | 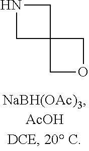 | (, DMSO-d₆) δ 7.95-7.63 (m, 2H), 7.34-7.29 (m, 2H), 7.23-7.14 (m, 2H), 7.04 (s, 1H), 6.91 (d, J = 8.1 Hz, 1H), 6.85 (t, J = 7.4 Hz, 1H), 4.50-4.45 (m, 4H), 4.22 (s, 2H), 3.87 (t, J = 5.0 Hz, 2H), 3.22 (s, 4H), 2.59 (br t, J = 4.8 Hz, 2H) | 477.1 |
| 124 | 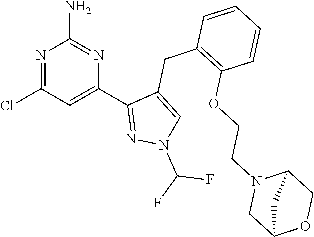 [SMILES: Nc1nc(Cl)cc(-c2nn(C(F)F)cc2Cc2ccccc2OCCN2C[C@H]3C[C@@H]2CO3)n1] | 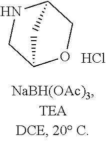 | (CDCl3) δ 7.55 (s, 1H), 7.31-7.27 (m, 1H), 7.23- 6.84 (m, 5H), 5.44-5.33 (m, 2H), 4.38-4.28 (m, 2H), 4.24-4.16 (m, 1H), 4.14-3.99 (m, 3H), 3.58 (dd, J = 1.4, 7.9 Hz, 1H), 3.55-3.51 (m, 1H), 3.07- 2.90 (m, 3H), 2.64 (d, J = 10.3 Hz, 1H), 1.83 (br d, J = 9.0 Hz, 1H), 1.70 (br d, J = 9.9 Hz, 1H) | 477.1 |
| 131 | 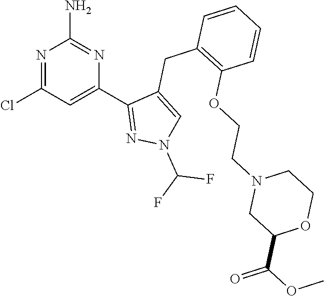 [SMILES: COC(=O)[C@H]1CN(CCOc2ccccc2Cc2cn(C(F)F)nc2-c2cc(Cl)nc(N)n2)CCO1] | 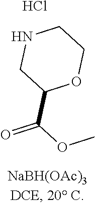 | (CDCl3) δ 7.52-7.48 (m, 1H), 7.32-7.28 (m, 1H), 7.22 (dt, J = 1.5, 7.8 Hz, 1H), 7.17-7.15 (m, 0.5H), 7.13 (dd, J = 1.1, 7.3 Hz, 1H), 7.01 (s, 0.3H), 6.94- 6.87 (m, 2H), 5.27 (br s, 2H), 4.25 (s, 2H), 4.20 (dd, J = 2.9, 9.1 Hz, 1H), 4.13 (t, J = 5.5 Hz, 2H), 3.99-3.92 (m, 1H), 3.74 (s, 3H), 3.69-3.61 (m, 1H), 3.10-3.03 (m, 1H), 2.78 (t, J = 5.4 Hz, 2H), 2.66 (br d, J = 11.4 Hz, 1H), 2.48-2.34 (m, 2H) | 523.2 |
| 132 | 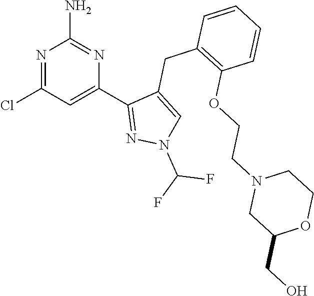 [SMILES: Nc1nc(Cl)cc(-c2nn(C(F)F)cc2Cc2ccccc2OCCN2CCO[C@@H](CO)C2)n1] | 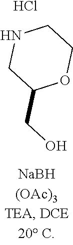 | (CDCl3) δ 7.49 (s, 1H), 7.32-7.28 (m, 1H), 7.25- 7.19 (m, 1H), 7.17-7.16 (m, 0.5H), 7.14 (dd, J = 1.3, 7.4 Hz, 1H), 7.01 (s, 0.2H), 6.94-6.87 (m, 2H), 5.38-5.23 (m, 2H), 4.30-4.19 (m, 2H), 4.13 (t, J = 5.4 Hz, 2H), 3.89- 3.82 (m, 1H), 3.69-3.51 (m, 4H), 2.82-2.68 (m, 4H), 2.26 (dt, J = 3.3, 11.3 Hz, 1H), 2.18-2.10 (m, 1H), 2.03 (br s, 1H) | 495.2 |
| 133 | 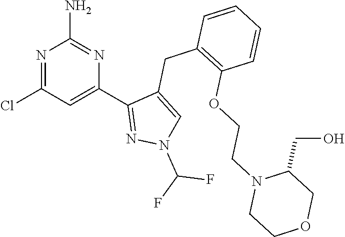 [SMILES: Nc1nc(Cl)cc(-c2nn(C(F)F)cc2Cc2ccccc2OCCN2CCOC[C@H]2CO)n1] | 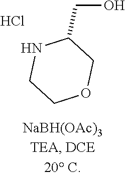 | (CDCl3) δ 7.43 (s, 1H), 7.32-7.27 (m, 1H), 7.25- 7.19 (m, 1H), 7.16 (s, 0.5H), 7.11-7.06 (m, 1H), 7.01 (s, 0.3H), 6.95-6.88 (m, 2H), 5.53-5.37 (m, 2H), 4.35-4.20 (m, 2H), 4.19-4.11 (m, 1H), 4.10- 4.02 (m, 1H), 3.85 (dd, J = 4.1, 11.6 Hz, 1H), 3.80-3.70 (m, 2H), 3.62- 3.46 (m, 2H), 3.45-3.36 (m, 1H), 3.29-3.20 (m, 1H), 2.90 (td, J = 2.7, 11.8 Hz, 1H), 2.79-2.66 | 495.2 |
| | | | (m, 2H), 2.59-2.48 (m, | |
| | | | 2H) | |
| 135 | 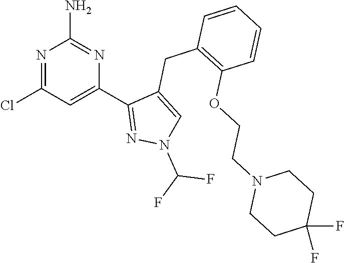 [SMILES: Nc1nc(Cl)cc(-c2nn(C(F)F)cc2Cc2ccccc2OCCN2CCC(F)(F)CC2)n1] | 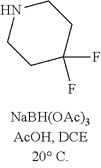 | (CDCl3) δ 7.42 (s, 1H), 7.23 (s, 0.2H), 7.22 (s, 0.7H), 7.15 (dt, J = 1.6, 7.8 Hz, 1H), 7.10-7.05 (m, 1.39H), 6.92 (s, 0.24H), 6.86-6.79 (m, 2H), 5.15 (br s, 2H), 4.17 (s, 2H), 4.02 (t, J = 5.5 Hz, 2H), 2.73 (t, J = 5.5 Hz, 2H), 2.54 (br t, J = 5.6 Hz, 4H), 1.93-1.78 (m, 4H) | 499.2 |
| 136 | 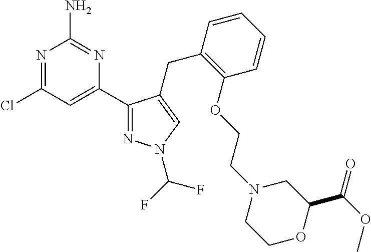 [SMILES: COC(=O)[C@@H]1CN(CCOc2ccccc2Cc2cn(C(F)F)nc2-c2cc(Cl)nc(N)n2)CCO1] | 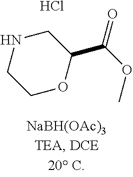 | (CDCl3) δ 7.50 (s, 1H), 7.31 (s, 0.2H), 7.30 (s, 1H), 7.25-7.19 (m, 1H), 7.16 (s, 0.4H), 7.13 (dd, J = 1.4, 7.3 Hz, 1H), 7.01 (s, 0.2H), 6.93-6.87 (m, 2H), 5.26 (br s, 2H), 4.26 (s, 2H), 4.20 (dd, J = 2.9, 9.1 Hz, 1H), 4.13 (t, J = 5.4 Hz, 2H), 3.96 (td, J = 3.3, 11.4 Hz, 1H), 3.74 (s, 3H), 3.69-3.61 (m, 1H), 3.06 (br d, J = 11.4 Hz, 1H), 2.78 (t, J = 5.5 Hz, 2H), 2.66 (br d, J = 11.4 Hz, 1H), 2.49-2.33 | 523.3 |
| | | | (m, 2H) | |
| 137 | 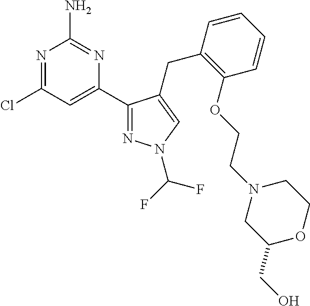 [SMILES: Nc1nc(Cl)cc(-c2nn(C(F)F)cc2Cc2ccccc2OCCN2CCO[C@H](CO)C2)n1] | 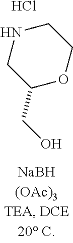 | (CDCl3) δ 7.49 (s, 1H), 7.31 (s, 0.2H), 7.30 (s, 1H), 7.22 (dt, J = 1.6, 7.8 Hz, 1H), 7.16 (s, 0.4H), 7.14 (dd, J = 1.4, 7.3 Hz, 1H), 7.01 (s, 0.2H), 6.93-6.87 (m, 2H), 5.28 (br s, 2H), 4.25 (d, J = 3.3 Hz, 2H), 4.13 (t, J = 5.4 Hz, 2H), 3.89- 3.81 (m, 1H), 3.70-3.49 (m, 4H), 2.84-2.67 (m, 4H), 2.27 (dt, J = 3.3, 11.3 Hz, 1H), 2.14 (t, J = 10.4 Hz, 1H), 2.02 (br s, 1H) | 495.2 |
| 138 | 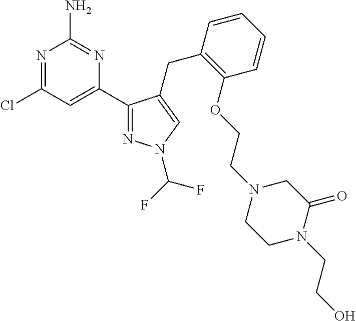 [SMILES: Nc1nc(Cl)cc(-c2nn(C(F)F)cc2Cc2ccccc2OCCN2CCN(CCO)C(=O)C2)n1] | 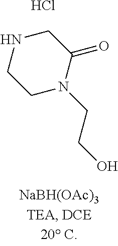 | (CDCl3) δ 7.43 (s, 1H), 7.32 (s, 0.28H), 7.29 (s, 1H), 7.28-7.22 (m, 1H), 7.17 (s, 0.52H), 7.15 (s, 1H), 7.02 (s, 0.26H), 6.97-6.86 (m, 2H), 5.49 (br s, 2H), 4.28 (s, 2H), 4.13 (t, J = 4.9 Hz, 2H), 3.83 (br d, J = 4.2 Hz, 2H), 3.57-3.52 (m, 2H), 3.37-3.34 (m, 2H), 3.33 (s, 2H), 3.16 (br s, 1H), 2.84 (t, J = 4.9 Hz, 2H), 2.76 (t, J = 5.4 Hz, 2H) | 522.2 |
| 139 | 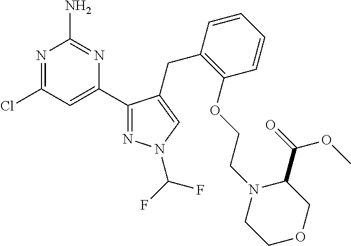 [SMILES: COC(=O)[C@H]1COCCN1CCOc1ccccc1Cc1cn(C(F)F)nc1-c1cc(Cl)nc(N)n1] | 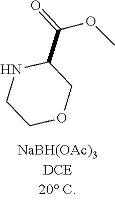 | (CDCl3) δ 7.51-7.47 (m, 1H), 7.33-7.27 (m, 1H), 7.21 (dt, J = 1.5, 7.8 Hz, 1H), 7.16 (s, 0.5H), 7.13- 7.08 (m, 1H), 7.01 (s, 0.2H), 6.92-6.86 (m, 2H), 5.36-5.26 (m, 2H), 4.33- 4.16 (m, 2H), 4.16-4.06 (m, 2H), 3.90-3.83 (m, 1H), 3.80-3.67 (m, 5H), 3.66-3.58 (m, 1H), 3.43- 3.37 (m, 1H), 3.25-3.15 (m, 1H), 3.07-2.88 (m, 2H), 2.54-2.45 (m, 1H) | 523.2 |
| 140 | 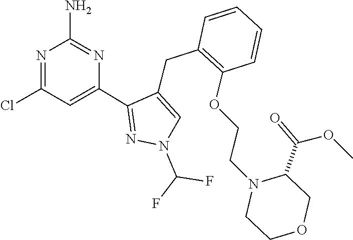 [SMILES: COC(=O)[C@@H]1COCCN1CCOc1ccccc1Cc1cn(C(F)F)nc1-c1cc(Cl)nc(N)n1] | 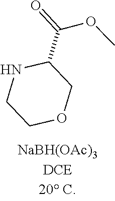 | (CDCl3) δ 7.51-7.46 (m, 1H), 7.33-7.29 (m, 1H), 7.25-7.18 (m, 1H), 7.16 (s, 0.5H), 7.13-7.08 (m, 1H), 7.02-7.00 (m, 0.3H), 6.92-6.86 (m, 2H), 5.30 (br s, 2H), 4.32-4.16 (m, 2H), 4.16-4.06 (m, 2H), 3.91-3.83 (m, 1H), 3.80- 3.67 (m, 5H), 3.66-3.58 (m, 1H), 3.40 (dd, J = 3.7, 5.1 Hz, 1H), 3.24- 3.16 (m, 1H), 3.06-2.91 (m, 2H), 2.55-2.46 (m, 1H) | 523.2 |
| 141 | 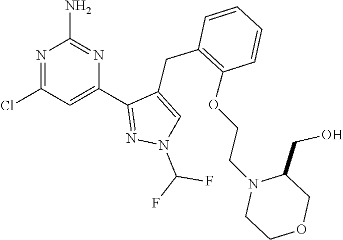 [SMILES: Nc1nc(Cl)cc(-c2nn(C(F)F)cc2Cc2ccccc2OCCN2CCOC[C@@H]2CO)n1] | 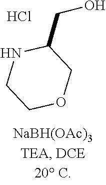 | (CDCl3) δ 7.43 (s, 1H), 7.32-7.27 (m, 1H), 7.26- 7.19 (m, 1H), 7.16 (s, 0.5H), 7.08 (dd, J = 1.6, 7.7 Hz, 1H), 7.02-7.00 (m, 0.3H), 6.94-6.88 (m, 2H), 5.56-5.35 (m, 2H), 4.35-4.20 (m, 2H), 4.18- 4.10 (m, 1H), 4.09-4.02 (m, 1H), 3.85 (dd, J = 4.3, 11.6 Hz, 1H), 3.80- 3.70 (m, 2H), 3.63-3.46 (m, 2H), 3.41 (br d, J = 11.6 Hz, 1H), 3.25 (ddd, J = 3.8, 8.0, 14.2 Hz, 1H), | 495.2 |
| | | | 2.90 (td, J = 2.7, 11.8 Hz, | |
| | | | 1H), 2.80-2.65 (m, 2H), | |
| | | | 2.59-2.47 (m, 2H) | |
| 145 | 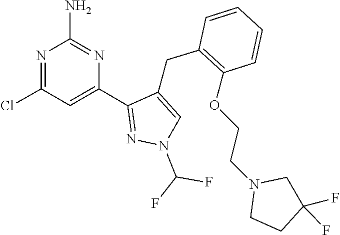 [SMILES: Nc1nc(Cl)cc(-c2nn(C(F)F)cc2Cc2ccccc2OCCN2CCC(F)(F)C2)n1] | 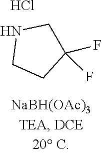 | (CDCl3) δ 7.47 (s, 1H), 7.31 (s, 1H), 7.23 (dt, J = 1.7, 7.8 Hz, 1H), 7.17- 7.12 (m, 1.4H), 7.00 (s, 0.2H), 6.94-6.86 (m, 2H), 5.23 (br s, 2H), 4.26 (s, 2H), 4.10 (t, J = 5.3 Hz, 2H), 2.93 (t, J = 13.3 Hz, 2H), 2.85 (t, J = 5.3 Hz, 2H), 2.79 (t, J = 7.0 Hz, 2H), 2.22 (tt, J = 7.2, 14.6 Hz, 2H) | 485.2 |

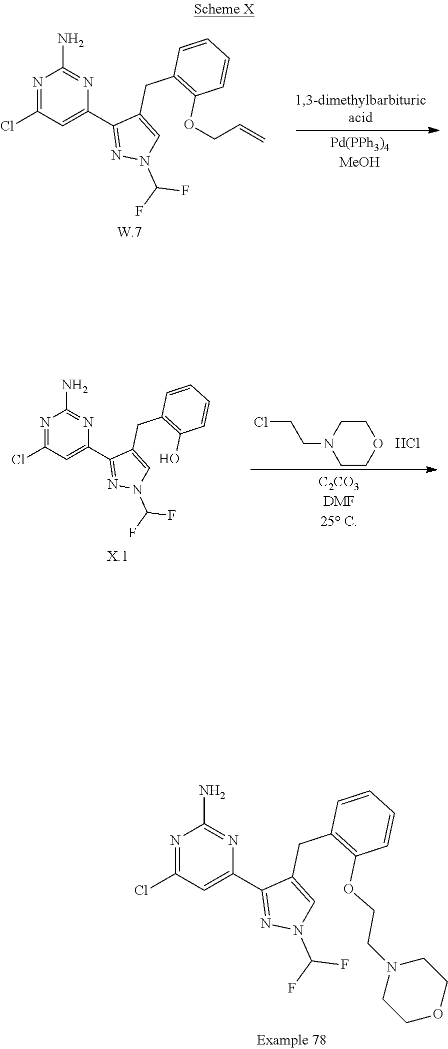 [SMILES: C=CCOc1ccccc1Cc1cn(C(F)F)nc1-c1cc(Cl)nc(N)n1 ; Nc1nc(Cl)cc(-c2nn(C(F)F)cc2Cc2ccccc2O)n1 ; ClCCN1CCOCC1 ; Cl ; Nc1nc(Cl)cc(-c2nn(C(F)F)cc2Cc2ccccc2OCCN2CCOCC2)n1]

### Step 1

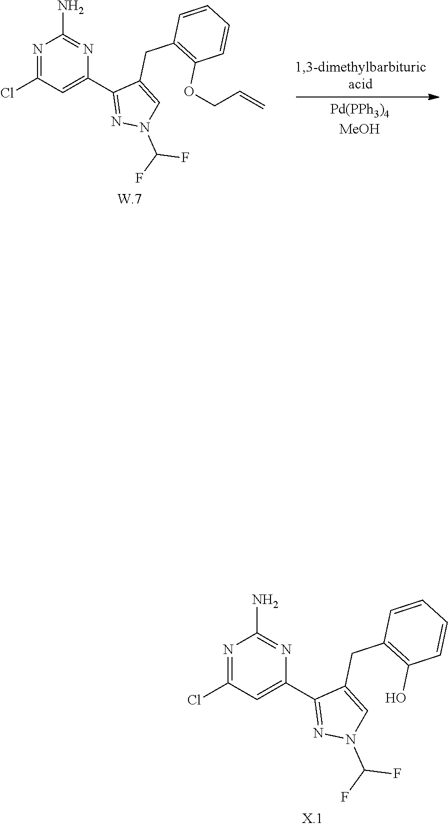 [SMILES: C=CCOc1ccccc1Cc1cn(C(F)F)nc1-c1cc(Cl)nc(N)n1 ; Nc1nc(Cl)cc(-c2nn(C(F)F)cc2Cc2ccccc2O)n1]

To a solution of 4-[4-[(2-allyloxyphenyl)methyl]-1-(difluoromethyl)pyrazol-3-yl]-6-chloro-pyrimidin-2-amine (100 mg, 0.255 mmol, 1 eq) in MeOH (4 mL) was added 1,3-dimethylhexahydropyrimidine-2,4,6-trione (80 mg, 0.51 mmol, 2 eq) and Pd(PPh₃)₄ (30 mg, 0.026 mmol, 0.1 eq). The reaction mixture was degassed and purged with N₂ (3 X). The mixture was stirred at 25° C. for 5 hr under N₂. The reaction mixture was diluted with H₂O (20 mL) and extracted with EtOAc (20 mL×3). The combined organic layers were washed with brine (20 mL), dried over Na₂SO₄, and filtered. The filtrate was concentrated under reduced pressure. The residue was purified by flash silica gel chromatography (ISCO®; 4 g SepaFlash@ Silica Flash Column, gradient of 0 to 13% ethyl acetate/petroleum ether @ 36 mL/min) which furnished 2-[[3-(2-amino-6-chloro-pyrimidin-4-yl)-1-(difluoromethyl)pyrazol-4-yl]methyl]phenol.

### Step 2

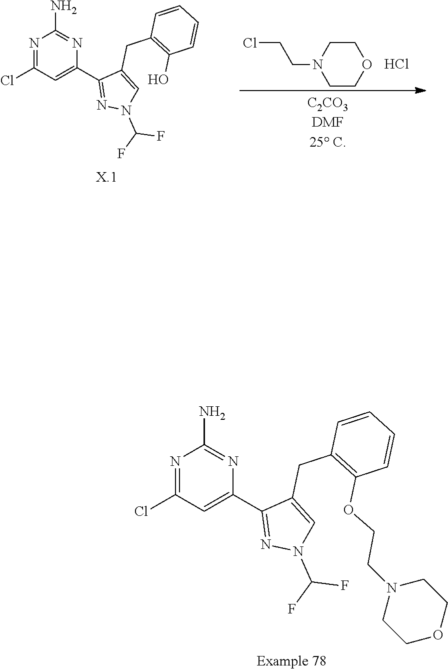 [SMILES: Nc1nc(Cl)cc(-c2nn(C(F)F)cc2Cc2ccccc2O)n1 ; ClCCN1CCOCC1 ; Cl ; Nc1nc(Cl)cc(-c2nn(C(F)F)cc2Cc2ccccc2OCCN2CCOCC2)n1]

To a mixture of 2-[[3-(2-amino-6-chloro-pyrimidin-4-yl)-1-(difluoromethyl) pyrazol-4-yl]methyl]phenol (34 mg, 0.097 mmol, 1 eq) in DMF (2 mL) was added 4-(2-chloroethyl)morpholine (36 mg, 0.19 mmol, 2 eq, HCl salt) and Cs₂CO₃ (94 mg, 0.29 mmol, 3 eq). Then the mixture was stirred at 25° C. for 40 hr under N₂. The reaction mixture was diluted with saturated aqueous NH₄Cl (20 mL) and extracted with EtOAc (10 mL×3). The combined organic layers were washed with brine (10 mL), dried over Na₂SO₄, and filtered. The filtrate was concentrated under reduced pressure. The residue was purified by preparative-HPLC (column: Welch Xtimate C18 150×25 mm, 5 m; mobile phase: [water (0.2% FA)-ACN]; B %: 30%-60%, 10 min, FA condition) which furnished 4-chloro-6-[1-(difluoromethyl)-4-[[2-(2-morpholinoethoxy)phenyl]methyl]pyrazol-3-yl]pyrimidin-2-amine Example 78.

Example 78: ¹H NMR: (400 MHz, DMSO-d₆, formic acid salt) δ 8.13 (s, 1H), 7.93-7.61 (m, 2H), 7.31-7.26 (m, 2H), 7.26-7.23 (m, 1H), 7.22-7.16 (m, 1H), 7.03-7.01 (m, 1H), 6.97 (d, J=8.4 Hz, 1H), 6.86 (t, J=7.4 Hz, 1H), 4.22 (s, 2H), 4.07-4.02 (m, 2H), 3.51-3.46 (m, 4H), 2.60 (br t, J=5.0 Hz, 2H), 2.41-2.34 (m, 4H); LCMS: (MH+) 465.2.

The examples in Table 10 were prepared in a similar fashion to that depicted in Scheme X using the appropriate conditions for Step 2.

TABLE 10

| | | | 1H NMR | |
|---|---|---|---|---|
| Ex. | Structure | Conditions | (400 MHz) | LCMS |
| 79 | 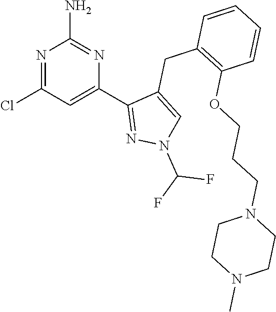 [SMILES: CN1CCN(CCCOc2ccccc2Cc2cn(C(F)F)nc2-c2cc(Cl)nc(N)n2)CC1] | 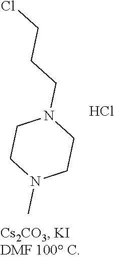 | (DMSO-d₆) δ 7.94-7.59 (m, 2H), 7.30-7.16 (m, 4H), 7.03 (s, 1H), 6.95 (d, J = 8.1 Hz, 1H), 6.86 (t, J = 7.3 Hz, 1H), 4.21 (s, 2H), 3.93 (br t, J = 6.0 Hz, 2H), 2.32-2.14 (m, 10H), 2.12 (s, 3H), 1.72 (quin, J = 6.6 Hz, 2H) | 492.2 |
| 81 | 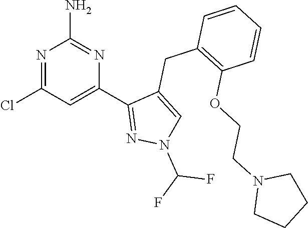 [SMILES: Nc1nc(Cl)cc(-c2nn(C(F)F)cc2Cc2ccccc2OCCN2CCCC2)n1] | 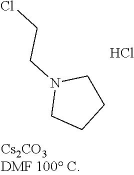 | (CDCl3, formic acid salt) δ 8.45 (s, 1H), 7.34 (s, 1H), 7.32-7.30 (m, 1H), 7.26-7.23 (m, 1H), 7.15 (s, 1H), 7.13 (s, 1H), 7.01-6.98 (m, 1H), 6.98-6.94 (m, 1H), 6.93-6.89 (m, 1H), 5.53-5.37 (m, 2H), 4.31 (t, J = 4.9 Hz, 2H), 4.26 (s, 2H), 3.24 (t, J = 4.9 Hz, | 449.0 |
| | | | 2H), 3.03 (br s, | |
| | | | 4H), 1.93-1.81 (m, | |
| | | | 4H) | |
| 85 | 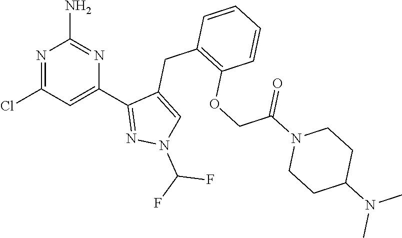 [SMILES: CN(C)C1CCN(C(=O)COc2ccccc2Cc2cn(C(F)F)nc2-c2cc(Cl)nc(N)n2)CC1] | 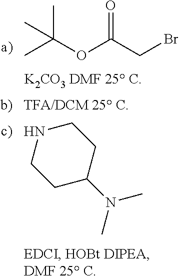 | (DMSO-d₆) δ 8.05 (s, 1H), 7.92-7.60 (m, 1H), 7.29-7.22 (m, 2H), 7.21-7.12 (m, 2H), 7.03 (s, 1H), 6.93- 6.81 (m, 2H), 4.89- 4.76 (m, 2H), 4.27 (s, 2H), 3.80 (br d, J = 13.3 Hz, 1H), 3.05-2.88 (m, 2H), 2.67 (br s, 1H), 2.34-2.30 (m, 1H), 2.13 (s, 6H), 1.77- 1.61 (m, 2H), 1.34- 1.12 (m, 2H) | 520.2 |
| 87 | 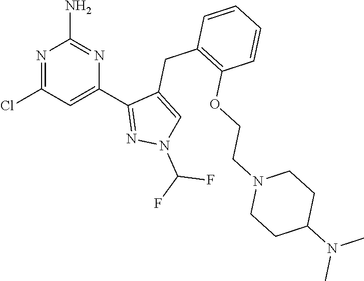 [SMILES: CN(C)C1CCN(CCOc2ccccc2Cc2cn(C(F)F)nc2-c2cc(Cl)nc(N)n2)CC1] | 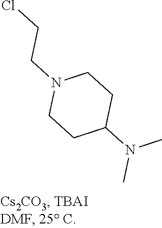 | (ACETONITRILE- d3, formic acid salt) δ 8.30 (s, 1.5H), 7.59-7.16 (m, 5H), 6.93 (d, J = 8.1 Hz, 1H), 6.88 (dt, J = 0.8, 7.4 Hz, 1H), 6.07-5.94 (m, 2H), 4.27 (s, 2H), 4.09- 4.01 (m, 2H), 3.01 (br d, J = 12.1 Hz, 2H), 2.76-2.70 (m, 1H), 2.70-2.66 (m, 2H), 2.52 (s, 6H), 2.05 (dt, J = 1.7, 12.0 Hz, 2H), 1.84 | 506.2 |
| | | | (br d, J = 12.5 Hz, | |
| | | | 2H), 1.57 (dq, J = | |
| | | | 3.8, 12.1 Hz, 2H) | |
| 95 | 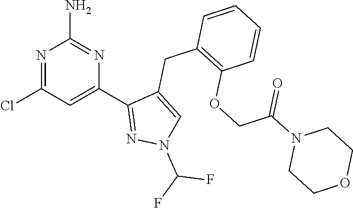 [SMILES: Nc1nc(Cl)cc(-c2nn(C(F)F)cc2Cc2ccccc2OCC(=O)N2CCOCC2)n1] | 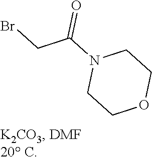 | (DMSO-d₆) δ 8.09 (s, 1H), 7.95-7.61 (m, 1H), 7.28 (br s, 2H), 7.22-7.13 (m, 2H), 7.03 (s, 1H), 6.91 (br d, J = 8.3 Hz, 1H), 6.85 (br t, J = 7.3 Hz, 1H), 4.90- 4.83 (m, 2H), 4.27 (s, 2H), 3.55 (br s, 4H), 3.44 (br d, J = 4.0 Hz, 4H) | 479.1 |
| 98 | 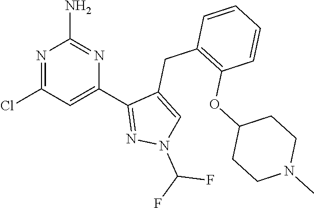 [SMILES: CN1CCC(Oc2ccccc2Cc2cn(C(F)F)nc2-c2cc(Cl)nc(N)n2)CC1] | 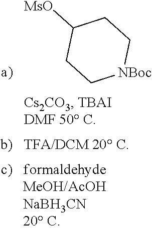 [SMILES: CC(C)(C)OC(=O)N1CCC(OS(C)(=O)=O)CC1] | (CDCl3) δ 7.43 (s, 1H), 7.30 (s, 1H), 7.24-6.85 (m, 5H), 5.19 (br s, 2H), 4.39 (br s, 1H), 4.25 (s, 2H), 2.49 (br s, 2H), 2.36-2.18 (m, 5H), 1.97 (br s, 2H), 1.81 (br d, J = 7.0 Hz, 2H) | 449.2 |
| 108 | 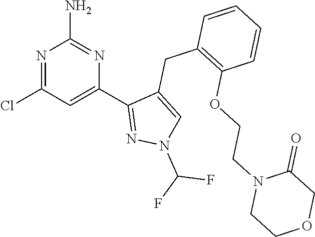 [SMILES: Nc1nc(Cl)cc(-c2nn(C(F)F)cc2Cc2ccccc2OCCN2CCOCC2=O)n1] | 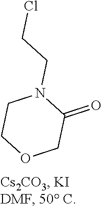 | (CDCl3) δ 7.34-7.28 (m, 2.48H), 7.24 (br s, 0.33H), 7.18-7.11 (m, 2H), 7.01-6.87 (m, 2H), 5.31-5.24 (m, 2H), 4.25-4.22 (m, 2H), 4.20 (t, J = 4.9 Hz, 2H), 4.12 (s, 2H), 3.73 (t, J = 4.9 Hz, 2H), 3.64 (t, J = 5.1 Hz, 2H), 3.36-3.29 (m, 2H) | 479.1 |
| 109 | 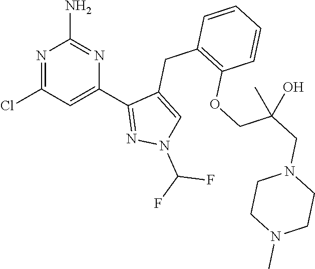 [SMILES: CN1CCN(CC(C)(O)COc2ccccc2Cc2cn(C(F)F)nc2-c2cc(Cl)nc(N)n2)CC1] | 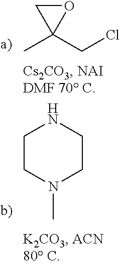 | (CDCl3) δ 7.32 (s, 0.73H), 7.22 (s, 1.39H), 7.18-7.05 (m, 2.57H), 6.92-6.84 (m, 1H), 6.83-6.77 (m, 1H), 5.20 (br s, 2H), 4.23-4.13 (m, 2H), 3.74-3.62 (m, 2H), 2.47-2.36 (m, 5H), 2.29 (br s, 4H), 2.17 (s, 3H), 2.11 (br d, J = 13.8 Hz, 1H), 1.04 (s, 3H) | 522.3 |
| 125 | 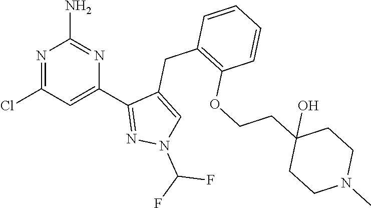 [SMILES: CN1CCC(O)(CCOc2ccccc2Cc2cn(C(F)F)nc2-c2cc(Cl)nc(N)n2)CC1] | 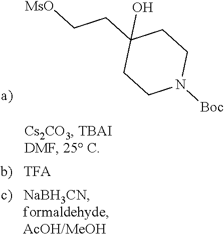 [SMILES: CC(C)(C)OC(=O)N1CCC(O)(CCOS(C)(=O)=O)CC1] | (CDCl3) δ 7.44 (s, 1H), 7.31 (s, 0.3H), 7.29 (s, 1H), 7.26-7.21 (m, 1H), 7.16 (s, 0.5H), 7.15-7.11 (m, 1H), 7.01 (s, 0.2H), 6.96-6.90 (m, 2H), 5.46 (br s, 2H), 4.28 (s, 2H), 4.23 (t, J = 5.8 Hz, 2H), 2.56 (br d, J = 10.9 Hz, 2H), 2.45 (br s, 1H), 2.29 (s, 5H), 1.96 (t, J = 5.9 Hz, | 493.2 |
| | | | 2H), 1.75-1.68 (m, | |
| | | | 2H), 1.64 (br s, 2H | |
| 130 | 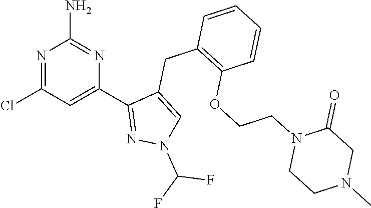 [SMILES: CN1CCN(CCOc2ccccc2Cc2cn(C(F)F)nc2-c2cc(Cl)nc(N)n2)C(=O)C1] | 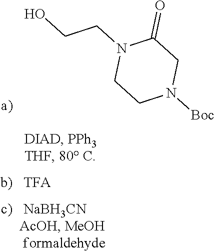 [SMILES: CC(C)(C)OC(=O)N1CCN(CCO)C(=O)C1] | (CDCl3) δ 7.37-7.29 (m, 2H), 7.25-6.86 (m, 5H), 5.29 (br s, 2H), 4.24 (s, 2H), 4.17 (t, J = 5.0 Hz, 2H), 3.71 (t, J = 5.0 Hz, 2H), 3.33 (t, J = 5.4 Hz, 2H), 3.06 (s, 2H), 2.41 (t, J = 5.4 Hz, 2H), 2.26 (s, 3H) | 492.2 |

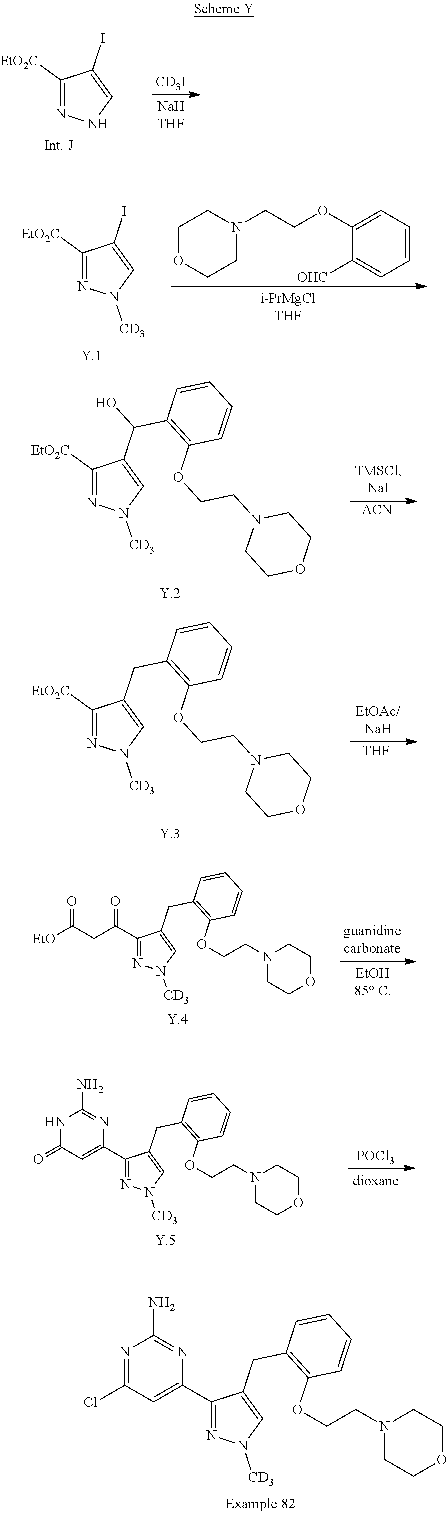

### Step 1

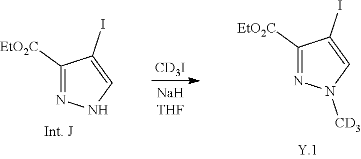

To a solution of ethyl 4-iodo-1H-pyrazole-3-carboxylate (2.00 g, 7.52 mmol, 1 eq) in THF (20 mL) was added NaH (301 mg, 7.52 mmol, 60 wt % dispersion in oil) at 0° C. The mixture was warmed to 25° C., and stirred at that temperature for 15 min. Tri-deuterio(iodo)methane (1.09 g, 7.52 mmol, 0.468 mL, 1 eq) was added dropwise at 0° C. The mixture was stirred for 12 h at 25° C. The reaction mixture was diluted with sat. aqueous NaHCO₃ (80 mL). The solution was extracted with EtOAc (40 mL×5). The organic layer was washed with brine (50 mL), dried over Na₂SO₄, and filtered. The filtrate was concentrated under the reduced pressure. The residue was purified by column chromatography (SiO₂, petroleum ether/ethyl acetate=3/1) which furnished ethyl 4-iodo-1-(trideuteriomethyl)pyrazole-3-carboxylate.

Example 82 was prepared from intermediate Y.2 using conditions similar to that depicted in Scheme B for Example 2.

Example 82: ¹H NMR: (DMSO-d6, 400 MHz) δ 7.26 (s, 1H), 7.21 (dd, J=1.6, 7.4 Hz, 1H), 7.18-7.12 (m, 1H), 7.07 (br s, 2H), 6.98 (s, 1H), 6.95 (d, J=7.8 Hz, 1H), 6.83 (t, J=7.3 Hz, 1H), 4.20 (s, 2H), 4.04 (t, J=5.6 Hz, 2H), 3.54-3.46 (m, 4H), 2.60 (t, J=5.6 Hz, 2H), 2.41-2.35 (m, 4H); LCMS: (MH+) 432.2.

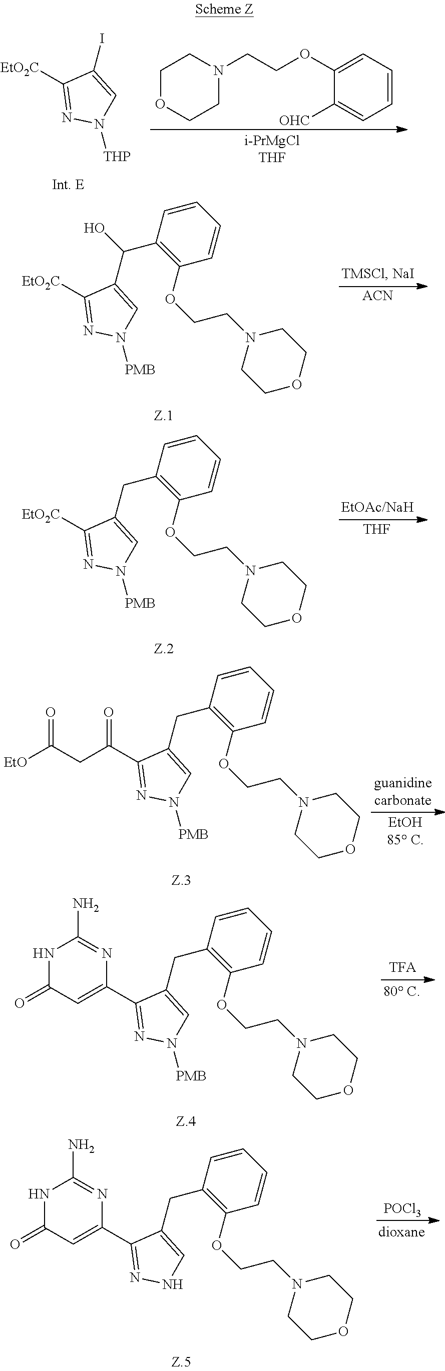

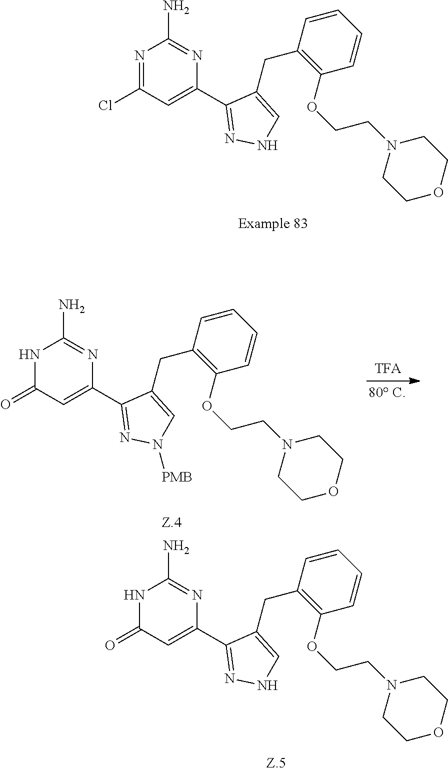

Intermediate Z.4 was prepared from Int. E using conditions similar to that depicted in Scheme D.

### Step 5

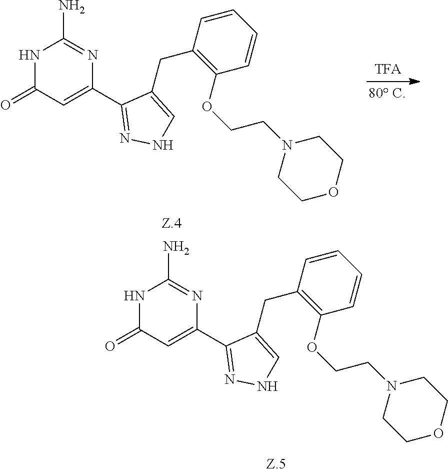 [SMILES: Nc1nc(-c2n[nH]cc2Cc2ccccc2OCCN2CCOCC2)cc(=O)[nH]1 ; Nc1nc(-c2n[nH]cc2Cc2ccccc2OCCN2CCOCC2)cc(=O)[nH]1]

2-Amino-4-[1-[(4-methoxyphenyl)methyl]-4-[[2-(2-morpholinoethoxy)phenyl]methyl]pyrazol-3-yl]-1H-pyrimidin-6-one (100 mg, 0.194 mmol, 1 eq) was dissolved in TFA (1.5 mL). The mixture was stirred at 80° C. for 18 h under N₂. The reaction mixture was poured into saturated sodium bicarbonate solution (50 mL). The mixture was extracted with ethyl acetate (20 mL×6). The organic phase was washed with brine (50 mL), dried over anhydrous Na₂SO₄, and filtered. The filtrate was concentrated which provided 2-amino-4-[4-[[2-(2-morpholinoethoxy)phenyl] methyl]-1H-pyrazol-3-yl]-1H-pyrimidin-6-one.

### Step 6

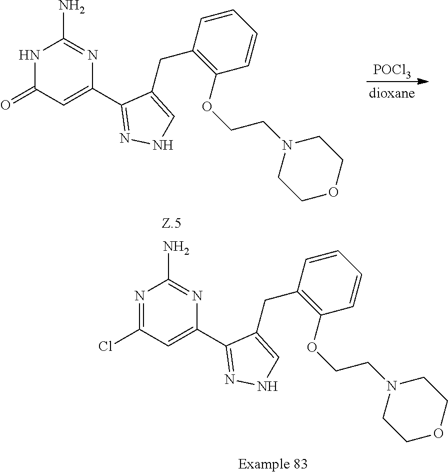 [SMILES: Nc1nc(-c2n[nH]cc2Cc2ccccc2OCCN2CCOCC2)cc(=O)[nH]1 ; "POCl3" ; Nc1nc(Cl)cc(-c2n[nH]cc2Cc2ccccc2OCCN2CCOCC2)n1]

2-Amino-4-[4-[[2-(2-morpholinoethoxy)phenyl]methyl]-1H-pyrazol-3-yl]-1H-pyrimidin-6-one (80 mg, 0.20 mmol, 1 eq) was dissolved in POCl₃ (3.30 g, 21.5 mmol, 2 mL, 107 eq). The mixture was stirred at 75° C. for 1.5 h under N₂. The reaction mixture was concentrated. The mixture was dissolved in EtOAc (30 mL) and added slowly to an aqueous, saturated sodium bicarbonate solution (150 mL). The mixture was extracted with ethyl acetate (50 mL×3). The organic phase was washed with brine (80 mL), dried over anhydrous Na₂SO₄, and filtered. The filtrate was concentrated. The residue was purified by preparative-HPLC (column: Waters Xbridge BEH C18 100×30 mm, 10 m; mobile phase: [water (10 mM NH₄HCO₃)-ACN]; B %: 25%-45%, 8 min) which furnished 4-chloro-6-[4-[[2-(2-morpholino ethoxy)phenyl]methyl]-1H-pyrazol-3-yl]pyrimidin-2-amine Example 83.

Example 83: ¹H NMR: (400 MHz, DMSO-d6) δ 7.25-7.06 (m, 4H), 6.94 (br d, J=7.8 Hz, 1H), 6.88 (t, J=7.4 Hz, 1H), 4.29-4.08 (m, 4H), 3.60 (br s, 4H), 2.72 (br s, 2H), 2.47 (br s, 4H); LCMS: (MH+) 415.2

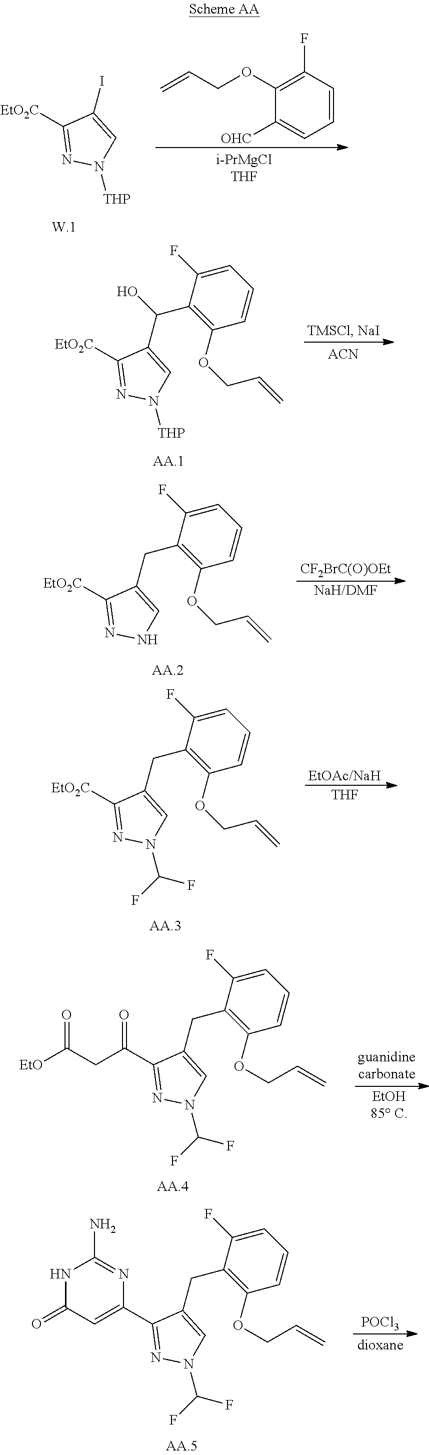

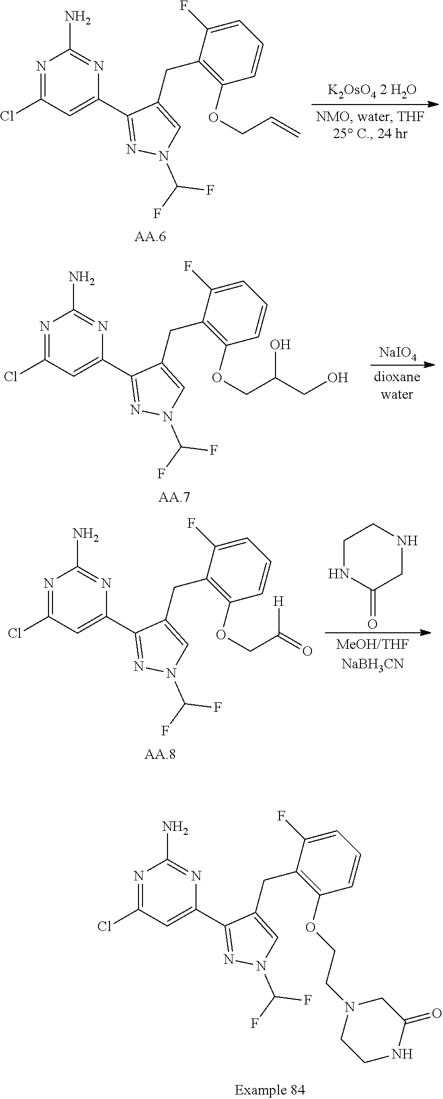 [SMILES: C=CCOc1cccc(F)c1Cc1cn(C(F)F)nc1-c1cc(Cl)nc(N)n1 ; Nc1nc(Cl)cc(-c2nn(C(F)F)cc2Cc2c(F)cccc2OCC(O)CO)n1 ; "NaIO4" ; Nc1nc(Cl)cc(-c2nn(C(F)F)cc2Cc2c(F)cccc2OCC=O)n1 ; O=C1CNCCN1 ; Nc1nc(Cl)cc(-c2nn(C(F)F)cc2Cc2c(F)cccc2OCCN2CCNC(=O)C2)n1]

Example 84 was prepared in a similar fashion to that described in Scheme W using the appropriate aldehyde in Step 1 (Scheme AA).

Example 84: ¹H NMR: (400 MHz, DMSO-d₆) δ 7.88-7.52 (m, 3H), 7.32-7.22 (m, 3H), 7.06 (s, 1H), 6.91 (d, J=8.4 Hz, 1H), 6.83 (t, J=8.8 Hz, 1H), 4.23 (s, 2H), 4.09 (t, J=5.4 Hz, 2H), 2.99-2.91 (m, 4H), 2.61 (t, J=5.4 Hz, 2H), 2.52-2.51 (m, 2H); LCMS: (MH+) 496.2.

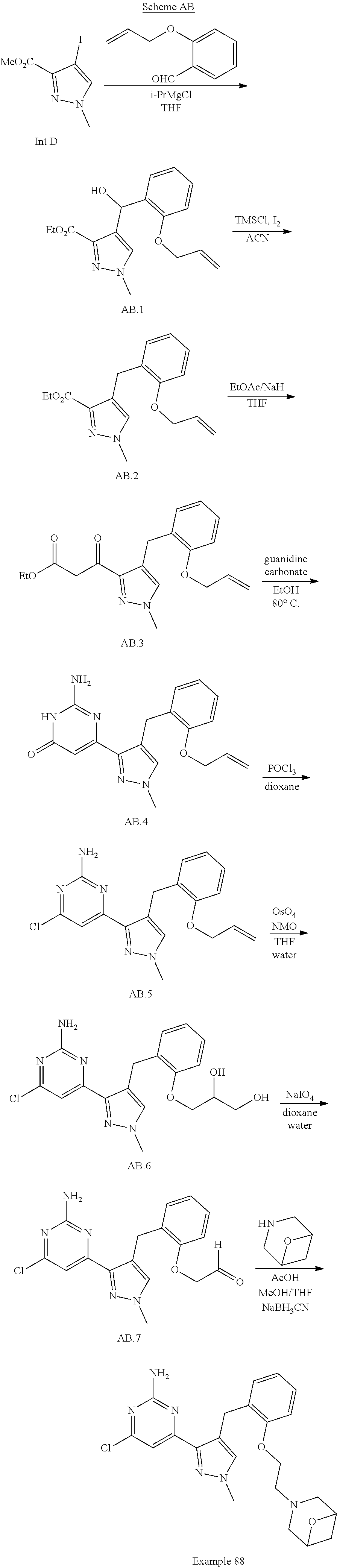

The aldehyde AB.7 was prepared in a similar fashion to that described in Scheme W for W.9.

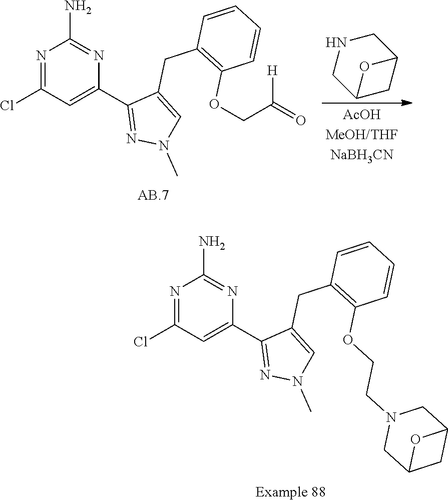 [SMILES: Cn1cc(Cc2ccccc2OCC=O)c(-c2cc(Cl)nc(N)n2)n1 ; C1NCC2CC1O2 ; Cn1cc(Cc2ccccc2OCCN2CC3CC(C2)O3)c(-c2cc(Cl)nc(N)n2)n1]

### Step 8

To a solution of 2-[2-[[3-(2-amino-6-chloro-pyrimidin-4-yl)-1-methyl-pyrazol-4-yl]methyl]phenoxy]acetaldehyde (0.11 g, 0.31 mmol, 1 eq) in MeOH (1 mL) and THF (0.5 mL) was added TEA (37 mg, 0.37 mmol, 1.2 eq) and 6-oxa-3-azabicyclo[3.1.1]heptane (50 mg, 0.37 mmol, 1.2 eq, HCl salt). The mixture was stirred at 25° C. for 2 h under N₂. Sodium cyanoborohydride (58 mg, 0.92 mmol, 3 eq) was added to the mixture. The mixture was stirred at 25° C. for another 2 h under N₂. The reaction mixture was poured into H₂O (100 mL). The mixture was extracted with ethyl acetate (30 mL×3). The organic phase was washed with brine (50 mL), dried over anhydrous Na₂SO₄, and filtered. The filtrate was concentrated. The residue was first purified by preparative-TLC (EtOAc/MeOH=20/1). The compound was further purified by preparative-HPLC (column: Waters Xbridge BEH C18 100×30 mm, m; mobile phase: [water (10 mM NH₄HCO₃)-ACN]; B %: 25%-55%, 8 min) which furnished 4-chloro-6-[1-methyl-4-[[2-[2-(6-oxa-3-azabicyclo[3.1.1] heptan-3-yl)ethoxy]phenyl]methyl]pyrazol-3-yl]pyrimidin-2-amine (14 mg) Example 88.

Example 88: ¹H NMR: (400 MHz, DMSO-d6) δ 7.23-7.13 (m, 3H), 7.06 (br s, 2H), 7.00-6.96 (m, 2H), 6.84 (t, J=7.3 Hz, 1H), 4.33 (d, J=6.0 Hz, 2H), 4.21 (s, 2H), 4.09 (t, J=5.6 Hz, 2H), 3.78 (s, 3H), 3.00 (d, J=11.3 Hz, 2H), 2.87 (t, J=5.6 Hz, 2H), 2.78 (q, J=6.4 Hz, 1H), 2.67 (d, J=11.3 Hz, 2H), 2.09 (d, J=7.8 Hz, 1H); LCMS: (MH+) 441.2.

The examples in Table 11 were prepared in a similar fashion to that depicted in Scheme AB using the appropriate conditions in Step 8.

TABLE 11

| Ex. | Structure | Conditions | 1H NMR (400 MHz) | LCMS |
|---|---|---|---|---|
| 89 | 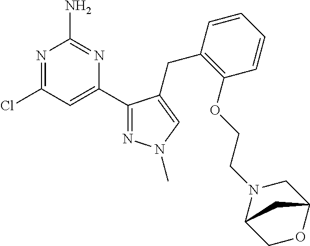 [SMILES: Cn1cc(Cc2ccccc2OCCN2C[C@H]3C[C@@H]2CO3)c(-c2cc(Cl)nc(N)n2)n1] | 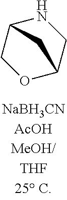 | (CDCl3, formic acid salt) δ 8.35 (s, 1H), 7.23 (s, 1H), 7.22-7.15 (m, 1H), 7.12-7.07 (m, 1H), 7.03 (s, 1H), 6.92-6.84 (m, 2H), 5.26 (br s, 2H), 4.37 (s, 1H), 4.28-4.18 (m, 2H), 4.17-4.10 (m, 2H), 4.05 (d, J = 8.1 Hz, 1H), 3.88 (s, 3H), 3.65 (s, 1H), 3.58 (dd, J = 1.6, 8.1 Hz, 1H), 3.14-3.06 (m, 2H), 3.05-2.97 (m, 1H), | 441.3 |
| | | | 2.70 (d, J = 10.4 Hz, | |
| | | | 1H), 1.88 (br s, 1H), | |
| | | | 1.73 (br d, J = 9.9 Hz, | |
| | | | 1H) | |
| 92 | 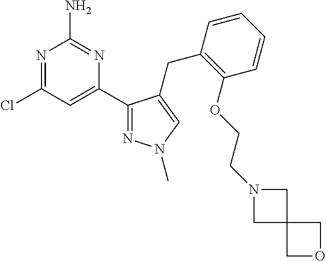 [SMILES: Cn1cc(Cc2ccccc2OCCN2CC3(COC3)C2)c(-c2cc(Cl)nc(N)n2)n1] | 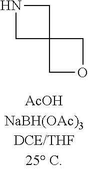 | (DMSO-d6) δ 7.29 (s, 1H), 7.19-7.10 (m, 2H), 7.08 (s, 2H), 7.00 (s, 1H), 6.89 (d, J = 8.0 Hz, 1H), 6.82 (t, J = 7.4 Hz, 1H), 4.50 (s, 4H), 4.20 (s, 2H), 3.87 (t, J = 5.1 Hz, 2H), 3.81 (s, 3H), 3.26 (s, 4H), 2.62 (br t, J = 5.1 Hz, 2H) | 441.2 |
| 93 | 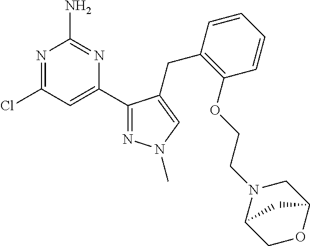 [SMILES: Cn1cc(Cc2ccccc2OCCN2C[C@@H]3C[C@H]2CO3)c(-c2cc(Cl)nc(N)n2)n1] | 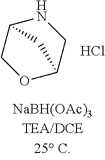 | (DMSO-d6) δ 7.32 (s, 1H), 7.22 (br t, J = 7.5 Hz, 1H), 7.16-7.09 (m, 2H), 6.96-6.87 (m, 2H), 5.32 (br s, 2H), 4.41 (br s, 1H), 4.34- 4.19 (m, 2H), 4.19- 4.10 (m, 2H), 4.07 (br d, J = 7.9 Hz, 1H), 3.93 (s, 3H), 3.68-3.55 (m, 2H), 3.12-3.03 (m, 2H), 3.03-2.95 (m, 1H), 2.71 (br d, J = 10.0 Hz, 1H), 1.87 (br d, J = 9.4 Hz, 1H), | 441.2 |
| | | | 1.75 (br d, J = 9.8 Hz, | |
| | | | 1H) | |
| 96 | 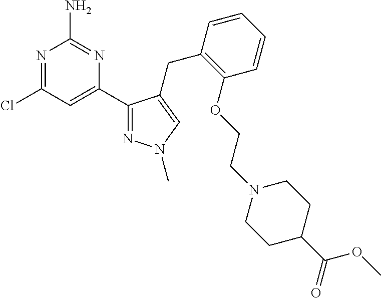 [SMILES: COC(=O)C1CCN(CCOc2ccccc2Cc2cn(C)nc2-c2cc(Cl)nc(N)n2)CC1] | 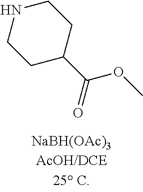 | (CDCl3) δ 7.30 (s, 1H), 7.22 (br t, J = 7.7 Hz, 1H), 7.16 (br d, J = 7.1 Hz, 1H), 7.10 (s, 1H), 6.94-6.87 (m, 2H), 5.22 (br s, 2H), 4.25 (s, 2H), 4.14 (t, J = 5.6 Hz, 2H), 3.91 (s, 3H), 3.72 (s, 3H), 2.95 (br d, J = 11.6 Hz, 2H), 2.78 (br t, J = 5.6 Hz, 2H), 2.37-2.25 (m, 1H), 2.16 (br t, J = 10.9 Hz, 2H), 1.95- 1.85 (m, 2H), 1.81- 1.71 (m, 2H) | 485.2 |
| 97 | 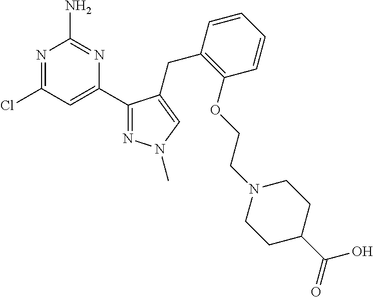 [SMILES: Cn1cc(Cc2ccccc2OCCN2CCC(C(=O)O)CC2)c(-c2cc(Cl)nc(N)n2)n1] | 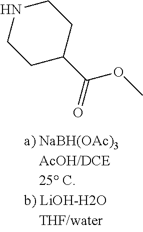 | (DMSO-d6,) δ 12.08 (br s, 1H), 7.33 (s, 1H), 7.21 (br d, J = 6.8 Hz, 1H), 7.14 (br s, 1H), 7.06 (br s, 2H), 6.98 (s, 1H), 6.94 (br d, J = 7.8 Hz, 1H), 6.87-6.78 (m, 1H), 4.21 (br s, 2H), 4.02 (br s, 2H), 3.80 (s, 3H), 2.81 (br d, J = 10.3 Hz, 2H), 2.61 (br s, 2H), 2.13 (br s, 1H), 2.06-1.94 (m, 2H), 1.70 (br s, 2H), 1.49 (br d, J = 10.6 Hz, 2H) | 471.2 |
| 119 | 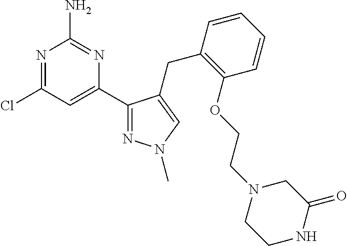 [SMILES: Cn1cc(Cc2ccccc2OCCN2CCNC(=O)C2)c(-c2cc(Cl)nc(N)n2)n1] | 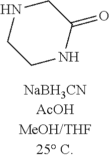 | (DMSO-d6) δ 7.75 (br s, 1H), 7.38 (s, 1H), 7.21 (dd, J = 1.4, 7.4 Hz, 1H), 7.18-7.13 (m, 1H), 7.09 (s, 2H), 6.99 (s, 1H), 6.96 (d, J = 8.0 Hz, 1H), 6.83 (t, J = 7.3 Hz, 1H), 4.23 (s, 2H), 4.08 (t, J = 5.3 Hz, 2H), 3.80 (s, 3H), 3.11-3.07 (m, 2H), 3.03 (s, 2H), 2.73 (br t, J = 5.4 Hz, 2H), 2.63-2.59 (m, 2H) | 442.2 |

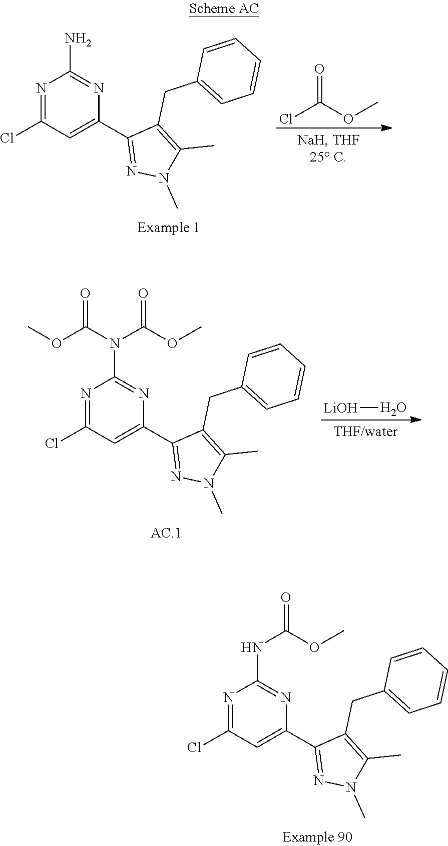

### Step 1

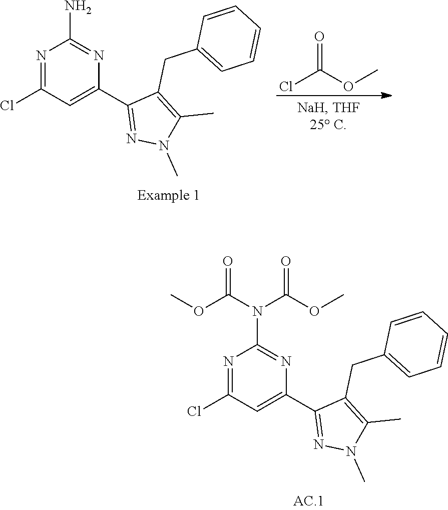

To a solution of 4-(4-benzyl-1,5-dimethyl-pyrazol-3-yl)-6-chloro-pyrimidin-2-amine (50 mg, 0.16 mmol, 1 eq) in THF (1 mL) was added NaH (255 mg, 6.37 mmol, 60 wt % dispersion in oil) at 0° C. The mixture was stirred at 0° C. for 5 min. Methyl chloroformate (151 mg, 1.59 mmol, 0.123 mL, 10 eq) was added to the mixture. The mixture was stirred at 25° C. for 12 h under N₂. The reaction mixture was diluted with sat. aqueous NH₄Cl solution (30 mL). The mixture was extracted with EtOAc (20 mL×3). The organic layer was washed with brine (30 mL), dried over Na₂SO₄, and filtered. The filtrate was concentrated under reduced pressure which furnished methyl N-[4-(4-benzyl-1,5-dimethyl-pyrazol-3-yl)-6-chloro-pyrimidin-2-yl]-N-methoxycarbonyl-carbamate. The material was used in the next step without further purification.

### Step 2

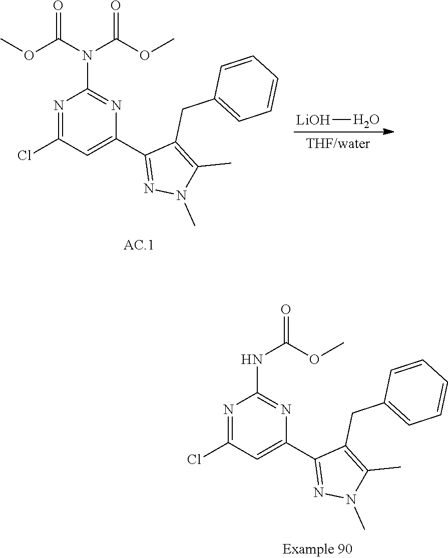

To a mixture of methyl N-[4-(4-benzyl-1,5-dimethyl-pyrazol-3-yl)-6-chloro-pyrimidin-2-yl]-N-methoxycarbonyl-carbamate (115 mg, 0.268 mmol, 1 eq) in THF (1 mL) and H₂O (0.2 mL) was added LiOH·H₂O (34 mg, 0.80 mmol, 3 eq). Then the mixture was stirred at 25° C. for 1 h under N₂. The reaction mixture was diluted with water (30 mL) and extracted with EtOAc (20 mL×3). The organic layer was washed with brine (30 mL), dried over Na₂SO₄, and filtered. The filtrate was concentrated under reduced pressure. The residue was purified by preparative-HPLC (column: Waters Xbridge BEH C18 100×25 mm, 5 m; mobile phase: [water (10 mM NH₄HCO₃)-ACN]; B %: 35%-65%, 10 min) which furnished methyl N-[4-(4-benzyl-1,5-dimethyl-pyrazol-3-yl)-6-chloro-pyrimidin-2-yl]carbamate Example 90.

Example 90: ¹H NMR: (400 MHz, DMSO-d₆) δ 7.51 (s, 1H), 7.19-7.11 (m, 4H), 7.09-7.04 (m, 1H), 4.47 (s, 2H), 3.81 (s, 3H), 3.64 (s, 3H), 2.20 (s, 3H); LCMS: (MH+) 372.1.

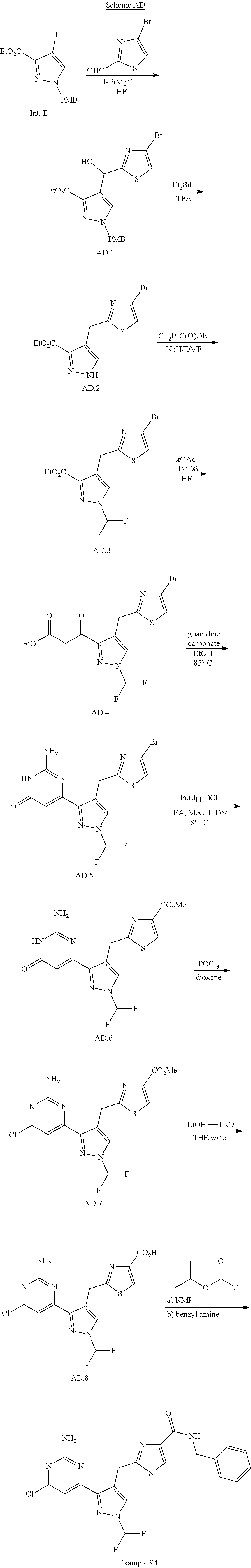

Intermediate AD.1 was prepared from Int E using conditions similar to that depicted in Step 1 of Scheme E.

### Step 2

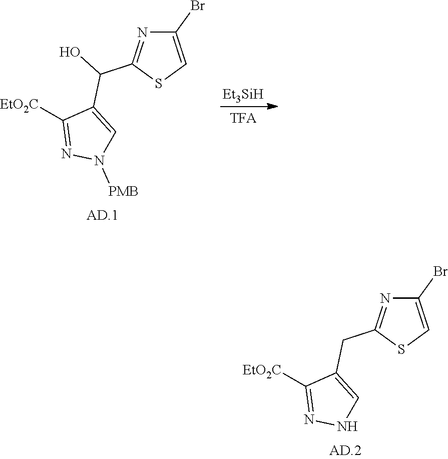

To a solution of ethyl 4-[(4-bromothiazol-2-yl)-hydroxy-methyl]-1-[(4-methoxyphenyl) methyl]pyrazole-3-carboxylate (6.00 g, 13.3 mmol, 1 eq) in TFA (60 mL) at 0° C. was added triethylsilane (4.63 g, 39.8 mmol, 6.36 mL, 3 eq). The solution was stirred at 25° C. for 12 h under N₂. The mixture was stirred at 60° C. for another 2 h under N₂. The reaction was concentrated under reduced pressure to remove the TFA. The reaction mixture was diluted with H₂O (150 mL) and extracted with EtOAc (100 mL×3). The organic layer was washed with brine (150 mL), dried over Na₂SO₄, and filtered. The filtrate was concentrated under reduced pressure. The residue was purified by flash chromatography (ISCO®; 80 g SepaFlash@ Silica Flash Column, gradient of 20 to 50% EtOAc/petroleum ether @ 100 mL/min) which furnished ethyl 4-[(4-bromothiazol-2-yl)methyl]-1H-pyrazole-3-carboxylate.

Intermediate AD.2 was converted into AD.5 using conditions similar that depicted in Scheme E. Intermediate AD.5 was converted into AD.6 using conditions outlined in Step 5 of Scheme M. Intermediate AD.6 was converted into AD.7 using conditions previously outlined in Scheme E.

### Step 8

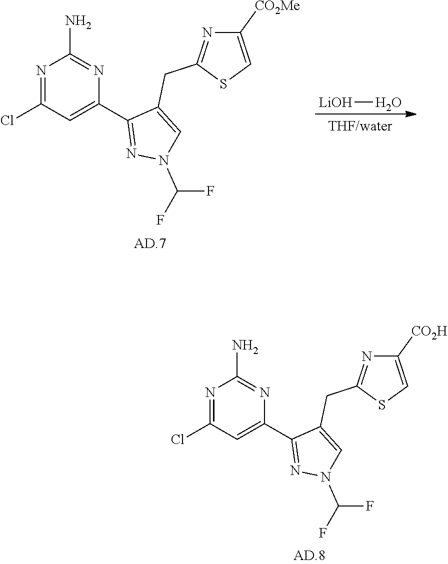

To a mixture of methyl 2-[[3-(2-amino-6-chloro-pyrimidin-4-yl)-1-(difluoromethyl)pyrazol-4-yl]methyl]thiazole-4-carboxylate (90 mg, 0.22 mmol, 1 eq) in THF (1 mL) and H₂O (0.2 mL) was added LiOH·H₂O (47 mg, 1.12 mmol, 5 eq). The mixture was stirred at 25° C. for 1 h under N₂. The reaction mixture was diluted with water, and the mixture was adjusted to pH=6 by addition of aqueous HCl solution (1 M). The mixture was extracted with EtOAc (10 mL×3). The organic layer was washed with brine (20 mL), dried over Na₂SO₄, and filtered. The filtrate was concentrated which furnished 2-[[3-(2-amino-6-chloro-pyrimidin-4-yl)-1-(difluoromethyl)pyrazol-4-yl]methyl]thiazole-4-carboxylic acid. The acid was used directly in the next step without further purification.

### Step 9

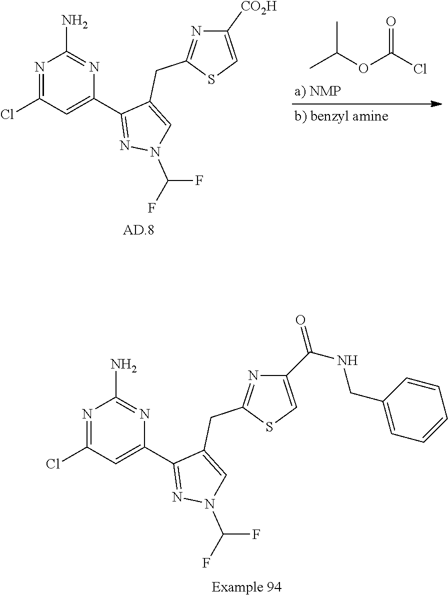

To a solution of 2-[[3-(2-amino-6-chloro-pyrimidin-4-yl)-1-(difluoromethyl)pyrazol-4-yl]methyl]thiazole-4-carboxylic acid (60 mg, 0.16 mmol, 1 eq), TEA (31 mg, 0.31 mmol, 2 eq), and NMP (0.5 mL) in DCM (0.5 mL) was added isopropyl chloroformate (29 mg, 0.23 mmol, 1.5 eq) at 0° C. dropwise. After stirring at 0° C. for 0.5 h. benzylamine (25 mg, 0.23 mmol, 1.5 eq) was added into the mixture at 0° C. The mixture was stirred at 0° C. for 10 min. The reaction mixture was diluted with water (30 mL). Then the mixture was extracted with EtOAc (20 mL×3). The organic layer was washed with brine (30 mL), dried over Na₂SO₄, and filtered. The filtrate was concentrated under reduced pressure. The residue was purified by preparative-HPLC (column: Waters Xbridge BEH C18 100×30 mm, 10 μm; mobile phase: [water (10 mM NH₄HCO₃)-ACN]; B %: 35%-65%, 8 min) which furnished 2-[[3-(2-amino-6-chloro-pyrimidin-4-yl)-1-(difluoromethyl)pyrazol-4-yl]methyl]-N-benzyl-thiazole-4-carboxamide Example 94.

Example 94: ¹H NMR: (400 MHz, CD3OD) δ 8.18 (s, 1H), 8.02 (s, 1H), 7.70-7.38 (m, 1H), 7.36-7.29 (m, 4H), 7.29-7.23 (m, 2H), 4.77 (s, 2H), 4.57 (s, 2H); LCMS: (MH+) 476.1.

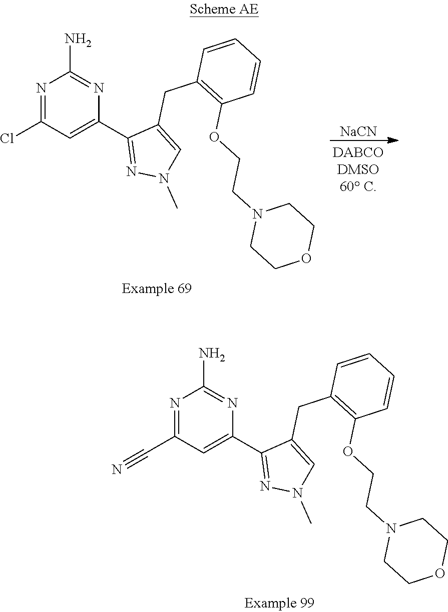 [SMILES: Cn1cc(Cc2ccccc2OCCN2CCOCC2)c(-c2cc(Cl)nc(N)n2)n1 ; "NaCN" ; Cn1cc(Cc2ccccc2OCCN2CCOCC2)c(-c2cc(C#N)nc(N)n2)n1]

4-chloro-6-[1-methyl-4-[[2-(2-morpholinoethoxy)phenyl]methyl]pyrazol-3-yl]pyrimidin-2-amine (150 mg, 0.350 mmol, 1 eq) in DMSO (2 mL) was added NaCN (21 mg, 0.42 mmol, 1.2 eq) and DABCO (47 mg, 0.42 mmol, 1.2 eq). The mixture was stirred at 60° C. for 12 h. The reaction mixture was diluted with saturated aqueous Na₂CO₃ (60 mL). The solution was extracted with EtOAc (30 mL×6). The organic layer was washed with brine (50 mL), dried over Na₂SO₄, and filtered. The filtrate was concentrated under the reduced pressure to remove the solvent. The residue was purified by preparative-HPLC (column: Waters Xbridge BEH C18 100×25 mm, 5 m; mobile phase: [water (10 mM NH₄HCO₃)-ACN]; B %: 25%-60%, 10 min) to afford 2-amino-6-[1-methyl-4-[[2-(2-morpholinoethoxy)phenyl]methyl]pyrazol-3-yl]pyrimidine-4-carbonitrile Example 99.

Example 99: ¹H NMR: (CDCl3, 400 MHz) δ 7.56 (s, 1H), 7.19 (dt, J=1.7, 7.8 Hz, 1H), 7.13-7.06 (m, 2H), 6.92-6.85 (m, 2H), 5.30 (br s, 2H), 4.23 (s, 2H), 4.14 (t, J=5.6 Hz, 2H), 3.90 (s, 3H), 3.71-3.63 (m, 4H), 2.77 (t, J=5.6 Hz, 2H), 2.62-2.48 (m, 4H); LCMS: (MH+) 420.3.

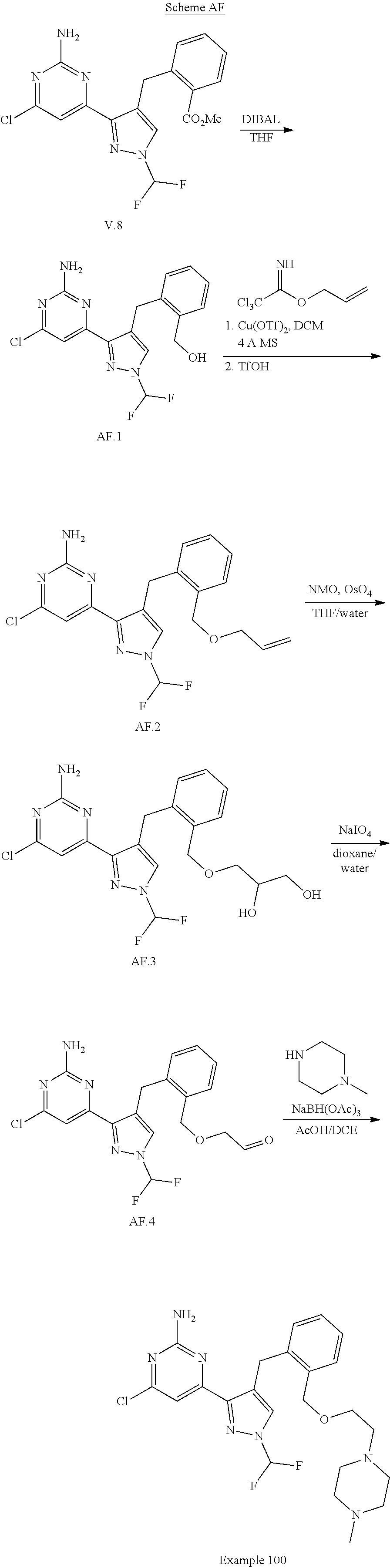

### Step 1

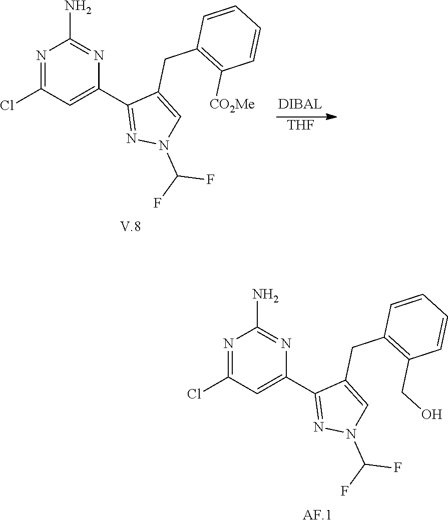 [SMILES: COC(=O)c1ccccc1Cc1cn(C(F)F)nc1-c1cc(Cl)nc(N)n1 ; Nc1nc(Cl)cc(-c2nn(C(F)F)cc2Cc2ccccc2CO)n1 ; "THF"]

To a solution of methyl 2-[[3-(2-amino-6-chloro-pyrimidin-4-yl)-1-(difluoromethyl) pyrazol-4-yl]methyl]benzoate (0.450 g, 1.14 mmol, 1 eq) in THF (5 mL) was added DIBALH (1 M, 5.71 mL, 5 eq) dropwise at −70° C. The mixture was stirred at −70° C. for 1.5 h under N₂. The mixture was warmed to 0° C. and stirred at that temperature for another 2 h under N₂. The reaction mixture was poured into saturated ammonium chloride solution (150 mL). The mixture was adjusted to pH=4-5 by addition of aqueous 1N HCl. The mixture was extracted with ethyl acetate (50 mL×3). The organic phase was washed with brine (80 mL), dried over anhydrous Na₂SO₄, and filtered. The filtrate was concentrated under reduced pressure. The residue was purified by flash chromatography (ISCO®; 4 g SepaFlash® Silica Flash Column, gradient elution of 0 to 25% ethyl acetate/petroleum ether @ 70 mL/min) to give [2-[[3-(2-amino-6-chloro-pyrimidin-4-yl)-1-(difluoromethyl)pyrazol-4-yl]methyl]phenyl]methanol.

### Step 2

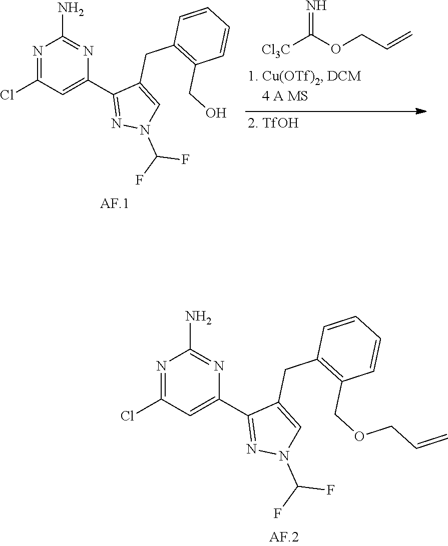

To a solution of [2-[[3-(2-amino-6-chloro-pyrimidin-4-yl)-1-(difluoromethyl)pyrazol-4-yl]methyl]phenyl]methanol (50 mg, 0.14 mmol, 1 eq) in DCM (2 mL) was added allyl 2,2,2-trichloroethanimidate (30 mg, 0.15 mmol, 1.1 eq) and 4A MS (50 mg) at 0° C. The mixture was stirred at 0° C. for 10 min under N₂. Bis-trifluoromethylsulfonyloxy) copper (59 mg, 0.16 mmol, 1.2 eq) was added to the mixture at 0° C. The mixture was stirred at 20° C. for another 50 min under N₂. Trifluoromethanesulfonic acid (205 mg, 1.37 mmol, 0.121 mL, 10 eq) was added to the mixture at 0° C. The mixture was stirred at 20° C. for 5 h under N₂. The reaction mixture was poured into saturated, aqueous sodium bicarbonate solution (50 mL). The mixture was extracted with ethyl acetate (20 mL×3). The organic phase was washed with brine (30 mL), dried over anhydrous Na₂SO₄, and filtered. The filtrate was concentrated under reduced pressure. The residue was purified by preparative-TLC (SiO₂, petroleum ether/EtOAc=2/1) which furnished 4-[4-[[2-(allyloxymethyl)phenyl]methyl]-1-(difluoromethyl)pyrazol-3-yl]-6-chloro-pyrimidin-2-amine.

Example 100 was prepared from intermediate AF.2 using conditions similar to those depicted in Scheme AB (Steps 6-8).

Example 100: ¹H NMR: (400 MHz, CDCl3, formic acid salt) δ 8.44 (s, 1H), 7.38-7.34 (m, 1H), 7.33-7.29 (m, 2H), 7.29-7.27 (m, 1H), 7.27-7.24 (m, 1H), 7.17-7.11 (m, 2H), 7.00 (s, 1H), 5.48 (br s, 2H), 4.51 (s, 2H), 4.34 (s, 2H), 3.61 (t, J=5.4 Hz, 2H), 3.45 (br s, 4H), 2.83-2.73 (m, 4H), 2.66 (t, J=5.4 Hz, 2H), 2.46 (s, 3H); LCMS: (MH+) 492.3.

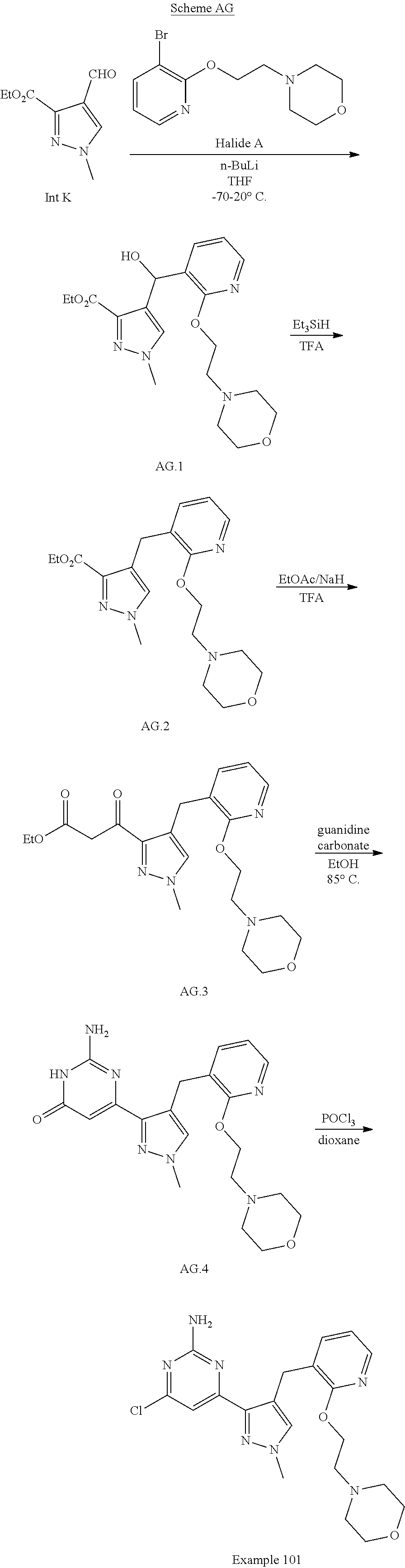

### Step 1

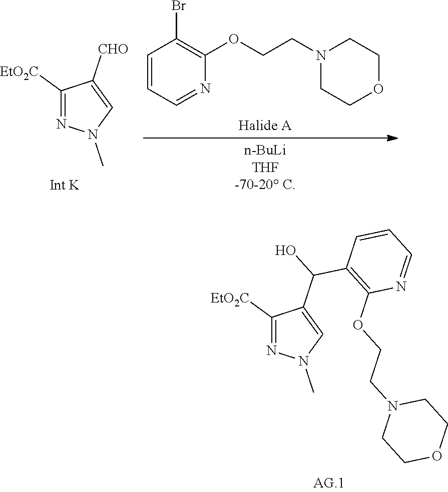

To a solution of 4-[2-[(3-bromo-2-pyridyl)oxy]ethyl]morpholine (3.00 g, 10.5 mmol, 1 eq) in THF (30 mL) was added n-BuLi (2.5 M, 8.36 mL, 2 eq) at −70° C. The mixture was stirred at −70° C. for 0.5 h under N₂. Ethyl 4-formyl-1-methyl-pyrazole-3-carboxylate (1.90 g, 10.5 mmol, 1 eq) in THF (15 mL) was added to the mixture at −70° C. The mixture was stirred at 20° C. for 12 h under N₂. The reaction mixture was poured into saturated, aqueous ammonium chloride solution (150 mL). The mixture was extracted with ethyl acetate (50 mL×3). The organic phase was washed with brine (80 mL), dried over anhydrous Na₂SO₄, and filtered. The filtrate was concentrated under reduced pressure. The residue was purified by flash chromatography (ISCO®; 40 g SepaFlash@ Silica Flash Column, gradient elution of 0 to 100% ethyl acetate/petroleum ether @ 100 mL/min, added 5% MeOH) which provided ethyl 4-[hydroxyl-[2-(2-morpholinoethoxy)-3-pyridyl]methyl]-1-methyl-pyrazole-3-carboxylate.

Example 101 was prepared from intermediate AG.1 using conditions similar to those depicted in Scheme B (Steps 2-5).

Example 101: ¹H NMR: (400 MHz, DMSO-d₆) δ 7.96 (dd, J=1.8, 5.1 Hz, 1H), 7.59 (dd, J=1.5, 7.3 Hz, 1H), 7.40 (s, 1H), 7.07 (br s, 2H), 6.97 (s, 1H), 6.85 (dd, J=5.1, 7.1 Hz, 1H), 4.33 (t, J=5.6 Hz, 2H), 4.16 (s, 2H), 3.80 (s, 3H), 3.51-3.47 (m, 4H), 2.60 (t, J=5.7 Hz, 2H), 2.40-2.34 (m, 4H); LCMS: (MH+) 430.2.

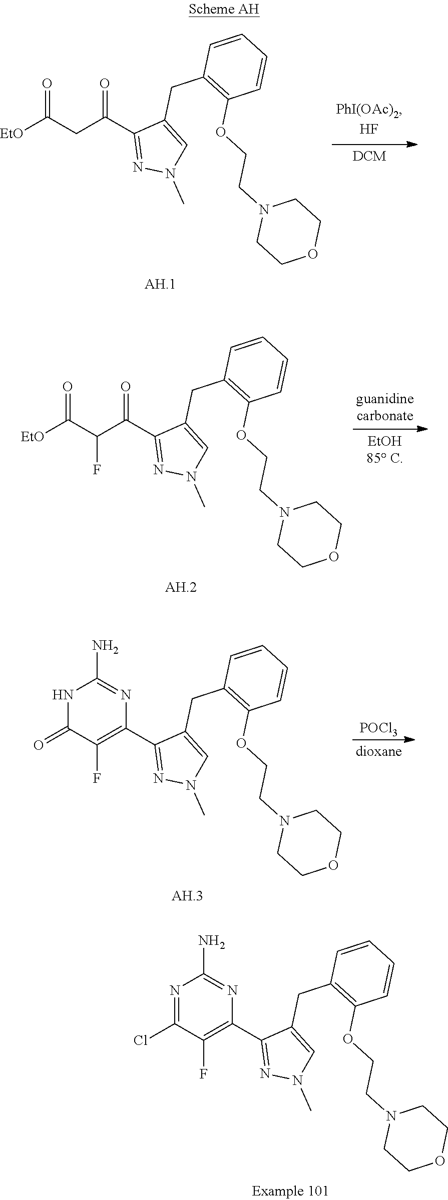 [SMILES: CCOC(=O)CC(=O)c1nn(C)cc1Cc1ccccc1OCCN1CCOCC1 ; CCOC(=O)C(F)C(=O)c1nn(C)cc1Cc1ccccc1OCCN1CCOCC1 ; Cn1cc(Cc2ccccc2OCCN2CCOCC2)c(-c2nc(N)[nH]c(=O)c2F)n1 ; "POCl3" ; Cn1cc(Cc2ccccc2OCCN2CCOCC2)c(-c2nc(N)nc(Cl)c2F)n1]

AH.1 was an intermediate used for the preparation of Example 69.

### Step 1

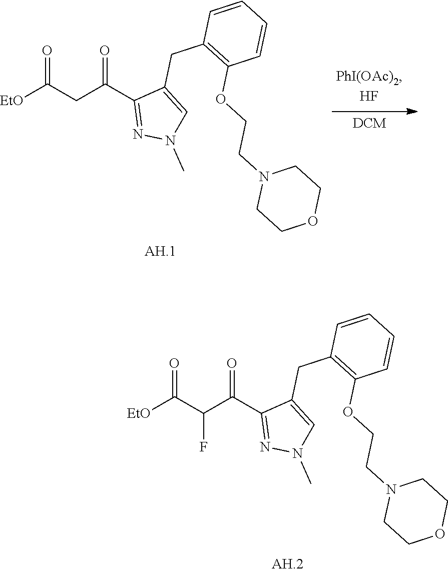 [SMILES: CCOC(=O)CC(=O)c1nn(C)cc1Cc1ccccc1OCCN1CCOCC1 ; CCOC(=O)C(F)C(=O)c1nn(C)cc1Cc1ccccc1OCCN1CCOCC1]

To a PFA test tube were added the PhI(OAc)₂ (186 mg, 0.578 mmol, 1.2 eq), HF (175 mg, 4.81 mmol, 0.160 mL, 10 eq) and DCM (8 mL). After stirring for 15 min at 20° C., ethyl 3-[1-methyl-4-[[2-(2-morpholinoethoxy)phenyl]methyl]pyrazol-3-yl]-3-oxo-propanoate (200 mg, 0.481 mmol, 1 eq) was added. The mixture was stirred at 40° C. for 12 hr. The reaction mixture was quenched with saturated, aqueous NaHCO₃ (60 mL). The mixture was extracted with EtOAc (30 mL×3). The organic layer was washed with brine (50 mL), dried over Na₂SO₄, and filtered. The filtrate was concentrated under reduced pressure. The residue was purified by flash chromatography (ISCO®; 4 g SepaFlash@ Silica Flash Column, petroleum ether/EtOAc/MeOH=10/90/5 @100 mL/min,) which furnished ethyl 2-fluoro-3-[1-methyl-4-[[2-(2-morpholinoethoxy)phenyl]methyl]pyrazol-3-yl]-3-oxo-propanoate.

Example 102 was prepared from intermediate AH.2 using conditions similar to those outlined in Scheme A (Steps 3 and 4).

Example 102: ¹H NMR: (400 MHz, CDCl3, formic acid salt) 8.23 (s, 0.12H), 7.19 (dt, J=1.6, 7.8 Hz, 1H), 7.12-7.03 (m, 2H), 6.91-6.82 (m, 2H), 5.11 (s, 2H), 4.12 (t, J=5.5 Hz, 2H), 4.04 (s, 2H), 3.92 (s, 3H), 3.73-3.66 (m, 4H), 2.79 (t, J=5.5 Hz, 2H), 2.61-2.52 (m, 4H); LCMS: (MH+) 447.2.

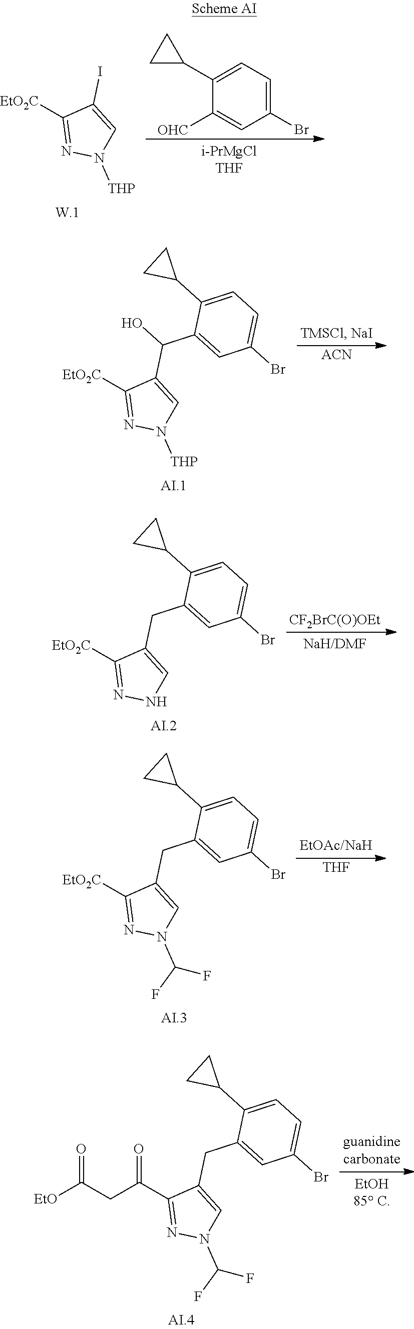

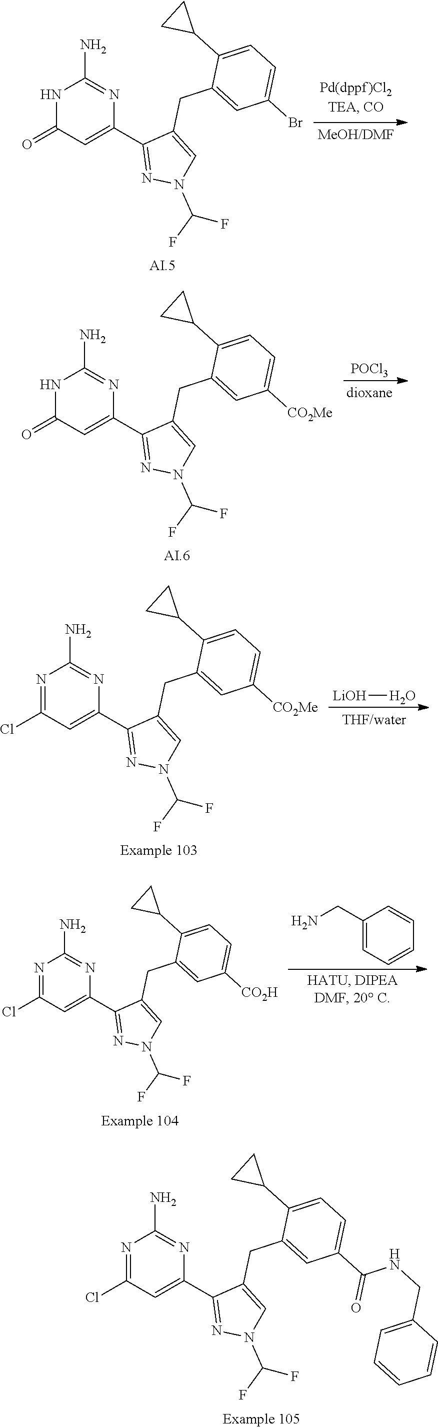

Intermediate AI.5 was prepared from W.1 using conditions similar to that depicted in Scheme W (Steps 1-6). The methyl ester AI.6 was prepared from the bromide AI.5 using conditions similar to that depicted in Scheme M (Step 5). Example 103 was prepared from AI.6 using conditions similar to that depicted in Scheme W (Step 7).

Example 103: ¹H NMR: (400 MHz, CDCl3) δ 7.91 (s, 1H), 7.87 (d, J=8.2 Hz, 1H), 7.35 (s, 1H), 7.29 (s, 0.29H), 7.28 (s, 1H), 7.14 (s, 0.5H), 7.00 (d, J=8.5 Hz, 1.25H), 5.26 (br s, 2H), 4.45 (s, 2H), 3.89 (s, 3H), 1.95-1.84 (m, 1H), 0.99-0.88 (m, 2H), 0.72 (q, J=5.2 Hz, 2H); LCMS: (MH+) 434.2.

### Step 8

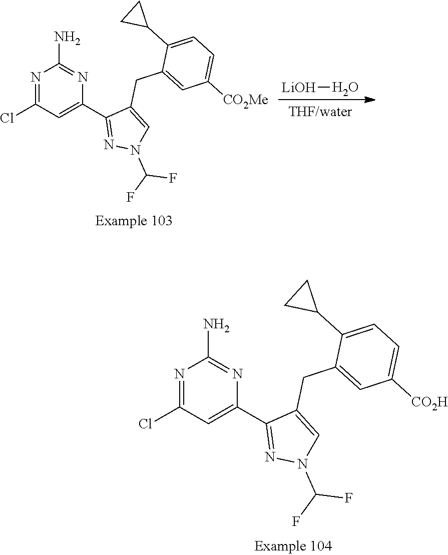

To a solution of methyl 3-[[3-(2-amino-6-chloro-pyrimidin-4-yl)-1-(difluoromethyl) pyrazol-4-yl]methyl]-4-cyclopropyl-benzoate (100 mg, 0.231 mmol, 1 eq) in THF (1.5 mL) and H₂O (0.3 mL) was added LiOH·H₂O (48 mg, 1.15 mmol, 5 eq). The mixture was stirred at 60° C. for 10 h. The reaction mixture was poured into H₂O (20 mL). The mixture was adjusted to pH=3 by addition of aqueous 4N HCl. The mixture was extracted with ethyl acetate (20 mL×8). The organic phase was washed with brine (50 mL), dried over anhydrous Na₂SO₄, and filtered. The filtrate was concentrated which furnished 3-[[3-(2-amino-6-chloro-pyrimidin-4-yl)-1-(difluoromethyl)pyrazol-4-yl]methyl]-4-cyclopropyl-benzoic acid Example 104.

Example 104: ¹H NMR: (400 MHz, DMSO-d6) δ 12.74 (bs, 1H), 7.62-7.62 (m, 3H), 7.23 (bs, 2H), 7.05 (m, 2H), 4.50 (s, 2H), 2.00 (m, 1H), 0.87 (m, 2H), 0.67 (m, 2H); LCMS: (MH+) 420.1.

### Step 9

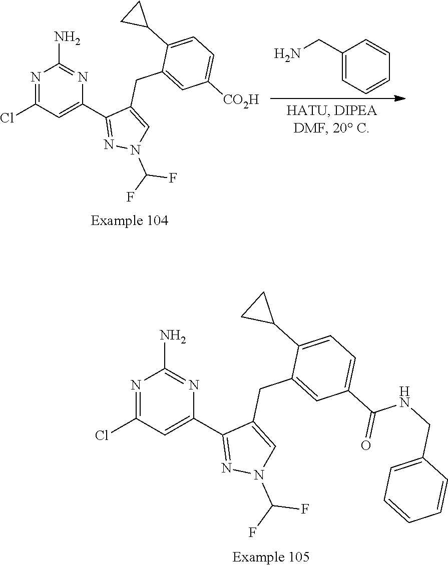 [SMILES: Nc1nc(Cl)cc(-c2nn(C(F)F)cc2Cc2cc(C(=O)O)ccc2C2CC2)n1 ; NCc1ccccc1 ; Nc1nc(Cl)cc(-c2nn(C(F)F)cc2Cc2cc(C(=O)NCc3ccccc3)ccc2C2CC2)n1]

To a solution of 3-[[3-(2-amino-6-chloro-pyrimidin-4-yl)-1-(difluoromethyl)pyrazol-4-yl]methyl]-4-cyclopropyl-benzoic acid (50 mg, 0.12 mmol, 1 eq) in DMF (2 mL) was added HATU (54 mg, 0.14 mmol, 1.2 eq) and DIPEA (31 mg, 0.24 mmol, 0.041 mL, 2 eq). The mixture was stirred at 20° C. for 15 min. Benzyl amine (26 mg, 0.24 mmol, 0.026 mL, 2 eq) was added to the mixture. The mixture was stirred at 20° C. for another 12 h under N₂. The reaction mixture was poured into H₂O (50 mL). The mixture was extracted with ethyl acetate (20 mL×3). The organic phase was washed with brine (50 mL), dried over anhydrous Na₂SO₄, and filtered. The filtrate was concentrated. The residue was purified by preparative-HPLC (column: Waters Xbridge BEH C18 100×30 mm, 10 m; mobile phase: [water (10 mM NH₄HCO₃)-ACN]; B %: 40%-70%, 8 min) which furnished 3-[[3-(2-amino-6-chloro-pyrimidin-4-yl)-1-(difluoromethyl)pyrazol-4-yl]methyl]-N-benzyl-4-cyclopropyl-benzamide Example 105.

Example 105: ¹H NMR: (400 MHz, DMSO-d6) δ 8.91 (br t, J=5.9 Hz, 1H), 7.93-7.90 (m, 0.24H), 7.80-7.75 (m, 1.5H), 7.71 (dd, J=1.7, 8.1 Hz, 1H), 7.67 (s, 1H), 7.62 (s, 0.25H), 7.36-7.19 (m, 7H), 7.07 (s, 1H), 7.02 (d, J=8.1 Hz, 1H), 4.51-4.42 (m, 4H), 2.01-1.90 (m, 1H), 0.90-0.80 (m, 2H), 0.69-0.60 (m, 2H); LCMS: (MH+) 509.2.

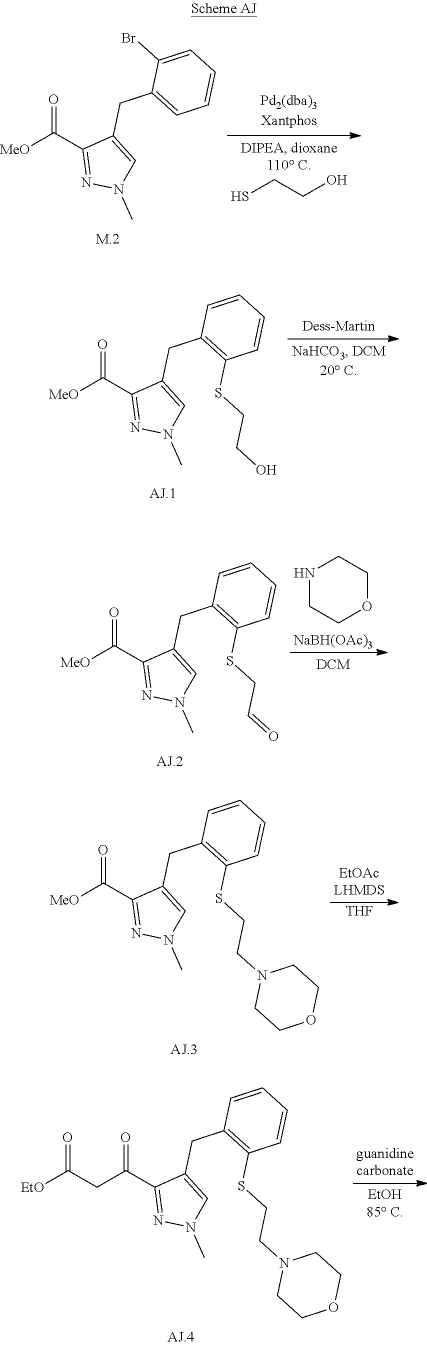

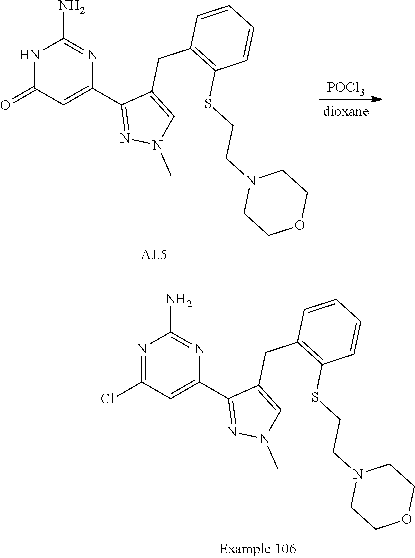 [SMILES: Cn1cc(Cc2ccccc2SCCN2CCOCC2)c(-c2cc(=O)[nH]c(N)n2)n1 ; "POCl3" ; Cn1cc(Cc2ccccc2SCCN2CCOCC2)c(-c2cc(Cl)nc(N)n2)n1]

### Step 1

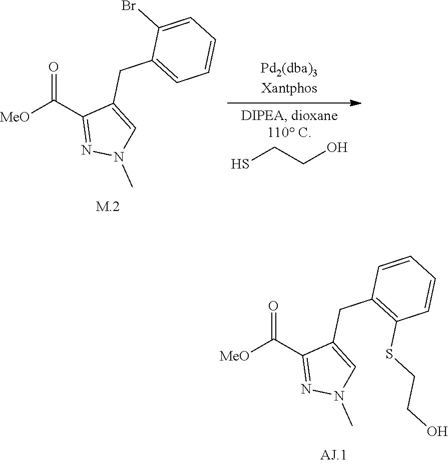

To a solution of methyl 4-[(2-bromophenyl)methyl]-1-methyl-pyrazole-3-carboxylate (5.00 g, 16.2 mmol, 1 eq) and DIPEA (2.09 g, 16.2 mmol, 2.82 mL, 1 eq) in dioxane (70 mL) was added Pd₂(dba)₃ (4.44 g, 4.85 mmol, 0.3 eq), Xantphos (5.61 g, 9.70 mmol, 0.6 eq), 2-sulfanylethanol (2.53 g, 32.4 mmol, 2.26 mL, 2 eq). The mixture was degassed and purged with N₂ (5 X). The mixture was heated at 110° C. for 12 h. The reaction mixture was filtered, and the filtrate was diluted with water. The solution was extracted with EtOAc (40 mL×3). The organic layer was washed with brine (50 mL), dried over Na₂SO₄, and filtered. The filtrate was concentrated under the reduced pressure to remove the solvent. The residue was purified by column chromatography (SiO₂, petroleum ether/ethyl acetate=0/1) to afford methyl 4-[[2-(2-hydroxyethylsulfanyl)phenyl]methyl]-1-methyl-pyrazole-3-carboxylate.

### Step 2

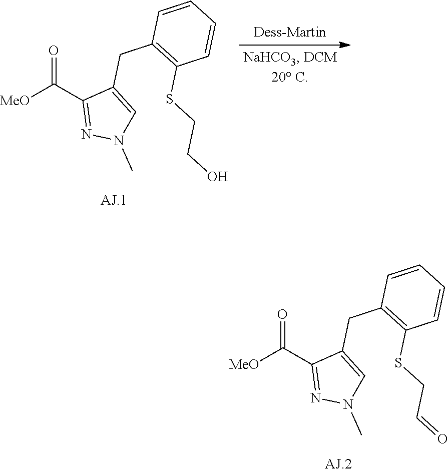 [SMILES: COC(=O)c1nn(C)cc1Cc1ccccc1SCCO ; COC(=O)c1nn(C)cc1Cc1ccccc1SCC=O]

A suspension of methyl 4-[[2-(2-hydroxyethylsulfanyl)phenyl]methyl]-1-methyl-pyrazole-3-carboxylate (0.500 g, 1.63 mmol, 1 eq) and NaHCO₃ (137 mg, 1.63 mmol, 0.063 mL, 1 eq) in DCM (5 mL) was cooled to 0° C. Dess-Martin periodinane (692 mg, 1.63 mmol, 0.505 mL, 1 eq) was added in portions at 0° C. The mixture was stirred at 20° C. for 5 h. The reaction mixture was used directly in the next step without any additional work-up.

### Step 3

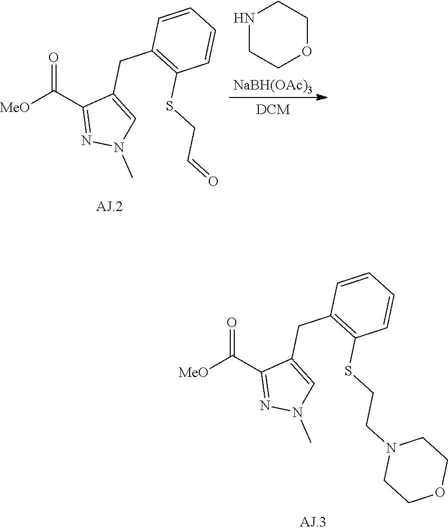

To a stirred mixture of methyl 1-methyl-4-[[2-(2-oxoethylsulfanyl)phenyl]methyl]pyrazole-3-carboxylate (500 mg, 1.64 mmol, 1 eq) was added morpholine (429 mg, 4.93 mmol, 0.434 mL, 3 eq) at 20° C. The mixture was stirred at 20° C. for 12 h. NaBH(OAc)₃ (1.04 g, 4.93 mmol, 3 eq) and DCM (3 mL) was added. The mixture was stirred at 20° C. for 3 h. The reaction mixture was diluted with water (300 mL). The solution was extracted with EtOAc (100 mL×3). The combined organic layer was washed with saturated, aqueous NaHCO₃ (100 mL), brine (150 mL), and dried over Na₂SO₄. The solution was filtered, and the filtrate was concentrated under the reduced pressure. The residue was purified by column chromatography (SiO₂, petroleum ether/ethyl acetate=2/23) to which furnished 4-[[2-(2-hydroxyethylsulfanyl)phenyl]methyl]-1-methyl-pyrazole-3-carboxylate.

Example 106 was prepared from intermediate AJ.4 using conditions similar to those depicted in Steps 3-5 of Scheme C.

Example 106: ¹H NMR: (DMSO-d6, 400 MHz) δ 7.39 (d, J=7.6 Hz, 1H), 7.25-7.18 (m, 2H), 7.15-7.09 (m, 2H), 7.04 (br s, 2H), 7.00 (s, 1H), 4.33 (s, 2H), 3.80 (s, 3H), 3.53 (t, J=4.5 Hz, 4H), 3.06-2.98 (m, 2H), 2.48-2.45 (m, 2H), 2.35 (br s, 4H); LCMS: (MH+) 445.2

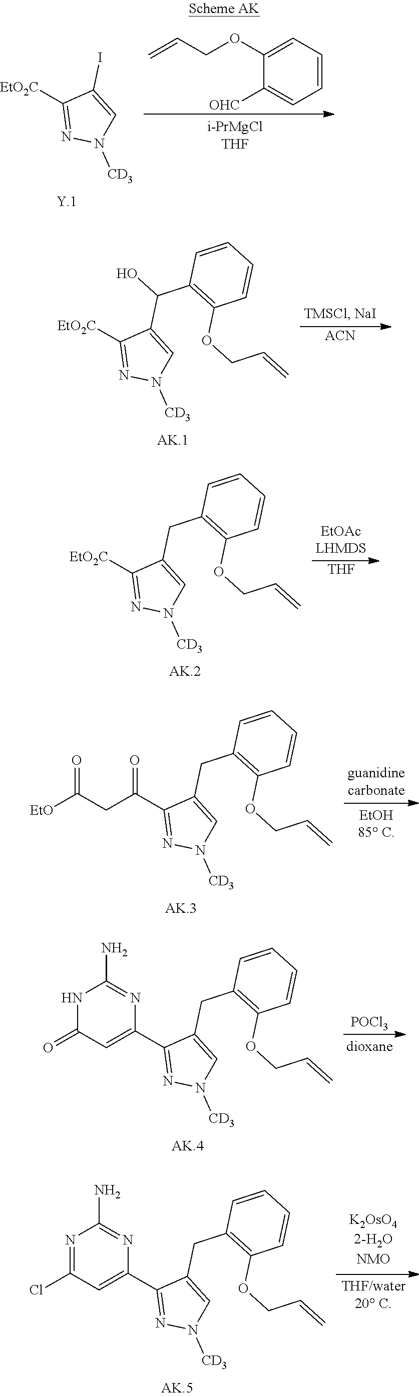

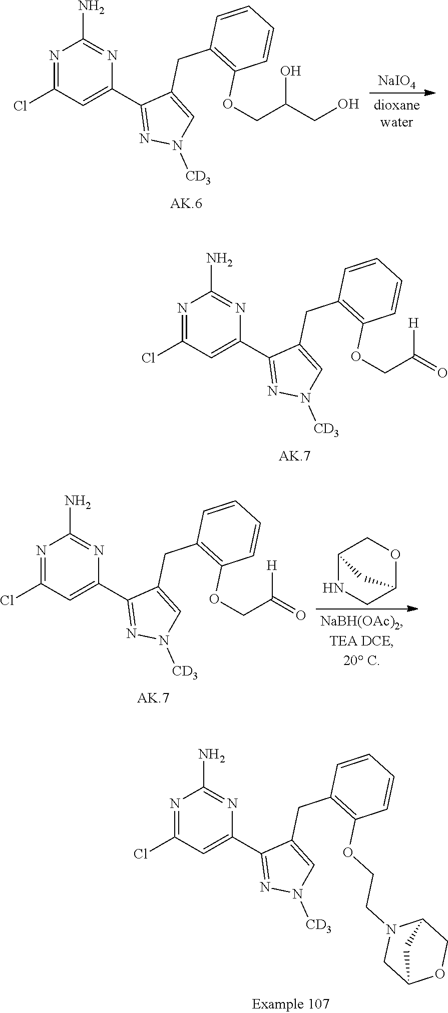

The intermediate AK.7 was prepared from Y.1 in a similar fashion to that depicted in Steps 1-7 of Scheme AB.

### Step 8

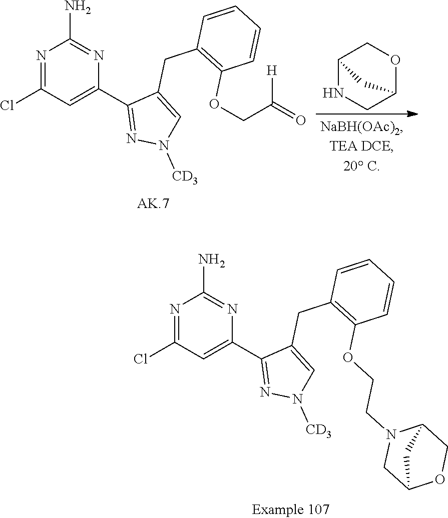

To a mixture of 2-[2-[[3-(2-amino-6-chloro-pyrimidin-4-yl)-1-(trideuteriomethyl)pyrazol-4-yl]methyl]phenoxy]acetaldehyde (80 mg, 0.22 mmol, 1 eq) and (1R,4R)-2-oxa-5-azabicyclo[2.2.1]heptane (30 mg, 0.22 mmol, 1 eq, HCl) in DCE (1 mL) was added TEA (22 mg, 0.22 mmol, 0.031 mL, 1 eq). The mixture was stirred at 20° C. for 2 h. NaBH(OAc)₃ (141 mg, 0.665 mmol, 3 eq) was added to the mixture. The mixture was stirred at 20° C. for 12 h under N₂. The reaction mixture was diluted with sat. aqueous NaHCO₃ solution (30 mL). The mixture was extracted with EtOAc (20 mL×3). The organic layer was washed with brine (30 mL), dried over Na₂SO₄, and filtered. The filtrate was concentrated under reduced pressure. The residue was purified by preparative-HPLC (column: Waters Xbridge BEH C18 100×30 mm, 10 m; mobile phase: [water (10 mM NH₄HCO₃)-ACN]; B %: 24%-54%, 8 min) which furnished 4-chloro-6-[4-[[2-[2-[(1R,4R)-2-oxa-5-azabicyclo[2.2.1]heptan-5-yl]ethoxy]phenyl]methyl]-1-(trideuteriomethyl)pyrazol-3-yl]pyrimidin-2-amine Example 107. Example 107: ¹H NMR: (400 MHz, CD3OD) δ 7.23-7.14 (m, 2H), 7.12 (s, 1H), 7.04 (s, 1H), 6.94 (d, J=8.0 Hz, 1H), 6.88 (t, J=7.3 Hz, 1H), 4.30-4.21 (m, 3H), 4.07 (t, J=5.2 Hz, 2H), 3.93 (d, J=8.1 Hz, 1H), 3.57 (s, 1H), 3.47 (dd, J=1.6, 8.0 Hz, 1H), 3.00-2.83 (m, 3H), 2.59 (d, J=10.6 Hz, 1H), 1.82-1.76 (m, 1H), 1.61 (br d, J=10.1 Hz, 1H); LCMS: (MH+) 444.2.

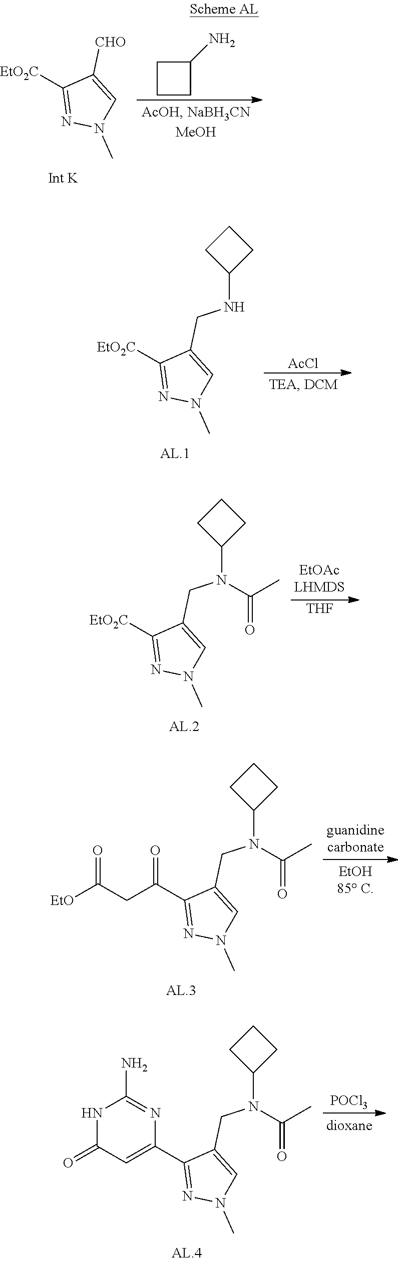

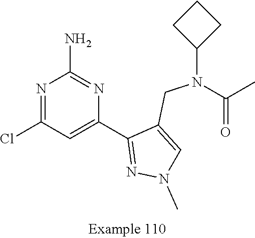 [SMILES: CC(=O)N(Cc1cn(C)nc1-c1cc(Cl)nc(N)n1)C1CCC1]

### Step 1

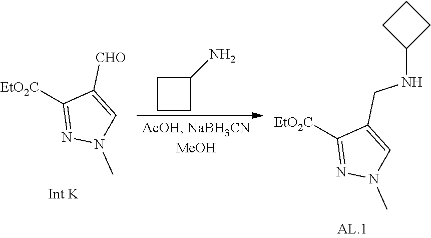

To a solution of ethyl 4-formyl-1-methyl-pyrazole-3-carboxylate (700 mg, 3.84 mmol, 1 eq) in MeOH (2 mL) under N₂ was added cyclobutanamine (273 mg, 3.84 mmol, 0.330 mL, 1 eq), and then AcOH (12 mg, 0.19 mmol, 0.011 mL, 0.05 eq) was added and the reaction mixture was stirred at 25° C. for 1 h. NaBH₃CN (483 mg, 7.68 mmol, 2 eq) was added and the reaction mixture was then stirred for 12 h at 25° C. The reaction was quenched with aqueous HCl (1M) to a final pH of 6-7. The mixture was extracted with EtOAc (50 mL×7). The organic was dried over Na₂SO₄, filtered and concentrated which furnished methyl 4-[(cyclobutylamino)methyl]-1-methyl-pyrazole-3-carboxylate. The crude product was used directly in next step without further purification.

### Step 2

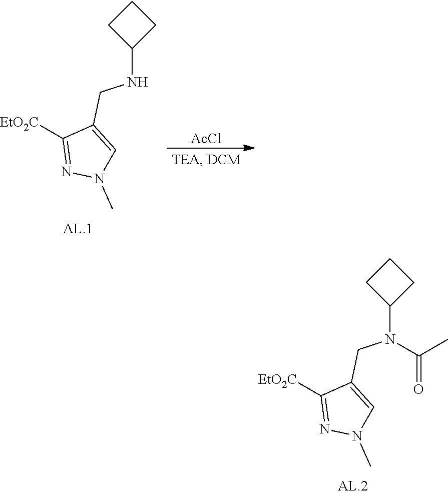 [SMILES: "AcCl" ; CCOC(=O)c1nn(C)cc1CNC1CCC1 ; CCOC(=O)c1nn(C)cc1CN(C(C)=O)C1CCC1]

To a stirred solution of ethyl 4-[(cyclobutylamino)methyl]-1-methyl-pyrazole-3-carboxylate (0.900 g, 3.79 mmol, 1 eq) and TEA (576 mg, 5.69 mmol, 0.792 mL, 1.5 eq) in DCM (10 mL) was added acetyl chloride (327 mg, 4.17 mmol, 0.298 mL, 1.1 eq) at 0° C. dropwise under N₂. After the addition, the mixture was allowed to warm to 25° C. and stirred at that temperature for 2 h. The reaction mixture was quenched by addition of MeOH (5 mL) and concentrated. The residue was purified by flash chromatography (ISCO®; 12 g SepaFlash@ Silica Flash Column, gradient elution of 0 to 100% ethyl acetate/petroleum ether @ 75 mL/min) which furnished ethyl 4-[[acetyl(cyclobutyl)amino]methyl]-1-methyl-pyrazole-3-carboxylate.

Example 110 was prepared from intermediate AL.2 using conditions similar to those outlined in steps 3-5 in Scheme C.

Example 110: ¹H NMR: (400 MHz, CD3OD) δ 7.42 (d, J=11.9 Hz, 1H), 7.18 (d, J=5.4 Hz, 1H), 4.99 (d, J=18.4 Hz, 2H), 4.52-4.35 (m, 1H), 3.90 (d, J=13.8 Hz, 3H), 2.25-2.11 (m, 5H), 2.11-2.02 (m, 2H), 1.72-1.55 (m, 2H); LCMS: (MH+) 335.1.

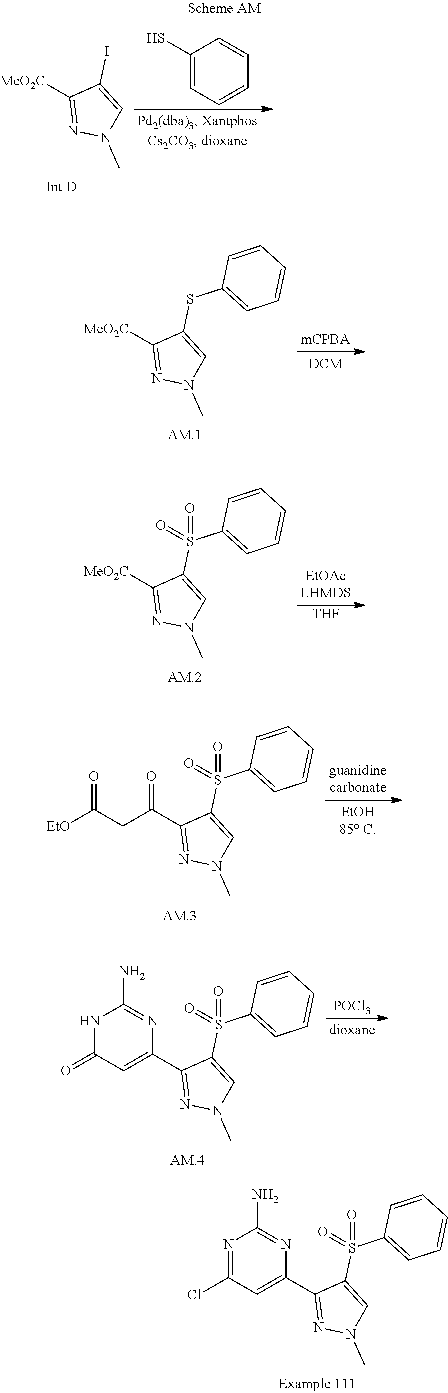

### Step 1

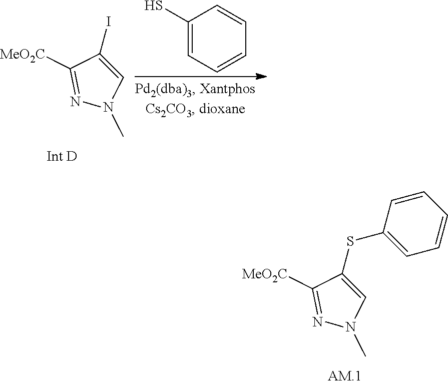 [SMILES: COC(=O)c1nn(C)cc1I ; Sc1ccccc1 ; COC(=O)c1nn(C)cc1Sc1ccccc1]

To a mixture of methyl 4-iodo-1-methyl-pyrazole-3-carboxylate (300 mg, 1.13 mmol, 1 eq) and Cs₂CO₃ (551 mg, 1.69 mmol, 1.5 eq) was added dioxane (20 mL). Benzenethiol (186 mg, 1.69 mmol, 0.173 mL, 1.5 eq) was added to the mixture, and the mixture was degassed/purged with N₂ three times. Pd₂(dba)₃ (103 mg, 0.113 mmol, 0.1 eq) and Xantphos (131 mg, 0.225 mmol, 0.2 eq) was added to the mixture, and the mixture was degassed/purged with N₂ three times. The mixture was stirred at 100° C. for 12 h under N₂. The reaction mixture was diluted with water (30 mL). The reaction mixture was filtered through Celite, and the filter cake was washed with EtOAc (100 mL×3). The organic layer was washed with brine (100 mL), dried over Na₂SO₄, and filtered. The filtrate was concentrated under reduced pressure. The residue was purified by flash silica gel chromatography (ISCO®; 4 g SepaFlash@ Silica Flash Column, gradient elution of 0 to 30% ethyl acetate/petroleum ether @ 36 mL/min) which furnished methyl-1-methyl-4-phenylsulfanyl-pyrazole-3-carboxylate.

### Step 2

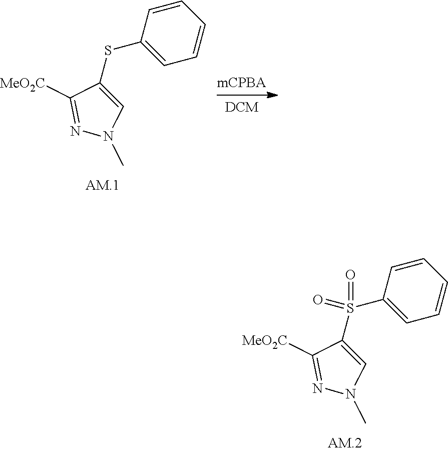 [SMILES: COC(=O)c1nn(C)cc1Sc1ccccc1 ; COC(=O)c1nn(C)cc1S(=O)(=O)c1ccccc1]

To a mixture of methyl 1-methyl-4-phenylsulfanyl-pyrazole-3-carboxylate (200 mg, 0.805 mmol, 1 eq) in DCM (2 mL) was added m-CPBA (521 mg, 2.42 mmol, 80% purity, 3 eq) at 0° C. The mixture was stirred at 25° C. for 12 h. The reaction mixture was diluted with sat. aqueous Na₂SO₃ (30 mL). The mixture was extracted with EtOAc (30 mL×3). The organic layer was washed with brine (50 mL), dried over Na₂SO₄, and filtered. The filtrate was concentrated under reduced pressure. The residue was purified by flash chromatography (ISCO®; 4 g SepaFlash@ Silica Flash Column, gradient elution of 0 to 30% ethyl acetate/petroleum ether @ 36 mL/min) which furnished methyl 4-(benzenesulfonyl)-1-methyl-pyrazole-3-carboxylate.

Example 111 was prepared from intermediate AM.2 using conditions similar to those depicted in steps 3-5 in Scheme C.

Example 111: ¹H NMR: (400 MHz, DMSO-d6) δ 8.66 (s, 1H), 7.99 (d, J=7.1 Hz, 2H), 7.66-7.54 (m, 3H), 6.89 (s, 1H), 3.96 (s, 3H); LCMS: (MH+) 350.1.

The following examples in Table 12 were prepared in a similar fashion to that depicted in Scheme V using the appropriate conditions in Step 10.

TABLE 12

| Ex. | Structure | Conditions | 1H NMR (400 MHz) | LCMS |
|---|---|---|---|---|
| 112 | 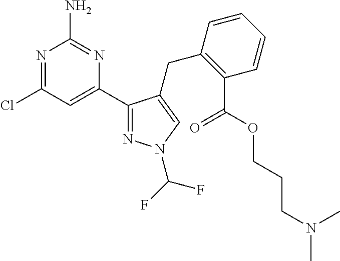 [SMILES: CN(C)CCCOC(=O)c1ccccc1Cc1cn(C(F)F)nc1-c1cc(Cl)nc(N)n1] | 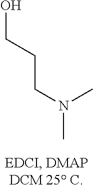 | (CD3OD) δ 7.88-7.83 (m, 1H), 7.61-7.41 (m, 3H), 7.37-7.30 (m, 2H), 7.22 (d, J = 1.0 Hz, 1H), 4.63 (s, 2H), 4.25 (t, J = 6.4 Hz, 2H), 2.39-2.30 (m, 2H), 2.19 (s, 6H), 1.82 (td, J = 7.0, 14.2 Hz, 2H | 465.2 |

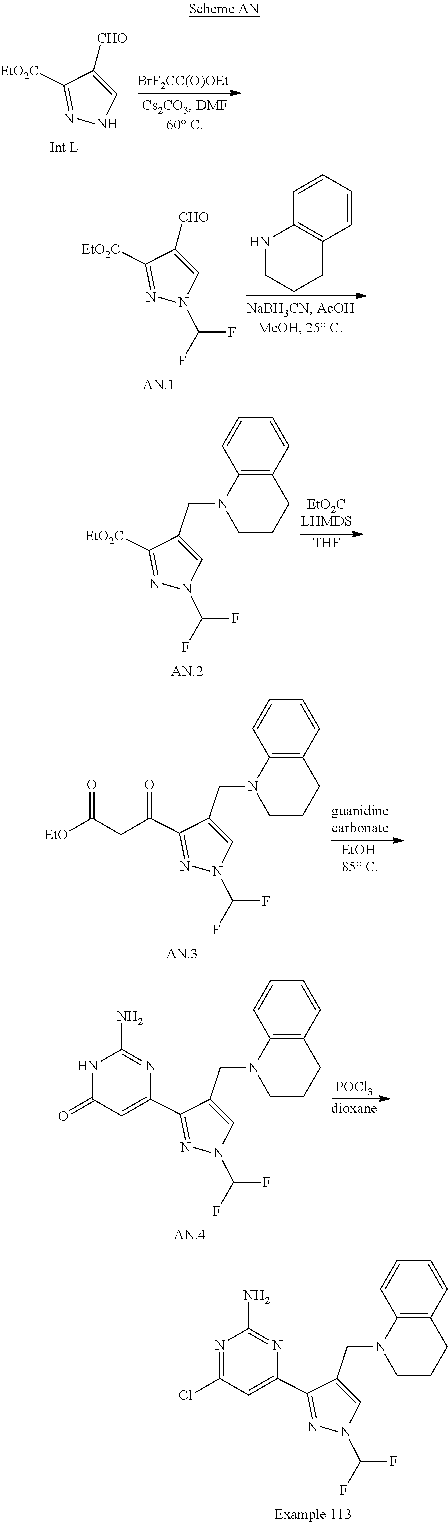

### Step 1

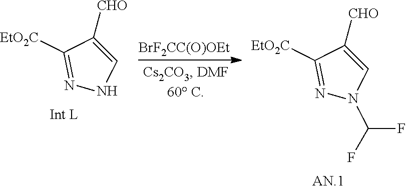 [SMILES: CCOC(=O)c1n[nH]cc1C=O ; CCOC(=O)c1nn(C(F)F)cc1C=O ; "BrF2CC(O)OEt"]

To a solution of ethyl 4-formyl-1H-pyrazole-3-carboxylate (1.70 g, 10.1 mmol, 1 eq) in DMF (15 mL) was added ethyl 2-bromo-2,2-difluoro-acetate (2.46 g, 12.1 mmol, 1.56 mL, 1.2 eq) and Cs₂CO₃ (6.59 g, 20.2 mmol, 2 eq). The reaction mixture was stirred at 60° C. for 12 h under N₂. The reaction mixture was quenched by addition of water (100 mL), and the mixture was extracted with EtOAc (50 mL×4). The combined organic layers were washed with brine (50 mL), dried over Na₂SO₄, and filtered. The filtrate was concentrated under reduced pressure. The residue was purified by flash chromatography (ISCO®; 40 g SepaFlash@ Silica Flash Column, gradient elution of 0 to 10% ethylacetate/petroleum ether @ 100 mL/min). The material was further purified by preparative-TLC (SiO₂, petroleum ether/EtOAc=5/1) which furnished ethyl 1-(difluoromethyl)-4-formyl-pyrazole-3-carboxylate.

### Step 2

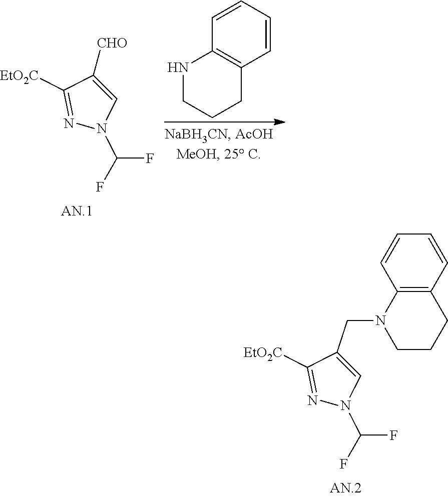 [SMILES: CCOC(=O)c1nn(C(F)F)cc1C=O ; c1ccc2c(c1)CCCN2 ; CCOC(=O)c1nn(C(F)F)cc1CN1CCCc2ccccc21]

Ethyl 1-(difluoromethyl)-4-formyl-pyrazole-3-carboxylate (600 mg, 2.75 mmol, 1 eq) and 1,2,3,4-tetrahydroquinoline (366 mg, 2.75 mmol, 1 eq) was dissolved in MeOH (18 mL) under N₂. AcOH (165 mg, 2.75 mmol, 0.157 mL, 1 eq) was added, and the reaction was stirred at 25° C. for 40 min. NaBH₃CN (346 mg, 5.50 mmol, 2 eq) was added, and the reaction was stirred for 12 h at 25° C. The reaction was quenched with H₂O (100 mL) and extracted with EtOAc (70 mL×4). The combined organic layers were washed with brine (50 mL), dried over Na₂SO₄, and filtered. The filtrate was concentrated under reduced pressure. The residue was purified by flash chromatography (ISCO®; 20 g SepaFlash@ Silica Flash Column, gradient elution of 0 to 10% ethylacetate/petroleum ether @ 75 mL/min) which furnished ethyl 1-(difluoromethyl)-4-(3,4-dihydro-2H-quinolin-1-ylmethyl) pyrazole-3-carboxylate.

Example 113 was prepared from intermediate AN.2 using conditions similar to those depicted in steps 3-5 of Scheme C.

Example 113: ¹H NMR: (400 MHz, CD3OD) δ 7.71 (s, 1H), 7.62-7.29 (m, 1H), 7.26 (s, 1H), 6.95-6.86 (m, 2H), 6.57-6.42 (m, 2H), 4.83 (s, 2H), 3.50-3.36 (m, 2H), 2.78 (t, J=6.3 Hz, 2H), 1.99 (quin, J=6.0 Hz, 2H); LCMS: (MH+) 391.1.

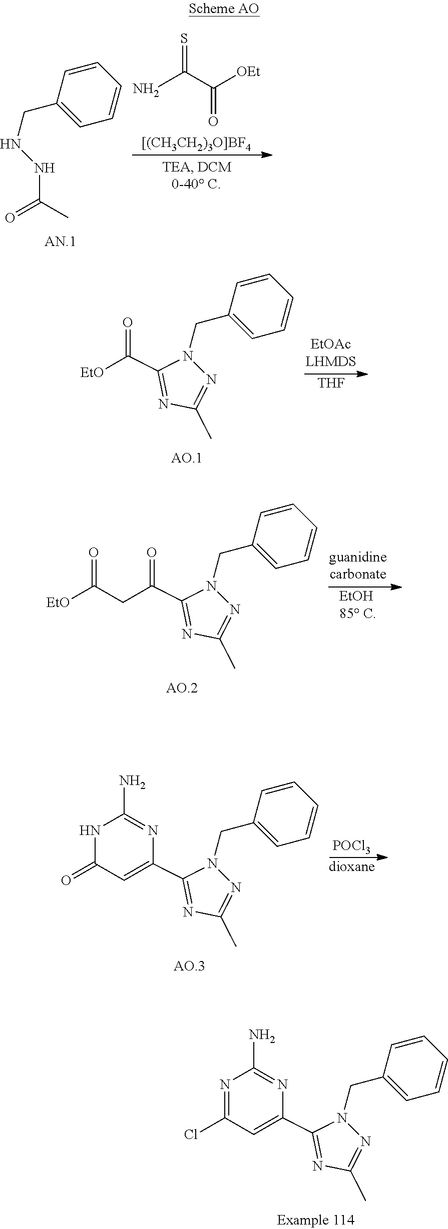

### Step 1

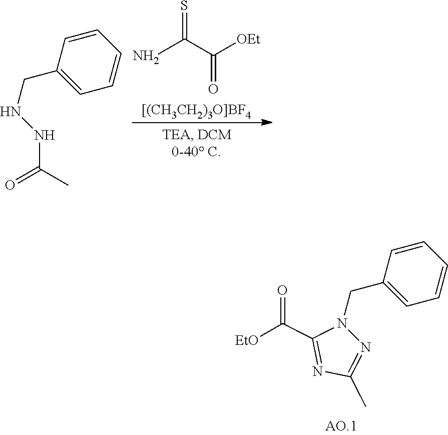

A mixture of ethyl 2-amino-2-thioxo-acetate (1.62 g, 12.2 mmol, 1 eq) and triethyloxoniumtetrafluoroborate (2.43 g, 12.8 mmol, 1.83 mL, 1.05 eq) in DCM (20 mL) was stirred at 20° C. for 2 h under N₂. After cooling the reaction to 0° C., a mixture of N′-benzylacetohydrazide (2.00 g, 12.2 mmol, 1 eq) and TEA (1.23 g, 12.2 mmol, 1.70 mL, 1 eq) in DCM (10 mL) was added slowly at 0° C. under N₂. After the addition, the mixture was stirred at 40° C. for 5 h under N₂. The mixture was concentrated under reduced pressure to remove the solvent. The residue was purified by column chromatography (SiO₂, petroleum ether/ethyl acetate=gradient of 4/1 to 7/3) which furnished 2-benzyl-5-methyl-1,2,4-triazole-3-carboxylate.

Example 114 was prepared from intermediate AO.1 using conditions similar to those depicted in steps 3-5 of Scheme C.

Example 114: ¹H NMR: (400 MHz, CD3OD) δ 7.32-7.23 (m, 6H), 6.02 (s, 2H), 2.38 (s, 3H); LCMS: (MH+) 301.1.

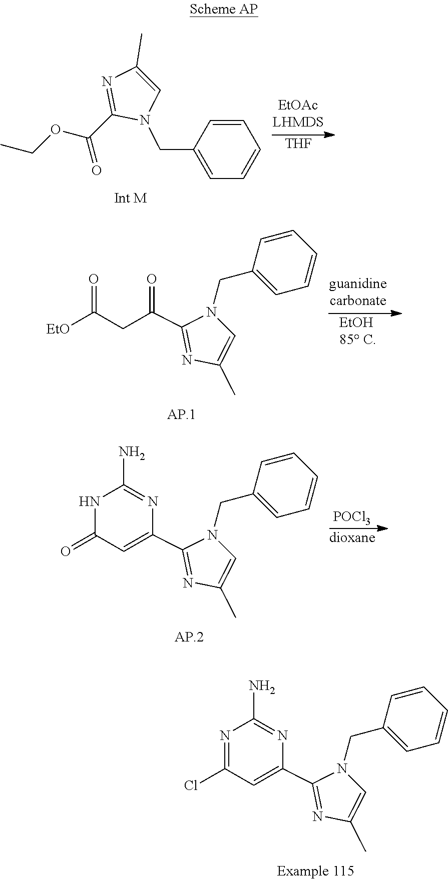

Example 115 was prepared from Int M using conditions similar to those depicted in steps 3-5 of Scheme C.

Example 115: ¹H NMR: (400 MHz, DMSO-d₆) δ 7.32-7.26 (m, 2H), 7.25-7.18 (m, 6H), 7.11 (s, 1H), 5.89 (s, 2H), 2.14 (s, 3H); LCMS: (MH+) 300.0.

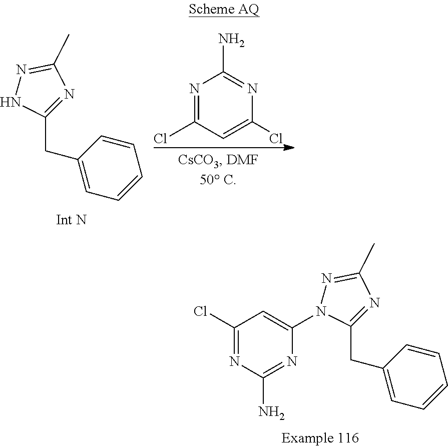

To a stirred mixture of 4,6-dichloropyrimidin-2-amine (80 mg, 0.48 mmol, 1.2 eq) in DMF (1 mL) was added 5-benzyl-3-methyl-1H-1,2,4-triazole (70 mg, 0.40 mmol, 1 eq) and Cs₂CO₃ (198 mg, 0.606 mmol, 1.5 eq). The mixture was stirred at 15° C. for 16 h and then at 50° C. for 13 h. The reaction mixture was diluted with water (50 mL). The solution was extracted with EtOAc (40 mL×3). The combined organic layer was washed with brine (60 mL), dried over Na₂SO₄, and filtered. The filtrate was concentrated under the reduced pressure to remove the solvent. The residue was purified by column chromatography (SiO₂, petroleum ether/ethyl acetate=4/1). 90 mg residue was obtained as yellow oil. The residue was further purified by neutral preparative-HPLC (column: Waters Xbridge BEH C18 100×25 mm, 5 m; mobile phase: [water (10 mM NH₄HCO₃)-ACN]; B %: 30%-60%, 8 min) which provided two peaks. The faster eluting peak thus obtained was Example 116.

Example 116: ¹H NMR: (DMSO-d6, 400 MHz) δ 7.55 (br s, 2H), 7.32-7.24 (m, 4H), 7.22-7.16 (m, 1H), 6.91 (s, 1H), 4.71 (s, 2H), 2.29 (s, 3H); LCMS: (MH+) 301.0.

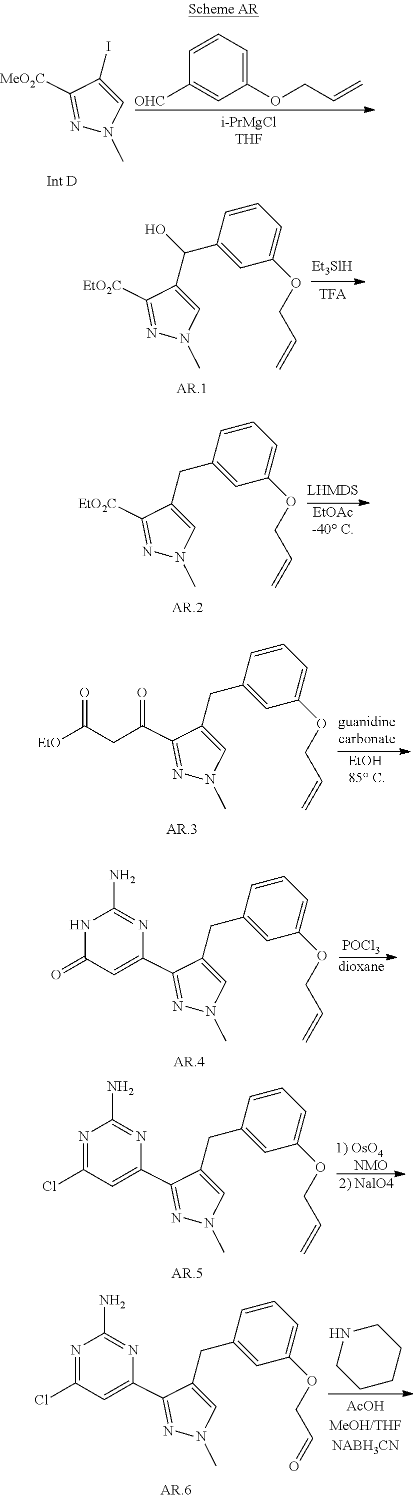

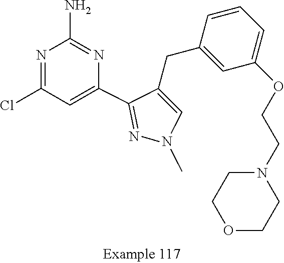 [SMILES: Cn1cc(Cc2cccc(OCCN3CCOCC3)c2)c(-c2cc(Cl)nc(N)n2)n1]

Example 117 was prepared from Int D using conditions similar to those depicted in Scheme AB.

Example 117: ¹H NMR: (400 MHz, CD3OD) δ 7.34 (s, 1H), 7.14 (br t, J=7.8 Hz, 1H), 7.09 (s, 1H), 6.88-6.79 (m, 2H), 6.72 (br d, J=7.7 Hz, 1H), 4.25 (s, 2H), 4.07 (br t, J=5.4 Hz, 2H), 3.88 (s, 3H), 3.73-3.68 (m, 4H), 2.76 (br t, J=5.4 Hz, 2H), 2.57 (br s, 4H); LCMS: (MH+) 429.0.

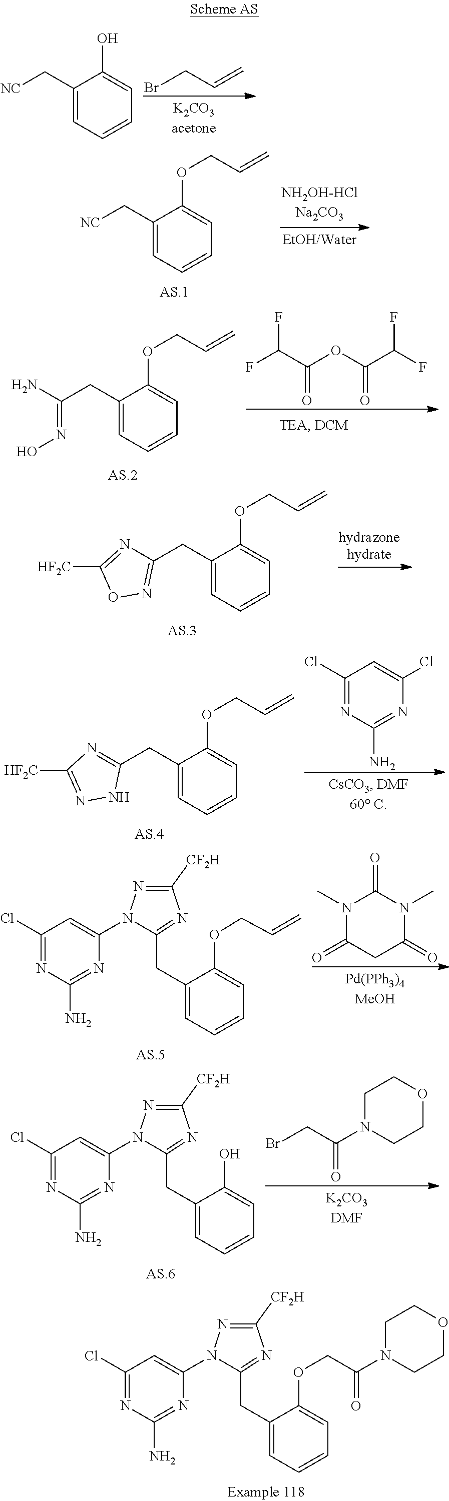

### Step 1

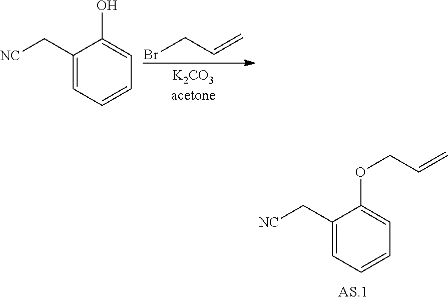

To a solution of 2-(2-hydroxyphenyl)acetonitrile (1.60 g, 12.0 mmol, 1 eq) in acetone (20 mL) was added K₂CO₃ (4.98 g, 36.1 mmol, 3 eq) and 3-bromoprop-1-ene (2.91 g, 24.0 mmol, 2 eq). The mixture was stirred at 25° C. for 12 h under N₂. The reaction mixture was filtered. The filter cake was washed with acetone (10 mL×3). The filtrate was concentrated. The residue was purified by flash chromatography (ISCO®; 20 g SepaFlash@ Silica Flash Column, gradient elution of 0 to 10% ethyl acetate/petroleum ether @ 75 mL/min) which furnished 2-(2-allyloxyphenyl)acetonitrile.

### Step 2

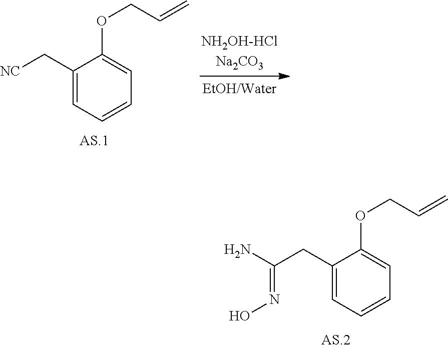

To a solution of 2-(2-allyloxyphenyl)acetonitrile (1.90 g, 11.0 mmol, 1 eq) in EtOH (18 mL) and H₂O (6 mL) was added Na₂CO₃ (2.33 g, 21.9 mmol, 2 eq) and hydroxylamine hydrochloride (1.52 g, 21.9 mmol, 2 eq). The mixture was stirred at 80° C. for 12 h under N₂. The reaction mixture was poured into H₂O (150 mL). The mixture was extracted with ethyl acetate (50 mL×3). The organic phase was washed with brine (80 mL), dried over anhydrous Na₂SO₄, and filtered. The filtrate was concentrated under reduced pressure. The residue was purified by flash chromatography (ISCO®; 40 g SepaFlash® Silica Flash Column, gradient elution of 0 to 40% ethyl acetate/petroleum ether @ 100 mL/min) which furnished 2-(2-allyloxyphenyl)-N′-hydroxy-acetamidine.

### Step 3

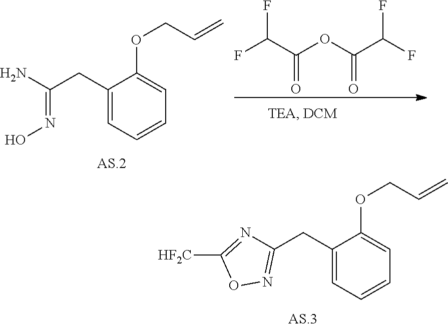

To a solution of 2-(2-allyloxyphenyl)-N′-hydroxy-acetamidine (1.80 g, 8.73 mmol, 1 eq) in DCM (20 mL) was added TEA (1.06 g, 10.5 mmol, 1.46 mL, 1.2 eq) and (2,2-difluoroacetyl) 2,2-difluoroacetate (3.04 g, 17.5 mmol, 2 eq). The mixture was stirred at 45° C. for 3 h under N₂. The reaction mixture was concentrated under reduced pressure. The residue was purified by flash chromatography (ISCO®; 40 g SepaFlash® Silica Flash Column, gradient elution of 0 to 6% ethyl acetate/petroleum ether @ 75 mL/min) which furnished 3-[(2-allyloxy phenyl)methyl]-5-(difluoromethyl)-1,2,4-oxadiazole.

### Step 4

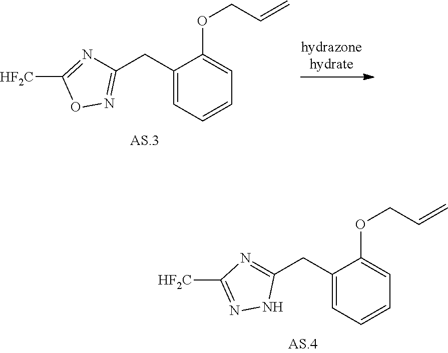

To a solution of 3-[(2-allyloxyphenyl)methyl]-5-(difluoromethyl)-1,2,4-oxadiazole (2.00 g, 7.51 mmol, 1 eq) in DMF (20 mL) was added NH₂NH₂·H₂O (3.84 g, 75.1 mmol, 3.73 mL, 98% purity, 10 eq). The mixture was stirred at 25° C. for 36 h under N₂. The reaction mixture was poured into H₂O (150 mL). The mixture was extracted with ethyl acetate (50 mL×5). The organic phase was washed with brine (80 mL), dried over anhydrous Na₂SO₄, and filtered. The filtrate was concentrated under reduced pressure. The residue was purified by flash chromatography (ISCO®; 20 g SepaFlash@ Silica Flash Column, gradient elution of 0 to 25% ethyl acetate/petroleum ether @ 75 mL/min) which furnished 5-[(2-allyloxyphenyl)methyl]-3-(difluoromethyl)-1H-1,2,4-triazole.

### Step 5

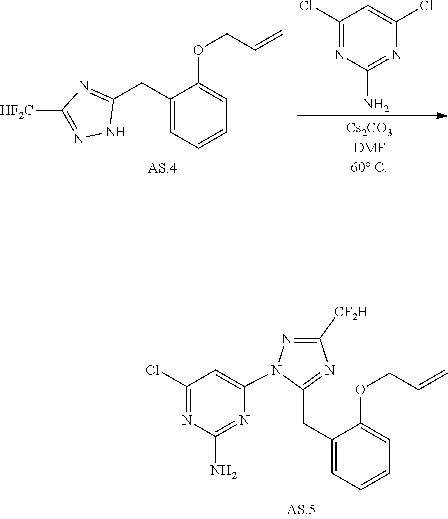

To a solution of 5-[(2-allyloxyphenyl)methyl]-3-(difluoromethyl)-1H-1,2,4-triazole (1.85 g, 6.97 mmol, 1 eq) in DMF (20 mL) was added Cs₂CO₃ (3.41 g, 10.5 mmol, 1.5 eq) and 4,6-dichloropyrimidin-2-amine (1.37 g, 8.37 mmol, 1.2 eq). The mixture was stirred at 25° C. for 12 h and then at 60° C. for another 12 h under N₂. The reaction mixture was poured into H₂O (150 mL). The mixture was extracted with ethyl acetate (50 mL×3). The organic phase was washed with brine (80 mL), dried over anhydrous Na₂SO₄, and filtered. The filtrate was concentrated in under reduced pressure. The residue was purified by flash chromatography (ISCO®; 12 g SepaFlash@ Silica Flash Column, gradient elution of 0 to 10% ethyl acetate/petroleum ether @ 75 mL/min) which provided 4-[5-[(2-allyloxyphenyl)methyl]-3-(difluoromethyl)-1,2,4-triazol-1-yl]-6-chloro-pyrimidin-2-amine.

### Step 6

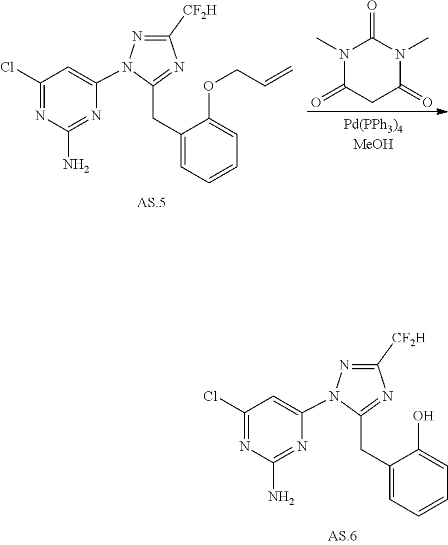

To a solution of 4-[5-[(2-allyloxyphenyl)methyl]-3-(difluoromethyl-1,2,4-triazol-1-yl]-6-chloro-pyrimidin-2-amine (440 mg, 1.12 mmol, 1 eq) in MeOH (5 mL) was added Pd(PPh₃)₄ (129 mg, 0.112 mmol, 0.1 eq) and 1,3-dimethylhexahydropyrimidine-2,4,6-trione (350 mg, 2.24 mmol, 2 eq) successively. The mixture was stirred at 25° C. for 2 h under N₂. The reaction mixture was poured into saturated, aqeuous sodium bicarbonate solution (100 mL). The mixture was extracted with ethyl acetate (30 mL×3). The organic phase was washed with brine (50 mL), dried over anhydrous Na₂SO₄, and filtered. The filtrate was concentrated under reduced pressure. The crude product residue was purified by flash chromatography (ISCO®; 12 g SepaFlash@ Silica Flash Column, gradient elution of 0 to 30% ethyl acetate/petroleum ether @ 60 mL/min) which furnished 2-[[2-(2-amino-6-chloro-pyrimidin-4-yl)-5-(difluoromethyl)-1,2,4-triazol-3-yl]methyl]phenol.

### Step 7

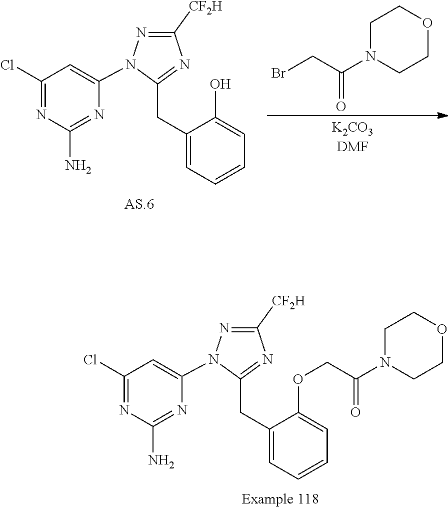 [SMILES: Nc1nc(Cl)cc(-n2nc(C(F)F)nc2Cc2ccccc2O)n1 ; O=C(CBr)N1CCOCC1 ; Nc1nc(Cl)cc(-n2nc(C(F)F)nc2Cc2ccccc2OCC(=O)N2CCOCC2)n1]

To a solution of 2-[[2-(2-amino-6-chloro-pyrimidin-4-yl)-5-(difluoromethyl)-1,2,4-triazol-3-yl]methyl]phenol (210 mg, 0.595 mmol, 1 eq) in DMF (3 mL) was added K₂CO₃ (165 mg, 1.19 mmol, 2 eq) and 2-bromo-1-morpholino-ethanone (124 mg, 0.595 mmol, 1 eq). The mixture was stirred at 25° C. for 1 h under N₂. The reaction mixture was poured into H₂O (50 mL). The mixture was extracted with ethyl acetate (20 mL×3). The organic phase was washed with brine (30 mL), dried over anhydrous Na₂SO₄, and filtered. The filtrate was concentrated under reduced pressure. The residue was purified by preparative-HPLC (column: Waters Xbridge BEH C18 100×30 mm, 10 m; mobile phase: [water (10 mM NH₄HCO₃)-ACN]; B %: 20%-50%, 10 min) to give two compounds. The compound was further purified by preparative-HPLC (column: Phenomenex Luna C18 100×30 mm, 5 m; mobile phase: [water (0.2% FA)-ACN]; B %: 35%-45%, 14 min) which furnished 2-[2-[[2-(2-amino-6-chloro-pyrimidin-4-yl)-5-(difluoromethyl)-1,2,4-triazol-3-yl]methyl]phenoxy]-1-morpholino-ethanone Example 118.

Example 118: ¹H NMR: (400 MHz, DMSO-d6) δ 7.61 (br s, 2H), 7.26-7.10 (m, 3H), 7.01-6.98 (m, 1H), 6.95-6.87 (m, 2H), 4.75 (s, 2H), 4.71 (s, 2H), 3.51 (br s, 4H), 3.38 (br s, 4H); LCMS: (MH+) 480.1.

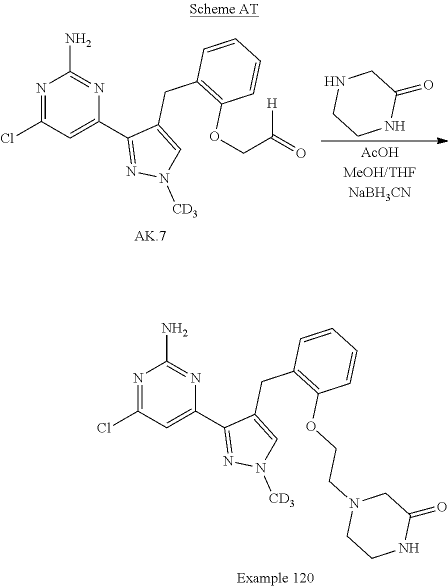 [SMILES: [2H]C([2H])([2H])n1cc(Cc2ccccc2OCC=O)c(-c2cc(Cl)nc(N)n2)n1 ; O=C1CNCCN1 ; [2H]C([2H])([2H])n1cc(Cc2ccccc2OCCN2CCNC(=O)C2)c(-c2cc(Cl)nc(N)n2)n1]

Example 120 was prepared from intermediate AK.7 following similar conditions depicted in Scheme AK using the appropriate reagents.

Example 120: ¹H NMR: (400 MHz, DMSO-d6) δ 7.73 (br s, 1H), 7.37 (s, 1H), 7.23-7.19 (m, 1H), 7.18-7.12 (m, 1H), 7.07 (s, 2H), 6.98 (s, 1H), 6.95 (d, J=7.9 Hz, 1H), 6.82 (t, J=7.3 Hz, 1H), 4.22 (s, 2H), 4.07 (t, J=5.3 Hz, 2H), 3.11-3.07 (m, 2H), 3.03 (s, 2H), 2.73 (t, J=5.4 Hz, 2H), 2.62-2.59 (m, 2H); LCMS: (MH+) 445.2.

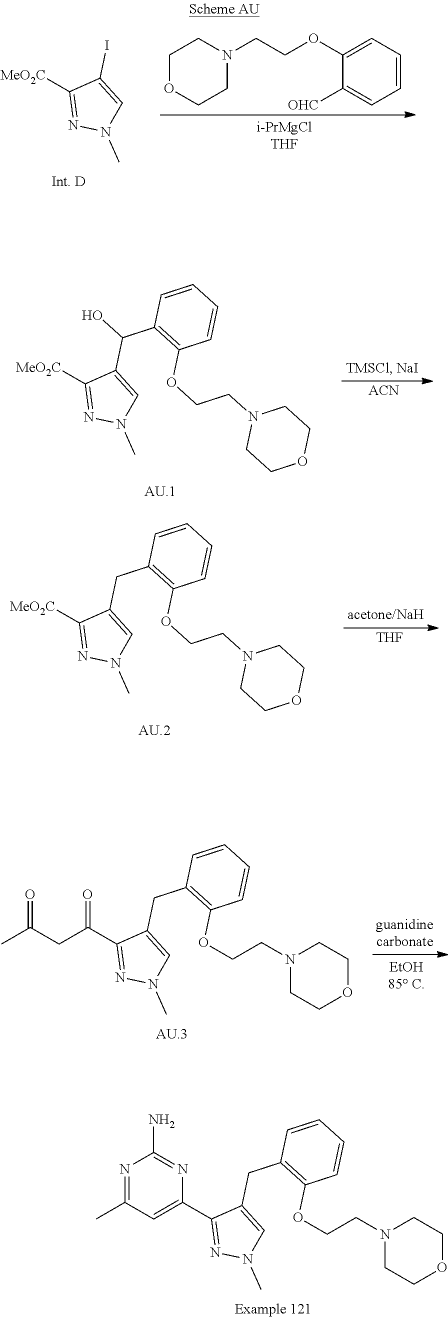

Intermediate AU.2 was prepared from Int. D using conditions similar to those outlined in Steps 1-2 of Scheme B.

### Step 3

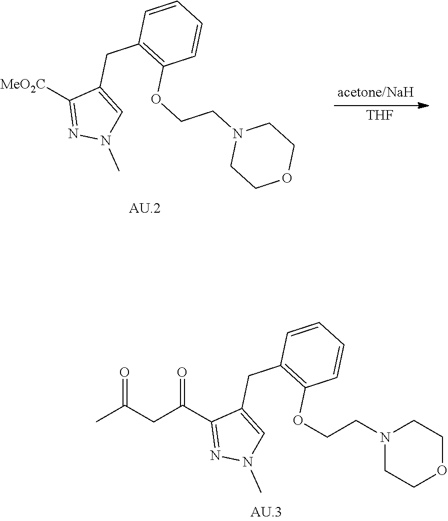 [SMILES: COC(=O)c1nn(C)cc1Cc1ccccc1OCCN1CCOCC1 ; "THF" ; CC(=O)CC(=O)c1nn(C)cc1Cc1ccccc1OCCN1CCOCC1]

To a solution of methyl 1-methyl-4-[[2-(2-morpholinoethoxy)phenyl]methyl] pyrazole-3-carboxylate (200 mg, 0.556 mmol, 1 eq) in THF (3 mL) was added NaH (67 mg, 1.67 mmol, 60 wt % dispersion in oil, 3 eq) at 20° C. The mixture was stirred at 20° C. for 0.5 h under N₂. Acetone (36 mg, 0.61 mmol, 0.045 mL, 1.1 eq) was added to the mixture at 20° C. The mixture was stirred at 20° C. for 1.5 h. The reaction was then stirred at 80° C. for 12 hr under N₂. The reaction mixture was quenched with H₂O (30 mL), and the mixture was extracted with EtOAc (20 mL×3). The organic layer was washed with brine (20 mL), dried over Na₂SO₄, and filtered. The filtrate was concentrated under reduced pressure which furnished 1-[1-methyl-4-[[2-(2-morpholinoethoxy) phenyl]methyl]pyrazol-3-yl]butane-1,3-dione (120 mg).

### Step 4

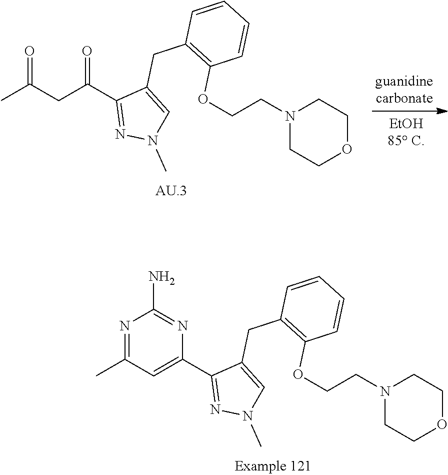 [SMILES: CC(=O)CC(=O)c1nn(C)cc1Cc1ccccc1OCCN1CCOCC1 ; Cc1cc(-c2nn(C)cc2Cc2ccccc2OCCN2CCOCC2)nc(N)n1]

To a solution of 1-[1-methyl-4-[[2-(2-morpholinoethoxy)phenyl]methyl]pyrazol-3-yl] butane-1,3-dione (120 mg, 0.311 mmol, 1 eq) in EtOH (5 mL) was added guanidine carbonate (112 mg, 0.622 mmol, 2 eq). The mixture was stirred at 85° C. for 12 hr under N₂. The reaction mixture was diluted with H₂O (30 mL), and the mixture was extracted with EtOAc (20 mL×3). The combined organic layers were washed with brine (20 mL), dried over Na₂SO₄, and filtered. The filtrate was concentrated under reduced pressure. The residue was purified by preparative-HPLC (column: Waters Xbridge Prep OBD C18 150×40 mm, 10 m; mobile phase: [water (10 mM NH₄HCO₃)-ACN]; B %: 25%-45%, 8 min) which furnished 4-methyl-6-[1-methyl-4-[[2-(2-morpholinoethoxy)phenyl]methyl]pyrazol-3-yl]pyrimidin-2-amine Example 121.

Example 121: ¹H NMR: (400 MHz, CDCl3) δ 7.22-7.14 (m, 2H), 7.06 (s, 1H), 7.00 (s, 1H), 6.91-6.84 (m, 2H), 4.95 (br s, 2H), 4.24 (s, 2H), 4.12 (t, J=5.6 Hz, 2H), 3.87 (s, 3H), 3.69-3.63 (m, 4H), 2.76 (t, J=5.6 Hz, 2H), 2.54-2.49 (m, 4H), 2.36 (s, 3H); LCMS: (MH+) 409.2.

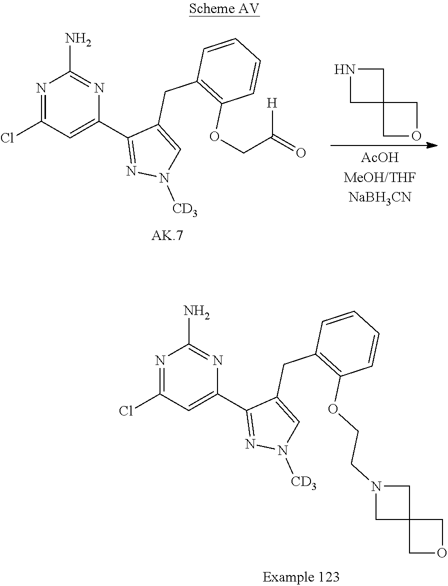 [SMILES: [2H]C([2H])([2H])n1cc(Cc2ccccc2OCC=O)c(-c2cc(Cl)nc(N)n2)n1 ; C1NCC12COC2 ; [2H]C([2H])([2H])n1cc(Cc2ccccc2OCCN2CC3(COC3)C2)c(-c2cc(Cl)nc(N)n2)n1]

Example 123 was prepared from intermediate K.7 using conditions similar to those depicted in Scheme AK.

Example 123: ¹H NMR: (400 MHz, CD3OD) δ 7.22-7.15 (m, 3H), 6.97 (s, 1H), 6.90 (d, J=7.6 Hz, 2H), 4.58 (s, 4H), 4.26 (s, 2H), 3.95 (t, J=4.9 Hz, 2H), 3.36 (s, 4H), 2.72 (t, J=4.9 Hz, 2H); LCMS: (MH+) 444.2.

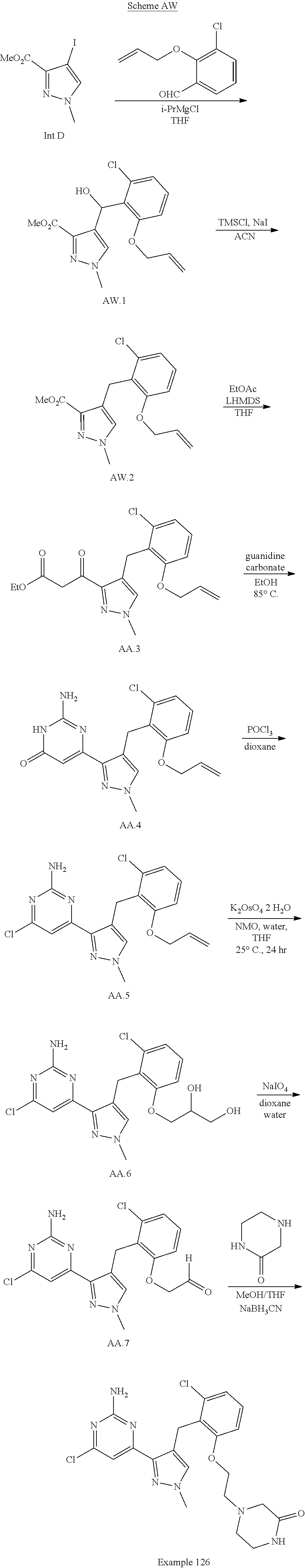

Example 126 was prepared from Int D using conditions similar to that depicted in Scheme AA.

Example 126: ¹H NMR: (DMSO-d6, 400 MHz) δ 7.68 (br s, 1H), 7.29-7.21 (m, 1H), 7.10 (s, 2H), 7.07-7.01 (m, 3H), 6.99 (s, 1H), 4.35 (s, 2H), 4.07 (t, J=5.4 Hz, 2H), 3.76 (s, 3H), 2.97-2.90 (m, 4H), 2.60 (t, J=5.4 Hz, 2H), 2.48-2.45 (m, 2H); LCMS: (MH+) 476.1.

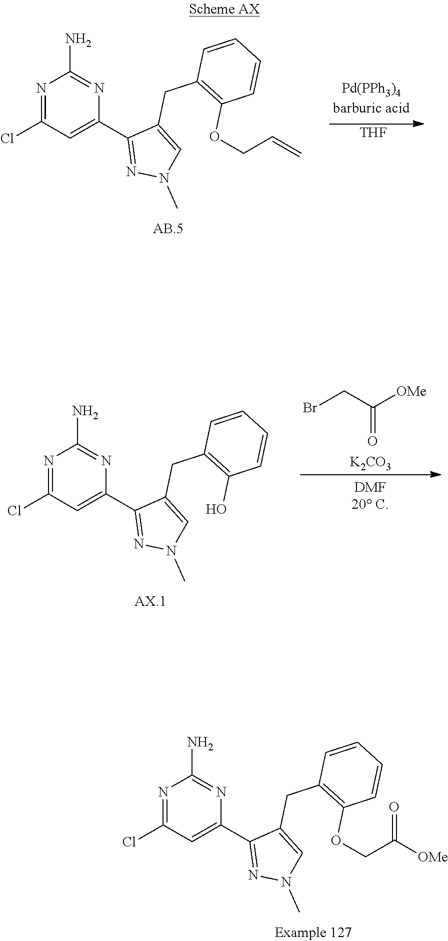

### Step 1

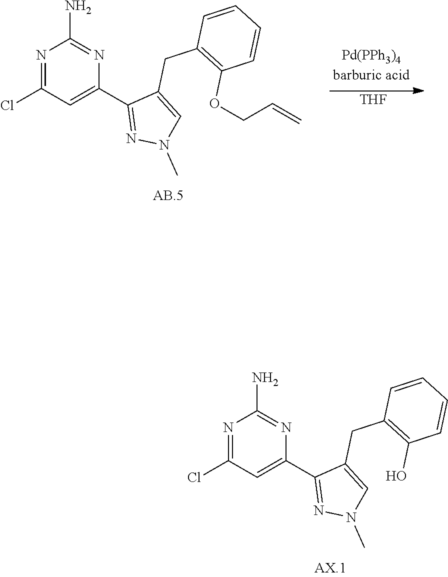 [SMILES: C=CCOc1ccccc1Cc1cn(C)nc1-c1cc(Cl)nc(N)n1 ; Cn1cc(Cc2ccccc2O)c(-c2cc(Cl)nc(N)n2)n1 ; "THF"]

To a solution of 4-[4-[(2-allyloxyphenyl)methyl]-1-methyl-pyrazol-3-yl]-6-chloro-pyrimidin-2-amine (0.100 g, 0.281 mmol, 1 eq) in THF (2 mL) was added Pd(PPh₃)₄ (32 mg, 0.028 mmol, 0.1 eq) and 1,3-dimethylhexahydropyrimidine-2,4,6-trione (88 mg, 0.56 mmol, 2 eq) successively. The mixture was stirred at 90° C. for 12 h under N₂. Pd(PPh₃)₄ (32 mg, 0.028 mmol, 0.1 eq) was added to the mixture. The mixture was stirred at 90° C. for another 18 h under N₂. The reaction mixture was diluted with water (30 mL). The solution was extracted with EtOAc (20 mL×3). The combined organic layers were washed with sat. aqueous NaHCO₃ (30 mL×2), brine (50 mL), dried over Na₂SO₄, and filtered. The filtrate was concentrated under the reduced pressure to remove the solvent. The residue was purified by column chromatography (SiO₂, petroleum ether/ethyl acetate=79:21) which furnished 2-[[3-(2-amino-6-chloro-pyrimidin-4-yl)-1-methyl-pyrazol-4-yl]methyl]phenol.

### Step 2

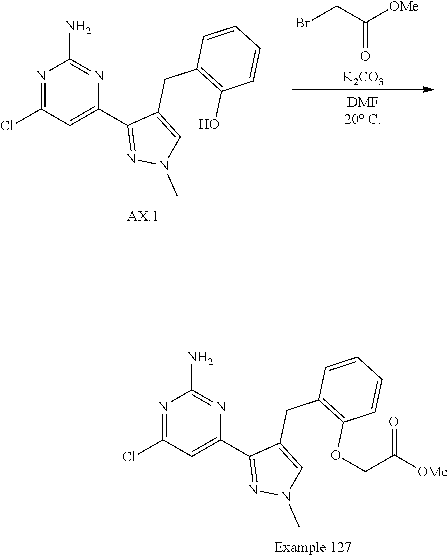

To a solution of 2-[[3-(2-amino-6-chloro-pyrimidin-4-yl)-1-methyl-pyrazol-4-yl]methyl]phenol (40 mg, 0.13 mmol, 1 eq) in DMF (1 mL) was added methyl 2-bromoacetate (23 mg, 0.15 mmol, 0.014 mL, 1.2 eq) and K₂CO₃ (53 mg, 0.380 mmol, 3 eq). The reaction mixture was stirred at 20° C. for 12 h under N₂. The reaction mixture was quenched with saturated aqueous NH₄Cl (50 mL). The mixture was extracted with 2-methyl tetrahydrofuran (20 mL×3). The combined organic layers were washed with brine (20 mL), dried over Na₂SO₄, and filtered. The filtrate was concentrated under reduced pressure. The residue was purified by preparative-HPLC (column: Waters Xbridge BEH C18 100×25 mm, 5 m; mobile phase: [water (10 mM NH₄HCO₃)-ACN]; B %: 30%-60%, 10 min) which furnished methyl 2-[2-[[3-(2-amino-6-chloro-pyrimidin-4-yl)-1-methyl-pyrazol-4-yl]methyl]phenoxy]acetate Example 127.

Example 127: ¹H NMR: (CDCl3, 400 MHz) δ 7.27-7.26 (m, 1H), 7.25 (s, 1H), 7.19-7.13 (m, 1H), 7.10 (d, J=7.5 Hz, 1H), 6.95-6.86 (m, 1H), 6.73 (d, J=8.1 Hz, 1H), 5.24 (br s, 2H), 4.70 (s, 2H), 4.30 (s, 2H), 3.91 (s, 3H), 3.82 (s, 3H); LCMS: (MH+) 388.1.

The following examples in Table 13 were prepared in a similar fashion to that depicted in Scheme AX using the appropriates conditions in Step 2.

TABLE 13

| Ex. | Structure | Conditions | 1H NMR (400 MHz) | LCMS |
|---|---|---|---|---|
| 128 | 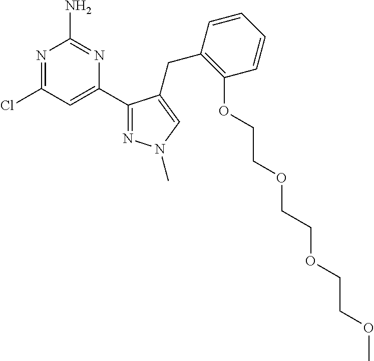 [SMILES: COCCOCCOCCOc1ccccc1Cc1cn(C)nc1-c1cc(Cl)nc(N)n1] | 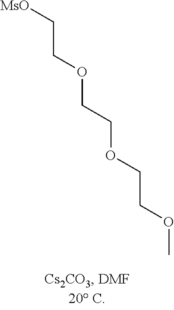 | (DMSO-d6) δ 7.42 (s, 1H), 7.22 (dd, J = 1.6, 7.5 Hz, 1H), 7.14 (dt, J = 1.7, 7.8 Hz, 1H), 7.05 (s, 2H), 6.99 (s, 1H), 6.94 (d, J = 7.6 Hz, 1H), 6.82 (dt, J = 0.9, 7.4 Hz, 1H), 4.24 (s, 2H), 4.11-4.06 (m, 2H), 3.81 (s, 3H), 3.74 (dd, J = 3.9, 5.3 Hz, 2H), 3.60-3.55 (m, 2H), 3.53-3.47 (m, 4H), 3.41-3.37 (m, 2H), 3.21 (s, 3H) | 462.2 |
| 134 | 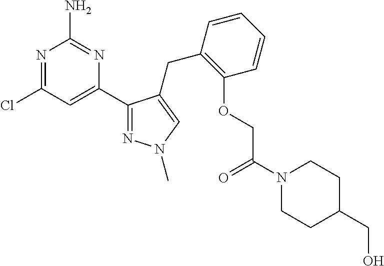 [SMILES: Cn1cc(Cc2ccccc2OCC(=O)N2CCC(CO)CC2)c(-c2cc(Cl)nc(N)n2)n1] | 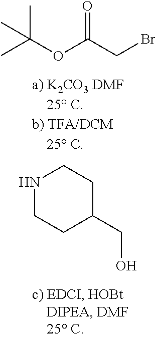 | (CDCl3) δ 7.25 (s, 1H), 7.22-7.15 (m, 1H), 7.13-7.08 (m, 2H), 6.90 (t, J = 7.1 Hz, 1H), 6.86 (d, J = 8.2 Hz, 1H), 5.29 (br s, 2H), 4.80-4.65 (m, 2H), 4.60 (br d, J = 13.7 Hz, 1H), 4.26 (d, J = 3.4 Hz, 2H), 3.89 (s, 4H), 3.57-3.33 (m, 2H), 2.99 (br t, J = 11.8 Hz, 1H), 2.70-2.56 (m, 1H), 1.72 (br d, J = 6.8 Hz, 3H), 1.54 (br s, 1H), 1.22-0.98 (m, 2H) | 471.2 |
| 142 | 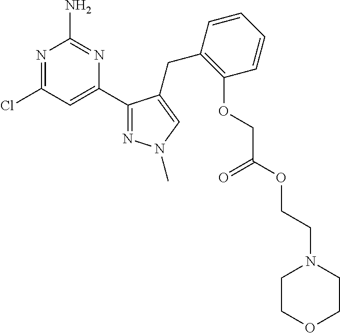 [SMILES: Cn1cc(Cc2ccccc2OCC(=O)OCCN2CCOCC2)c(-c2cc(Cl)nc(N)n2)n1] | 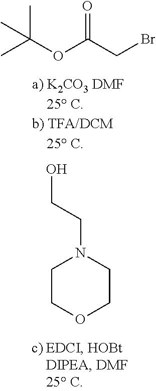 | (DMSO-d6, formic acid salt) δ 8.13 (s, 1H), 7.43 (s, 1H), 7.20 (br d, J = 7.3 Hz, 1H), 7.16-7.09 (m, 1H), 7.05 (br s, 2H), 6.99 (s, 1H), 6.88-6.82 (m, 2H), 4.82 (s, 2H), 4.31- 4.21 (m, 4H), 3.81 (s, 3H), 3.57-3.46 (m, 4H), 2.54 (br s, 2H), 2.37 (br s, 4H) | 487.2 |

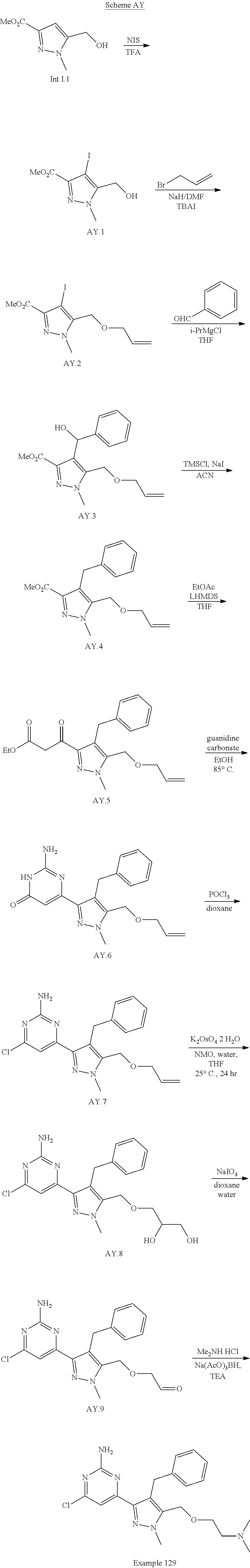

### Step 1

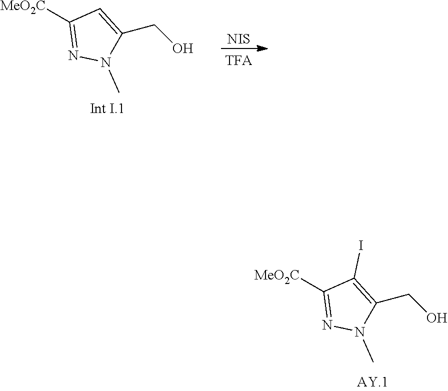

To a solution of methyl 5-(hydroxymethyl)-1-methyl-pyrazole-3-carboxylate (5.40 g, 31.7 mmol, 1 eq) in TFA (55 mL) was added NIS (7.14 g, 31.7 mmol, 1 eq). The mixture was stirred at 15° C. for 12 hr under N₂. The reaction mixture was concentrated to remove TFA. The mixture was poured into saturated, aqueous sodium bicarbonate solution (150 mL). The mixture was adjusted to pH=7 by addition of sat. aqueous Na₂CO₃. The mixture was extracted with ethyl acetate (70 mL×3). The mixture was adjusted to pH=5 by addition of TFA. The mixture was extracted with ethyl acetate (70 mL×3). The organic phase was washed with sat. aqueous sodium bicarbonate solution (50 mL), brine (80 mL), dried over anhydrous Na₂SO₄, and filtered. The filtrate was concentrated under reduced pressure which furnished methyl 5-(hydroxymethyl)-4-iodo-1-methyl-pyrazole-3-carboxylate. The material was used directly in the next step without further purification.

### Step 2

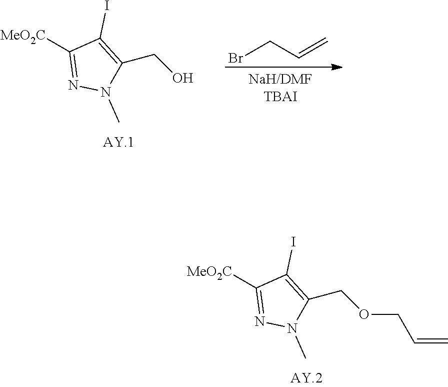

To a mixture of methyl 5-(hydroxymethyl)-4-iodo-1-methyl-pyrazole-3-carboxylate (12.0 g, 40.5 mmol, 1 eq) in DMF (120 mL) was added NaH (3.24 g, 81.1 mmol, 60 wt % dispersion in oil) at 0° C. After stirring the mixture at 0° C. for 0.5 h, 3-bromoprop-1-ene (14.7 g, 122 mmol, 3 eq) and TBAI (1.50 g, 4.05 mmol, 0.1 eq) was added to the mixture at 0° C. The mixture was stirred at 20° C. for 2 h under N₂. The reaction mixture was diluted with sat. aqueous NH₄Cl solution (200 mL). Then the mixture was extracted with EtOAc (100 mL×3). The organic layer was washed with brine (200 mL), dried over Na₂SO₄, and filtered. The filtrate was concentrated under reduced pressure. The residue was purified by flash chromatography (ISCO®; 120 g SepaFlash@ Silica Flash Column, gradient elution of 0 to 20% ethyl acetate/petroleum ether @ 100 mL/min) to give methyl 5-(allyloxymethyl)-4-iodo-1-methyl-pyrazole-3-carboxylate.

Example 129 was prepared in a similar fashion to that depicted in Scheme AB.

Example 129: ¹H NMR: (400 MHz, CD3OD) δ 7.21-7.13 (m, 4H), 7.12-7.05 (m, 2H), 4.52 (s, 2H), 4.40 (s, 2H), 3.95 (s, 3H), 3.44 (t, J=5.6 Hz, 2H), 2.47 (t, J=5.6 Hz, 2H), 2.20 (s, 6H); LCMS: (MH+) 401.2.

The examples in Table 14 were prepared in a similar fashion to that depicted in Scheme AI using the appropriate conditions in Step 9.

TABLE 14

| Ex. | Structure | Conditions | 1H NMR (400 MHz) | LCMS |
|---|---|---|---|---|
| 143 | 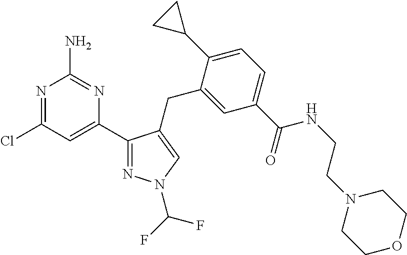 [SMILES: Nc1nc(Cl)cc(-c2nn(C(F)F)cc2Cc2cc(C(=O)NCCN3CCOCC3)ccc2C2CC2)n1] | 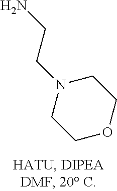 | (DMSO-d6) δ 8.26 (br t, J = 5.6 Hz, 1H), 7.92-7.62 (m, 4H), 7.27 (br s, 2H), 7.07 (s, 1H), 7.01 (d, J = 8.1 Hz, 1H), 4.47 (s, 2H), 3.55 (t, J = 4.5 Hz, 4H), 3.37-3.35 (m, 1.39H), 3.30 (br s, 0.73H), 2.45-2.41 (m, 2H), 2.40 (br d, J = 6.8 Hz, 4H), 2.00-1.90 (m, 1H), 0.89-0.82 (m, 2H), 0.64 (q, J = 5.1 Hz, 2H) | 532.2 |
| 144 | 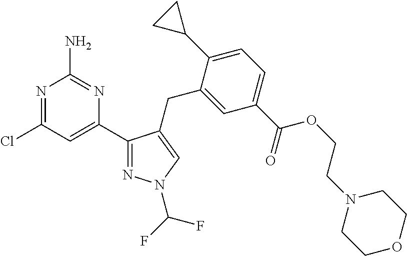 [SMILES: Nc1nc(Cl)cc(-c2nn(C(F)F)cc2Cc2cc(C(=O)OCCN3CCOCC3)ccc2C2CC2)n1] | 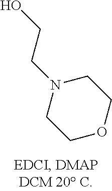 | (DMSO-d6) δ 7.93- 7.63 (m, 4H), 7.24 (br s, 2H), 7.12-7.02 (m, 2H), 4.52 (s, 2H), 4.32 (t, J = 5.7 Hz, 2H), 3.57-3.50 (m, 4H), 2.63 (t, J = 5.7 Hz, 2H), 2.45-2.40 (m, 4H), 2.09-1.99 (m, 1H), 0.96-0.89 (m, 2H), 0.72-0.66 (m, 2H) | 533.2 |

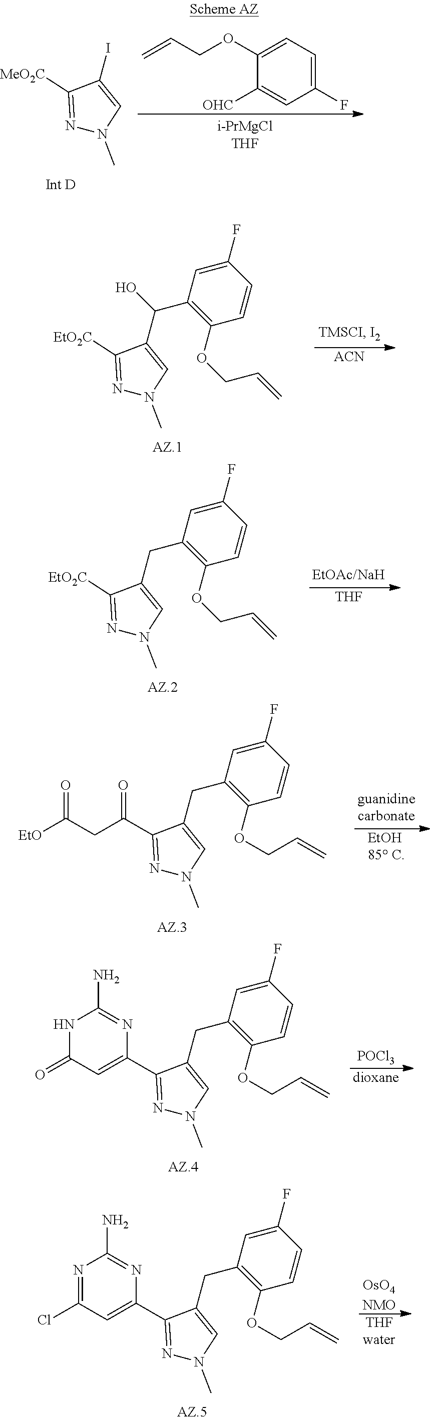

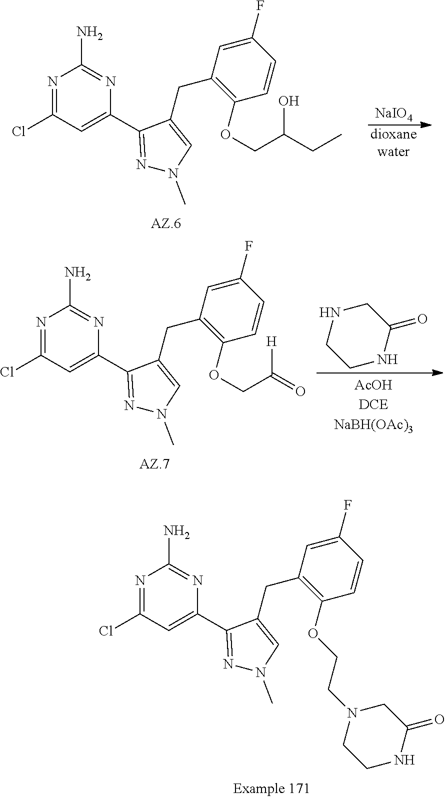 [SMILES: CCC(O)COc1ccc(F)cc1Cc1cn(C)nc1-c1cc(Cl)nc(N)n1 ; "NaIO4" ; "dioxanewater" ; Cn1cc(Cc2cc(F)ccc2OCC=O)c(-c2cc(Cl)nc(N)n2)n1 ; O=C1CNCCN1 ; Cn1cc(Cc2cc(F)ccc2OCCN2CCNC(=O)C2)c(-c2cc(Cl)nc(N)n2)n1]

Example 171 was prepared in a similar fashion to that described for Example 88 in Scheme AB using the appropriate aldehyde in Step 1 and the amine in Step 8.

### Preparation of 2-allyloxy-5-fluoro-benzaldehyde

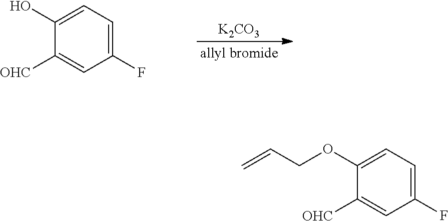

To a solution of 5-fluoro-2-hydroxy-benzaldehyde (10 g, 71 mmol, 1 eq) in MeCN (150 mL) was added K₂CO₃ (15 g, 107 mmol, 1.5 eq) and 3-bromoprop-1-ene (11.2 g, 92.8 mmol, 1.3 eq). The mixture was stirred at 20° C. for 12 h under N₂. The mixture was then heated to 60° C. for another 5 h under N₂. The reaction mixture was filtered, and the filtrate was poured into H₂O (250 mL). The mixture was extracted with ethyl acetate (100 mL*3). The organic phase was washed with brine (150 mL), dried over anhydrous Na₂SO₄, and filtered. The filtrate was concentrated. The residue was purified by flash silica gel chromatography (ISCO®; 120 g SepaFlash@ Silica Flash Column, Eluent of 0-6% Ethyl acetate/Petroleum ether gradient @ 100 mL/min) to furnish 2-allyloxy-5-fluoro-benzaldehyde.

Example 171: ¹H NMR: (400 MHz, DMSO-d6) δ 7.77 (br s, 1H), 7.47 (s, 1H), 7.14 (s, 2H), 7.08 (br d, J=8.7 Hz, 1H), 6.99 (s, 1H), 6.98-6.93 (m, 2H), 4.22 (s, 2H), 4.05 (t, J=5.3 Hz, 2H), 3.80 (s, 3H), 3.09 (br s, 2H), 3.02 (s, 2H), 2.72 (br t, J=5.4 Hz, 2H), 2.62-2.59 (m, 2H); LCMS: (MH+) 460.2.

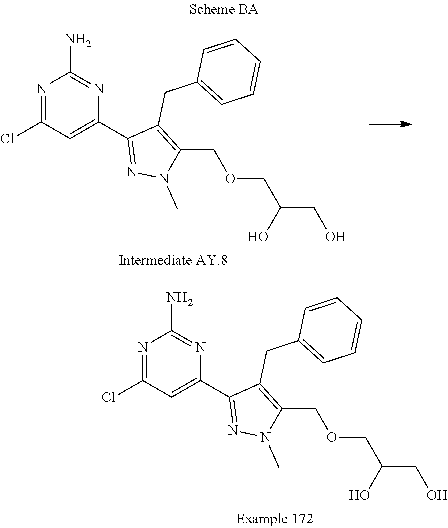 [SMILES: Cn1nc(-c2cc(Cl)nc(N)n2)c(Cc2ccccc2)c1COCC(O)CO ; Cn1nc(-c2cc(Cl)nc(N)n2)c(Cc2ccccc2)c1COCC(O)CO]

Example 172: ¹H NMR: (400 MHz, methanol-d4) δ 7.21-7.14 (m, 4H), 7.10 (s, 2H), 4.56 (s, 2H), 4.39 (s, 2H), 3.96 (s, 3H), 3.74-3.68 (m, 1H), 3.53-3.40 (m, 3H), 3.39-3.33 (m, 1H); LCMS: (MH+) 404.1.

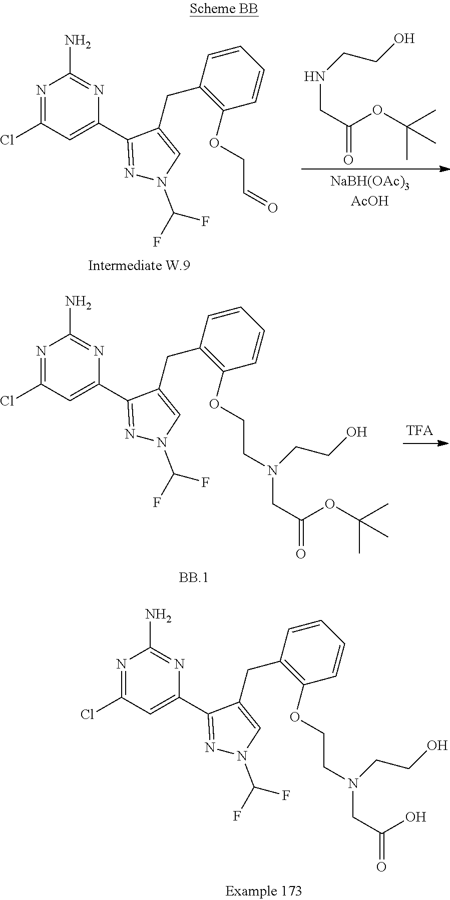 [SMILES: Nc1nc(Cl)cc(-c2nn(C(F)F)cc2Cc2ccccc2OCC=O)n1 ; CC(C)(C)OC(=O)CNCCO ; CC(C)(C)OC(=O)CN(CCO)CCOc1ccccc1Cc1cn(C(F)F)nc1-c1cc(Cl)nc(N)n1 ; Nc1nc(Cl)cc(-c2nn(C(F)F)cc2Cc2ccccc2OCCN(CCO)CC(=O)O)n1]

### Step 1

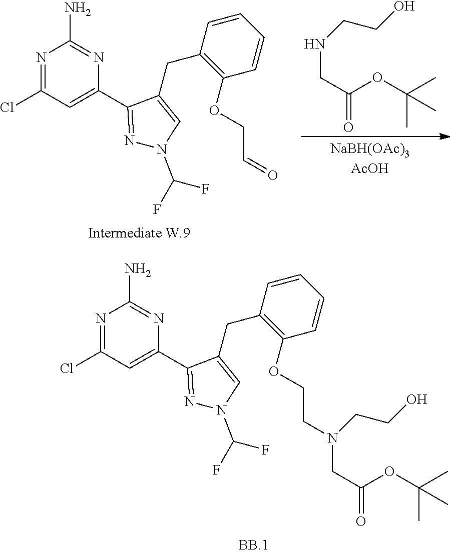 [SMILES: Nc1nc(Cl)cc(-c2nn(C(F)F)cc2Cc2ccccc2OCC=O)n1 ; CC(C)(C)OC(=O)CNCCO ; CC(C)(C)OC(=O)CN(CCO)CCOc1ccccc1Cc1cn(C(F)F)nc1-c1cc(Cl)nc(N)n1]

To a solution of 2-[2-[[3-(2-amino-6-chloro-pyrimidin-4-yl)-1-(difluoromethyl) pyrazol-4-yl]methyl]phenoxy]acetaldehyde W.9 (200 mg, 0.508 mmol, 1 eq) in DCE (3 mL) was added tert-butyl 2-(2-hydroxyethylamino)acetate (134 mg, 0.762 mmol, 1.5 eq) and HOAc (31 mg, 0.508 mmol, 29 μL, 1 eq). The reaction mixture was stirred at 20° C. for 3 h, and then NaBH(OAc)₃ (323 mg, 1.52 mmol, 3 eq) was added. The reaction mixture was stirred at 20° C. for 12 hr. The reaction mixture was quenched with saturated aqueous NaHCO₃ (40 mL). The mixture was extracted with EtOAc (20 mL*3). The combined organic layer was washed with brine (30 mL), dried over Na₂SO₄, and filtered. The filtrate was concentrated under reduced pressure. The residue was purified by flash silica gel chromatography (ISCO®; 4 g SepaFlash® Silica Flash Column, Eluent of 0-45% Ethyl acetate/Petroleum ether gradient at 40 mL/min) to furnish tert-butyl 2-[2-[2-[[3-(2-amino-6-chloro-pyrimidin-4-yl)-1-(difluoromethyl) pyrazol-4-yl]methyl] phenoxy]ethyl-(2-hydroxyethyl)amino]acetate as a colorless oil.

### Step 2

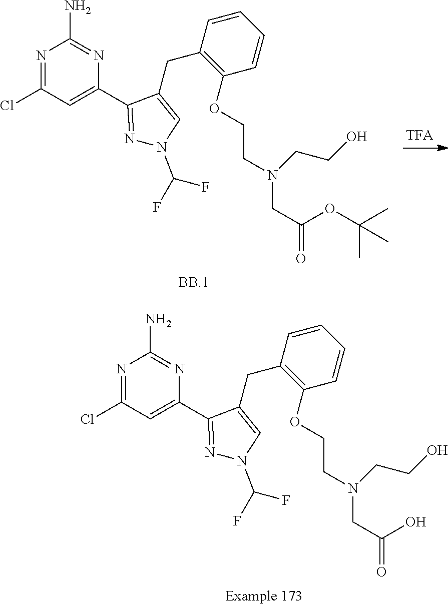 [SMILES: CC(C)(C)OC(=O)CN(CCO)CCOc1ccccc1Cc1cn(C(F)F)nc1-c1cc(Cl)nc(N)n1 ; Nc1nc(Cl)cc(-c2nn(C(F)F)cc2Cc2ccccc2OCCN(CCO)CC(=O)O)n1]

To a solution of tert-butyl 2-[2-[2-[[3-(2-amino-6-chloro-pyrimidin-4-yl)-1-(difluoromethyl)pyrazol-4-yl]methyl]phenoxy]ethyl-(2-hydroxyethyl)amino]acetate (50 mg, 0.090 mmol, 1 eq) in DCM (2 mL) was added TFA (1 mL). The reaction mixture was stirred at 20° C. for 3 hr. Additional TFA (1 mL) was added, and the mixture was stirred at 20° C. for 2.5 hr. The reaction mixture was quenched with saturated aqueous NaHCO₃ (30 mL). The mixture was extracted with EtOAc (20 mL*3). The combined organic layer was washed with brine (20 mL), dried over Na₂SO₄, and filtered. The filtrate was concentrated under reduced pressure. The residue was purified by prep-HPLC (column: Phenomenex Luna C18 100*30 mm*5 um; mobile phase: [water (0.2% FA)-ACN]; B %: 28%-58%, 9 min) to furnish 2-[2-[2-[[3-(2-amino-6-chloro-pyrimidin-4-yl)-1-(difluoromethyl)pyrazol-4-yl]methyl]phenoxy]ethyl-(2-hydroxyethyl)amino]acetic acid Example 173.

Example 173: ¹H NMR: (400 MHz, CHLOROFORM-d) δ 7.33-7.29 (m, 1H), 7.24-6.78 (m, 6H), 6.27-5.77 (m, 2H), 4.29-4.12 (m, 4H), 3.73-3.52 (m, 4H), 3.39-3.21 (m, 2H), 3.08-2.89 (m, 2H)′ LCMS: (MH+) 497.2.

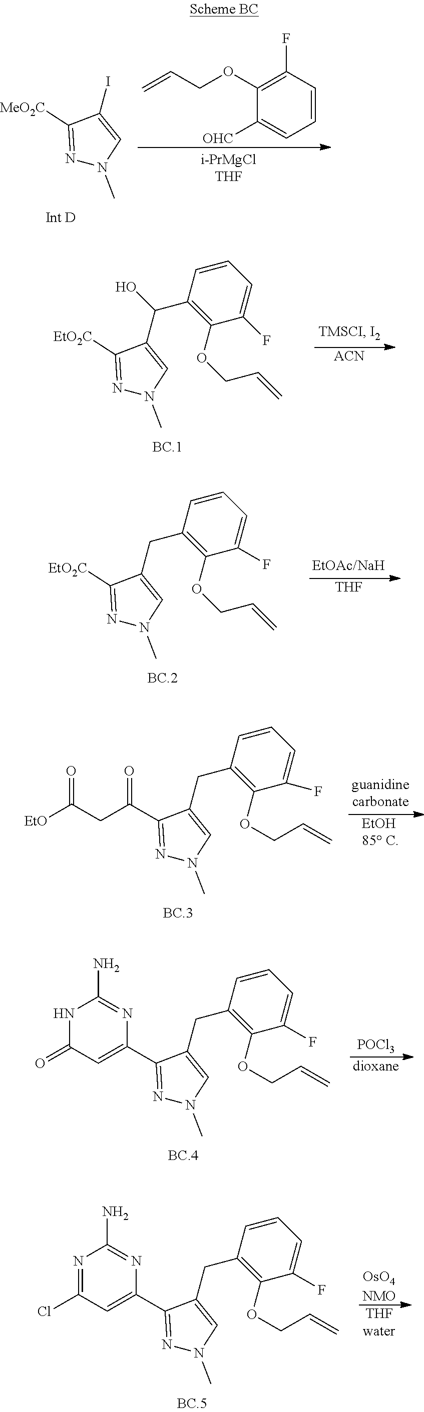

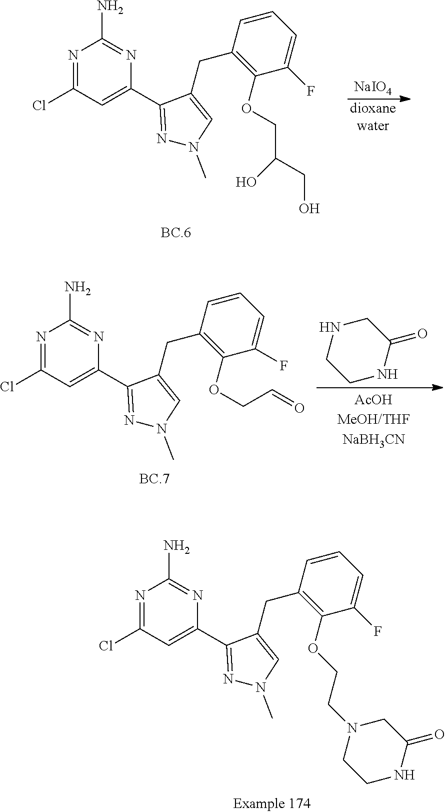

Example 174 was prepared in a similar fashion to that described for Example 88 in Scheme AB using the appropriate aldehyde in Step 1 and the amine in Step 8.

### Preparation of 2-allyloxy-3-fluoro-benzaldehyde

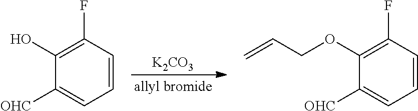

To a solution of 3-fluoro-2-hydroxy-benzaldehyde (10 g, 71 mmol, 1 eq) in MeCN (200 mL) was added K₂CO₃ (14.8 g, 107 mmol, 1.5 eq) and allyl bromide (11.2 g, 92.8 mmol, 1.3 eq). The mixture was stirred at 60° C. for 12 hr under N₂. The reaction mixture was quenched with saturated aqueous NH₄Cl (400 mL). The mixture was extracted with EtOAc (200 mL*3). The combined organic layer was washed with brine (150 mL), dried over Na₂SO₄, and filtered. The filtrate was concentrated under reduced pressure. The residue was purified by flash silica gel chromatography (ISCO®; 80 g SepaFlash@ Silica Flash Column, Eluent of 0-10% Ethyl acetate/Petroleum ether gradient at 100 mL/min) to furnish 2-allyloxy-3-fluoro-benzaldehyde as a colorless oil.

Example 174: ¹H NMR: (400 MHz, DMSO-d₆) δ 7.72 (br s, 1H), 7.39 (s, 1H), 7.13-6.96 (m, 6H), 4.32 (s, 2H), 4.07 (t, J=5.3 Hz, 2H), 3.82 (s, 3H), 3.06 (br s, 2H), 2.98 (s, 2H), 2.65 (br t, J=5.6 Hz, 2H), 2.61-2.57 (m, 2H); LCMS: (MH+) 460.2.

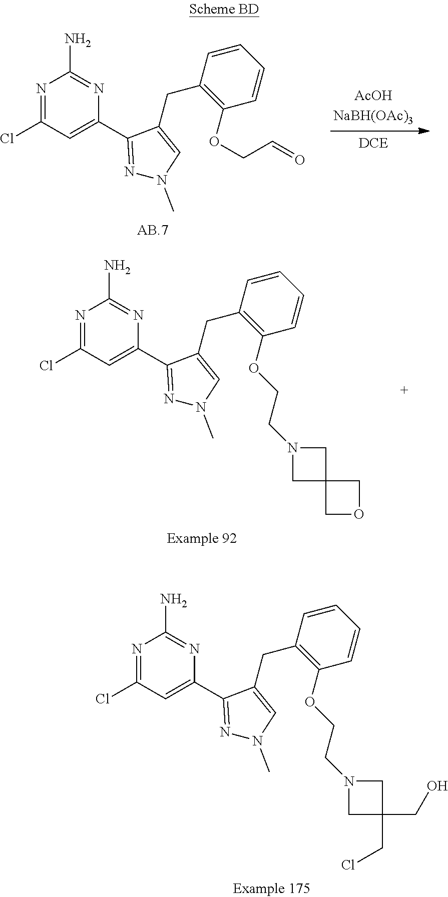 [SMILES: Cn1cc(Cc2ccccc2OCC=O)c(-c2cc(Cl)nc(N)n2)n1 ; Cn1cc(Cc2ccccc2OCCN2CC3(COC3)C2)c(-c2cc(Cl)nc(N)n2)n1 ; Cn1cc(Cc2ccccc2OCCN2CC(CO)(CCl)C2)c(-c2cc(Cl)nc(N)n2)n1]

During the preparation of Example 92, it was observed that Example 175 was also formed after workup of the reaction mixture. The residue was purified by pre-HPLC (column: Waters Xbridge Prep OBD C18 150*40 mm*10 um; mobile phase: [water (10 mM NH₄HCO₃)-ACN]; B %: 30%-50%, 8 min) to furnish [1-[2-[2-[[3-(2-amino-6-chloro-pyrimidin-4-yl)-1-methyl-pyrazol-4-yl]methyl] phenoxy]ethyl]-3-(chloromethyl)azetidin-3-yl]methanol Example 175.

Example 175: ¹H NMR: (CHLOROFORM-d, 400 MHz) δ 7.23-7.16 (m, 2H), 7.12 (d, J=6.4 Hz, 1H), 6.93-6.86 (m, 2H), 6.83 (d, J=8.1 Hz, 1H), 5.35 (br s, 2H), 4.17 (s, 2H), 3.99 (t, J=5.0 Hz, 2H), 3.87 (s, 3H), 3.71 (d, J=6.6 Hz, 4H), 3.12-3.00 (m, 4H), 2.81 (t, J=4.9 Hz, 2H); LCMS: (MH+) 477.2.

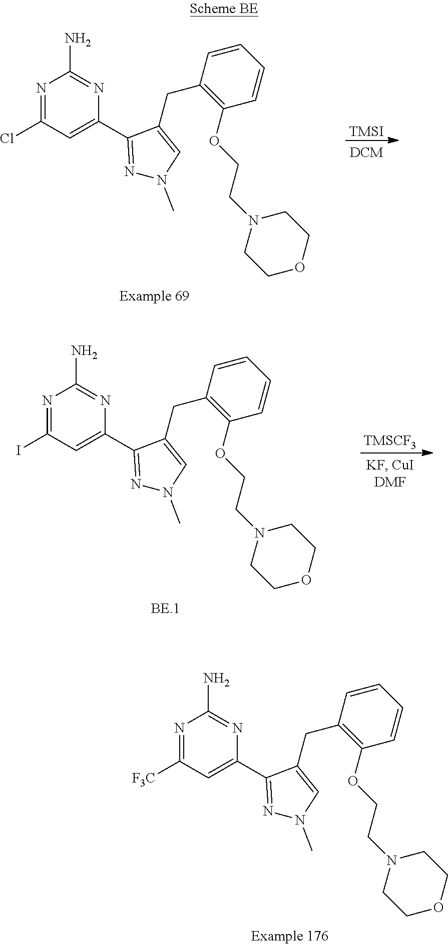

### Step 1

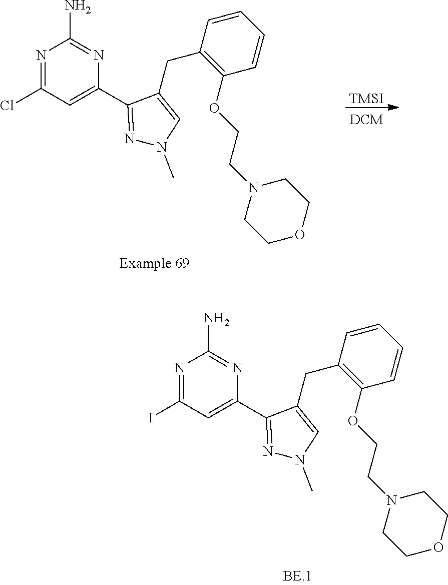 [SMILES: "TMSI" ; Cn1cc(Cc2ccccc2OCCN2CCOCC2)c(-c2cc(Cl)nc(N)n2)n1 ; Cn1cc(Cc2ccccc2OCCN2CCOCC2)c(-c2cc(I)nc(N)n2)n1]

A solution of 4-chloro-6-[1-methyl-4-[[2-(2-morpholinoethoxy)phenyl]methyl] pyrazol-3-yl]pyrimidin-2-amine Example 69 (50 mg, 0.12 mmol, 1 eq) in DCM (3 mL) was added TMSI (117 mg, 0.583 mmol, 79 μL, 5 eq). The mixture was stirred at 20° C. for 27 hr. The reaction mixture was quenched with saturated aqueous NaHCO₃ (50 mL). The mixture was extracted with EtOAc (80 mL*3). The combined organic layer was washed with saturated aqueous Na₂SO₃ (50 mL), brine (30 mL), and dried over Na₂SO₄. The filtrate was concentrated under reduced pressure. The residue was purified by flash silica gel chromatography (ISCO®; 4 g SepaFlash@ Silica Flash Column, Eluent of 0-100% Ethyl acetate/Petroleum ether gradient of 45 mL/min) to furnish 4-iodo-6-[l-methyl-4-[[2-(2-morpholinoethoxy)phenyl]methyl]pyrazol-3-yl]pyrimidin-2-amine as a white solid.

### Step 2

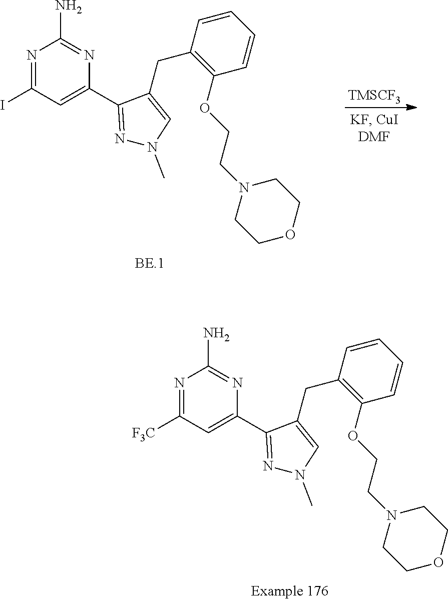 [SMILES: "TMSCF3" ; Cn1cc(Cc2ccccc2OCCN2CCOCC2)c(-c2cc(I)nc(N)n2)n1 ; Cn1cc(Cc2ccccc2OCCN2CCOCC2)c(-c2cc(C(F)(F)F)nc(N)n2)n1]

4-iodo-6-[1-methyl-4-[[2-(2-morpholinoethoxy)phenyl]methyl]pyrazol-3-yl]pyrimidin-2-amine (34 mg, 0.065 mmol, 1 eq), trimethyl(trifluoromethyl)silane (23 mg, 0.163 mmol, 2.5 eq), KF (19 mg, 0.327 mmol, 7 μL, 5 eq) and CuI (37 mg, 0.20 mmol, 3 eq) were taken up into a microwave tube in DMF (1 mL). The sealed tube was heated at 100° C. for 0.5 hr under microwave irradiation. The reaction mixture was diluted with H₂O (20 mL) and extracted with EtOAc (20 mL*3). The combined organic layers were washed with brine (30 mL), dried over Na₂SO₄, and filtered. The filtrate was concentrated under reduced pressure. The residue was purified by preparative-HPLC (column: Waters Xbridge BEH C18 100*30 mm*10 um; mobile phase: [water (10 mM NH₄HCO₃)-ACN]; B %: 30%-55%, 8 min) to furnish 4-[1-methyl-4-[[2-(2-morpholinoethoxy)phenyl]methyl] pyrazol-3-yl]-6-(trifluoromethyl)pyrimidin-2-amine Example 176.

Example 176: ¹H NMR: (400 MHz, CHLOROFORM-d) δ 7.53 (s, 1H), 7.23-7.05 (m, 3H), 6.93-6.84 (m, 2H), 5.37-5.23 (m, 2H), 4.25 (s, 2H), 4.13 (t, J=5.6 Hz, 2H), 3.91 (s, 3H), 3.70-3.64 (m, 4H), 2.77 (t, J=5.6 Hz, 2H), 2.56-2.49 (m, 4H); LCMS: (MH+) 463.2.

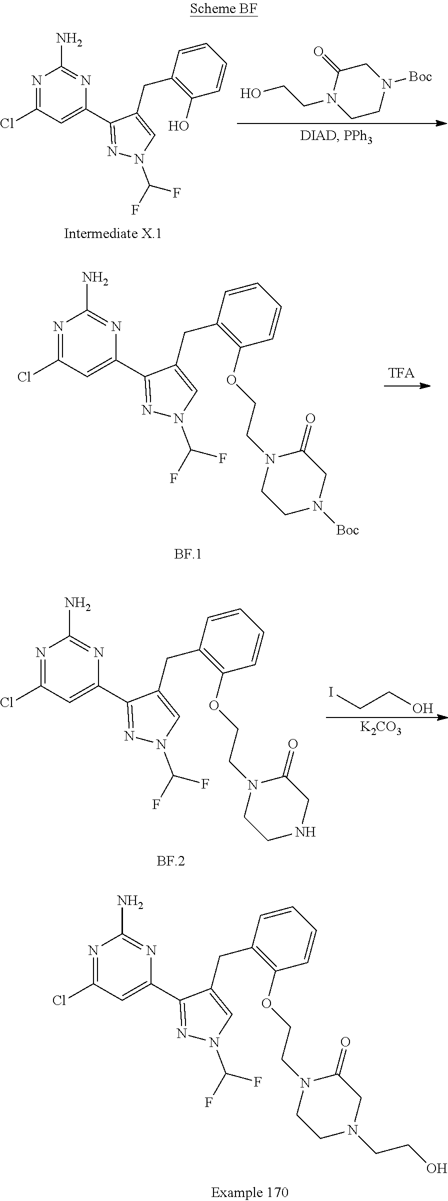

### Step 1

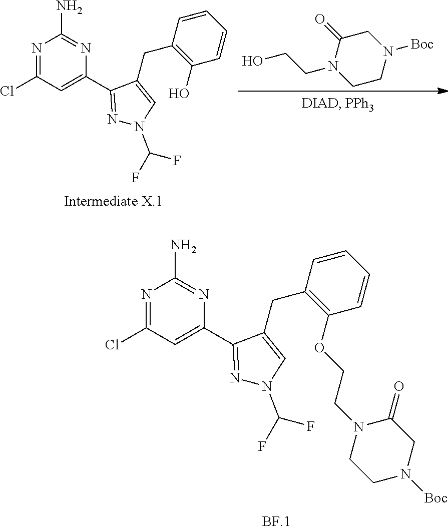

To a solution of 2-[[3-(2-amino-6-chloro-pyrimidin-4-yl)-1-(difluoromethyl) pyrazol-4-yl]methyl]phenol Intermediate X.1 (320 mg, 0.910 mmol, 1 eq), tert-butyl 4-(2-hydroxyethyl)-3-oxo-piperazine-1-carboxylate (289 mg, 1.18 mmol, 1.3 eq) and triphenylphosphine (358 mg, 1.36 mmol, 1.5 eq) in THF (2 mL) was added a solution of DIAD (276 mg, 1.36 mmol, 0.27 mL, 1.5 eq) in THF (0.5 mL) at 0° C. The reaction mixture was degassed and purged with N₂ for three times and stirred at 80° C. for 15 hr under N₂. The reaction mixture was diluted with H₂O (30 mL) and extracted with EtOAc (20 mL*3). The combined organic layers were washed with brine (20 mL), dried over Na₂SO₄, and filtered. The filtrate was concentrated under reduced pressure. The residue was purified by flash silica gel chromatography (ISCO®; 4 g SepaFlash® Silica Flash Column, Eluent of 0-45% Ethyl acetate/Petroleum ether gradient of 40 mL/min to furnish a residue. The residue was further purified by preparative-TLC (Petroleum ether: Ethyl acetate=0:1) to furnish tert-butyl 4-[2-[2-[[3-(2-amino-6-chloro-pyrimidin-4-yl)-1-(difluoromethyl)pyrazol-4-yl]methyl]phenoxy]ethyl]-3-oxo-piperazine-1-carboxylate.

### Step 2

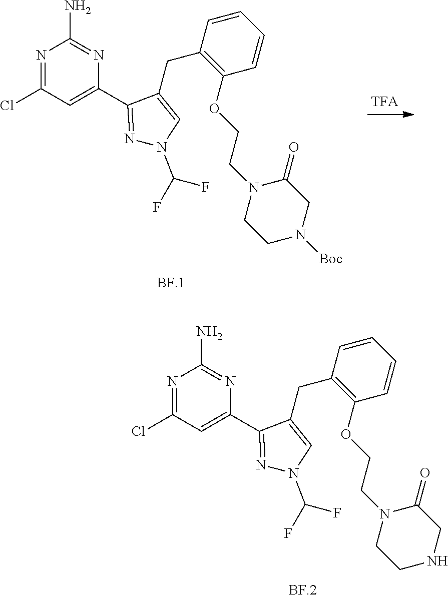 [SMILES: CC(C)(C)OC(=O)N1CCN(CCOc2ccccc2Cc2cn(C(F)F)nc2-c2cc(Cl)nc(N)n2)C(=O)C1 ; Nc1nc(Cl)cc(-c2nn(C(F)F)cc2Cc2ccccc2OCCN2CCNCC2=O)n1]

A mixture of tert-butyl 4-[2-[2-[[3-(2-amino-6-chloro-pyrimidin-4-yl)-1-(difluoromethyl)pyrazol-4-yl]methyl]phenoxy]ethyl]-3-oxo-piperazine-1-carboxylate (120 mg, 0.208 mmol, 1 eq) in DCM (2 mL) and TFA (0.4 mL) was stirred at 20° C. for 1 hr. The reaction mixture was quenched with saturated aqueous NaHCO₃ (30 mL). The mixture was extracted with EtOAc (20 mL*3). The combined organic layer was washed with brine (20 mL), dried over Na₂SO₄, and filtered. The filtrate was concentrated under reduced pressure to give 1-[2-[2-[[3-(2-amino-6-chloro-pyrimidin-4-yl)-1-(difluoromethyl)pyrazol-4-yl]methyl]phenoxy]ethyl]piperazin-2-one.

### Step 3

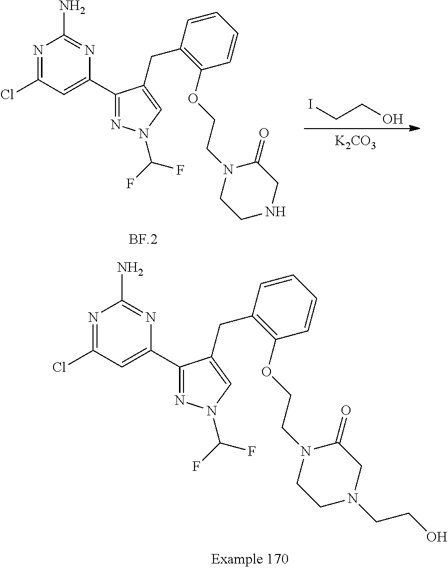

To a solution of 1-[2-[2-[[3-(2-amino-6-chloro-pyrimidin-4-yl)-1-(difluoromethyl) pyrazol-4-yl]methyl]phenoxy]ethyl]piperazin-2-one (80 mg, 0.17 mmol, 1 eq) in THF (2 mL) was added 2-iodoethanol (144 mg, 0.837 mmol, 65 μL, 5 eq) and K₂CO₃ (93 mg, 0.67 mmol, 4 eq). The reaction mixture was stirred at 65° C. for 20 hr. The reaction mixture was filtered and the filtrate was concentrated. The residue was purified by preparative-HPLC (column: Phenomenex Luna C18 200*40 mm*10 um; mobile phase: [water (0.2% FA)-ACN]; B %: 20%-50%, 8 min) to furnish 1-[2-[2-[[3-(2-amino-6-chloro-pyrimidin-4-yl)-1-(difluoromethyl)pyrazol-4-yl] methyl]phenoxy]ethyl]-4-(2-hydroxyethyl)piperazin-2-one Example 170.

Example 170: ¹H NMR: (400 MHz, CHLOROFORM-d) δ 7.33-7.30 (m, 2H), 7.26-7.23 (m, 0.8H), 7.18-7.12 (m, 2H), 7.02-7.00 (m, 0.2H), 6.97-6.88 (m, 2H), 5.40-5.32 (m, 2H), 4.24-4.21 (m, 2H), 4.21-4.16 (m, 2H), 3.73-3.68 (m, 2H), 3.64-3.59 (m, 2H), 3.31-3.26 (m, 2H), 3.17-3.14 (m, 2H), 2.52 (td, J=5.3, 12.5 Hz, 4H); LCMS: (MH+) 522.2.

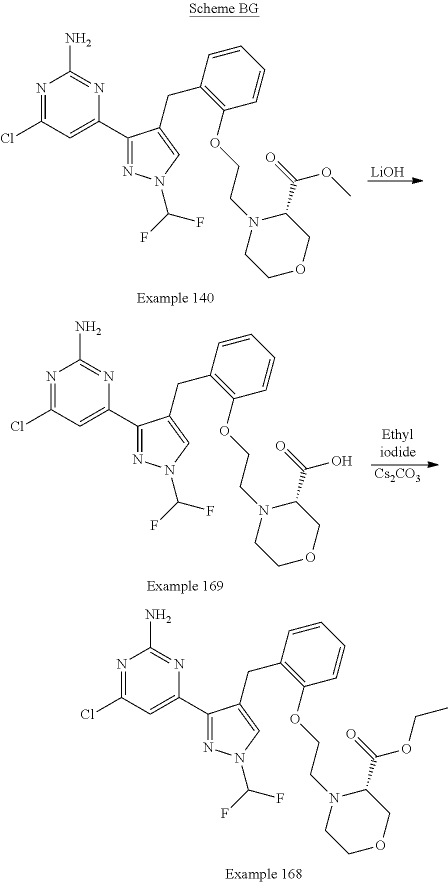

### Step 1

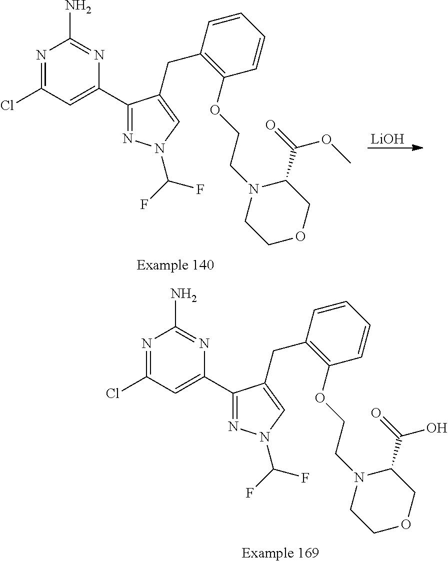

To a solution of methyl (3S)-4-[2-[2-[[3-(2-amino-6-chloro-pyrimidin-4-yl)-1-(difluoromethyl)pyrazol-4-yl]methyl]phenoxy]ethyl]morpholine-3-carboxylate Example 140 (100 mg, 0.191 mmol, 1 eq) in dioxane (2 mL) and H₂O (0.4 mL) was added LiOH·H₂O (120 mg, 2.87 mmol, 15 eq). Then the reaction mixture was stirred at 60° C. for 12 hr under N₂. The reaction mixture was filtered, and the filtrate was concentrated. The residue was purified by preparative-HPLC (column: Phenomenex Gemini-NX 150*30 mm*5 um; mobile phase: [water (10 mM NH₄HCO₃)-ACN]; B %: 15%-35%, 8 min) to furnish a residue. The residue was further purified by SFC (column: DAICEL CHIRALCEL OX (250 mm*30 mm, 10 um); mobile phase: [0.1% NH₃H₂O MEOH]; B %: 60%-60%, min) to furnish (3S)-4-[2-[2-[[3-(2-amino-6-chloro-pyrimidin-4-yl)-1-(difluoromethyl)pyrazol-4-yl]methyl]phenoxy]ethyl]morpholine-3-carboxylic acid Example 169.

Example 169: ¹H NMR: (400 MHz, CHLOROFORM-d) δ 7.46-7.29 (m, 1H), 7.26-7.09 (m, 2H), 7.07-6.98 (m, 1H), 6.96-6.82 (m, 2H), 6.67-6.47 (m, 1H), 4.45-4.21 (m, 2H), 4.18-4.06 (m, 1H), 4.02-3.66 (m, 5H), 3.61-3.48 (m, 1H), 3.43-3.19 (m, 2H), 3.05-2.90 (m, 1H), 2.66-2.51 (m, 1H); LCMS: (MH+) 509.0.

### Step 2

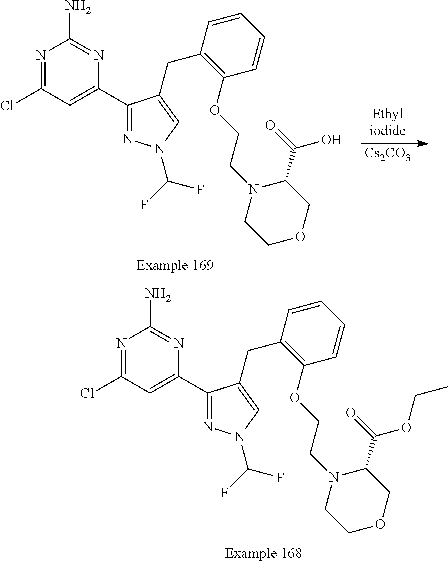

To a solution of (3S)-4-[2-[2-[[3-(2-amino-6-chloro-pyrimidin-4-yl)-1-(difluoromethyl) pyrazol-4-yl]methyl]phenoxy]ethyl]morpholine-3-carboxylic acid Example 169 (100 mg, 0.197 mmol, 1 eq) in DMF (2 mL) was added Cs₂CO₃ (96 mg, 0.29 mmol, 1.5 eq) and iodoethane (43 mg, 0.28 mmol, 22 μL, 1.4 eq) at 0° C. The mixture was stirred at 0° C. for 1 hr and then at 20° C. for 12 hr. The reaction mixture was filtered, and the filtrate was directly purified. The residue was purified by preparative-HPLC (column: Phenomenex Gemini-NX C18 75*30 mm*3 um; mobile phase: [water (10 mM NH4HCO3)-ACN]; B %: 50%-70%, 8 min) to furnish a residue. The residue was further purified by SFC (column: DAICEL CHIRALCEL OJ (250 mm*30 mm, 10 um); mobile phase: [0.1% NH3H2O MEOH]; B %: 35%-35%, min) to furnish ethyl (3S)-4-[2-[2-[[3-(2-amino-6-chloro-pyrimidin-4-yl)-1-(difluoromethyl)pyrazol-4-yl]methyl]phenoxy]ethyl]morpholine-3-carboxylate Example 168.

Example 168: ¹H NMR: (400 MHz, CHLOROFORM-d) δ 7.53-7.48 (m, 1H), 7.35-7.28 (m, 1H), 7.24-6.97 (m, 3H), 6.92-6.85 (m, 2H), 5.41-5.25 (m, 2H), 4.34-4.25 (m, 1H), 4.24-4.06 (m, 5H), 3.90-3.83 (m, 1H), 3.82-3.75 (m, 1H), 3.75-3.67 (m, 1H), 3.66-3.57 (m, 1H), 3.37 (dd, J=3.7, 5.1 Hz, 1H), 3.25-3.16 (m, 1H), 3.08-2.99 (m, 1H), 2.98-2.88 (m, 1H), 2.50 (ddd, J=3.1, 5.9, 11.7 Hz, 1H), 1.25 (t, J=7.2 Hz, 3H); LCMS: (MH+) 537.1.

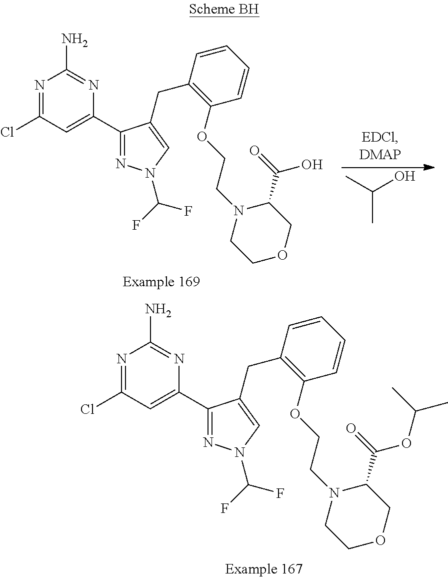

To a stirred solution of (3S)-4-[2-[2-[[3-(2-amino-6-chloro-pyrimidin-4-yl)-1-(difluoromethyl) pyrazol-4-yl]methyl]phenoxy]ethyl]morpholine-3-carboxylic acid Example 169 (150 mg, 0.295 mmol, 1 eq) in DCM (2 mL) was added DMAP (72 mg, 0.59 mmol, 2 eq), EDCI (113 mg, 0.59 mmol, 2 eq), and i-PrOH (354 mg, 5.90 mmol, 0.45 ml, 20 eq). The mixture was stirred at 20° C. for 12 hr. The reaction mixture was diluted with H₂O (50 mL) and extracted with EtOAc (30 mL*3). The combined organic layers were washed with brine (50 mL), dried over Na₂SO₄, and filtered. The filtrate was concentrated under reduced pressure. The residue was purified by preparative-HPLC (column: Phenomenex Gemini-NX C18 75*30 mm*3 um; mobile phase: [water (10 mM NH4HCO3)-ACN]; B %: 50%-70%, 8 min) to furnish a residue. The residue was further purified by SFC (column: DAICEL CHIRALCEL OD (250 mm*30 mm, 10 um); mobile phase: [0.1% NH3H2O IPA]; B %: 40%-40%, min) to furnish isopropyl (3S)-4-[2-[2-[[3-(2-amino-6-chloro-pyrimidin-4-yl)-1-(difluoromethyl)pyrazol-4-yl]methyl]phenoxy]ethyl]morpholine-3-carboxylate Example 167.

Example 167: ¹H NMR: (400 MHz, CHLOROFORM-d) δ 7.53-7.49 (m, 1H), 7.32-7.27 (m, 1H), 7.24-7.00 (m, 3H), 6.91-6.85 (m, 2H), 5.41-5.27 (m, 2H), 5.11-5.00 (m, 1H), 4.34-4.27 (m, 1H), 4.22-4.06 (m, 3H), 3.88-3.75 (m, 2H), 3.72-3.58 (m, 2H), 3.32 (dd, J=3.7, 5.5 Hz, 1H), 3.24-3.16 (m, 1H), 3.04 (td, J=5.1, 14.1 Hz, 1H), 2.94-2.85 (m, 1H), 2.49 (ddd, J=3.2, 6.3, 11.6 Hz, 1H), 1.23 (dd, J=4.4, 6.2 Hz, 6H); LCMS: (MH+) 551.1.

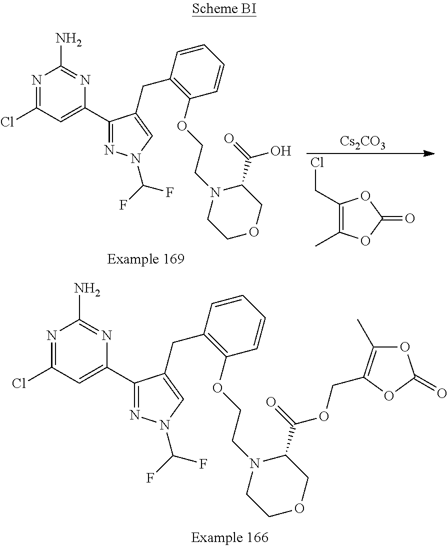

To a solution of (3S)-4-[2-[2-[[3-(2-amino-6-chloro-pyrimidin-4-yl)-1-(difluoromethyl) pyrazol-4-yl]methyl]phenoxy]ethyl]morpholine-3-carboxylic acid Example 169 (100 mg, 0.197 mmol, 1 eq) in DMF (2 mL) was added Cs₂CO₃ (96 mg, 0.29 mmol, 1.5 eq) and 4-(chloromethyl)-5-methyl-1,3-dioxol-2-one (41 mg, 0.28 mmol, 1.4 eq) at 0° C. After stirring at 0° C. for 1 hr, the mixture was stirred an additional 12 hr at 20° C. The reaction mixture was filtered, and the filtrate was directly purified. The residue was purified by preparative-HPLC (column: Waters Xbridge BEH C18 100*30 mm*10 um; mobile phase: [water (10 mM NH₄HCO₃)-ACN]; B %: 40%-70%, 8 min) to furnish a residue. The material was further purified by SFC (column: DAICEL CHIRALCEL OD (250 mm*50 mm, 10 um); mobile phase: [Neu-IPA]; B %: 55%-55%, min) to furnish (5-methyl-2-oxo-1,3-dioxol-4-yl)methyl (3S)-4-[2-[2-[[3-(2-amino-6-chloro-pyrimidin-4-yl)-1-(difluoromethyl)pyrazol-4-yl]methyl]phenoxy]ethyl]morpholine-3-carboxylate Example 166.

Example 166: ¹H NMR: (400 MHz, CHLOROFORM-d) δ 7.47 (s, 1H), 7.33-7.28 (m, 1H), 7.25-7.10 (m, 3H), 7.03-7.00 (m, 1H), 6.94-6.84 (m, 2H), 5.32 (br s, 2H), 4.94-4.76 (m, 2H), 4.31-4.17 (m, 2H), 4.15-4.03 (m, 2H), 3.91 (dd, J=4.3, 11.2 Hz, 1H), 3.78-3.67 (m, 2H), 3.64-3.54 (m, 1H), 3.46 (t, J=3.9 Hz, 1H), 3.20 (ddd, J=3.3, 8.5, 11.8 Hz, 1H), 3.07-2.91 (m, 2H), 2.52 (td, J=3.7, 11.9 Hz, 1H), 2.13 (s, 3H); LCMS: (MH+) 621.1.

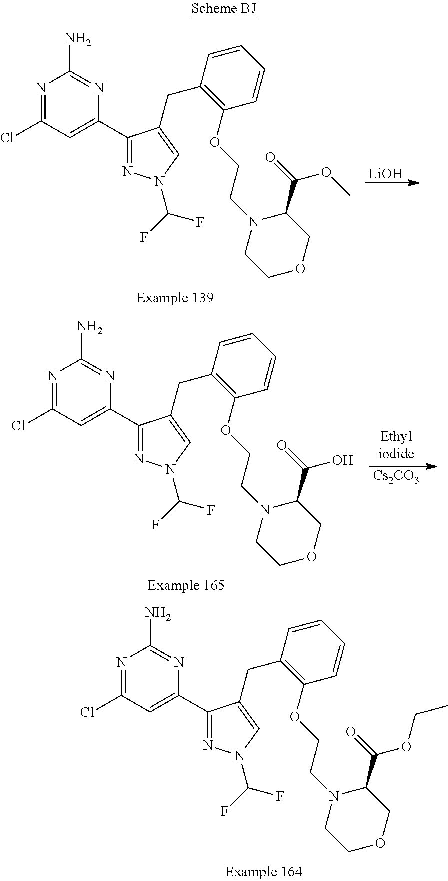

### Step 1

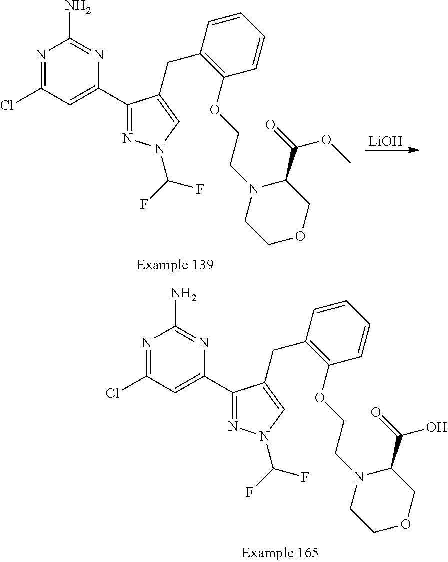

Example 165 was prepared from Example 139 using conditions like those outlined in Scheme BG.

Example 165: ¹H NMR: (400 MHz, CHLOROFORM-d) δ 7.50-7.35 (m, 1H), 7.30-7.03 (m, 4H), 6.99-6.89 (m, 2H), 6.73-6.56 (m, 1H), 4.50-4.27 (m, 2H), 4.22-4.11 (m, 1H), 4.05-3.72 (m, 5H), 3.60 (dd, J=3.7, 6.4 Hz, 1H), 3.46-3.27 (m, 2H), 3.09-2.98 (m, 1H), 2.69-2.59 (m, 1H); LCMS: (MH+) 509.1.

### Step 2

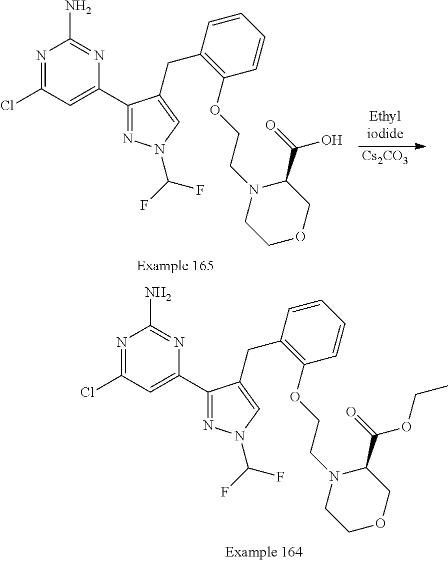

Example 164 was prepared from Example 165 using conditions like that described in Scheme BG.

Example 164: ¹H NMR: (400 MHz, CHLOROFORM-d) δ 7.49 (s, 1H), 7.32-7.26 (m, 1H), 7.23-7.14 (m, 1H), 7.09 (dd, J=1.7, 7.6 Hz, 1H), 7.02-6.99 (m, 1H), 6.91-6.85 (m, 2H), 5.38-5.26 (m, 2H), 4.32-4.25 (m, 1H), 4.22-4.05 (m, 5H), 3.89-3.82 (m, 1H), 3.81-3.74 (m, 1H), 3.74-3.66 (m, 1H), 3.65-3.57 (m, 1H), 3.36 (dd, J=3.5, 5.3 Hz, 1H), 3.23-3.15 (m, 1H), 3.08-2.97 (m, 1H), 2.97-2.87 (m, 1H), 2.55-2.43 (m, 1H), 1.23 (t, J=7.2 Hz, 3H); LCMS: (MH+) 537.1.

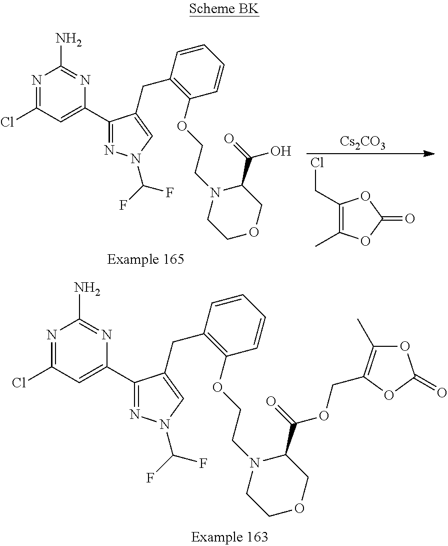

Example 163 was prepared from Example 165 using conditions like that outlined in Scheme BI.

Example 163: ¹H NMR: (400 MHz, CHLOROFORM-d) δ 7.47 (s, 1H), 7.34-7.28 (m, 1H), 7.25-6.99 (m, 3H), 6.95-6.84 (m, 2H), 5.38-5.26 (m, 2H), 4.93-4.77 (m, 2H), 4.31-4.16 (m, 2H), 4.15-4.04 (m, 2H), 3.91 (dd, J=4.2, 11.2 Hz, 1H), 3.77-3.68 (m, 2H), 3.64-3.55 (m, 1H), 3.46 (t, J=3.7 Hz, 1H), 3.20 (ddd, J=3.3, 8.5, 11.8 Hz, 1H), 3.06-2.92 (m, 2H), 2.56-2.48 (m, 1H), 2.13 (s, 3H); LCMS: (MH+) 621.0.

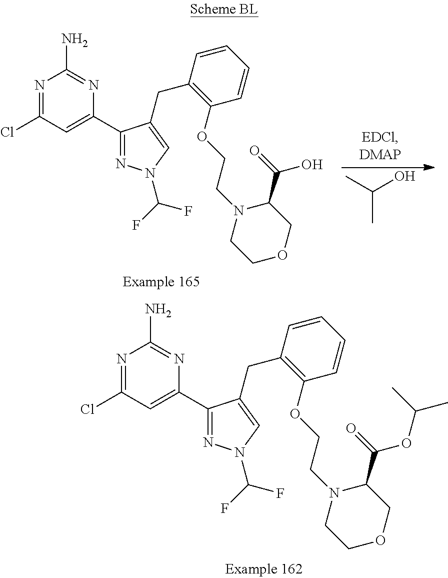

Example 162 was prepared from Example 165 using conditions like that described in Scheme BH.

Example 162: ¹H NMR: (400 MHz, CHLOROFORM-d) δ 7.44 (s, 1H), 7.24 (s, 1H), 7.20 (s, 1H), 7.16-7.10 (m, 1H), 7.09 (s, 1H), 7.02 (dd, J=1.4, 7.6 Hz, 1H), 6.94 (s, 1H), 6.85-6.77 (m, 2H), 5.26 (br s, 2H), 4.98 (td, J=6.3, 12.5 Hz, 1H), 4.27-4.10 (m, 2H), 4.10-3.99 (m, 2H), 3.79-3.68 (m, 2H), 3.66-3.50 (m, 2H), 3.27-3.22 (m, 1H), 3.17-3.09 (m, 1H), 2.96 (td, J=5.1, 13.9 Hz, 1H), 2.87-2.77 (m, 1H), 2.42 (ddd, J=3.2, 6.3, 11.4 Hz, 1H), 1.16 (dd, J=4.1, 6.3 Hz, 6H); LCMS: (MH+) 551.1.

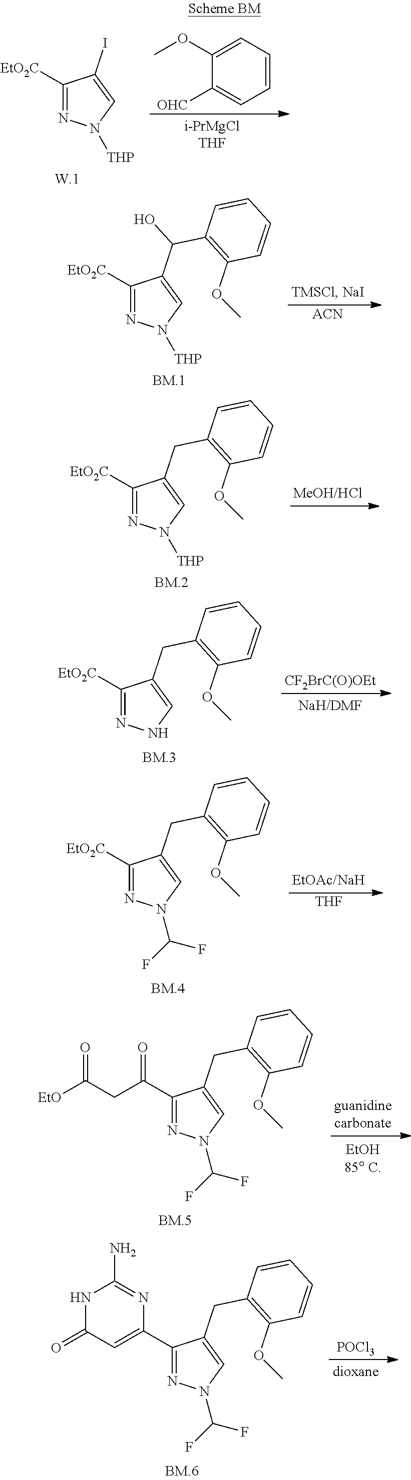

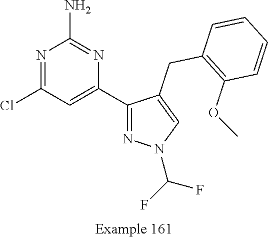 [SMILES: COc1ccccc1Cc1cn(C(F)F)nc1-c1cc(Cl)nc(N)n1]

Intermediate W.1 was converted into Example 161 using conditions like those described in Scheme W. The additional step (Step 3) is described below.

### Step 3

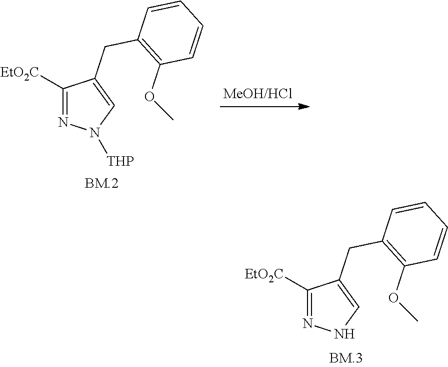

To a solution of ethyl 4-[(2-methoxyphenyl)methyl]-1-tetrahydropyran-2-yl-pyrazole-3-carboxylate (2.30 g, 6.68 mmol, 1 eq) in MeOH (8 mL) was added HCl/MeOH (4 M, 8 mL, 4.8 eq). The mixture was stirred at 20° C. for 0.5 h. The reaction mixture was concentrated under reduced pressure. The residue was purified by column chromatography (SiO₂, Petroleum ether: Ethyl acetate=7:3) to afford ethyl 4-[(2-methoxyphenyl)methyl]-1H-pyrazole-3-carboxylate.

Example 161: ¹H NMR: (DMSO-d6, 400 MHz) δ 7.92-7.63 (NM, 1H), 7.73 (s, 1H), 7.27 (s, 2H) 7.16-7.24 (m, 2H), 7.03 (s, 1H), 6.98 (d, J=8.11 Hz, 1H), 6.85-6.87 (m, 1H), 4.21 (s, 2H), 3.76 (s, 3H); LCMS: (MH+) 366.1.

The examples in Table 15 were prepared using conditions outlined in Scheme BM using the appropriate aldehyde in Step 1.

TABLE 15

| | | | | LCMS |
|---|---|---|---|---|
| Ex | Structure | Aldehyde | 1H NMR (400MHz) | (MH+) |
| 160 | 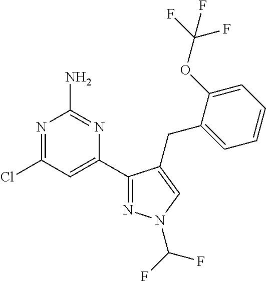 [SMILES: Nc1nc(Cl)cc(-c2nn(C(F)F)cc2Cc2ccccc2OC(F)(F)F)n1] | 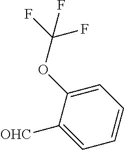 [SMILES: O=Cc1ccccc1OC(F)(F)F] | (DMSO-d6) δ 7.64-7.96 (m, 2H), 7.44 (d, J = 6.80 Hz, 1H), 7.17-7.40 (m, 5H), 7.04 (s, 1H), 4.36 (s, 2H). | 420.0 |
| 159 | 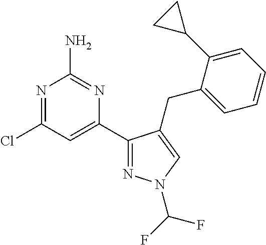 [SMILES: Nc1nc(Cl)cc(-c2nn(C(F)F)cc2Cc2ccccc2C2CC2)n1] | 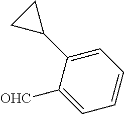 [SMILES: O=Cc1ccccc1C1CC1] | (DMSO-d6) δ 7.61-7.93 (m, 2H), 7.25 (s, 2H), 7.07-7.17 (m, 3H), 7.04 (s, 1H) 6.95 (d, J = 7.23 Hz, 1H), 4.43 (s, 2H), 1.83-1.95 (m, 1H), 0.74- 0.85 (m, 2H), 0.52-0.63 (m, 2H) | 376.0 |
| 158 | 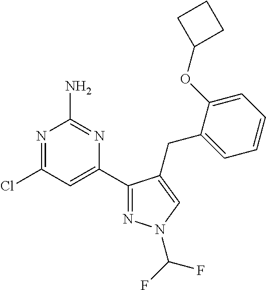 [SMILES: Nc1nc(Cl)cc(-c2nn(C(F)F)cc2Cc2ccccc2OC2CCC2)n1] | 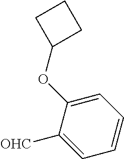 [SMILES: O=Cc1ccccc1OC1CCC1] | (DMSO-d6) δ 7.99-7.64 (m, 2H), 7.36-7.20 (m, 3H), 7.15 (dt, J = 1.7, 7.8 Hz, 1H), 7.03 (s, 1H), 6.88-6.77 (m, 2H), 4.73- 4.60 (m, 1H), 4.23 (s, 2H), 2.44-2.31 (m, 2H), 1.99-1.86 (m, 2H), 1.79- 1.55 (m, 2H) | 406.0 |
| 150 | 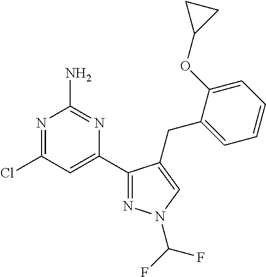 [SMILES: Nc1nc(Cl)cc(-c2nn(C(F)F)cc2Cc2ccccc2OC2CC2)n1] | 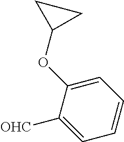 [SMILES: O=Cc1ccccc1OC1CC1] | (DMSO-d6) δ 7.62-7.94 (m, 2H), 7.18-7.32 (m, 5H), 7.01 (s, 1H), 6.84- 6.90 (m, 1H), 4.15 (s, 2H), 3.82 (dt, J = 6.0, 3.1 Hz, 1H), 0.69-0.77 (m, 2H), 0.47-0.55 (m, 2H). | 392.1 |
| 149 | 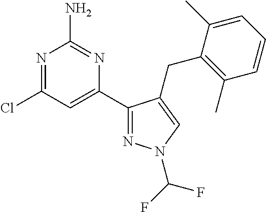 [SMILES: Cc1cccc(C)c1Cc1cn(C(F)F)nc1-c1cc(Cl)nc(N)n1] | 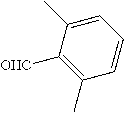 | (DMSO-d6) δ 7.53-7.86 (m, 1H), 7.28 (d, J = 3.8 Hz, 3H), 7.05-7.14 (m, 4H), 4.27 (s, 2H), 2.20 (s, 6H) | 364.1 |

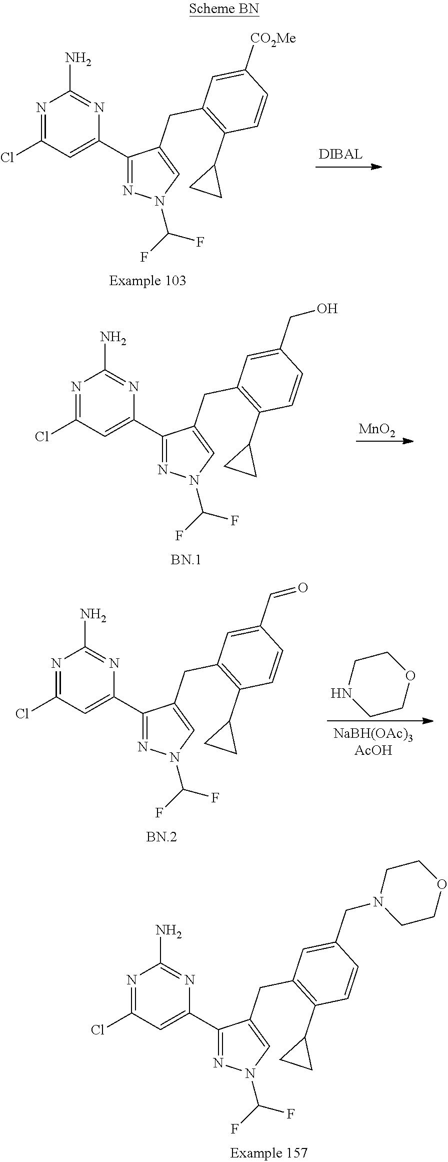

### Step 1

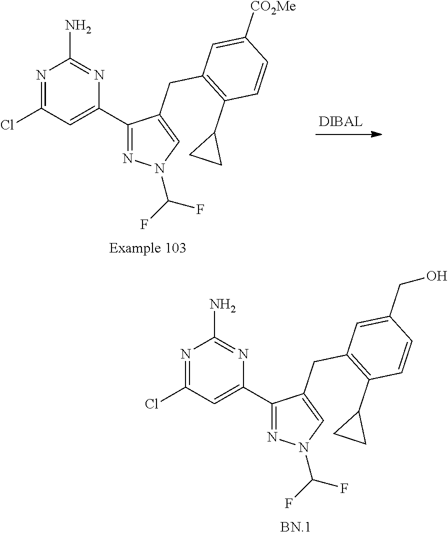 [SMILES: COC(=O)c1ccc(C2CC2)c(Cc2cn(C(F)F)nc2-c2cc(Cl)nc(N)n2)c1 ; Nc1nc(Cl)cc(-c2nn(C(F)F)cc2Cc2cc(CO)ccc2C2CC2)n1]

To a solution of methyl 3-[[3-(2-amino-6-chloro-pyrimidin-4-yl)-1-(difluoromethyl)pyrazol-4-yl]methyl]-4-cyclopropyl-benzoate (1.0 g, 2.3 mmol, 1 eq) in THF (20 mL) was added DIBAL-H (1 M, 11.5 mL, 5 eq) at −70° C. The mixture was stirred at −70° C. for 1 hr. The mixture was stirred at 20° C. for 12 hr. The reaction was quenched by addition saturated NH₄Cl solution (40 mL). The mixture was extracted with ethyl acetate (40 mL*2). The combined organic layers were dried over Na₂SO₄, filtered, and concentrated under reduced pressure. The residue was purified by column chromatography (SiO₂, Petroleum ether/Ethyl acetate=3/1 to 0/1) to furnish [3-[[3-(2-amino-6-chloro-pyrimidin-4-yl)-1-(difluoromethyl)pyrazol-4-yl]methyl]-4-cyclopropyl-phenyl]methanol.

### Step 2

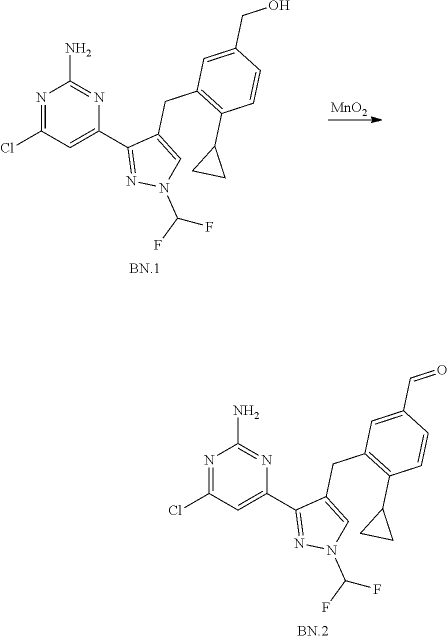 [SMILES: Nc1nc(Cl)cc(-c2nn(C(F)F)cc2Cc2cc(CO)ccc2C2CC2)n1 ; "MnO2" ; Nc1nc(Cl)cc(-c2nn(C(F)F)cc2Cc2cc(C=O)ccc2C2CC2)n1]

To a solution of [3-[[3-(2-amino-6-chloro-pyrimidin-4-yl)-1-(difluoromethyl)pyrazol-4-yl]methyl]-4-cyclopropyl-phenyl]methanol (0.200 g, 0.493 mmol, 1 eq) in DCM (15 mL) and DMF (0.8 mL) was added MnO₂ (428 mg, 4.93 mmol, 10 eq). The mixture was stirred at 20° C. for 12 hr. The reaction mixture was filtered, and the filtrate was concentrated to furnish 3-[[3-(2-amino-6-chloro-pyrimidin-4-yl)-1-(difluoromethyl)pyrazol-4-yl]methyl]-4-cyclopropyl-benzaldehyde.

### Step 3

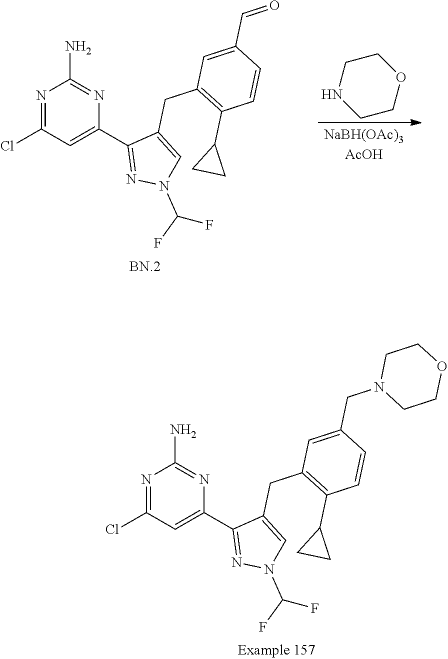 [SMILES: Nc1nc(Cl)cc(-c2nn(C(F)F)cc2Cc2cc(C=O)ccc2C2CC2)n1 ; C1COCCN1 ; Nc1nc(Cl)cc(-c2nn(C(F)F)cc2Cc2cc(CN3CCOCC3)ccc2C2CC2)n1]

To a solution of 3-[[3-(2-amino-6-chloro-pyrimidin-4-yl)-1-(difluoromethyl)pyrazol-4-yl]methyl]-4-cyclopropyl-benzaldehyde (280 mg, 0.694 mmol, 1 eq) in DCE (12 mL) and DMF (1 mL) was added AcOH (83 mg, 1.4 mmol, 0.079 mL, 2 eq) and morpholine (72 mg, 0.83 mol, 0.073 mL, 1.2 eq). After stirring at 20° C. for 0.5 hr, NaBH(OAc)₃ (294 mg, 1.39 mmol, 2 eq) was added to the reaction. The mixture was stirred at 20° C. for 11.5 hr. The reaction mixture was filtered, and the filtrate was concentrated. The residue was purified by preparativ-HPLC (column: Phenomenex Gemini-NX C18 75*30 mm*3 um; mobile phase: [water (10 mM NH₄HCO₃)-ACN]; B %: 47%-67%, 6 min) to furnish 4-chloro-6-[4-[[2-cyclopropyl-5-(morpholinomethyl)phenyl]methyl]-1-(difluoromethyl)pyrazol-3-yl]pyrimidin-2-amine Example 157.

Example 157: ¹H NMR: (DMSO-d6, 400 MHz) δ 7.62-7.95 (m, 2H), 7.24 (s, 2H), 6.99-7.07 (m, 3H), 6.89 (d, J=7.6 Hz, 1H), 4.42 (s, 2H), 3.50 (t, J=4.5 Hz, 4H), 3.33 (s, 2H), 2.24 (s, 4H), 1.85-1.93 (m, 1H), 0.77-0.84 (m, 2H), 0.52-0.59 (m, 2H); LCMS: (MH+) 475.2.

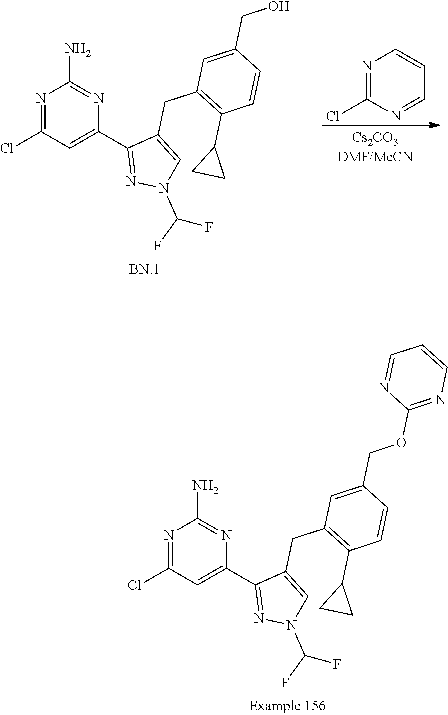 [SMILES: Nc1nc(Cl)cc(-c2nn(C(F)F)cc2Cc2cc(CO)ccc2C2CC2)n1 ; Clc1ncccn1 ; Nc1nc(Cl)cc(-c2nn(C(F)F)cc2Cc2cc(COc3ncccn3)ccc2C2CC2)n1]

To a solution of [3-[[3-(2-amino-6-chloro-pyrimidin-4-yl)-1-(difluoromethyl)pyrazol-4-yl]methyl]-4-cyclopropyl-phenyl]methanol (50 mg, 0.12 mmol, 1 eq) in DMF (1 mL) and MeCN (1 mL) was added Cs₂CO₃ (40 mg, 0.12 mmol, 1 eq) and stirred at 20° C. for 30 min under N₂. 2-Chloropyrimidine (18.34 mg, 160.17 umol, 1.3 eq) was added, and the mixture was stirred at 80° C. for 11.5 hr under N₂. The reaction mixture was filtered, and the filtrate was concentrated. The residue was purified by preparative-HPLC (column: Kromasil C18 (250*50 mm*10 um); mobile phase: [water (10 mM NH₄HCO₃)-ACN]; B %: 30%-70%, 10 min) to furnish 4-chloro-6-[4-[[2-cyclopropyl-5-(pyrimidin-2-yloxymethyl)phenyl]methyl]-1-(difluoromethyl)pyrazol-3-yl]pyrimidin-2-amine Example 156.

Example 156: ¹H NMR: (DMSO-d6, 400 MHz) δ 8.59 (d, J=4.8 Hz, 2H), 7.61-7.93 (m, 2H), 7.20-7.28 (m, 4H), 7.13 (t, J=4.8 Hz, 1H), 7.03 (s, 1H), 6.97 (d, J=7.8 Hz, 1H), 5.30 (s, 2H), 4.44 (s, 2H), 1.85-1.94 (m, 1H), 0.77-0.84 (m, 2H), 0.54-0.61 (m, 2H); LCMS: (MH+) 484.1.

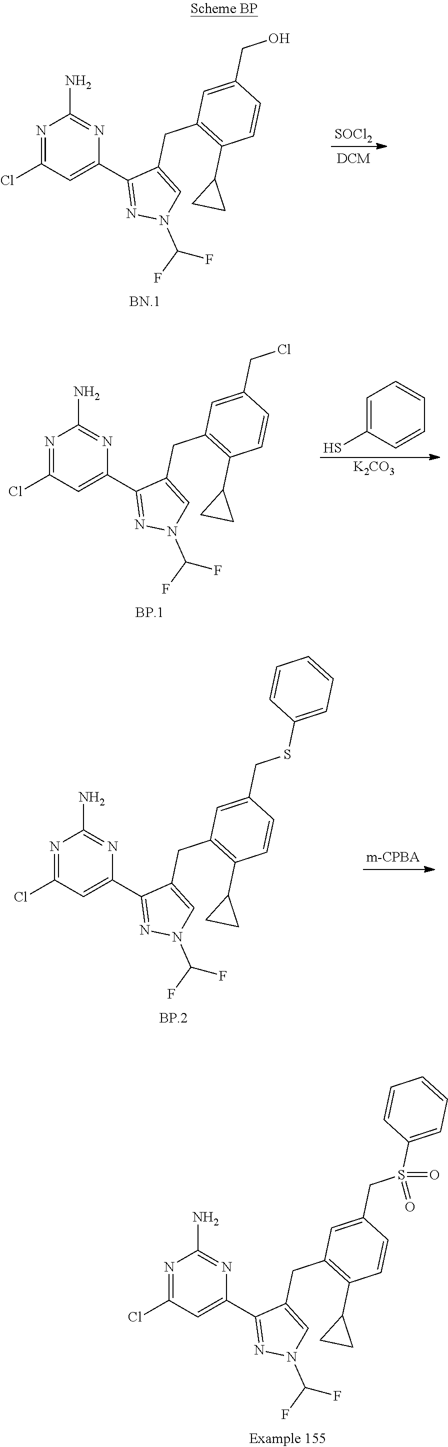

### Step 1

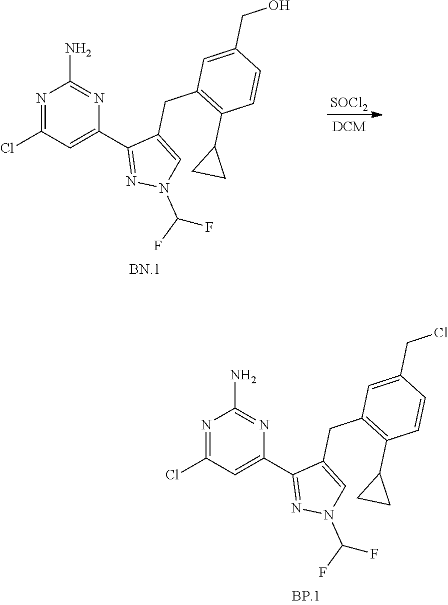

To a solution of [3-[[3-(2-amino-6-chloro-pyrimidin-4-yl)-1-(difluoromethyl)pyrazol-4-yl]methyl]-4-cyclopropyl-phenyl]methanol (120 mg, 0.30 mmol, 1 eq) in DCM (7 mL) was added SOCl₂ (493 mg, 4.14 mmol, 0.300 mL, 14 eq). The mixture was stirred at 20° C. for 4 hr. The reaction mixture was concentrated. The mixture was quenched with saturated NaHCO₃ solution (25 mL), and the mixuture was extracted with ethyl acetate (30 mL*3). The combined organic layers were dried over Na₂SO₄, filtered, and concentrated under reduced pressure. The residue was purified by column chromatography (SiO₂, Petroleum ether/Ethyl acetate=7/1 to 3/1) to furnish 4-chloro-6-[4-[[5-(chloromethyl)-2-cyclopropyl-phenyl]methyl]-1-(difluoromethyl)pyrazol-3-yl]pyrimidin-2-amine.

### Step 2

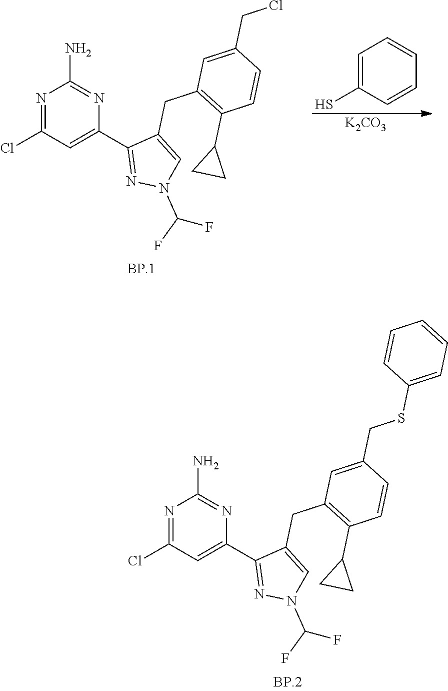

To a solution of 4-chloro-6-[4-[[5-(chloromethyl)-2-cyclopropyl-phenyl]methyl]-1-(difluoromethyl)pyrazol-3-yl]pyrimidin-2-amine (150 mg, 0.354 mmol, 1 eq) in DMF (15 mL) was added K₂CO₃ (98 mg, 0.71 mmol, 2 eq) and benzenethiol (47 mg, 0.42 mmol, 0.043 mL, 1.2 eq). The mixture was stirred at 20° C. for 4 hr. The reaction mixture was quenched by addition of H₂O (20 mL). The mixture was extracted with ethyl acetate (30 mL*3). The combined organic layers were dried over Na₂SO₄, filtered, and concentrated under reduced pressure. The residue was purified by column chromatography (SiO₂, Petroleum ether/Ethyl acetate=5/1 to 3/1) to furnish 4-chloro-6-[4-[[2-cyclopropyl-5-(phenylsulfanylmethyl)phenyl]methyl]-1-(difluoromethyl)pyrazol-3-yl]pyrimidin-2-amine.

### Step 3

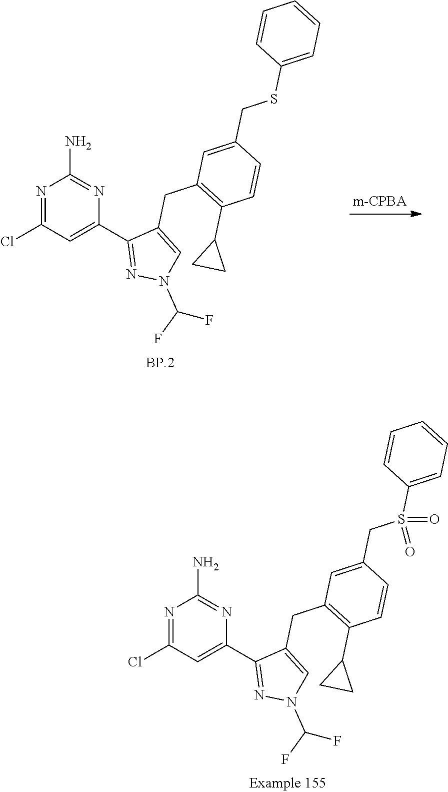 [SMILES: Nc1nc(Cl)cc(-c2nn(C(F)F)cc2Cc2cc(CSc3ccccc3)ccc2C2CC2)n1 ; Nc1nc(Cl)cc(-c2nn(C(F)F)cc2Cc2cc(CS(=O)(=O)c3ccccc3)ccc2C2CC2)n1]

To a solution of 4-chloro-6-[4-[[2-cyclopropyl-5-(phenylsulfanylmethyl)phenyl]methyl]-1-(difluoromethyl)pyrazol-3-yl]pyrimidin-2-amine (140 mg, 0.281 mmol, 1 eq) in DCM (10 mL) was added m-CPBA (91 mg, 0.42 mmol, 80% purity, 1.5 eq). The mixture was stirred at 20° C. for 6 hr. More m-CPBA (60.64 mg, 281.13 umol, 80% purity, 1 eq) was added, and the mixture was stirred at 20° C. for 12 hr. The reaction mixture was diluted with ethyl acetate (30 mL). The mixture was washed with saturated NaHSO₃ solution (20 mL), saturated NaHCO₃ solution (20 mL), and dried over Na₂SO₄. The solution was filtered, and the filtrate was concentrated under reduced pressure. The residue was purified by preparative-HPLC (column: Waters Xbridge BEH C18 100*30 mm*10 um; mobile phase: [water (10 mM NH₄HCO₃)-ACN]; B %: 40%-70%, 8 min) to furnish 4-[4-[[5-(benzenesulfonylmethyl)-2-cyclopropyl-phenyl]methyl]-1-(difluoromethyl)pyrazol-3-yl]-6-chloro-pyrimidin-2-amine Example 155.

Example 155: ¹H NMR: (DMSO-d6, 400 MHz) δ 7.61-7.97 (m, 4H), 7.48-7.55 (m, 2H), 7.44 (s, 1H), 7.23 (s, 2H), 7.04 (s, 1H), 6.96 (d, J=7.9 Hz, 1H), 6.84-6.90 (m, 2H), 4.56 (s, 2H), 4.32 (s, 2H), 1.85 (s, 1H), 0.79 (d, J=8.4 Hz, 2H), 0.57 (s, 2H); LCMS: (MH+) 530.1.

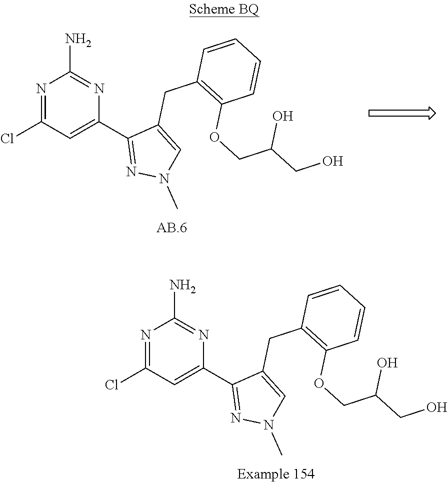 [SMILES: Cn1cc(Cc2ccccc2OCC(O)CO)c(-c2cc(Cl)nc(N)n2)n1 ; Cn1cc(Cc2ccccc2OCC(O)CO)c(-c2cc(Cl)nc(N)n2)n1]

Example 154: ¹H NMR: (DMSO-d6, 400 MHz) δ 7.53 (s, 1H), 7.18 (dd, J=1.6, 7.3 Hz, 1H), 7.13-7.11 (m, 1H), 7.06 (s, 2H), 6.98 (s, 1H), 6.92 (d, J=7.7 Hz, 1H), 6.77-6.79 (m, 1H), 4.99 (d, J=5.0 Hz, 1H), 4.66 (t, J=5.7 Hz, 1H), 4.25 (s, 2H), 4.04-3.96 (m, 1H), 3.92-3.87 (m, 1H), 3.85-3.81 (m, 1H), 3.81 (s, 3H), 3.48-3.42 (m, 2H); LCMS: (MH+) 390.0.

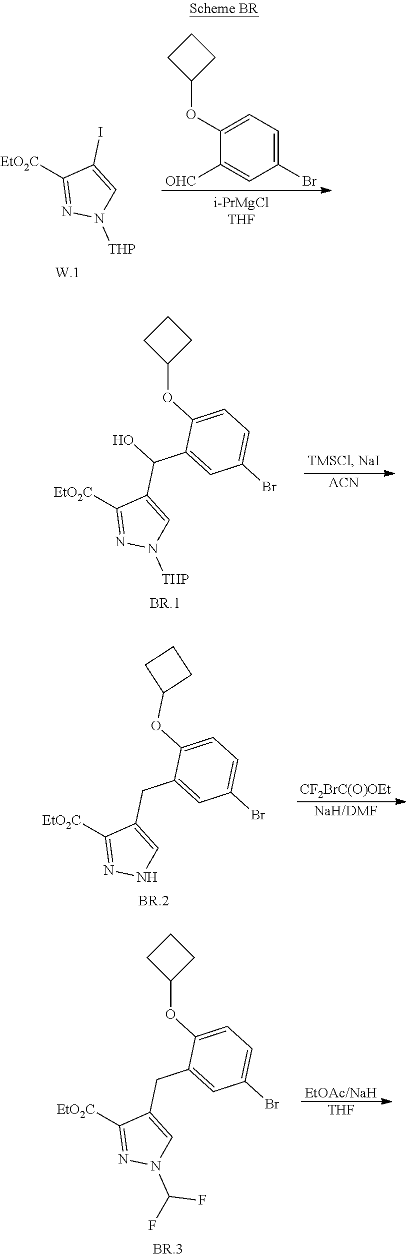

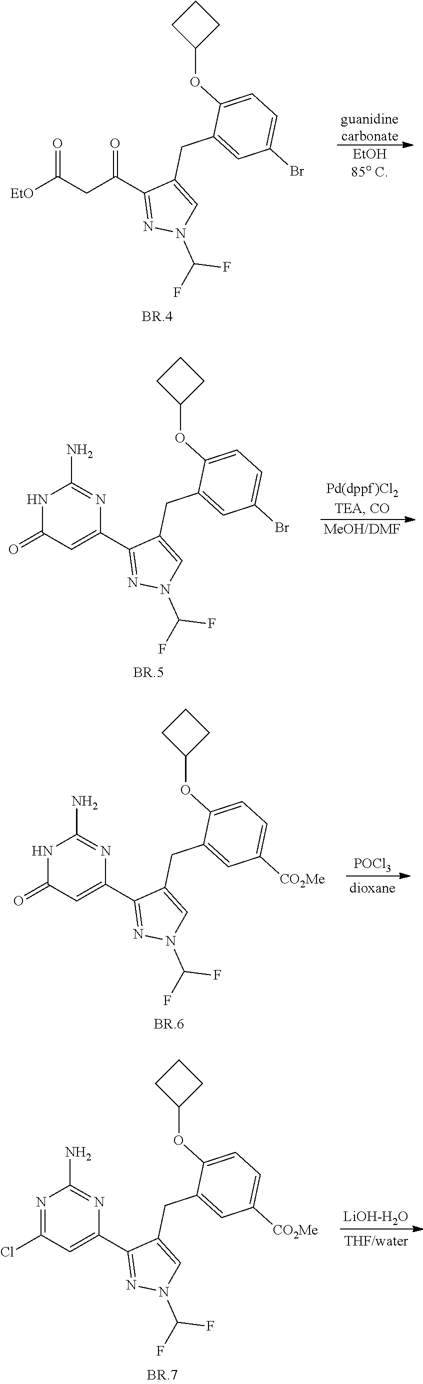

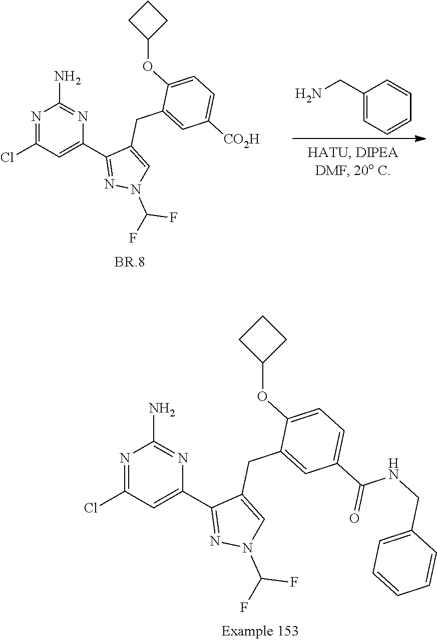 [SMILES: Nc1nc(Cl)cc(-c2nn(C(F)F)cc2Cc2cc(C(=O)O)ccc2OC2CCC2)n1 ; NCc1ccccc1 ; Nc1nc(Cl)cc(-c2nn(C(F)F)cc2Cc2cc(C(=O)NCc3ccccc3)ccc2OC2CCC2)n1]

Example 153 was prepared in a similar fashion to that described in Scheme AI using the appropriate aldehyde in Step 1.

Example 153: ¹H NMR: (DMSO-d6, 400 MHz) δ 8.83-8.76 (m, 1H), 7.96-7.64 (m, 4H), 7.34-7.18 (m, 7H), 7.04 (s, 1H), 6.86 (d, J=8.8 Hz, 1H), 4.79-4.70 (m, 1H), 4.44 (d, J=5.7 Hz, 2H), 4.21 (s, 2H), 2.38 (brs, 2H), 1.97-1.83 (m, 2H), 1.79-1.55 (m, 2H); LCMS: (MH+) 539.0.

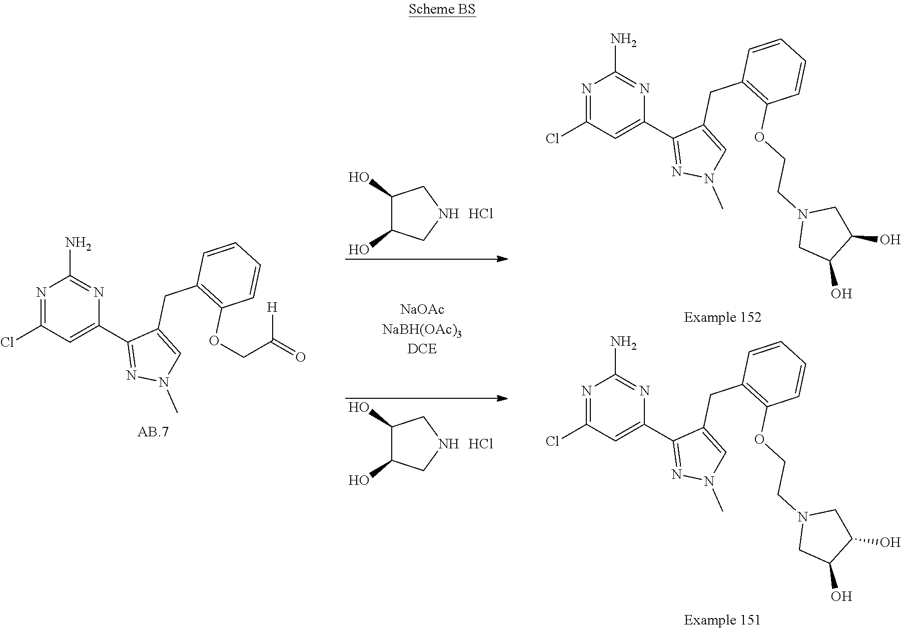

Example 151 and Example 152 were prepared using similar conditions outlined in Scheme AB using the appropriates amines in the last step (Scheme BS).

Example 152: ¹H NMR: (MeOH-d4, 400 MHz) δ 7.19-7.12 (m, 3H), 7.10 (s, 1H), 6.93 (d, J=7.9 Hz, 1H), 6.95-6.91 (m, 1H), 6.86 (t, J=7.5 Hz, 1H), 4.26 (s, 2H), 4.11-4.02 (m, 4H), 3.85 (s, 3H), 2.91 (dd, J=6.1, 10.5 Hz, 2H), 2.84 (t, J=5.4 Hz, 2H), 2.61 (dd, J=4.2, 10.3 Hz, 2H); LCMS: (MH+) 445.2.

Example 151: ¹H NMR: (MeOH-d4, 400 MHz) δ 7.20-7.12 (m, 3H), 7.10 (s, 1H), 6.95-6.83 (m, 2H), 4.26 (s, 2H), 4.09 (t, J=5.4 Hz, 2H), 3.99 (t, J=3.8 Hz, 2H), 3.85 (s, 3H), 3.02 (br dd, J=5.3, 10.4 Hz, 2H), 2.96-2.80 (m, 2H), 2.60 (br d, J=8.5 Hz, 2H); LCMS: (MH+) 445.2.

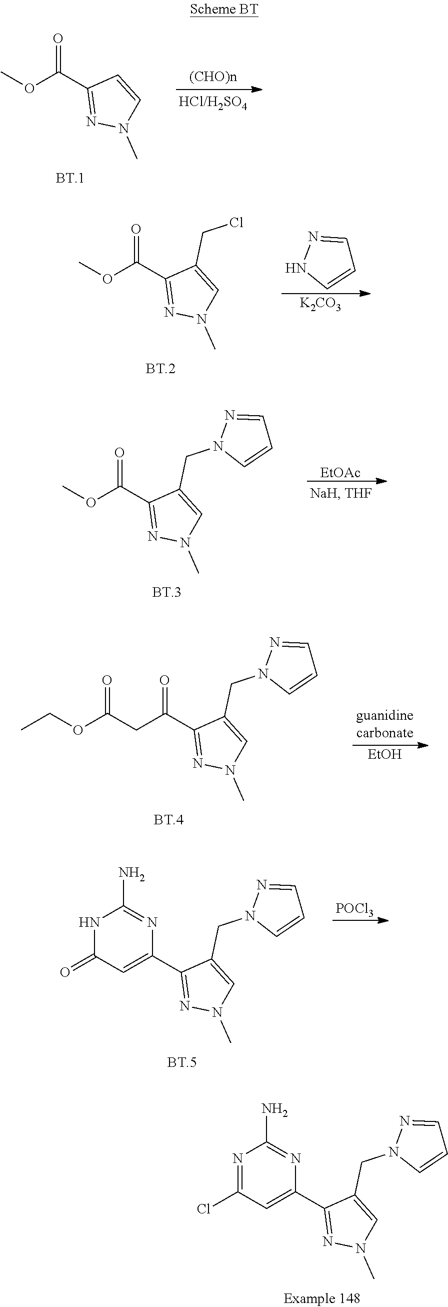

### Step 1

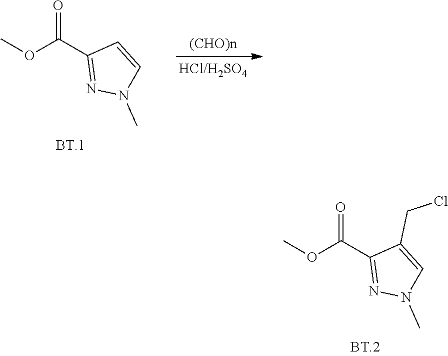

To a solution of methyl 1-methylpyrazole-3-carboxylate (5.00 g, 35.7 mmol, 1 eq) and (CHO)n (7.29 g, 143 mmol, 4 eq) in dioxane (100 mL) was added HCl (12 M, 11.89 mL) and H₂SO₄ (0.389 mL, 98% purity). The mixture was stirred at 100° C. for 5 hr. The reaction mixture was concentrated under reduced pressure. The residue was quenched by addition saturated NaHCO₃ solution (100 ml). The mixture was extracted with ethyl acetate (100 mL*3). The combined organic layers were dried over Na₂SO₄, filtered, and concentrated. The residue was purified by column chromatography (SiO₂, Petroleum ether/Ethyl acetate=3/1 to 13/7). To furnish methyl 4-(chloromethyl)-1-methyl-pyrazole-3-carboxylate.

### Step 2

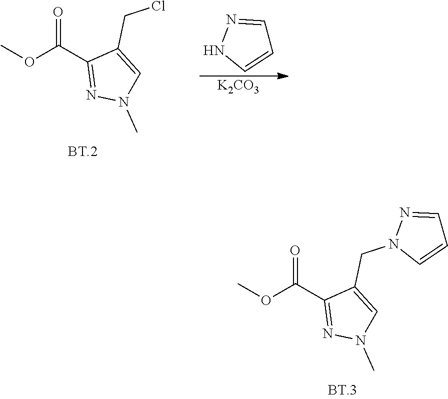

To a solution of methyl 4-(chloromethyl)-1-methyl-pyrazole-3-carboxylate (3.00 g, 15.9 mmol, 1 eq) and 1H-pyrazole (1.19 g, 17.5 mmol, 1.1 eq) in DMF (60 mL) was added K₂CO₃ (5.50 g, 39.8 mmol, 2.5 eq). The mixture was stirred at 60° C. for 12 hr. The reaction mixture was filtered and concentrated under reduced pressure. The residue was purified by column chromatography (SiO₂, Petroleum ether/Ethyl acetate=1/3 to 0/1) to furnish methyl 1-methyl-4-(pyrazol-1-ylmethyl)pyrazole-3-carboxylate.

### Step 3

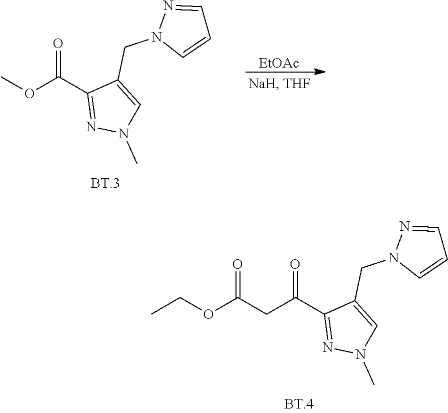 [SMILES: "EtOAc" ; COC(=O)c1nn(C)cc1Cn1cccn1 ; CCOC(=O)CC(=O)c1nn(C)cc1Cn1cccn1]

To a solution of EtOAc (1.40 g, 15.9 mmol, 1.56 mL, 7 eq) in THF (10 mL) was added NaH (272 mg, 6.81 mmol, 60 wt % in oil, 3 eq). The mixture was stirred at 20° C. for 30 min. To the mixture was added methyl 1-methyl-4-(pyrazol-1-ylmethyl)pyrazole-3-carboxylate (500 mg, 2.27 mmol, 1 eq). The mixture was stirred at 20° C. for 12 hr. The reaction was quenched by addition H₂O (20 ml), and the mixture was extracted with ethyl acetate (20 mL*3). The combined organic layers were dried over Na₂SO₄, filtered, and concentrated under reduced pressure. The residue was purified by column chromatography (SiO₂, Petroleum ether/Ethyl acetate=1/3 to 1/4) to furnish ethyl 3-[1-methyl-4-(pyrazol-1-ylmethyl)pyrazol-3-yl]-3-oxo-propanoate.

### Step 4

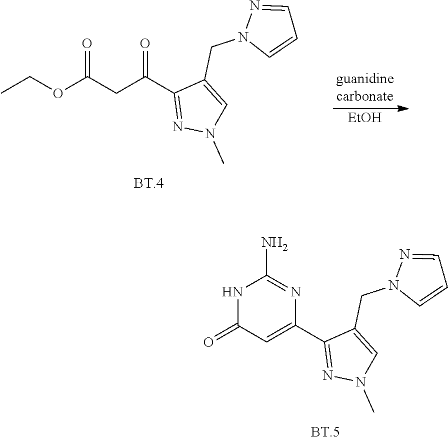 [SMILES: "EtOH" ; CCOC(=O)CC(=O)c1nn(C)cc1Cn1cccn1 ; Cn1cc(Cn2cccn2)c(-c2cc(=O)[nH]c(N)n2)n1]

To a solution of ethyl 3-[1-methyl-4-(pyrazol-1-ylmethyl)pyrazol-3-yl]-3-oxo-propanoate (300 mg, 1.09 mmol, 1 eq) in EtOH (6 mL) was added guanidine carbonate (587 mg, 3.26 mmol, 3 eq) under N₂. The mixture was stirred at 80° C. for 12 hr. The reaction mixture was filtered and concentrated under reduced pressure. The residue was triturated with methyl tert-butyl ether at 20° C. for 30 min to furnish 2-amino-4-[1-methyl-4-(pyrazol-1-ylmethyl)pyrazol-3-yl]-1H-pyrimidin-6-one.

### Step 5

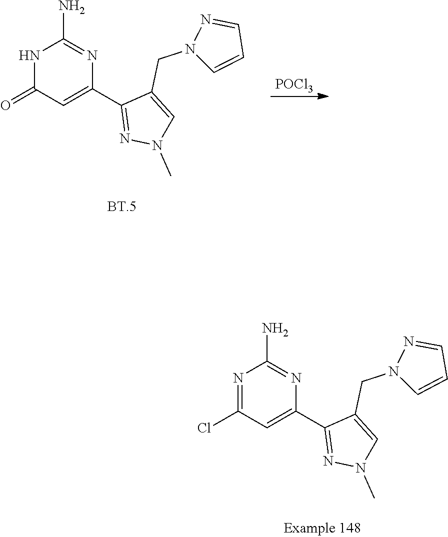

To a solution of 2-amino-4-[1-methyl-4-(pyrazol-1-ylmethyl)pyrazol-3-yl]-1H-pyrimidin-6-one (60 mg, 0.22 mmol, 1 eq) in CH₃CN (2 mL) was added POCl₃ (509 mg, 3.32 mmol, 0.31 mL, 15 eq) and TEA (45 mg, 0.44 mmol, 0.061 mL, 2 eq). The mixture was stirred at 75° C. for 4 hr. The reaction mixture was concentrated under reduced pressure. The reaction was quenched by addition of a saturated NaHCO₃ solution (10 ml). The mixture was extracted with ethyl acetate (10 mL*3). The combined organic layers were dried over Na₂SO₄, filtered, and concentrated under reduced pressure. The residue was purified by preparative-HPLC (column: Phenomenex Gemini-NX C18 75*30 mm*3 um; mobile phase: [water (10 Mm NH₄HCO₃)-ACN]; B %: 15%-40%, 8 min) to furnish 4-chloro-6-[1-methyl-4-(pyrazol-1-ylmethyl)pyrazol-3-yl]pyrimidin-2-amine Example 148.

Example 148: ¹H NMR: (DMSO-d6, 400 MHz) δ 7.84 (s, 1H), 7.54 (s, 1H), 7.41 (s, 1H), 7.20 (s, 2H), 7.01 (s, 1H), 6.19 (s, 1H), 5.64 (s, 2H), 3.85 (s, 3H); LCMS: (MH+) 290.1.

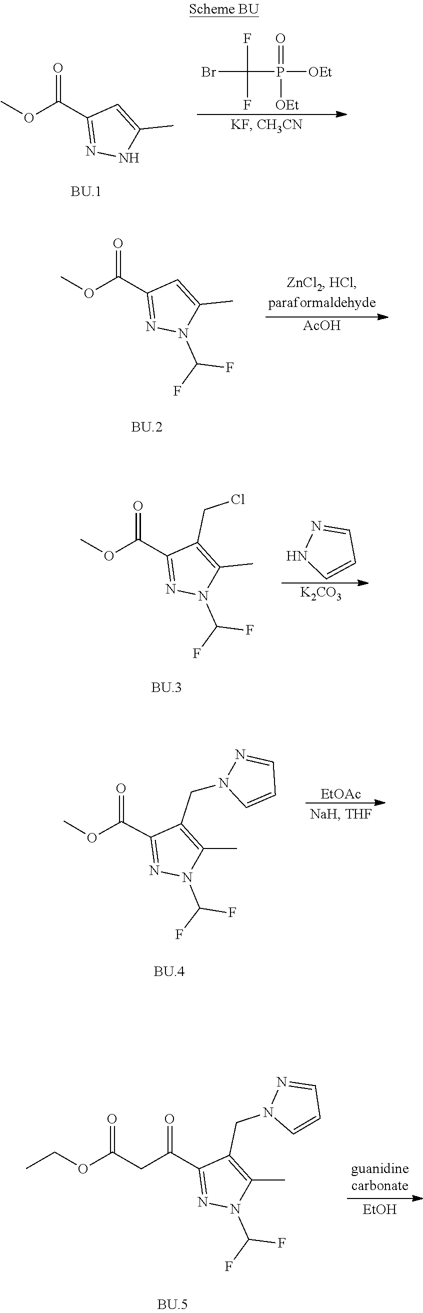

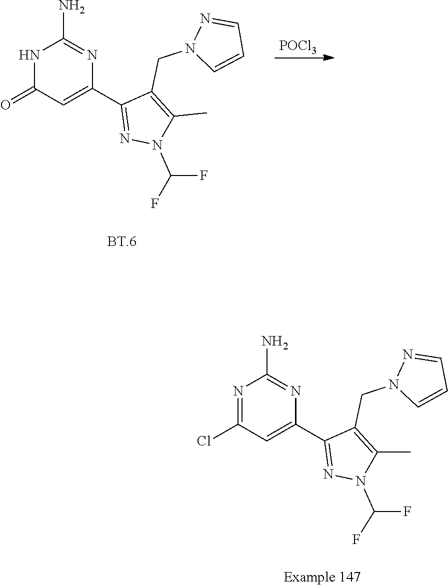

### Step 1

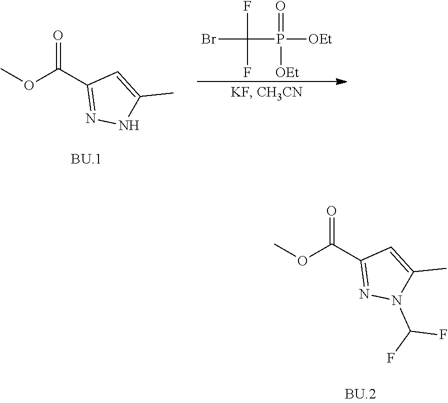

To a solution of ethyl 5-methyl-1H-pyrazole-3-carboxylate (2.70 g, 17.5 mmol, 1 eq) in MeCN (40 mL) was added KF (2.04 g, 35.0 mmol, 2 eq) and 1-[[bromo(difluoro)methyl]-ethoxy-phosphoryl]oxyethane (9.35 g, 35.0 mmol, 2 eq). The mixture was stirred at 20° C. for 12 hr. The reaction mixture was quenched with H₂O (50 mL). The mixture was extracted with ethyl acetate (60 mL*3). The combined organic layers were dried over Na₂SO₄, filtered, and concentrated under reduced pressure. The residue was purified by column chromatography (SiO₂, Petroleum ether/Ethyl acetate=20/1 to 13/1) to furnish 1-(difluoromethyl)-5-methyl-pyrazole-3-carboxylate.

### Step 2

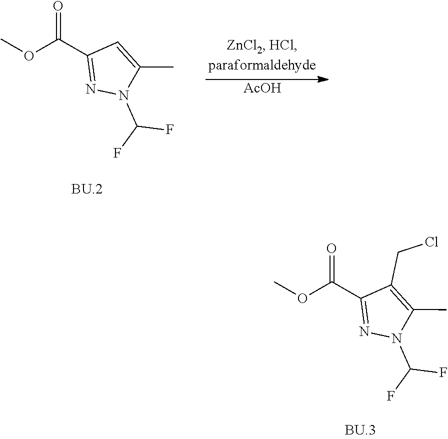

To a solution of ethyl 1-(difluoromethyl)-5-methyl-pyrazole-3-carboxylate (1.80 g, 8.82 mmol, 1 eq) and paraformaldehyde (0.81 g, 26.5 mmol, 3 eq) in AcOH (30 mL) was added ZnCl₂ (3.60 g, 26.5 mmol, 1.24 mL, 3 eq) and HCl (12 M, 2.20 mL, 3 eq). The mixture was stirred at 60° C. for 12 hr. The reaction was quenched with H₂O (30 mL). The mixture was extracted with ethyl acetate (40 mL*3). The combined organic layers were dried over Na₂SO₄, filtered, and concentrated under reduced pressure. The residue was purified by column chromatography (SiO₂, Petroleum ether/Ethyl acetate=10/1 to 3/1) to furnish ethyl 4-(chloromethyl)-1-(difluoromethyl)-5-methyl-pyrazole-3-carboxylate.

Intermediate BU.3 was converted into Example 147 using conditions like those outlined in Scheme BT.

Example 147: ¹H NMR: (methanol-d4, 400 MHz) δ 7.41-7.73 (m, 3H), 7.21 (s, 1H), 6.21 (t, J=2.1 Hz, 1H), 5.74 (s, 2H), 2.50 (s, 3H); LCMS: (MH+) 340.1.

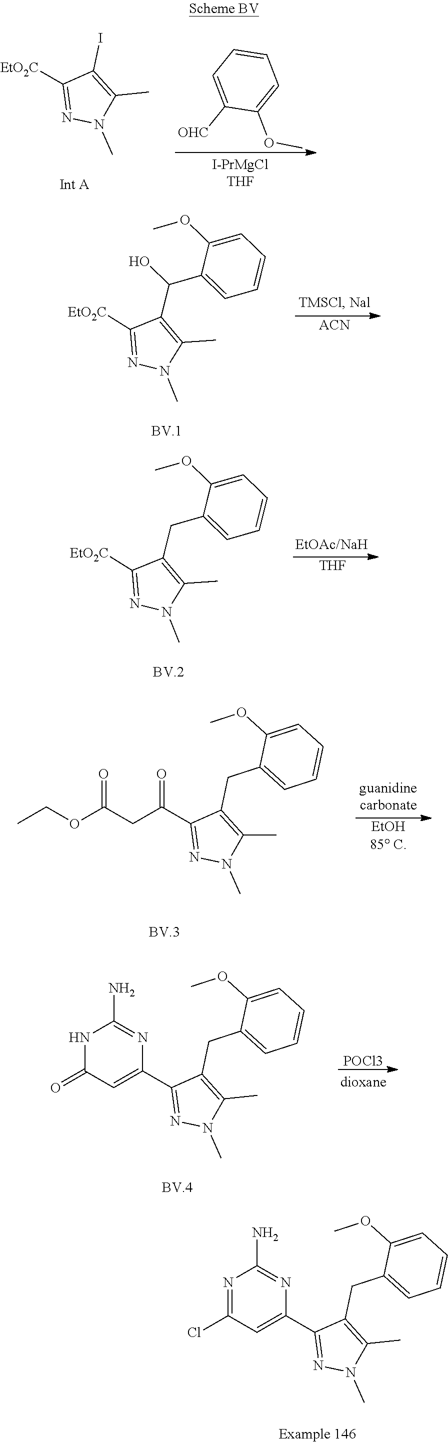

Example 146 was prepared from Int A using conditions like those outlined in Scheme B using the appropriate aldehyde in Step 1 of Scheme BV.

Example 146: ¹H NMR: (DMSO-d6, 400 MHz) δ 7.13-7.06 (m, 1H), 6.96-6.88 (m, 3H), 6.76-6.70 (m, 1H), 4.22 (s, 2H), 3.77 (d, J=6.5 Hz, 6H), 2.11 (s, 3H); LCMS: (MH+) 344.1.

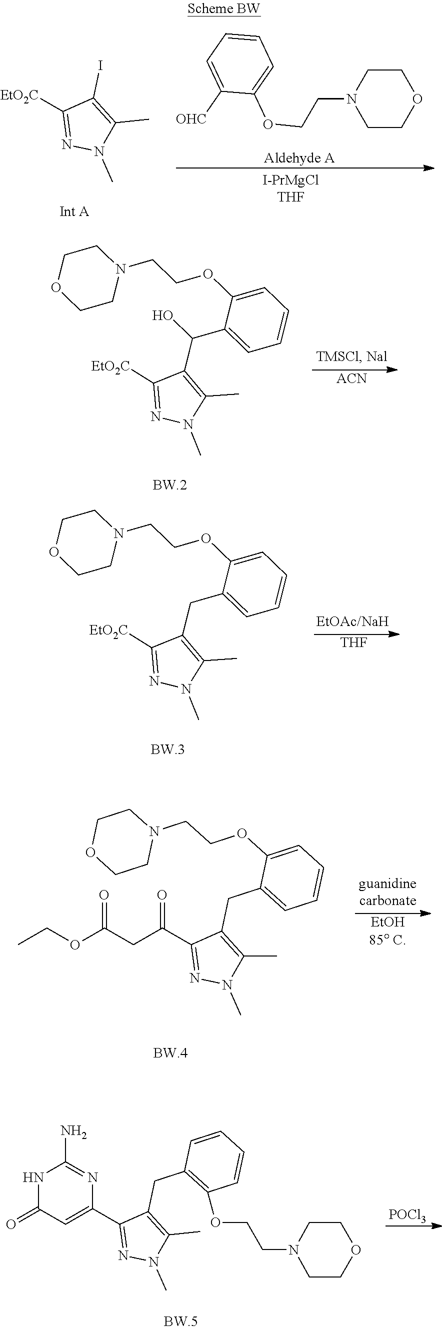

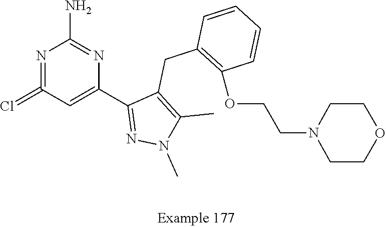

Example 177 was prepared using conditions like those outlined in Scheme B using Aldehyde A in Step 1 (Scheme BW).

Example 177: ¹H NMR: (DMSO-d6, 400 MHz) δ 7.11-7.05 (m, 1H), 7.00-6.89 (m, 5H), 6.79-6.71 (m, 1H), 4.26 (s, 2H), 4.06 (t, J=5.9 Hz, 2H), 3.77 (s, 3H), 3.60-3.54 (m, 4H), 2.63 (t, J=5.9 Hz, 2H), 2.47 (br d, J=4.1 Hz, 4H), 2.08 (s, 3H); LCMS: (MH+) 443.2.

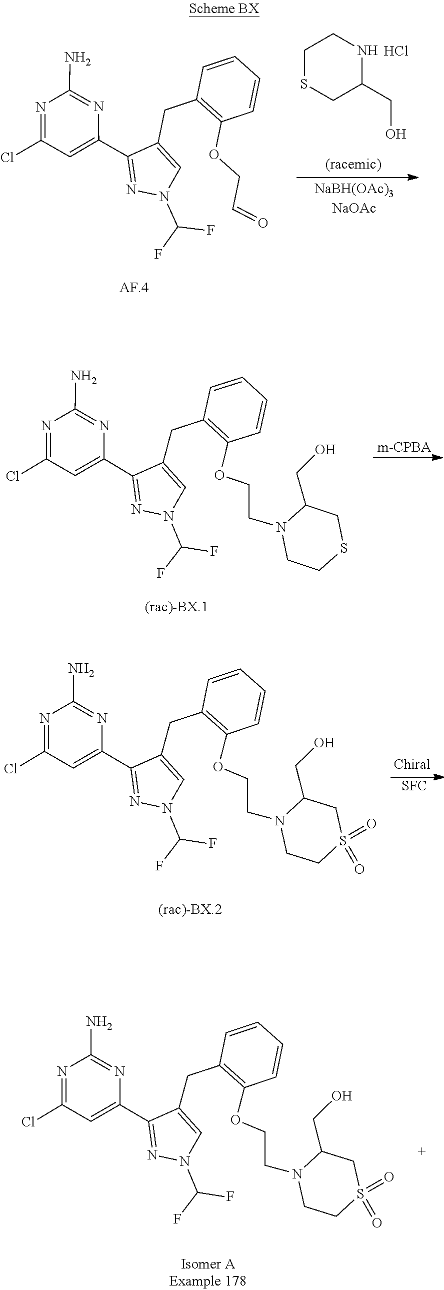

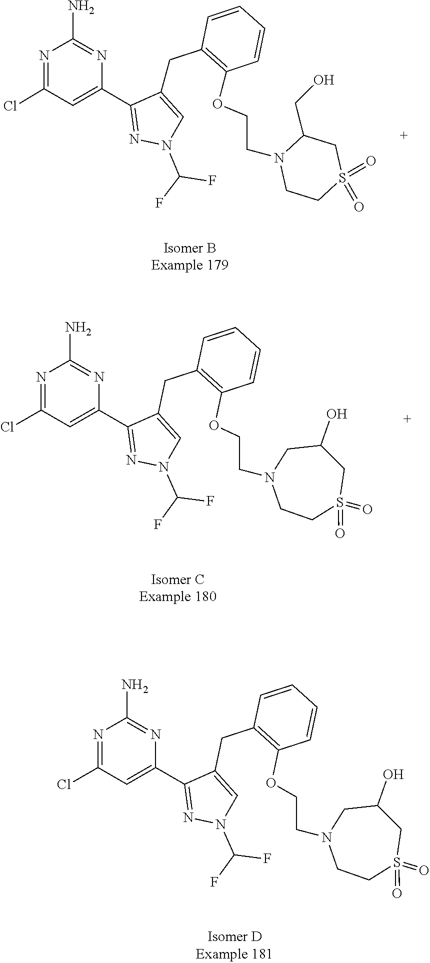

### Step 1

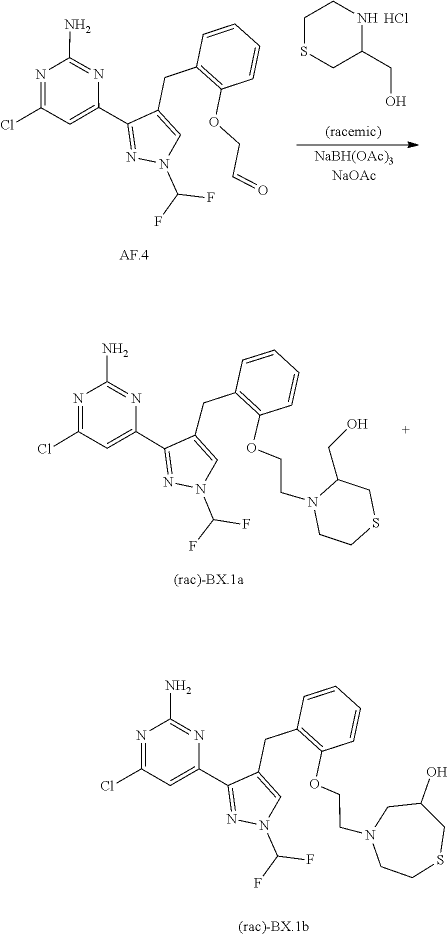

To a solution of 3-[2-[[3-(2-amino-6-chloro-pyrimidin-4-yl)-1-(difluoromethyl)pyrazol-4-yl]methyl]phenoxy]propane-1,2-diol (1.14 g, 2.68 mmol, 1 eq) in dioxane (28 mL) and H₂O (8.40 mL) was added NaIO₄ (1.43 g, 6.69 mmol, 0.371 mL, 2.5 eq). The mixture was stirred at 20° C. for 1 hr. The reaction mixture was quenched by addition of saturated Na₂SO₃ solution (80 mL). The mixture was extracted with ethyl acetate (80 mL*3). The combined organic layers were dried over Na₂SO₄, filtered, and concentrated under reduced pressure to furnish 2-[2-[[3-(2-amino-6-chloro-pyrimidin-4-yl)-1-(difluoromethyl)pyrazol-4-yl]methyl]phenoxy] acetaldehyde.

### Step 2

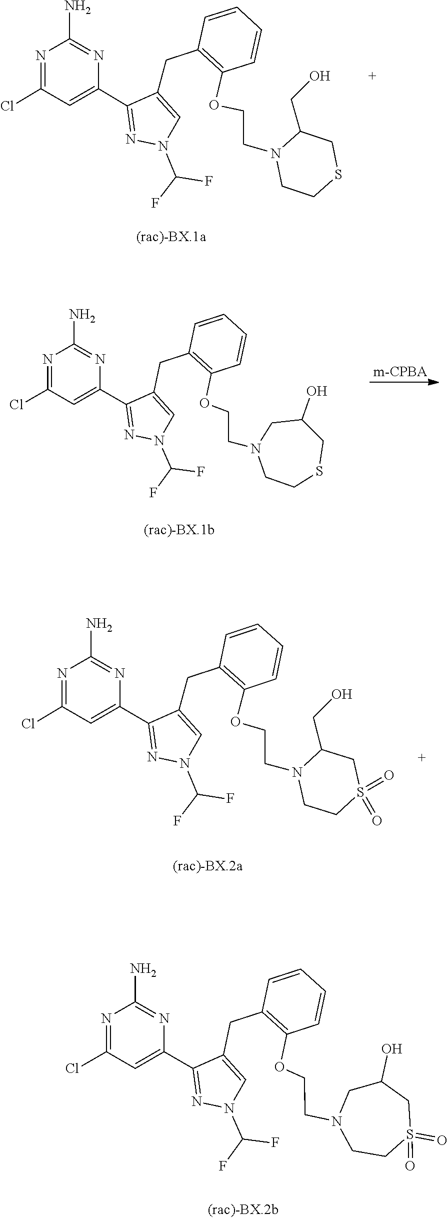

To a solution of [4-[2-[2-[[3-(2-amino-6-chloro-pyrimidin-4-yl)-1-(difluoromethyl)pyrazol-4-yl]methyl]phenoxy]ethyl]thiomorpholin-3-yl]methanol (260 mg, 0.509 mmol, 1 eq) in DCM (30 mL) was added m-CPBA (83 mg, 0.41 mmol, 85% purity, 0.8 eq). The mixture was stirred at 20° C. for 12 hr. Additional m-CPBA (52 mg, 0.25 mmol, 85% purity, 0.5 eq) was added, and the reaction was stirred for 12 hr at 20° C. More m-CPBA (41 mg, 0.20 mmol, 85% purity, 0.4 eq) was added, and the reaction was stirred for 3 hr at 20° C. Additional m-CPBA (10 mg, 0.051 mmol, 85% purity, 0.1 eq) was added, and the reaction was stirred for 5 hr at 20° C. The reaction mixture was diluted with ethyl acetate (120 mL) and washed with saturated Na₂SO₃ solution (60 mL), saturated NaHCO₃ solution (60 mL), and dried over Na₂SO₄. The mixture was filtered, and the filtrate was concentrated under reduced pressure. The residue was purified by prep-HPLC (column: Waters Xbridge BEH C18 100*30 mm*10 um; mobile phase: [water (10 mM NH₄HCO₃)-ACN]; B %: 15%-45%, 8 min).

### Step 3

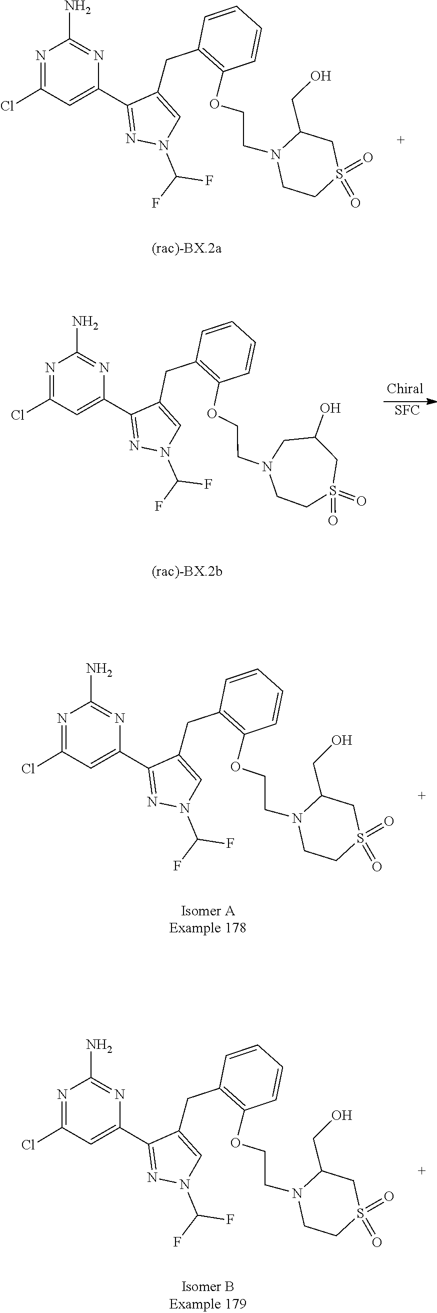

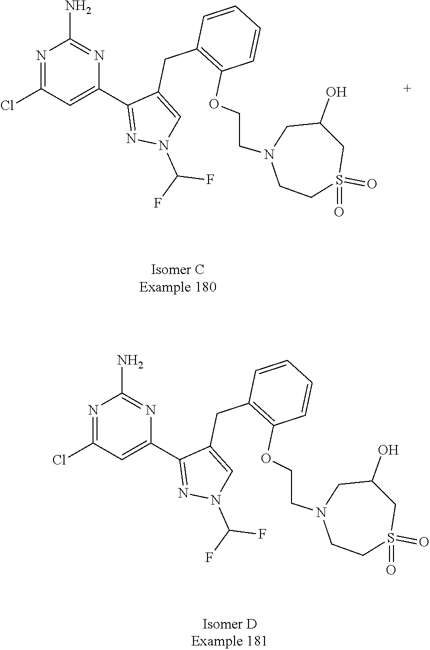 [SMILES: Nc1nc(Cl)cc(-c2nn(C(F)F)cc2Cc2ccccc2OCCN2CCS(=O)(=O)CC(O)C2)n1 ; Nc1nc(Cl)cc(-c2nn(C(F)F)cc2Cc2ccccc2OCCN2CCS(=O)(=O)CC(O)C2)n1]

The mixture of 4 isomers was separated by SFC (column: DAICEL CHIRALPAK AD (250 mm*30 mm, 10 um); mobile phase: [0.1% NH₃H₂O IPA]; B %: 44%-44%, 9 min) to furnish (in order of elution) Isomer A Example 178, Isomer B Example 179, Isomer C Example 180, and Isomer D Example 181.

Example 178: ¹H NMR: (chloroform-D, 400 MHz) δ 7.32-7.28 (m, 1H), 7.25 (d, J=8.3 Hz, 2H), 7.17-7.12 (m, 2H), 7.03-6.97 (m, 3H), 6.93 (d, J=8.3 Hz, 1H), 5.71-5.50 (m, 2H), 4.78-4.64 (m, 1H), 4.60-4.50 (m, 1H), 4.41-4.31 (m, 1H), 4.29-4.05 (m, 6H), 4.02-3.88 (m, 2H), 3.64-3.44 (m, 2H), 3.37-3.25 (m, 1H), 2.91-2.80 (m, 1H), 2.75-2.62 (m, 1H); LCMS: (MH+) 543.2.

Example 179: ¹H NMR: (chloroform-D, 400 MHz) δ 7.33-7.29 (m, 1H), 7.24 (d, J=12.3 Hz, 2H), 7.15 (d, J=6.1 Hz, 2H), 7.04-6.98 (m, 1H), 6.94 (d, J=8.3 Hz, 1H), 5.67-5.51 (m, 2H), 4.78-4.65 (m, 1H), 4.55-4.45 (m, 1H), 4.43-4.33 (m, 1H), 4.31-4.02 (m, 6H), 3.94-3.81 (m, 1H), 3.61-3.48 (m, 2H), 3.46-3.36 (m, 1H), 2.86 (dd, J=1.2, 14.8 Hz, 1H), 2.75-2.65 (m, 1H); LCMS: (MH+) 543.2.

Example 180: ¹H NMR: (chloroform-D, 400 MHz) δ 7.38-7.28 (m, 1H), 7.27-7.14 (m, 3H), 7.10-7.04 (m, 1H), 7.02-6.92 (m, 2H), 5.86-5.60 (m, 2H), 4.87-4.75 (m, 1H), 4.74-4.58 (m, 2H), 4.45-3.97 (m, 6H), 3.84-3.49 (m, 4H), 2.97-2.76 (m, 2H); LCMS: (MH+) 543.2.

Example 181: ¹H NMR: (chloroform-D, 400 MHz) δ 7.40 (s, 1H), 7.25 (s, 1H), 7.21-7.14 (m, 2H), 7.06-6.90 (m, 3H), 5.94-5.59 (m, 2H), 4.88-4.77 (m, 1H), 4.73-4.59 (m, 2H), 4.46-3.99 (m, 6H), 3.78-3.46 (m, 4H), 2.94-2.78 (m, 2H); LCMS: (MH+) 543.2.

### Compounds and sAc Inhibitory Activity

### sAC Biochemical Cyclase Assay

Assays for sAC activity using purified protein were performed in 100 μl reactions containing 4 mM MgCl₂, 2 mM CaCl₂, 1 mM ATP, 40 mM NaHCO₃, 50 mM Tris pH 7.5, and 3 mM DTT. Each reaction contained ˜1,000,000 counts of α-³²P labeled ATP. Generated cAMP was purified using sequential Dowex and Alumina chromatography as previously described (Salomon et al., (1979) Adenylate cyclase assay. Adv Cyclic Nucleotide Res 10:35-55). Data for representative examples is shown in Table A.

TABLE A
sAC Biochemical Cyclase Assay

| | | SAC IC₅₀ |
|---|---|---|
| Example No. | Structure | (nM) |
| 1 | 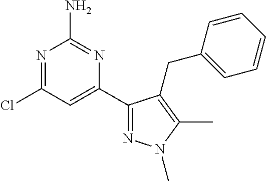 [SMILES: Cc1c(Cc2ccccc2)c(-c2cc(Cl)nc(N)n2)nn1C] | 159 |
| 2 | 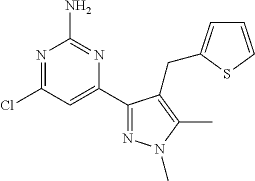 [SMILES: Cc1c(Cc2cccs2)c(-c2cc(Cl)nc(N)n2)nn1C] | 50 |
| 3 | 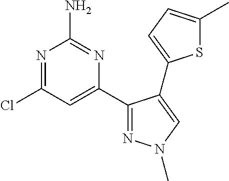 [SMILES: Cc1ccc(-c2cn(C)nc2-c2cc(Cl)nc(N)n2)s1] | 1000-10000 |
| 4 | 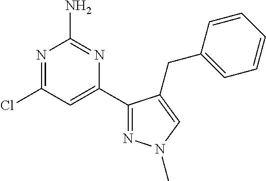 [SMILES: Cn1cc(Cc2ccccc2)c(-c2cc(Cl)nc(N)n2)n1] | 550 |
| 5 | 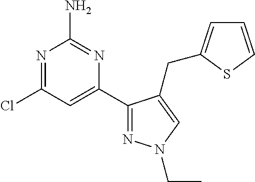 [SMILES: CCn1cc(Cc2cccs2)c(-c2cc(Cl)nc(N)n2)n1] | 180 |
| 6 | 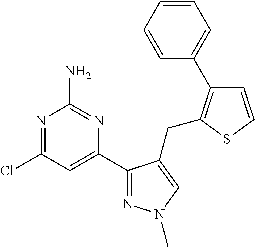 [SMILES: Cn1cc(Cc2sccc2-c2ccccc2)c(-c2cc(Cl)nc(N)n2)n1] | 220 |
| 7 | 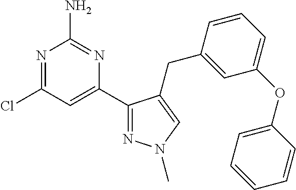 [SMILES: Cn1cc(Cc2cccc(Oc3ccccc3)c2)c(-c2cc(Cl)nc(N)n2)n1] | 220 |
| 8 | 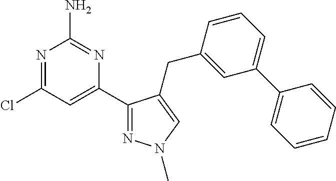 [SMILES: Cn1cc(Cc2cccc(-c3ccccc3)c2)c(-c2cc(Cl)nc(N)n2)n1] | 140 |
| 9 | 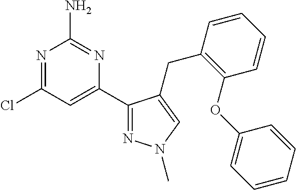 [SMILES: Cn1cc(Cc2ccccc2Oc2ccccc2)c(-c2cc(Cl)nc(N)n2)n1] | 100 |
| 10 | 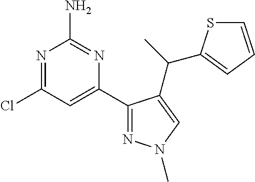 [SMILES: CC(c1cccs1)c1cn(C)nc1-c1cc(Cl)nc(N)n1] | 105 |
| 11 | 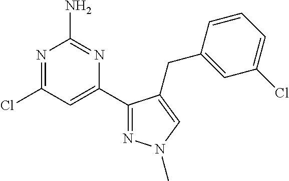 [SMILES: Cn1cc(Cc2cccc(Cl)c2)c(-c2cc(Cl)nc(N)n2)n1] | 75 |
| 12 | 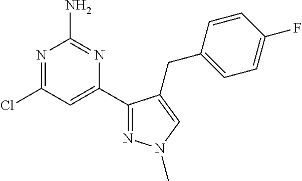 [SMILES: Cn1cc(Cc2ccc(F)cc2)c(-c2cc(Cl)nc(N)n2)n1] | ~1000 |
| 13 | 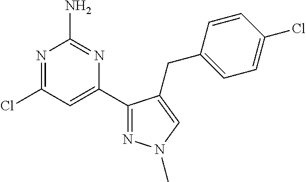 [SMILES: Cn1cc(Cc2ccc(Cl)cc2)c(-c2cc(Cl)nc(N)n2)n1] | ~1000 |
| 14 | 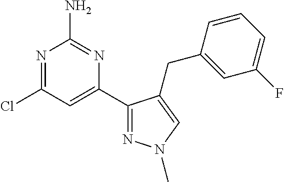 [SMILES: Cn1cc(Cc2cccc(F)c2)c(-c2cc(Cl)nc(N)n2)n1] | 100 |
| 15 | 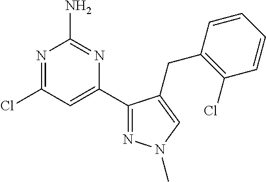 [SMILES: Cn1cc(Cc2ccccc2Cl)c(-c2cc(Cl)nc(N)n2)n1] | 60 |
| 16 | 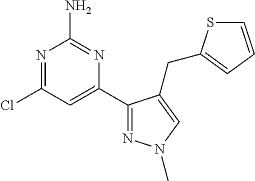 [SMILES: Cn1cc(Cc2cccs2)c(-c2cc(Cl)nc(N)n2)n1] | 141 |
| 17 | 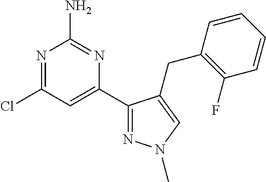 [SMILES: Cn1cc(Cc2ccccc2F)c(-c2cc(Cl)nc(N)n2)n1] | 120 |
| 18 | 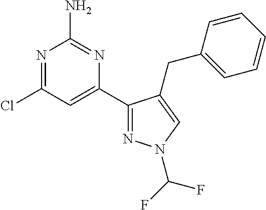 [SMILES: Nc1nc(Cl)cc(-c2nn(C(F)F)cc2Cc2ccccc2)n1] | 110 |
| 19 | 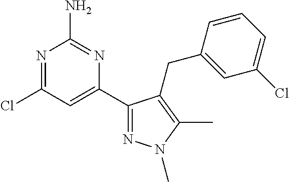 [SMILES: Cc1c(Cc2cccc(Cl)c2)c(-c2cc(Cl)nc(N)n2)nn1C] | 130 |
| 20 | 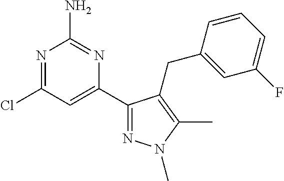 [SMILES: Cc1c(Cc2cccc(F)c2)c(-c2cc(Cl)nc(N)n2)nn1C] | 160 |
| 21 | 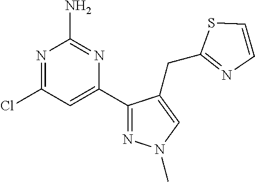 [SMILES: Cn1cc(Cc2nccs2)c(-c2cc(Cl)nc(N)n2)n1] | 260 |
| 22 | 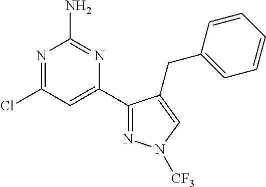 [SMILES: Nc1nc(Cl)cc(-c2nn(C(F)(F)F)cc2Cc2ccccc2)n1] | 160 |
| 23 | 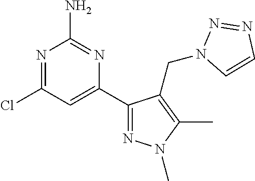 [SMILES: Cc1c(Cn2ccnn2)c(-c2cc(Cl)nc(N)n2)nn1C] | 700 |
| 24 | 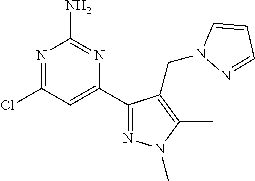 [SMILES: Cc1c(Cn2cccn2)c(-c2cc(Cl)nc(N)n2)nn1C] | 240 |
| 25 | 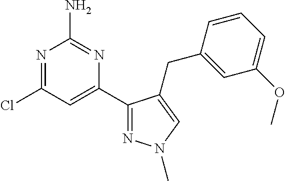 [SMILES: COc1cccc(Cc2cn(C)nc2-c2cc(Cl)nc(N)n2)c1] | 289 |
| 26 | 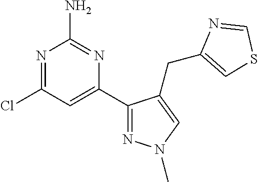 [SMILES: Cn1cc(Cc2cscn2)c(-c2cc(Cl)nc(N)n2)n1] | ~1000 |
| 27 | 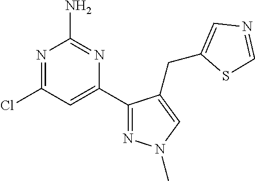 [SMILES: Cn1cc(Cc2cncs2)c(-c2cc(Cl)nc(N)n2)n1] | ~1000 |
| 28 | 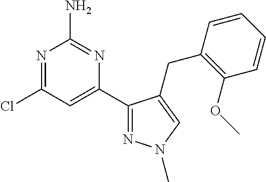 [SMILES: COc1ccccc1Cc1cn(C)nc1-c1cc(Cl)nc(N)n1] | 58 |
| 29 | 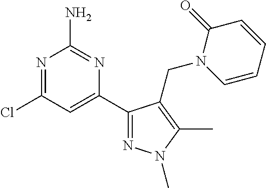 [SMILES: Cc1c(Cn2ccccc2=O)c(-c2cc(Cl)nc(N)n2)nn1C] | ~1000 |
| 30 | 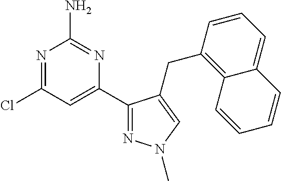 [SMILES: Cn1cc(Cc2cccc3ccccc23)c(-c2cc(Cl)nc(N)n2)n1] | 50 |
| 31 | 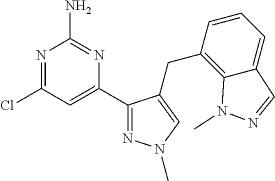 [SMILES: Cn1cc(Cc2cccc3cnn(C)c23)c(-c2cc(Cl)nc(N)n2)n1] | 250 |
| 32 | 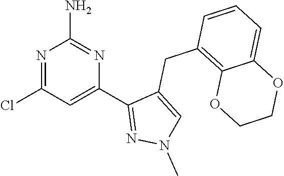 [SMILES: Cn1cc(Cc2cccc3c2OCCO3)c(-c2cc(Cl)nc(N)n2)n1] | 15 |
| 33 | 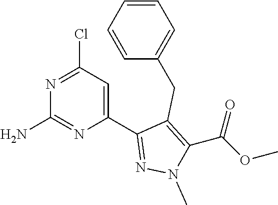 [SMILES: COC(=O)c1c(Cc2ccccc2)c(-c2cc(Cl)nc(N)n2)nn1C] | 20 |
| 34 | 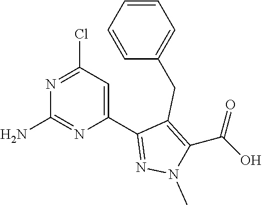 [SMILES: Cn1nc(-c2cc(Cl)nc(N)n2)c(Cc2ccccc2)c1C(=O)O] | 700 |
| 35 | 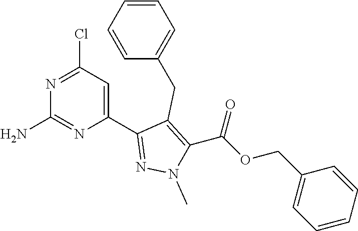 [SMILES: Cn1nc(-c2cc(Cl)nc(N)n2)c(Cc2ccccc2)c1C(=O)OCc1ccccc1] | 0.826 |
| 36 | 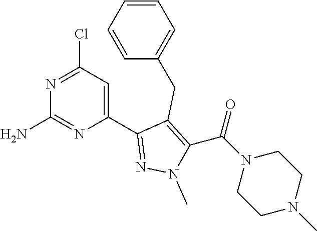 [SMILES: CN1CCN(C(=O)c2c(Cc3ccccc3)c(-c3cc(Cl)nc(N)n3)nn2C)CC1] | ~1000 |
| 37 | 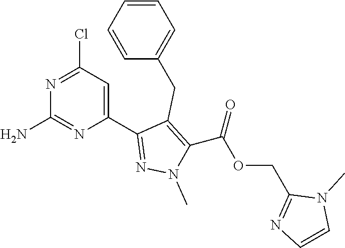 [SMILES: Cn1ccnc1COC(=O)c1c(Cc2ccccc2)c(-c2cc(Cl)nc(N)n2)nn1C] | 6 |
| 38 | 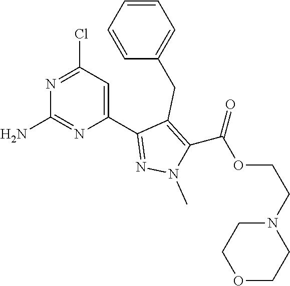 [SMILES: Cn1nc(-c2cc(Cl)nc(N)n2)c(Cc2ccccc2)c1C(=O)OCCN1CCOCC1] | 4.7 |
| 39 | 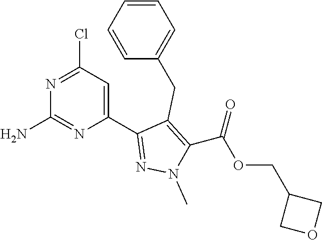 [SMILES: Cn1nc(-c2cc(Cl)nc(N)n2)c(Cc2ccccc2)c1C(=O)OCC1COC1] | 3.8 |
| 40 | 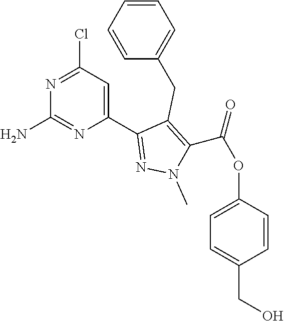 [SMILES: Cn1nc(-c2cc(Cl)nc(N)n2)c(Cc2ccccc2)c1C(=O)Oc1ccc(CO)cc1] | 1.5 |
| 41 | 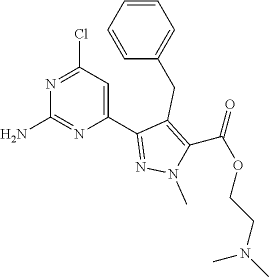 [SMILES: CN(C)CCOC(=O)c1c(Cc2ccccc2)c(-c2cc(Cl)nc(N)n2)nn1C] | 8 |
| 42 | 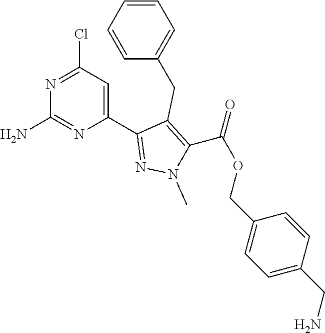 [SMILES: Cn1nc(-c2cc(Cl)nc(N)n2)c(Cc2ccccc2)c1C(=O)OCc1ccc(CN)cc1] | 3.5 |
| 43 | 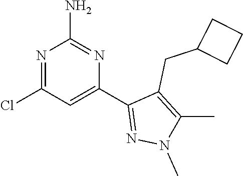 [SMILES: Cc1c(CC2CCC2)c(-c2cc(Cl)nc(N)n2)nn1C] | 100 |
| 44 | 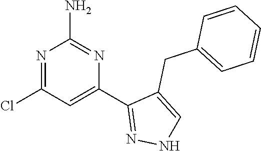 [SMILES: Nc1nc(Cl)cc(-c2n[nH]cc2Cc2ccccc2)n1] | ~5000 |
| 45 | 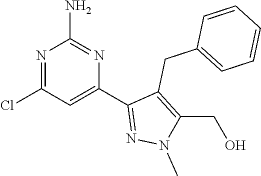 [SMILES: Cn1nc(-c2cc(Cl)nc(N)n2)c(Cc2ccccc2)c1CO] | 400 |
| 46 | 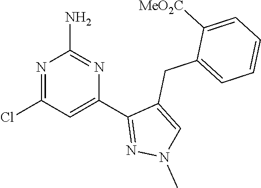 [SMILES: COC(=O)c1ccccc1Cc1cn(C)nc1-c1cc(Cl)nc(N)n1] | 22 |
| 47 | 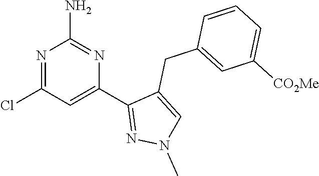 [SMILES: COC(=O)c1cccc(Cc2cn(C)nc2-c2cc(Cl)nc(N)n2)c1] | 70 |
| 48 | 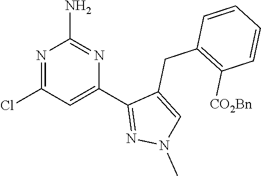 [SMILES: Cn1cc(Cc2ccccc2C(=O)OCc2ccccc2)c(-c2cc(Cl)nc(N)n2)n1] | 10 |
| 49 | 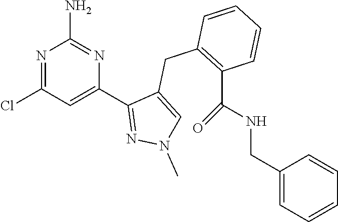 [SMILES: Cn1cc(Cc2ccccc2C(=O)NCc2ccccc2)c(-c2cc(Cl)nc(N)n2)n1] | 230 |
| 50 | 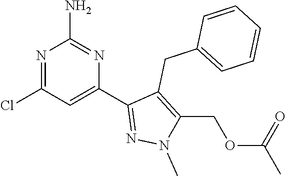 [SMILES: CC(=O)OCc1c(Cc2ccccc2)c(-c2cc(Cl)nc(N)n2)nn1C] | 310 |
| 51 | 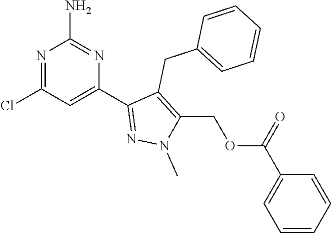 [SMILES: Cn1nc(-c2cc(Cl)nc(N)n2)c(Cc2ccccc2)c1COC(=O)c1ccccc1] | 32 |
| 52 | 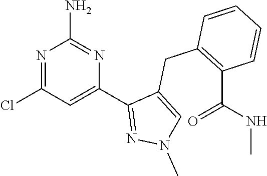 [SMILES: CNC(=O)c1ccccc1Cc1cn(C)nc1-c1cc(Cl)nc(N)n1] | 1200 |
| 53 | 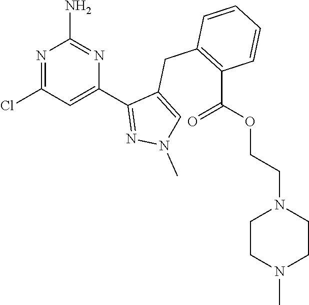 [SMILES: CN1CCN(CCOC(=O)c2ccccc2Cc2cn(C)nc2-c2cc(Cl)nc(N)n2)CC1] | 122 |
| 54 | 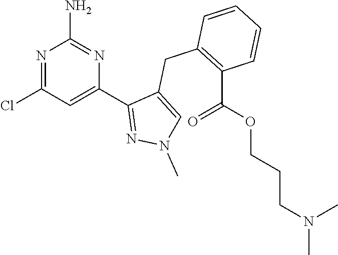 [SMILES: CN(C)CCCOC(=O)c1ccccc1Cc1cn(C)nc1-c1cc(Cl)nc(N)n1] | 93 |
| 55 | 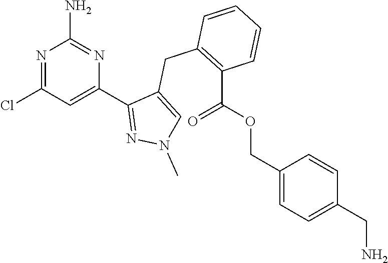 [SMILES: Cn1cc(Cc2ccccc2C(=O)OCc2ccc(CN)cc2)c(-c2cc(Cl)nc(N)n2)n1] | 11 |
| 56 | 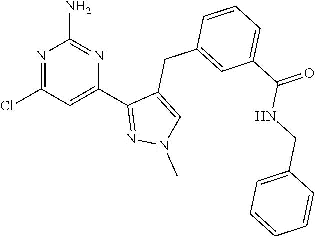 [SMILES: Cn1cc(Cc2cccc(C(=O)NCc3ccccc3)c2)c(-c2cc(Cl)nc(N)n2)n1] | 68 |
| 57 | 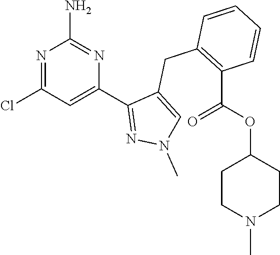 [SMILES: CN1CCC(OC(=O)c2ccccc2Cc2cn(C)nc2-c2cc(Cl)nc(N)n2)CC1] | 80 |
| 58 | 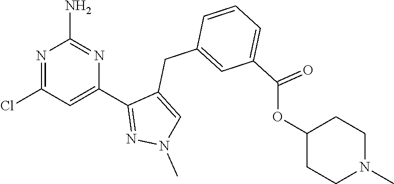 [SMILES: CN1CCC(OC(=O)c2cccc(Cc3cn(C)nc3-c3cc(Cl)nc(N)n3)c2)CC1] | 330 |
| 59 | 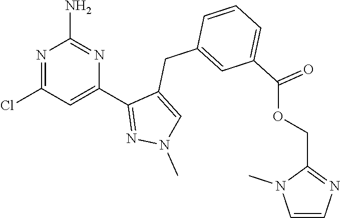 [SMILES: Cn1cc(Cc2cccc(C(=O)OCc3nccn3C)c2)c(-c2cc(Cl)nc(N)n2)n1] | 135 |
| 60 | 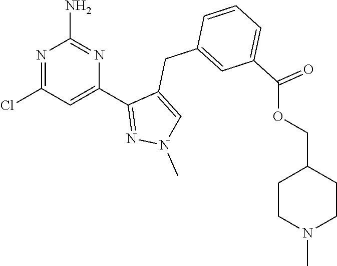 [SMILES: CN1CCC(COC(=O)c2cccc(Cc3cn(C)nc3-c3cc(Cl)nc(N)n3)c2)CC1] | 160 |
| 61 | 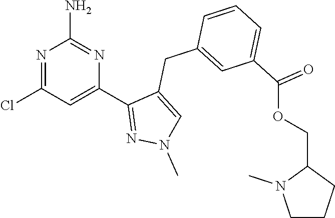 [SMILES: CN1CCCC1COC(=O)c1cccc(Cc2cn(C)nc2-c2cc(Cl)nc(N)n2)c1] | 390 |
| 62 | 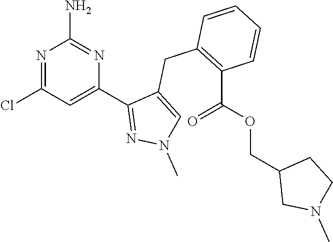 [SMILES: CN1CCC(COC(=O)c2ccccc2Cc2cn(C)nc2-c2cc(Cl)nc(N)n2)C1] | 52 |
| 63 | 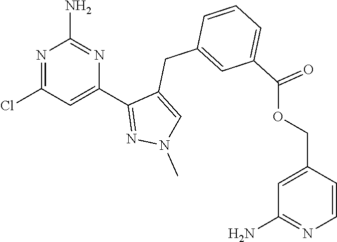 [SMILES: Cn1cc(Cc2cccc(C(=O)OCc3ccnc(N)c3)c2)c(-c2cc(Cl)nc(N)n2)n1] | 0.455 |
| 64 | 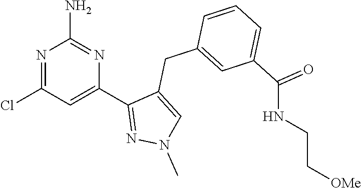 [SMILES: COCCNC(=O)c1cccc(Cc2cn(C)nc2-c2cc(Cl)nc(N)n2)c1] | ~1000 |
| 65 | 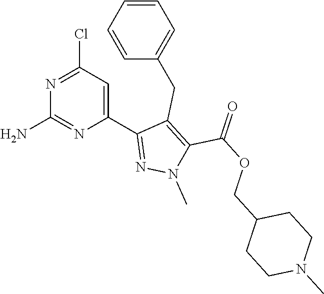 [SMILES: CN1CCC(COC(=O)c2c(Cc3ccccc3)c(-c3cc(Cl)nc(N)n3)nn2C)CC1] | 10 |
| 66 | 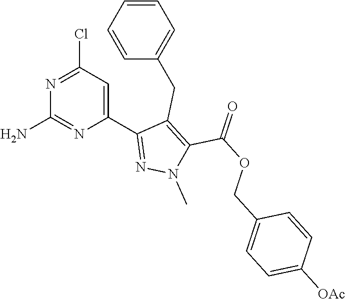 [SMILES: CC(=O)Oc1ccc(COC(=O)c2c(Cc3ccccc3)c(-c3cc(Cl)nc(N)n3)nn2C)cc1] | 26 |
| 67 | 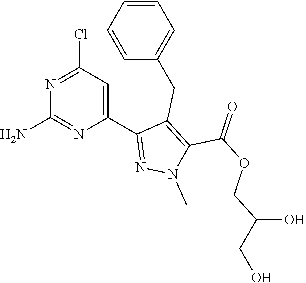 [SMILES: Cn1nc(-c2cc(Cl)nc(N)n2)c(Cc2ccccc2)c1C(=O)OCC(O)CO] | 20 |
| 68 | 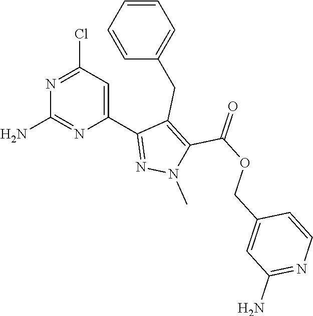 [SMILES: Cn1nc(-c2cc(Cl)nc(N)n2)c(Cc2ccccc2)c1C(=O)OCc1ccnc(N)c1] | 0.46 |
| 69 | 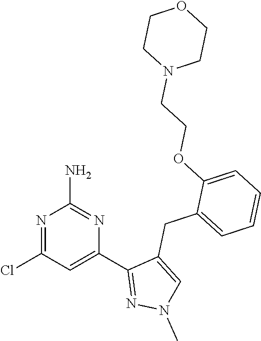 [SMILES: Cn1cc(Cc2ccccc2OCCN2CCOCC2)c(-c2cc(Cl)nc(N)n2)n1] | 4.2 |
| 70 | 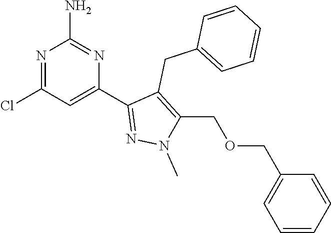 [SMILES: Cn1nc(-c2cc(Cl)nc(N)n2)c(Cc2ccccc2)c1COCc1ccccc1] | 26 |
| 71 | 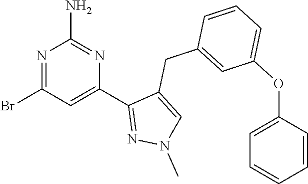 [SMILES: Cn1cc(Cc2cccc(Oc3ccccc3)c2)c(-c2cc(Br)nc(N)n2)n1] | ~1000 |
| 72 | 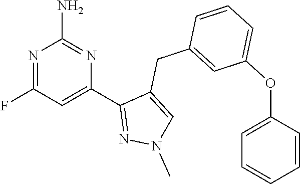 [SMILES: Cn1cc(Cc2cccc(Oc3ccccc3)c2)c(-c2cc(F)nc(N)n2)n1] | ~1000 |
| 73 | 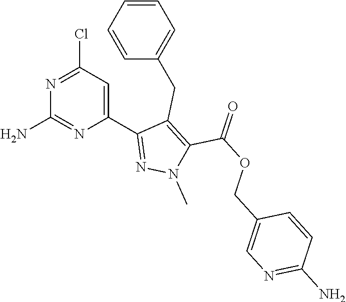 [SMILES: Cn1nc(-c2cc(Cl)nc(N)n2)c(Cc2ccccc2)c1C(=O)OCc1ccc(N)nc1] | ~1000 |
| 74 | 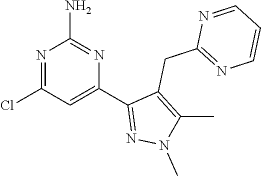 [SMILES: Cc1c(Cc2ncccn2)c(-c2cc(Cl)nc(N)n2)nn1C] | ~1000 |
| 75 | 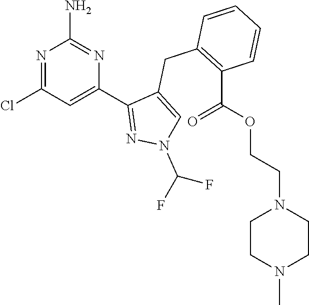 [SMILES: CN1CCN(CCOC(=O)c2ccccc2Cc2cn(C(F)F)nc2-c2cc(Cl)nc(N)n2)CC1] | 23 |
| 76 | 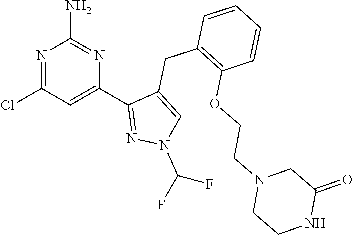 [SMILES: Nc1nc(Cl)cc(-c2nn(C(F)F)cc2Cc2ccccc2OCCN2CCNC(=O)C2)n1] | 2 |
| 77 | 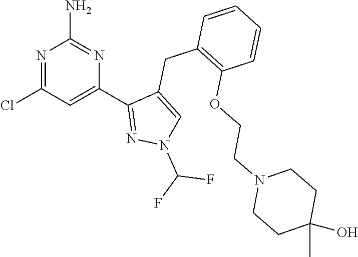 [SMILES: CC1(O)CCN(CCOc2ccccc2Cc2cn(C(F)F)nc2-c2cc(Cl)nc(N)n2)CC1] | 2.4 |
| 78 | 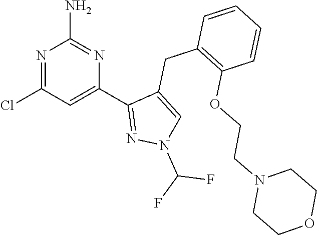 [SMILES: Nc1nc(Cl)cc(-c2nn(C(F)F)cc2Cc2ccccc2OCCN2CCOCC2)n1] | 3 |
| 79 | 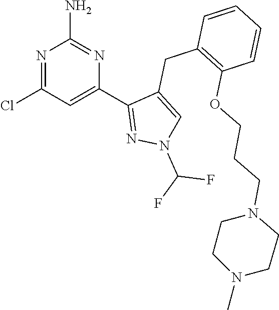 [SMILES: CN1CCN(CCCOc2ccccc2Cc2cn(C(F)F)nc2-c2cc(Cl)nc(N)n2)CC1] | 5 |
| 80 | 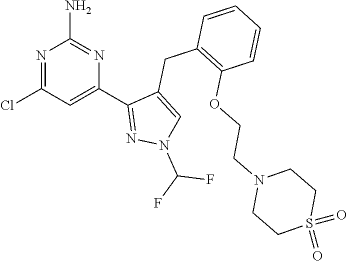 [SMILES: Nc1nc(Cl)cc(-c2nn(C(F)F)cc2Cc2ccccc2OCCN2CCS(=O)(=O)CC2)n1] | 2 |
| 81 | 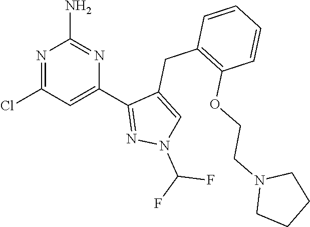 [SMILES: Nc1nc(Cl)cc(-c2nn(C(F)F)cc2Cc2ccccc2OCCN2CCCC2)n1] | 12 |
| 82 | 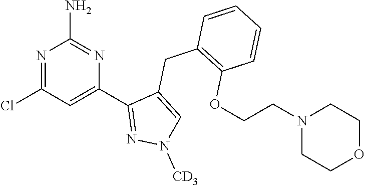 [SMILES: [2H]C([2H])([2H])n1cc(Cc2ccccc2OCCN2CCOCC2)c(-c2cc(Cl)nc(N)n2)n1] | 1.6 |
| 83 | 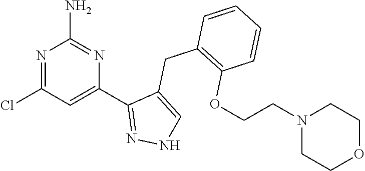 [SMILES: Nc1nc(Cl)cc(-c2n[nH]cc2Cc2ccccc2OCCN2CCOCC2)n1] | ~1000 |
| 84 | 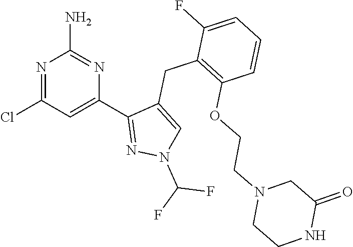 [SMILES: Nc1nc(Cl)cc(-c2nn(C(F)F)cc2Cc2c(F)cccc2OCCN2CCNC(=O)C2)n1] | 4 |
| 85 | 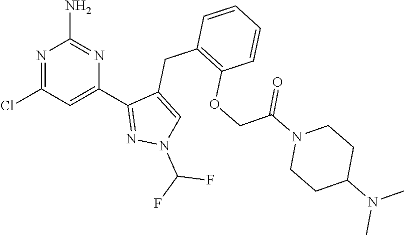 [SMILES: CN(C)C1CCN(C(=O)COc2ccccc2Cc2cn(C(F)F)nc2-c2cc(Cl)nc(N)n2)CC1] | 27 |
| 86 | 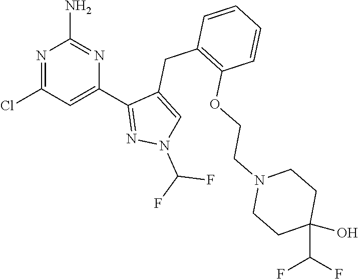 [SMILES: Nc1nc(Cl)cc(-c2nn(C(F)F)cc2Cc2ccccc2OCCN2CCC(O)(C(F)F)CC2)n1] | 3 |
| 87 | 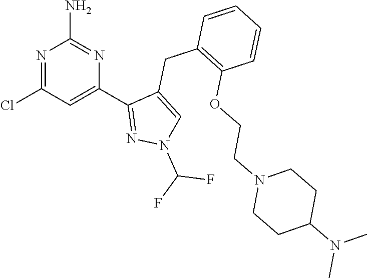 [SMILES: CN(C)C1CCN(CCOc2ccccc2Cc2cn(C(F)F)nc2-c2cc(Cl)nc(N)n2)CC1] | 12 |
| 88 | 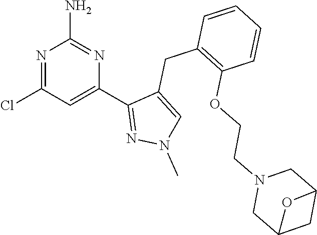 [SMILES: Cn1cc(Cc2ccccc2OCCN2CC3CC(C2)O3)c(-c2cc(Cl)nc(N)n2)n1] | 5 |
| 89 | 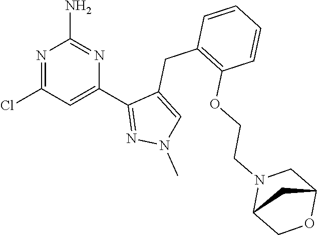 [SMILES: Cn1cc(Cc2ccccc2OCCN2C[C@H]3C[C@@H]2CO3)c(-c2cc(Cl)nc(N)n2)n1] | 6 |
| 90 | 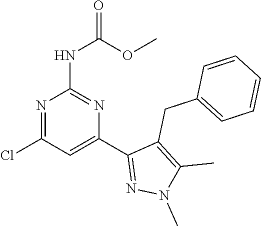 [SMILES: COC(=O)Nc1nc(Cl)cc(-c2nn(C)c(C)c2Cc2ccccc2)n1] | ~1000 |
| 91 | 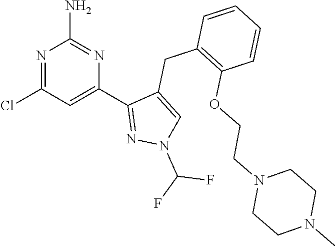 [SMILES: CN1CCN(CCOc2ccccc2Cc2cn(C(F)F)nc2-c2cc(Cl)nc(N)n2)CC1] | 18 |
| 92 | 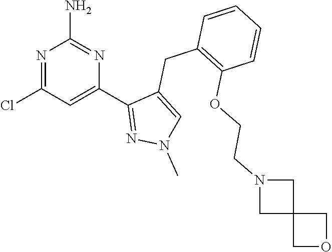 [SMILES: Cn1cc(Cc2ccccc2OCCN2CC3(COC3)C2)c(-c2cc(Cl)nc(N)n2)n1] | 7.1 |
| 93 | 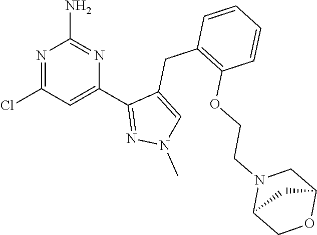 [SMILES: Cn1cc(Cc2ccccc2OCCN2C[C@@H]3C[C@H]2CO3)c(-c2cc(Cl)nc(N)n2)n1] | 10 |
| 94 | 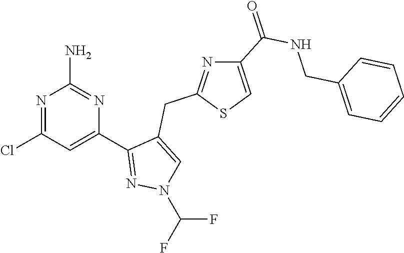 [SMILES: Nc1nc(Cl)cc(-c2nn(C(F)F)cc2Cc2nc(C(=O)NCc3ccccc3)cs2)n1] | 250 |
| 95 | 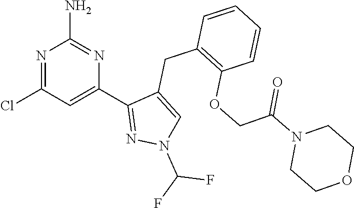 [SMILES: Nc1nc(Cl)cc(-c2nn(C(F)F)cc2Cc2ccccc2OCC(=O)N2CCOCC2)n1] | 15 |
| 96 | 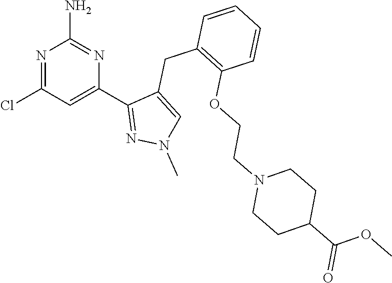 [SMILES: COC(=O)C1CCN(CCOc2ccccc2Cc2cn(C)nc2-c2cc(Cl)nc(N)n2)CC1] | 5 |
| 97 | 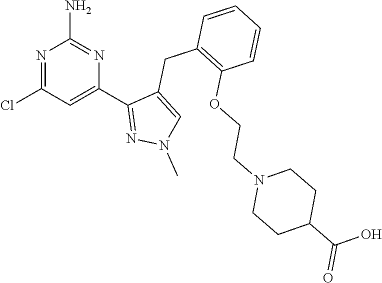 [SMILES: Cn1cc(Cc2ccccc2OCCN2CCC(C(=O)O)CC2)c(-c2cc(Cl)nc(N)n2)n1] | 250 |
| 98 | 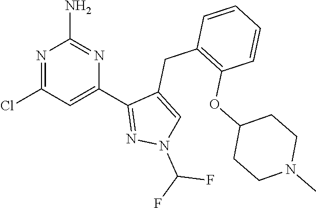 [SMILES: CN1CCC(Oc2ccccc2Cc2cn(C(F)F)nc2-c2cc(Cl)nc(N)n2)CC1] | 20 |
| 99 | 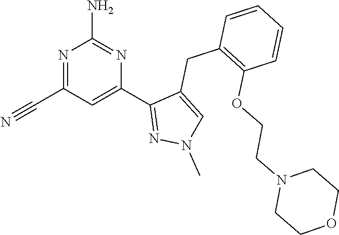 [SMILES: Cn1cc(Cc2ccccc2OCCN2CCOCC2)c(-c2cc(C#N)nc(N)n2)n1] | 100 |
| 100 | 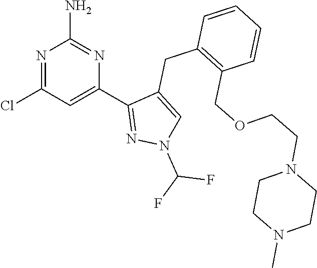 [SMILES: CN1CCN(CCOCc2ccccc2Cc2cn(C(F)F)nc2-c2cc(Cl)nc(N)n2)CC1] | 150 |
| 101 | 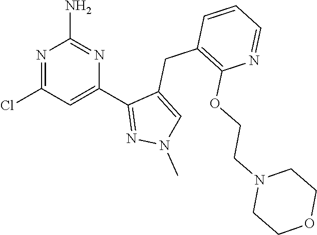 [SMILES: Cn1cc(Cc2cccnc2OCCN2CCOCC2)c(-c2cc(Cl)nc(N)n2)n1] | 150 |
| 102 | 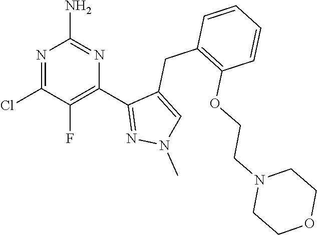 [SMILES: Cn1cc(Cc2ccccc2OCCN2CCOCC2)c(-c2nc(N)nc(Cl)c2F)n1] | 120 |
| 103 | 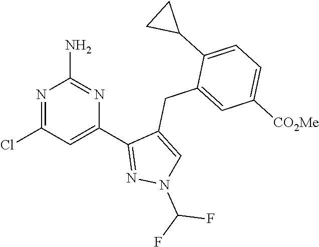 [SMILES: COC(=O)c1ccc(C2CC2)c(Cc2cn(C(F)F)nc2-c2cc(Cl)nc(N)n2)c1] | 2 |
| 104 | 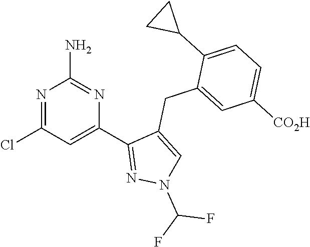 [SMILES: Nc1nc(Cl)cc(-c2nn(C(F)F)cc2Cc2cc(C(=O)O)ccc2C2CC2)n1] | 10 |
| 105 | 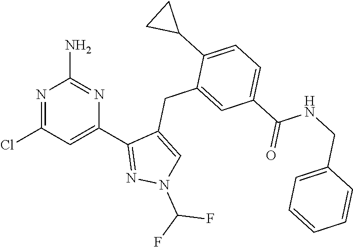 [SMILES: Nc1nc(Cl)cc(-c2nn(C(F)F)cc2Cc2cc(C(=O)NCc3ccccc3)ccc2C2CC2)n1] | 2 |
| 106 | 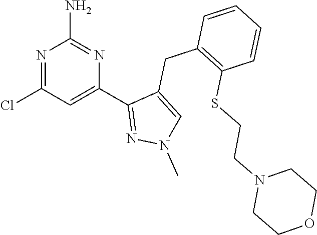 [SMILES: Cn1cc(Cc2ccccc2SCCN2CCOCC2)c(-c2cc(Cl)nc(N)n2)n1] | 60 |
| 107 | 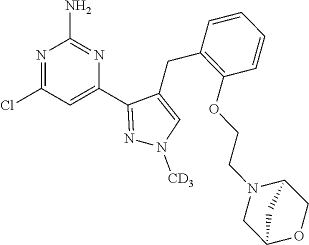 [SMILES: [2H]C([2H])([2H])n1cc(Cc2ccccc2OCCN2C[C@H]3C[C@@H]2CO3)c(-c2cc(Cl)nc(N)n2)n1] | 5 |
| 108 | 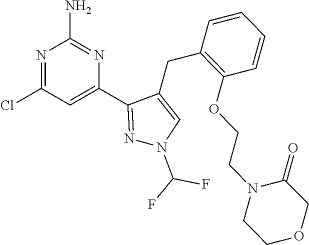 [SMILES: Nc1nc(Cl)cc(-c2nn(C(F)F)cc2Cc2ccccc2OCCN2CCOCC2=O)n1] | 5 |
| 109 | 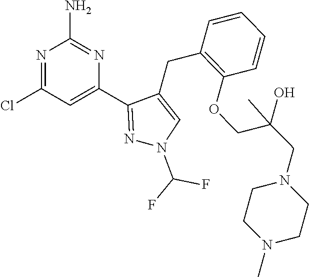 [SMILES: CN1CCN(CC(C)(O)COc2ccccc2Cc2cn(C(F)F)nc2-c2cc(Cl)nc(N)n2)CC1] | 10 |
| 110 | 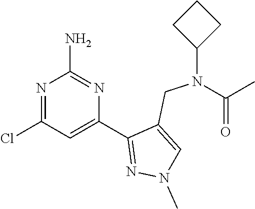 [SMILES: CC(=O)N(Cc1cn(C)nc1-c1cc(Cl)nc(N)n1)C1CCC1] | 410 |
| 111 | 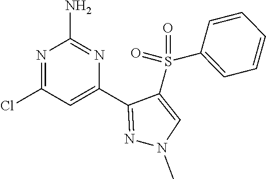 [SMILES: Cn1cc(S(=O)(=O)c2ccccc2)c(-c2cc(Cl)nc(N)n2)n1] | 3340 |
| 112 | 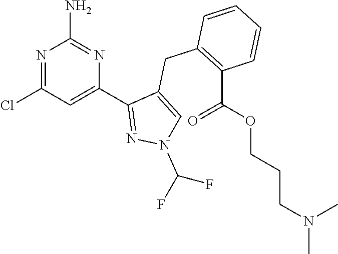 [SMILES: CN(C)CCCOC(=O)c1ccccc1Cc1cn(C(F)F)nc1-c1cc(Cl)nc(N)n1] | 74 |
| 113 | 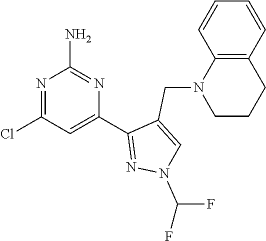 [SMILES: Nc1nc(Cl)cc(-c2nn(C(F)F)cc2CN2CCCc3ccccc32)n1] | 140 |
| 114 | 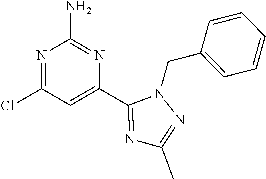 [SMILES: Cc1nc(-c2cc(Cl)nc(N)n2)n(Cc2ccccc2)n1] | 2500 |
| 115 | 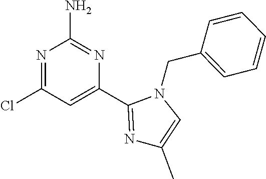 [SMILES: Cc1cn(Cc2ccccc2)c(-c2cc(Cl)nc(N)n2)n1] | ~1000 |
| 116 | 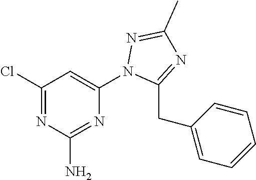 [SMILES: Cc1nc(Cc2ccccc2)n(-c2cc(Cl)nc(N)n2)n1] | 1400 |
| 117 | 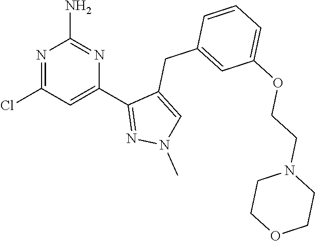 [SMILES: Cn1cc(Cc2cccc(OCCN3CCOCC3)c2)c(-c2cc(Cl)nc(N)n2)n1] | 337 |
| 118 | 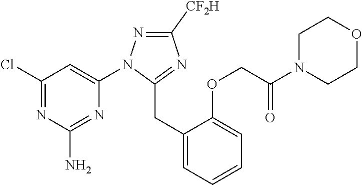 [SMILES: Nc1nc(Cl)cc(-n2nc(C(F)F)nc2Cc2ccccc2OCC(=O)N2CCOCC2)n1] | 160 |
| 119 | 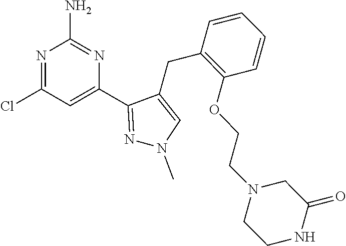 [SMILES: Cn1cc(Cc2ccccc2OCCN2CCNC(=O)C2)c(-c2cc(Cl)nc(N)n2)n1] | 12 |
| 120 | 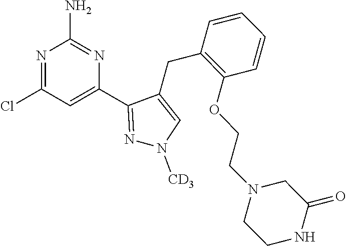 [SMILES: [2H]C([2H])([2H])n1cc(Cc2ccccc2OCCN2CCNC(=O)C2)c(-c2cc(Cl)nc(N)n2)n1] | 8 |
| 121 | 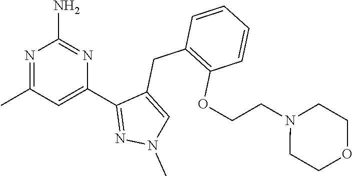 [SMILES: Cc1cc(-c2nn(C)cc2Cc2ccccc2OCCN2CCOCC2)nc(N)n1] | ~1000 |
| 122 | 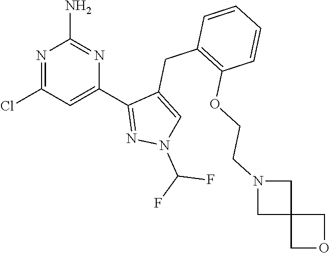 [SMILES: Nc1nc(Cl)cc(-c2nn(C(F)F)cc2Cc2ccccc2OCCN2CC3(COC3)C2)n1] | 5 |
| 123 | 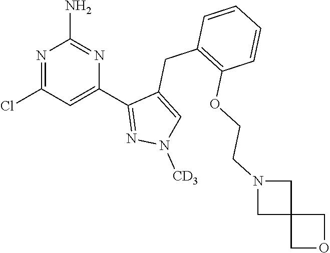 [SMILES: [2H]C([2H])([2H])n1cc(Cc2ccccc2OCCN2CC3(COC3)C2)c(-c2cc(Cl)nc(N)n2)n1] | 5 |
| 124 | 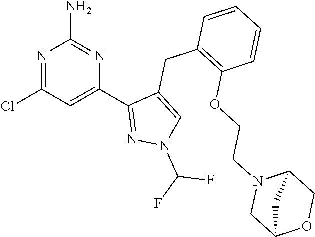 [SMILES: Nc1nc(Cl)cc(-c2nn(C(F)F)cc2Cc2ccccc2OCCN2C[C@H]3C[C@@H]2CO3)n1] | 2 |
| 125 | 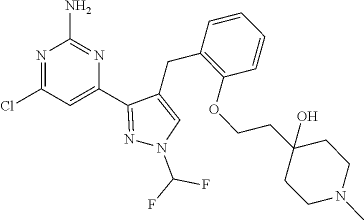 [SMILES: CN1CCC(O)(CCOc2ccccc2Cc2cn(C(F)F)nc2-c2cc(Cl)nc(N)n2)CC1] | 3 |
| 126 | 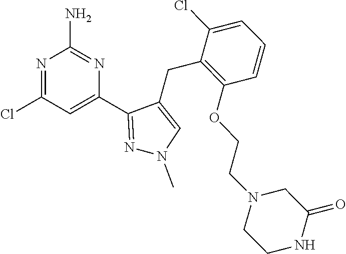 [SMILES: Cn1cc(Cc2c(Cl)cccc2OCCN2CCNC(=O)C2)c(-c2cc(Cl)nc(N)n2)n1] | 4 |
| 127 | 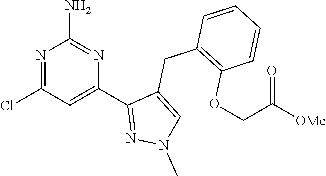 [SMILES: COC(=O)COc1ccccc1Cc1cn(C)nc1-c1cc(Cl)nc(N)n1] | 10 |
| 128 | 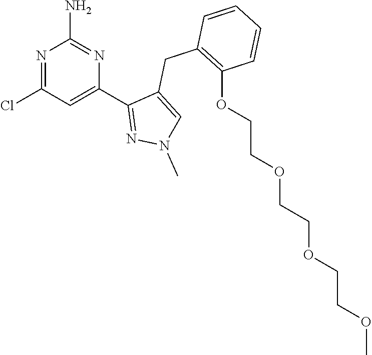 [SMILES: COCCOCCOCCOc1ccccc1Cc1cn(C)nc1-c1cc(Cl)nc(N)n1] | 15 |
| 129 | 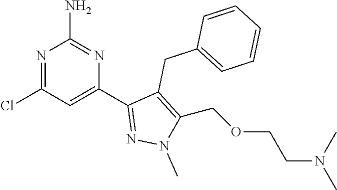 [SMILES: CN(C)CCOCc1c(Cc2ccccc2)c(-c2cc(Cl)nc(N)n2)nn1C] | 300 |
| 130 | 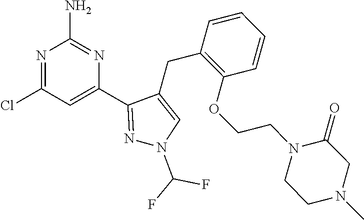 [SMILES: CN1CCN(CCOc2ccccc2Cc2cn(C(F)F)nc2-c2cc(Cl)nc(N)n2)C(=O)C1] | < 5 |
| 131 | 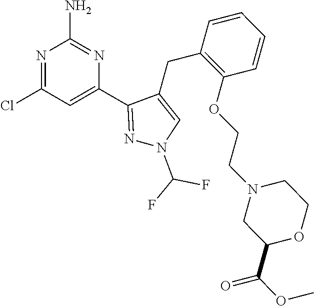 [SMILES: COC(=O)[C@H]1CN(CCOc2ccccc2Cc2cn(C(F)F)nc2-c2cc(Cl)nc(N)n2)CCO1] | >5 |
| 132 | 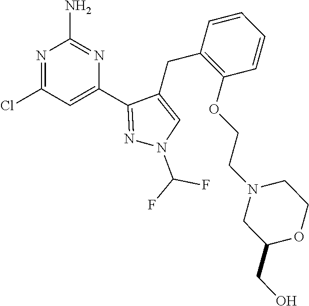 [SMILES: Nc1nc(Cl)cc(-c2nn(C(F)F)cc2Cc2ccccc2OCCN2CCO[C@@H](CO)C2)n1] | >5 |
| 133 | 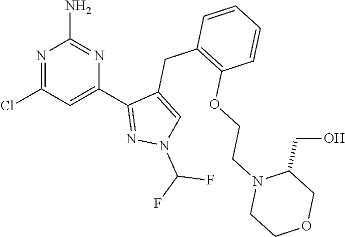 [SMILES: Nc1nc(Cl)cc(-c2nn(C(F)F)cc2Cc2ccccc2OCCN2CCOC[C@H]2CO)n1] | 3 |
| 134 | 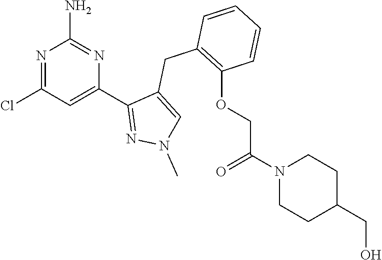 [SMILES: Cn1cc(Cc2ccccc2OCC(=O)N2CCC(CO)CC2)c(-c2cc(Cl)nc(N)n2)n1] | 15 |
| 135 | 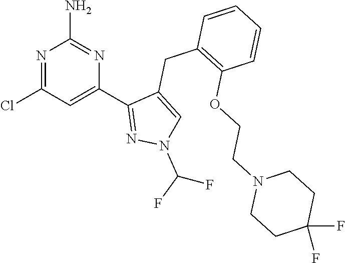 [SMILES: Nc1nc(Cl)cc(-c2nn(C(F)F)cc2Cc2ccccc2OCCN2CCC(F)(F)CC2)n1] | <5 |
| 136 | 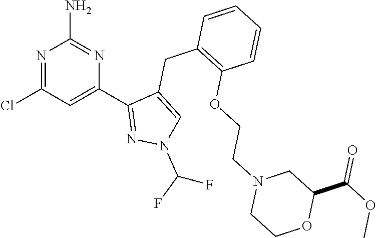 [SMILES: COC(=O)[C@@H]1CN(CCOc2ccccc2Cc2cn(C(F)F)nc2-c2cc(Cl)nc(N)n2)CCO1] | 7 |
| 137 | 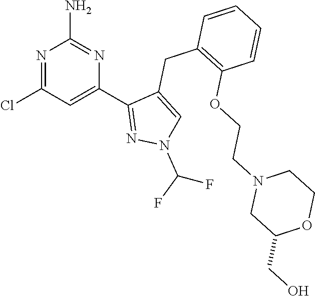 [SMILES: Nc1nc(Cl)cc(-c2nn(C(F)F)cc2Cc2ccccc2OCCN2CCO[C@H](CO)C2)n1] | 10 |
| 138 | 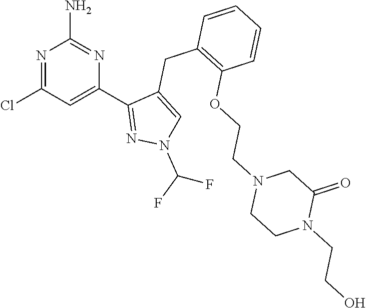 [SMILES: Nc1nc(Cl)cc(-c2nn(C(F)F)cc2Cc2ccccc2OCCN2CCN(CCO)C(=O)C2)n1] | <5 |
| 139 | 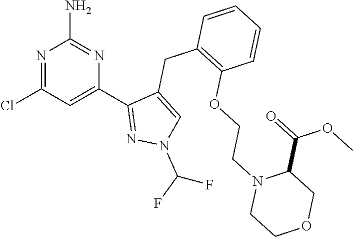 [SMILES: COC(=O)[C@H]1COCCN1CCOc1ccccc1Cc1cn(C(F)F)nc1-c1cc(Cl)nc(N)n1] | <5 |
| 140 | 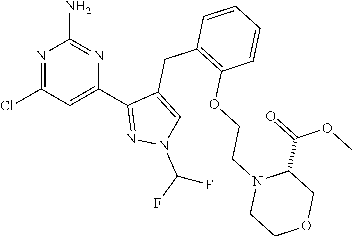 [SMILES: COC(=O)[C@@H]1COCCN1CCOc1ccccc1Cc1cn(C(F)F)nc1-c1cc(Cl)nc(N)n1] | 10 |
| 141 | 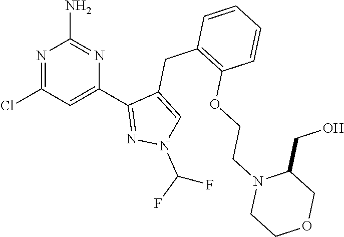 [SMILES: Nc1nc(Cl)cc(-c2nn(C(F)F)cc2Cc2ccccc2OCCN2CCOC[C@@H]2CO)n1] | <5 |
| 142 | 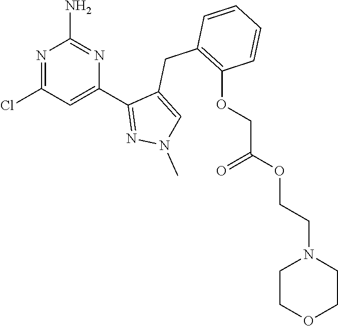 [SMILES: Cn1cc(Cc2ccccc2OCC(=O)OCCN2CCOCC2)c(-c2cc(Cl)nc(N)n2)n1] | 40 |
| 143 | 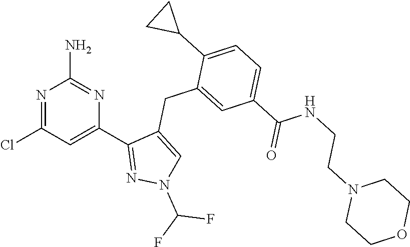 [SMILES: Nc1nc(Cl)cc(-c2nn(C(F)F)cc2Cc2cc(C(=O)NCCN3CCOCC3)ccc2C2CC2)n1] | 40 |
| 144 | 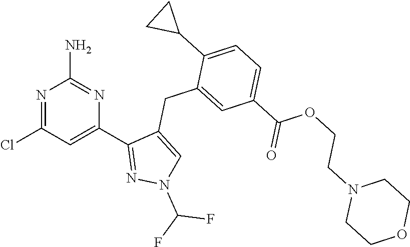 [SMILES: Nc1nc(Cl)cc(-c2nn(C(F)F)cc2Cc2cc(C(=O)OCCN3CCOCC3)ccc2C2CC2)n1] | <5 |
| 145 | 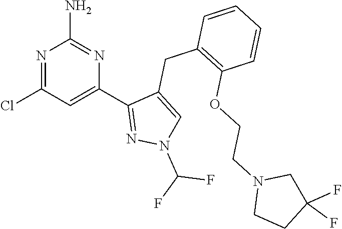 [SMILES: Nc1nc(Cl)cc(-c2nn(C(F)F)cc2Cc2ccccc2OCCN2CCC(F)(F)C2)n1] | <5 |
| 146 | 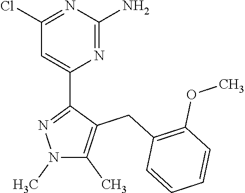 [SMILES: COc1ccccc1Cc1c(-c2cc(Cl)nc(N)n2)nn(C)c1C] | 520 |
| 147 | 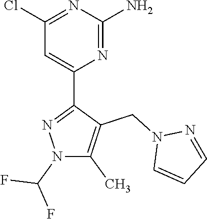 [SMILES: Cc1c(Cn2cccn2)c(-c2cc(Cl)nc(N)n2)nn1C(F)F] | 586 |
| 148 | 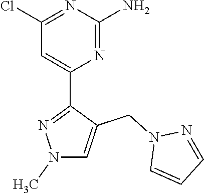 [SMILES: Cn1cc(Cn2cccn2)c(-c2cc(Cl)nc(N)n2)n1] | 378 |
| 149 | 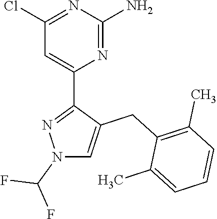 [SMILES: Cc1cccc(C)c1Cc1cn(C(F)F)nc1-c1cc(Cl)nc(N)n1] | 250 |
| 150 | 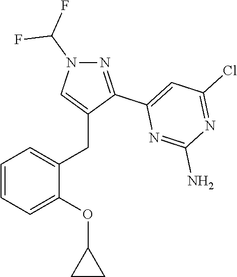 [SMILES: Nc1nc(Cl)cc(-c2nn(C(F)F)cc2Cc2ccccc2OC2CC2)n1] | 550 |
| 151 | 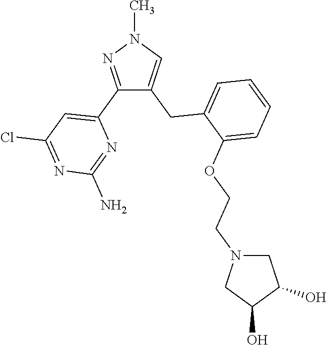 [SMILES: Cn1cc(Cc2ccccc2OCCN2C[C@H](O)[C@@H](O)C2)c(-c2cc(Cl)nc(N)n2)n1] | 30 |
| 152 | 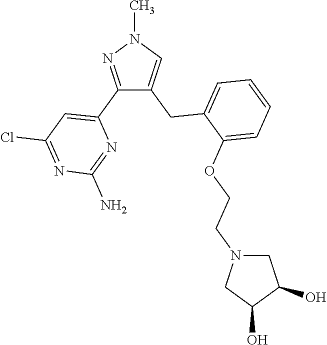 [SMILES: Cn1cc(Cc2ccccc2OCCN2C[C@@H](O)[C@@H](O)C2)c(-c2cc(Cl)nc(N)n2)n1] | 32 |
| 153 | 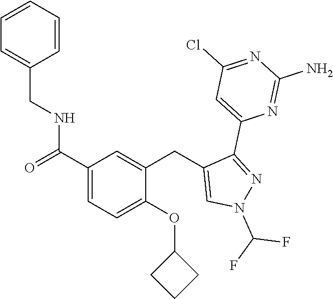 [SMILES: Nc1nc(Cl)cc(-c2nn(C(F)F)cc2Cc2cc(C(=O)NCc3ccccc3)ccc2OC2CCC2)n1] | 300 |
| 154 | 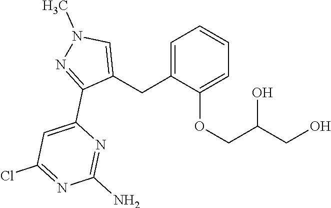 [SMILES: Cn1cc(Cc2ccccc2OCC(O)CO)c(-c2cc(Cl)nc(N)n2)n1] | 50 |
| 155 | 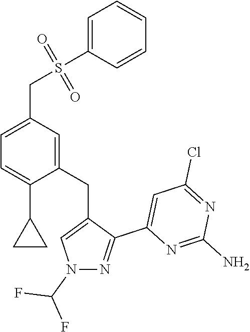 [SMILES: Nc1nc(Cl)cc(-c2nn(C(F)F)cc2Cc2cc(CS(=O)(=O)c3ccccc3)ccc2C2CC2)n1] | <5 |
| 156 | 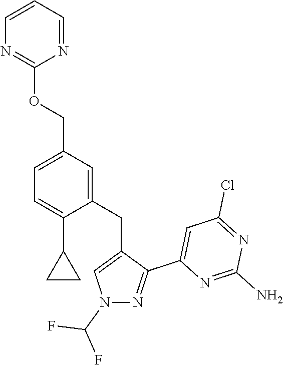 [SMILES: Nc1nc(Cl)cc(-c2nn(C(F)F)cc2Cc2cc(COc3ncccn3)ccc2C2CC2)n1] | <5 |
| 157 | 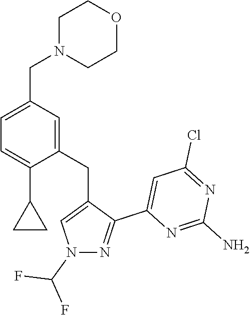 [SMILES: Nc1nc(Cl)cc(-c2nn(C(F)F)cc2Cc2cc(CN3CCOCC3)ccc2C2CC2)n1] | 6 |
| 158 | 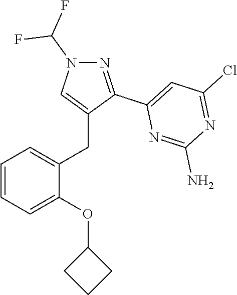 [SMILES: Nc1nc(Cl)cc(-c2nn(C(F)F)cc2Cc2ccccc2OC2CCC2)n1] | 20 |
| 159 | 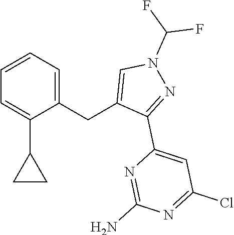 [SMILES: Nc1nc(Cl)cc(-c2nn(C(F)F)cc2Cc2ccccc2C2CC2)n1] | 50 |
| 160 | 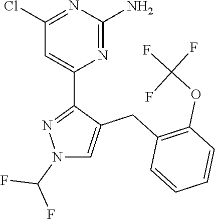 [SMILES: Nc1nc(Cl)cc(-c2nn(C(F)F)cc2Cc2ccccc2OC(F)(F)F)n1] | 60 |
| 161 | 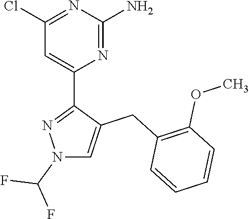 [SMILES: COc1ccccc1Cc1cn(C(F)F)nc1-c1cc(Cl)nc(N)n1] | 16 |
| 162 | 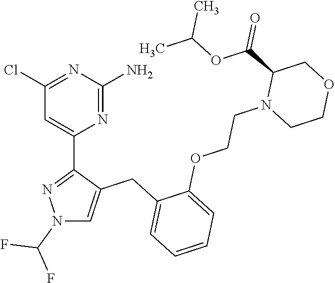 [SMILES: CC(C)OC(=O)[C@H]1COCCN1CCOc1ccccc1Cc1cn(C(F)F)nc1-c1cc(Cl)nc(N)n1] | <5 |
| 163 | 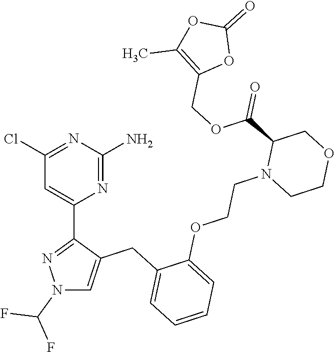 [SMILES: Cc1oc(=O)oc1COC(=O)[C@H]1COCCN1CCOc1ccccc1Cc1cn(C(F)F)nc1-c1cc(Cl)nc(N)n1] | 12 |
| 164 | 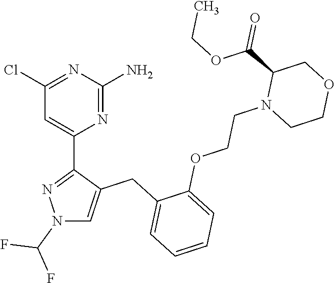 [SMILES: CCOC(=O)[C@H]1COCCN1CCOc1ccccc1Cc1cn(C(F)F)nc1-c1cc(Cl)nc(N)n1] | 8 |
| 165 | 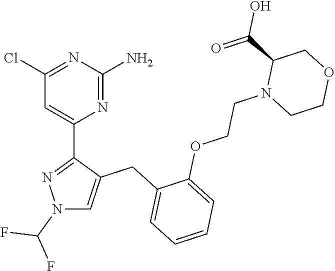 [SMILES: Nc1nc(Cl)cc(-c2nn(C(F)F)cc2Cc2ccccc2OCCN2CCOC[C@@H]2C(=O)O)n1] | <5 |
| 166 | 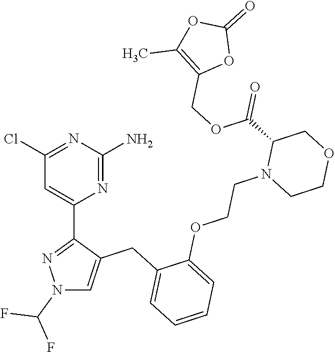 [SMILES: Cc1oc(=O)oc1COC(=O)[C@@H]1COCCN1CCOc1ccccc1Cc1cn(C(F)F)nc1-c1cc(Cl)nc(N)n1] | 50 |
| 167 | 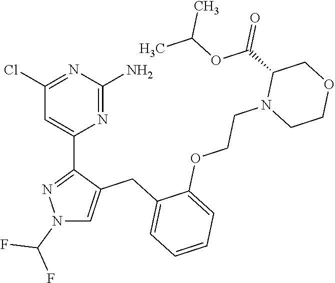 [SMILES: CC(C)OC(=O)[C@@H]1COCCN1CCOc1ccccc1Cc1cn(C(F)F)nc1-c1cc(Cl)nc(N)n1] | 23 |
| 168 | 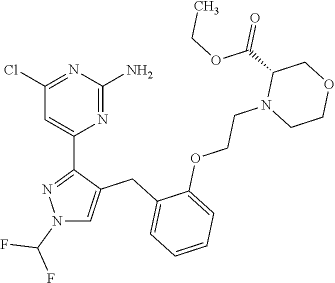 [SMILES: CCOC(=O)[C@@H]1COCCN1CCOc1ccccc1Cc1cn(C(F)F)nc1-c1cc(Cl)nc(N)n1] | <5 |
| 169 | 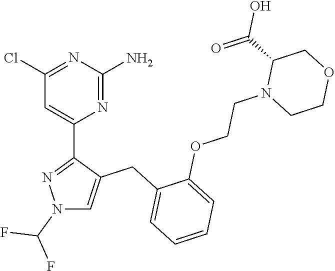 [SMILES: Nc1nc(Cl)cc(-c2nn(C(F)F)cc2Cc2ccccc2OCCN2CCOC[C@H]2C(=O)O)n1] | 40 |
| 170 | 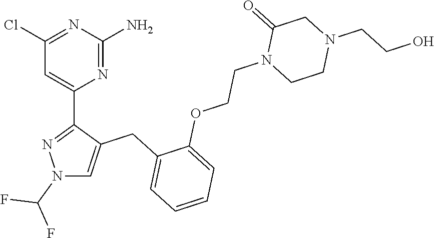 [SMILES: Nc1nc(Cl)cc(-c2nn(C(F)F)cc2Cc2ccccc2OCCN2CCN(CCO)CC2=O)n1] | 10 |
| 171 | 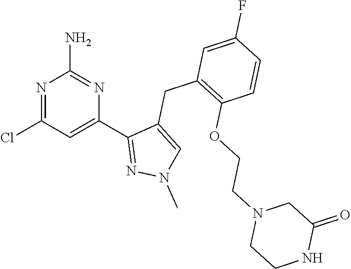 [SMILES: Cn1cc(Cc2cc(F)ccc2OCCN2CCNC(=O)C2)c(-c2cc(Cl)nc(N)n2)n1] | 25 |
| 172 | 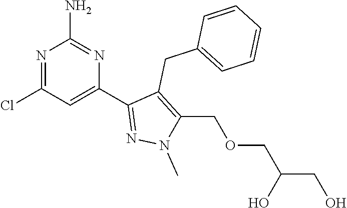 [SMILES: Cn1nc(-c2cc(Cl)nc(N)n2)c(Cc2ccccc2)c1COCC(O)CO] | 350 |
| 173 | 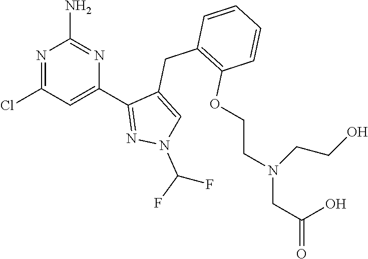 [SMILES: Nc1nc(Cl)cc(-c2nn(C(F)F)cc2Cc2ccccc2OCCN(CCO)CC(=O)O)n1] | 8 |
| 174 | 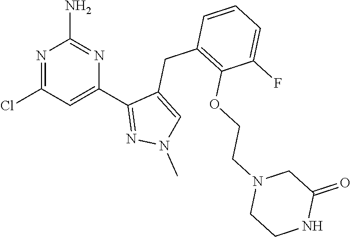 [SMILES: Cn1cc(Cc2cccc(F)c2OCCN2CCNC(=O)C2)c(-c2cc(Cl)nc(N)n2)n1] | 25 |
| 175 | 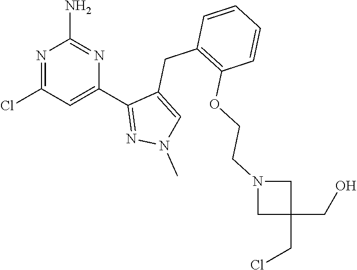 [SMILES: Cn1cc(Cc2ccccc2OCCN2CC(CO)(CCl)C2)c(-c2cc(Cl)nc(N)n2)n1] | 20 |
| 176 | 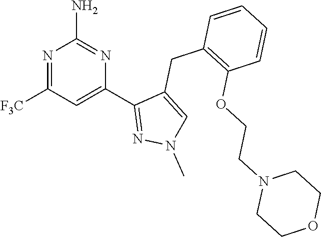 [SMILES: Cn1cc(Cc2ccccc2OCCN2CCOCC2)c(-c2cc(C(F)(F)F)nc(N)n2)n1] | 1732 |
| 177 | 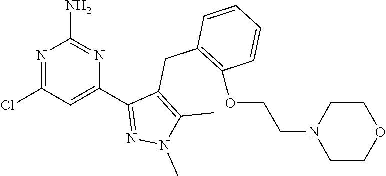 [SMILES: Cc1c(Cc2ccccc2OCCN2CCOCC2)c(-c2cc(Cl)nc(N)n2)nn1C] | 32 |
| 178 | 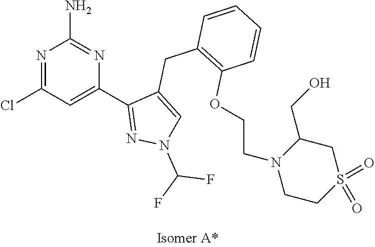 [SMILES: Nc1nc(Cl)cc(-c2nn(C(F)F)cc2Cc2ccccc2OCCN2CCS(=O)(=O)CC2CO)n1] | 80 |
| 179 | 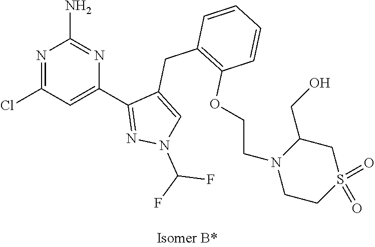 [SMILES: Nc1nc(Cl)cc(-c2nn(C(F)F)cc2Cc2ccccc2OCCN2CCS(=O)(=O)CC2CO)n1] | 690 |
| 180 | 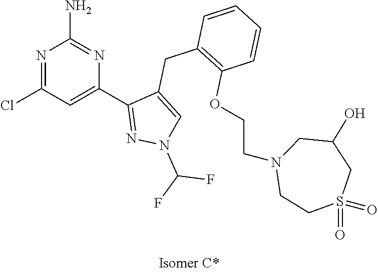 [SMILES: Nc1nc(Cl)cc(-c2nn(C(F)F)cc2Cc2ccccc2OCCN2CCS(=O)(=O)CC(O)C2)n1] | 467 |
| 181 | 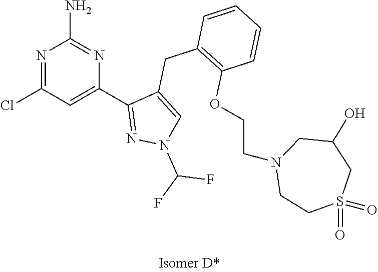 [SMILES: Nc1nc(Cl)cc(-c2nn(C(F)F)cc2Cc2ccccc2OCCN2CCS(=O)(=O)CC(O)C2)n1] | 30 |

*Isomers A and B are enantiomers; Isomers C and D are enantiomers

Cellular cAMP Accumulation Assay

4-4 cells were generated and functionally authenticated in our laboratory as previously described (Zippin et al., (2013) CO₂/HCO₃(−)- and calcium-regulated soluble adenylyl cyclase as a physiological ATP sensor. J Biol Chem 288, 33283-91) and grown in DMEM+10% FBS. 1.25×10^{6 4}-4 cells were seeded per well of a 24 well plate and incubated for 24 hours at 37° C., 5% CO₂. One hour before the experiment the media was aspirated and replaced with 300 μl fresh media. For 5 min, in duplicate wells, cells were preincubated with sAC inhibitor at the indicated concentrations or DMSO as control. For cAMP accumulation, cells were incubated with 500 μM IBMX for 5 min. To stop the reaction and to lyse the cells, the media was aspirated and replaced with 250 μl 0.1 M HCL. After shaking the plate for 5 min the cell lysate was transferred to a fresh tube and centrifuged at 1000×g for 5 min. The supernatant was used for cAMP quantification using the Direct cAMP Elisa kit (Enzo) following the manufacturer's instructions. Data for representative examples is shown in Table B.

TABLE B
Cellular cAMP Accumulation Study

| Example No. | Cell IC₅₀ (nM) |
|---|---|
| 16 | 196 |
| 4 | 413 |
| 2 | 154 |
| 9 | 194 |
| 10 | 105 |
| 11 | 411 |
| 14 | 421 |
| 1 | 102 |
| 33 | 67 |
| 35 | 4.6 |
| 46 | 51 |
| 21 | 255 |
| 47 | 323 |
| 53 | 4.8 |
| 54 | 25 |
| 41 | 5.6 |
| 57 | 13 |
| 69 | 14 |
| 75 | 13 |
| 76 | 4.4 |
| 77 | 12 |
| 78 | 4.1 |
| 82 | 2.6 |
| 119 | 7.1 |
| 89 | 3.8 |
| 120 | 17 |
| 92 | 16 |
| 93 | 16 |
| 122 | 13 |
| 123 | 35 |
| 124 | 9.8 |
| 103 | 13 |
| 128 | 259 |
| 132 | 12 |
| 133 | 7 |
| 137 | 22 |
| 139 | 3.3 |
| 140 | 24 |
| 141 | 7.7 |
| 156 | 20 |
| 157 | 6.84 |
| 158 | 301 |
| 159 | 126.2 |
| 161 | 118.4 |

### Male and Female Contraception

### sAC Inhibitors for Contraceptive Uses

Efforts to develop new male or female non-hormonal, orally available contraceptives assume that to be effective and safe, targets must be (1) essential for fertility; (2) amenable to targeting by small-molecule inhibitors; and (3) restricted to the germline. As described herein, the third assumption was questioned and it was proposed that despite its wide expression, soluble adenylyl cyclase (sAC: ADCY10), which is essential for male fertility, is a valid target. It was hypothesized that an acute-acting sAC inhibitor may provide orally available, on-demand, non-hormonal contraception for men without adverse, mechanism-based effects.

### A Novel Strategy for Male Contraception

With existing contraceptive options, preventing unintended pregnancies is largely the responsibility of females, for which several options exist. Female methods with greater than 99% success rates include tubal ligation, which is permanent, and intrauterine devices or hormonal implants, which require insertion by a doctor [Reference 1]. User-controlled barrier methods for females (i.e., diaphragms, sponges, or spermicides) result in failure rates greater than 13%. Finally, the only orally delivered methods available are hormone-based pills exclusively for women. Oral contraceptives require long-term use, carry significant side effects that are not easily tolerated by many women, and have failure rates up to 4-7%. In stark contrast, men have only two real choices: surgical vasectomy and condoms. Vasectomy has failure rates as low as 0.15% and is extremely effective, but it is largely irreversible [Reference 1] and therefore unsuitable for many men. On the other end of the spectrum, condoms supply on-demand contraception, but largely due to improper use, they have a typical failure rate of 13% and suffer from compliance issues; men (or couples) often report disliking their use due to discomfort or inconvenience [Reference 2].

Despite these drawbacks, condoms have been widely used since the time of the Roman Empire, which means that, except for surgery, male contraception has not meaningfully advanced for 2000 years [References 2, 3]. Thus, there is a profound need for new contraceptive strategies with an emphasis on non-hormonal methods and an even greater emphasis on methods enlisting males. Up till now, efforts to develop a male contraceptive focused exclusively on targets addressing three key questions: (i) Is it essential for spermatozoa development or function?; (ii) Can it be blocked with specific and reversible pharmacological agents?; and (iii) Is it exclusively functioning in the male germ cell? The final criteria was believed essential to ensure that the target could be safely blocked without any adverse, mechanism-based side effects. However, described herein is a viable alternative: a strategy where a reversible pharmacological agent against a target that satisfies only the first two criteria may be able to provide safe and effective, orally available, non-hormonal, “on-demand” contraception for men.

### Soluble Adenylyl Cyclase is a Unique Enzyme Essential for Male Fertility in Mice and Humans

Cyclic AMP (cAMP) is a nearly universally utilized second messenger molecule mediating signals throughout the bacterial and animal kingdoms. cAMP is synthesized by a broad family of adenylyl cyclases, and mammals possess two distinct classes of adenylyl cyclases: transmembrane adenylyl cyclases (tmACs) and soluble adenylyl cyclase (sAC) [Reference 4]. The tmACs are regulated by heterotrimeric G proteins and mediate cellular responses to intercellular signals, including hormones and neurotransmitters. For decades, the well-characterized family of tmACs (ADCY1-ADCY9) were thought to be the sole sources of cAMP in mammalian cells. Prior to its molecular isolation [Reference 5], all studies concerning sAC were, by necessity, based on following its biochemical activity. From these studies, soluble AC activity was predicted to be present only in testis [Reference 6]; specifically, it was postulated to be restricted to male germ cells. Its activity first appeared concomitantly with the development of spermatids in rats [References 7, 8] and humans [Reference 9], was missing in testicular feminized rats which contain little or no haploid germ cells [Reference 10], and was present in testis fractions enriched for spermatids [References 8, 10]. A biochemically-related activity was detected in spermatozoa, and its activity was thought to be stimulated by sodium bicarbonate [References 11-14]. In 1999, sAC was successfully cloned and purified (ADCY10), defining a distinct adenylyl cyclase family in mammals [Reference 5].

A 50 kDa isoform of sAC from rat testis was purified, which enabled isolating ADCY10 cDNAs encoding the full-length isoform of sAC (sACₙ) [Reference 5]. At its amino terminus, two related nucleotidyl cyclase catalytic domains form a generic Class III AC catalytic core, which is necessary and sufficient for catalytic activity. Following the catalytic region is a long carboxy-terminus whose function remains largely unexplored. This carboxy terminus contains an autoinhibitory domain [Reference 15], a heme-binding domain [Reference 16], and based on weak sequence similarities, a putative STAND module [Reference 17]. However, how these presumptive regulatory domains modulate sAC activity remains unknown. Alternative splicing results in a premature stop codon [Reference 18] to generate a ‘truncated’ sAC isoform (sACₜ) comprising the two catalytic domains that correspond to the ˜50 kDa isoform purified from testis. Heterologous expression of the cloned sAC transcripts [References 5, 18, 19] and purification of the heterologously expressed sACₜ [References 20, 21] protein clarified the biochemical distinctions between sAC and tmACs (reviewed in [Reference 4]). While insensitive to the known activators of tmACs, heterotrimeric G proteins [Reference 8] and forskolin [Reference 22], sAC activity is uniquely stimulated by bicarbonate, which accelerates substrate turnover [References 20, 21]. Crystal structures of the catalytic domain of human sAC and its complexes with substrates, products, bicarbonate, and analogs revealed the bicarbonate binding site (BBS) and identified local rearrangements contributing to activation [Reference 23]. The sAC BBS is analogous to the forskolin binding site in tmACs, defining this as a general, regulatory site in mammalian adenylyl cyclases and providing a structural basis for the activator selectivity between sAC and tmACs. Forskolin, which is inert on sAC [References 5, 24], does not fit into sAC's tighter, positively charged BBS [Reference 23], and bicarbonate does not bind to the wide, hydrophobic tmAC site lacking the bicarbonate recognizing residues. sAC is also regulated by calcium, which modulates the enzyme's affinity for substrate ATP [References 19, 21], and its catalytic activity is sensitive to physiologically relevant changes in cellular ATP levels [References 21, 25].

In sperm, sAC is the major cAMP-generating enzyme, crucial for sperm motility and capacitation (reviewed in [References 26, 27]). Capacitation is the essential maturation process required for sperm to acquire fertilization competence; it commences upon ejaculation and continues as sperm transit through the female tract [References 28, 29]. Upon leaving the testes, mammalian sperm are morphologically mature, but unable to fertilize an oocyte. They are stored in the cauda region of the epididymis in an environment characterized by low pH (i.e., 6.5-6.8 instead of 7.4) and low HCO₃ concentration (i.e., 2-7 mM instead of 25 mM) [30]. This unique epididymal luminal environment maintains the sperm in a dormant state. Upon ejaculation, sperm come into contact with the high HCO₃ and Ca²¹ concentrations present in seminal fluid [References 31, 32], which synergize to activate sAC [References 19, 21, 33, 34]. The activation of sAC rapidly (i.e., within seconds) elevates sperm cAMP which increases the flagellar beat frequency more than 2-fold [Reference 35]. Two independently generated strains of mice with ADCY10 knocked out (KO) exhibit male-specific sterility [References 35-37]; sAC-deficient sperm lack cAMP synthesis, are immotile, and do not display molecular hallmarks normally accompanying capacitation [References 37, 38]. Recently, this phenotype was identified in humans. In 2019, two infertile male patients were reported who were homozygous for a frameshift mutation in the exonic region of ADCY10, leading to premature termination and interruption of the catalytic domains [Reference 39]. Similar to sAC null mice, sperm from those patients are immotile, and this motility defect could be rescued with cell-permeable cAMP analogs. Thus, sAC satisfies the first criteria as a potential target for a male contraceptive: It is essential in sperm for male fertility in mice and men.

### sAC can be Selectively and Reversibly Inhibited by Small Molecules

Following the molecular identification of sAC, to be able to spatially and temporally probe its functions, ligands that modulate sAC without affecting tmACs were required. The first known sAC inhibitors were catechol estrogens (CE), which were found to inhibit non-competitively through binding to a groove near the active site and chelating a divalent cation essential for adenylyl cyclase activity [Reference 40]. While CEs demonstrated an ability to selectively inhibit sAC in cellular systems [References 41, 42], they are not specific for sAC relative to tmACs [Reference 40]. To satisfy the need for sAC-selective pharmacological tools, the sAC-specific inhibitor KH7 was identified in a small molecule high throughput screen (HTS) [Reference 37]. KH7 is inert against tmACs, and it is cell-permeable and inhibits sAC in tissues and animals [Reference 37, 43]. KH7 has grown into the most widely used pharmacological agent for identifying sAC functions [Reference 44], including blocking sperm capacitation and in vitro fertilization (IVF) [Reference 37]. Despite its widespread use, KH7 has liabilities that result in sAC-independent toxicity [Reference 45]. In a subsequent HTS using human sAC, the chemically distinct, sAC-specific inhibitor LRE1 was identified [Reference 46]; LRE1 also blocks the sAC-mediated functions in sperm. Thus, two structurally distinct inhibitors block sAC-dependent functions in sperm essential for fertilization, confirming the second criteria for developing a male contraceptive; sAC is amenable to targeting by small-molecule inhibitors.

### sAC is Widely Expressed

Soon after the molecular isolation of sAC, it became clear that the third criteria for a male contraceptive posed significant challenges. Up to that point, biochemical characterization of soluble adenylyl cyclase activity suggested that sAC expression was restricted to male germ cells, and initial Northern Blot, RT-PCR, and in situ hybridization experiments confirmed that sAC expression was indeed highest in male germ cells [References 5, 47]. However, these and other studies [References 20, 48, 49] revealed that sAC is also widely expressed, albeit at low levels. And consistent with widespread distribution, genetic and pharmacological experiments identified a role for sAC in a number of physiological processes in addition to male fertility (reviewed in [References 44, 50-52]). For example, sAC in somatic tissues mediates the cAMP-dependent signaling cascades which regulate luminal pH in the epididymis [Reference 42]; ciliary beat frequency in airway epithelia in response to elevated CO₂ [References 53, 54]; regulation of intraocular pressure [References 43, 55]; and leukocyte migration [Reference 56].

These somatic functions were assumed to complicate sAC's contraceptive potential. However, the two infertile male patients homozygous for inactivating mutations in sAC are healthy adults; besides infertility, their only reported health issue is increased incidence of kidney stones [Reference 39]. Similarly, the sole overt phenotype in the two molecularly distinct sAC KO mouse strains is male-specific sterility [References 35-37]. Other phenotypes observed in sAC KO mice (reviewed in [Reference 44]) and men [Reference 39] are conditional (i.e., decreased airway ciliary beat frequency in response to elevated CO₂), or they are not expected to be detrimental when transiently induced (i.e., increased risk of kidney stones, increased intraocular pressure, decreased leukocyte migration). Thus, although sAC is widely expressed, the effects of its loss are primarily restricted to male infertility, and it appears that somatic functions of sAC-generated cAMP are likely to be tolerated if sAC function is acutely inhibited.

The example of another widely expressed gene that remains safe even when systemically targeted was also considered. PDE5 is expressed in multiple tissues [Reference 57], yet sildenafil and vardenafil, which are acute PDE5 inhibitors (half-lives 4-5 hours), are sufficiently safe for treating erectile dysfunction. These PDE5 inhibitors teach that acute inhibition can be markedly different from chronic loss. Thus, it was proposed that by carefully controlling the time and dose of a fast-acting, reversible sAC inhibitor, acute administration can provide on-demand, reversible, effective contraception without adverse, mechanism-based effects.

### Rational Design of Acute Inhibitors of sAC to Provide On-Demand Contraception

TmACs are the enzymes most closely related to sAC in mammalian genomes; thus, selective sAC-inhibitors must be inert against tmACs. Active site differences between sAC and tmACs are subtle, making it an improbable site for selective inhibitors. In contrast, because only sAC is regulated by bicarbonate [References 20, 21, 23, 58], sAC's allosteric BBS has potential as a site for sAC-specific inhibitors. A first compound studied for exploiting the sAC-specific BBS was 4,4′-diisothiocyanatostilbene-2,2′-disulfonic acid, a bicarbonate transporter blocker that was speculated to enter the BBS with one of its sulfonic acid moieties. A sAC complex structure revealed, however, that it binds at the active site entrance, blocking access to the active site and BBS [Reference 23]. Thus far, three small molecules were structurally identified to occupy the BBS: (1) ASI-8 occupies the BBS and extends into the active site [Reference 59]; (2) the organochloride bithionol occupies the mostly hydrophobic BBS access channel for a mixed-type inhibition with respect to ATP and positions a chlorine in the bicarbonate pocket [Reference 60]; and (3) LRE1. Crystal structures of sAC/LRE1 complexes revealed that the compound's 2-amino-6-chloropyrimidine occupies the BBS and its small cyclopropyl moiety reaches into the channel connecting BBS and active site but does not overlap with ATP binding regions [Reference 46]. Consistently, inhibition by LRE1 was found to be competitive with bicarbonate but non-competitive with substrate, defining it as the first fully allosteric BBS-targeting sAC inhibitor. LRE1 is a non-toxic, sAC-selective inhibitor that prevented sAC functions in sperm. Importantly, the apo- and ligand-bound sAC structures provide unique insights into the precise mode of binding and key contacts between LRE1 and sAC [23, 46].

### Strategy for Refining the Existing LRE1 Scaffold

Described herein is the identification of sAC inhibitors that balance several important factors. Structural biology data, married to computational support, allowed medicinal chemists to iteratively design and dock potential new ligands into the BBS prior to their synthesis. Moreover, ligand optimization advances by subsequently engineering in enhanced “drug-like” properties to permit their ready absorption as orally dosed agents, minimize metabolic and half-life issues, and build in target specificity while reducing activities at undesired receptors. Synthesized potential inhibitors were tested on human sAC protein in vitro cyclase assays to determine their potency. This iterative design/synthesize/test process greatly improves the ligand optimization process to identify ligands with appropriate intrinsic potency for the sAC binding site.

sAC inhibitors with improved potencies are tested in ancillary assays for safety and useful drug-like qualities, i.e., absorption, distribution, metabolism, excretion, and toxicity (ADME-Tox) studies, and pharmacokinetics (PK). Inhibitors are also screened against tmACs for selectivity, and membrane permeability and ‘in-cell’ efficacy are assessed via assays in sAC-overexpressing cells and sperm. Inhibitors with desired ADME-Tox and PK properties are then injected into animals to test for blockage of sperm capacitation and fertilization with the ultimate goal of identifying a sAC inhibitor that provides on-demand contraception within hours of a single injection and persists long enough to prevent fertilization.

Considerable improvements in the sAC inhibitor scaffolds have been made. Novel sAC inhibitors with improved potency and drug-like characteristics have been developed, providing orally available, on-demand, non-hormonal contraception for men.

### Summary of Contraceptive Uses

A sAC inhibitor can provide on demand, reversible, non-hormonal, oral contraception for hours in men and/or topical or oral contraception for females.

Oral sAC inhibitors provide effective contraception for hours in men. Desirable PK properties for sAC inhibitor male pill include being orally bioavailable and having a quick onset. Additionally, a compound with the appropriate half-life could be used to provide the flexibility to balance efficacy with safety.

Because sAC activity is required in sperm throughout their transit through the female reproductive tract, sAC inhibitors may be useful in females as a non-hormonal, topical inhibitor delivered via an intravaginal ring. The non-hormonal female inhibitor could be desirable. For instance, a sAC inhibitor contraceptive supplied acutely (i.e., as a ring which would be inserted prior to intercourse) to provide transient contraception would be effective for hours to a day. Additionally, sAC contraceptive inhibitors showing low systemic exposure and supplied chronically could be effective for weeks to months. The ring could also be commercialized with sAC contraceptive inhibitor in concert with an anti-STD therapeutic as an MPT (multi-purpose protection technology).

sAC inhibitors can also be used as female oral contraceptives. For example, the compound can be administered either before intercourse or after intercourse to prevent fertilization of an egg. If taken by a female before intercourse or within a period of time after intercourse (e.g., within minutes or hours), an orally-delivered sAC inhibitor can be effective in blocking ejaculated sperm from reaching and fertilizing an egg in the reproductive tract of the female.

### sAC Inhibition by Example 1 Blocks the Bicarbonate-Induced Increase in Beat Frequency in Mouse and Human Sperm

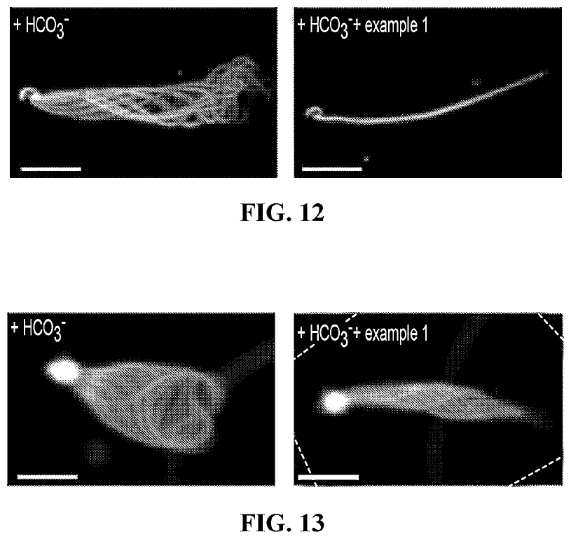

Activation of sAC by bicarbonate not only leads to a rapid increase in intracellular cAMP levels, it also results in an immediate increase in flagellar beat frequency. sAC KO sperm lost the ability to change their flagellar beat frequency after stimulation by bicarbonate and additionally, their flagellar movement is severely impaired. Incubation of WT mouse sperm in the presence of Example 1 mirrored this effect (FIG. 12). Human sperm also increase their beat frequency when incubated in the presence of bicarbonate, and this response was similarly blocked by Example 1 (FIG. 13).

Sperm preparation: Samples of human semen were purified by “swim-up” procedure in human tubular fluid (HTF) (in mM: 97.8 NaCl, 4.69 KCl, 0.2 MgSO₄, 0.37 KH₂PO₄, 2.04 CaCl₂, 0.33 Na-pyruvate, 21.4 lactic acid, 2.78 glucose, 21 HEPES, pH 7.4 adjusted at 37° C. with NaOH). 0.5 to 1 ml of liquefied semen was layered in a 50 ml falcon tube below 7 ml HTF. The tubes were incubated in a tilted angle of 45 degree at 37° C. and 15% CO₂ for 60. Motile sperm were allowed to swim up into the HTF layer, while immotile sperm, as well as other cells or tissue debris, did remain in the ejaculate fraction.

An inverted dark-field video microscope (IX73; Olympus) with a 10× objective (mouse sperm) and a 20× objective (human sperm) (UPLSAPO, NA 0.8; Olympus) was combined with a high-speed camera (ORCA Fusion; Hamamatsu). Dark-field videos were recorded with a frame rate of 200 Hz. The temperature of the heated stage was set to 37° C. (stage top incubator WSKMX; TOKAI HIT).

### Example 1 Blocks In Vitro Fertilization

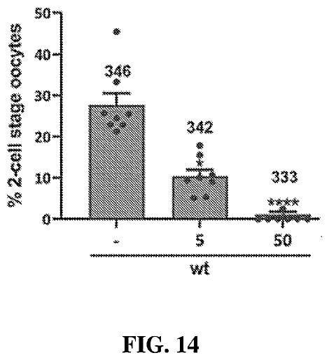

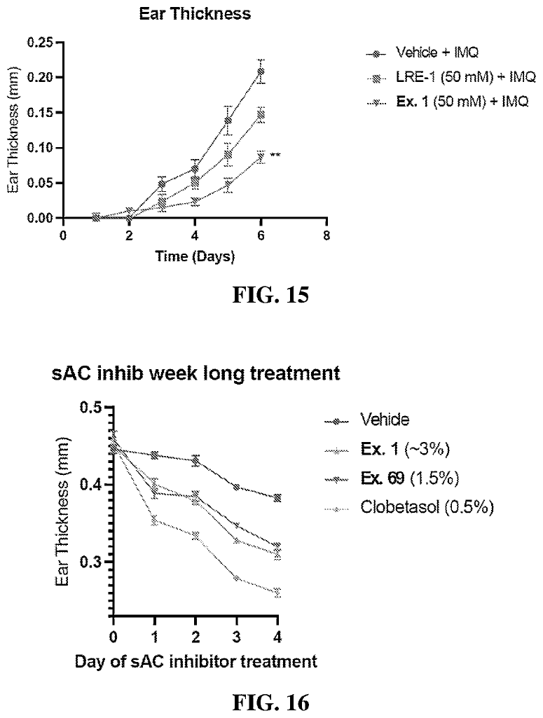

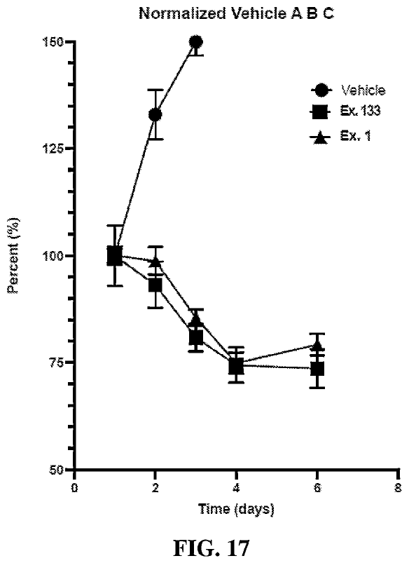

Sperm from the mouse strain C57B1/6 used in this study are inefficient fertilizers in comparison to sperm from other mouse strains, resulting in fertilization rates of 30% in the control. Five (5) M Example 1 reduced the amount of 2-cell stage oocytes to 10%, while 50 μM Example 1 fully blocked fertilization in vitro (FIG. 14).

In vitro fertilization: On the day of preparation, sperm were capacitated for 90 min in HTF medium (EmbryoMax Human Tubal Fluid; Merck Millipore). 100 μl drops of HTF were covered with medium/oil mixture (HTF mixed 1:1 with mineral oil), and 10⁵ sperm were added to each drop. Cumulus-enclosed oocytes were prepared from the oviducts of superovulated females and added to the drops. After 4 hr at 37° C. and 5% CO₂, oocytes were transferred to fresh HTF. The number of 2-cell stages was evaluated after 24 hr.

Male Contraception Via Acute Systemic Inhibition of Soluble Adenylyl Cyclase (sAC)

As described above, with nearly half of all pregnancies unintended, existing family planning options are inadequate. At present, family planning is largely the responsibility of the woman. To achieve reproductive equality, men need more than the two available choices: i.e., condoms or surgical vasectomy. In some embodiments, a novel, acute contraceptive strategy for men is described, which rapidly and temporarily inactivates sperm, that can thereby provide effective on-demand contraception while avoiding the consequences of chronic dosing.

Upon ejaculation, stimulation of bicarbonate-regulated soluble adenylyl cyclase (sAC; ADCY10) is the initial signaling event in sperm. sAC-generated cAMP is essential for sperm motility and capacitation, which are prerequisites for sperm to attain fertilizing capacity (reviewed in References 26, 27, 31). sAC knockout (sAC KO) mice exhibit male-specific sterility [References 35, 37, 61], and two otherwise healthy men homozygous for mutations in the sAC gene (adcy10−/−) are sterile [Reference 39]. Thus, sAC's role as a target for male contraception is genetically validated in mice and men. Besides male-specific fertility, both sAC KO mice and adcy10−/− men exhibit few other phenotypes. sAC KO mice exhibit elevated intraocular pressure [Reference 8], which may predispose them to glaucoma, but this would only develop over long periods of time. And while adcy10−/− men display an increased propensity to form kidney stones [Reference 39], stones can only form during prolonged periods of sAC absence. These sAC null phenotypes suggest a strategy for safe and effective contraception for men by delivering inhibitors which act acutely so that sAC is only transiently blocked [Reference 62].

In mammals, there are two families of adenylyl cyclases which produce the ubiquitously used second messenger cAMP: sAC and G protein regulated, transmembrane adenylyl cyclases (tmACs). Numerous small molecule inhibitors were identified which could selectively target sAC versus tmACs [References 41, 44], and these inhibitors blocked sAC-dependent functions in mouse sperm essential for fertilization in vitro [References 37, 46]. Recently, structure-assisted drug design was used to develop prototypes of increasingly potent sAC inhibitors with drug-like properties suitable for use in vivo to interrogate functions of sAC in animal models [Reference 63]. These inhibitors were used in vitro to validate that sAC inhibitors could be delivered intravaginally as a novel strategy for non-hormonal contraception in women [Reference 62]. In some embodiments herein, the use of acutely acting sAC-specific inhibitors to prove-the-principle that orally available sAC inhibitors can be non-hormonal, on-demand, male contraceptives is described.

A sAC-Specific Inhibitor with High Potency and Long Off-Rate

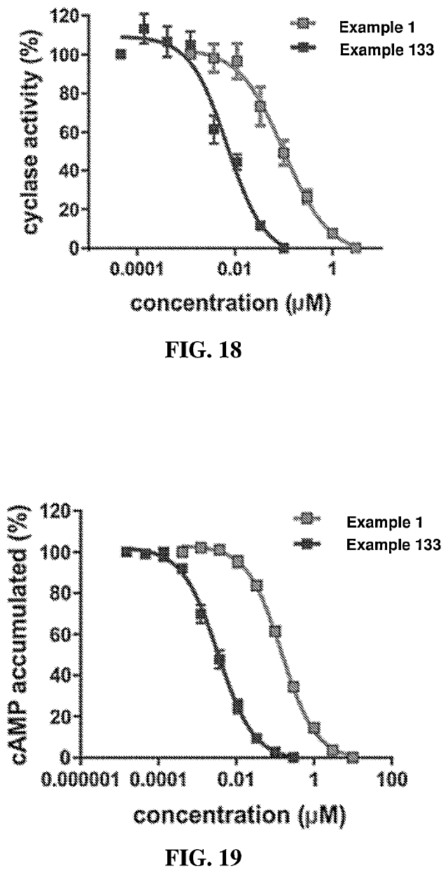

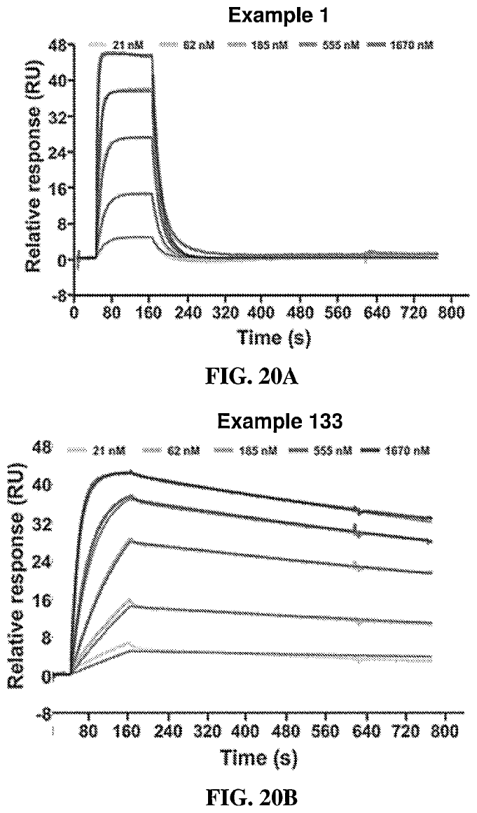

In vitro studies used Example 1 [described above and in Reference 62], a safe and drug-like sAC inhibitor which inhibits sAC with an IC50 of 159 nM. Because its pharmacokinetic profile revealed that intraperitoneal (i.p.) or oral delivery can lead to efficacious levels for hours after a single dose [Reference 63], Example 1 was used in a timed mating study, where Example 1-injected males were paired with receptive females from one hour past injection through 9 hours post injection. Example 1 reduced fertility relative to vehicle-injected males by 25% (Table 16). A sAC inhibitor delivered to the male must retain efficacy post-ejaculation, after sperm containing inhibitor are deposited into the inhibitor-free environment of the female reproductive tract. It is possible that the modest contraceptive efficacy of Example 1 could be due its rapid off-rate from sAC protein [Reference 62], which would mean it was lost from sperm after deposition in the female allowing sAC to become uninhibited post-ejaculation. To confirm the possibilty that inhibitor residency time could be an additional efficacy determining feature, a more potent sAC inhibitor (Example 133) which exhibits long residence time on sAC protein was used (FIG. 20B). As described, Example 1 inhibits purified human sAC protein with an IC₅₀ of 159 nM [Reference 62], while Example 133 inhibited sAC with an IC₅₀ of 3 nM (FIG. 18). To assess potency in a cellular system, 4-4 cells, which stably overexpress sAC, were utilized [References 25, 41]. Cellular levels of cAMP reflected a balance between its synthesis by adenylyl cyclases and its catabolism by phosphodiesterases (PDEs). Hence, in the presence of the non-selective PDE inhibitor IBMX, cells accumulated cAMP solely dependent upon the activity of endogenous adenylyl cyclases, which in 4-4 cells, was exclusively due to sAC [References 25, 41]. As expected, Example 133 (IC₅₀=7 nM) inhibited cAMP accumulation in 4-4 cells with improved potency relative to Example 1 (IC₅₀=102 nM) (FIG. 20B). To compare the binding kinetics (i.e., rate constants for ligand association (k₍ₒₙ₎) and dissociation (k_{(off)}) of Example 1 with Example 133, Surface Plasmon Resonance (SPR) was used, where inhibitor solutions are flowed over a chip containing immobilized recombinant sAC protein. While their kₒₙ are similarly fast, SPR revealed an off-rate (T_{1/2}) for Example 1 (T_{1/2}) of 20 seconds (FIG. 20A), while Example 133 displayed a significantly slower T_{1/2} of 75.8 minutes (FIG. 20B). Thus, in addition to being ˜50 times more potent than Example 1 in in vitro assays and ˜15 times more potent in cellular assays, Example 133 had the benefit of nearly 200 fold longer residence time on sAC protein. Therefore, Example 133 represented a suitable tool compound to determine if residency time on sAC protein was a contraceptive efficacy determining factor necessary to counteract dilution in the female inhibitor-free vagina.

TABLE 16
sAC inhibitors block fertility of male mice in timed matings

| Mating | vehicle | | Example 1 | | | Example 133 | | |
|---|---|---|---|---|---|---|---|---|
| time | Pregnancy | Pregnancy | Pregnancy | Pregnancy | Contraceptive | Pregnancy | Pregnancy | Contraceptive |
| (hours) | rate | pairings | rate | pairings | efficacy | rate | pairings | efficacy |
| 11 | 44.4% | (20/45) | n.d. | | | 15.6% | (7/45) | 65% |
| 8 | 41.1% | (39/95) | 31.1% | (14/45) | 25% | 7.5% | (3/40) | 82% |
| 5 | 31.8% | (27/85) | n.d. | | | 3.2% | (3/92) | 90% |
| 4 days | | | n.d. | | | 92.9% | (13/14) | |
| (1 week | | | | | | | | |
| post- | | | | | | | | |
| injection) | | | | | | | | |

Pregnancies in %, number of pregnancies per total amount of pairings and contraceptive efficacy in % compared to vehicle-injected control of matings using sAC inhibitor-injected males. Vehicle-, 50 mg/kg Example 1- or 50 mg/kg Example 133-injected males were mated with sexually receptive non-injected females for the indicated time periods. One week after Example 133 injection, 14 randomly chosen males were mated with females for four days, showing that contraceptive effect was reversible.

### sAC Inhibitors Prevent Essential Functions In Vitro

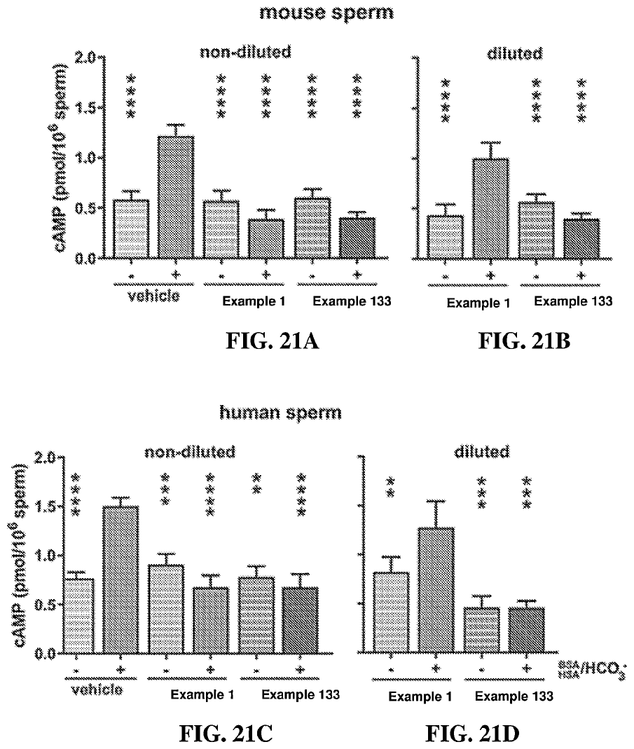

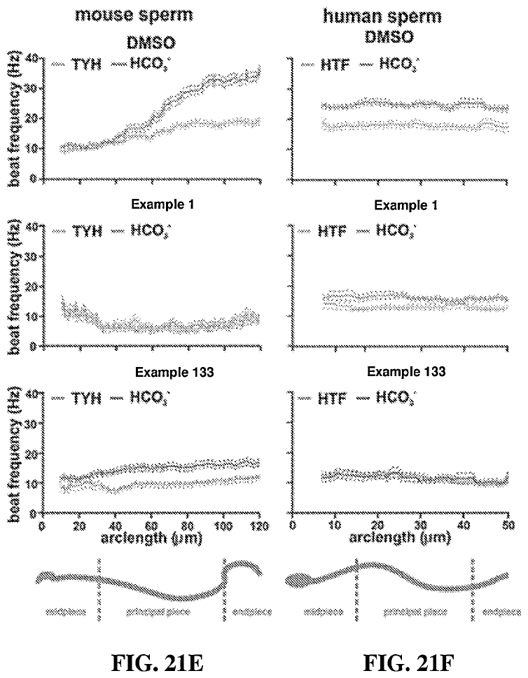

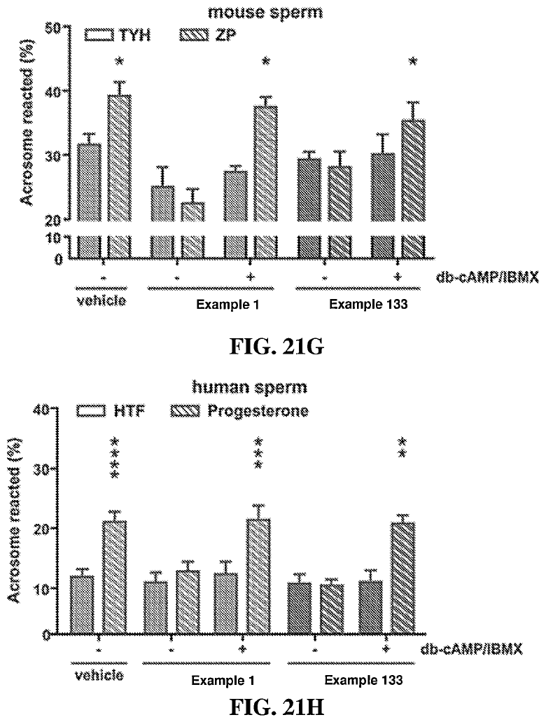

Mammalian sperm are stored in a dormant state within the cauda epididymis where the bicarbonate concentration is actively maintained at ≤5 mM. Upon ejaculation, mixing with seminal fluid exposes the sperm to higher bicarbonate levels (˜25 mM) [References 64, 65], which initiates capacitation via sAC-dependent increase of cAMP. As shown [Reference 62], incubating sperm with 5 μM Example 1 blocks the bicarbonate-induced cAMP rise in mouse and human sperm in vitro. Due to its greater potency, 10 nM Example 133 was sufficient to completely block this response (FIGS. 21A, 21E), and consistent with Example 133's longer residence time on sAC protein (FIGS. 20A, 20B), the ability to inhibit bicarbonate-induced cAMP synthesis survived a 100-fold dilution into inhibitor-free media for Example 133, but not for Example 1 (FIGS. 21B, 21D). Subsequent to the elevation of cAMP, two functional hallmarks of mammalian capacitation are increased flagellar beat frequency, and the ability to undergo a physiologically induced acrosome reaction. Due to its in vitro and cellular potency, Example 133 was also more potent than Example 1 at blocking the bicarbonate-induced increase in flagellar beat frequency (FIGS. 21E, 21F) and acrosome reaction induced by zona pellucidae (in mouse sperm) or progesterone (in human sperm) (FIGS. 21G, 21H). As shown [Reference 62], sAC inhibitors were not toxic to sperm; addition of exogenous cell-permeable cAMP/IBMX rescued the acrosome response blocked by sAC inhibition (FIGS. 21G, 21H). Thus, Example 133 was more potent and displayed longer residence times than Example 1 when treating sperm in vitro.

### Systemic Delivery of sAC Inhibitors Block Sperm Functions Ex Vivo

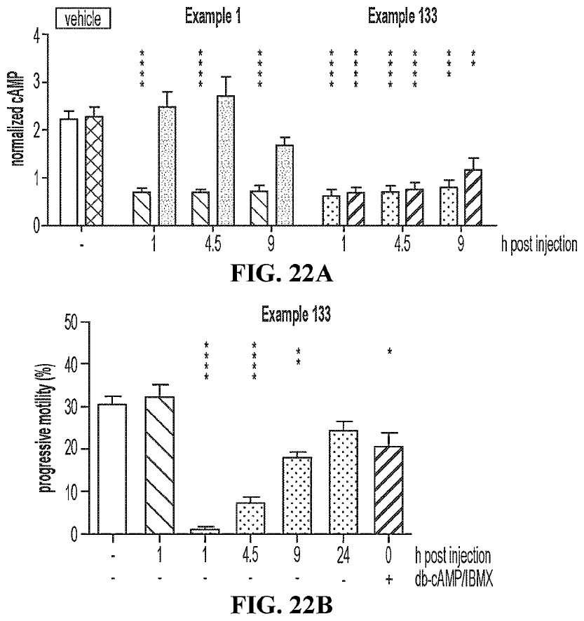

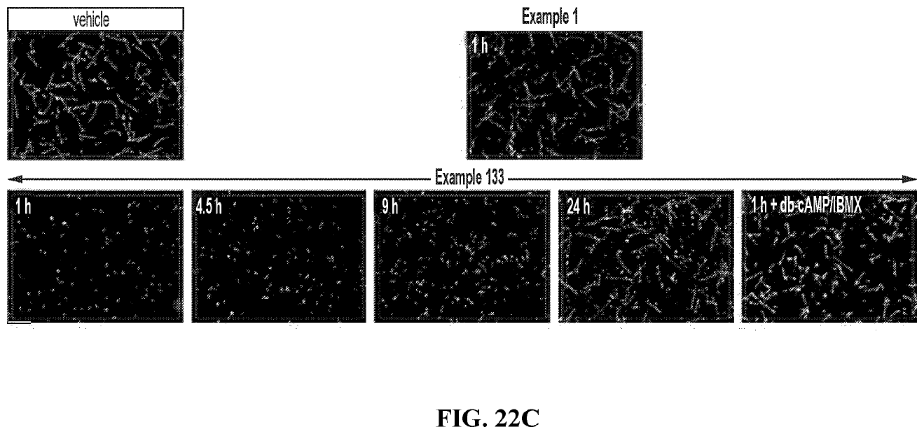

Similar to Example 1, Example 133 reaches maximum serum levels (C max) within 7.5 minutes following a single intraperitoneal (i.p.) injection. Therefore, both inhibitors were suitable to determine whether systemic delivery of sAC inhibitors could inhibit sperm functions isolated from inhibitor injected male mice (i.e., ex vivo). In sperm isolated from vehicle-injected mice, bicarbonate induced a ˜3 fold increase in cAMP (FIG. 22A). When assessed in sperm that were minimally diluted following isolation, the bicarbonate-induced cAMP increase was absent in sperm from Example 1 or Example 133 injected mice isolated one hour after i.p. injection. Inhibition of bicarbonate-induced cAMP persisted for 4.5 hours and 9 hours post-injection in minimally diluted sperm isolated from mice injected with either inhibitor. The difference in off-rates between the two inhibitors (FIGS. 20A, 20B) was evident when sperm from injected mice were diluted 1:200 ex vivo. Dilution restored bicarbonate-responsive cAMP synthesis in sperm isolated from mice injected with the fast off-rate inhibitor Example 1 (FIG. 22A). In contrast, in mice injected with the slow off-rate inhibitor Example 133, the cAMP response in sperm isolated 1 hour or 4.5 hours post-injection remained inhibited following dilution ex vivo. By 9 hours post-injection with Example 133, cAMP responsiveness partially recovered with dilution.

Fertility is dependent upon sperm progressive motility [Reference 66]. sAC KO mice and humans with sAC mutations are male-specific infertile, and their sperm show only small vibratory movements [References 35, 37-39, 62]. To microscopically assess motility, sperm need to be diluted to the same degree as the ‘diluted’ conditions (i.e., 1:200) in the ex vivo cAMP measurements (FIG. 22A). Sperm from mice isolated one hour post-injection with Example 1 were indistinguishable from sperm isolated from vehicle-injected mice (FIGS. 22B, 25), consistent with the injected Example 1 not surviving substantial ex vivo dilution in the cAMP assay. In contrast, sperm isolated from mice one hour after injection with Example 133 were essentially immotile (FIGS. 22B, 25), displaying only vibratory movement reminiscent of sperm from sAC KO mice [References 35, 37, 61, 62] and humans [Reference 39]. By 4.5 hours post injection, a subset of the sperm (8%) from Example 133 injected mice recovered motility, and an even greater percentage of sperm recovered motility (20%) by 9 hours post injection (FIG. 22B). The addition of exogenous membrane-permeable cAMP rescued motility in Example 133 injected sperm, which confirmed that Example 133 was not cytotoxic and functioned via inhibiting sAC.

### Systemic Delivery of a Single Dose of Example 133 Inhibits Fertility In Vivo

Because Example 133 was more potent (FIGS. 18, 19) and had longer residence times than Example 1 (FIGS. 20A, 20B), and was suitable for interrogation of sAC functions in vivo (FIGS. 22A-22B), timed mating studies were employed to assess its contraceptive efficacy. One hour after male mice were injected with vehicle control or Example 133, they were paired with receptive females (i.e., females visually identified to be in estrus) for the subsequent 10 hours (until hour 11 post injection), 8 hours (until hour 9 post injection), or 5 hours (until hour 6 post injection). Pairing vehicle-injected males with receptive females for 11, 8, or 5 hours resulted in 44%, 41%, and 32% pregnancy rates, respectively (Table 16). Injection with Example 133 did not adversely affect mouse behavior; movements, and mating behaviors were indistinguishable between Example 133 or vehicle-injected males, and similar numbers of mating plugs were observed following vehicle and inhibitor injected pairings. When Example 133 injected males were paired with receptive females from 1 hour post injection to 6 hours post injection (5 hour pairing), the pregnancy rate was reduced to 3.3%, corresponding to 90% contraceptive efficacy. During this mating window, Example 133 injected mice were beginning to show restored functions; approximately 8% of their sperm were motile at 4.5 hours post injection (FIG. 22B). By 9 hours after mice were injected with Example 133, their sperm displayed improved functionality; they partially recovered bicarbonate-induced cAMP response following dilution (FIG. 22A) and ˜20% of the sperm displayed progressive motility (FIG. 22B). When Example 133 injected males were paired with receptive females from 1 hour post injection to 9 hours post injection (8 hour pairing) or even longer, to 12 hours post injection (11 hour pairing), the pregnancy rates improved from the 5 hour pairing, but they were still significantly reduced relative to vehicle control. For 8-hour pairings, the pregnancy rate was 7.2%, corresponding to 82% contraceptive efficacy, and for 11 hour pairings, the pregnancy rate was 16%, corresponding to 65% contraceptive efficacy. The 82% contraceptive efficacy observed in mice injected with the long off-rate inhibitor Example 133 during an 8 hour pairing represents a significant improvement from pairings using mice injected with the fast off-rate inhibitor Example 1, which showed 25% contraceptive efficacy over the same period. Thus, this confirmed that contraceptive efficacy would improve with slow off-rate inhibitors; compounds with slow off-rate could withstand the inevitable dilution in the inhibitor-free female following ejaculation improving their contraceptive efficacy. In summary, maximal contraceptive efficacy was achieved over the shortest mating window attempted (i.e., 5 hours), and efficacy of the slow off rate sAC inhibitor Example 133 far exceeded efficacy with a fast-off rate inhibitor (Example 1). On-demand contraception via sAC inhibition is aided by both pharmacokinetic and binding kinetic properties.

Among the instances (from each of the timed mating windows) where inhibitor-injected males impregnated females, pregnancies were normal. Litter sizes were indistinguishable between sAC-inhibitor injected males compared to those from vehicle-injected males, and both male and female F1 progeny from breakthrough pregnancies matured normally into fertile adults. Furthermore, no evidence was identified that reduced pregnancies from sAC inhibitor injected males were due to abortifacient activity. When pregnancies were assessed 7 days post mating by uterine inspection, there were no signs of aborted fetuses in females mated with either sAC-inhibitor- or vehicle-treated males. Finally, effects on fertility were fully reversible. One week after injection, randomly chosen Example 133 injected males were mated with females. To maximize mating efficiency males were paired with females for four days, and 93% of the pairings yielded litters.

### sAC Inhibitors Block Hyperactivation in Human Sperm

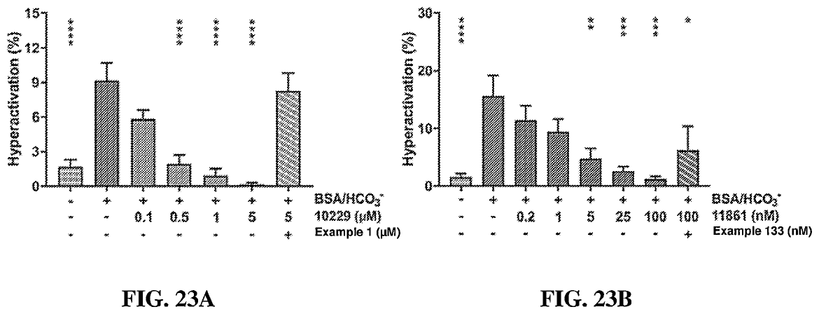

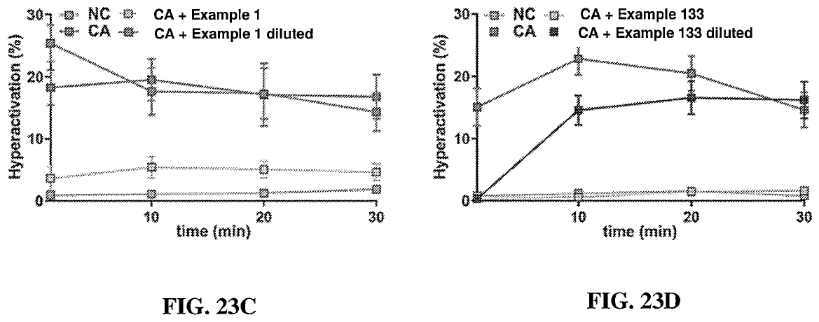

A major distinction between mouse and human reproduction is the female anatomy. In mice, there is no physical barrier between vagina and uterus, and semen is deposited directly into the uterus [Reference 67]. In humans, ejaculated sperm must cross the cervix to escape the normally inhospitable environment of the vagina to enter the permissive environment of the uterus. Once sperm cross the cervix, they can persist for days allowing human conception to occur days following copulation [Reference 68]. During capacitation, human sperm alter their motility to a vigorous, asynchronous beating pattern known as hyperactivation, and it is this altered motility pattern which facilitates human sperm crossing the cervical mucus barrier [References 69, 70]. Human sperm achieve their maximum level of hyperactivation at the earliest time points measured (FIG. 23A), indicating it is an early event during capacitation consistent with it being required for human sperm to cross the cervix to escape the vagina. Both Example 1 and Example 133 blocked hyperactivation of human sperm in vitro (FIGS. 23A, 23B), verifying human sperm hyperactivation is dependent upon sAC. Consistent with the differences in their off-rates, Example 1 inhibition was lost following dilution into inhibitor-free media (FIG. 23E), while Example 133 inhibition persisted following dilution (FIG. 23D). Thus, it is possible that sperm from a man who has taken a slow off-rate sAC inhibitor contraceptive will be immotile (FIG. 22B) and/or fail to hyperactivate even after sperm are ejaculated into the inhibitor-free vagina. Such long-residence time inhibited sperm would be trapped in the acidifying vaginal compartment, which is inhospitable to sperm. Once the vagina re-acidifies following intercourse [Reference 71], the sAC-inhibited, trapped sperm will inactivate and be unable to continue their journey through the female reproductive tract. In some embodiments a framework for developing an on-demand male contraceptive is shown. In addition to the usual efficacy determining factors, e.g., potency, selectivity, and pharmacokinetics, slow off-rate was identified as a feature potentially important to on-demand male contraceptives. Long residence time on the sperm target helps counter the inevitable dilution in the female following ejaculation.

The on-demand strategy described in some embodiments is qualitatively distinct from other efforts to develop a male contraceptive. Hormonal strategies, which block sperm production, are in clinical trials. They require months of continuous usage before sperm numbers fall to subfertile levels and need months after cessation of therapy for recovery of normal sperm counts [References 72-75]. There are other strategies validated in animal models which do not depend upon disrupting sperm production. Unlike these methods, on-demand contraception with a sAC inhibitor protected male mice within an hour, and fertility was fully restored days later.

Besides being more convenient, therapeutics acting acutely are less likely to elicit unwanted side effects than chronic treatments. Specifically for sAC, besides male-specific infertility, the phenotypes observed in mice or men in the absence of sAC need long periods of time to manifest. Elevated intraocular pressure requires years to cause glaucoma [Reference 43], and kidney stones will only form after prolonged absence of sAC. Plus, there is precedent for an on-demand therapeutic targeting a broadly expressed target being safely administered and widely adopted. Like sAC, the target of erectile dysfunction therapeutics, the cGMP-specific phosphodiesterase 5 (PDE5), is widely expressed [Reference 57], yet acute PDE5 inhibitors (i.e., sildenafil, vardenafil, tadalafil) are sufficiently safe for widespread use [Reference 79].

The data and studies presented herein demonstrate the principle that on-demand contraception in men is possible. A sAC inhibitor with suitable pharmacokinetics, long residence time, and safety profile can be formulated into an oral male birth control pill, which a man would take half an hour to an hour before sex, and he would be protected from unwanted pregnancy for the following hours. This innovative on-demand strategy represents a new paradigm in contraception, which like the advent of oral birth control for women, has the potential to revolutionize family planning.

### Methods

### Reagents, Cell Lines, and Mice

3-Isobutyl-1-methylxanthine (IBM X), BSA, dibutyryl-cAMP (db-cAMP), hyaluronidase, lectin from Pisum sativum FITC-conjugated (PSA-FITC) and lectin from Arachis hypogaea FITC-conjugated (PNA-FITC) were purchased from Sigma-Aldrich, ionomycin from Tocris, β-mercaptoethanol from Gibco, and hormones from ProSpec. PBS buffer was purchased from Corning, DMEM and 0.5 M EDTA, pH 8.0 from Thermo Fisher Scientific, FBS from Avantor Seradigm and polyethylene glycol 400 (PEG 400) from Merck Millipore.

sAC-overexpressing 4-4 cells were generated and functionally authenticated in our laboratory as previously described [Reference 41] and grown in DMEM+10% FBS. Cells were maintained at 37° C. in 5% CO₂ and were periodically checked for mycoplasma contamination.

Adult C57BL/6J male and female mice and CD1 mice were purchased and allowed to acclimatize before use. Animal experiments were approved by Weill Cornell Medicine's Institutional Animal Care and Use Committee (IACUC).

### Sperm Isolation

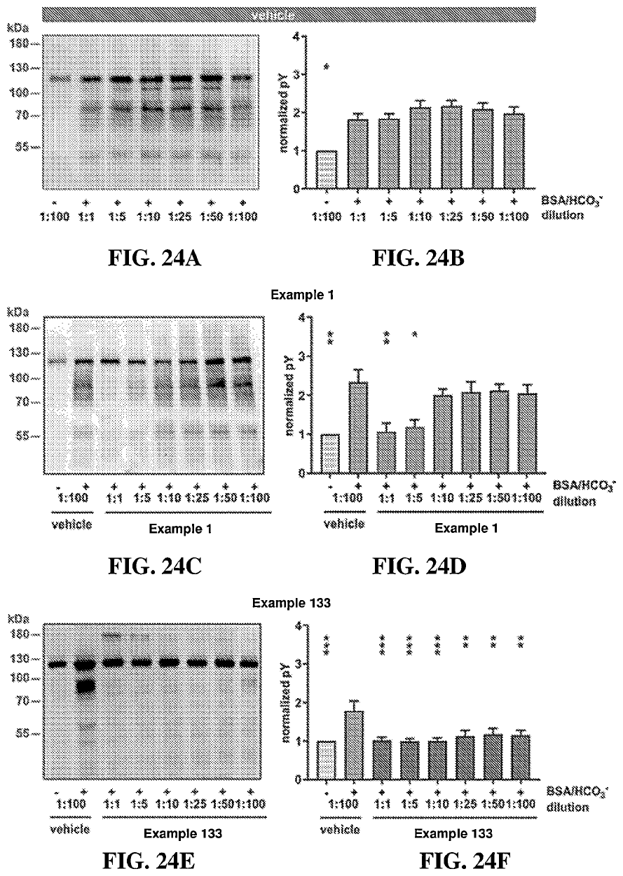

Mouse sperm were isolated by incision of the cauda epididymis followed by ‘swim-out’ in 500 μl Toyoda Yokoyama Hoshi (TYH) medium (in mM: 135 NaCl, 4.7 KCl, 1.7 CaCl₂, 1.2 KH₂PO₄, 1.2 MgSO₄, 5.6 glucose, 0.56 pyruvate, 10 HEPES, pH 7.4 adjusted at 37° C. with NaOH), prewarmed to 37° C. After 15 minutes swim-out at 37° C., sperm from two caudae were combined and counted using a hematocytometer. For capacitation, sperm were incubated for 90 minutes in TYH containing 3 mg/ml BSA and 25 mM NaHCO₃ in a 37° C. incubator. To control for the consequences of dilution during isolation of epididyrnal mouse sperm for ex vivo assays, the ability for bicarbonate to induce a prototypical pattern of tyrosine phosphorylation (pY) which is a widely used molecular hallmark of capacitation was assessed [Reference 80]. Bicarbonate-induced pY is known to be sAC dependent in vitro [References 37, 46, 62]. For experiments studying sperm from injected mice, ‘swim out’ was performed in in 200 μl TYH, which corresponded to a 1:10 dilution from epididymis (20 μg cauda in 200 μl buffer). Capacitation induced changes were assessed by adding 50 μl of ‘swim out’ sperm to increasing volumes of non-capacitating or capacitating TYH buffer. Sperm from vehicle-injected mice showed the capacitation-induced increase in pY regardless of dilution in capacitation media (FIGS. 24A, 24B). The pY pattern was blocked in sperm from both Example 1 and Example 133 injected mice when the ‘swim out’ sperm were minimally diluted by mixing with equal volume capacitation media (FIGS. 24E, 24F). When ‘diluted 25 fold, the pY pattern was restored in sperm from the fast off-rate inhibitor, Example 1 (FIGS. 24C, 24D). In contrast, the pY pattern remained blocked in ‘swim out’ sperm from Example 133 injected mice even when they were diluted 100 fold (FIGS. 24E, 24F). Because Example 1 inhibition survived the minimal dilution, but not the more substantial (i.e., 25 fold) dilution, ex vivo bicarbonate-induced cAMP changes were compared under these different conditions.

Samples of human semen were obtained from healthy volunteers with their prior written consent. Only samples that met the WHO 2010 criteria for normal semen parameters (ejaculated volume ≥1.5 mL, sperm concentration ≥15 million/mL, motility ≥40%, progressive motility 32%, normal morphology ≥4%) were included. Semen was incubated for 30 minutes in a 37° C. incubator to liquefy. Human sperm were purified by “swim-up” procedure in human tubular fluid (HTF) (in mM: 97.8 NaCl, 4.69 KCl, 0.2 MgSO₄, 0.37 KH₂PO₄, 2.04 CaCl₂, 0.33 Na-pyruvate, 2.78 glucose, 21 HEPES, pH 7.4 adjusted at 37° C. with NaOH). 0.5 to 1 ml of liquefied semen was layered in a 50 ml tube below 4 ml HTF. The tubes were incubated at a tilted angle of 450 at 37° C. for 60 minutes. Motile sperm were allowed to swim up into the HTF layer; immotile sperm and other cells or tissue debris remain in the ejaculate fraction. Up to 3 ml of the HTF layer was transferred to a fresh tube and washed twice in HTF by centrifugation (700×g, 20 minutes). For CASA experiments, human sperm were purified by density gradient centrifugation using Isolate (Irvine Scientific). 1 ml of sperm were layered on top of 2 ml of the upper layer (50%) and 2 ml of the lower layer (90%) and centrifuged at 300×g for 20 minutes. The supernatant was removed, the remaining 0.5 ml sperm layer was resuspended in 3 ml non-capacitating HTF buffer and centrifuged at 300×g for 10 minutes. For both purification methods, the supernatant was removed after the last centrifugation step and the sperm pellet was resuspended in 1 ml HTF. The purity and vitality of each sample was assessed via light microscopy. Sperm cell numbers were determined using a hemocytometer and adjusted to a concentration of 1×10⁷ cells/ml. For capacitation, sperm were incubated in HTF with 72.8 mM NaCl containing 25 mM NaHCO₃ and 3 mg/ml human serum albumin (HSA) (Irvine Scientific, Santa Ana, CA, USA) or 3 mg/ml BSA for up to 3 hours.

### In Vitro Adenylyl Cyclase Activity Assay

All in vitro adenylyl cyclase activity assays were performed via the “two-column” method measuring the conversion of [α-³²P] ATP into [³²P] cAMP, as previously described [References 81, 82]. Briefly, human sACₜ protein [Reference 21] was incubated in buffer containing 50 mM Tris-HCl, pH 7.5, 4 mM MgCl₂, 2 mM CaCl₂, 1 mM ATP, 3 mM DTT, 40 mM NaHCO₃ in the presence of the indicated concentrations of different sAC inhibitors or vehicle (DMSO).

Cellular cAMP Accumulation

sAC-dependent cAMP accumulation was measured in sACᵣ-overexpressing 4-4 cells. On the day prior to the assay, 5×10⁶ cells/ml were seeded in 24-well plates in DMEM with 10% FBS. To measure sAC-dependent cAMP accumulation, cells were pretreated for 10 minutes with the respective inhibitor at the indicated concentrations or DMSO as control in 300 μl fresh media. Cyclic AMP accumulation was initiated by the addition of 500 μM IBMX, and after 5 minutes, the media was removed and the cells were lysed with 250 μl 0.1 M HCl by shaking at 700 rpm for 10 min. Cell lysates were centrifuged at 2000×g for 3 minutes and the cAMP in the supernatant was quantified using the Direct cAMP ELISA (Enzo) according to the manufacturer's instructions.

cAMP generation was measured in mouse and human sperm. For mouse sperm, aliquots of 2×10⁶ mouse sperm were incubated for 12 minutes in the presence or absence of sAC inhibitor in non-capacitating or capacitating TYH buffer. For human sperm, aliquots of 2×10⁶ human sperm were incubated for 30 minutes in the presence or absence of sAC inhibitor in non-capacitating or capacitating HTF buffer. In both cases, 0.1% DMSO was used as vehicle control.

For wash-out experiments assessing dilution of sAC inhibitors from sperm, sperm were pre-incubated for 5 minutes in non-capacitating media in the presence of sAC inhibitor at a concentration 5× above its IC₅₀. After 5 min, 150 μl of sperm/inhibitor mix was diluted into 1.35 ml non-capacitating or capacitating media with no inhibitor. After 12 minutes (mouse sperm) or 30 minutes (human sperm), sperm were sedimented by centrifugation at 2,000×g for 3 minutes and lysed in 200 μl HCl for 10 minutes. Sperm lysates were centrifuged at 2,000×g for 3 minutes and the cAMP in the supernatant was acetylated and quantified using the Direct cAMP ELISA (Enzo).

For ex vivo determination of sperm cAMP generation, male mice were injected intraperitoneally (i.p.) with 150 μl of solution containing sAC inhibitor; control males were injected with 150 μl vehicle control (DMSO:PEG 400 1:4 (v/v) for Example 1, DMSO:PEG 400:PBS 1:4:5 (v/v) for Example 133). Sperm (50 μl) isolated at indicated time points (between 1 hour and 24 hours post-injection) were incubated in 50 ul (1:20 dilution) or 450 μl (1:200 dilution) non-capacitating or capacitating TYH media for 12 minutes. Intracellular cAMP levels were quantified using the Direct cAMP ELISA (Enzo) as above.

### Measuring Binding Kinetics Using Surface Plasmon Resonance

Association and dissociation rate constants of sAC inhibitors were obtained with a Biacore 8K instrument (Cytiva) using a parallel kinetics protocol. Series S Sensor NTA chips (Cytiva) were prepared by applying recombinant purified His-tagged sACₜ protein (50 μg/ml) in PBS-P+ buffer (1 mM KH₂PO₄, 150 mM NaCl, 6 mM Na₂HPO₄, 0.05% (w/v) P20 Surfactant). The His-tagged sAC protein was captured via Ni²⁺-His-tag chelation and covalently immobilized by amine coupling with a 1:1 mixture of 1-ethyl-3-(3-dimethylaminopropyl) carbodiimide and N-hydroxysuccinimide (active channels). After coupling, remaining reactive groups on the chip's surface were blocked with 1 M ethanolamine followed by 350 mM EDTA to wash away any free Ni²⁺. Following preparation, TBS-P+ running buffer (50 mM Tris, 150 mM NaCl, and 0.05% P20 Surfactant) supplemented with 1% DMSO was flowed over the surface of the chip until a stable baseline was obtained. For each sAC inhibitor, increasing concentrations were injected into parallel channels for 120 seconds at a flow rate of 50 μl/minute followed by running buffer for 600 seconds to allow for dissociation. All experiments included reference channels; i.e., inhibitor run over parallel channels without immobilized protein. Binding kinetics were determined by subtracting responses in the reference channels from responses in the active channels. Curves were fitted, and kₒₙ and k_{off} values were determined using the Biacore 8K Insight Evaluation Software Version 2.0 (Cytiva) and a 1:1 binding kinetics model.

### Isolation of Mouse Zone Pellucida

Zonae pellucidae were isolated from female mice superovulated by intraperitoneal injection of 10 I.U. human chorionic gonadotropin 3 days before the experiment. 14 hours before oocyte isolation, mice were injected with 10 I.U. pregnant mare's serum gonadotropin. Oviducts were collected following cervical dislocation. Cumulus-enclosed oocytes were separated from the oviducts and placed into TYH buffer containing 300 μg/ml hyaluronidase. After 15 minutes, cumulus-free oocytes were transferred into fresh buffer and washed twice. Zonae pellucidae and oocytes were separated by shear forces generated by expulsion from 50 nm pasteur pipettes. Zona pellucidae were counted, transferred into fresh buffer and heat-solubilized by incubation for 10 minutes at 65° C.

### Acrosome Reaction Assay

For analysis of acrosomal exocytosis, 100 μl of 1×10⁷ sperm/ml were capacitated for 90 minutes in TYH buffer supplemented with 3 mg/ml BSA and 25 mM NaHCO₃ (mouse sperm) or HTF buffer supplemented with 3 μl/ml HSA and 25 mM NaHCO₃ (human sperm). sAC inhibitors were added with capacitating buffer; 0.1% DMSO was used as vehicle control. Acrosome reaction was induced by incubating mouse sperm with 50 mouse solubilized zonae pellucidae for 15 minutes at 37° C., or human sperm with 10 μM progesterone for 30 minutes at 37° C. The sperm suspensions were sedimented by centrifugation at 2,000×g for 5 minutes and the sedimented sperm were resuspended in 100 μl PBS buffer. Samples were air-dried on microscope slides and fixed for 30 minutes in 100% ethanol at room temperature (RT). For acrosome staining, mouse and human sperm were incubated for 30 minutes in the dark with 5 μg/ml PNA-FITC or 5 μg/ml PSA-FITC, respectively, and counterstained with 2 μg/ml DAPI. After curing, slides were analyzed using a Zeiss LSM 880 Laser Scanning Confocal Microscope; images were captured with two photomultiplier and one Gallium Arsenide Phosphide detector using ZEN Imaging software. For each condition, at least 600 cells were analyzed using ImageJ 1.52.

### Western Blot Analysis

Mouse sperm from vehicle or inhibitor-injected mice were isolated 1 hour from male mice injected (i.p.) with 150 μl of solution containing sAC inhibitor or vehicle. Sperm were diluted 1:20 to 1:1000 in capacitating TYH media. As control, sperm from vehicle-injected mice were diluted 1:1000 in non-capacitating and capacitating TYH buffer. The samples were incubated for 90 minutes, washed with 1 ml PBS and sedimented by centrifugation at 2,000×g for 3 minutes. The sedimented sperm were resuspended in 15 μl 2× Laemmli sample buffer [Reference 83], heated for 5 minutes at 95° C., supplemented with 8 μl β-mercaptoethanol and heated again for 5 minutes at 95° C. For Western blot analysis, proteins were transferred onto PVDF membranes (Thermo Scientific), probed with anti-phosphotyrosine antibodies, and analyzed using a cherniluminescence detection system. Image lab (Bio-Rad) was used for densitometric analysis of Western blots.

### Sperm Motility Assays

For single-sperm motility analysis, mouse and human sperm tethered to a glass surface were observed in shallow perfusion chambers with 200 μm depth. An inverted dark-field video microscope (IX73; Olympus) with a 10×objective (mouse sperm) or a 20×objective (human sperm) (UPLSAPO, NA 0.8; Olympus) was combined with a high-speed camera (ORCA Fusion; Hamamatsu). Dark-field videos were recorded with a frame rate of 200 Hz. The temperature of the heated stage was set to 37° C. (stage top incubator WSKMX; TOKAI HIT). The images were preprocessed with the imageJ plugin SpermQ Preparator (Gaussian blur with sigma 0.5 px; Subtract background method with radius 5 px) and analyzed using the ImageJ plugin SpermQ [Reference 84]. The beat frequency was determined from the highest peak in the frequency spectrum of the curvature time course, obtained by Fast Fourier Transform.

For ex vivo assessment of mouse sperm from inhibitor-injected mice, sperm (25 μl) isolated at the indicated time points (1 hour to 24 hours post-injection) were loaded on a 100 μM Leja slide (Hamliton Thorne) and placed on a microscope stage at 37° C. Sperm movements of 10 fields of at least 500 sperm were examined using computer-assisted sperm analysis (CASA) via Hamilton-Thorne digital image analyzer (IVOS II, Hamilton Thorne Research, Beverly, MA) with the following parameters: 30 frames, frame rate: 60 Hz, cell size: 30-170 μm².

For human sperm, hyperactivation was assessed via CASA following incubation for 1 hour in non-capacitating HTF buffer or capacitating HTF buffer supplemented with 25 mM HCO₃ and 5 mg/ml BSA in the presence or absence of the indicated concentration of sAC inhibitor; 0.1% DMSO was used as vehicle control. For rescue experiments, sperm were incubated in the presence of 5 mM db-cAMP and 500 μM IBMX. 8 μl sperm suspension was placed onto a microscope slide (Gold Seal, Erie Scientific, Portsmouth, NH) and covered with a 18×18 mm coverslip (globe Scientific, Mahway, NJ) to create a 20 μl imaging chamber. CASA was performed following analysis guidelines provided by the company, i.e., 30 frames were acquired at a rate of 60 Hz, at least 200 sperm were analyzed per condition. The following parameters were measured: mean path velocity (VAP, μm/sec), curvilinear velocity (VCL, μm/sec), straight-line velocity (VSL, μm/sec), amplitude of lateral head displacement (ALH, μm), and beat cross frequency (BCF, Hz). Human sperm were considered hyperactivated when presenting VCL ≥150 μm/sec, LIN <50%, and ALH ≥5 m.

To assess motility following ‘washout’ of sAC inhibitors, 5×10⁸ human sperm were preincubated for 20 min with 10 μM Example 1 or 100 nM Example 133 and diluted 1:100 in inhibitor-free or inhibitor-containing media. Motility parameters were determined 1 to 45 minutes post dilution, DMSO-treated non-capacitated and capacitated human sperm were used as control. To calculate the percentage of inhibition, the hyperactivation percentage of sperm diluted in inhibitor-containing- or inhibitor-free media was normalized to the hyperactivation percentage of vehicle-treated capacitated sperm at the respective time point and subtracted from 100%.

### Mouse Mating

Single-housed naïve (i.e., uninjected and virgin) male and female C57B1/6 mice were acclimatized to reverse light cycle (dark: 11 am to 11 pm) for at least two weeks. At 10.00 am, males were injected (i.p.) with 150 μl sAC inhibitor solution or 150 μl vehicle control (DMSO:PEG 400 1:4 (v/v) for Example 1, DMSO:PEG 400:PBS 1:4:5 (v/v) for Example 133). One hour later (11 am), individual injected males were paired with a female in estrus (identified by physical examination within the previous 30 minutes), and the pair was allowed to mate for the subsequent 5, 8, or 11 hours. Pregnancy and litter size were assessed in two ways. Either females were sacrificed 7 days following mating and implanted embryos counted, or females were permitted to go to term (21 days) and pups counted. A subset of the pups (both male and female) born from breakthrough pregnancies were permitted to mature and their fertility assessed in standard matings. To test fertility recovery after Example 133 injection, one week after injection with 50 mg/kg Example 133, individual males were mated for four days with a female and pregnancy (and litter size) assessed after 21 days.

### Statistical Analysis

Statistical analyses were performed using GraphPad Prism 5 (Graph-Pad Software). All data are shown as the mean±SEM. Statistical significance between two groups was determined using two-tailed, unpaired t-tests with Welch correction, and statistical significance between multiple groups using one-way ANOVA with Dunnett correction. Differences were considered to be significant if *P<0.05, **P<0.01, ***P<0.001, and ****P<0.0001.

### Ocular Conditions

### Inhibition of sAC Elevates Intraocular Pressure (IOP)

Ocular hypotony (i.e., idiopathic hypotony) is a very rare orphan disease and no approved or off-label therapies are currently available. sAC inhibitors can be used to treat ocular hypotony by elevating intraocular pressure (IOP). One potential use of sAC inhibitors is to prevent hypotony post glaucoma surgery. Transiently elevating IOP during recovery can permit more aggressive corrective surgeries.

### IOP Experimental Data and Methods

### IOP Measurement

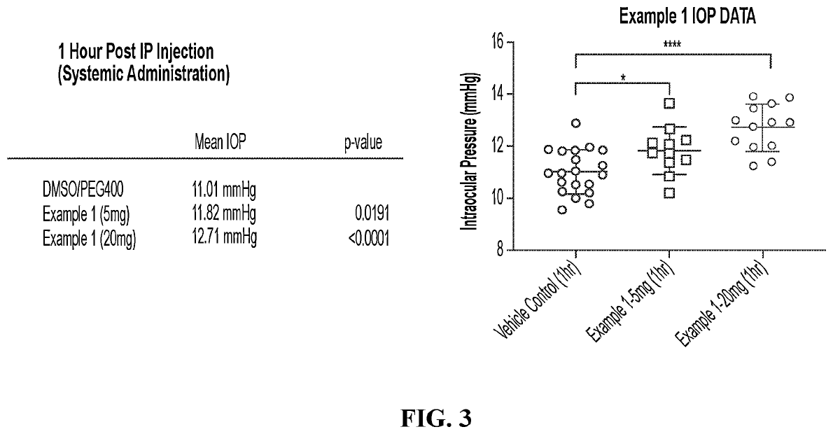

Example 1 elevates IOP in mice as shown in FIG. 3. A mouse was anesthetized via intraperitoneal (ip) injection of room temperature ketamine/xylazine. Anesthesia was assessed by pinching back. The anesthetized mouse was placed on a platform. The anterior chamber was cannulated using a 33-gauge stainless steel needle. The needle was inserted anterior to the limbus and through the cornea. The cannula was connected to a pressure transducer using teflon tubing and was calibrated to a water height equivalent of 0 mm Hg. The transducer signal was amplified, converted to a digital signal, and the voltage was recorded using LabScribe3 software. IOP was calculated by comparing the change in voltage to a standard calibration curve generated at the end of each experiment using the water height column.

### Inflammation and Immune Response

### sAC Inhibition Reduces Type 17 Inflammation

Currently, there are limited non-steroidal topical therapeutics for Th17-mediated skin disease. Cyclic AMP (cAMP) can have both positive and negative effects on T cell biology. The sources of cAMP and mechanisms of cAMP-dependent T cell activation remain poorly understood. sAC, encoded by the ADCY10 gene, is expressed in skin T cells from patients with psoriasis but whether this source of cAMP is important for type 17 inflammation or Th17 cell activation was not clear. It is shown that sAC-dependent cAMP is required for Th17 cell activation and type 17 inflammation in mice. Adcy10^{-/-} mice were unable to mount a normal IL-17-mediated inflammatory response. Stimulation of Adcy10^{-/-} mouse skin with imiquimod led to markedly reduced erythema, scaling and swelling. Adcy10^{-/-} mice following imiquimod treatment had reduced Th17 cell numbers, IL-17 expression, and IL-17-dependent gene expression profile as compared to wild-type mice. Genetic and pharmacologic sAC inhibition inhibited Th17 cell polarization in vitro but had no effect on keratinocyte response to IL-17 and IL-22, suggesting that sAC is, at least, necessary for Th17 cell activation and the effects of sAC loss on type 17 dependent inflammation are due in part to a Th17 cell intrinsic defect. RNAseq analysis of T cells from wild type and Adcy10 mice during polarization towards a Th17 phenotype confirmed that sAC is essential for Th17 cell activation. However, traditional lineage defining Th17 transcription factors, such as RORc, were not affected by sAC. sAC activity is required for CREB-dependent gene expression induced by Th17 polarizing cytokines. Small molecule sAC inhibitors can safely penetrate the skin and can affect cutaneous biology. Similar to genetic inhibition of sAC, topical application of sAC inhibitors (sACi) significantly reduce type 17 inflammation and IL-17 gene expression in the skin. In conclusion, sAC appears to be critical for type 17 inflammation and Th17 cell activation in the skin and sACi may represent a new class of non-steroidal anti-inflammatory therapeutics.

### Experimental Results

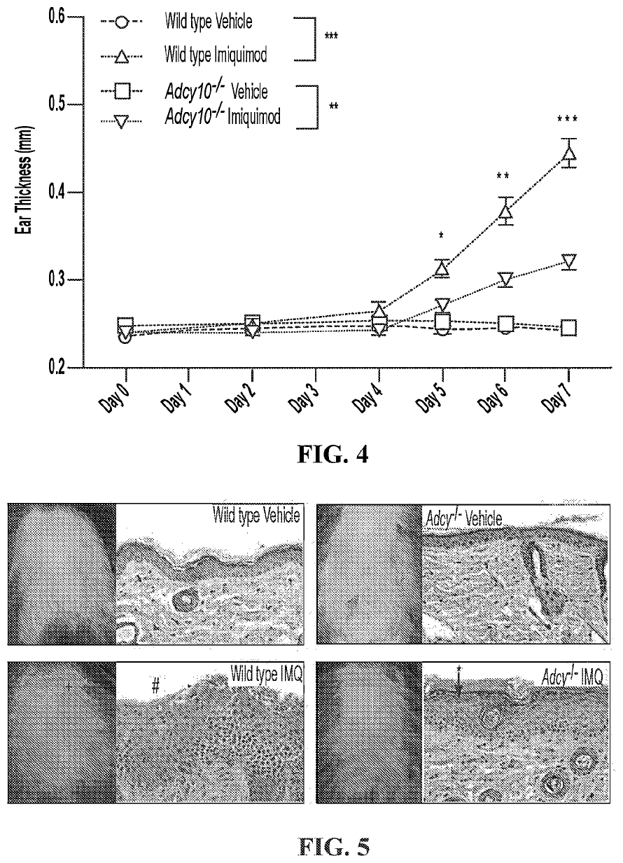

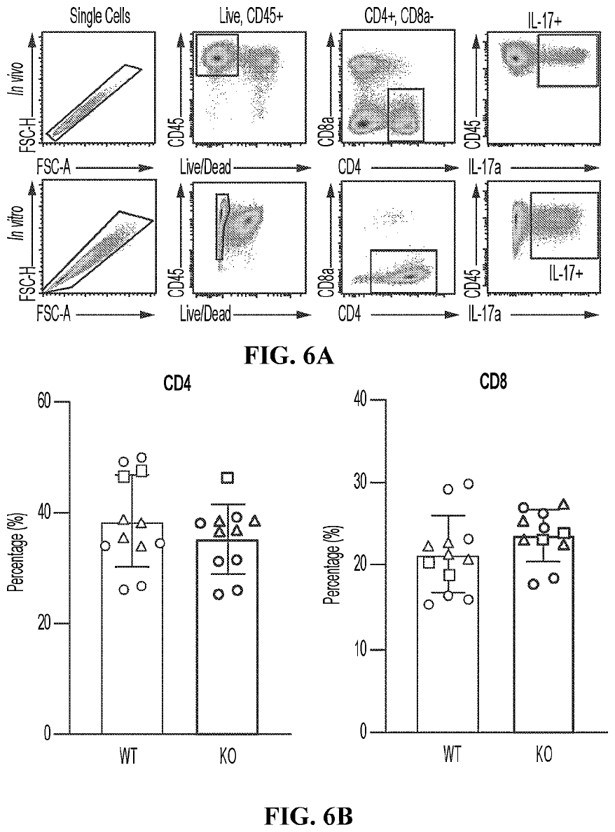

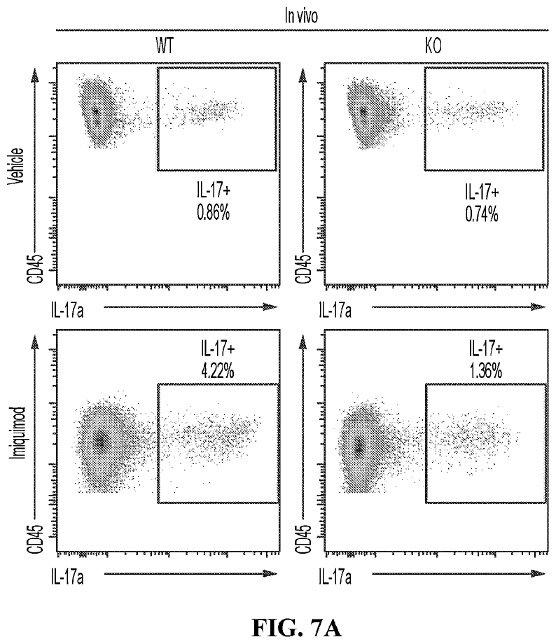

It has been previously reported that sAC was upregulated in human psoriatic lesions relative to normal skin and was expressed in multiple cell types including keratinocytes and T cells. These data suggested that sAC activity may be important for type 17 immune responses in skin. Imiquimod is a toll-like receptor 7 agonist and, when applied to mouse skin, induces a type 17 immune response, psoriasis-like dermatitis, and a gene expression profile very similar to human psoriasis. Application of imiquimod to back and ear skin led to significant inflammation in wild-type C57BL/6 mice as measured by increased erythema and scale formation on the back and swelling of the ear (FIG. 4, FIG. 5). In contrast, application of imiquimod to the skin of Adcy10^{-/-} mice led to significantly less erythema and scale formation on the back and reduced swelling of the ears (FIG. 4, FIG. 5). Histologic evaluation of the skin revealed that wild-type animals developed classic epidermal features of psoriasis including acanthosis, spongiosis, loss of the granular cell layer, and parakeratosis following application of imiquimod (FIG. 5, Left upper versus lower panel). In contrast, Adcy10^{t} skin developed less acanthosis, did not lose the granular cell layer, and did not exhibit parakeratosis, suggesting that the type 17 immune response in sAC mice is blunted (FIG. 5, Right upper panel vs lower panel). The type 17 immune response is defined by the expression of IL-17 due primarily to the generation of Th17 cells. Consistent with this observation, induction of IL17+ T cells was reduced in knockout (KO) relative to wild type (WT) mice in response to imiquimod (FIGS. 7A-7B and FIG. 6A). Of note, the diminished stimulation of IL17+ T cells in Adcy10 mice was not due to an overall reduction in T cell number and baseline CD4/CD8 cell number was similar between wild type and Adcy10 mice (FIG. 6B). The blunted induction of IL17+ T cells in Adcy10 mice following imiquimod treatment would be predicted to lead to a reduction in Th17-dependent gene expression in the skin.

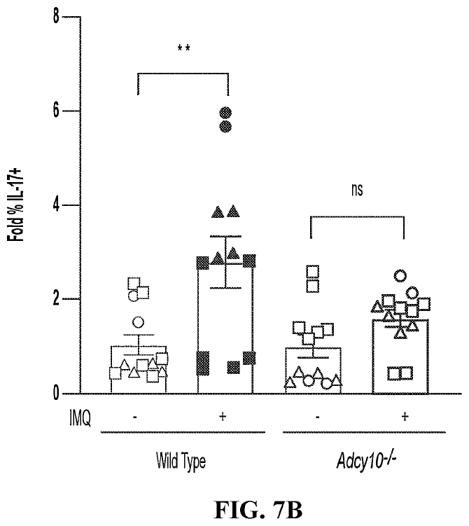

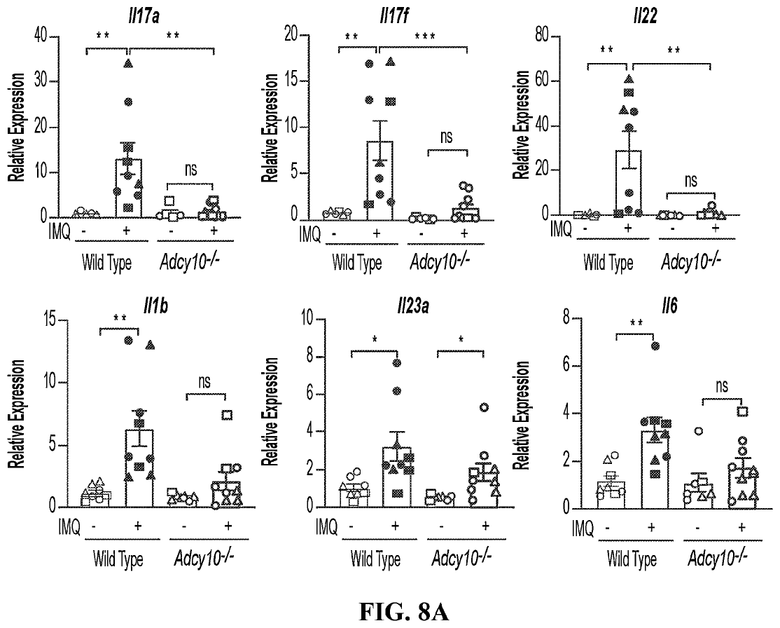

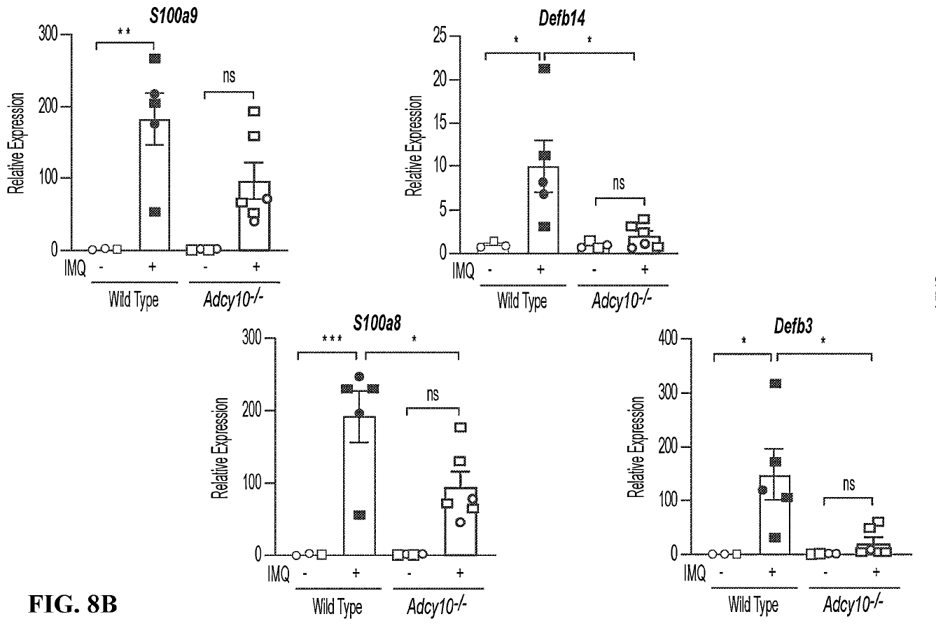

Imiquimod induces a significant increase in type 17 inflammatory gene expression in the skin of some strains of wild-type mice (FIGS. 8A-8B). Il17a, Il17f, and Il22 are expressed in Th17 and certain innate lymphoid cells and their expression defines type 17 inflammation. In contrast to wild-type mice, imiquimod did not induce the expression of Il17a, Il17f, or Il22 in the skin of Adcy10 mice (FIG. 8A). IL-1β, IL-23 and IL-6 are key cytokines responsible for the polarization and maintenance of Th17 cells. The genes that encode these cytokines, Il1b, Il23a, and Il6 were induced by imiquimod in wild-type murine skin (FIG. 8A); however, the ability of imiquimod to induce these genes in Adcy10^{-/-} skin was significantly blunted (FIG. 8A). IL-17 and IL-22 lead to stimulation of keratinocytes in the epidermis resulting in increased growth and reduced differentiation (FIG. 5). The keratinocyte genes S100a8, S100a9, Defb3 and Defb14 are upregulated by IL-17 and IL-22, and are keratinocyte markers of type 17 inflammation. Imiquimod potently induced these keratinocyte genes in wild-type but not in Adcy10 skin (FIG. 8B). Thus, type 17 inflammatory genes induced by imiquimod are suppressed in Adcy10 mice. Imiquimod-induced inflammation in the skin is a multicellular process. cAMP signaling can affect both T cells and keratinocytes; therefore, it was asked if sAC was required for the activation of these cells.

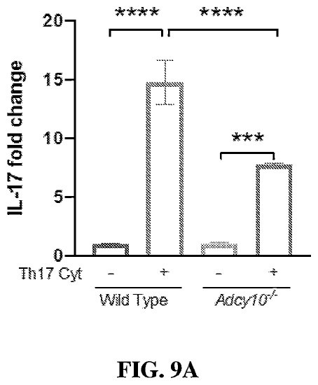

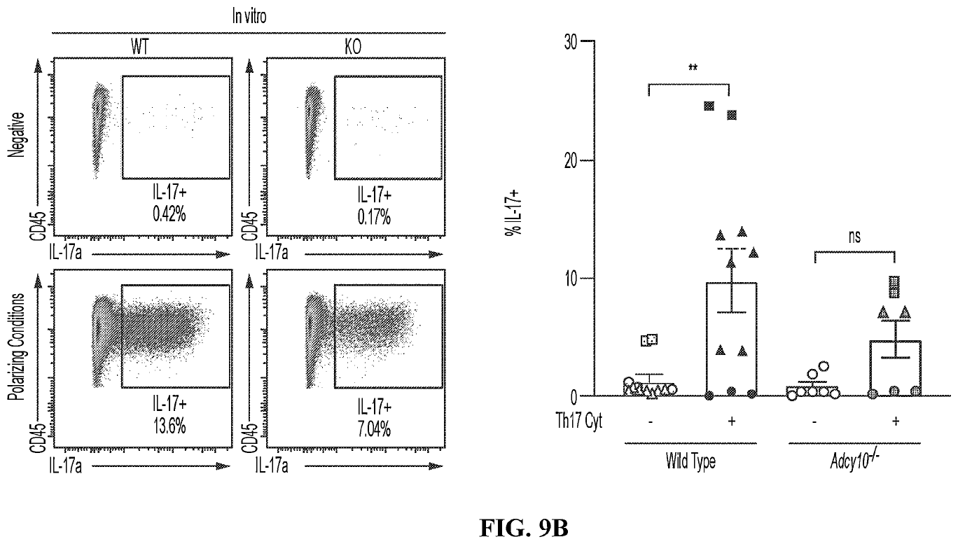

T cells can be polarized into different Th cell subsets in vitro by stimulating their T cell receptor (TCR) while exposing them to specific cytokines. The cytokines IL-1β, IL-6 and IL-23 promote the generation of Th17 cells. T cells were isolated from the spleens of untreated wild type and Adcy10^{-/-} animals and were incubated with anti-CD3/anti-CD28 antibodies to activate the cells in the presence or absence of the cytokines IL-1β, IL-6 and IL2-3 for four days to induce Th17 cell polarization. Under these culture conditions, wild-type T cells derived from the spleen differentiated into Th17 cells as evidenced by a near 15-fold increase in IL-17 secretion and a significant increase in CD45+, CD4+, IL17+ T cells as measured by flow cytometry (FIGS. 9A-9B). In contrast, Adcy10 T cell secretion of IL-17 was significantly reduced following growth in Th17 cell polarizing conditions (FIG. 9A). Consistent with reduced IL-17 secretion by Adcy10^{-/-} T cells under Th17 polarizing conditions, flow cytometry revealed that the generation of IL-17+CD45+ T cells was significantly blunted in Adcy10 T cells (FIG. 9B). Thus, sAC activity is required for normal Th17 cell polarization. To confirm whether the blunted type 17 immune response observed in Adcy10 mice (FIG. 4, FIG. 5) might also be due to a defect in keratinocytes, isolated human keratinocytes were examined following stimulation with type 17 inflammatory cytokines. The cytokines IL-17 and IL-22 recruit inflammatory cells and induce the proliferation of keratinocytes, respectively, in skin during psoriasis. N/Tert human keratinocytes were treated with IL-17/IL-22 and measured the expression of psoriasis disease associated genes. IL-17/IL-22 treatment of N/Tert human keratinocytes induces the expression of genes upregulated in psoriatic skin lesions such as S100a7 and Lcn2. LRE1, a specific inhibitor of sAC, was used to test whether sAC activity is necessary for this effect. In contrast to Th17 cell polarization, sAC activity appears to be dispensable for keratinocyte activation under type 17 inflammatory conditions. Thus, the data suggests that the reduced type 17 immune skin responses in mice is not due to a changes in keratinocyte response and is likely mainly the result of sAC-dependent signaling in T cells.

Th17 cell polarization requires the expression of lineage specific transcription factors such as RORc. Numerous reports have shown that cAMP signaling can influence gene expression in Th17 cells but the specific source of cAMP has remained unknown. Next, it was determined whether sAC activity affected gene expression during Th17 polarization. T cells derived from the spleen of wild-type and Adcy10 mice were cultured in anti-CD3/anti-CD28 antibodies in the presence or absence of the cytokines IL-1β, IL-6 and IL-23. Th17 polarizing conditions led to significant gene expression in both wild-type and Adcy10^{t} T cells. Specifically, Th17 polarizing conditions led to significant and potent changes in the expression of over 100 genes in both wild type and Adcy10^{-/-} T cells (>two-fold change; padj <0.01). However, comparative analysis of wild type and Adcy10 T cell gene expression profiles revealed significant differences. Wild-type T cells had 33 genes induced by Th17 polarizing conditions which were unaffected in Adcy10 T cells; these include genes known to be critical for Th17 activation (Cxc12, Il1a, and Il1b). Broad analysis of immunology gene sets identified 447 gene signatures that were positively enriched with a FDR <0.25 (p<1%); whereas, the same analysis identified only 240 significant gene signatures in Adcy10^{-/-} T cells. Specific examination of Th17 gene expression GSEA revealed that only the wild-type T cell gene expression profile was significantly enriched for Th17-dependent gene expression. Interestingly, even though the Th17 gene expression profile was suppressed in Adcy10 T cells relative to wild-type T cells, the induction of lineage-defining transcription factors and regulators, such as RORc and cMAF, were unaffected. Cyclic adenosine monophosphate binding protein (CREB) is known to affect Th17-dependent genes downstream of RORc expression without affecting the expression of Th17 lineage-defining transcription factors. Using an established CREB-dependent gene expression profile, it was revealed that CREB-dependent gene expression following IL-1β, IL-6 and IL23 stimulation was inhibited in Adcy10^{-/-} as compared to wild-type T cells. Further analysis revealed numerous IL-1β, IL- and IL-23 stimulated, CREB-dependent genes that are significantly inhibited or not expressed in Adcy10^{-/-} T cells. Many of these genes (Cebpb, Crem, Il1a, Il1r1, and Slc7a2) are known to impact Th17 cell activation.

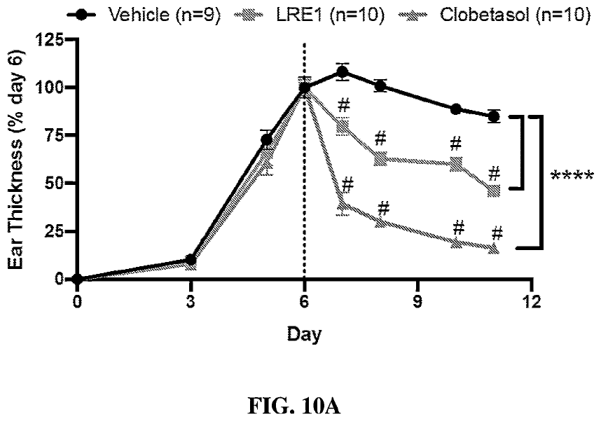

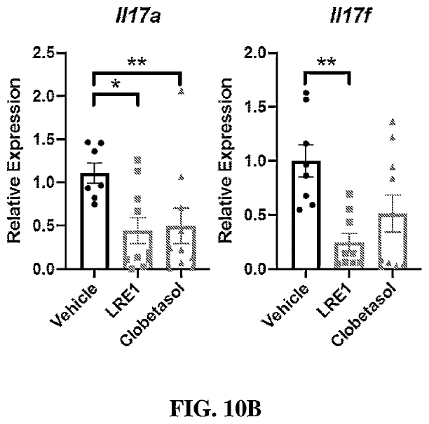

There are limited options for topical therapeutics for type 17 inflammatory diseases. Topical steroids are most commonly prescribed but can have significant adverse effects. Next, it was asked if sAC inhibitors could function as a topical treatments for type 17 inflammatory disease. The ideal therapeutic for type 17 skin inflammation would be one that could be applied to the skin when the disease was maximally active and would induce disease resolution. To induce type 17 inflammation in murine skin, wild-type mice were treated with imiquimod for six days to create psoriasis-like inflammation (FIGS. 10A-10B). After psoriasis-like inflammation was induced, mice were randomized into three cohorts to be treated with either vehicle, sAC inhibitor (LRE1), or clobetasol twice a day. Imiquimod was continuously applied daily to maintain type 17 inflammation during drug treatment thereby mimicking real disease.

Whereas vehicle had no effect, both LRE1 and Clobetasol led to a significant reduction in skin inflammation (FIGS. 10A-10B). In addition, both LRE1 and Clobetasol led to significant reductions in Il17a and Il17f expression in the skin confirming that sAC inhibitors, similar to topical steroids, reduce type 17 inflammation in the skin. Histologic examination of skin revealed no evidence of cell death or toxicity.

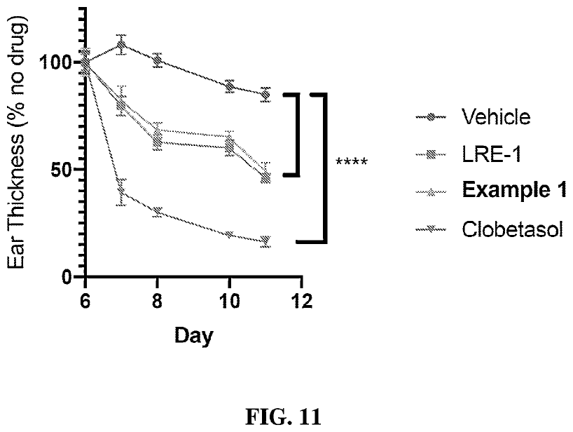

Additionally, as shown in FIG. 11, Example 1 also led to significant reduction in skin inflammation in mice with imiquimod-induced psoriasis-like inflammation.

Thus, topical application of sAC inhibitors is an effective method for reducing type 17 inflammation in mice. In general, sAC inhibitors, including those described herein, can be used to treat a variety of diseases and conditions associated with Th17-mediated immune response and/or type 17 inflammation.

### Discussion

sAC activity has been identified as essential for type 17 inflammation in vivo. The data suggest that sAC functions to support Th17 cell differentiation and does not have a significant role in the keratinocyte response to IL17 and IL22. sAC is not required for normal T cell development as total T cell and CD4/CD8 ratio is similar between WT and Adcy10 animals; however, sAC was required for the polarization of isolated T cells, which confirms a critical role of sAC in Th17 cell activation. The data confirms past reports that have identified cAMP signaling as an important signal during Th17 cell activation. In those reports the source of cAMP was not identified; thus, this work fills an important gap in the understanding of the mechanism of cAMP-dependent regulation of Th17 cell activation.

Th17 cells are known to be regulated by extracellular pH and metabolism. sAC is a sensor of bicarbonate ions and its activity reflects changes in pH and metabolism. Therefore, sAC may provide a link between these environmental changes and Th17 activity.

Additionally, Th17 cells have an important role in numerous diseases including, but not limited to, gastrointestinal diseases, rheumatic diseases, diseases of the central nervous system, acute respiratory distress syndrome, and systemic inflammatory diseases such as systemic lupus erythematosus. In each of these diseases, cAMP and/or CREB-dependent gene expression has been demonstrated to play a role. sAC-dependent cAMP likely has a role in many other Th17 mediated diseases.

At present, there is a relative dearth of effective topical medications for Th17 diseases. sAC inhibitors have been identified as a potential therapeutic approach. sAC inhibitors have been used in multiple mouse models with no obvious toxicity. The application of sAC inhibitors to the skin for weeks with no epidermal toxicity was demonstrated previously, and reported similar results. A head-to-head comparison was performed of sAC inhibitors with the class 1 corticosteroid clobetasol and it was found that sAC inhibitors led to a statistically significant reduction in Th17-dependent inflammation at near clobetasol potency.

### Experimental Methods

### In Vitro Th17 Cell Polarization

Spleens from male adcy10^{-/-} and wild type C57B1/6 mice age 8-10 weeks were excised and mechanically ground to obtain single cell suspensions. Cells were treated with a red blood cell lysis buffer (155 mM Ammonium chloride, 10 mM sodium bicarbonate, 0.1 mM EDTA in PBS pH 7.4) for two minutes and enriched for CD4+ T cells using a magnetic bead negative selection (Miltenyi). T cells were cultured in 96-well plates coated the night before with 200 uL of 10 μg/mL anti-CD3 antibody (BD biosciences) in IMDM modified with 1% sodium pyruvate, 1% L-glutamine, 1% pen/strep, and 10% FBS (Thermo Fischer), in the presence of 2 μg/mL anti-CD28 antibody (BD biosciences), 20 ng/mL recombinant mouse IL-1 beta (Miltenyi), 25 ng/mL IL-6 (Miltenyi), and 20 ng/mL IL-23 (R&D Systems). Media was also supplemented with 1 μg/mL anti-IFN gamma antibody (Thermo Fisher) and 1 μg/mL anti IL-4 antibody (Thermo Fisher) to inhibit Th1 and Th2 polarization, respectively. Cells were used as follows for different analyses. After 18 hours in culture, cells were collected for RNA sequencing. Eighteen hours was chosen for RNAseq because qPCR analysis found this time point to be the peak of RORc expression and deemed appropriate to examine early transcriptional changes during Th17 differentiation. In parallel after four days in culture, supernatant was collected and ELISA was performed to measure secreted cytokines. In parallel, after four days in culture cells were treated with PMA and Ionomycin and GolgiStop (BD) for 4 hours and flow cytometry analysis was performed.

### Imiquimod Stimulation of Mice

Male adcy10^{-/-} and wild type C57B1/6 mice age 8-11 weeks had their flanks shaved and remaining hair removed with Nair. The following day baseline ear caliper measurements and reference pictures were taken. Mice were treated daily with 62.5 mg of 5% imiquimod cream (Taro Pharmaceuticals) on the flank, and on both ears for 6 days. On days 3 and 4, 100 μL of saline solution was injected intraperitoneally into each mouse to prevent dehydration. Ear thickness measurements were recorded using a mitutyo digital caliper on days 2, 4, 5, 6, and on 7. Pictures were taken on days 3, 6, and 7. On day 7 mice were euthanized. Ears were removed and a 5 mm skin punch excision was performed from the treated area of the flank. Tissue samples were stored in RNAlater (Sigma) for molecular analysis, or fixed in 10% formalin for histology. CD4+ T cells were isolated as above, treated for four hours with PMA, ionomycin and golgi stop and analyzed by flow cytometry.

### Evaluation of sAC Inhibitors Following Imiquimod Treatment

Wild type C57B1/6 male mice 8-10 week old were purchased from Jackson labs. A 7 mm diameter circle was shaved and treated with Nair on the flank. Baseline ear thickness was measured. For 5 days, mice received daily treatment of 30 mg of 5% imiquimod cream (Taro) to each ear and to the 7 mm area on the flank. Ear thickness was recorded on days 3 and 5 to ensure peak inflammation had occurred. Mice were then randomized into three cohorts: vehicle, LRE1, and Clobetasol. For the next 6 days, mice were treated twice a day with 30 μL of either vehicle at a 1:1 PEG 400 to DMSO mixture, 3% LRE-1 in a 1:1 PEG 400 to DMSO mixture, 1.5% Example 1 in a 1:1 PEG400, or 0.05% clobetasol propionate (Sigma) in 1:1 PEG 400 to DMSO mixture on both ears and the flank. Treatment with imiquimod was continued during this period as above. Ear thickness was recorded every day before the first dose of drug. On day 11, all mice were euthanized. A 7 mm punch biopsy was collected from the flank and both ears were removed. Skin was stored in RNAlater (Sigma) for qPCR or 10% formalin for immunohistochemistry.

### Quantitative RT-PCR

For tissue, RNA was isolated according to specifications of the RNA easy plus universal mini kit (Qiagen). Tissue was homogenized using stainless steel beads in a bead beater. For frozen cell pellets, RNA was isolated according to specifications of the RNA easy plus mini kit (Qiagen). Samples were homogenized using Qiashredder columns (Qiagen). RNA quality control was performed using a nanodrop spectrophotometer. cDNA was made using the high-capacity rna-to-cdna kit (thermo fischer). The applied biosystems power SYBR green PCR master mix was used for qPCR. 40 cycles with annealing temperatures of 60 degrees celsius and a melting curve were performed using the QuantStudio 6 real-time PCR instrument (thermo fischer). Delta delta CT analysis was performed to determine relative transcription amongst samples normalized to GAPDH.

### ELISA for Cytokine Measurement

IL-17, IL-6, IL-9, and IL-4 concentrations were determined using DuoSet ELISA kits (R&D Systems). IL-22 was determined using an Antigenix America kit, and IFN gamma was determined using a thermo fischer kit. TMB substrate reagent (BD Biosciences) and 2N sulfuric acid (VWR) was used to stop the reaction. If samples were too concentrated for the standard curve of the assay, samples were diluted.

### Flow Cytometry

Flow cytometric analysis was performed on a Becton-Dickinson Fortessa analyzer. Lymphocytes were isolated from lymph nodes. All cells were stained with LIVE/DEAD® Fixable Dead Cell Stain (Life Technologies). Direct ex vivo staining was conducted with fluorochrome-labelled antibodies. For intracellular staining, cells were surface-stained, permeabilized and fixed before staining intracellular targets using the Foxp3 Permeabilization/Fixation Kit (eBioscience) according to the manufacturer's instructions. All antibodies and clones used are summarized in the table below. Flow cytometry data were analyzed with FlowJo software (TreeStar).

### Antibodies and Reagents Used in Flow Cytometry

| Target | Clone | Provider |
|---|---|---|
| IFN-g | XMG1.2 | ThermoFisher |
| TCRgd | GL3 | Biolegend |
| IL-5 | TRFK5 | BD Biosciences |
| IL-17A | TC11-18H10.1 | Biolegend |
| IL-9 | RM9A4 | Biolegend |
| CD45 | 30-F11 | ThermoFisher |
| TCRb | H57-597 | Biolegend |
| Foxp3 | FJK-16s | ThermoFisher |
| Live/Dead | L34965 | ThermoFisher |
| CD4 | RM4-5 | Biolegend |
| CD8a | 53-6.7 | BD Biosciences |
| IL-22 | IL22JOP | eBioscience |

### REFERENCES

1. Division of Reproductive Health, National Center for Disease Prevention and Health Promotion. Birth Control Methods. In: Centers for Disease Control and Prevention; 2018.
2. Amory J K. Male contraception. Fertil Steril 2016; 106:1303-1309.
3. O'Rand M G, Silva E J, Hamil K G. Non-hormonal male contraception: A review and development of an Eppin based contraceptive. Pharmacol Ther 2016; 157:105-111.
4. Kamenetsky M, Middelhaufe S, Bank E M, Levin L R, Buck J, Steegborn C. Molecular details of cAMP generation in mammalian cells: a tale of two systems. J Mol Biol 2006; 362:623-639.
5. Buck J, Sinclair M L, Schapal L, Cann M J, Levin L R. Cytosolic adenylyl cyclase defines a unique signaling molecule in mammals. Proc Natl Acad Sci USA 1999; 96:79-84.
6. Neer E J. Multiple forms of adenylate cyclase. Adv Cyclic Nucleotide Res 1978; 9:69-83.
7. Braun T, Dods R F. Development of a Mn-2+-sensitive, “soluble” adenylate cyclase in rat testis. Proc Natl Acad Sci USA 1975; 72:1097-1101.
8. Braun T, Frank H, Dods R, Sepsenwol S. Mn2+-sensitive, soluble adenylate cyclase in rat testis. Differentiation from other testicular nucleotide cyclases. Biochim Biophys Acta 1977; 481:227-235.
9. Gordeladze J O, Abyholm T, Cusan L, Clausen O P, Hansson V. Cellular localization of the Mn2+-dependent adenylyl cyclase in the human testis. Arch Androl 1982; 8:199-204.
10. Gordeladze J O, Purvis K, Clausen O P, Rommerts F F, Hansson V. Cellular localization of the Mn2+-dependent adenylyl cyclase (AC) in rat testis. Int J Androl 1981; 4:172-182.
11. Garbers D L, Kopf G S. The regulation of spermatozoa by calcium and cyclic nucleotides. Adv Cyclic Nucleotide Res 1980; 13:251-306.
12. Garty N B, Salomon Y. Stimulation of partially purified adenylate cyclase from bull sperm by bicarbonate. FEBS Lett 1987; 218:148-152.
13. Visconti P E, Kopf G S. Regulation of protein phosphorylation during sperm capacitation. Biol Reprod 1998; 59:1-6.
14. Visconti P E, Stewart-Savage J, Blasco A, Battaglia L, Miranda P, Kopf G S, Tezon J G. Roles of bicarbonate, cAMP, and protein tyrosine phosphorylation on capacitation and the spontaneous acrosome reaction of hamster sperm. Biol Reprod 1999; 61:76-84.
15. Chaloupka J A, Bullock S A, Iourgenko V, Levin L R, Buck J. Autoinhibitory regulation of soluble adenylyl cyclase. Mol Reprod Dev 2006; 73:361-368.
16. Middelhaufe S, Leipelt M, Levin L R, Buck J, Steegborn C. Identification of a haem domain in human soluble adenylate cyclase. Biosci Rep 2012; 32:491-499.
17. Danot O, Marquenet E, Vidal-Ingigliardi D, Richet E. Wheel of Life, Wheel of Death: A Mechanistic Insight into Signaling by STAND Proteins. Structure 2009; 17:172-182.
18. Jaiswal B S, Conti M. Identification and functional analysis of splice variants of the germ cell soluble adenylyl cyclase. J Biol Chem 2001; 276:31698-31708.
19. Jaiswal B S, Conti M. Calcium regulation of the soluble adenylyl cyclase expressed in mammalian spermatozoa. Proc Natl Acad Sci USA 2003; 100:10676-10681.
20. Chen Y, Cann M J, Litvin T N, Iourgenko V, Sinclair M L, Levin L R, Buck J. Soluble adenylyl cyclase as an evolutionarily conserved bicarbonate sensor. Science 2000; 289:625-628.
21. Litvin T N, Kamenetsky M, Zarifyan A, Buck J, Levin L R. Kinetic properties of “soluble” adenylyl cyclase. Synergism between calcium and bicarbonate. J Biol Chem 2003; 278:15922-15926.
22. Stengel D, Guenet L, Desmier M, Insel P, Hanoune J. Forskolin requires more than the catalytic unit to activate adenylate cyclase. Mol Cell Endocrinol 1982; 28:681-690.
23. Kleinboelting S, Diaz A, Moniot S, van den Heuvel J, Weyand M, Levin L R, Buck J, Steegborn C. Crystal structures of human soluble adenylyl cyclase reveal mechanisms of catalysis and of its activation through bicarbonate. Proc Natl Acad Sci USA 2014; 111:3727-3732.
24. Forte L R, Bylund D B, Zahler W L. Forskolin does not activate sperm adenylate cyclase. Mol Pharmacol 1983; 24:42-47.
25. Zippin J H, Chen Y, Straub S G, Hess K C, Diaz A, Lee D, Tso P, Holz G G, Sharp G W, Levin L R, Buck J. CO2/HCO3(−)- and calcium-regulated soluble adenylyl cyclase as a physiological ATP sensor. J Biol Chem 2013; 288:33283-33291.
26. Balbach M, Beckert V, Hansen J N, Wachten D. Shedding light on the role of cAMP in mammalian sperm physiology. Mol Cell Endocrinol 2018; 468:111-120.
27. Buffone M G, Wertheimer E V, Visconti P E, Krapf D. Central role of soluble adenylyl cyclase and cAMP in sperm physiology. Biochim Biophys Acta 2014; 1842:2610-2620.
28. Austin C R. Activation and the correlation between male and female elements in fertilization. Nature 1951; 168:558-559.
29. Chang M C. Fertilizing capacity of spermatozoa deposited into the fallopian tubes. Nature 1951; 168:697-698.
30. Levine N, Kelly H. Measurement of pH in the rat epididymis in vivo. J Reprod Fertil 1978; 52:333-335.
31. Wennemuth G, Carlson A E, Harper A J, Babcock D F. Bicarbonate actions on flagellar and Ca²⁺-channel responses: initial events in sperm activation. Development 2003; 130:1317-1326.
32. Carlson A E, Quill T A, Westenbroek R E, Schuh S M, Hille B, Babcock D F. Identical phenotypes of CatSper1 and CatSper2 null sperm. J Biol Chem 2005; 280:32238-32244.
33. Garbers D L, Tubb D J, Hyne R V. A requirement of bicarbonate for Ca²⁺-induced elevations of cyclic AMP in guinea pig spermatozoa. J Biol Chem 1982; 257:8980-8984.
34. Rojas F J, Bruzzone M E, Moretti-Rojas I. Regulation of cyclic adenosine monophosphate synthesis in human ejaculated spermatozoa. II. The role of calcium and bicarbonate ions on the activation of adenylyl cyclase. Hum Reprod 1992; 7:1131-1135.
35. Esposito G, Jaiswal B S, Xie F, Krajnc-Franken M A, Robben T J, Strik A M, Kuil C, Philipsen R L, van Duin M, Conti M, Gossen J A. Mice deficient for soluble adenylyl cyclase are infertile because of a severe sperm-motility defect. Proc Natl Acad Sci USA 2004; 101:2993-2998.
36. Chen J, Martinez J, Milner T A, Buck J, Levin L R. Neuronal expression of soluble adenylyl cyclase in the mammalian brain. Brain Res 2013; 1518:1-8.
37. Hess K C, Jones B H, Marquez B, Chen Y, Ord T S, Kamenetsky M, Miyamoto C, Zippin J H, Kopf G S, Suarez S S, Levin L R, Williams C J, et al. The “soluble” adenylyl cyclase in sperm mediates multiple signaling events required for fertilization. Dev Cell 2005; 9:249-259.
38. Xie F, Garcia M A, Carlson A E, Schuh S M, Babcock D F, Jaiswal B S, Gossen J A, Esposito G, van Duin M, Conti M. Soluble adenylyl cyclase (sAC) is indispensable for sperm function and fertilization. Dev Biol 2006; 296:353-362.
39. Akbari A, Pipitone G B, Anvar Z, Jaafarinia M, Ferrari M, Carrera P, Totonchi M. ADCY10 frameshift variant leading to severe recessive asthenozoospermia and segregating with absorptive hypercalciuria. Hum Reprod 2019; 34:1155-1164.
40. Steegborn C, Litvin T N, Hess K C, Capper A B, Taussig R, Buck J, Levin L R, Wu H. A novel mechanism for adenylyl cyclase inhibition from the crystal structure of its complex with catechol estrogen. J Biol Chem 2005; 280:31754-31759.
41. Bitterman J L, Ramos-Espiritu L, Diaz A, Levin L R, Buck J. Pharmacological distinction between soluble and transmembrane adenylyl cyclases. J Pharmacol Exp Ther 2013; 347:589-598.
42. Pastor-Soler N, Beaulieu V, Litvin T N, Da Silva N, Chen Y, Brown D, Buck J, Levin L R, Breton S. Bicarbonate-regulated adenylyl cyclase (sAC) is a sensor that regulates pH-dependent V-ATPase recycling. J Biol Chem 2003; 278:49523-49529.
43. Lee Y S, Tresguerres M, Hess K, Marmorstein L Y, Levin L R, Buck J, Marmorstein A D.

Regulation of anterior chamber drainage by bicarbonate-sensitive soluble adenylyl cyclase in the ciliary body. J Biol Chem 2011; 286:41353-41358.
44. Wiggins S V, Steegborn C, Levin L R, Buck J. Pharmacological modulation of the CO2/HCO3(−)/pH-, calcium-, and ATP-sensing soluble adenylyl cyclase. Pharmacol Ther 2018; 190:173-186.
45. Tian G, Sandler S, Gylfe E, Tengholm A. Glucose- and Hormone-Induced cAMP Oscillations in α- and β-Cells Within Intact Pancreatic Islets. Diabetes 2011; 60:1535-1543.
46. Ramos-Espiritu L, Kleinboelting S, Navarrete F A, Alvau A, Visconti P E, Valsecchi F, Starkov A, Manfredi G, Buck H, Adura C, Zippin J H, van den Heuvel J, et al. Discovery of LRE1 as a specific and allosteric inhibitor of soluble adenylyl cyclase. Nat Chem Biol 2016; 12:838-844.
47. Sinclair M L, Wang X Y, Mattia M, Conti M, Buck J, Wolgemuth D J, Levin L R. Specific expression of soluble adenylyl cyclase in male germ cells. Mol Reprod Dev 2000; 56:6-11.
48. Farrell J, Ramos L, Tresguerres M, Kamenetsky M, Levin L R, Buck J. Somatic ‘soluble’ adenylyl cyclase isoforms are unaffected in Sacy tmlLex/Sacy tmlLex ‘knockout’ mice. PLoS One 2008; 3:e3251.
49. Geng W, Wang Z, Zhang J, Reed B Y, Pak C Y, Moe O W. Cloning and characterization of the human soluble adenylyl cyclase. Am J Physiol Cell Physiol 2005; 288:C1305-1316.
50. Levin L R, Buck J. Physiological roles of acid-base sensors. Annu Rev Physiol 2015; 77:347-362.
51. Tresguerres M, Levin L R, Buck J. Intracellular cAMP signaling by soluble adenylyl cyclase. Kidney Int 2011; 79:1277-1288.
52. Chang J C, Oude-Elferink R P. Role of the bicarbonate-responsive soluble adenylyl cyclase in pH sensing and metabolic regulation. Front Physiol 2014; 5:42.
53. Chen X, Baumlin N, Buck J, Levin L R, Fregien N, Salathe M. A soluble adenylyl cyclase form targets to axonemes and rescues beat regulation in soluble adenylyl cyclase knockout mice. Am J Respir Cell Mol Biol 2014; 51:750-760.
54. Schmid A, Sutto Z, Nlend M C, Horvath G, Schmid N, Buck J, Levin L R, Conner G E, Fregien N, Salathe M. Soluble adenylyl cyclase is localized to cilia and contributes to ciliary beat frequency regulation via production of cAMP. J Gen Physiol 2007; 130:99-109.
55. Gandhi J K, Roy Chowdhury U, Manzar Z, Buck J, Levin L R, Fautsch M P, Marmorstein A D. Differential Intraocular Pressure Measurements by Tonometry and Direct Cannulation After Treatment with Soluble Adenylyl Cyclase Inhibitors. J Ocul Pharmacol Ther 2017; 33:574-581.
56. Watson R L, Buck J, Levin L R, Winger R C, Wang J, Arase H, Muller W A. Endothelial CD99 signals through soluble adenylyl cyclase and PKA to regulate leukocyte transendothelial migration. J Exp Med 2015; 212:1021-1041.
57. Bender A T, Beavo J A. Cyclic nucleotide phosphodiesterases: molecular regulation to clinical use. Pharmacological reviews 2006; 58:488-520.
58. Steegborn C, Litvin T N, Levin L R, Buck J, Wu H. Bicarbonate activation of adenylyl cyclase via promotion of catalytic active site closure and metal recruitment. Nat Struct Mol Biol 2005; 12:32-37.
59. Saalau-Bethell S M, Berdini V, Cleasby A, Congreve M, Coyle J E, Lock V, Murray C W, O'Brien M A, Rich S J, Sambrook T, Vinkovic M, Yon J R, et al. Crystal structure of human soluble adenylate cyclase reveals a distinct, highly flexible allosteric bicarbonate binding pocket. ChemMedChem 2014; 9:823-832.
60. Kleinboelting S, Ramos-Espiritu L, Buck H, Colis L, van den Heuvel J, Glickman J F, Levin L R, Buck J, Steegborn C. Bithionol Potently Inhibits Human Soluble Adenylyl Cyclase through Binding to the Allosteric Activator Site. J Biol Chem 2016; 291:9776-9784.
61. Xie, F. et al. Soluble adenylyl cyclase (sAC) is indispensable for sperm function and fertilization. Dev Biol 296, 353-362 (2006).
62. Balbach, M. et al. Soluble adenylyl cyclase inhibition prevents human sperm functions essential for fertilization. Mol Hum Reprod 27, doi:10.1093/molehr/gaab054 (2021).
63. Fushimi, M. et al. Discovery of TDI-10229: A Potent and Orally Bioavailable Inhibitor of Soluble Adenylyl Cyclase (sAC, ADCY10). ACS Med Chem Lett 12, 1283-1287, doi:10.1021/acsmedchemlett.1c00273 (2021).
64. Levine, N. & Marsh, D. J. Micropuncture studies of the electrochemical aspects of fluid and electrolyte transport in individual seminiferous tubules, the epididymis and the vas deferens in rats. J Physiol 213, 557-570, doi:10.1113/jphysiol.1971.sp009400 (1971).
65. Pitts, R. Physiology of the Kidney and Body Fluids, Vol. 3 (1974).
66. Organisation, W. H. Vol. Sixth edition (2021).
67. R. J., B. 285-304 (Univ. Chigaco Press, The biology of the cervix, 1973).
68. Suarez, S. S. & Pacey, A. A. Sperm transport in the female reproductive tract. Hum Reprod Update 12, 23-37, doi:10.1093/humupd/dmi047 (2006).
69. Hanson, F. W. & Overstreet, J. W. The interaction of human spermatozoa with cervical mucus in vivo. Am J Obstet Gynecol 140, 173-178, doi:10.1016/0002-9378(81)90105-8 (1981).
70. Katz, D. F., Mills, R. N. & Pritchett, T. R. The movement of human spermatozoa in cervical mucus. J Reprod Fertil 53, 259-265, doi:10.1530/jrf.0.0530259 (1978).
71. Fox, C. A., Meldrum, S. J. & Watson, B. W. Continuous measurement by radio-telemetry of vaginal pH during human coitus. J Reprod Fertil 33, 69-75, doi:10.1530/jrf.0.0330069 (1973).
72. Abbe, C. R., Page, S. T. & Thirumalai, A. Male Contraception. Yale J Biol Med 93, 603-613 (2020).
73. Gava, G. & Meriggiola, M. C. Update on male hormonal contraception. Ther Adv Endocrinol Metab 10, 2042018819834846, doi:10.1177/2042018819834846 (2019).
74. Amory, J. K. Development of Novel Male Contraceptives. Clin Transl Sci 13, 228-237, doi:10.1111/cts.12708 (2020).
75. Thirumalai, A. & Page, S. T. Male Hormonal Contraception. Annu Rev Med 71, 17-31, doi:10.1146/annurev-med-042418-010947 (2020).
76. Chang, Z. et al. Triptonide is a reversible non-hormonal male contraceptive agent in mice and non-human primates. Nat Commun 12, 1253, doi:10.1038/s41467-021-21517-5 (2021).
77. O'Rand, M. G., Hamil, K. G., Adevai, T. & Zelinski, M. Inhibition of sperm motility in male macaques with EP055, a potential non-hormonal male contraceptive. PLoS One 13, e0195953, doi:10.1371/journal.pone.0195953 (2018).
78. O'Rand, M. G., Hamil, K. G., Adevai, T. & Zelinski, M. Inhibition of sperm motility in male macaques with EP055, a potential non-hormonal male contraceptive. PLoS One 13, e0195953, doi:10.1371/journal.pone.0195953 (2018).
79. FDA Insert, V. Vol. Reference ID: 3466301 (Food and Drug Administration, 1998).
80. Visconti, P. E. et al. Capacitation of mouse spermatozoa. I. Correlation between the capacitation state and protein tyrosine phosphorylation. Development 121, 1129-1137 (1995).
81. Levin, L. R. et al. The Drosophila learning and memory gene rutabaga encodes a Ca2+/Calmodulin-responsive adenylyl cyclase. Cell 68, 479-489 (1992).
82. Levin, L. R. & Reed, R. R. Identification of functional domains of adenylyl cyclase using in vivo chimeras. J Biol Chem 270, 7573-7579, doi:10.1074/jbc.270.13.7573 (1995).
83. Laemmli, U. K. Cleavage of structural proteins during the assembly of the head of bacteriophage T4. Nature 227, 680-685, doi:10.1038/227680a0 (1970).
84. Hansen, J. N., Rassmann, S., Jikeli, J. F. & Wachten, D. ⁻A Simple Analysis Software to Comprehensively Study Flagellar Beating and Sperm Steering. Cells 8, doi:10.3390/cells8010010 (2018).

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### Additional Embodiments

Additional embodiments provided herein are indicated by the following numbered paragraphs:

1. A compound of Formula (I):

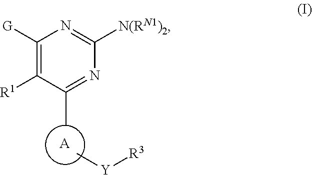

or a pharmaceutically acceptable salt thereof, wherein:
G is halogen, —CN, optionally substituted alkyl, or optionally substituted acyl;
R¹ is hydrogen, halogen, optionally substituted alkyl, or optionally substituted acyl;
A is an optionally substituted monocyclic heteroaryl ring comprising at least 1 nitrogen atom;
Y is a bond, optionally substituted alkylene, optionally substituted heteroalkylene, —O—, —NR^{N}, —S—, —S(═O)—, or —SO₂—;
R³ is optionally substituted carbocyclyl, optionally substituted heterocyclyl, optionally substituted aryl, or optionally substituted heteroaryl;
each instance of R^{N1} is independently hydrogen, optionally substituted alkyl, optionally substituted acyl, or a nitrogen protecting group, or optionally two R^{N1} are taken together with the intervening atoms to form optionally substituted heterocyclyl or optionally substituted heteroaryl;
provided that when G is not halogen, -(A)-Y—R³ is of the formula:

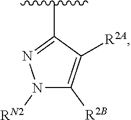

wherein:
R^{2A} and R^{2B} are independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, —SR^{S}, or —Y—R³
provided that one of R^{2A} and R^{2B} is —Y—R³;
R^{N2} is hydrogen, optionally substituted alkyl, optionally substituted acyl, or a nitrogen protecting group;
each instance of R^{N} is independently hydrogen, optionally substituted alkyl, optionally substituted acyl, or a nitrogen protecting group, or optionally two R^{N} are taken together with the intervening atoms to form optionally substituted heterocyclyl or optionally substituted heteroaryl;
each instance of R^{O} is independently hydrogen, optionally substituted alkyl, optionally substituted acyl, or an oxygen protecting group; and
each instance of R^{S} is independently hydrogen, optionally substituted alkyl, optionally substituted acyl, or a sulfur protecting group.
   2. The compound of paragraph 1, wherein A is an optionally substituted 5-membered heteroaryl ring comprising 2 or 3 nitrogen atoms.
   3. The compound of paragraph 1 or 2, wherein A is an optionally substituted pyrazole ring.
   4. The compound of any one of paragraphs 1-3, wherein the compound is of Formula (II):

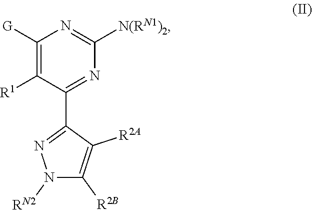

or a pharmaceutically acceptable salt thereof, wherein one of R^{2A} and R^{2B} is —Y—R³.
5. The compound of any one of paragraphs 1-4, wherein G is halogen.
6. The compound of any one of paragraphs 1-5, wherein G is —Cl.
7. The compound of any one of paragraphs 1-6, wherein R¹ is hydrogen.
8. The compound of any one of paragraphs 1-7, wherein the compound is of Formula (III):

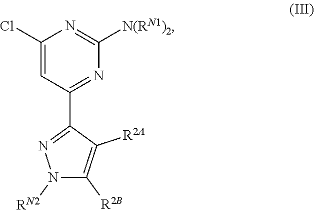

or a pharmaceutically acceptable salt thereof, wherein one of R^{2A} and R^{2B} is —Y—R³.
9. The compound of any one of paragraphs 1-8, wherein the compound is of Formula (IV):

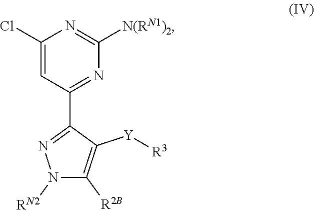

or a pharmaceutically acceptable salt thereof.
10. The compound of any one of paragraphs 1-9, wherein R³ is optionally substituted phenyl.
11. The compound of any one of paragraphs 1-10, wherein the compound is of Formula (V):

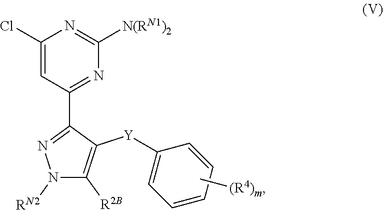

or a pharmaceutically acceptable salt thereof, wherein:
each instance of R⁴ is independently halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, or —SR^{S}; and
m is 0, 1, 2, 3, 4, or 5.
   12. The compound of any one of paragraphs 1-11, wherein Y is optionally substituted C₁₋₃ alkylene.
   13. The compound of any one of paragraphs 1-12, wherein Y is optionally substituted methylene.
   14. The compound of any one of paragraphs 1-12, the compound is of Formula (VI):

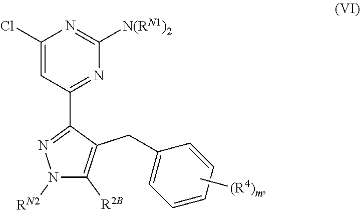

or a pharmaceutically acceptable salt thereof.
15. The compound of any one of paragraphs 1-14, wherein at least one instance of R^{N1} is hydrogen.
16. The compound of any one of paragraphs 1-15, wherein the compound is of the formula:

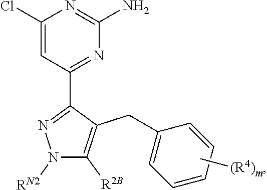

or a pharmaceutically acceptable salt thereof.
17. The compound of any one of paragraphs 11-16, wherein m is 1.
18. The compound of any one of paragraphs 1-17, wherein the compound is of the formula:

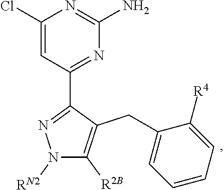

or a pharmaceutically acceptable salt thereof.
19. The compound of any one of paragraphs 11-18, wherein at least one instance of R⁴ is halogen.
20. The compound of paragraph 19, wherein at least one instance of R⁴ is —Cl or —F.
21. The compound of any one of paragraphs 11-20, wherein at least one instance of R⁴ is optionally substituted C₁₋₆ alkyl or optionally substituted C₁₋₆ acyl.
22. The compound of paragraph 21, wherein at least one instance of R⁴ is one of the following: —CO₂H, —CO₂Me, —CO₂CH₂Ph, —CH₂OCH₂CH₂NMe₂, —C(═O)NHCH₂Ph, —C(═O)NHMe, —C(═O)NHCH₂CH₂OMe, or —CO₂CH₂CH₂CH₂NMe₂; or is of the following formula:

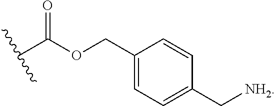 [SMILES: *C(=O)OCc1ccc(CN)cc1]

23. The compound of any one of paragraphs 11-22, wherein at least one instance of R⁴ is optionally substituted aryl or optionally substituted carbocyclyl.
24. The compound of paragraph 23, wherein at least one instance of R⁴ is of one of the following formulae:

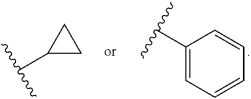

25. The compound of any one of paragraphs 11-24, wherein at least one instance of R⁴ is —OR^{O}.
26. The compound of paragraph 25, wherein at least one instance of R⁴ is one of the following: —OMe, —OCF₃, —OCH₂CO₂Me, —O(CH₂CH₂O)₃Me; or is of one of the following formulae:

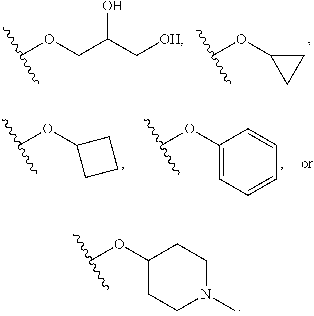

27. The compound of any one of paragraphs 11-26, wherein at least one instance of R⁴ is —Z—R⁵; wherein Z is a bond, optionally substituted alkylene, optionally substituted heteroalkylene, or optionally substituted acylene; and R⁵ optionally substituted heterocyclyl, optionally substituted heteroaryl, —N(R^{N})₂, or —OR^{O}.
28. The compound of paragraph 27, wherein Z is optionally substituted C₁₋₆ alkylene, optionally substituted C₁₋₆ heteroalkylene, or optionally substituted C₁₋₆ acylene.
29. The compound of paragraph 27 or 28, wherein Z is optionally substituted C₁₋₆ heteroalkylene.
30. The compound of paragraph 27 or 28, wherein Z is optionally substituted C₁₋₃ heteroalkylene.
31. The compound of paragraph 27 or 28, wherein Z is unsubstituted C₁₋₃ heteroalkylene.
32. The compound of paragraph 27 or 28, wherein Z is of one of the following formulae:

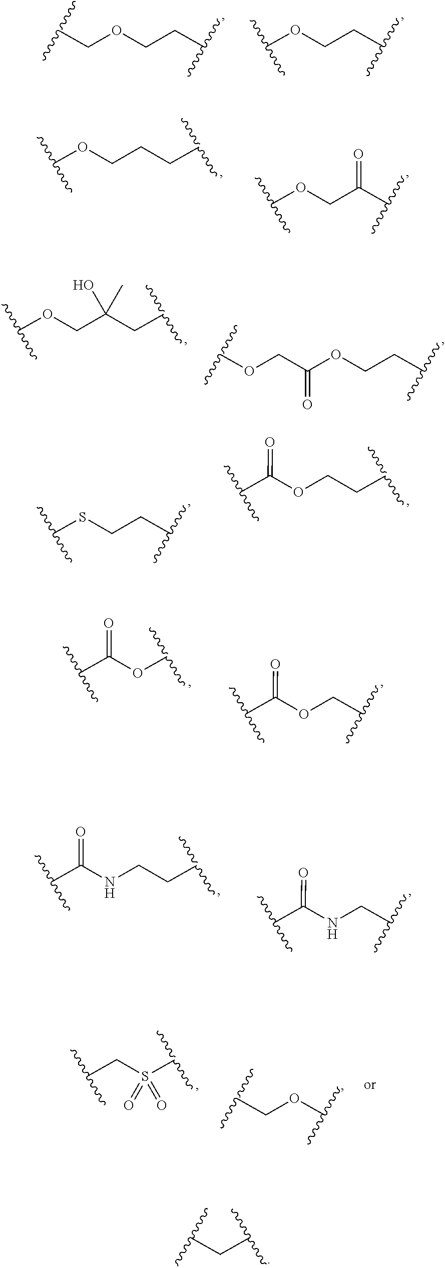

33. The compound of any one of paragraphs 27-32, wherein R⁵ is optionally substituted 4- to 7-membered heterocyclyl.
34. The compound of any one of paragraphs 27-34, wherein R⁵ is optionally substituted 5- or 6-membered heterocyclyl comprising 1 or 2 heteroatoms independently selected from N and O.
35. The compound of any one of paragraphs 27-32, wherein R⁵ is of one of the following formulae:

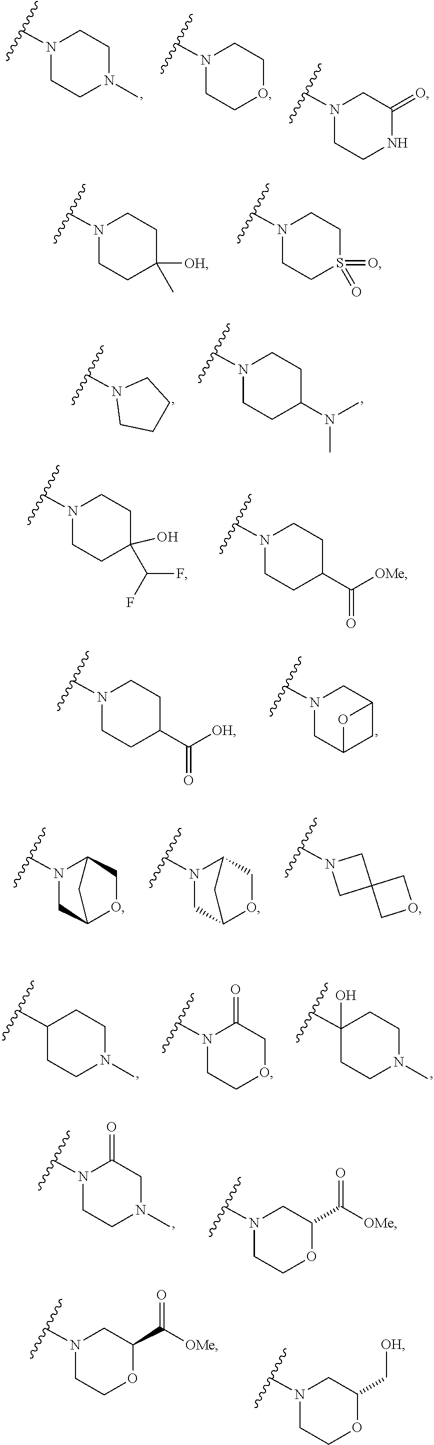

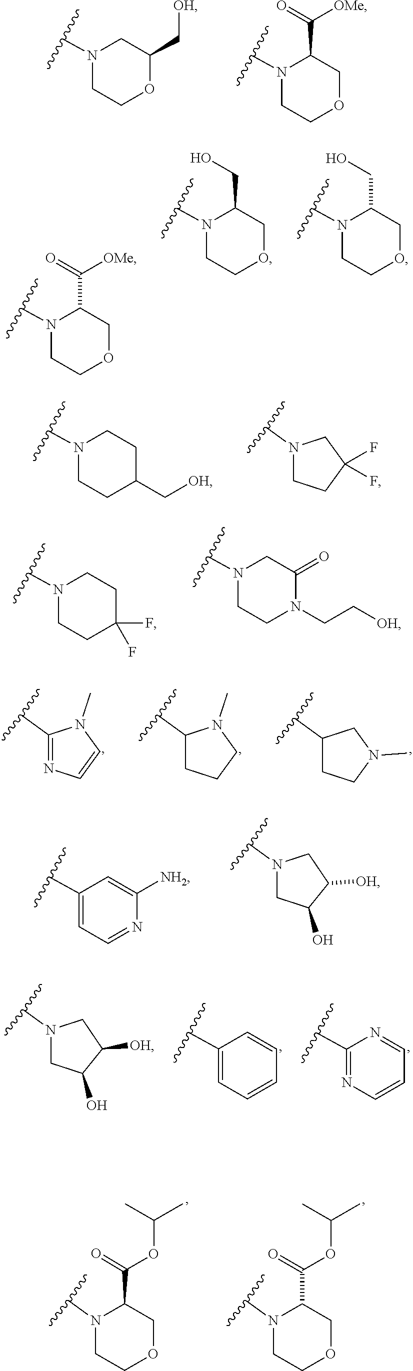

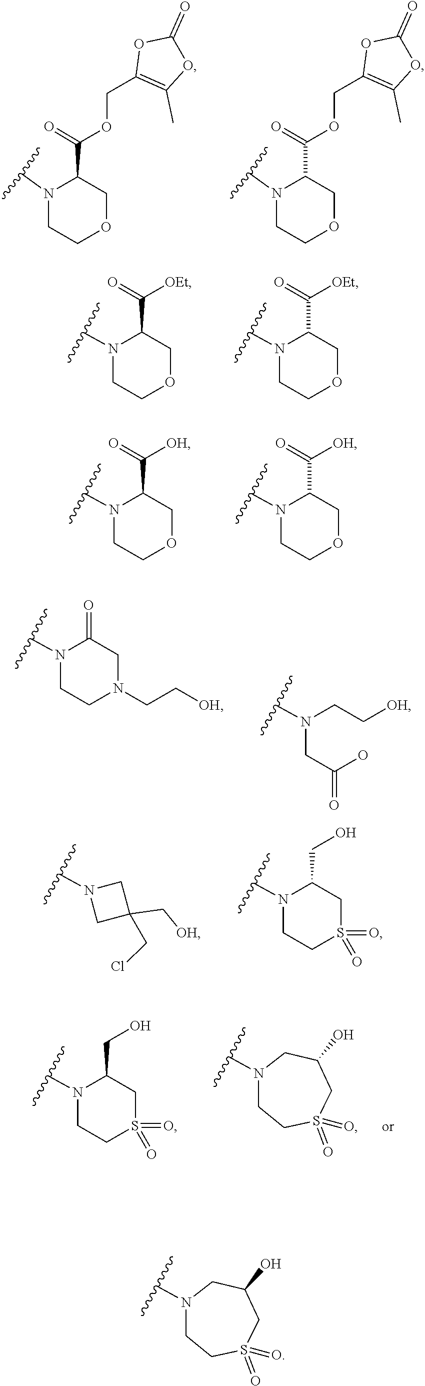

36. The compound of paragraph 27, wherein at least one instance of R⁴ is of one of the following formulae:

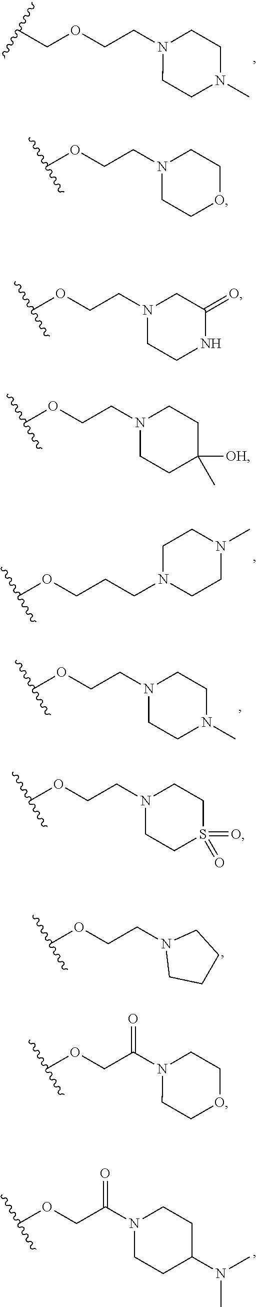

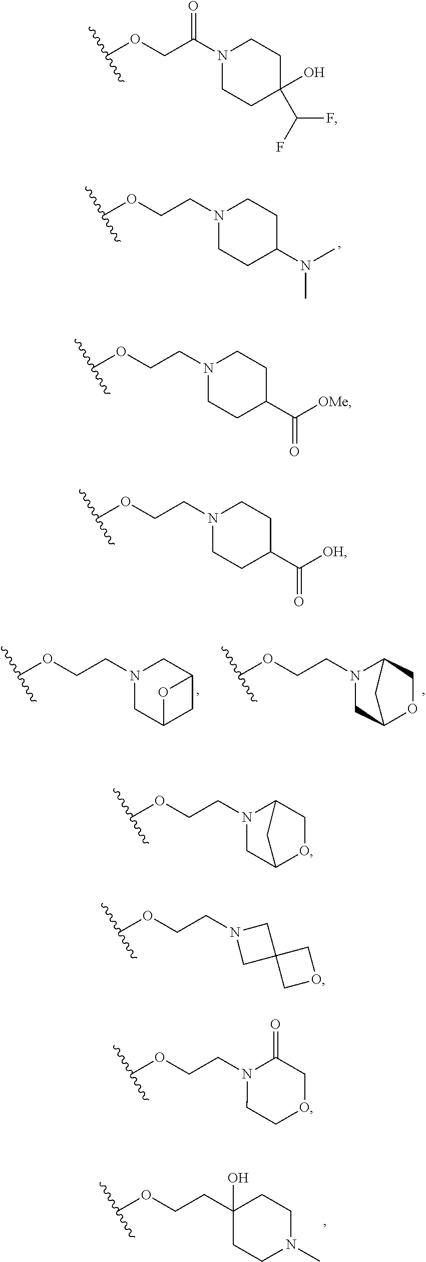

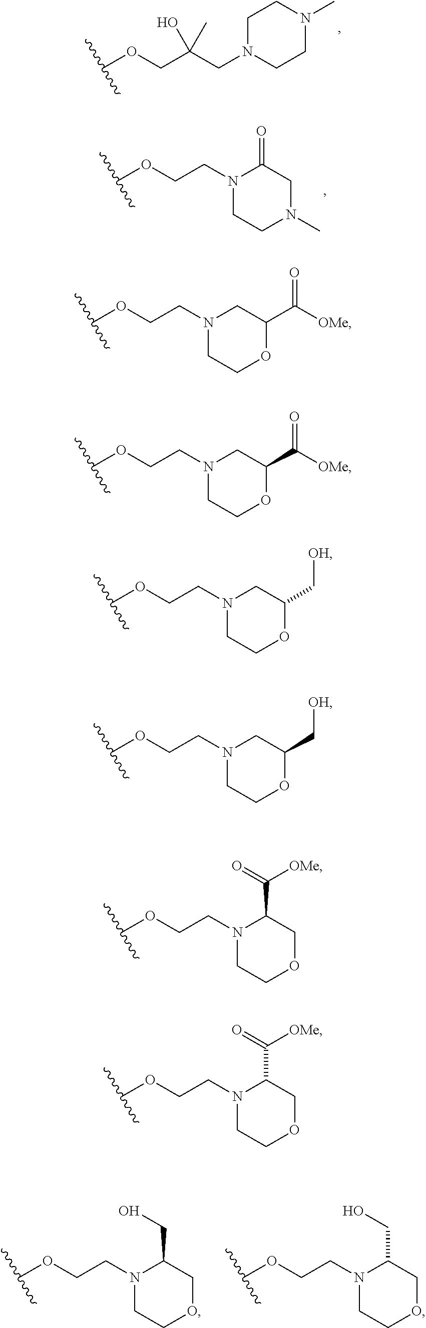

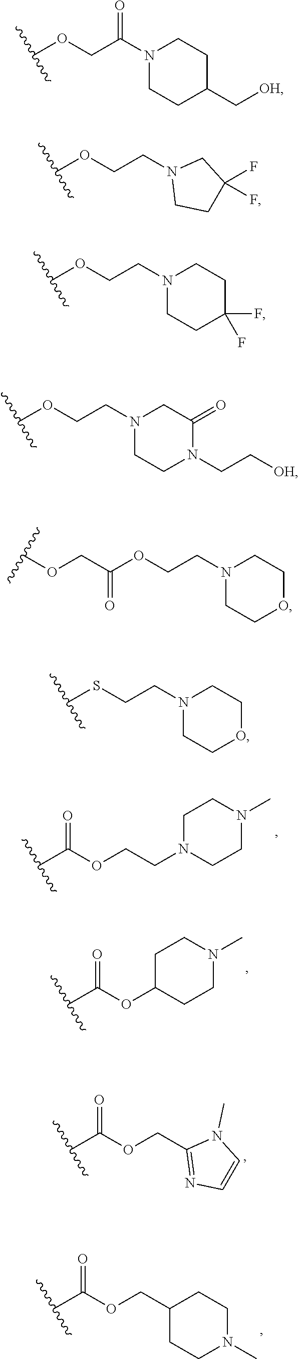

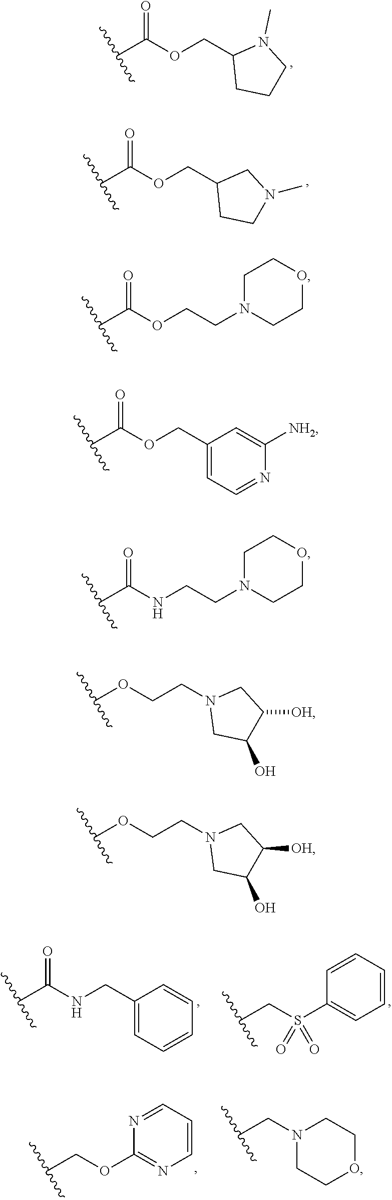

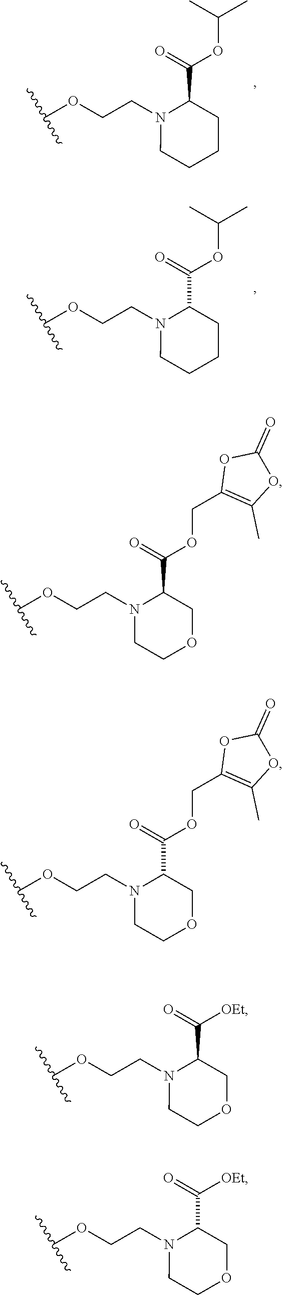

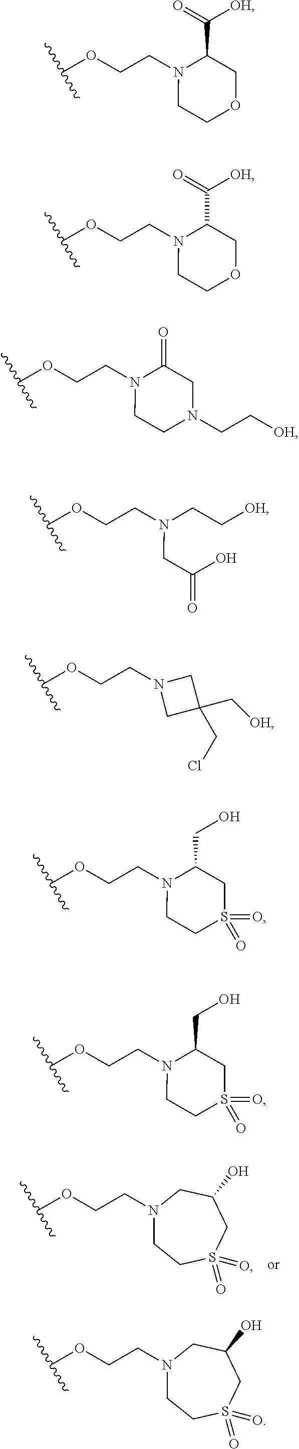

37. The compound of any one of paragraphs 1-36, wherein R^{2B} is hydrogen.
38. The compound of any one of paragraphs 1-36, wherein R^{2B} is optionally substituted C₁₋₆ alkyl or optionally substituted C₁₋₆ acyl.
39. The compound of any one of paragraphs 1-36, wherein R^{2B} is unsubstituted C₁₋₆ alkyl or unsubstituted C₁₋₆ acyl.
40. The compound of any one of paragraphs 1-38, wherein R^{2B} is one of the following: methyl, —CH₂OH, —CH₂OCH₂Ph, —CH₂O(C═O)Ph, —CH₂CO₂Me, —CO₂H, —CO₂Me, —CO₂CH₂Ph; or is of one of the following formulae:

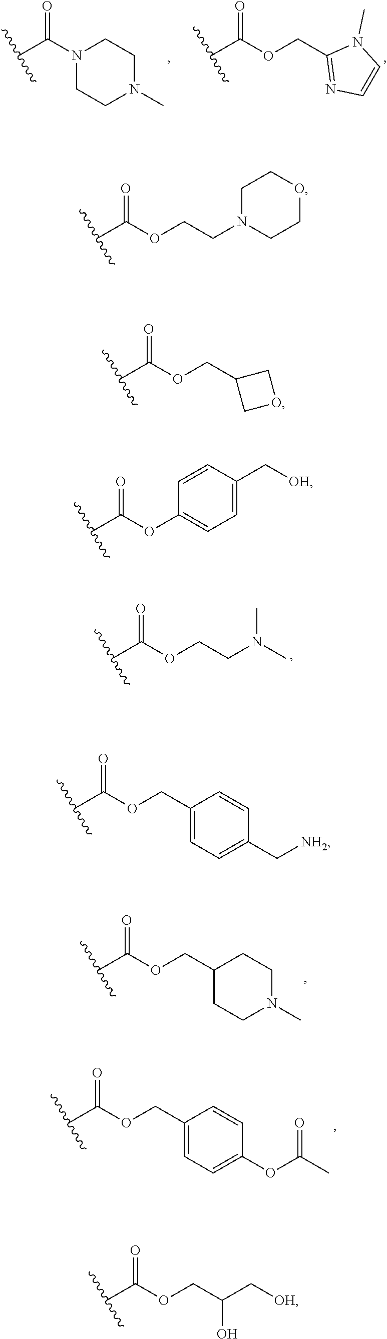

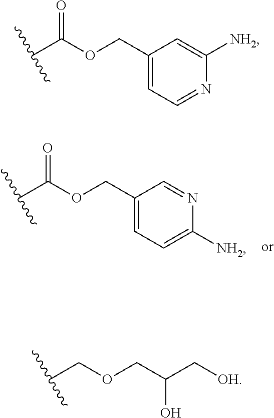 [SMILES: *C(=O)OCc1ccnc(N)c1 ; *C(=O)OCc1ccc(N)nc1 ; "or" ; *COCC(O)CO]

41. The compound of any one of paragraphs 1-40, wherein R^{N2} is hydrogen.
42. The compound of any one of paragraphs 1-40, wherein R^{N2} is optionally substituted C₁₋₆ alkyl.
43. The compound of any one of paragraphs 1-40, wherein R^{N2} is optionally substituted C₁₋₃ alkyl.
44. The compound of any one of paragraphs 1-40, wherein R^{N2} is unsubstituted C₁₋₃ alkyl.
45. The compound of any one of paragraphs 1-40, wherein R^{N2} is methyl or ethyl.
46. The compound of any one of paragraphs 1-40, wherein R^{N2} is dihalo- or trihalomethyl.
47. The compound of any one of paragraphs 1-40, wherein R^{N2} is —CHF₂ or —CF₃.
48. The compound of any one of paragraphs 1-47, wherein both R^{N1} are hydrogen.
49. The compound of paragraph 1, wherein the compound is selected from the compounds in Table A, and pharmaceutically acceptable salts thereof.
50. The compound of any one of the preceding paragraphs, or a pharmaceutically acceptable salt thereof, wherein the compound has an off-rate (T_{1/2}) of greater than 20 seconds from a soluble adenylyl cyclase (sAC) protein.
51. The compound of any one of the preceding paragraphs, or a pharmaceutically acceptable salt thereof, wherein the compound has an off-rate (T_{1/2}) of greater than 1,000 seconds from a sAC protein.
52. The compound of any one of the preceding paragraphs, or a pharmaceutically acceptable salt thereof, wherein the compound has an off-rate (T_{1/2}) of greater than 10,000 seconds from a sAC protein.
53. The compound of any one of the preceding paragraphs, or a pharmaceutically acceptable salt thereof, wherein the compound has an off-rate (T_{1/2}) of from 25-20,000 seconds from a sAC protein.
54. The compound of any one of the preceding paragraphs, or a pharmaceutically acceptable salt thereof, wherein the compound has an off-rate (T_{1/2}) of from 1,000-20,000 seconds from a sAC protein.
55. A pharmaceutical composition comprising a compound of any one of paragraphs 1-54, or a pharmaceutically acceptable salt thereof, and a pharmaceutically acceptable carrier or excipient.
56. A method for contraception, the method comprising administering to a subject a compound of any one of paragraphs 1-54, or a pharmaceutically acceptable salt thereof, or a pharmaceutical composition of paragraph 55.
57. The method of paragraph 56, wherein the method is a method for male contraception; and the subject is a male subject.
58. The method of paragraph 57, wherein the compound, or pharmaceutically acceptable salt thereof, or pharmaceutical composition thereof, is administered orally to the male subject.
59. The method of paragraph 56, wherein the method is a method for female contraception; and the subject is a female subject.
60. The method of paragraph 59, wherein the compound, or pharmaceutically acceptable salt thereof, or pharmaceutical composition thereof, is administered intravaginally to the female subject.
61. The method of paragraph 59, wherein the compound, or pharmaceutically acceptable salt thereof, or pharmaceutical composition thereof, is administered orally to the female subject.
62. A method for treating an ocular condition in a subject, the method comprising administering to the subject a compound of any one of paragraphs 1-54, or a pharmaceutically acceptable salt thereof, or a pharmaceutical composition of paragraph 55.
63. The method of paragraph 62, wherein the ocular condition is ocular hypotony.
64. A method for increasing intraocular pressure (IOP) in a subject, the method comprising administering to the subject a compound of any one of paragraphs 1-54, or a pharmaceutically acceptable salt thereof, or a pharmaceutical composition of paragraph 55.
65. A method for treating and/or preventing a liver disease in a subject, the method comprising administering to the subject a compound of any one of paragraphs 1-54, or a pharmaceutically acceptable salt thereof, or a pharmaceutical composition of paragraph 55.
66. The method of paragraph 65, wherein the liver disease is non-alcoholic steatohepatitis (NASH).
67. The method of paragraph 65, wherein the method is a method of preventing the development of NASH in a subject.
68. The method of paragraph 65, wherein the method is a method of preventing the worsening or progression of NASH in a subject.
69. A method for treating psoriasis in a subject, the method comprising administering to the subject a compound of any one of paragraphs 1-54, or a pharmaceutically acceptable salt thereof, or a pharmaceutical composition of paragraph 55.
70. A method for treating an inflammatory or autoimmune disease in a subject, the method comprising administering to the subject a compound of any one of paragraphs 1-54, or a pharmaceutically acceptable salt thereof, or a pharmaceutical composition of paragraph 55.
71. The method of paragraph 70, wherein the inflammatory or autoimmune disease is a Th17-mediated inflammatory or autoimmune disease.
72. The method of paragraph 70, wherein the inflammatory or autoimmune disease is a type 17 inflammatory or autoimmune disease.
73. A method for treating a disease in a subject, the method comprising administering to the subject a compound of any one of paragraphs 1-54, or a pharmaceutically acceptable salt thereof, or a pharmaceutical composition of paragraph 55.
74. The method of paragraph 73, wherein the disease is typically associated with the activity of a sAC enzyme.
75. A method for inhibiting the activity of soluble adenylyl cyclase (sAC) in a subject or biological sample, the method comprising administering to the subject or contacting the biological sample with a compound of any one of paragraphs 1-54, or a pharmaceutically acceptable salt thereof, or a pharmaceutical composition of paragraph 55.
76. The method of any one of paragraphs 1-75, wherein the subject is a human.
77. The method of any one of paragraphs 1-75, wherein the subject is a non-human mammal.
78. The method of any one of paragraphs 1-75, wherein the subject is a canine.
79. The method of paragraph 75, wherein the inhibiting occurs in vivo in a subject.
80. The method of paragraph 75, wherein the inhibiting occurs in vitro.
81. A compound of any one of paragraphs 1-54, or a pharmaceutically acceptable salt thereof, or a pharmaceutical composition of paragraph 55, for use in treating a disease in a subject.
82. Use of a compound of any one of paragraphs 1-54, or a pharmaceutically acceptable salt thereof, or a pharmaceutical composition of paragraph 555, for the manufacture of a medicament for treating a disease in a subject.
83. A method for male contraception comprising administering to a male subject a soluble adenylyl cyclase (sAC) inhibitor with an off-rate (T_{1/2}) of greater than 20 seconds from a sAC protein.
84. The method of paragraph 83, wherein the sAC inhibitor has an off-rate (T_{1/2}) of greater than 1,000 seconds from a sAC protein.
85. The method of paragraph 83, wherein the sAC inhibitor has an off-rate (T_{1/2}) of greater than 10,000 seconds from a sAC protein.
86. The method of paragraph 83, wherein the sAC inhibitor has an off-rate (T_{1/2}) of from 25-20,000 seconds from a sAC protein.
87. The method of paragraph 83, wherein the sAC inhibitor has an off-rate (T_{1/2}) of from 1,000-20,000 seconds from a sAC protein.
88. A kit comprising:
(i) an oral contraceptive pill for administration to a male comprising a compound of any one of the preceding paragraphs, or a pharmaceutically acceptable salt thereof, or a pharmaceutical composition thereof; and
(ii) an oral contraceptive pill for administration to a female comprising a compound of any one of the preceding paragraphs, or a pharmaceutically acceptable salt thereof, or a pharmaceutical composition thereof; and optionally instructions for use.

What is claimed is:

1. A compound of Formula (I): 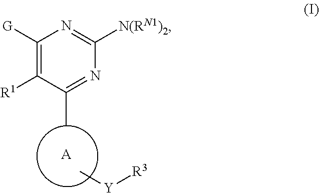
or a pharmaceutically acceptable salt thereof, wherein:
G is halogen, —CN, optionally substituted alkyl, or optionally substituted acyl;
R¹ is hydrogen, halogen, optionally substituted alkyl, or optionally substituted acyl;
A is an optionally substituted monocyclic heteroaryl ring comprising at least 1 nitrogen atom;
Y is a bond, optionally substituted alkylene, optionally substituted heteroalkylene, —O—, —NR^{N}—, —S(═O)—, or —SO₂—;
R³ is optionally substituted carbocyclyl, optionally substituted heterocyclyl, optionally substituted aryl, or optionally substituted heteroaryl;
each instance of R^{N1} is independently hydrogen, optionally substituted alkyl, optionally substituted acyl, or a nitrogen protecting group, or optionally two R^{N1} are taken together with the intervening atoms to form optionally substituted heterocyclyl or optionally substituted heteroaryl;
provided that when G is not halogen, -(A)-Y—R³ is of the formula: 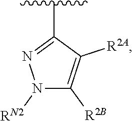
wherein:
R^{2A} and R^{2B} are independently hydrogen, halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, —SR^{S}, or —Y—R³;
provided that one of R^{2A} and R^{2B} is —Y—R³;
R^{N2} is hydrogen, optionally substituted alkyl, optionally substituted acyl, or a nitrogen protecting group;
each instance of R^{N} is independently hydrogen, optionally substituted alkyl, optionally substituted acyl, or a nitrogen protecting group, or optionally two R^{N} are taken together with the intervening atoms to form optionally substituted heterocyclyl or optionally substituted heteroaryl;
each instance of R^{O} is independently hydrogen, optionally substituted alkyl, optionally substituted acyl, or an oxygen protecting group; and
each instance of R^{S} is independently hydrogen, optionally substituted alkyl, optionally substituted acyl, or a sulfur protecting group.

2. The compound of claim 1, wherein A is an optionally substituted 5-membered heteroaryl ring comprising 2 or 3 nitrogen atoms.

3. The compound of claim 1 or 2, wherein A is an optionally substituted pyrazole ring.

4. The compound of any one f claims 1-3 wherein the compound is of Formula (II): 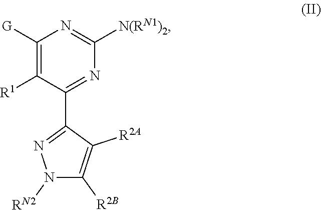
or a pharmaceutically acceptable salt thereof, wherein one of R^{2A} and R^{2B} is —Y—R³.

5. The compound of any one of claims 1-4, wherein G is halogen.

6. The compound of any one of claims 1-5, wherein G is —Cl.

7. The compound of any one of claims 1-6, wherein R¹ is hydrogen.

8. The compound of any one of claims 1-7, wherein the compound is of Formula (III): 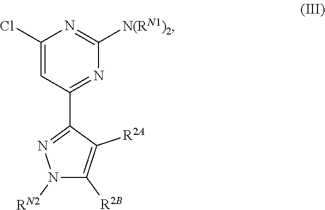
or a pharmaceutically acceptable salt thereof, wherein one of R^{2A} and R^{2B} is —Y—R³.

9. The compound of any one of claims 1-8, wherein the compound is of Formula (IV): 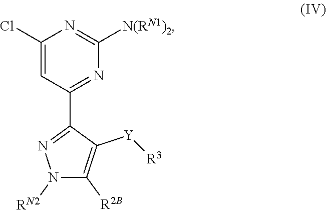
or a pharmaceutically acceptable salt thereof.

10. The compound of any one of claims 1-9, wherein R³ is optionally substituted phenyl.

11. The compound of any one of claims 1-10, wherein the compound is of Formula (V): 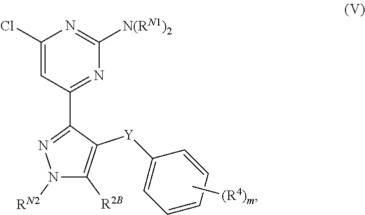
or a pharmaceutically acceptable salt thereof, wherein:
each instance of R⁴ is independently halogen, —CN, —N₃, —NO₂, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, —OR^{O}, —N(R^{N})₂, or —SR^{S}; and
m is 0, 1, 2, 3, 4, or 5.

12. The compound of any one of claims 1-11, wherein Y is optionally substituted C₁₋₃ alkylene.

13. The compound of any one of claims 1-12, wherein Y is optionally substituted methylene.

14. The compound of any one of claims 1-12, the compound is of Formula (VI): 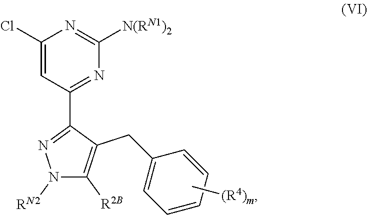
or a pharmaceutically acceptable salt thereof.

15. The compound of any one of claims 1-14, wherein at least one instance of R^{N1} is hydrogen.

16. The compound of any one of claims 1-15, wherein the compound is of the formula: 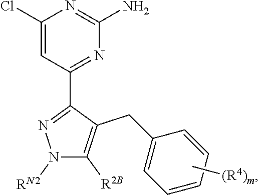
or a pharmaceutically acceptable salt thereof.

17. The compound of any one of claims 11-16, wherein m is 1.

18. The compound of any one of claims 1-17, wherein the compound is of the formula: 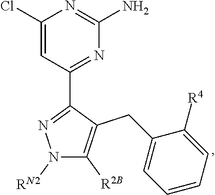
or a pharmaceutically acceptable salt thereof.

19. The compound of any one of claims 11-18, wherein at least one instance of R⁴ is halogen.

20. The compound of claim 19, wherein at least one instance of R⁴ is —Cl or —F.

21. The compound of any one of claims 11-20, wherein at least one instance of R⁴ is optionally substituted C₁₋₆ alkyl or optionally substituted C₁₋₆ acyl.

22. The compound of claim 21, wherein at least one instance of R⁴ is one of the following: —CO₂H, —CO₂Me, —CO₂CH₂Ph, —CH₂OCH₂CH₂NMe₂, —C(═O)NHCH₂Ph, —C(═O)NHMe, —C(═O)NHCH₂CH₂OMe, or —CO₂CH₂CH₂CH₂NMe₂; or is of the following formula:

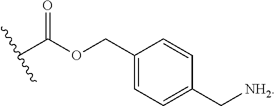 [SMILES: *C(=O)OCc1ccc(CN)cc1]

23. The compound of any one of claims 11-22, wherein at least one instance of R⁴ is optionally substituted aryl or optionally substituted carbocyclyl.

24. The compound of claim 23, wherein at least one instance of R⁴ is of one of the following formulae:

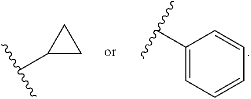

25. The compound of any one of claims 11-24, wherein at least one instance of R⁴ is —OR^{O}.

26. The compound of claim 25, wherein at least one instance of R⁴ is one of the following: —OMe, —OCF₃, —OCH₂CO₂Me, —O(CH₂CH₂O)₃Me; or is of one of the following formulae:

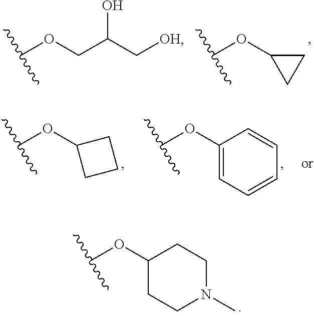

27. The compound of any one of claims 11-26, wherein at least one instance of R⁴ is —Z—R⁵; wherein Z is a bond, optionally substituted alkylene, optionally substituted heteroalkylene, or optionally substituted acylene; and R⁵ optionally substituted heterocyclyl, optionally substituted heteroaryl, —N(R^{N})₂, or —OR^{O}.

28. The compound of claim 27, wherein Z is optionally substituted C₁₋₆ alkylene, optionally substituted C₁₋₆ heteroalkylene, or optionally substituted C₁₋₆ acylene.

29. The compound of claim 27 or 28, wherein Z is optionally substituted C₁₋₆ heteroalkylene.

30. The compound of claim 27 or 28, wherein Z is optionally substituted C₁₋₃ heteroalkylene.

31. The compound of claim 27 or 28, wherein Z is unsubstituted C₁₋₃ heteroalkylene.

32. The compound of claim 27 or 28, wherein Z is of one of the following formulae:

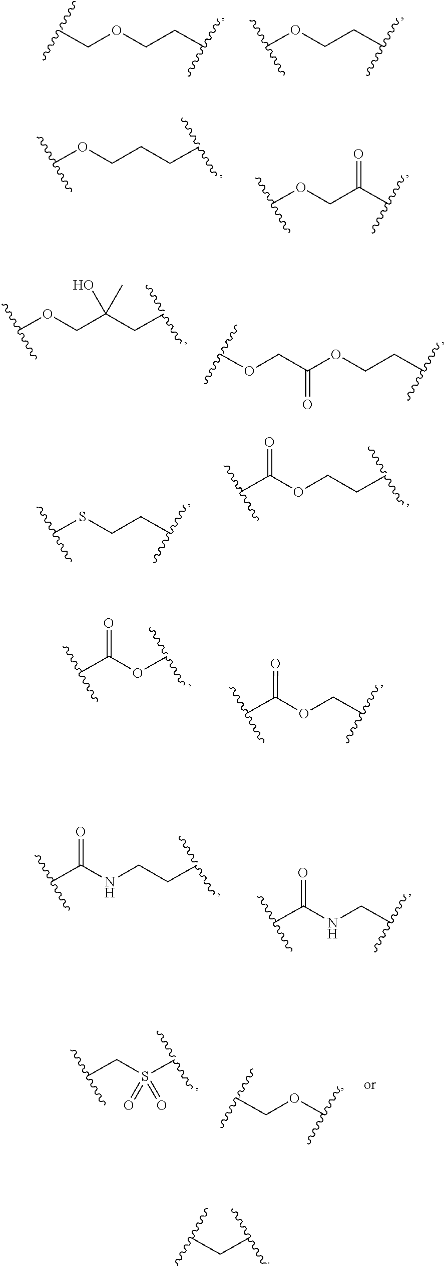

33. The compound of any one of claims 27-32, wherein R⁵ is optionally substituted 4- to 7-membered heterocyclyl.

34. The compound of any one of claims 27-34, wherein R⁵ is optionally substituted 5- or 6-membered heterocyclyl comprising 1 or 2 heteroatoms independently selected from N and O.

35. The compound of any one of claims 27-32, wherein R⁵ is of one of the following formulae:

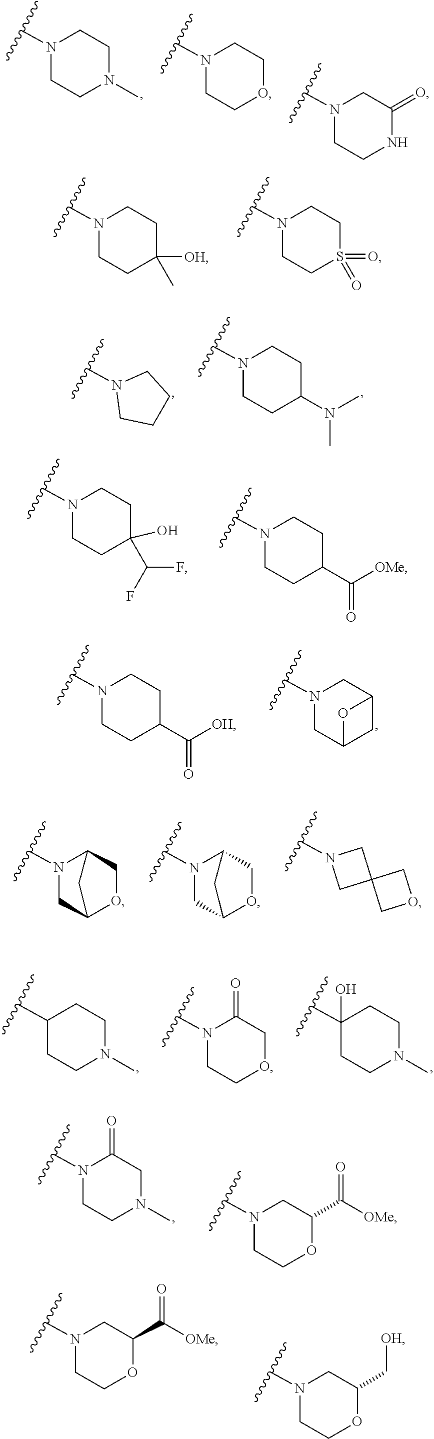

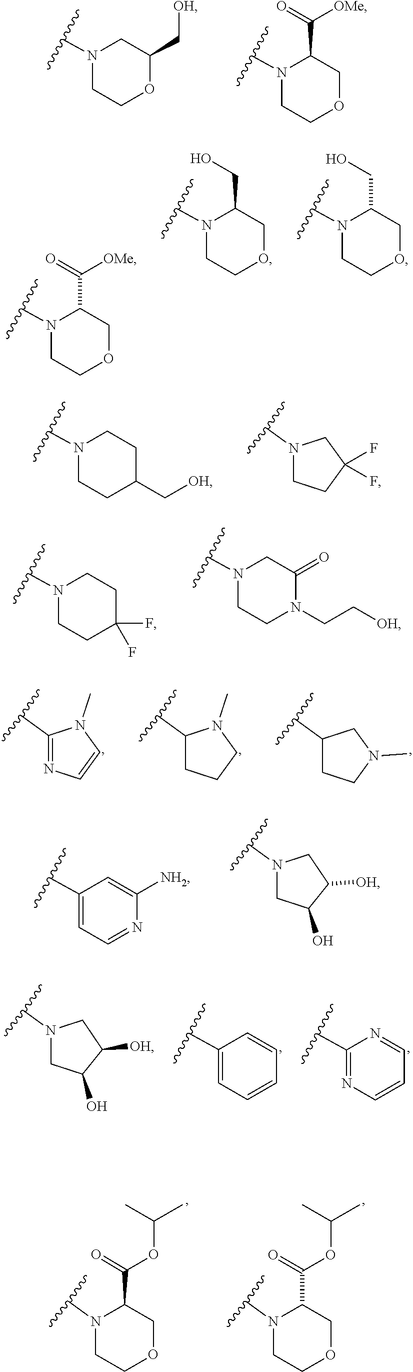

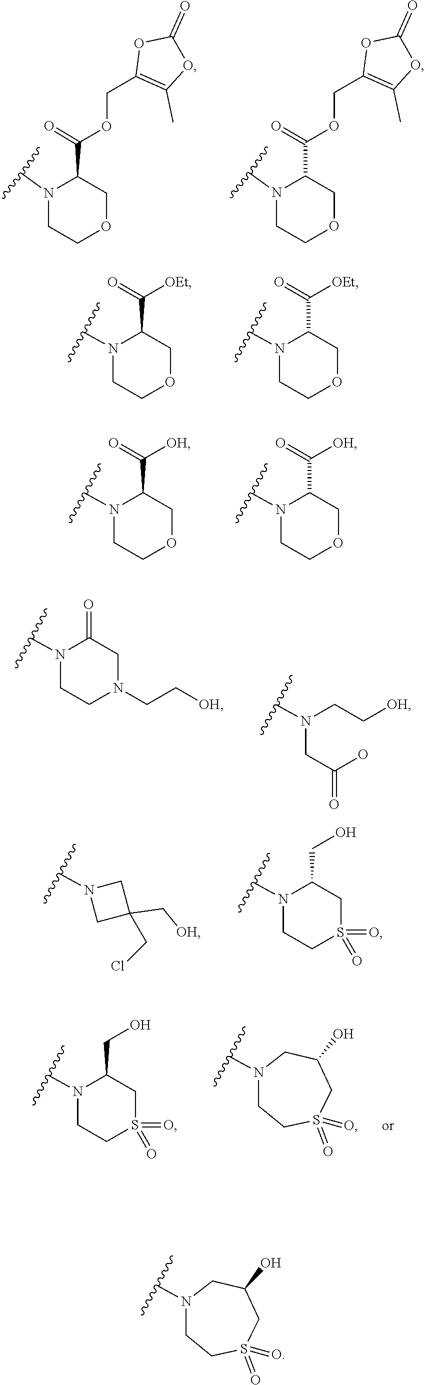

36. The compound of claim 27, wherein at least one instance of R⁴ is of one of the following formulae:

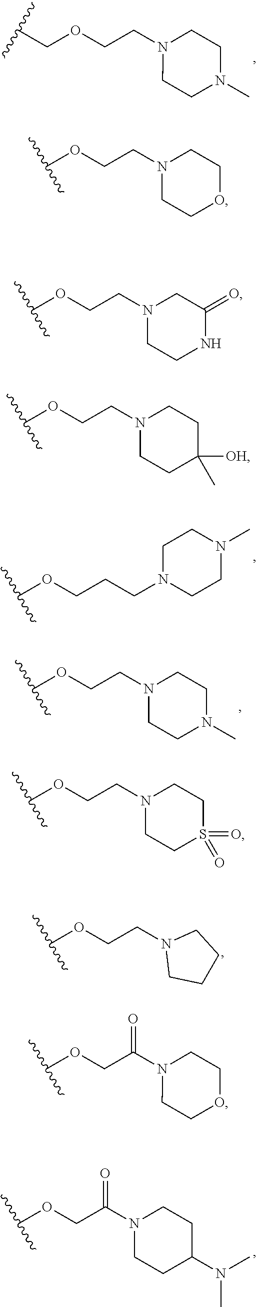

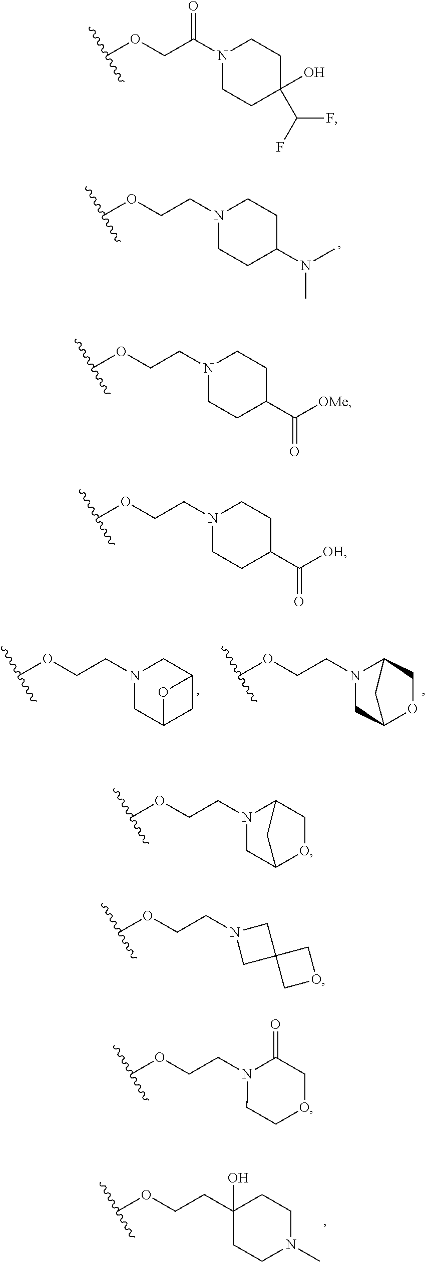

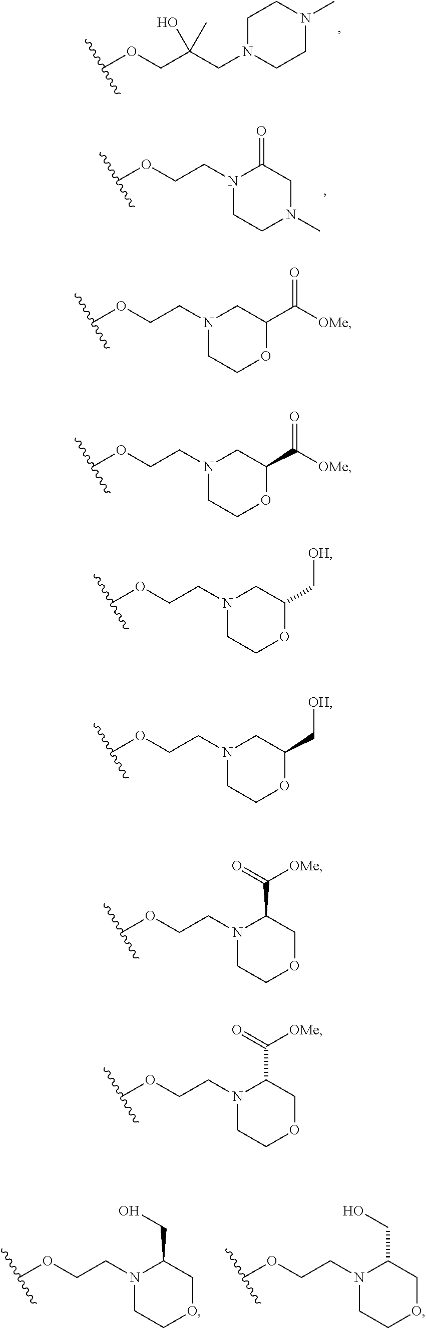

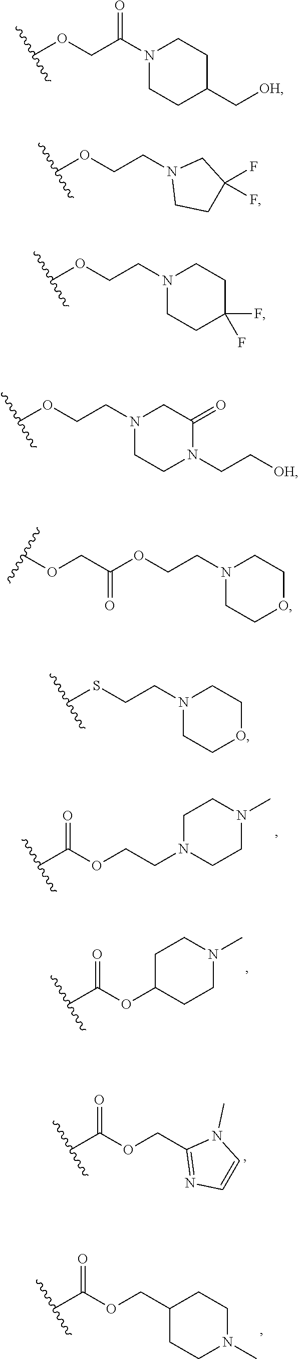

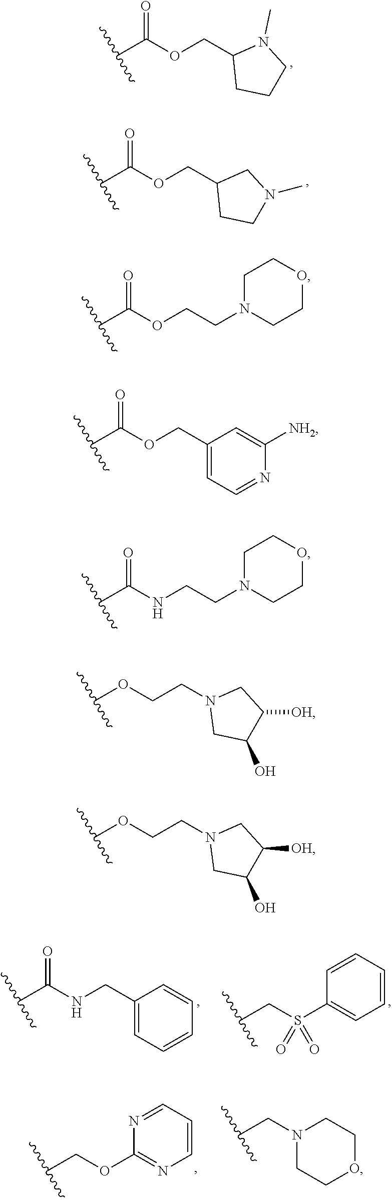

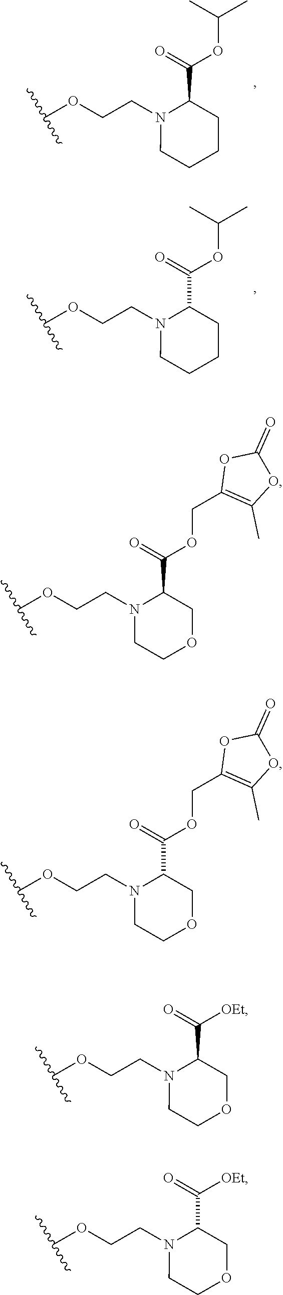

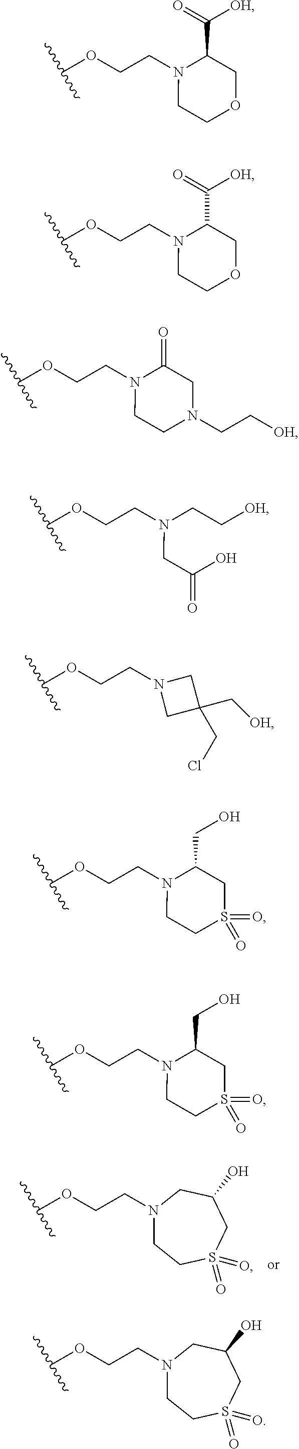

37. The compound of any one of claims 1-36, wherein R^{2B} is hydrogen.

38. The compound of any one of claims 1-36, wherein R^{2B} is optionally substituted C₁₋₆ alkyl or optionally substituted C₁₋₆ acyl.

39. The compound of any one of claims 1-36, wherein R^{2B} is unsubstituted C₁₋₆ alkyl or unsubstituted C₁₋₆ acyl.

40. The compound of any one of claims 1-38, wherein R^{2B} is one of the following: methyl, —CH₂OH, —CH₂OCH₂Ph, —CH₂O(C═O)Ph, —CH₂CO₂Me, —CO₂H, —CO₂Me, —CO₂CH₂Ph; or is of one of the following formulae:

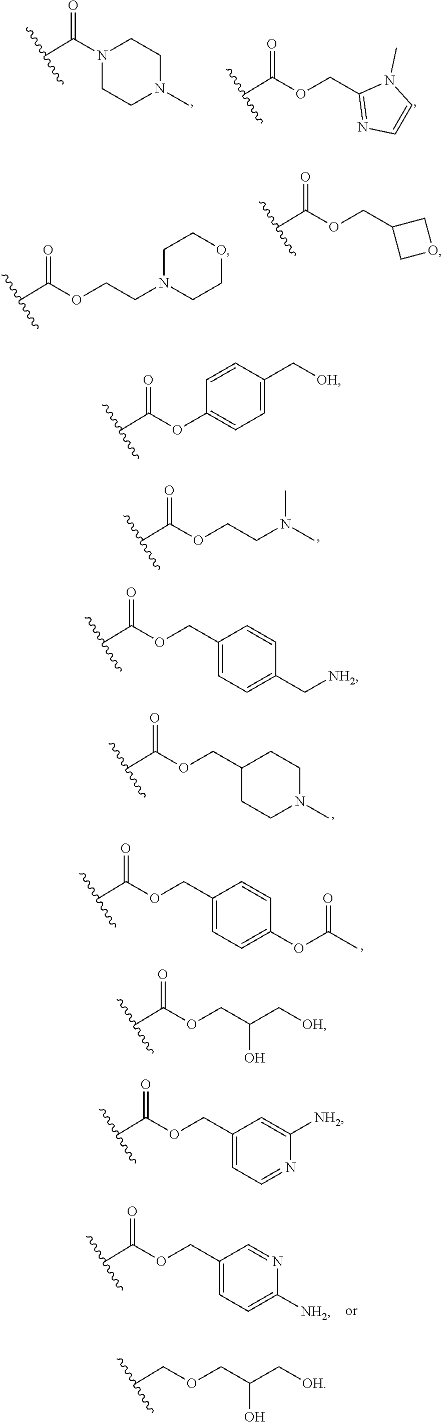

41. The compound of any one of claims 1-40, wherein R^{N2} is hydrogen.

42. The compound of any one of claims 1-40, wherein R^{N2} is optionally substituted C₁₋₆ alkyl.

43. The compound of any one of claims 1-40, wherein R^{N2} is optionally substituted C₁₋₃ alkyl.

44. The compound of any one of claims 1-40, wherein R^{N2} is unsubstituted C₁₋₃ alkyl.

45. The compound of any one of claims 1-40, wherein R^{N2} is methyl or ethyl.

46. The compound of any one of claims 1-40, wherein R^{N2} is dihalo- or trihalomethyl.

47. The compound of any one of claims 1-40, wherein R^{N2} is —CHF₂ or —CF₃.

48. The compound of any one of claims 1-47, wherein both R^{N1} are hydrogen.

49. The compound of claim 1, wherein the compound is selected from the group consisting of: 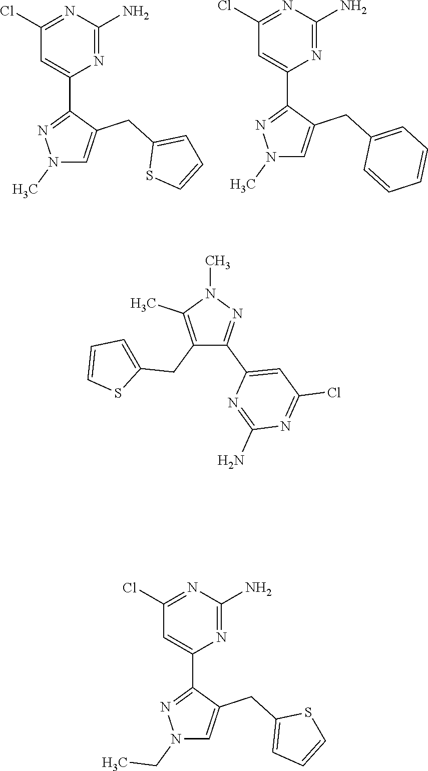 [SMILES: Cn1cc(Cc2cccs2)c(-c2cc(Cl)nc(N)n2)n1 ; Cn1cc(Cc2ccccc2)c(-c2cc(Cl)nc(N)n2)n1 ; Cc1c(Cc2cccs2)c(-c2cc(Cl)nc(N)n2)nn1C ; CCn1cc(Cc2cccs2)c(-c2cc(Cl)nc(N)n2)n1] 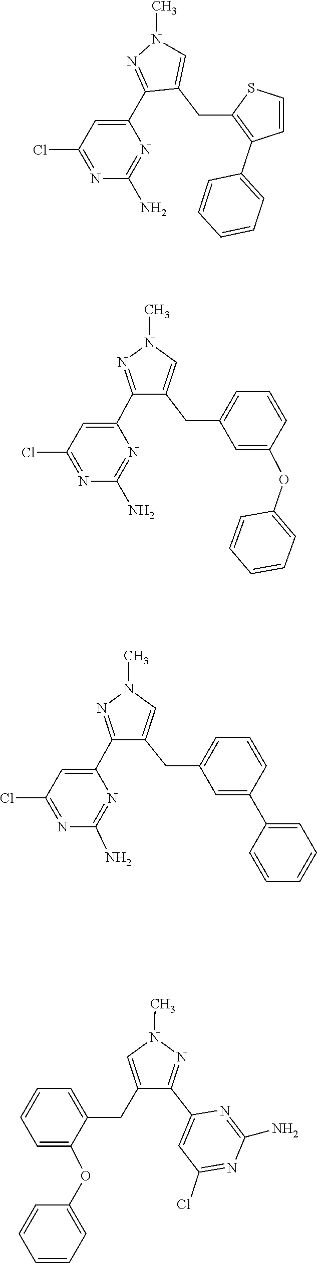 [SMILES: Cn1cc(Cc2sccc2-c2ccccc2)c(-c2cc(Cl)nc(N)n2)n1 ; Cn1cc(Cc2cccc(Oc3ccccc3)c2)c(-c2cc(Cl)nc(N)n2)n1 ; Cn1cc(Cc2cccc(-c3ccccc3)c2)c(-c2cc(Cl)nc(N)n2)n1 ; Cn1cc(Cc2ccccc2Oc2ccccc2)c(-c2cc(Cl)nc(N)n2)n1] 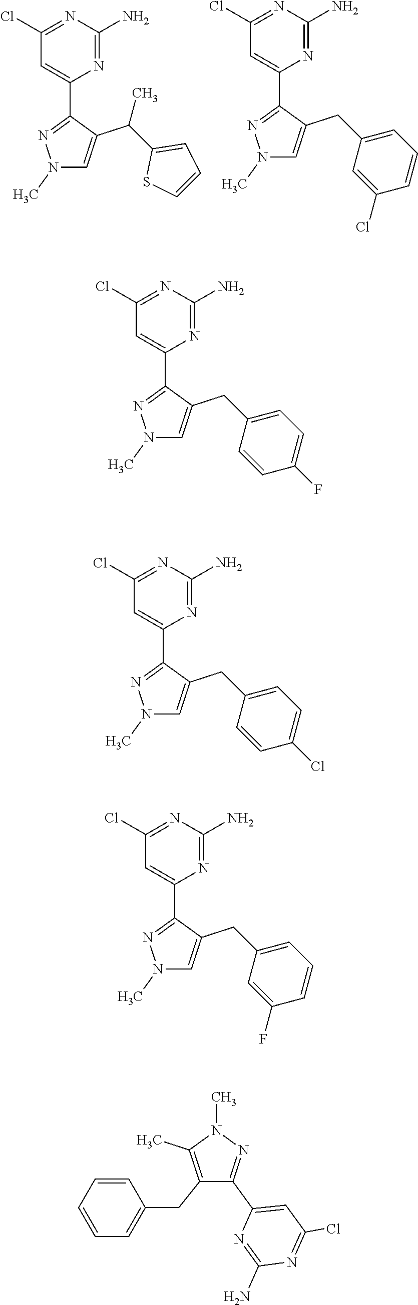 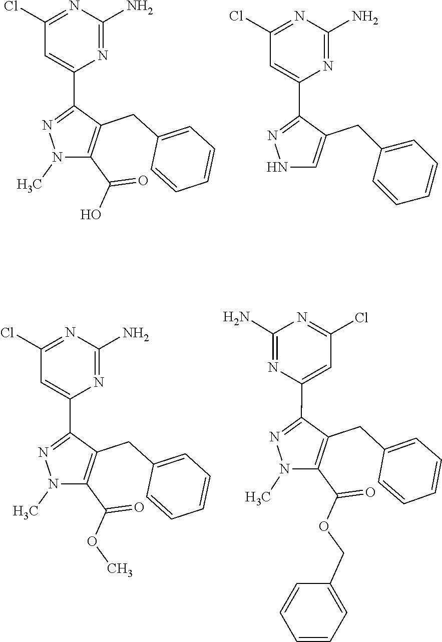 [SMILES: Cn1nc(-c2cc(Cl)nc(N)n2)c(Cc2ccccc2)c1C(=O)O ; Nc1nc(Cl)cc(-c2n[nH]cc2Cc2ccccc2)n1 ; COC(=O)c1c(Cc2ccccc2)c(-c2cc(Cl)nc(N)n2)nn1C ; Cn1nc(-c2cc(Cl)nc(N)n2)c(Cc2ccccc2)c1C(=O)OCc1ccccc1] 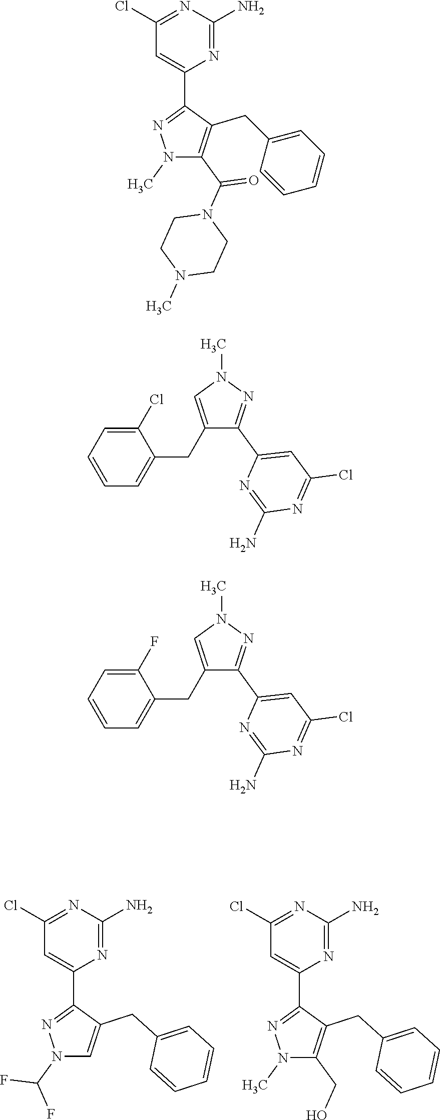 [SMILES: CN1CCN(C(=O)c2c(Cc3ccccc3)c(-c3cc(Cl)nc(N)n3)nn2C)CC1 ; Cn1cc(Cc2ccccc2Cl)c(-c2cc(Cl)nc(N)n2)n1 ; Cn1cc(Cc2ccccc2F)c(-c2cc(Cl)nc(N)n2)n1 ; Nc1nc(Cl)cc(-c2nn(C(F)F)cc2Cc2ccccc2)n1 ; Cn1nc(-c2cc(Cl)nc(N)n2)c(Cc2ccccc2)c1CO] 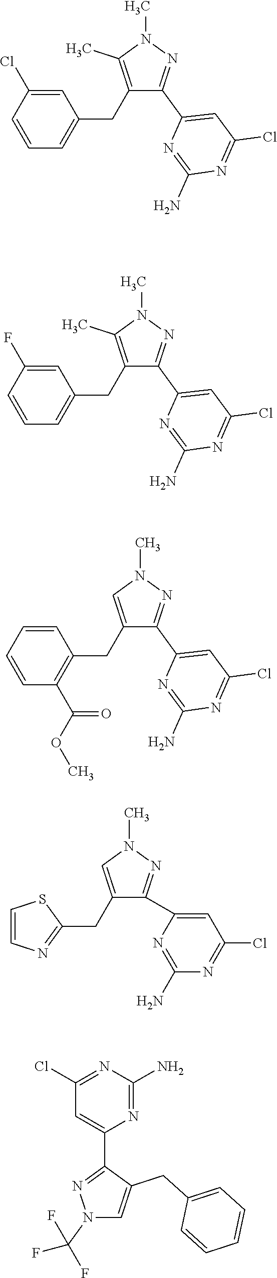 [SMILES: Cc1c(Cc2cccc(Cl)c2)c(-c2cc(Cl)nc(N)n2)nn1C ; Cc1c(Cc2cccc(F)c2)c(-c2cc(Cl)nc(N)n2)nn1C ; COC(=O)c1ccccc1Cc1cn(C)nc1-c1cc(Cl)nc(N)n1 ; Cn1cc(Cc2nccs2)c(-c2cc(Cl)nc(N)n2)n1 ; Nc1nc(Cl)cc(-c2nn(C(F)(F)F)cc2Cc2ccccc2)n1] 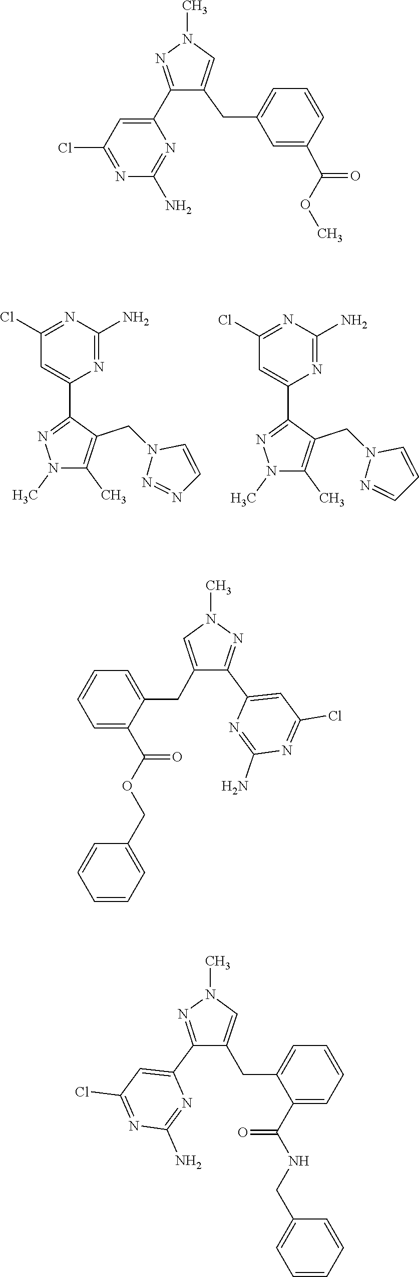 [SMILES: COC(=O)c1cccc(Cc2cn(C)nc2-c2cc(Cl)nc(N)n2)c1 ; Cc1c(Cn2ccnn2)c(-c2cc(Cl)nc(N)n2)nn1C ; Cc1c(Cn2cccn2)c(-c2cc(Cl)nc(N)n2)nn1C ; Cn1cc(Cc2ccccc2C(=O)OCc2ccccc2)c(-c2cc(Cl)nc(N)n2)n1 ; Cn1cc(Cc2ccccc2C(=O)NCc2ccccc2)c(-c2cc(Cl)nc(N)n2)n1] 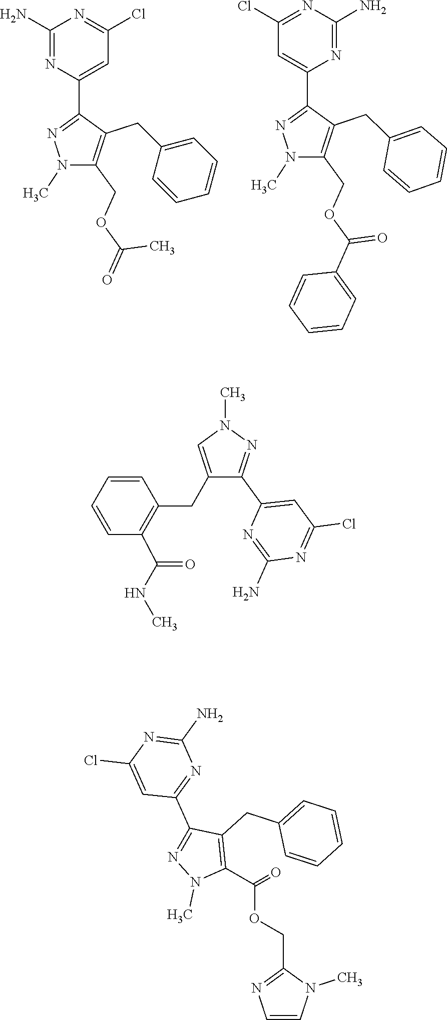 [SMILES: CC(=O)OCc1c(Cc2ccccc2)c(-c2cc(Cl)nc(N)n2)nn1C ; Cn1nc(-c2cc(Cl)nc(N)n2)c(Cc2ccccc2)c1COC(=O)c1ccccc1 ; CNC(=O)c1ccccc1Cc1cn(C)nc1-c1cc(Cl)nc(N)n1 ; Cn1ccnc1COC(=O)c1c(Cc2ccccc2)c(-c2cc(Cl)nc(N)n2)nn1C] 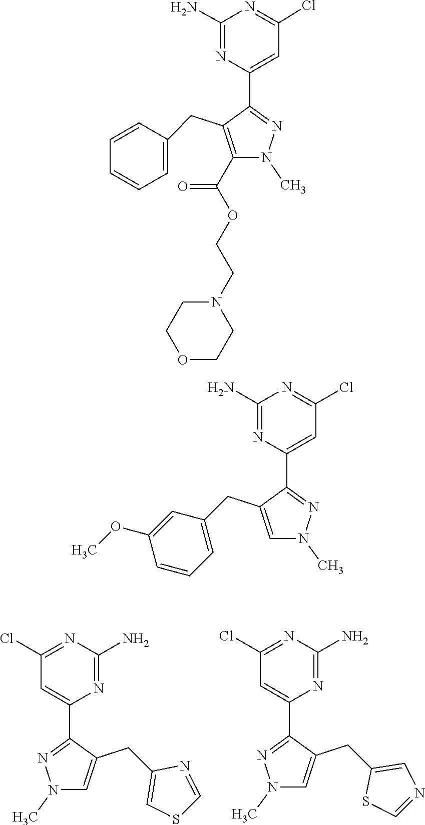 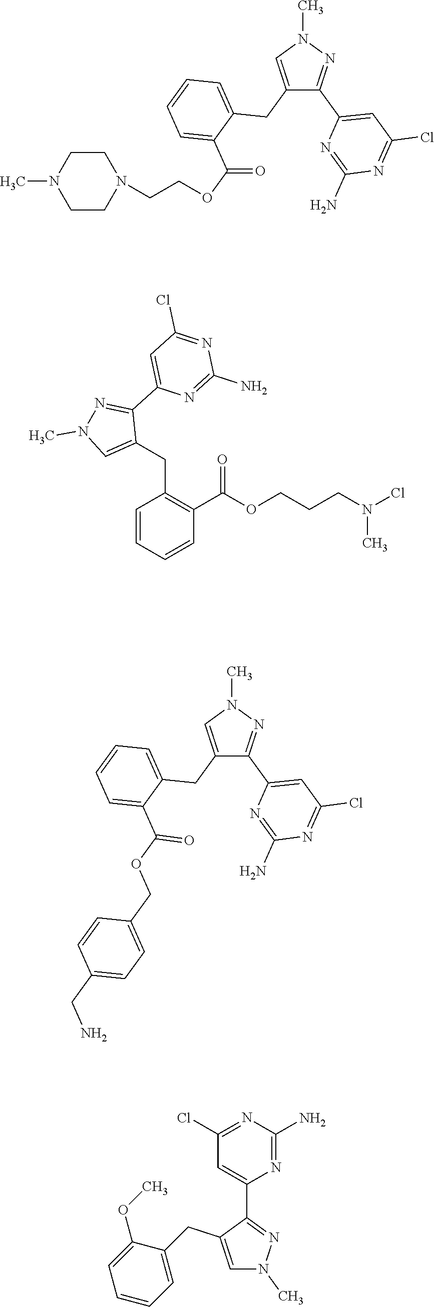 [SMILES: CN1CCN(CCOC(=O)c2ccccc2Cc2cn(C)nc2-c2cc(Cl)nc(N)n2)CC1 ; CN(Cl)CCCOC(=O)c1ccccc1Cc1cn(C)nc1-c1cc(Cl)nc(N)n1 ; Cn1cc(Cc2ccccc2C(=O)OCc2ccc(CN)cc2)c(-c2cc(Cl)nc(N)n2)n1 ; COc1ccccc1Cc1cn(C)nc1-c1cc(Cl)nc(N)n1] 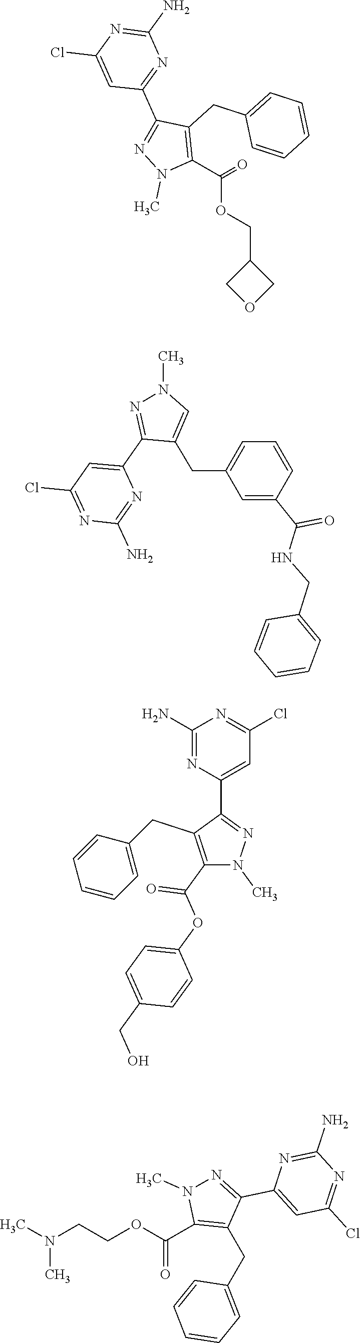 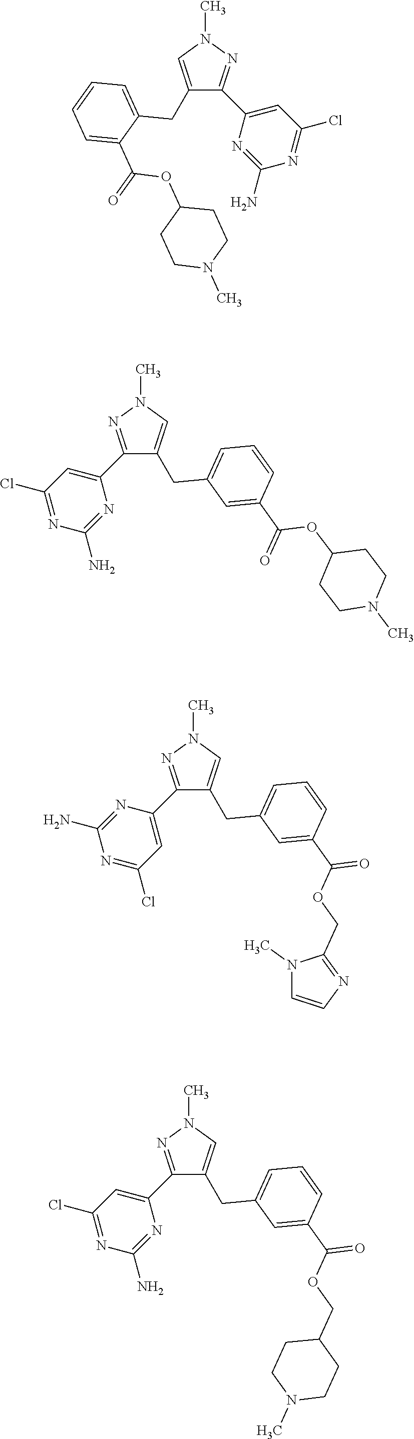 [SMILES: CN1CCC(OC(=O)c2ccccc2Cc2cn(C)nc2-c2cc(Cl)nc(N)n2)CC1 ; CN1CCC(OC(=O)c2cccc(Cc3cn(C)nc3-c3cc(Cl)nc(N)n3)c2)CC1 ; Cn1cc(Cc2cccc(C(=O)OCc3nccn3C)c2)c(-c2cc(Cl)nc(N)n2)n1 ; CN1CCC(COC(=O)c2cccc(Cc3cn(C)nc3-c3cc(Cl)nc(N)n3)c2)CC1] 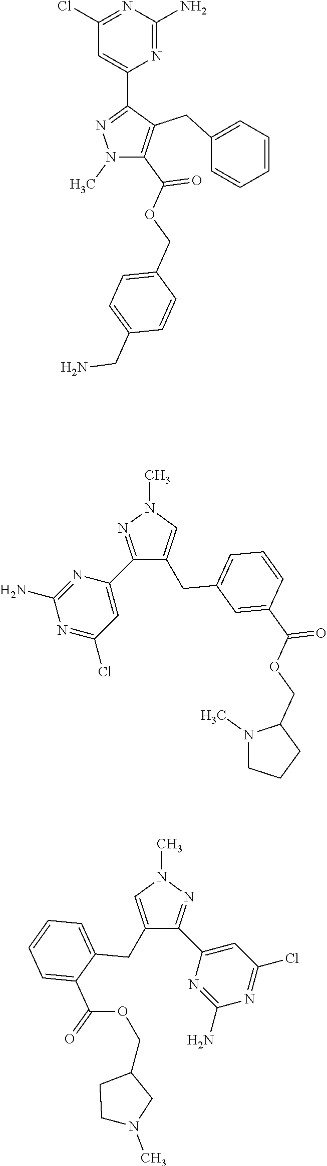 [SMILES: Cn1nc(-c2cc(Cl)nc(N)n2)c(Cc2ccccc2)c1C(=O)OCc1ccc(CN)cc1 ; CN1CCCC1COC(=O)c1cccc(Cc2cn(C)nc2-c2cc(Cl)nc(N)n2)c1 ; CN1CCC(COC(=O)c2ccccc2Cc2cn(C)nc2-c2cc(Cl)nc(N)n2)C1] 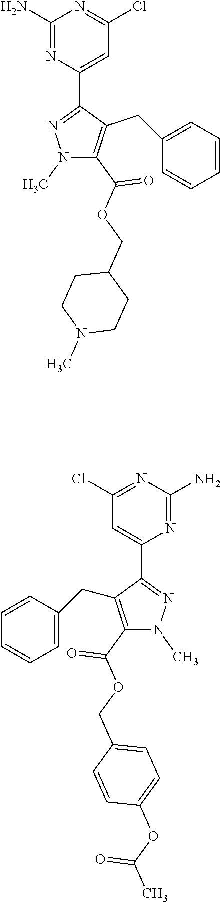 [SMILES: CN1CCC(COC(=O)c2c(Cc3ccccc3)c(-c3cc(Cl)nc(N)n3)nn2C)CC1 ; CC(=O)Oc1ccc(COC(=O)c2c(Cc3ccccc3)c(-c3cc(Cl)nc(N)n3)nn2C)cc1] 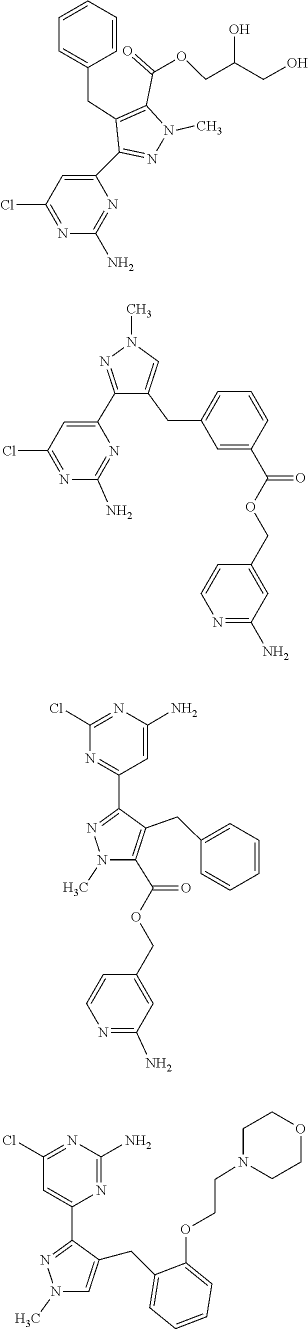 [SMILES: Cn1nc(-c2cc(Cl)nc(N)n2)c(Cc2ccccc2)c1C(=O)OCC(O)CO ; Cn1cc(Cc2cccc(C(=O)OCc3ccnc(N)c3)c2)c(-c2cc(Cl)nc(N)n2)n1 ; Cn1nc(-c2cc(N)nc(Cl)n2)c(Cc2ccccc2)c1C(=O)OCc1ccnc(N)c1 ; Cn1cc(Cc2ccccc2OCCN2CCOCC2)c(-c2cc(Cl)nc(N)n2)n1] 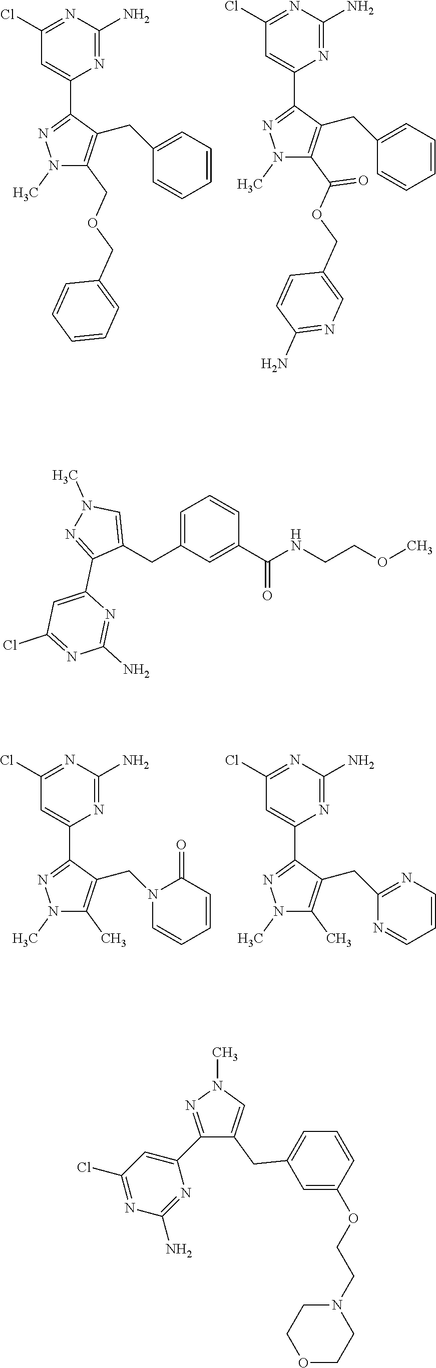 [SMILES: Cn1nc(-c2cc(Cl)nc(N)n2)c(Cc2ccccc2)c1COCc1ccccc1 ; Cn1nc(-c2cc(Cl)nc(N)n2)c(Cc2ccccc2)c1C(=O)OCc1ccc(N)nc1 ; COCCNC(=O)c1cccc(Cc2cn(C)nc2-c2cc(Cl)nc(N)n2)c1 ; Cc1c(Cn2ccccc2=O)c(-c2cc(Cl)nc(N)n2)nn1C ; Cc1c(Cc2ncccn2)c(-c2cc(Cl)nc(N)n2)nn1C ; Cn1cc(Cc2cccc(OCCN3CCOCC3)c2)c(-c2cc(Cl)nc(N)n2)n1] 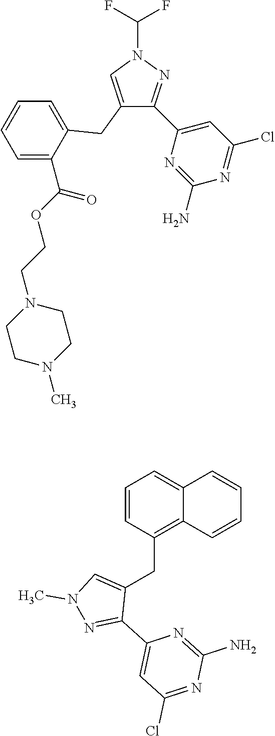 [SMILES: CN1CCN(CCOC(=O)c2ccccc2Cc2cn(C(F)F)nc2-c2cc(Cl)nc(N)n2)CC1 ; Cn1cc(Cc2cccc3ccccc23)c(-c2cc(Cl)nc(N)n2)n1] 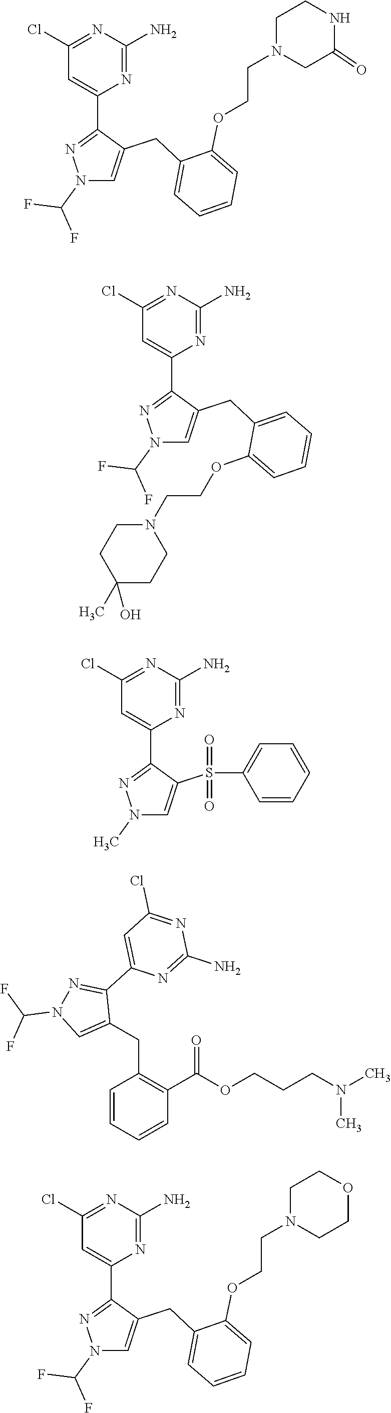 [SMILES: Nc1nc(Cl)cc(-c2nn(C(F)F)cc2Cc2ccccc2OCCN2CCNC(=O)C2)n1 ; CC1(O)CCN(CCOc2ccccc2Cc2cn(C(F)F)nc2-c2cc(Cl)nc(N)n2)CC1 ; Cn1cc(S(=O)(=O)c2ccccc2)c(-c2cc(Cl)nc(N)n2)n1 ; CN(C)CCCOC(=O)c1ccccc1Cc1cn(C(F)F)nc1-c1cc(Cl)nc(N)n1 ; Nc1nc(Cl)cc(-c2nn(C(F)F)cc2Cc2ccccc2OCCN2CCOCC2)n1] 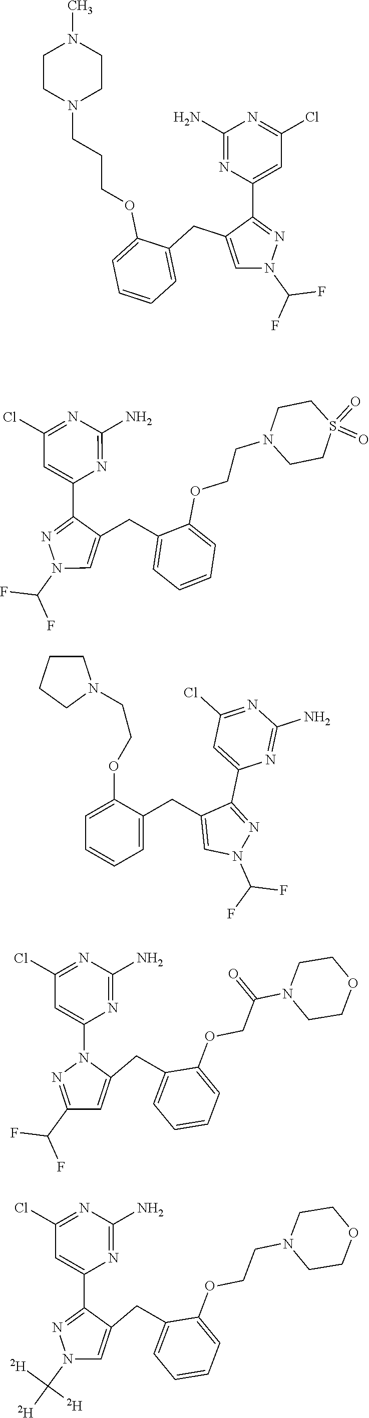 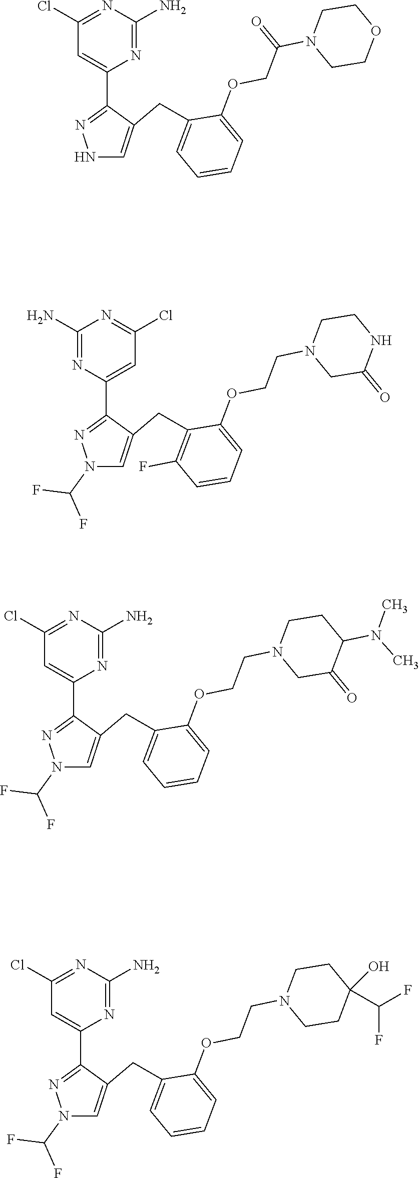 [SMILES: Nc1nc(Cl)cc(-c2n[nH]cc2Cc2ccccc2OCC(=O)N2CCOCC2)n1 ; Nc1nc(Cl)cc(-c2nn(C(F)F)cc2Cc2c(F)cccc2OCCN2CCNC(=O)C2)n1 ; CN(C)C1CCN(CCOc2ccccc2Cc2cn(C(F)F)nc2-c2cc(Cl)nc(N)n2)CC1=O ; Nc1nc(Cl)cc(-c2nn(C(F)F)cc2Cc2ccccc2OCCN2CCC(O)(C(F)F)CC2)n1] 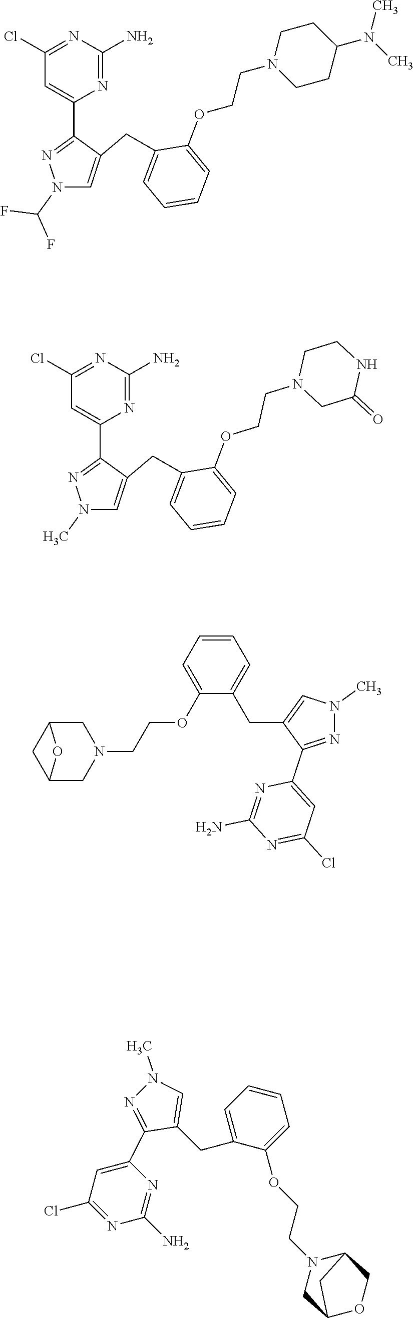 [SMILES: CN(C)C1CCN(CCOc2ccccc2Cc2cn(C(F)F)nc2-c2cc(Cl)nc(N)n2)CC1 ; Cn1cc(Cc2ccccc2OCCN2CCNC(=O)C2)c(-c2cc(Cl)nc(N)n2)n1 ; Cn1cc(Cc2ccccc2OCCN2CC3CC(C2)O3)c(-c2cc(Cl)nc(N)n2)n1 ; Cn1cc(Cc2ccccc2OCCN2C[C@H]3C[C@@H]2CO3)c(-c2cc(Cl)nc(N)n2)n1] 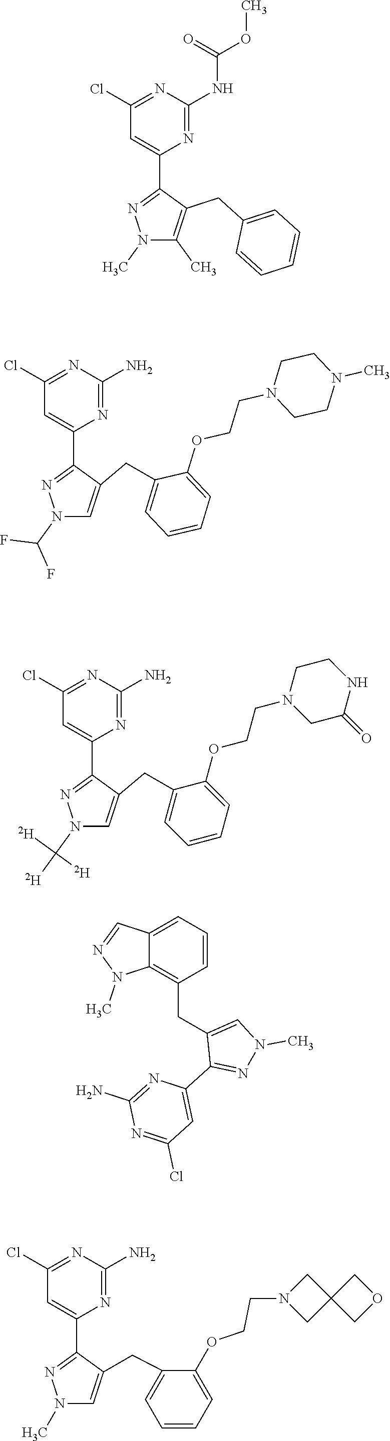 [SMILES: COC(=O)Nc1nc(Cl)cc(-c2nn(C)c(C)c2Cc2ccccc2)n1 ; CN1CCN(CCOc2ccccc2Cc2cn(C(F)F)nc2-c2cc(Cl)nc(N)n2)CC1 ; [2H]C([2H])([2H])n1cc(Cc2ccccc2OCCN2CCNC(=O)C2)c(-c2cc(Cl)nc(N)n2)n1 ; Cn1cc(Cc2cccc3cnn(C)c23)c(-c2cc(Cl)nc(N)n2)n1 ; Cn1cc(Cc2ccccc2OCCN2CC3(COC3)C2)c(-c2cc(Cl)nc(N)n2)n1] 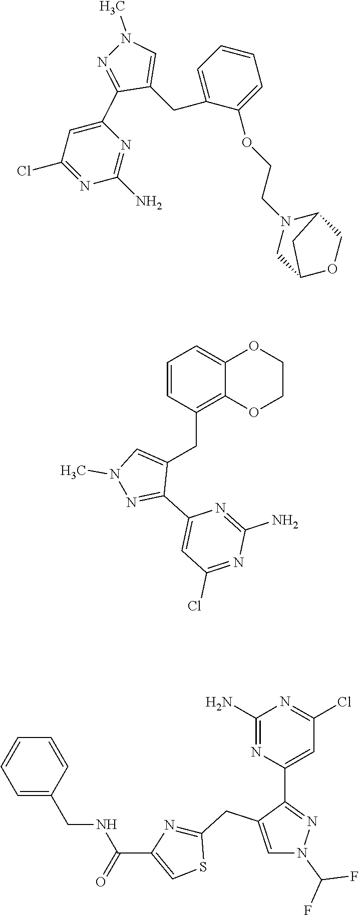 [SMILES: Cn1cc(Cc2ccccc2OCCN2C[C@@H]3C[C@H]2CO3)c(-c2cc(Cl)nc(N)n2)n1 ; Cn1cc(Cc2cccc3c2OCCO3)c(-c2cc(Cl)nc(N)n2)n1 ; Nc1nc(Cl)cc(-c2nn(C(F)F)cc2Cc2nc(C(=O)NCc3ccccc3)cs2)n1] 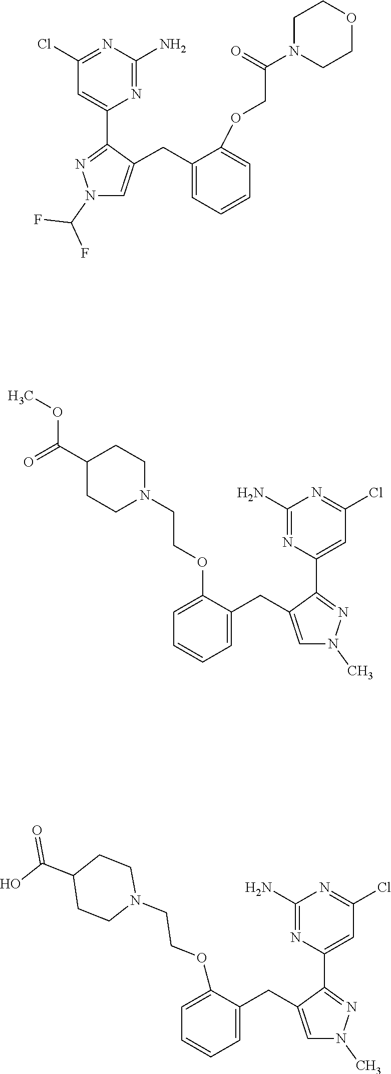 [SMILES: Nc1nc(Cl)cc(-c2nn(C(F)F)cc2Cc2ccccc2OCC(=O)N2CCOCC2)n1 ; COC(=O)C1CCN(CCOc2ccccc2Cc2cn(C)nc2-c2cc(Cl)nc(N)n2)CC1 ; Cn1cc(Cc2ccccc2OCCN2CCC(C(=O)O)CC2)c(-c2cc(Cl)nc(N)n2)n1] 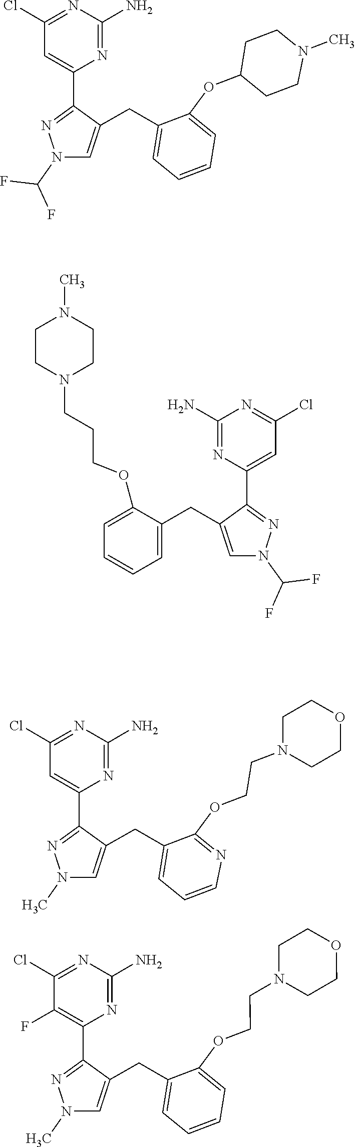 [SMILES: CN1CCC(Oc2ccccc2Cc2cn(C(F)F)nc2-c2cc(Cl)nc(N)n2)CC1 ; CN1CCN(CCCOc2ccccc2Cc2cn(C(F)F)nc2-c2cc(Cl)nc(N)n2)CC1 ; Cn1cc(Cc2cccnc2OCCN2CCOCC2)c(-c2cc(Cl)nc(N)n2)n1 ; Cn1cc(Cc2ccccc2OCCN2CCOCC2)c(-c2nc(N)nc(Cl)c2F)n1] 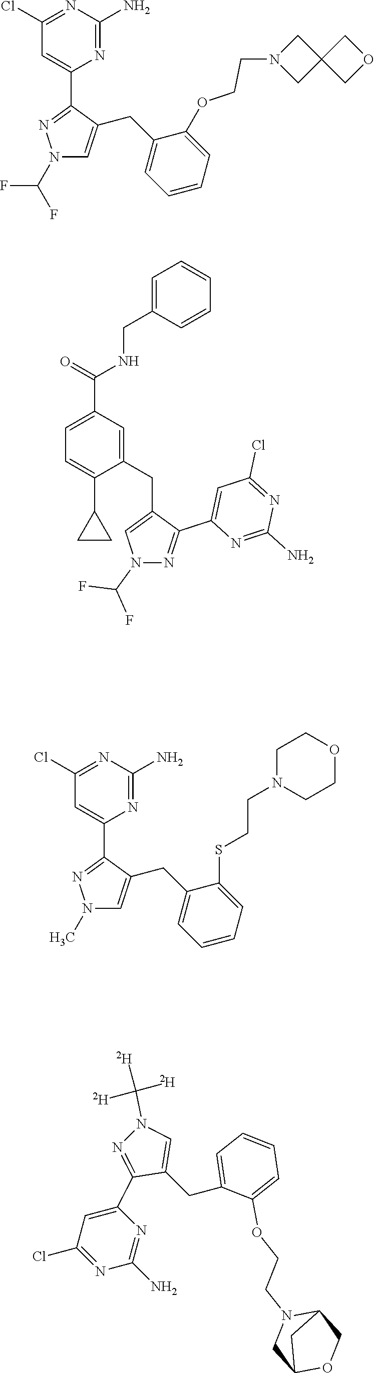 [SMILES: Nc1nc(Cl)cc(-c2nn(C(F)F)cc2Cc2ccccc2OCCN2CC3(COC3)C2)n1 ; Nc1nc(Cl)cc(-c2nn(C(F)F)cc2Cc2cc(C(=O)NCc3ccccc3)ccc2C2CC2)n1 ; Cn1cc(Cc2ccccc2SCCN2CCOCC2)c(-c2cc(Cl)nc(N)n2)n1 ; [2H]C([2H])([2H])n1cc(Cc2ccccc2OCCN2C[C@H]3C[C@@H]2CO3)c(-c2cc(Cl)nc(N)n2)n1] 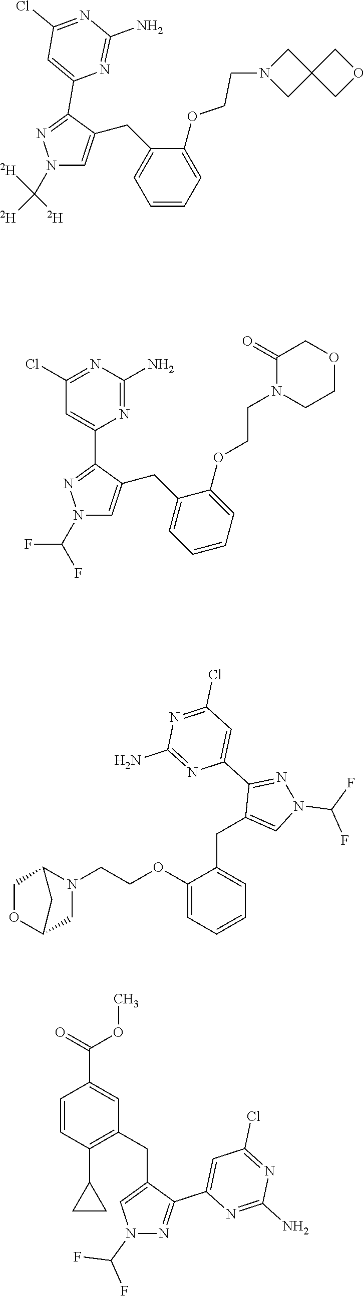 [SMILES: [2H]C([2H])([2H])n1cc(Cc2ccccc2OCCN2CC3(COC3)C2)c(-c2cc(Cl)nc(N)n2)n1 ; Nc1nc(Cl)cc(-c2nn(C(F)F)cc2Cc2ccccc2OCCN2CCOCC2=O)n1 ; Nc1nc(Cl)cc(-c2nn(C(F)F)cc2Cc2ccccc2OCCN2C[C@H]3C[C@@H]2CO3)n1 ; COC(=O)c1ccc(C2CC2)c(Cc2cn(C(F)F)nc2-c2cc(Cl)nc(N)n2)c1] 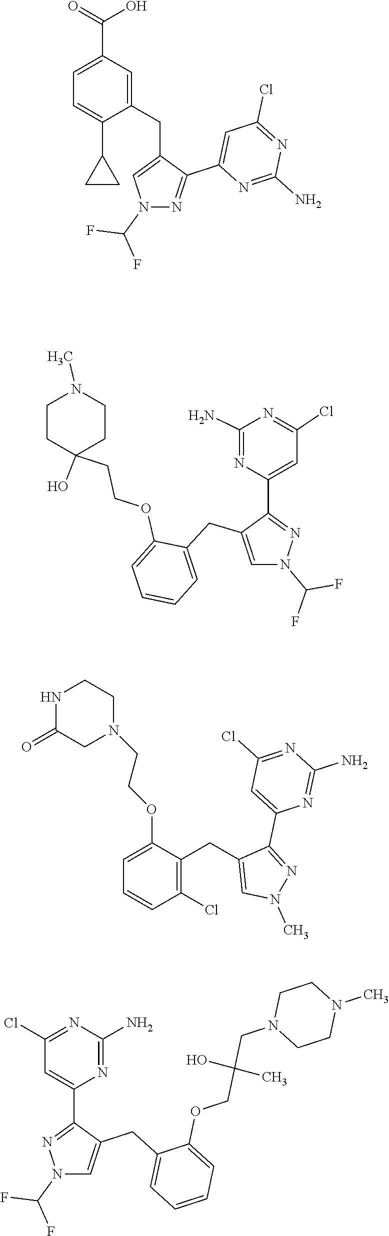 [SMILES: Nc1nc(Cl)cc(-c2nn(C(F)F)cc2Cc2cc(C(=O)O)ccc2C2CC2)n1 ; CN1CCC(O)(CCOc2ccccc2Cc2cn(C(F)F)nc2-c2cc(Cl)nc(N)n2)CC1 ; Cn1cc(Cc2c(Cl)cccc2OCCN2CCNC(=O)C2)c(-c2cc(Cl)nc(N)n2)n1 ; CN1CCN(CC(C)(O)COc2ccccc2Cc2cn(C(F)F)nc2-c2cc(Cl)nc(N)n2)CC1] 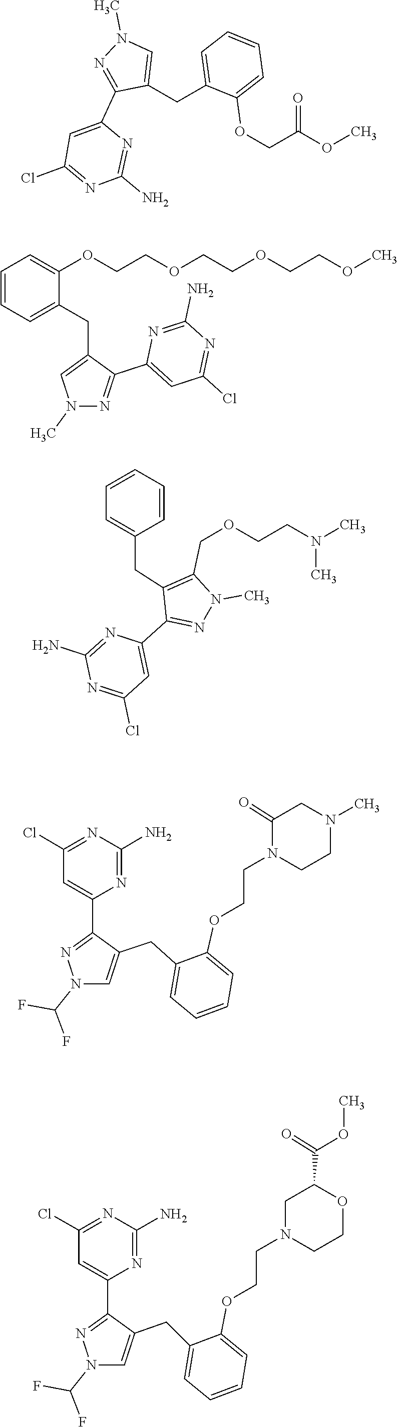 [SMILES: COC(=O)COc1ccccc1Cc1cn(C)nc1-c1cc(Cl)nc(N)n1 ; COCCOCCOCCOc1ccccc1Cc1cn(C)nc1-c1cc(Cl)nc(N)n1 ; CN(C)CCOCc1c(Cc2ccccc2)c(-c2cc(Cl)nc(N)n2)nn1C ; CN1CCN(CCOc2ccccc2Cc2cn(C(F)F)nc2-c2cc(Cl)nc(N)n2)C(=O)C1 ; COC(=O)[C@H]1CN(CCOc2ccccc2Cc2cn(C(F)F)nc2-c2cc(Cl)nc(N)n2)CCO1] 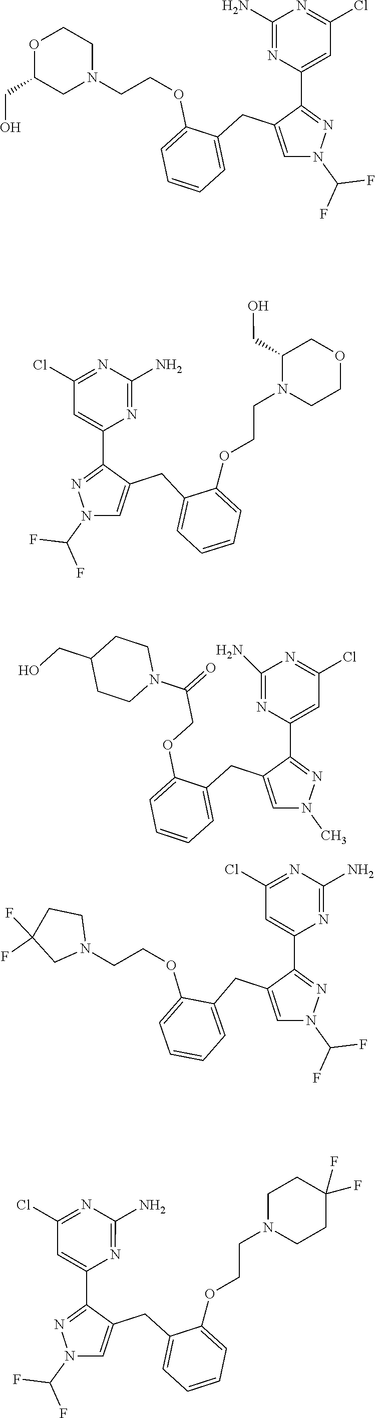 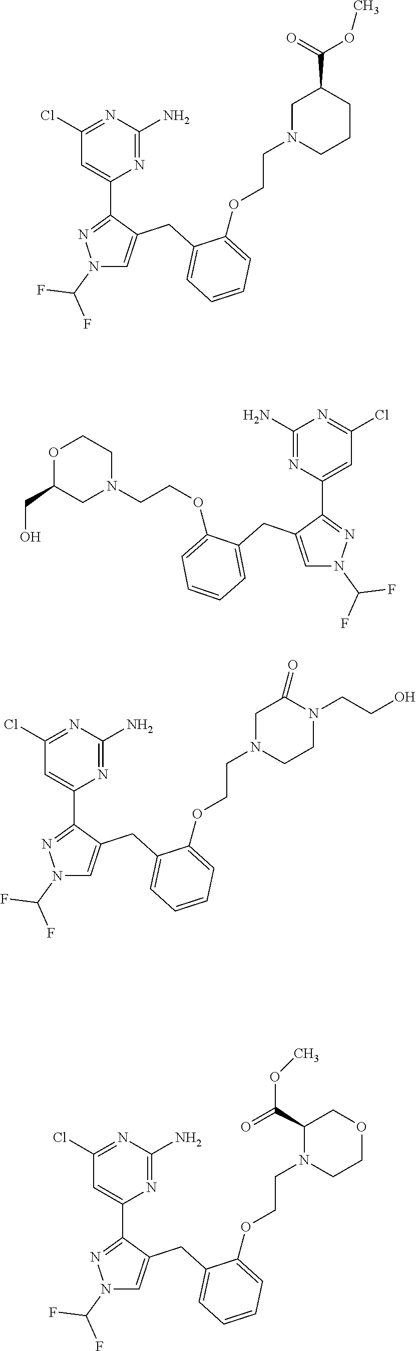 [SMILES: COC(=O)[C@H]1CCCN(CCOc2ccccc2Cc2cn(C(F)F)nc2-c2cc(Cl)nc(N)n2)C1 ; Nc1nc(Cl)cc(-c2nn(C(F)F)cc2Cc2ccccc2OCCN2CCO[C@H](CO)C2)n1 ; Nc1nc(Cl)cc(-c2nn(C(F)F)cc2Cc2ccccc2OCCN2CCN(CCO)C(=O)C2)n1 ; COC(=O)[C@H]1COCCN1CCOc1ccccc1Cc1cn(C(F)F)nc1-c1cc(Cl)nc(N)n1] 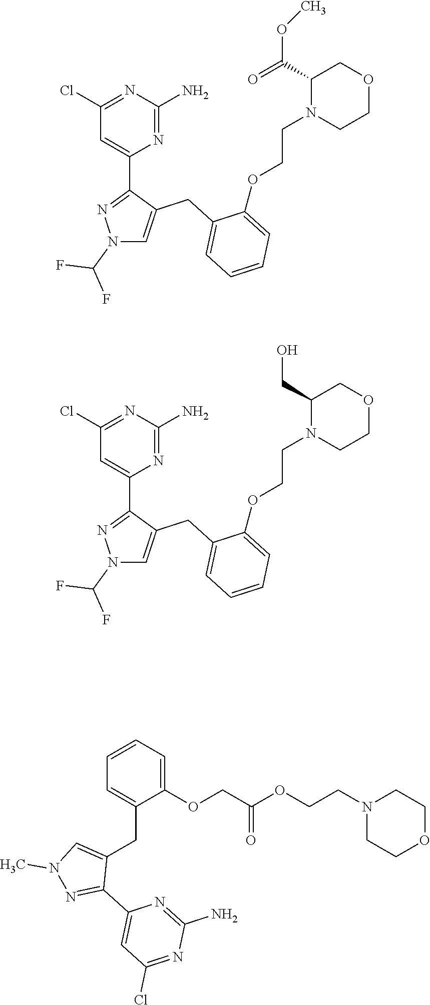 [SMILES: COC(=O)[C@@H]1COCCN1CCOc1ccccc1Cc1cn(C(F)F)nc1-c1cc(Cl)nc(N)n1 ; Nc1nc(Cl)cc(-c2nn(C(F)F)cc2Cc2ccccc2OCCN2CCOC[C@@H]2CO)n1 ; Cn1cc(Cc2ccccc2OCC(=O)OCCN2CCOCC2)c(-c2cc(Cl)nc(N)n2)n1] 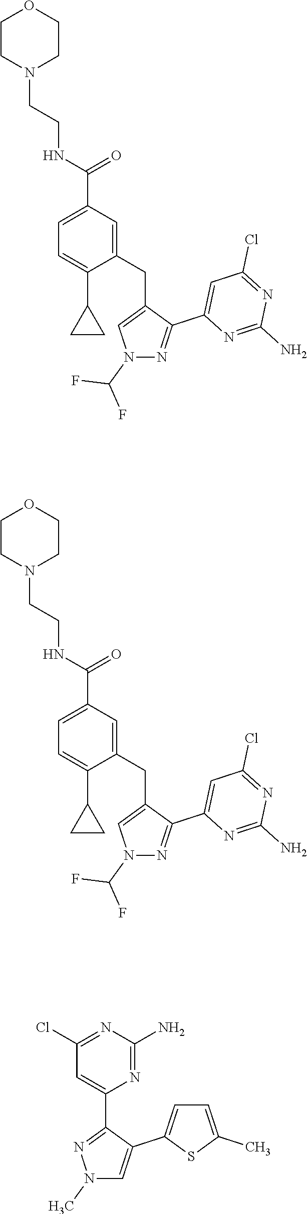 [SMILES: Nc1nc(Cl)cc(-c2nn(C(F)F)cc2Cc2cc(C(=O)NCCN3CCOCC3)ccc2C2CC2)n1 ; Nc1nc(Cl)cc(-c2nn(C(F)F)cc2Cc2cc(C(=O)NCCN3CCOCC3)ccc2C2CC2)n1 ; Cc1ccc(-c2cn(C)nc2-c2cc(Cl)nc(N)n2)s1] 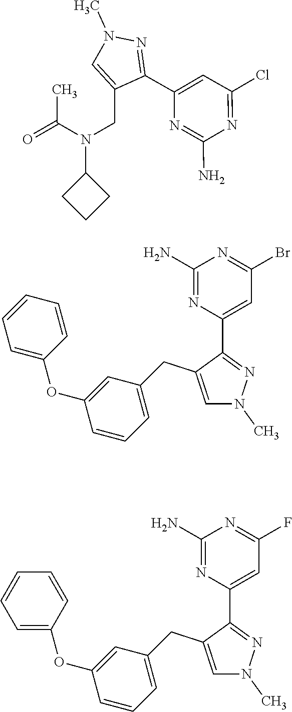 [SMILES: CC(=O)N(Cc1cn(C)nc1-c1cc(Cl)nc(N)n1)C1CCC1 ; Cn1cc(Cc2cccc(Oc3ccccc3)c2)c(-c2cc(Br)nc(N)n2)n1 ; Cn1cc(Cc2cccc(Oc3ccccc3)c2)c(-c2cc(F)nc(N)n2)n1] 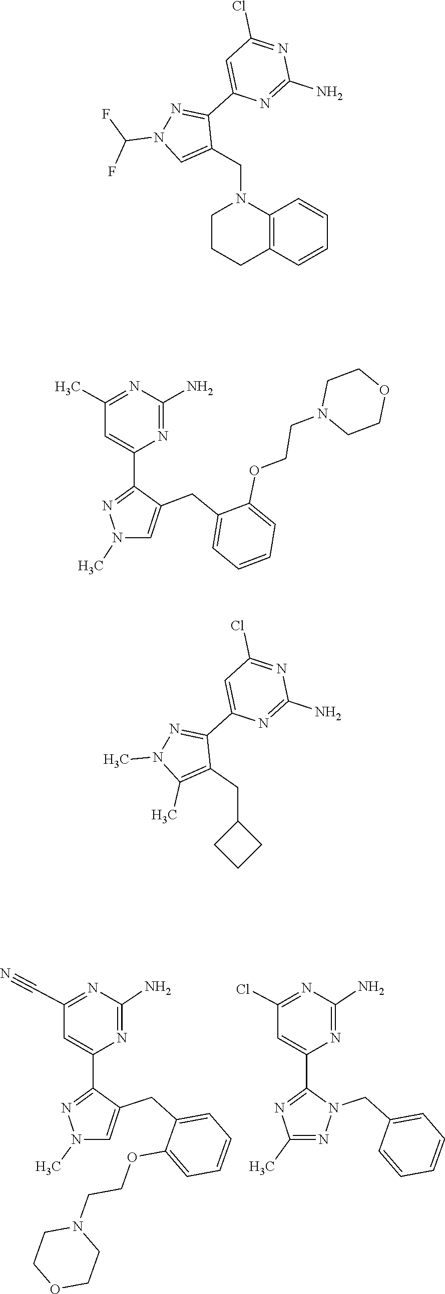 [SMILES: Nc1nc(Cl)cc(-c2nn(C(F)F)cc2CN2CCCc3ccccc32)n1 ; Cc1cc(-c2nn(C)cc2Cc2ccccc2OCCN2CCOCC2)nc(N)n1 ; Cc1c(CC2CCC2)c(-c2cc(Cl)nc(N)n2)nn1C ; Cn1cc(Cc2ccccc2OCCN2CCOCC2)c(-c2cc(C#N)nc(N)n2)n1 ; Cc1nc(-c2cc(Cl)nc(N)n2)n(Cc2ccccc2)n1] 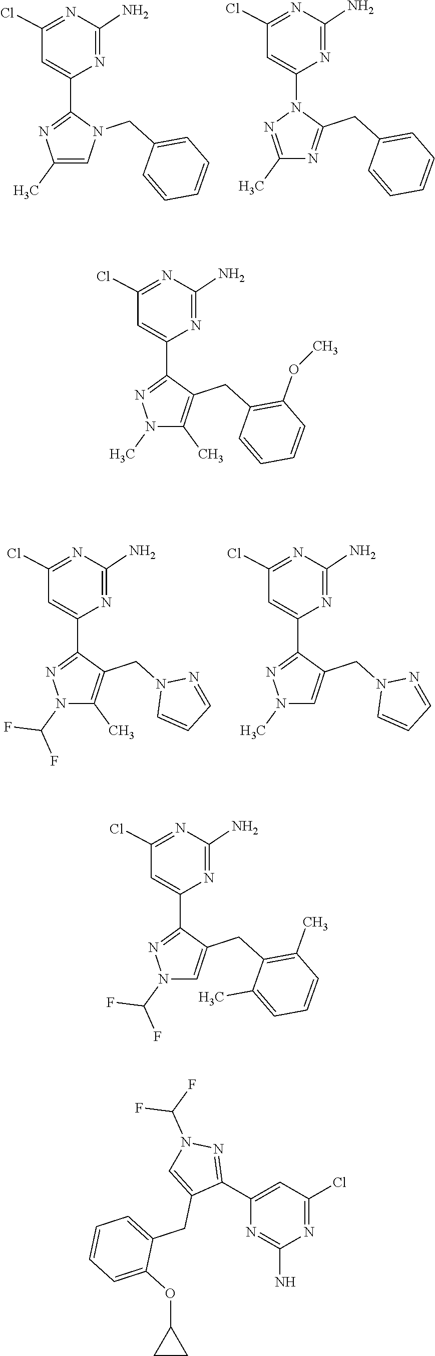 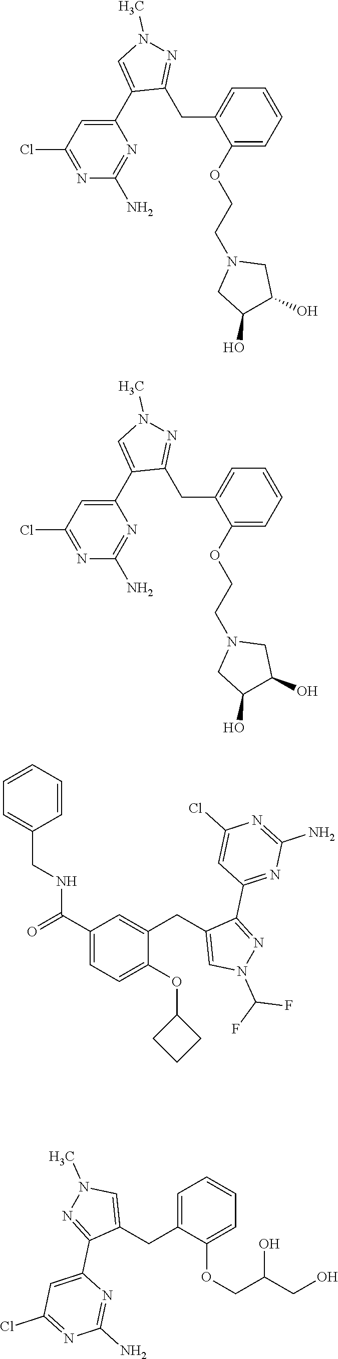 [SMILES: Cn1cc(-c2cc(Cl)nc(N)n2)c(Cc2ccccc2OCCN2C[C@H](O)[C@@H](O)C2)n1 ; Cn1cc(-c2cc(Cl)nc(N)n2)c(Cc2ccccc2OCCN2C[C@@H](O)[C@@H](O)C2)n1 ; Nc1nc(Cl)cc(-c2nn(C(F)F)cc2Cc2cc(C(=O)NCc3ccccc3)ccc2OC2CCC2)n1 ; Cn1cc(Cc2ccccc2OCC(O)CO)c(-c2cc(Cl)nc(N)n2)n1] 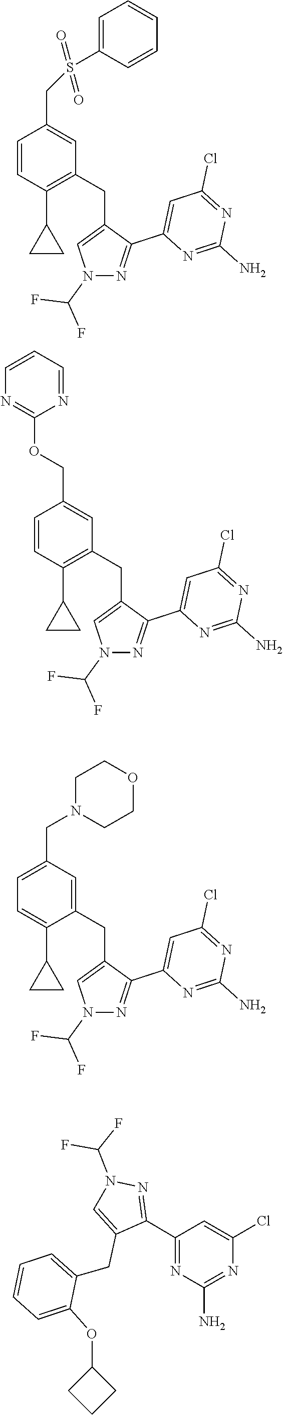 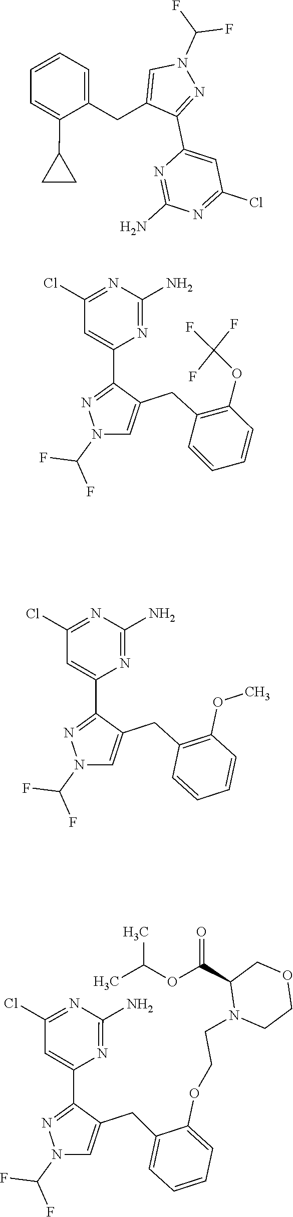 [SMILES: Nc1nc(Cl)cc(-c2nn(C(F)F)cc2Cc2ccccc2C2CC2)n1 ; Nc1nc(Cl)cc(-c2nn(C(F)F)cc2Cc2ccccc2OC(F)(F)F)n1 ; COc1ccccc1Cc1cn(C(F)F)nc1-c1cc(Cl)nc(N)n1 ; CC(C)OC(=O)[C@H]1COCCN1CCOc1ccccc1Cc1cn(C(F)F)nc1-c1cc(Cl)nc(N)n1] 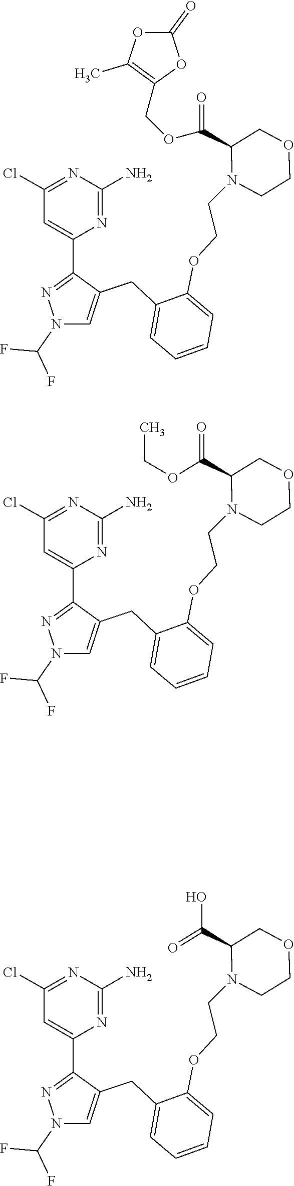 [SMILES: Cc1oc(=O)oc1COC(=O)[C@H]1COCCN1CCOc1ccccc1Cc1cn(C(F)F)nc1-c1cc(Cl)nc(N)n1 ; CCOC(=O)[C@H]1COCCN1CCOc1ccccc1Cc1cn(C(F)F)nc1-c1cc(Cl)nc(N)n1 ; Nc1nc(Cl)cc(-c2nn(C(F)F)cc2Cc2ccccc2OCCN2CCOC[C@@H]2C(=O)O)n1] 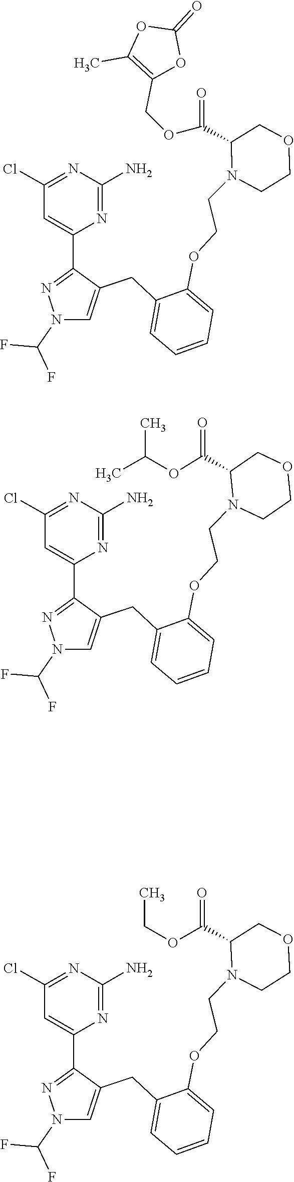 [SMILES: Cc1oc(=O)oc1COC(=O)[C@@H]1COCCN1CCOc1ccccc1Cc1cn(C(F)F)nc1-c1cc(Cl)nc(N)n1 ; CC(C)OC(=O)[C@@H]1COCCN1CCOc1ccccc1Cc1cn(C(F)F)nc1-c1cc(Cl)nc(N)n1 ; CCOC(=O)[C@@H]1COCCN1CCOc1ccccc1Cc1cn(C(F)F)nc1-c1cc(Cl)nc(N)n1] 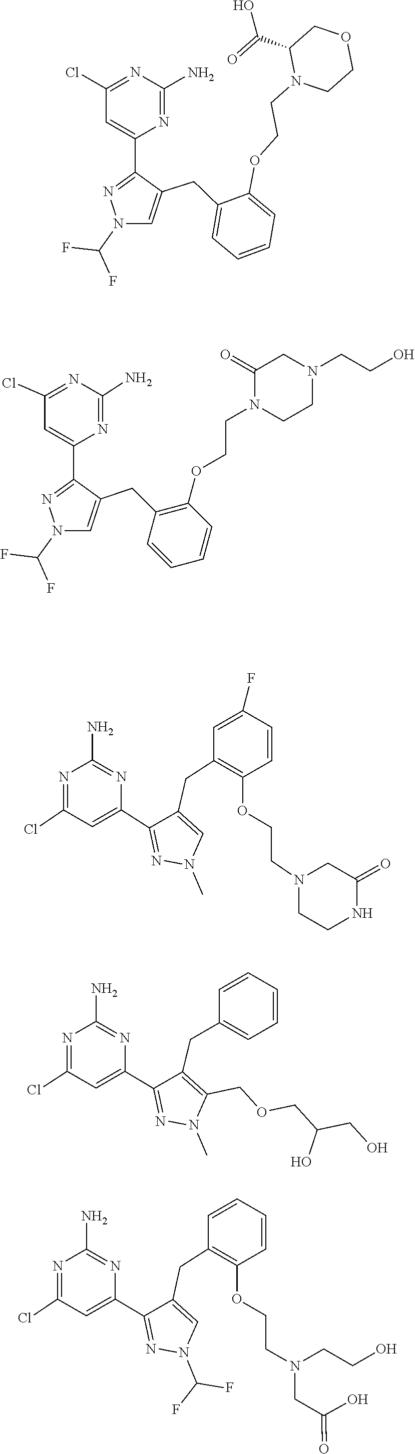 [SMILES: Nc1nc(Cl)cc(-c2nn(C(F)F)cc2Cc2ccccc2OCCN2CCOC[C@H]2C(=O)O)n1 ; Nc1nc(Cl)cc(-c2nn(C(F)F)cc2Cc2ccccc2OCCN2CCN(CCO)CC2=O)n1 ; Cn1cc(Cc2cc(F)ccc2OCCN2CCNC(=O)C2)c(-c2cc(Cl)nc(N)n2)n1 ; Cn1nc(-c2cc(Cl)nc(N)n2)c(Cc2ccccc2)c1COCC(O)CO ; Nc1nc(Cl)cc(-c2nn(C(F)F)cc2Cc2ccccc2OCCN(CCO)CC(=O)O)n1] 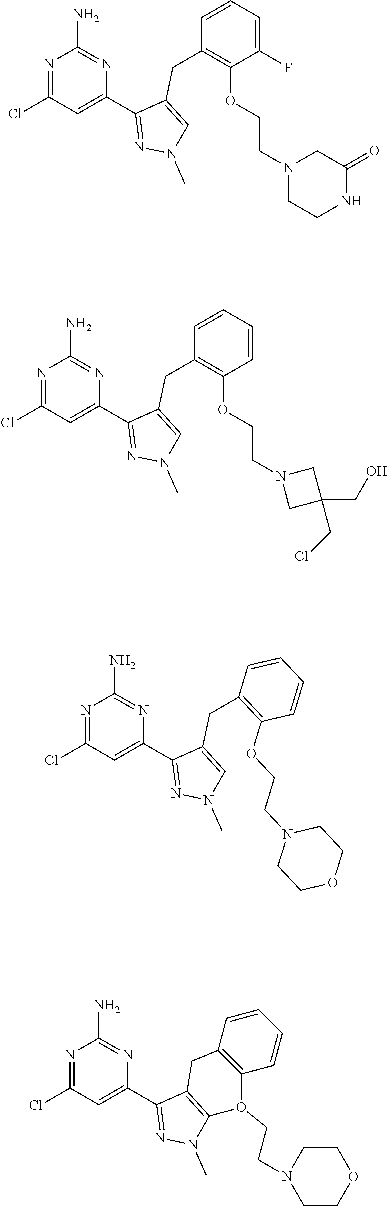 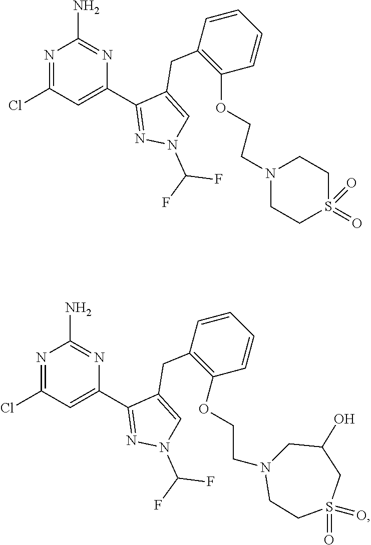 [SMILES: Nc1nc(Cl)cc(-c2nn(C(F)F)cc2Cc2ccccc2OCCN2CCS(=O)(=O)CC2)n1 ; Nc1nc(Cl)cc(-c2nn(C(F)F)cc2Cc2ccccc2OCCN2CCS(=O)(=O)CC(O)C2)n1]
and pharmaceutically acceptable salts thereof.

50. The compound of any one of the preceding claims, or a pharmaceutically acceptable salt thereof, wherein the compound has an off-rate (T_{1/2}) of greater than 20 seconds from a soluble adenylyl cyclase (sAC) protein.

51. The compound of any one of the preceding claims, or a pharmaceutically acceptable salt thereof, wherein the compound has an off-rate (T_{1/2}) of greater than 1,000 seconds from a sAC protein.

52. The compound of any one of the preceding claims, or a pharmaceutically acceptable salt thereof, wherein the compound has an off-rate (T_{1/2}) of greater than 10,000 seconds from a sAC protein.

53. The compound of any one of the preceding claims, or a pharmaceutically acceptable salt thereof, wherein the compound has an off-rate (T_{1/2}) of from 25-20,000 seconds from a sAC protein.

54. The compound of any one of the preceding claims, or a pharmaceutically acceptable salt thereof, wherein the compound has an off-rate (T_{1/2}) of from 1,000-20,000 seconds from a sAC protein.

55. A pharmaceutical composition comprising a compound of any one of claims 1-54, or a pharmaceutically acceptable salt thereof, and a pharmaceutically acceptable carrier or excipient.

56. A method for contraception, the method comprising administering to a subject a compound of any one of claims 1-54, or a pharmaceutically acceptable salt thereof, or a pharmaceutical composition of claim 55.

57. The method of claim 56, wherein the method is a method for male contraception; and the subject is a male subject.

58. The method of claim 57, wherein the compound, or pharmaceutically acceptable salt thereof, or pharmaceutical composition thereof, is administered orally to the male subject.

59. The method of claim 56, wherein the method is a method for female contraception; and the subject is a female subject.

60. The method of claim 59, wherein the compound, or pharmaceutically acceptable salt thereof, or pharmaceutical composition thereof, is administered intravaginally to the female subject.

61. The method of claim 59, wherein the compound, or pharmaceutically acceptable salt thereof, or pharmaceutical composition thereof, is administered orally to the female subject.

62. A method for treating an ocular condition in a subject, the method comprising administering to the subject a compound of any one of claims 1-54, or a pharmaceutically acceptable salt thereof, or a pharmaceutical composition of claim 55.

63. The method of claim 62, wherein the ocular condition is ocular hypotony.

64. A method for increasing intraocular pressure (IOP) in a subject, the method comprising administering to the subject a compound of any one of claims 1-54, or a pharmaceutically acceptable salt thereof, or a pharmaceutical composition of claim 55.

65. A method for treating and/or preventing a liver disease in a subject, the method comprising administering to the subject a compound of any one of claims 1-54, or a pharmaceutically acceptable salt thereof, or a pharmaceutical composition of claim 55.

66. The method of claim 65, wherein the liver disease is non-alcoholic steatohepatitis (NASH).

67. The method of claim 65, wherein the method is a method of preventing the development of NASH in a subject.

68. The method of claim 65, wherein the method is a method of preventing the worsening or progression of NASH in a subject.

69. A method for treating psoriasis in a subject, the method comprising administering to the subject a compound of any one of claims 1-54, or a pharmaceutically acceptable salt thereof, or a pharmaceutical composition of claim 55.

70. A method for treating an inflammatory or autoimmune disease in a subject, the method comprising administering to the subject a compound of any one of claims 1-54, or a pharmaceutically acceptable salt thereof, or a pharmaceutical composition of claim 55.

71. The method of claim 70, wherein the inflammatory or autoimmune disease is a Th17-mediated inflammatory or autoimmune disease.

72. The method of claim 70, wherein the inflammatory or autoimmune disease is a type 17 inflammatory or autoimmune disease.

73. A method for treating a disease in a subject, the method comprising administering to the subject a compound of any one of claims 1-54, or a pharmaceutically acceptable salt thereof, or a pharmaceutical composition of claim 55.

74. The method of claim 73, wherein the disease is typically associated with the activity of a sAC enzyme.

75. A method for inhibiting the activity of soluble adenylyl cyclase (sAC) in a subject or biological sample, the method comprising administering to the subject or contacting the biological sample with a compound of any one of claims 1-54, or a pharmaceutically acceptable salt thereof, or a pharmaceutical composition of claim 55.

76. The method of any one of claims 1-75, wherein the subject is a human.

77. The method of any one of claims 1-75, wherein the subject is a non-human mammal.

78. The method of any one of claims 1-75, wherein the subject is a canine.

79. The method of claim 75, wherein the inhibiting occurs in vivo in a subject.

80. The method of claim 75, wherein the inhibiting occurs in vitro.

81. A compound of any one of claims 1-54, or a pharmaceutically acceptable salt thereof, or a pharmaceutical composition of claim 55, for use in treating a disease in a subject.

82. Use of a compound of any one of claims 1-54, or a pharmaceutically acceptable salt thereof, or a pharmaceutical composition of claim 555, for the manufacture of a medicament for treating a disease in a subject.

83. A method for male contraception comprising administering to a male subject a soluble adenylyl cyclase (sAC) inhibitor with an off-rate (T_{1/2}) of greater than 20 seconds from a sAC protein.

84. The method of claim 83, wherein the sAC inhibitor has an off-rate (T_{1/2}) of greater than 1,000 seconds from a sAC protein.

85. The method of claim 83, wherein the sAC inhibitor has an off-rate (T_{1/2}) of greater than 10,000 seconds from a sAC protein.

86. The method of claim 83, wherein the sAC inhibitor has an off-rate (T_{1/2}) of from 25-20,000 seconds from a sAC protein.

87. The method of claim 83, wherein the sAC inhibitor has an off-rate (T_{1/2}) of from 1,000-20,000 seconds from a sAC protein.

88. A kit comprising:
(i) an oral contraceptive pill for administration to a male comprising a compound of any one of the preceding claims, or a pharmaceutically acceptable salt thereof, or a pharmaceutical composition thereof; and
(ii) an oral contraceptive pill for administration to a female comprising a compound of any one of the preceding claims, or a pharmaceutically acceptable salt thereof, or a pharmaceutical composition thereof; and optionally instructions for use.
